(19) United States
(12) Patent Application Publication
(10) Pub. No.: US 2010/0121052 A1
(43) Pub. Date: May 13, 2010

(54) Novel compounds for treating proliferative diseases

(76) Inventors: Rama Jain, Fremont, CA (US); Xiaodong Lin, Walnut Creek, CA (US); Simon C. Ng, Walnut Creek, CA (US); Keith B. Pfister, San Ramon, CA (US); Savithri Ramurthy, Walnut Creek, CA (US); Alice Rico, Castro Valley, CA (US); Sharadha Subramanian, San Ramon, CA (US); Xiaojing Michael Wang, Livermore, CA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/456,032

(22) Filed: Jun. 9, 2009

Related U.S. Application Data
(60) Provisional application No. 61/074,556, filed on Jun. 20, 2008.

(51) Int. Cl. C07D 239/84; C07D 417/10; C07D 401/12; C07D 413/12; C07D 401/14; C07D 413/14

(52) U.S. Cl. 544/119; 544/292; 544/284

(57) ABSTRACT

The invention provides novel compounds that are inhibitors of PDK1. Also provided are pharmaceutical compositions including the compounds, and methods of treating proliferative diseases, such as cancers, with the compounds or compositions.

This invention claims priority to U.S. Ser. No. 61/074,556 filed Jun. 20, 2009, the contents of which are incorporated herein in their entirety.

### TECHNICAL FIELD

The invention relates to small molecule inhibitors of 3-phosphoinositide-dependent kinase (PDK1/PDPK1), and their use therapeutics in the treatment of cellular proliferative diseases.

### BACKGROUND

PDK1 (3-Phosphoinositide-dependent kinase 1) is a serine/threonine kinase belonging to the AGC kinase super family. PDK1 was first identified as the upstream kinase responsible for activating protein kinase B/AKT in the presence of phosphoinositide lipids (PIP₃). PDK1 activates AKT by phosphorylating a specific residue (threonine 308) located in the activation loop of this kinase. Subsequent research has shown that PDK1 is responsible for phosphorylating the activation-loop of many AGC kinases including p90 ribosomal S6 kinase (RSK), protein kinase C family members (PKC), p70 ribosomal S6 kinase (70S6K), and the serum and glucocorticoid-induced protein kinase (SGK). Thus, PDK1 is a central activator of multiple signaling pathways that are involved in cell proliferation, survival and control of apoptosis. Importantly, alterations in these signaling pathways are frequently observed in a variety of human cancers. For example, AKT is highly activated in a large percentage of common tumor types including melanoma, breast, lung, prostate and ovarian cancers. RSK levels are elevated in prostate cancers, and an RSK-specific inhibitor (SL0101) has recently been shown to inhibit the proliferation of multiple prostate cancer cell lines. Similarly, PKCε has been shown to play an important role in regulating apoptosis and promoting survival of glioma cells.

The human PDK1 gene encodes a 556 amino acid protein with an amino-terminal catalytic domain and a non-catalytic carboxy terminal containing a pleckstrin homology domain (PH). Recent studies suggest that PDK1 is a constitutively active kinase, and that PDK1 regulation occurs through the localization or conformational state of PDK1 target proteins. For example, the PH domain of PDK1 is required for the binding of PIP₃ lipids produced by PI3kinase (PI3K). PDK1 binding of PIP₃ lipids results in membrane co-localization with AKT, another PH domain containing protein. Once co-localized, PDK1 activates AKT by phosphorylating threonine308. Alternatively, PDK1 can activate other AGC kinases independent of PIP₃ lipids by binding directly to a conserved motif found on these targets. Because PDK1 regulates two distinct classes of downstream signaling substrates (PI3K-dependent and independent targets), inhibitors of this enzyme could have important therapeutic value in a variety of human cancers. For instance, PDK1 inhibitors could be efficacious in tumors in which the PI3K signaling pathway is upregulated due to activating mutations, amplification of PI3K itself or its upstream receptor tyrosine kinases, or deletion of PTEN, the phosphatase the counteracts PI3K activity. The finding that mice expressing half the normal amount of PTEN are protected from developing a wide range of tumors by reducing PDK1 expression levels supports this idea. Alternatively, PDK1 inhibitors could be useful in treating cancers driven by PIP₃-independent PDK1 signaling pathways (e.g. K-ras or H-ras driven cancers). Finally, the recent identification of PDK1 mutations (PDK1^{T354M}, PDK1^{D527E}) in human colorectal cancers suggests that inhibitors of this kinase may have therapeutic value by directly inhibiting either wild-type or mutant forms of this protein. See, Parsons et al., Nature 436, 792 (11 Aug. 2005) “Colorectal cancer: Mutations in a signaling pathway.”

In summary, PDK1 is a central activator of several signaling pathways that are frequently altered in human cancers making it an attractive target for therapeutic intervention.

### SUMMARY

In one aspect, the present invention provides compounds of Formula (Ia):

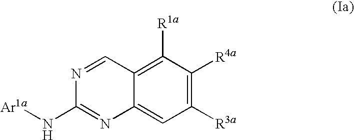

or pharmaceutically acceptable salts thereof, wherein:

R^{1a} is selected from H and halogen;

R^{3a} is selected from C₂₋₆ heterocycloalkyloxy and C₁₋₆ heteroaryl-C₁₋₄-alkoxy; wherein said C₂₋₆ heterocycloalkyloxy and C₁₋₆ heteroaryl-C₁₋₄-alkoxy are each optionally substituted by a R^{w} group;

R^{4a} is selected from H, a thiazole ring, a pyrazole ring, a triazole ring, a tetrazole ring, a pyridine ring, C₃₋₆ cycloalkyl, cyano, halogen, C₂₋₆ alkynyl, C₁₋₆ heteroaryl-C₁₋₄-alkyl, C₁₋₆ heteroaryl-C₁₋₄-alkynyl, —C(═O)R^{a}, and —C(═O)NR^{b}R^{c}; wherein said thiazole ring, pyrazole ring, triazole ring, tetrazole ring, pyridine ring, C₁₋₆ heteroaryl-C₁₋₄-alkyl and C₁₋₆ heteroaryl-C₁₋₄-alkynyl are each optionally substituted by 1 or 2 independently selected R^{x″} groups;

Ar^{1a} is selected from phenyl optionally substituted at the meta position by one R^{y′} or, alternatively, at the para position by one R^{y″} group;

each R^{w} is independently selected from halogen, C₁₋₆ alkyl, amino, C₁₋₆ alkylamino, di-C₁₋₄-alkylamino, C₁₋₆ alkoxycarbonyl, and C₁₋₆ alkylcarbamyl; wherein said C₁₋₆ alkyl and C₁₋₆ alkylamino are each optionally substituted by a group selected from hydroxyl, C₁₋₆ alkoxy, amino, C₁₋₆alkylamino, and di-C₁₋₄-alkylamino;

each R^{x″} is independently selected from halogen, hydroxyl, C₁₋₆ alkyl, C₁₋₆ alkoxycarbonyl, and carbamyl; wherein said C₁₋₆ alkyl and C₁₋₆ alkoxycarbonyl are each optionally substituted by a group selected from hydroxyl, amino, C₁₋₄ alkylamino, and aminosulfonyl;

R^{y′} is selected from halogen, C₁₋₆ alkoxy, C₁₋₆ alkyl, carbamyl, aminosulfonyl, and C₁₋₆ alkylsulfonylamino; wherein said C₁₋₆ alkyl and C₁₋₆ alkoxy are each substituted by 1 or 2 groups independently selected from hydroxyl, amino, C₁₋₄ alkylamino, and aminosulfonyl;

R^{y″} is selected from C₁₋₆ haloalkyl;

R^{a} is selected from H, C₁₋₆ alkoxy and C₂₋₆ heterocycloalkyl;

R^{b} is selected from H and C₁₋₆ alkyl; and

R^{c} is selected from C₁₋₆ alkyl, C₃₋₆ cycloalkyl, C₁₋₆ heteroaryl, C₂₋₆ heterocycloalkyl, C₂₋₆ heterocycloalkyl-C₁₋₄-alkyl, and C₁₋₆ heteroaryl-C₁₋₄-alkyl; wherein said C₁₋₆ alkyl is optionally substituted by a group selected from hydroxyl and C₁₋₄ alkoxy.

In some embodiments, the following provisos apply:
(a) when R^{3a} is piperidin-4-yloxy or N-methylpiperidin-4-yloxy, then R⁴ is not selected from thiazol-2-yl, thiazol-4-yl, thiazol-5-yl, 5-methylthiazol-2-yl, 5-(hydroxymethyl)thiazol-2-yl, 5-(hydroxymethyl)thiazol-4-yl, pyridin-2-yl, pyridin-4-yl, pyridin-3-yl, 4-methylpyridin-3-yl, 5-chloropyridin-4-yl, 3-methylpyridin-2-yl, and 1-methylpyrazol-5-yl;
(b) when R^{4a} is ethynyl and Ar^{1a} is 3-fluorophenyl, then R³ is not selected from pyridin-3-ylmethoxy, 6-chloropyridin-3-ylmethoxy, pyridin-2-ylmethoxy, pyridin-4-ylmethoxy, 1-(pyridin-4-yl)ethoxy, 6-methoxypyridin-2-ylmethoxy, thiazol-5-ylmethoxy, pyrazin-2-ylmethoxy, 5-methylisooxazol-3-yl, azetidin-3-yloxy, N-methylazetidin-3-yloxy, N-isopropylazetidin-3-yloxy, pyrrolidin-3-yloxy, N-methyl, and pyrrolidin-3-yloxy;
(c) when R^{4a} is ethynyl, then Ar^{1a} is not selected from 3-(2-hydroxypropan-2-yl)phenyl, 3-(1-hydroxyethyl)phenyl, 3-aminosulfonylphenyl, phenyl, 3-(methylsulfonylamino)phenyl, 3-(N,N-dimethylaminomethyl)phenyl, 3-aminosulfonylphenyl, and 3-carbamylphenyl;
(d) when R^{4a} is thiazol-2-yl, then Ar^{1a} is not selected from 3-(methylaminosulfonyl)phenyl, 3-chlorophenyl, 3-carbamylphenyl, 3-fluorophenyl, 2-(hydroxypropan-2-yl)phenyl, 1-hydroxyethyl, 3-aminosulfonylphenyl, and phenyl;
(e) when R^{4a} is thiazol-2-yl and Ar^{1a} is 3-fluorophenyl, then R³ is not selected from pyridin-3-ylmethoxy, 6-chloropyridin-3-ylmethoxy, 2-chloropyridin-4-ylmethoxy, 5-methylisooxazol-3-yl, azetidin-3-yloxy, N-methylazetidin-3-yloxy, N-isopropylazetidin-3-yloxy, pyrrolidin-3-yloxy, N-methyl, pyrrolidin-3-yloxy, piperidin-4-yloxy, and N-methylpiperidin-4-yloxy;
(f) when R^{4a} is bromo, then Ar^{1a} is not selected from phenyl, 3-fluorophenyl, 2-(hydroxypropan-2-yl)phenyl, 1-hydroxyethyl, and 3-aminosulfonylphenyl;
(g) when R^{4a} is cyano, then R^{3a} is not selected from pyridin-4-ylmethoxy, pyridin-2-ylmethoxy, 6-methoxypyridin-2-ylmethoxy;
(h) when R^{4a} is cyclopropyl, then R^{3a} is not selected from azetidin-3-yloxy; and
(i) when R^{4a} is hydrogen, then Ar^{1a} is 3-(aminosulfonyl)phenyl.

In some aspects, the present invention provides compounds of Formula (IIa):

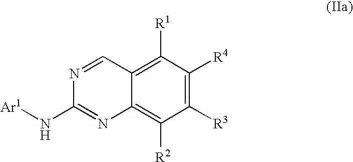

or pharmaceutically acceptable salts thereof, wherein:
R¹ is selected from H and halogen
R² is selected from H, halogen, and C₁₋₆ alkoxy;
R³ is selected from H, —OCH₂-phenyl, —O—CH₂-Het, —OCH₂—CH₂-Het, and —O—Hy; wherein Het is 6-membered heteroaryl which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and phenyl is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl;
R⁴ is selected from cyano, halogen, C₁₋₆ alkyl, C₂₋₆ alkynyl, C₃₋₆ cycloalkyl, a thiazole ring, a pyrazole ring, and a pyridine ring; each of which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups;
Ar¹ is a moiety of Group (A), (B), or (C):

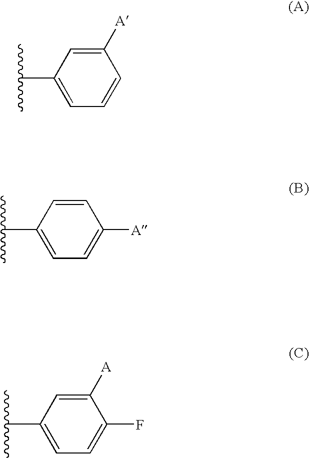

A is a pyrazole ring; which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino;
A′ is selected from -L²-Ar² and -L¹-Cy¹;
A″ is selected from L^{2a}-Ar^{2a} and —Cy^{1a};
L¹ is selected from a bond, —O—, —C(═O)—, —CH₂C(═O)—, and —CH₂—; wherein the left end of the linker is attached to the phenyl ring and the right end of the linker is attached to Cy¹;
Cy¹ is selected from a morpholine ring, a tetrahydro-2H-pyran ring, a pyrrolidine ring, a 2-oxopyrrolidine ring, and a piperidine ring, each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
Cy^{1a} is selected from a morpholine ring, a 2-oxopyrrolidine ring and a piperidine ring; each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
L² and L^{2a} are each independently selected from a bond, —O—, and —CH₂—; and
Ar² is selected from a pyrazole ring, an oxazole ring, an imidazole ring, a triazole ring, a thiadiazole ring, a pyridine ring, and a pyrimidine ring; each of which is optionally substituted with 1, 2, or 3 groups independently selected from hydroxyl, C₁₋₆ alkyl, C₁₋₄ alkoxy, amino, C₁₋₄ alkylamino, and di-C₁₋₄ alkylamino; and
Ar^{2a} is selected from a pyrazole ring, a triazole ring, a pyridine ring, and a pyrimidine ring; each of which is optionally substituted with 1 or 2 groups independently selected from hydroxyl, C₁₋₆ alkyl, C₁₋₄ alkoxy, amino, C₁₋₄ alkylamino, and di-C₁₋₄ alkylamino.

In some embodiments, the following provisos apply:
(i) when R¹ and R² are each H, R⁴ is thiazol-2-yl, R³ is optionally substituted piperidin-4-yloxy, and Ar¹ is a moiety of Group (A), then A′ is not selected from 1,2,4-triazol-1-ylmethyl, 1-methylpyrazol-3-yl, 2-oxopyrrolidinyl, oxazol-5-yl, pyrazol-1-yl, 1-methyl-1,2,4-triazol-2-yl, morpholin-4-ylcarbonylmethyl, imidazol-2-yl, 2-methylthiazol-4-yl, 1,3,5-trimethylpyrazol-4-yl, pyrimidin-5-yl, 1,2,4-triazol-1-yl, 4,5-dimethyloxazol-2-yl, pyrimidin-5-yl, 2-methoxypyrimidin-5-yl, 6-methoxypyridin-3-yl, and pyridin-3-yl;
(ii) when R¹ and R² are each H, Ar¹ is a moiety of Group (A), A′ is selected from 1-methylpyrazol-3-yl, and R³ is selected from pyridin-3-ylmethoxy and pyrazin-2-ylmethoxy, then R⁴ is not thiazol-2-yl;
(iii) when R¹ and R² are each H, R⁴ is thiazol-2-yl, R³ is optionally substituted piperidin-4-yloxy, and Ar¹ is a moiety of Group (B), then A″ is not selected from morpholin-4-yl, morpholin-4-ylcarbonylmethyl, pyrimidin-5-yl, and pyrazol-1-ylmethyl;

(iv) when R¹ and R² are each H, R⁴ is bromo, R³ is pyridin-3-ylmethyl, and Ar¹ is a moiety of Group (A), then A′ is not piperidin-4-yl;
(v) when R¹ and R³ are each H, R⁴ is bromo or ethynyl, R² is isopropoxy, and Ar¹ is a moiety of Group (A), then A′ is not selected from 1-methyl-1,2,4-triazol-2-ylmethyl and morpholin-4-ylcarbonylmethyl;
(vi) when R¹ and R² are each H, R⁴ is cyclopropyl, bromo or ethynyl, R³ is chloro, and Ar¹ is a moiety of Group (A), then A′ is not 1,2,4-triazol-1-ylmethyl;
(vii) when R¹ and R² are each H, R⁴ is ethynyl, R³ is chloro, and Ar¹ is a moiety of Group (A), then A′ is not imidazol-2-yl, oxazol-5-yl, pyrazol-1-yl, and morpholin-4-ylmethyl;
(viii) when R¹ and R³ are each H, R⁴ is ethynyl, R¹ is chloro, and Ar¹ is a moiety of Group (A), then A′ is not selected from pyrazol-1-yl, oxazol-5-yl, imidazol-2-yl, morpholin-4-ylcarbonylmethyl, and 2-oxopyrrolidinyl;
(ix) when R¹ and R² are each H, R⁴ is ethynyl, R³ is H, and Ar¹ is a moiety of Group (A), then A′ is not selected from 1,2,4-triazol-1-ylmethyl, pyrazol-1-yl, oxazol-5-yl, imidazol-2-yl, 5-trifluoromethylimidazol-2-yl, imidazol-1-ylmethyl, 1,2,4-triazol-1yl-methyl, N-(ethoxycarbonyl)piperidin-4-yl, N-(methoxycarbonyl)piperidin-4-yl, N-(acetyl)piperidin-4-yl, N-methylpiperidin-4-yl, piperidin-4-yl, and 2-oxopyrrolidinyl;
(x) when R¹ and R² are each H, R⁴ is ethynyl, Ar¹ is a moiety of Group (A), and A′ is 1-methylpyrazol-3-yl, then R³ is not selected from pyridin-3-ylmethoxy, 2-pyridin-3-ylethoxy, thiazol-5-ylmethoxy, and pyrazin-2-ylmethoxy;
(xi) when R¹ and R² are each H, R⁴ is ethynyl, Ar′ is a moiety of Group (A), and A′ is morpholin-4-ylmethyl, then R³ is not selected from pyridin-3-ylmethoxy and pyrazin-2-ylmethoxy;
(xii) when R¹ and R² are each H, R⁴ is ethynyl, Ar¹ is a moiety of Group (A), and A′ is oxazol-5-yl, then R³ is not selected from pyridin-3-ylmethoxy and pyrazin-2-ylmethoxy;
(xiii) when R¹ and R² are each H, R⁴ is ethynyl, R³ is H, and Ar¹ is a moiety of Group (B), then A″ is not selected from morpholin-4-ylmethyl and 2-oxopyrrolidinyl;
(xiv) when R′ and R² are each H, R⁴ is ethynyl, R³ is chloro, and Ar¹ is a moiety of Group (B), then A″ is not selected from 2-oxopyrrolidinyl and morpholin-4-ylmethyl;
(xv) when R¹ and R² are each H, R³ is pyrazin-2-ylmethoxy, R⁴ is pyrazol-4-yl, and Ar¹ is a moiety of Group (A), then A′ is not 1-methylpyrazol-3-yl;
(xvi) when R¹ and R² are each H, R³ is piperidin-4-yloxy or N-methylpiperidin-4-yloxy, R⁴ is pyrazol-4-yl, and Ar¹ is a moiety of Group (B), then A″ is not morpholin-4-yl;
(xvii) when Ar¹ is 3-morpholin-4-ylphenyl, then R³ is not H;
(xviii) when Ar¹ is 4-morpholin-4-ylphenyl, then R⁴ is not selected from bromo, cyano, ethynyl, thiazol-2-yl, and 1H-pyrazol-4-yl;
(xix) when R^{1′} is H, R^{2′} is H, R^{3′} is piperidin-4-yloxy, and R^{4′} is 6-methoxypyridin-3-yl, and Ar¹ is a moiety of Group (A), then A′ is not 6-methoxypyridin-3-yl; and
(xx) when R^{1′} is H, R^{2′} is H, R^{3′} is piperidin-4-yloxy, and R^{4′} is 5-methoxypyridin-3-yl, and Ar¹ is a moiety of Group (B), then A″ is not 5-methoxypyridin-3-yl.

In some embodiments, Hy can be 5-membered heterocycloalkyl and the following provisos further apply:

(a) when R¹ and R² are each H, R⁴ is bromo, R³ is pyrrolidin-3-yloxy, and Ar′ is a moiety of Group (A), then A′ is not selected from 1,2,4-triazol-1-ylmethyl, oxzol-5-yl, and morpholin-4-ylcarbonylmethyl;

when R¹ and R² are each H, R⁴ is thiazol-2-yl, R³ is optionally substituted pyrrolidin-4-yloxy, and Ar¹ is a moiety of Group (A), then A′ is not selected from 1,2,4-triazol-1-ylmethyl, 1-methylpyrazol-3-yl, 2-oxopyrrolidinyl, oxazol-5-yl, pyrazol-1-yl, 1-methyl-1,2,4-triazol-2-yl, morpholin-4-ylcarbonylmethyl, 2-methylthiazol-4-yl, 1,3,5-trimethylpyrazol-4-yl, pyrimidin-5-yl, 1,2,4-triazol-1-yl, 4,5-dimethyloxazol-2-yl, pyrimidin-5-yl, 2-methoxypyrimidin-5-yl, 6-methoxypyridin-3-yl, and pyridin-3-yl;

(b) when R¹ and R² are each H, R⁴ is thiazol-2-yl, R³ is optionally substituted pyrrolidin-3-yloxy, and Ar¹ is a moiety of Group (B), then A″ is not selected from morpholin-4-yl, morpholin-4-ylcarbonylmethyl, pyrimidin-5-yl, and pyrazol-1-ylmethyl;

(c) when R¹ and R² are each H, R⁴ is ethynyl, and R³ is pyrrolidin-3-yloxy or N-methylpyrrolidin-3-yl, then A′ is not selected from 1,2,4-triazol-1-ylmethyl, morpholin-4-ylcarbonylmethyl, and 1-methylpyrazol-3-yl, when Ar¹ is a moiety of Group (A); and A″ is not selected from morpholin-4-yl, when Ar¹ is a moiety of Group (B); when R¹ and R² are each H, R³ is pyrrolidin-3-yloxy or N-methylpyrrolidin-3-yloxy, R⁴ is selected from cyano, cyclopropyl and methyl and Ar′ is a moiety of Group (A), then A′ is not 1,2,4-triazol-1-ylmethyl; and

(d) when R¹ and R² are each H, R³ is pyrrolidin-3-yloxy, R⁴ is cyclopropyl, and Ar¹ is a moiety of Group (B), then A″ is not morpholin-4-yl;

when R¹ and R² are each H, R³ is pyrrolidin-3-yloxy, R⁴ is H, and Ar¹ is a moiety of Group (B), then A″ is not morpholin-4-yl.

In another aspect, the present invention provides compounds of Formula (IIIa) or (IVa):

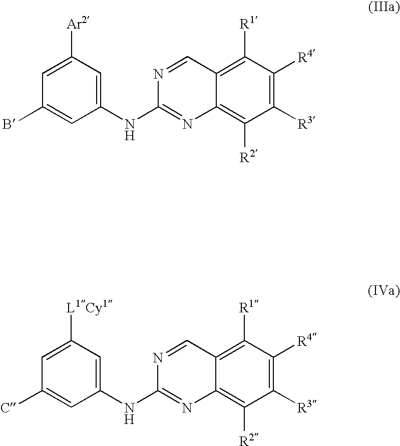

or pharmaceutically acceptable salts thereof, wherein:

R^{1′} and R^{1″} are each independently selected from H and halogen;

R^{2′} and R^{2″} are each independently selected from H, halogen, and C₁₋₆ alkoxy;

R^{3′} and R^{3″} are each independently selected from H, halogen, and C₃₋₅ heterocycloalkyloxy;

R^{4′} and R^{4″} are each independently selected from H, halogen, cyano, C₁₋₆ alkyl, C₂₋₆ alkynyl, C₃₋₆ cycloalkyl, 5-membered heteroaryl and 6-membered heteroaryl; wherein said C₃₋₆ cycloalkyl, 5-membered heteroaryl and 6-membered heteroaryl are each optionally substituted by 1 or 2 groups independently selected from C₁₋₆ alkyl and C₁₋₆ alkoxy;

B′ is selected from -L^{1′}Cy^{1′} and B″;

B″ is selected from C₁₋₆ alkyl and C₁₋₆ alkoxy; which are each substituted by 1, 2, or 3 groups independently selected from hydroxyl, amino, C₁₋₆ alkylamino, di-C₁₋₆ alkylamino, and C₁₋₆ alkylcarbonylamino; wherein said C₁₋₆ alkylamino, di-C₁₋₆ alkylamino, and C₁₋₆ alkylcarbonylamino are each optionally substituted by 1 or 2 groups independently selected from C₁₋₄ alkoxy, amino, C₁₋₄ alkylamino, and di-C₁₋₄ alkylamino;

L^{1′} is selected from —OCH₂CH₂—, —C(═O)—, —OCH₂CH₂NHC(═O)—, —OCH₂CH₂NHC(═O)CH₂—, —O—, —CH₂— and —CH(CH₃)—, wherein the right end of the linker is attached to Cy^{1′};

Cy^{1′} is selected from C₃₋₅ heterocycloalkyl, which is optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl;

A^{2′} is selected from an oxazole ring, a pyrazole ring, a pyrimidine ring, and a pyridine ring, each of which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino;

L^{1″} is —CH₂—;

Cy^{1″} is selected from 5-membered heterocycloalkyl and 6-membered heterocycloalkyl; and

C″ is carbamyl.

In some embodiments, the following provisos apply to compounds of Formula (IIa):

(i) when R¹ is H, R² is H, R³ is H, Ar^{2′} is 1-methyl-1H-pyrazole, and B′ is selected from morpholin-4-ylmethyl, 2-aminoethoxy, 2-amino-3-hydroxylpropoxy, and 2-dimethylaminoethoxy, then R⁴ is not bromo;

(ii) when Ar^{2′} is 1-methyl-1H-pyrazol-4-yl, B′ is B″, B″ is other than substituted C₁₋₆ alkyl, and R^{4′} is ethynyl, then at least one of R¹, R², or R³ is not H;

(iii) when R^{1′} is H, R^{2′} is H, R^{3′} is H, R^{4′} is ethynyl, and Ar^{2′} is pyrimidin-5-yl, then B′ is not B″ or is not —OCH₂CH₂NHC(═O)—;

(iv) when R^{1′} is H, R^{2′} is H, R^{3′} is H, R^{4′} is ethynyl, and Ar^{2′} is 1-methyl-1H-pyrazol-4-yl, then B′ is not selected from 2-(pyrrolidinyl)ethoxy, 2-(4-methylpiperazinyl)ethoxy, morpholin-4-ylmethyl, 2-(methoxycarbonyl)pyrrolidin-3-yloxy, and pyrrolidin-3-yloxy;

(v) when R^{1′} is H, R^{2′} is H, R^{3′} is H, R^{4′} is ethynyl, L^{1′} is —OCH₂CH₂NHC(═O)CH₂— or —OCH₂CH₂NHC(═O)—, then Cy¹ is not selected from tetrahydro-2H-pyran-4-yl, a pyrrolidine ring optionally substituted with 1 or 2 groups independently selected from methyl, fluoro, and hydroxyl group, a piperidine ring optionally substituted with 1 or 2 groups independently selected from methyl, fluoro, and hydroxyl group, tetrahydrofuran-2-yl, and morpholin-4-yl, and Ar^{2′} is 1-methyl-1H-pyrazol-4-yl;

(vi) when R^{1′} is H, R^{2′} is H, R^{3′} is piperidin-4-yloxy, then Ar^{2′} is not oxazol-4-yl;

(vii) when R^{1′} is H, R^{2′} is H, R^{3′} is H, then B′ is morpholin-4-yl, and Ar^{2′} is pyridin-3-yl, pyridin-4-yl, 3-fluoropyridin-4-yl, and 1H-pyrazol-4-yl, then R^{4′} is not ethynyl; and

(viii) when -L^{1″}Cy^{1″} is morpholin-4-ylmethyl and C″ is carbamyl, then R⁴ is not bromo.

In another aspect, the present invention provides pharmaceutical compositions comprising a compound of the invention, or pharmaceutically acceptable salt thereof, and a pharmaceutically acceptable carrier.

In another aspect, the present invention provides methods of treating a cancer selected from lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, pharyngeal cancer, laryngeal cancer, small intestinal cancer, non-Hodgkin lymphoma, and villous colon adenoma in a patient in need thereof, comprising administering to said patient a therapeutically effective amount of a compound of the invention or pharmaceutically acceptable salt thereof.

In a further aspect, the present invention provides methods of inhibiting the tumor growth in a patient in need thereof, comprising administering to said patient a therapeutically effective amount of a compound of the invention, or pharmaceutically acceptable salt thereof.

In another aspect, the present invention provides methods of treating a disease is selected from neuro-fibromatosis, atherosclerosis, pulmonary fibrosis, arthritis, psoriasis, glomerulonephritis, restenosis, proliferative diabetic retinopathy, hypertrophic scar formation, inflammatory bowel disease, transplantation rejection, angiogenesis and endotoxic shock in a patient in need thereof, comprising administering to said patient a therapeutically effective amount of a compound of the invention, or pharmaceutically acceptable salt thereof.

In a further aspect, the present invention provides methods of inhibiting PDK1 or a PDK1 variant in a patient comprising administering to said patient, an effective amount of a compound of the invention, or pharmaceutically acceptable salt thereof.

In a further aspect, the present invention provides kits for treating a cancer selected from lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, pharyngeal cancer, laryngeal cancer, small intestinal cancer, non-Hodgkin lymphoma, and villous colon adenoma in a patient in need thereof, comprising a compound according of the invention, or pharmaceutically acceptable salt thereof, and instructions comprising a direction to administer a therapeutically effective amount of said compound, or pharmaceutically acceptable salt thereof, to said patient.

In a further aspect, the present invention provides kits for treating a disease selected from neuro-fibromatosis, atherosclerosis, pulmonary fibrosis, arthritis, psoriasis, glomerulonephritis, restenosis, proliferative diabetic retinopathy, hypertrophic scar formation, inflammatory bowel disease, transplantation rejection, angiogenesis and endotoxic shock in a patient in need thereof, comprising a compound of the invention, or pharmaceutically acceptable salt thereof, and instructions comprising a direction to administer a therapeutically effective amount of said compound, or pharmaceutically acceptable salt thereof, to said patient.

In a further aspect, the present invention provides kits for inhibiting the tumor growth in a patient in need thereof, comprising a compound of the invention, or pharmaceutically acceptable salt thereof, and instructions comprising a direction to administer a therapeutically effective amount of said compound, or pharmaceutically acceptable salt thereof, to said patient.

In a further aspect, the present invention provides kits for inhibiting PDK1 or a PDK1 variant in a patient in need thereof, comprising a compound of the invention, or pharmaceutically acceptable salt thereof, and instructions comprising a direction to administer an effective amount of said compound, or pharmaceutically acceptable salt thereof, to said patient.

The present invention further provides a use of the compounds of the invention for the preparation of medicament for use in treating the cancers and diseases described herein as well as for inhibiting tumor and inhibiting PDK1 or a PDK1 variant in an individual.

The present invention further provides a compound of the invention for use in method of treatment of the cancers and diseases described herein as well as for inhibiting tumor and inhibiting PDK1 or a PDK1 variant in an individual.

### DESCRIPTION OF DRAWINGS

FIG. 1 depicts the compounds of PCT/US2007/088392 (Table 2).

FIG. 2 depicts the compounds of PCT/US2007/088392 (Table 3).

FIG. 3 depicts the compounds of PCT/US2007/088392 (Table 4).

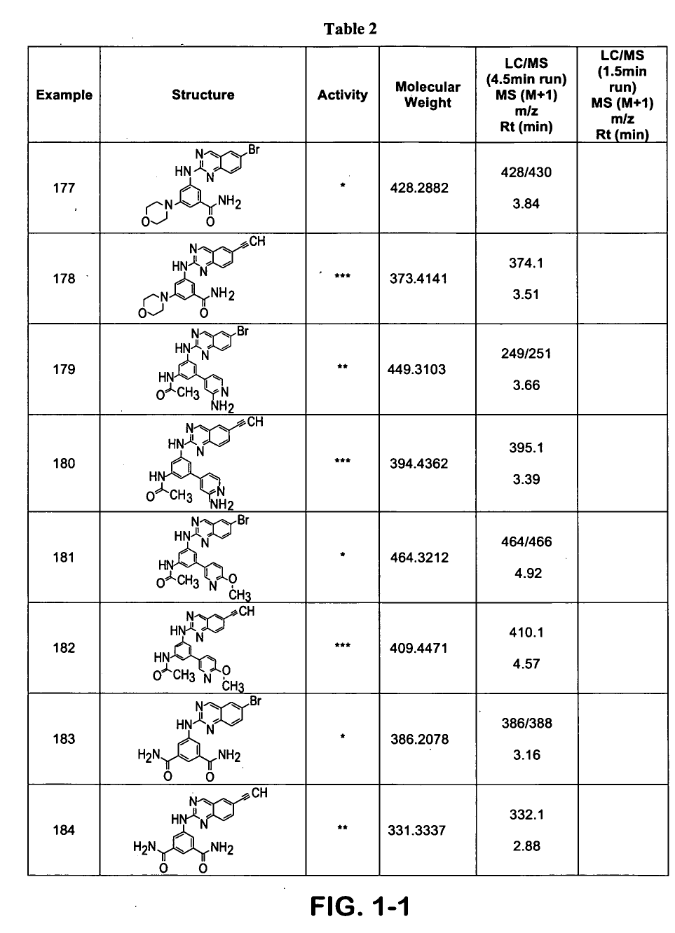

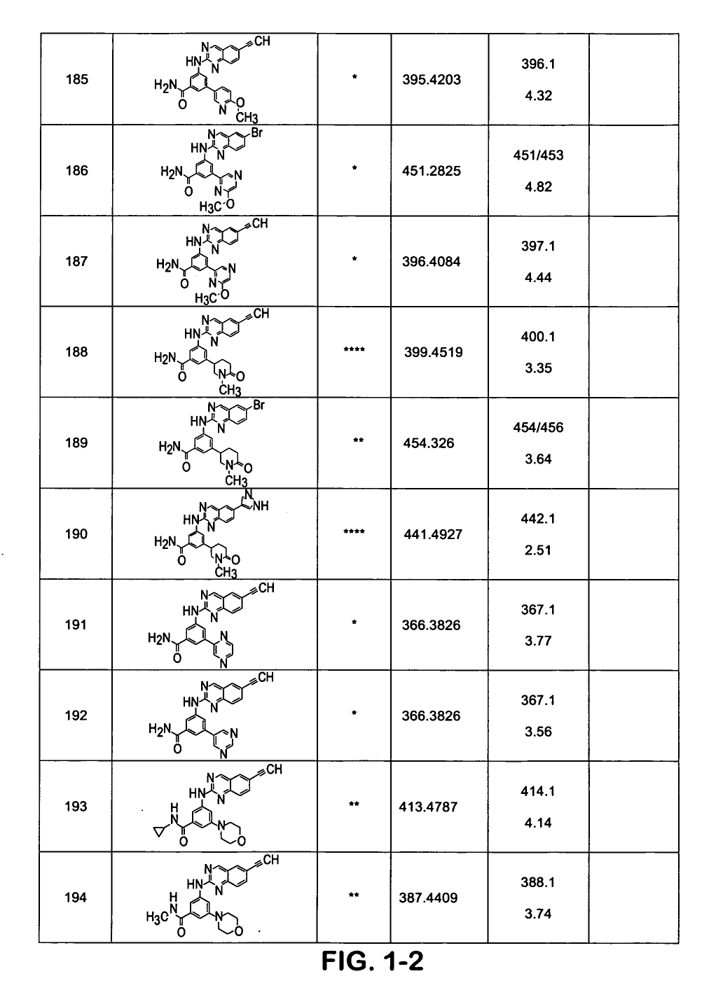

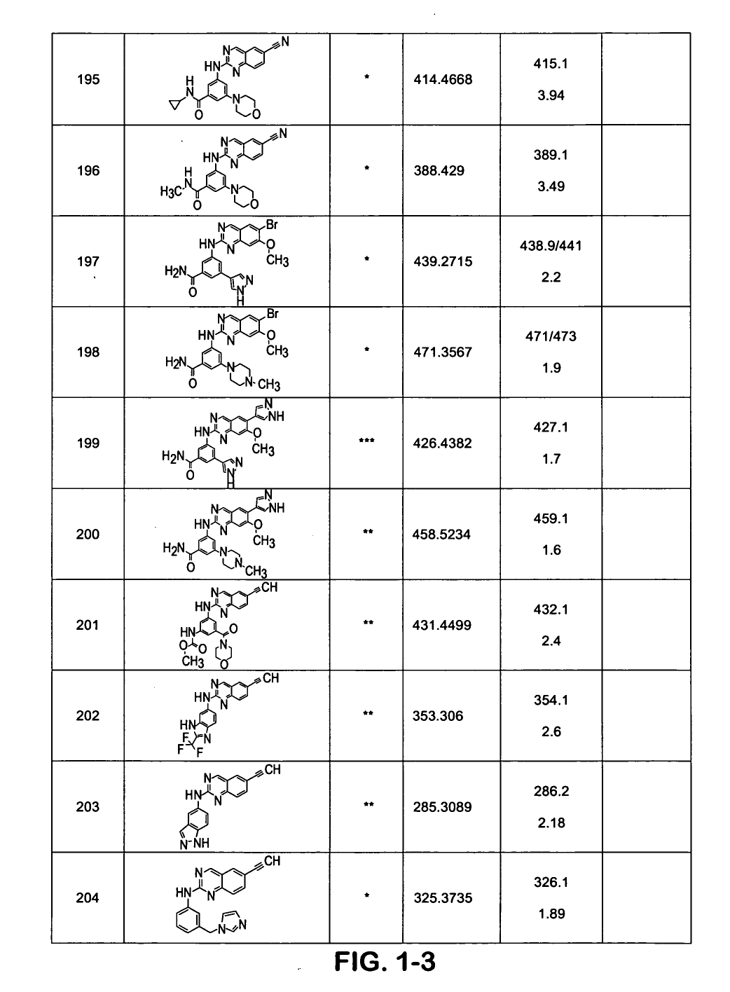

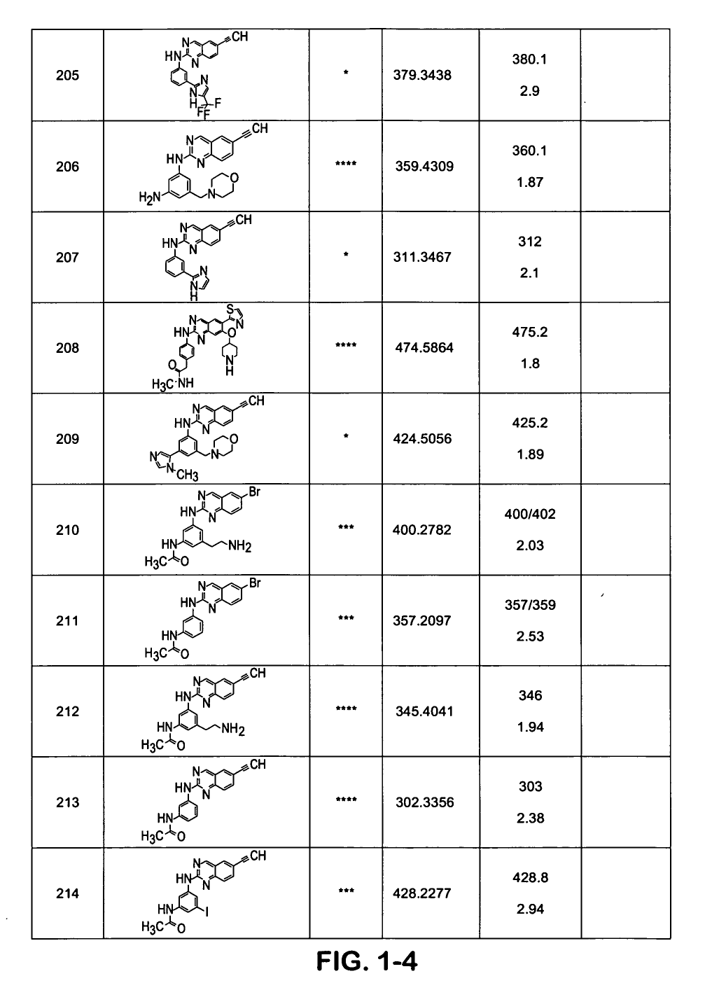

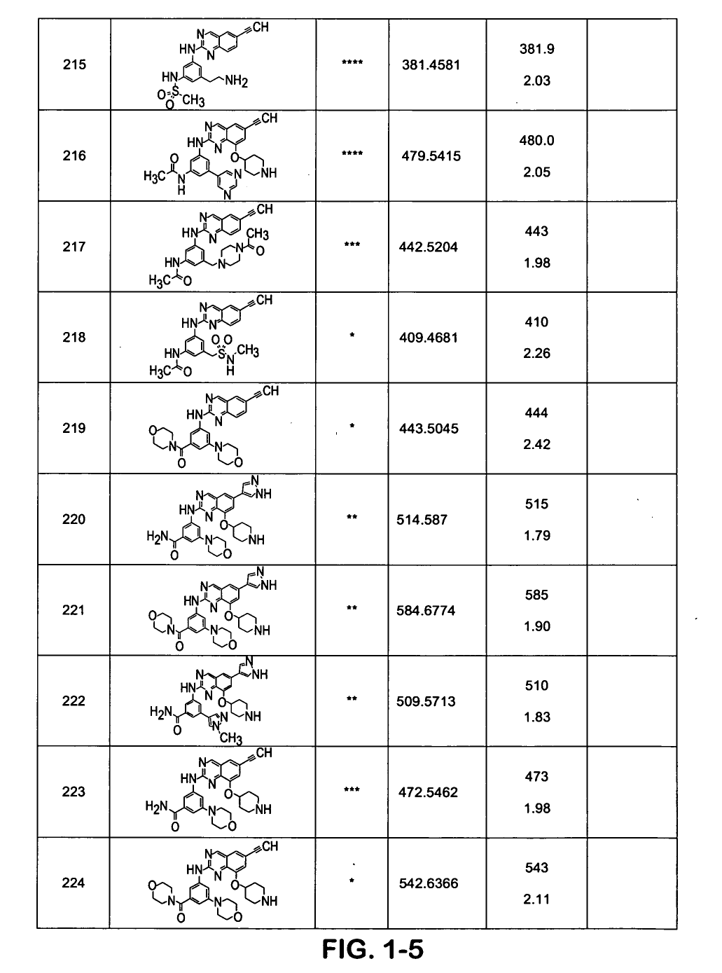

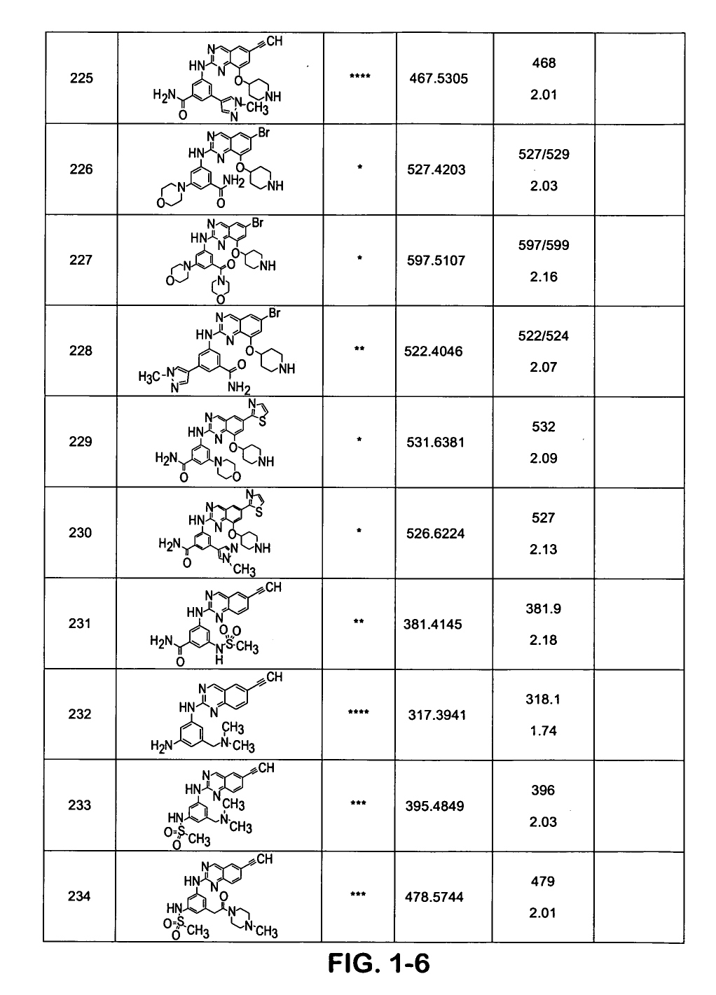

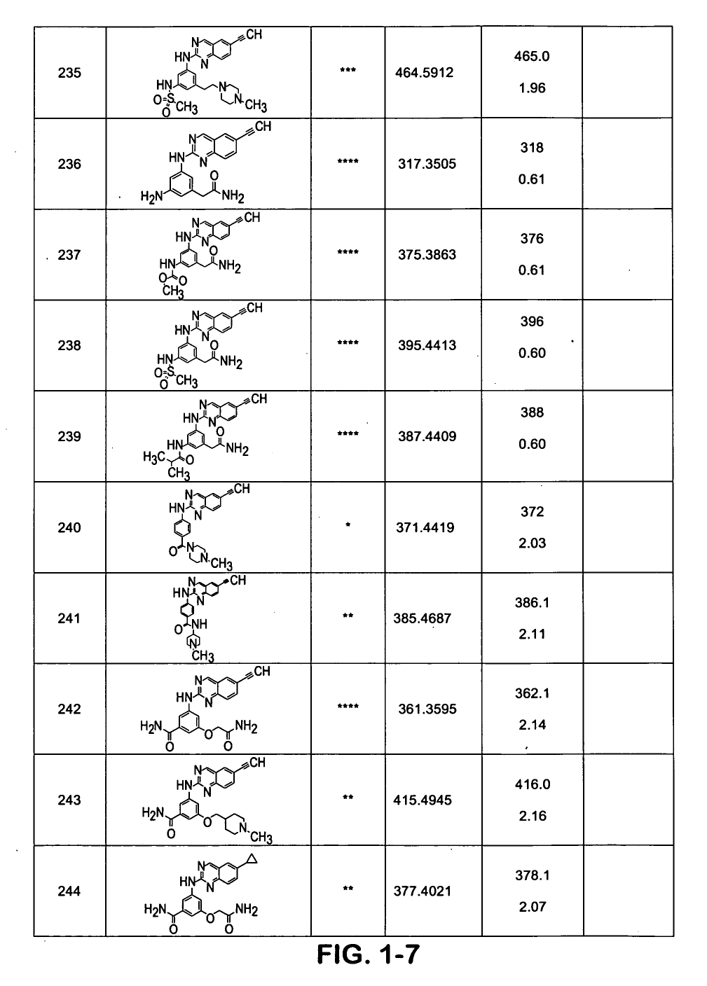

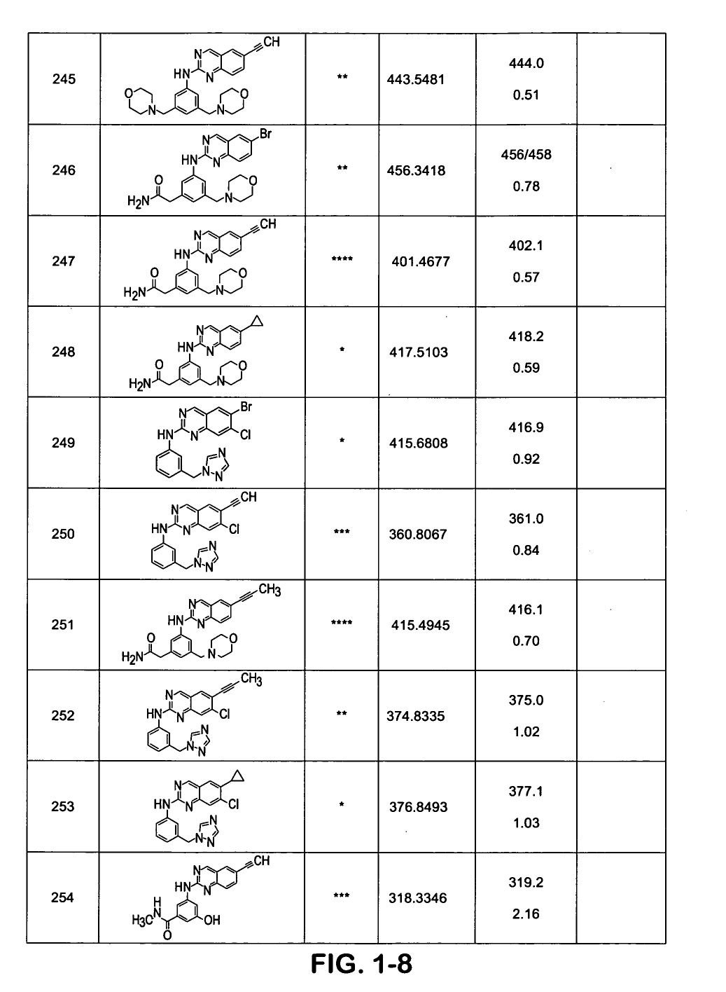

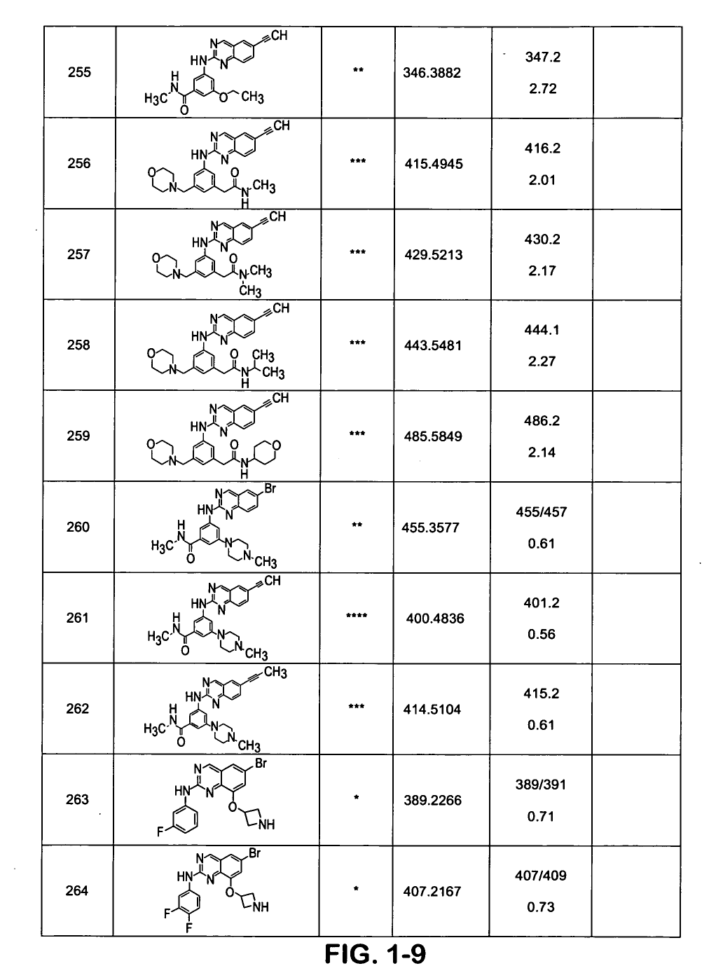

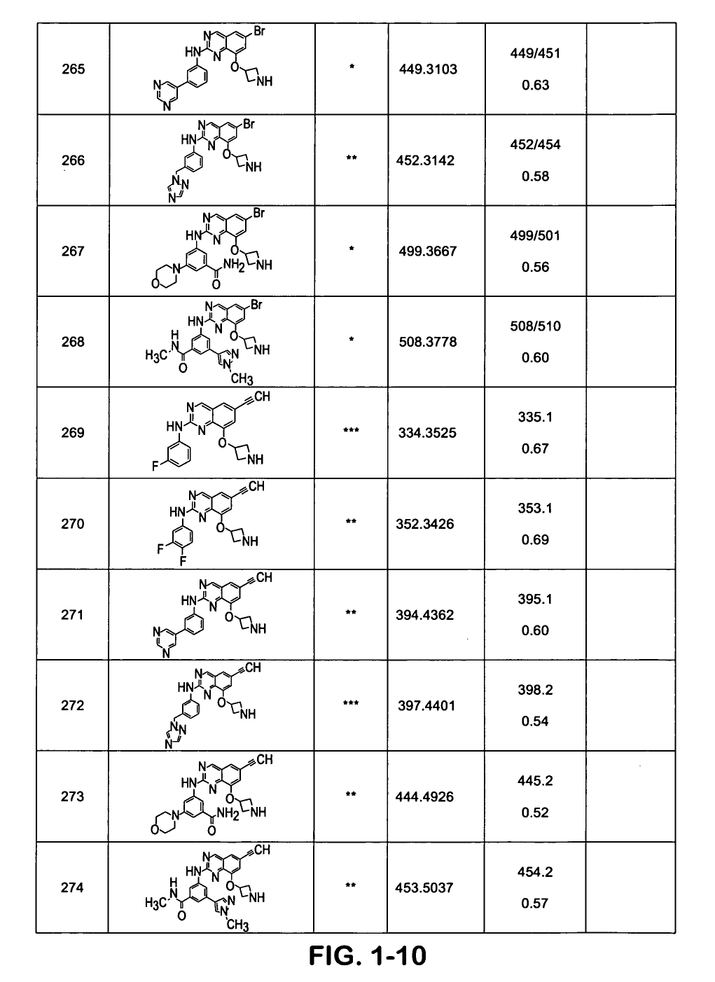

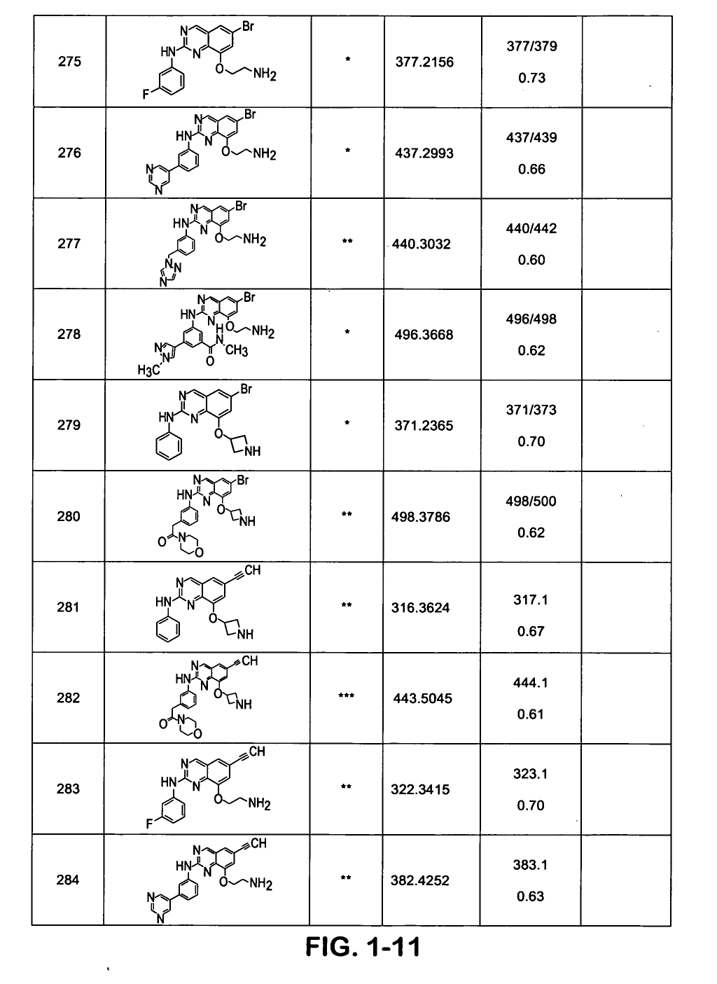

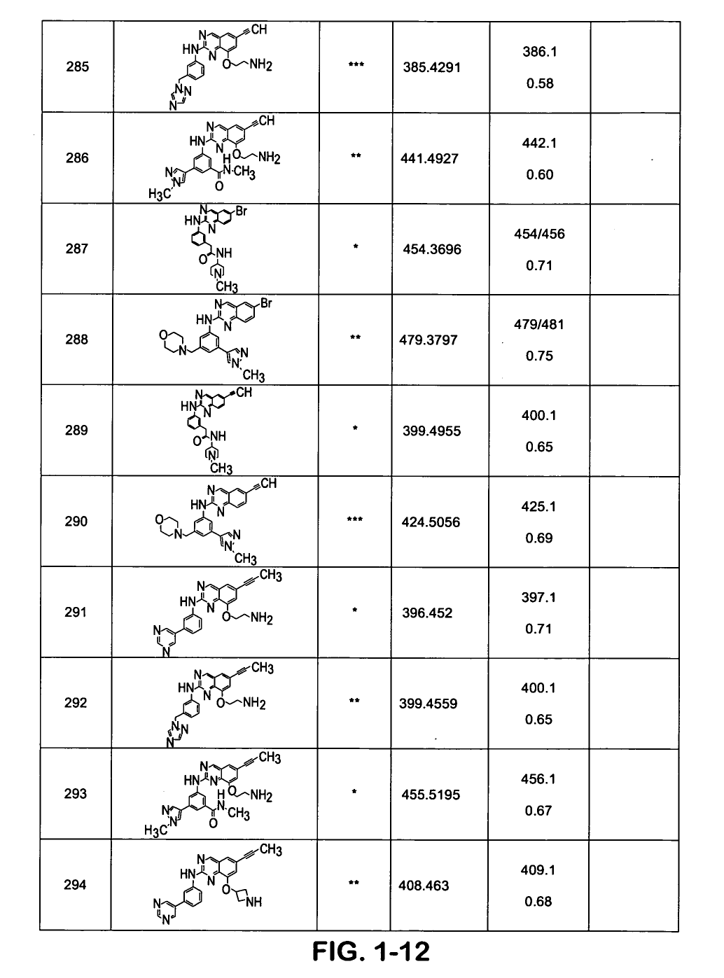

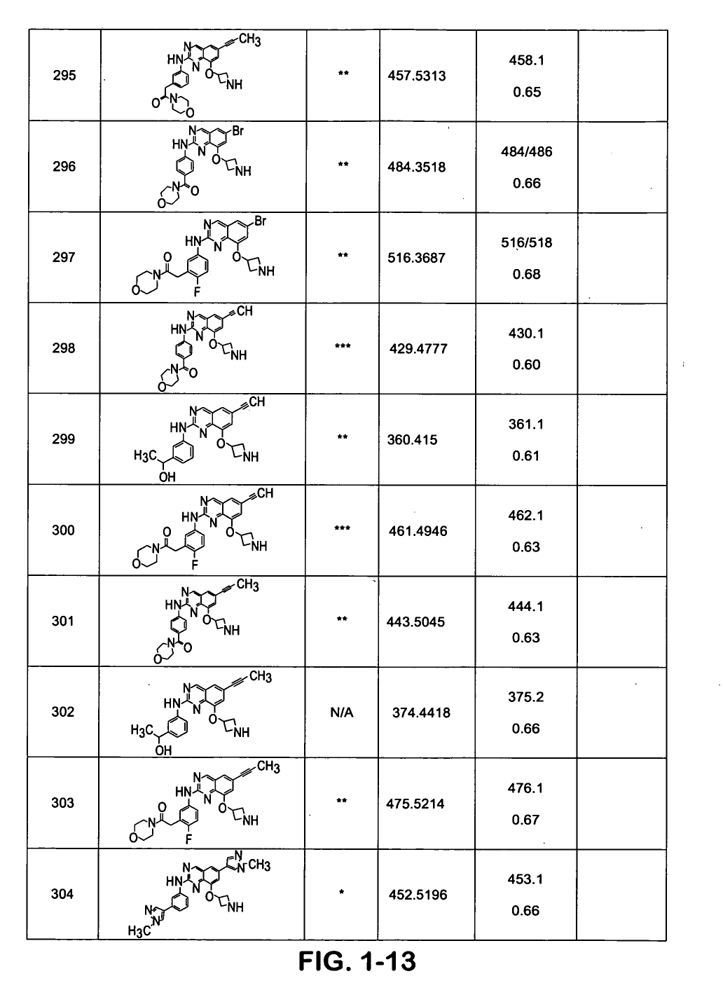

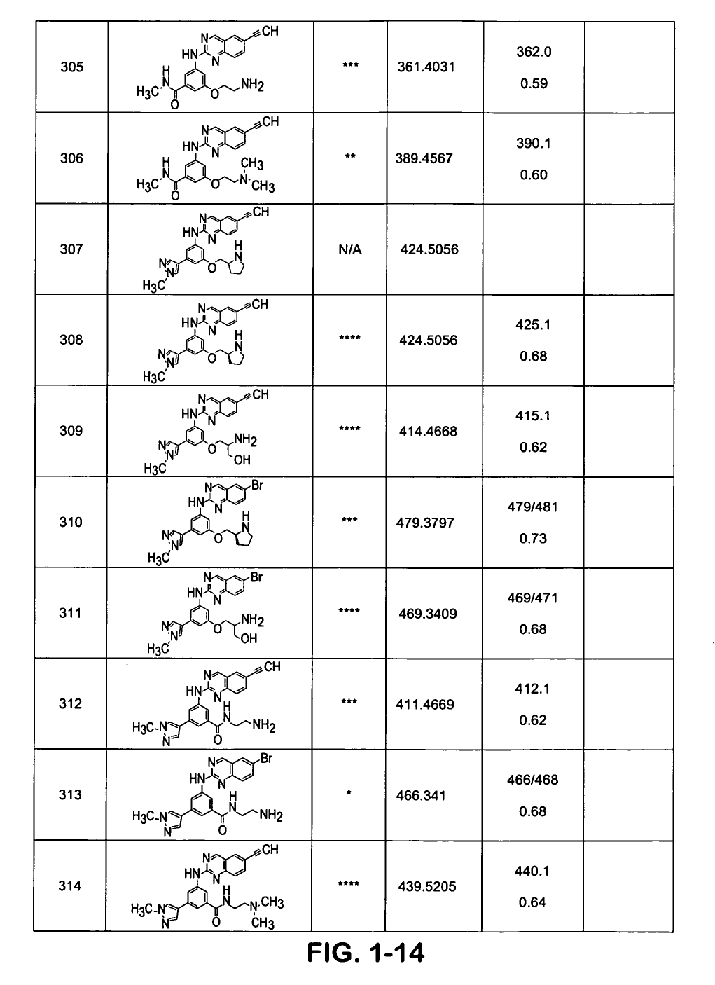

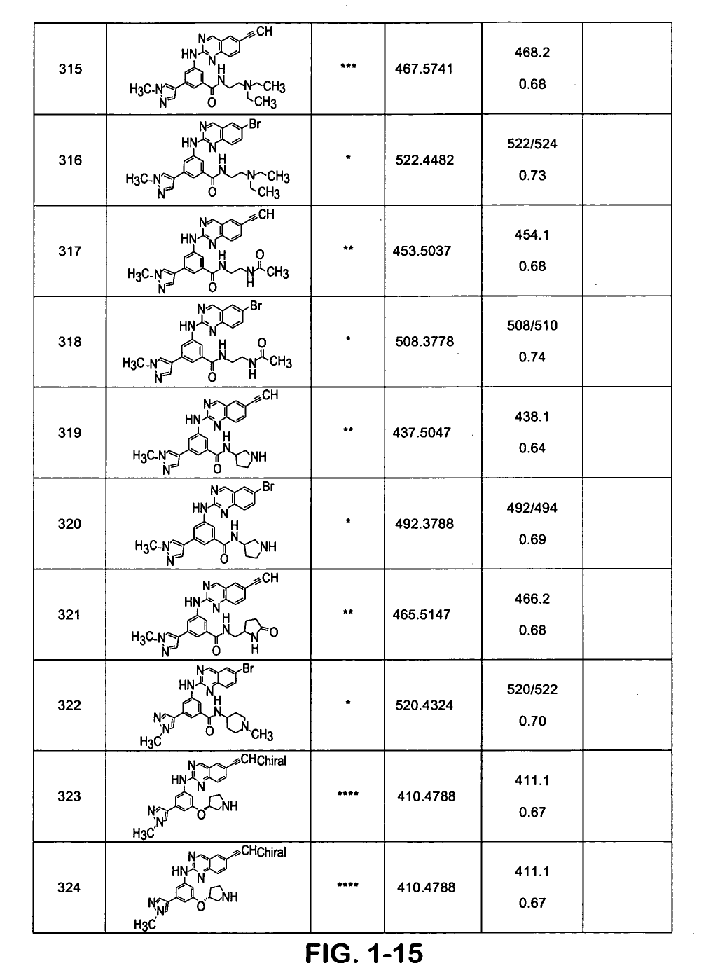

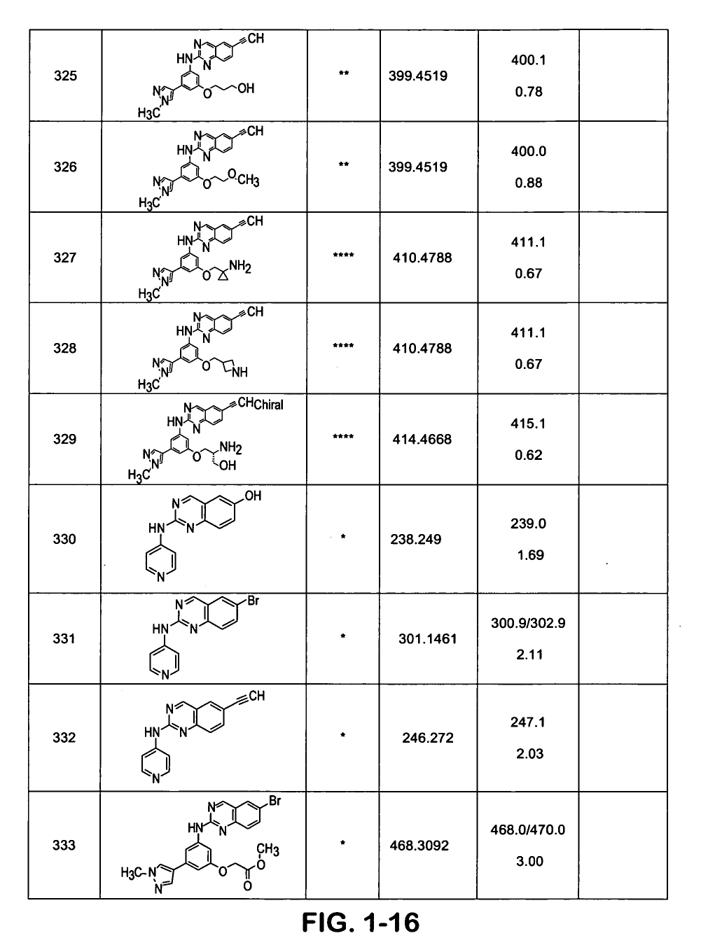

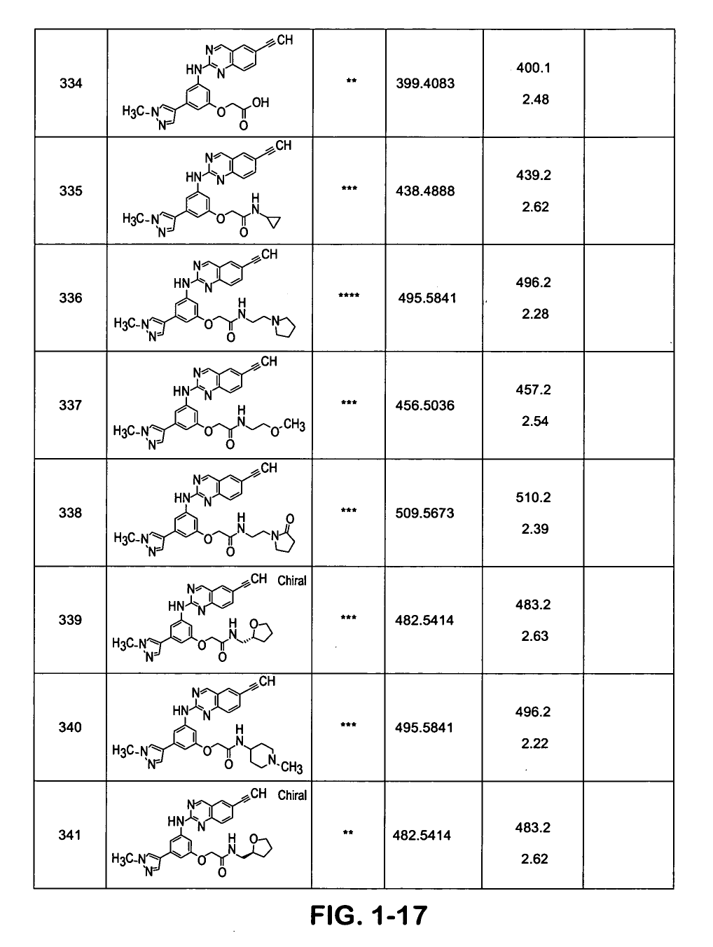

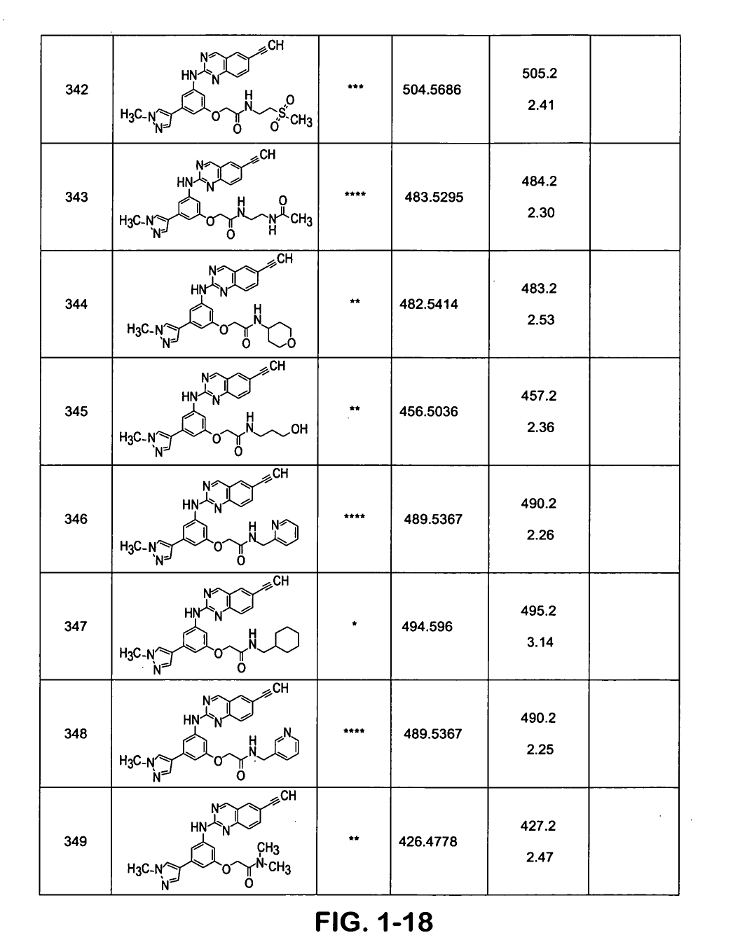

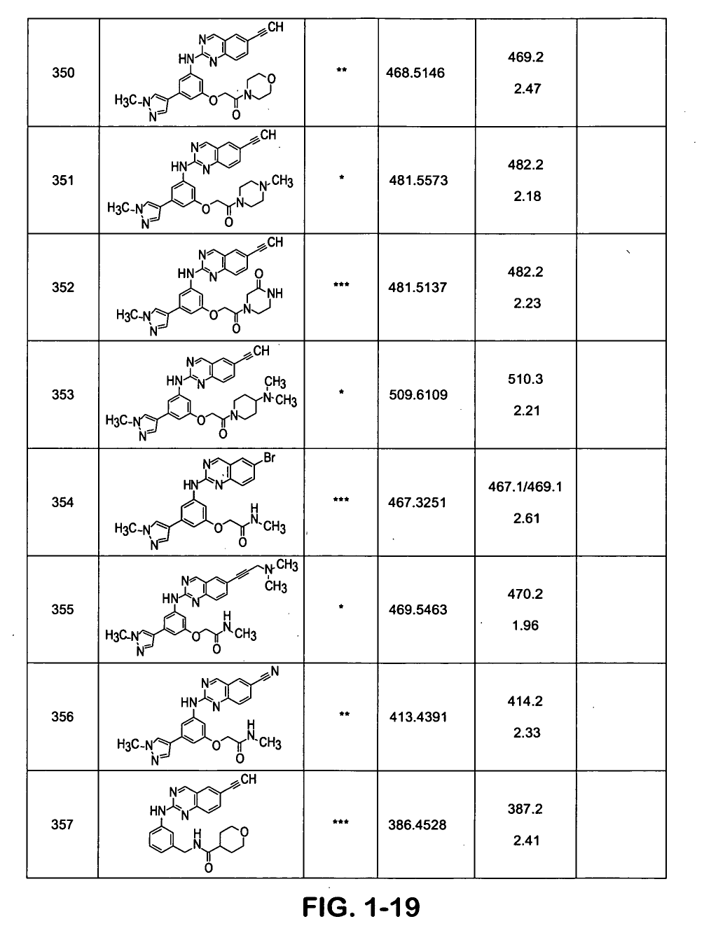

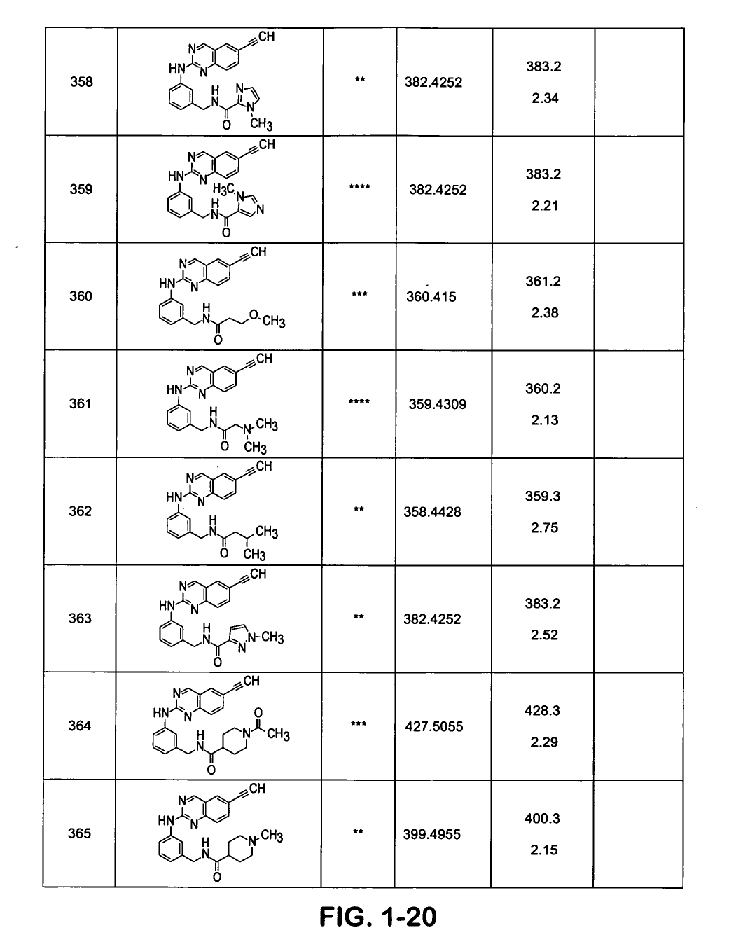

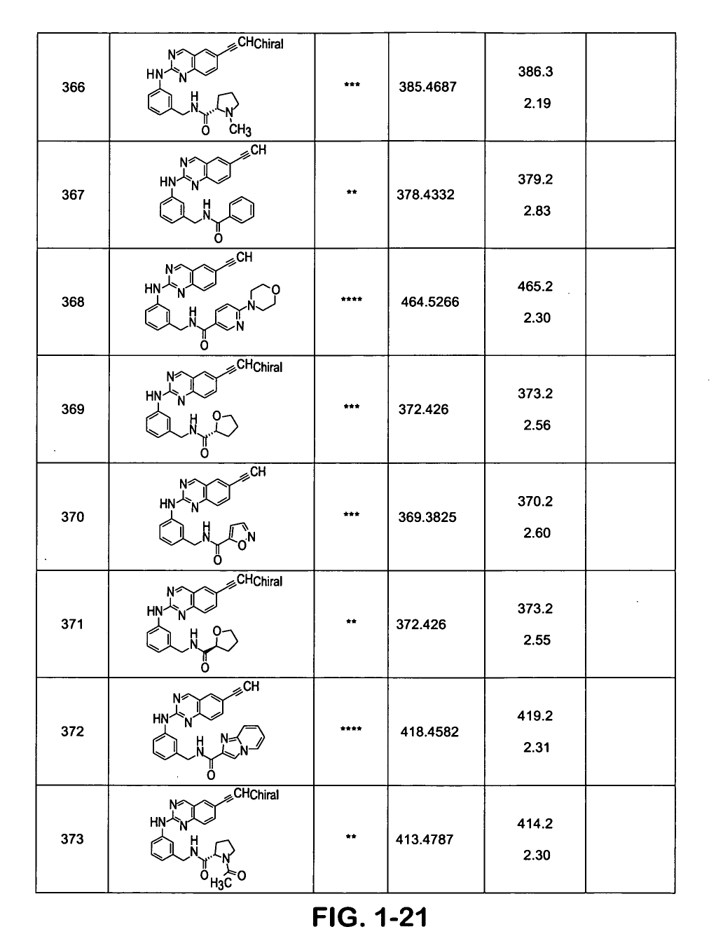

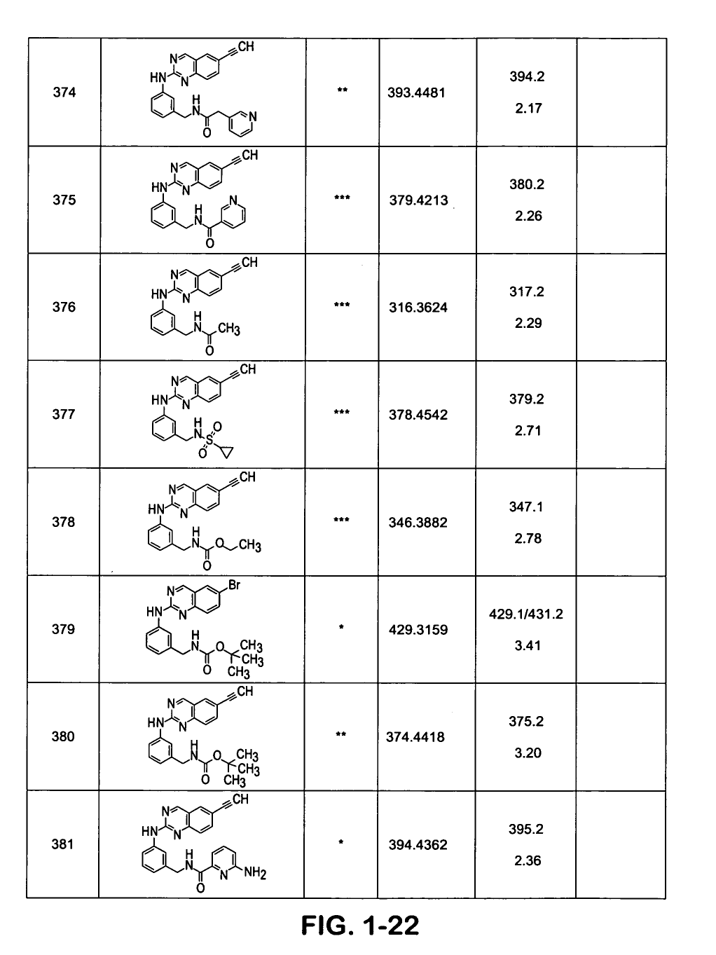

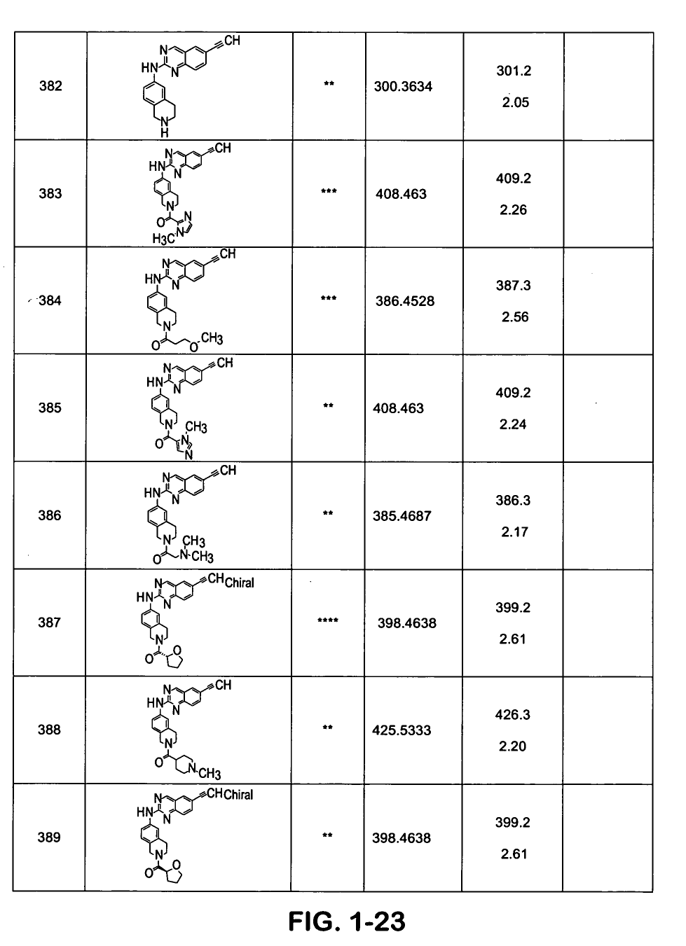

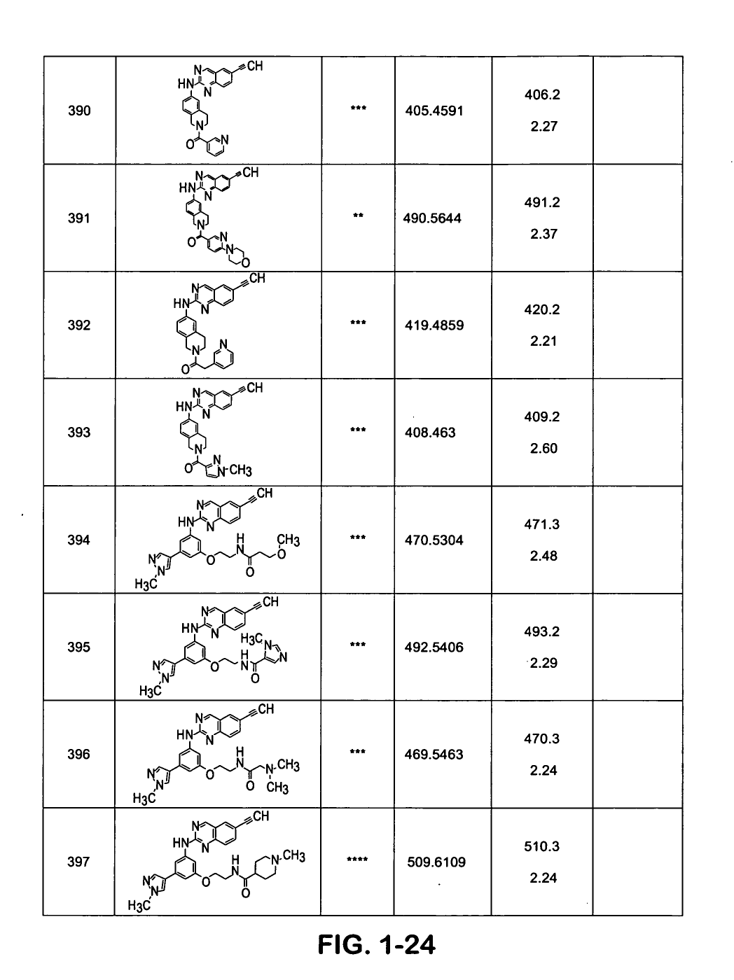

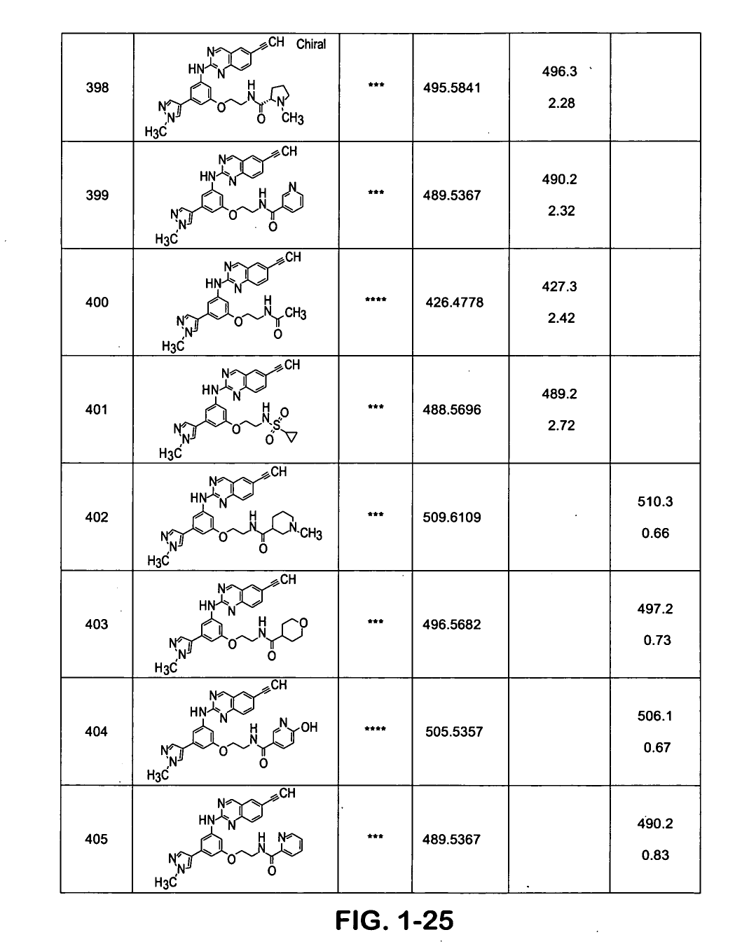

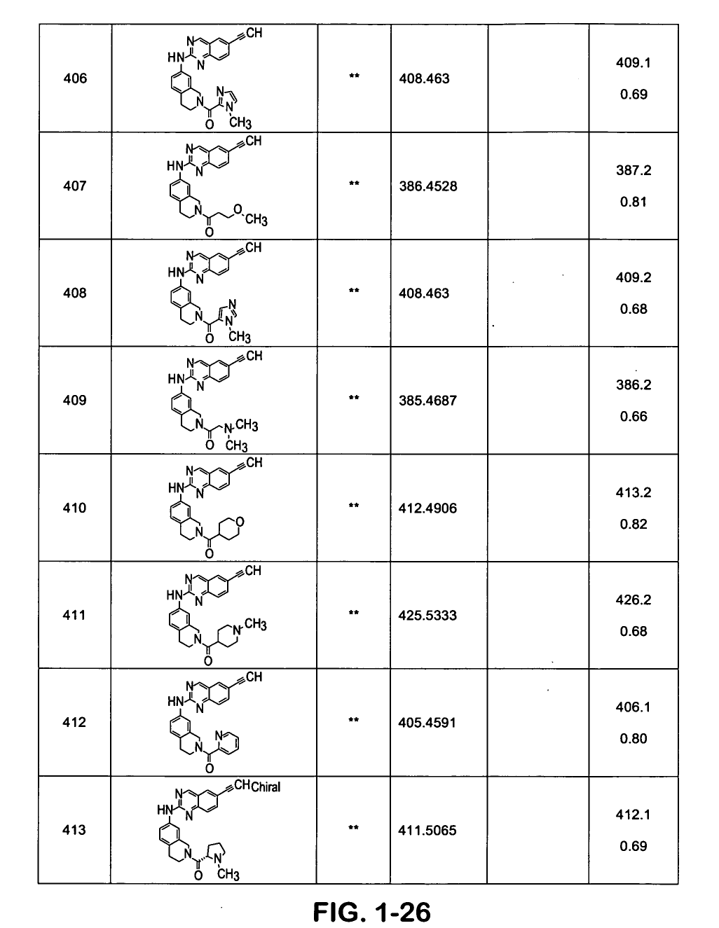

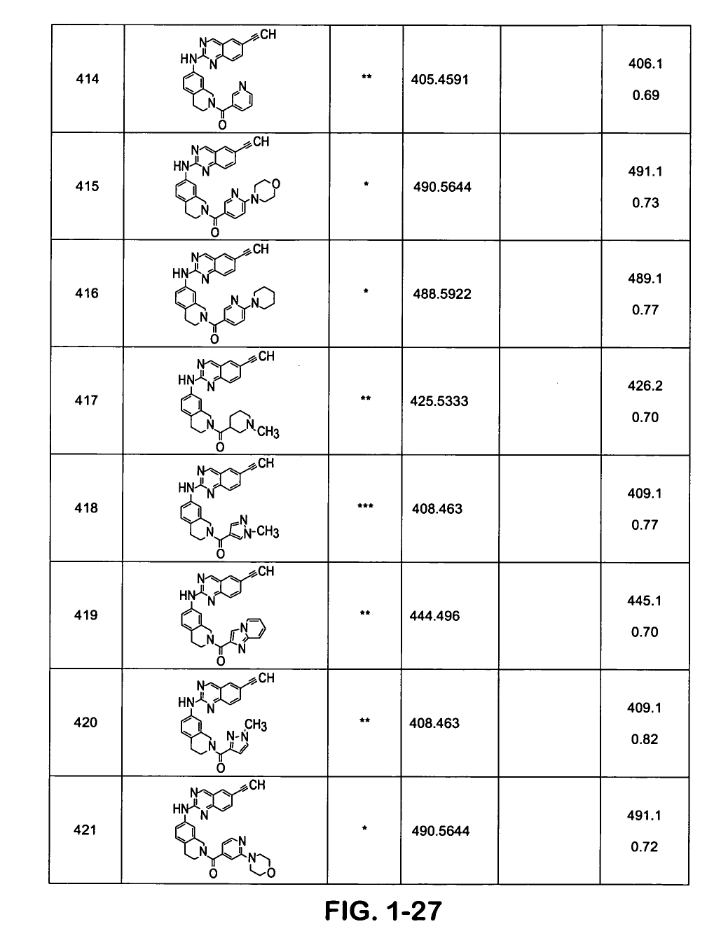

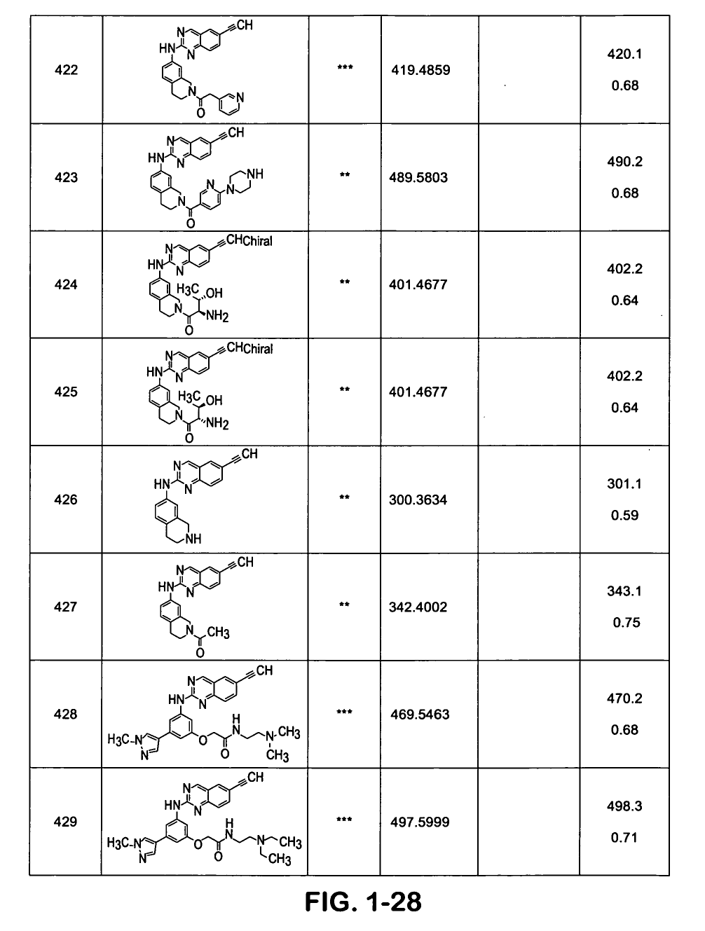

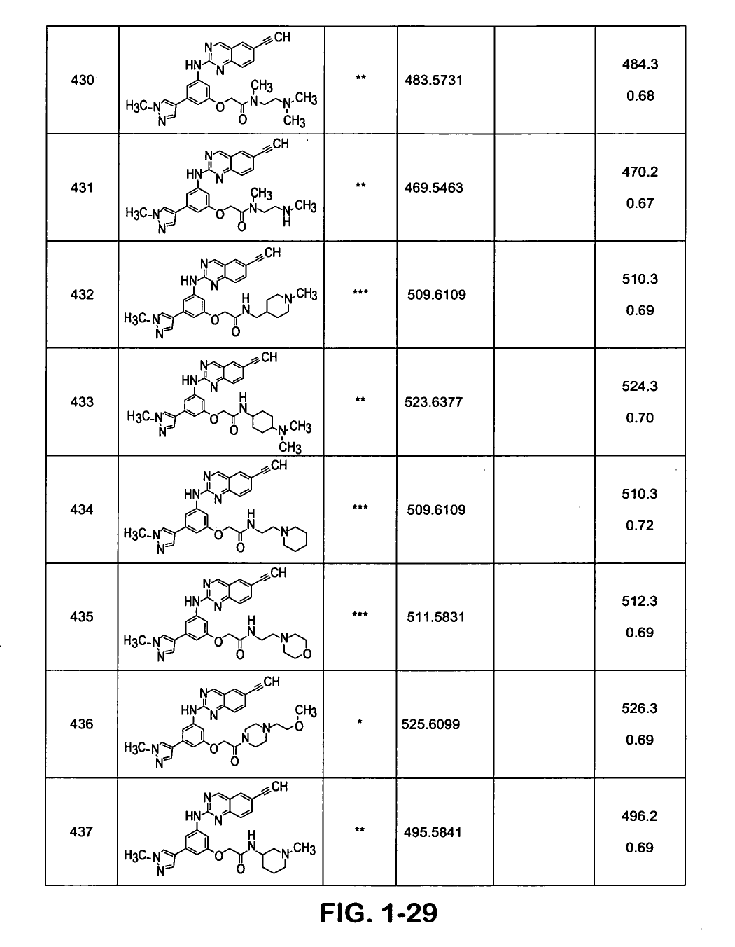

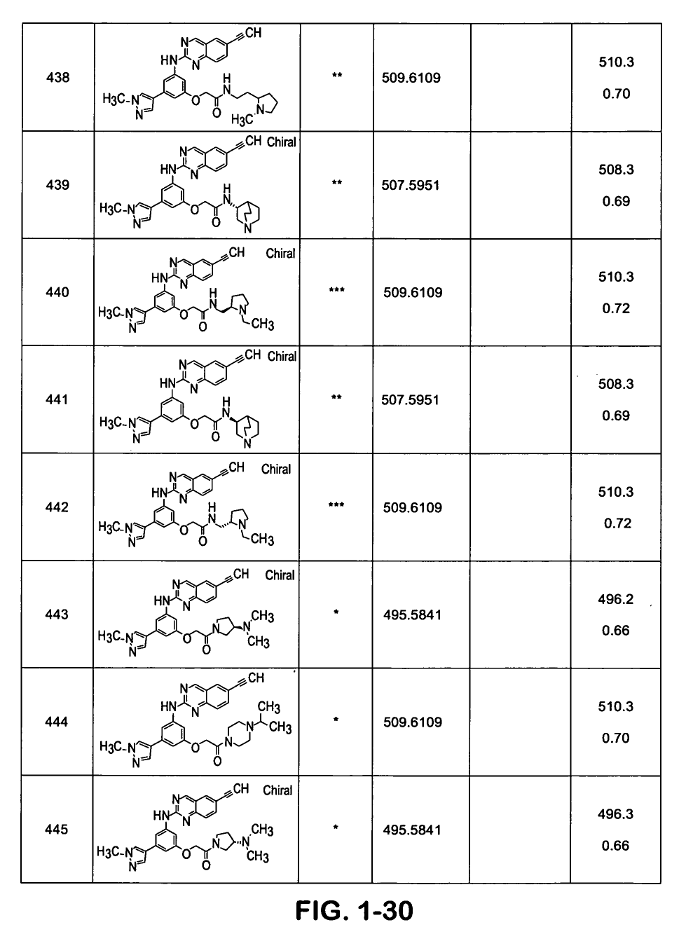

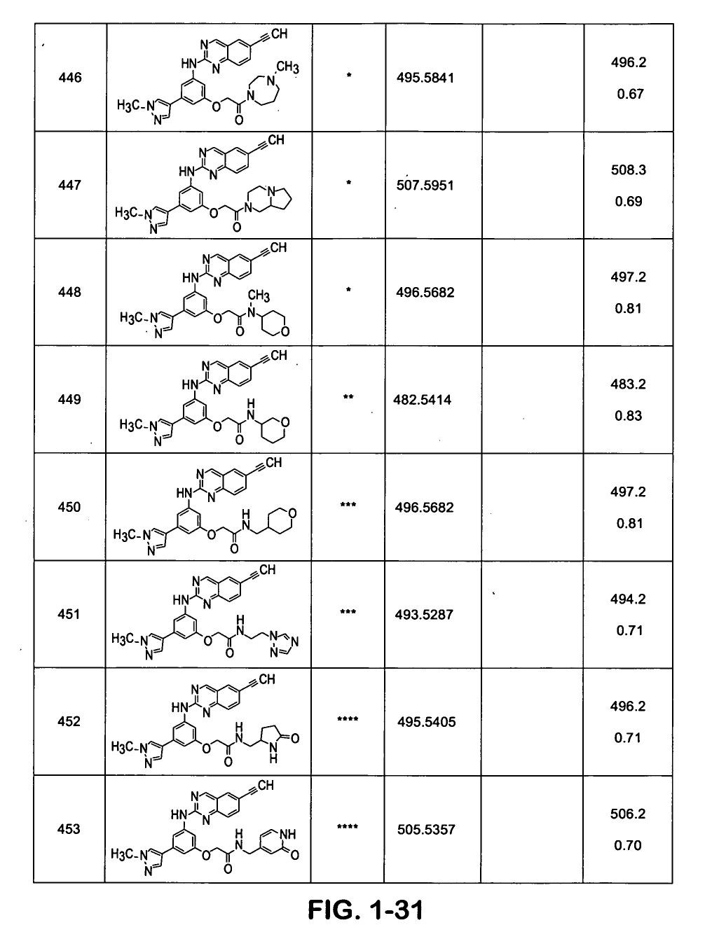

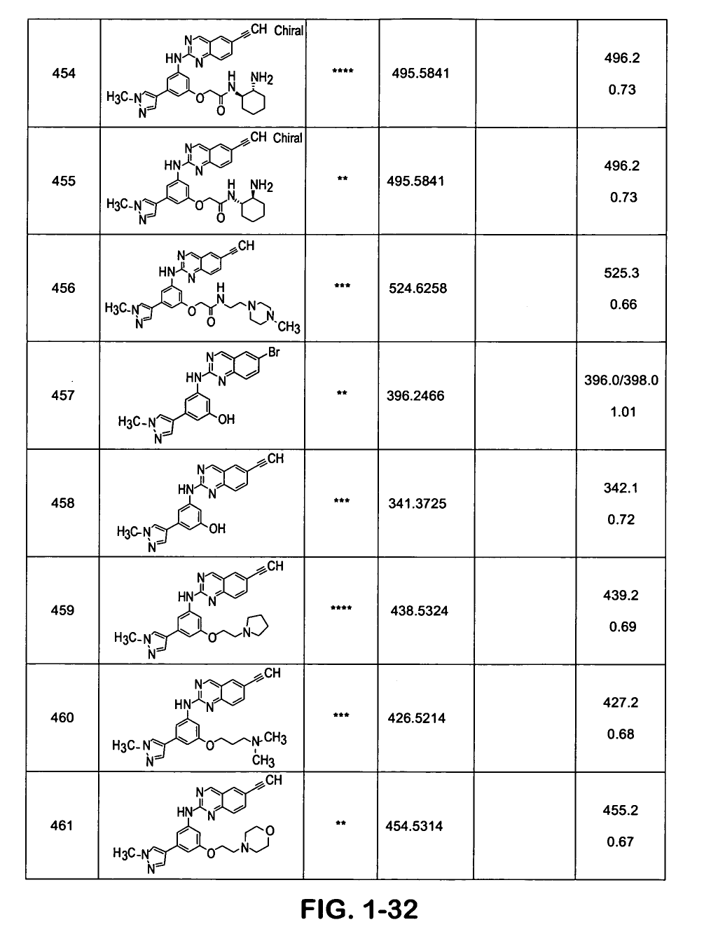

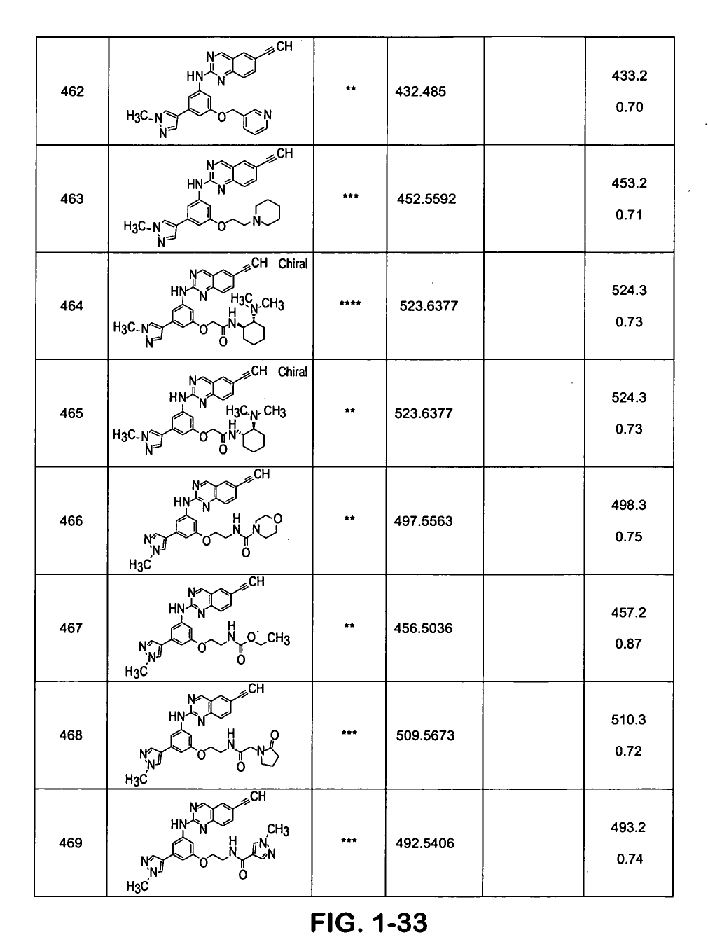

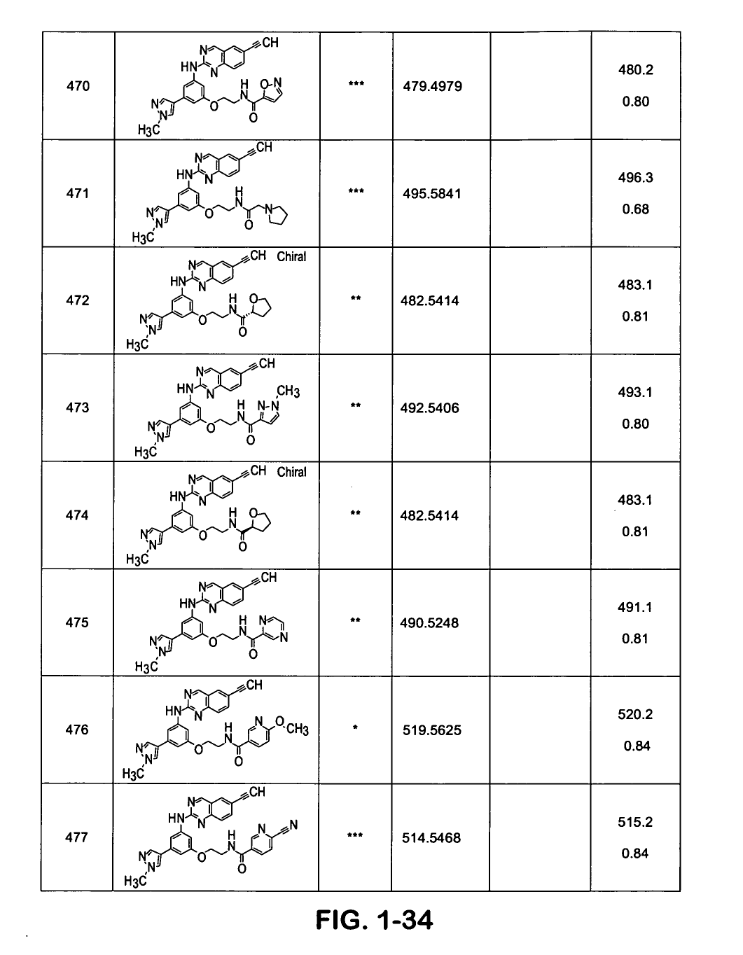

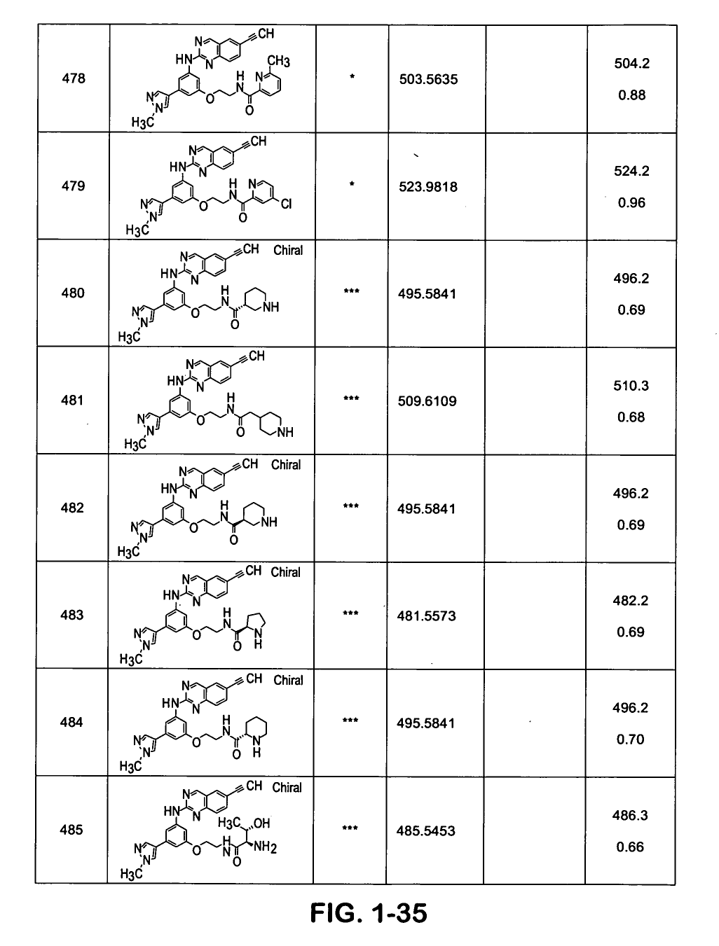

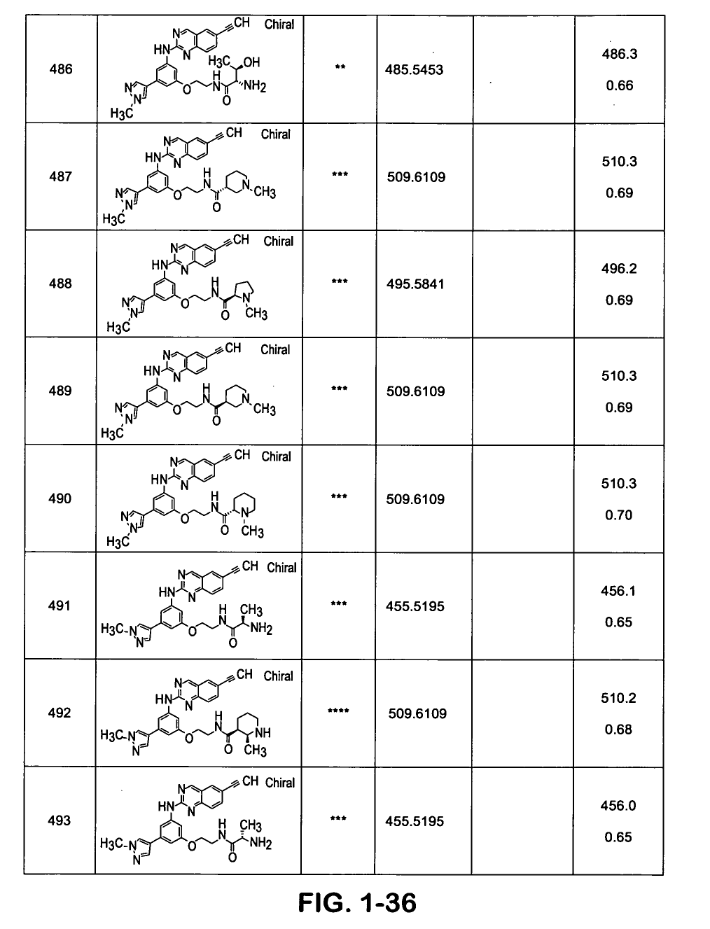

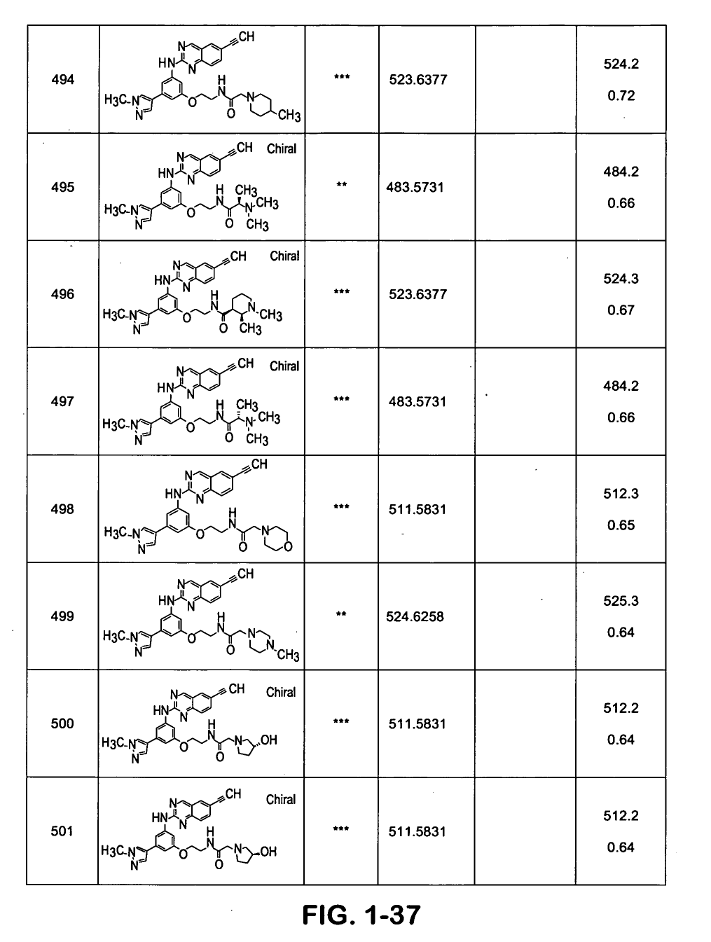

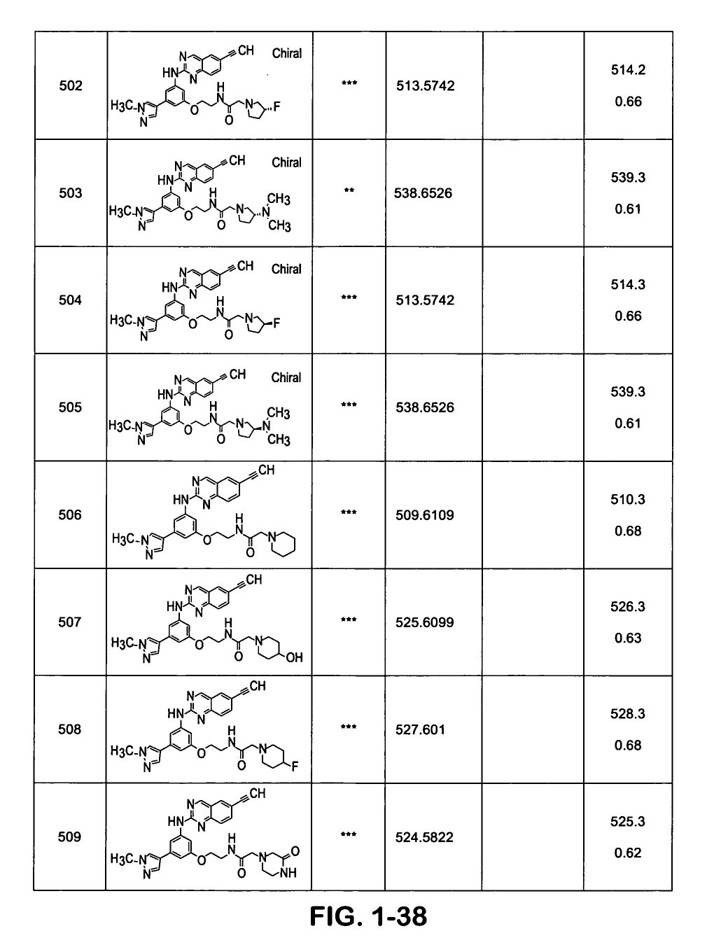

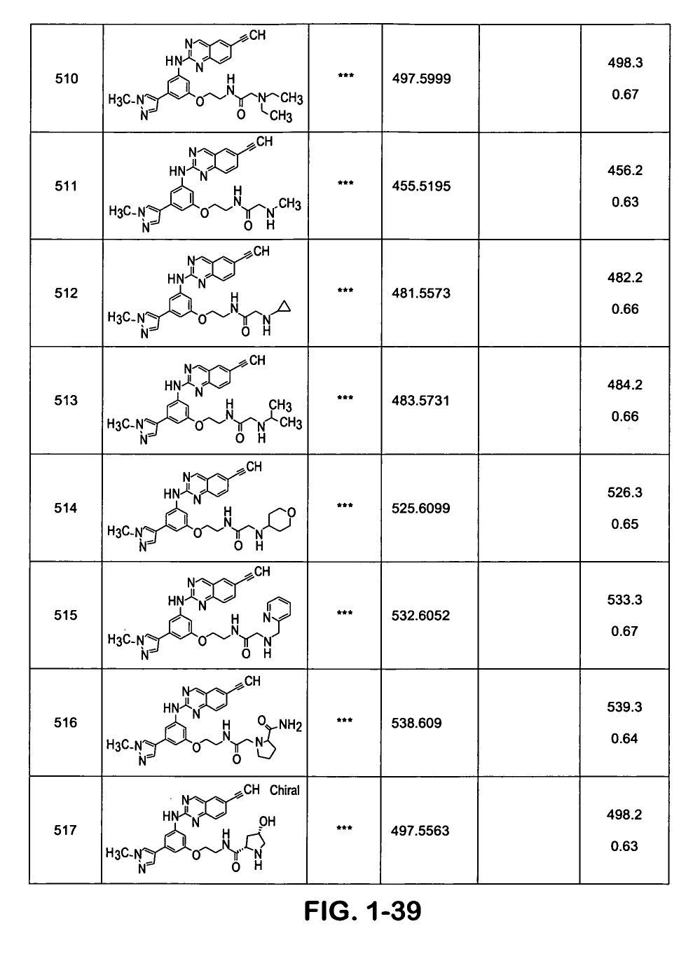

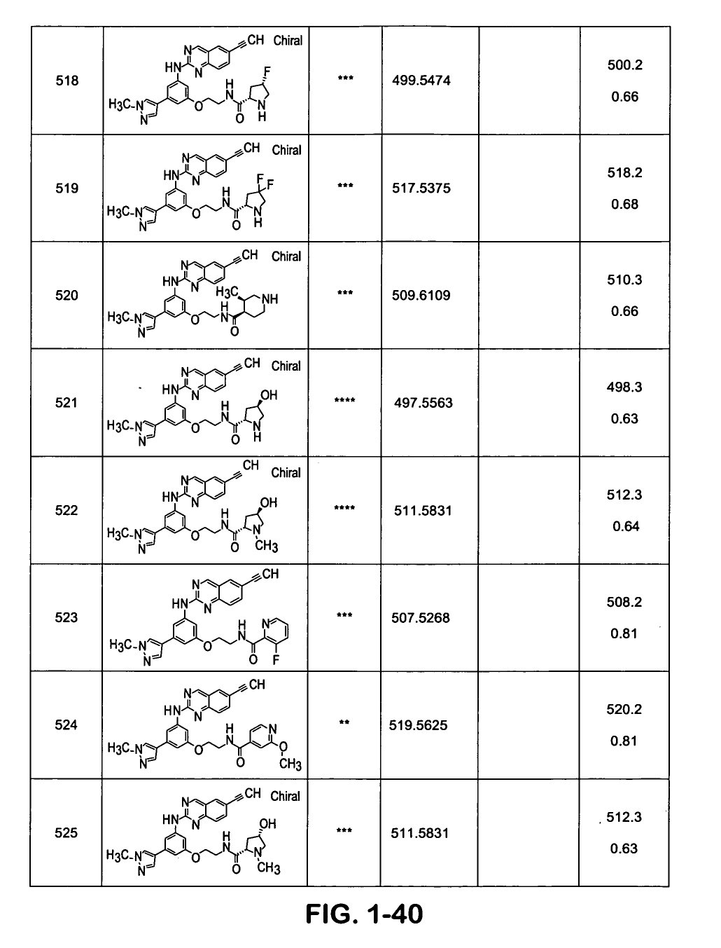

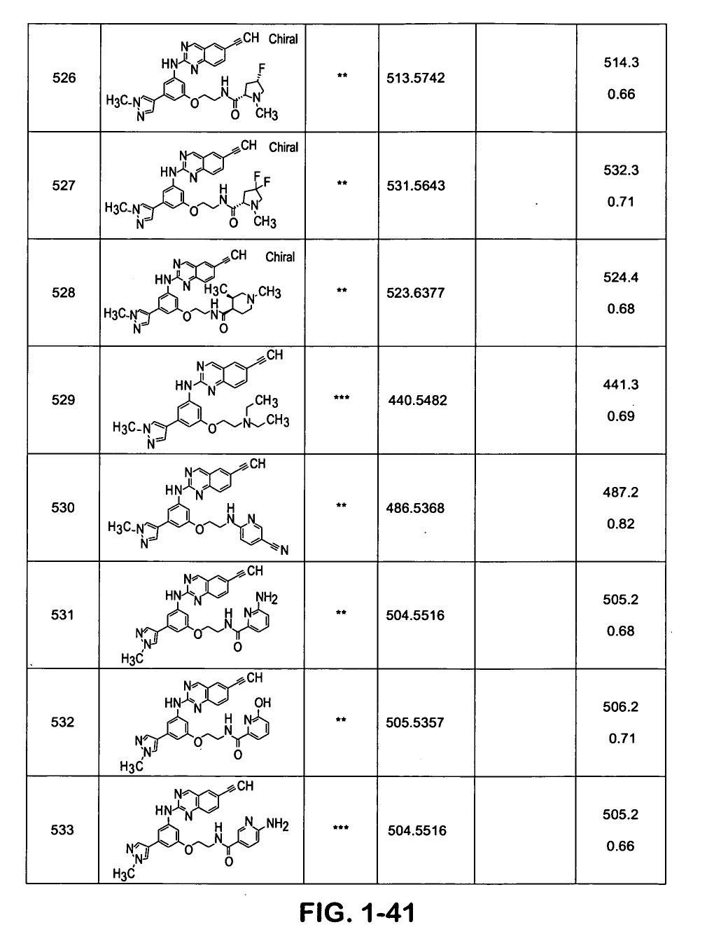

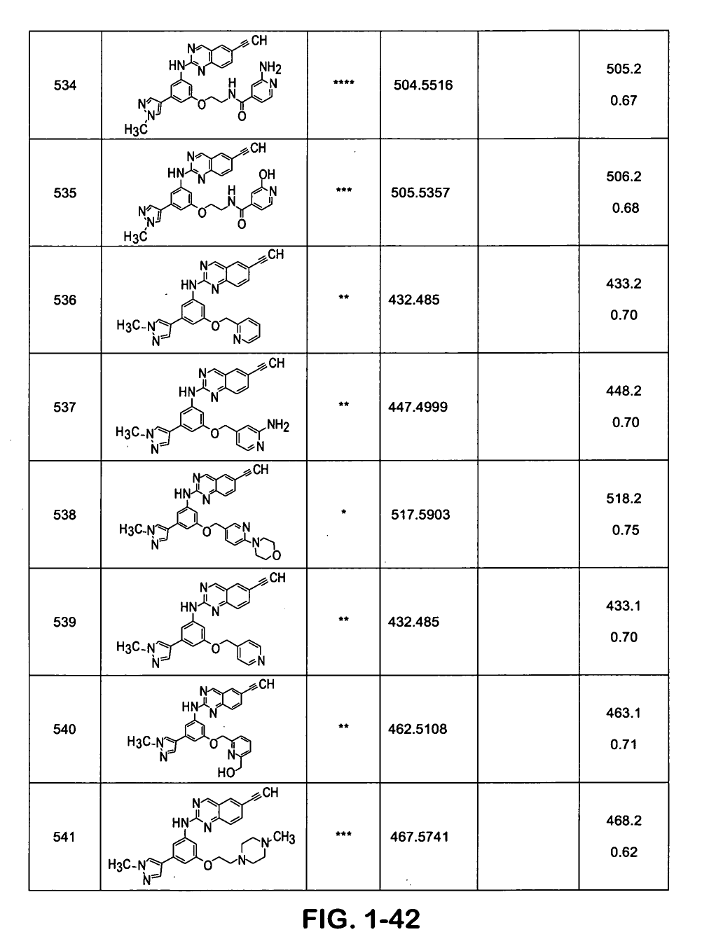

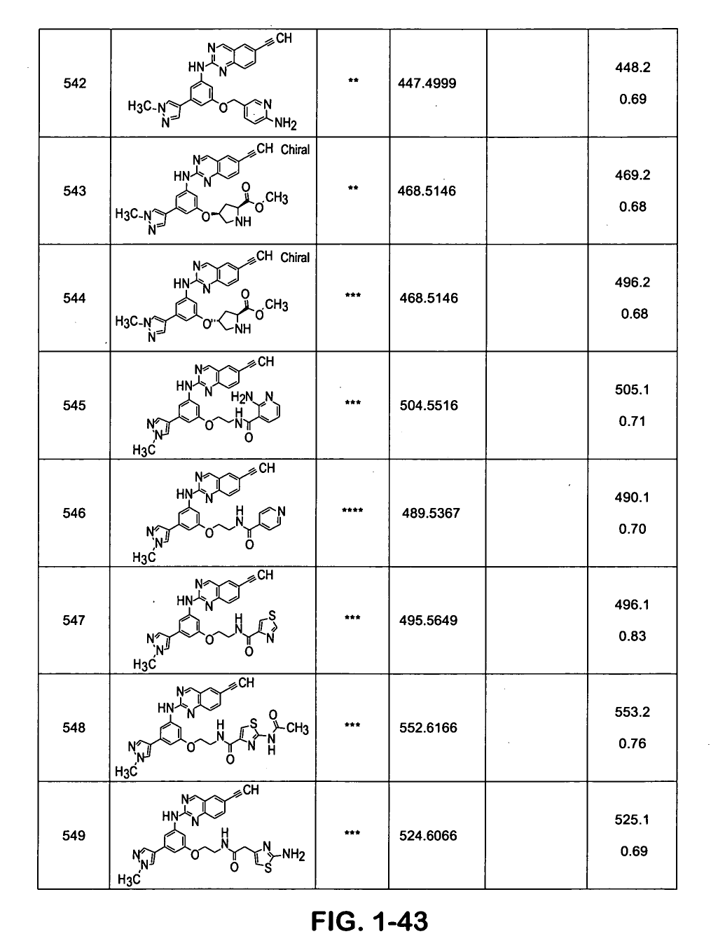

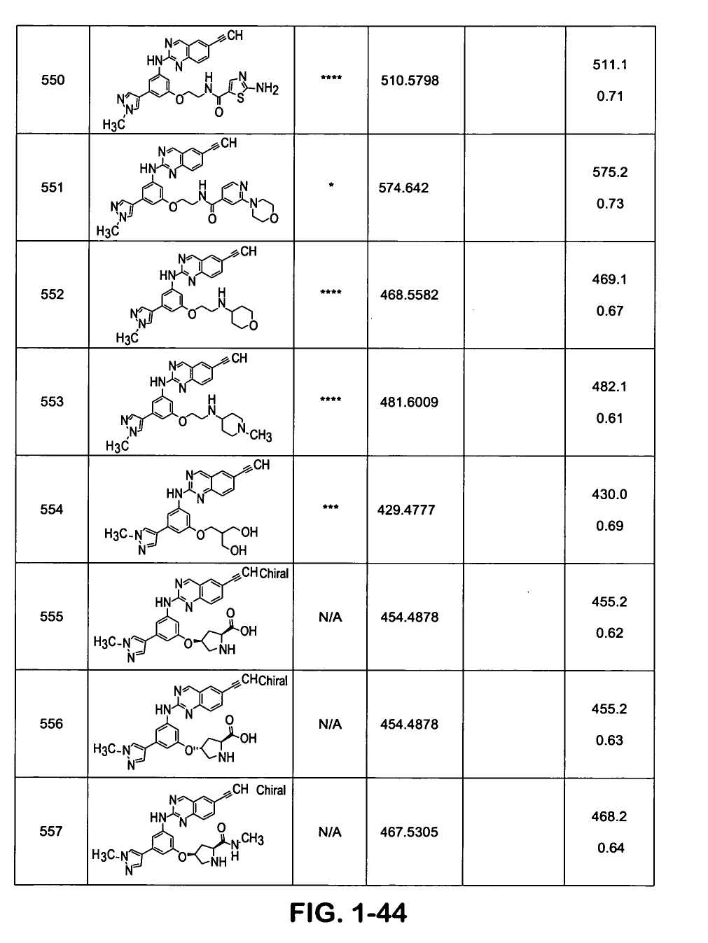

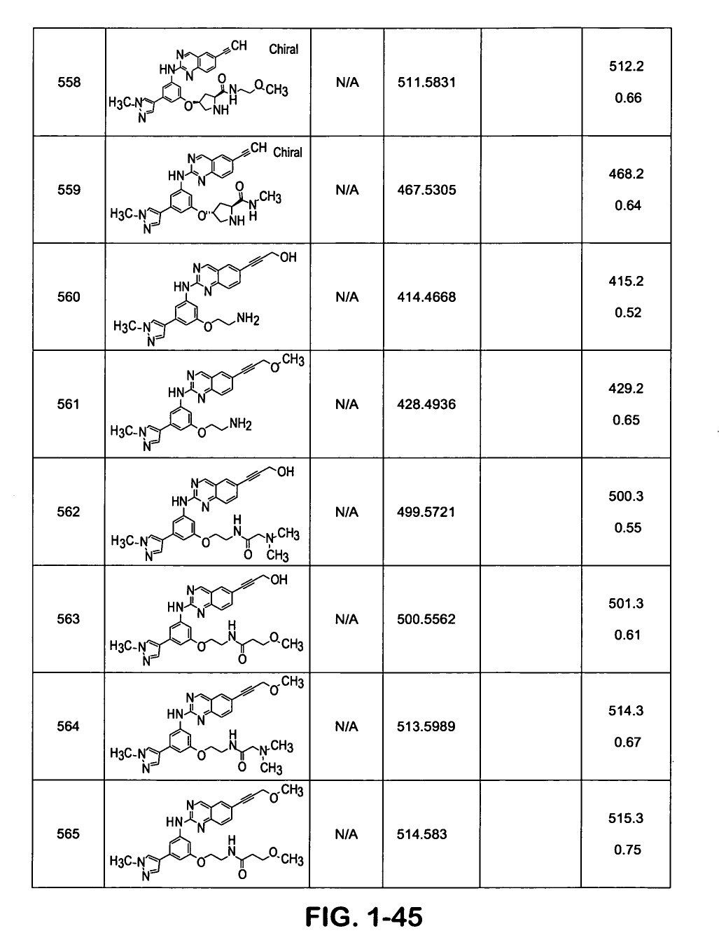

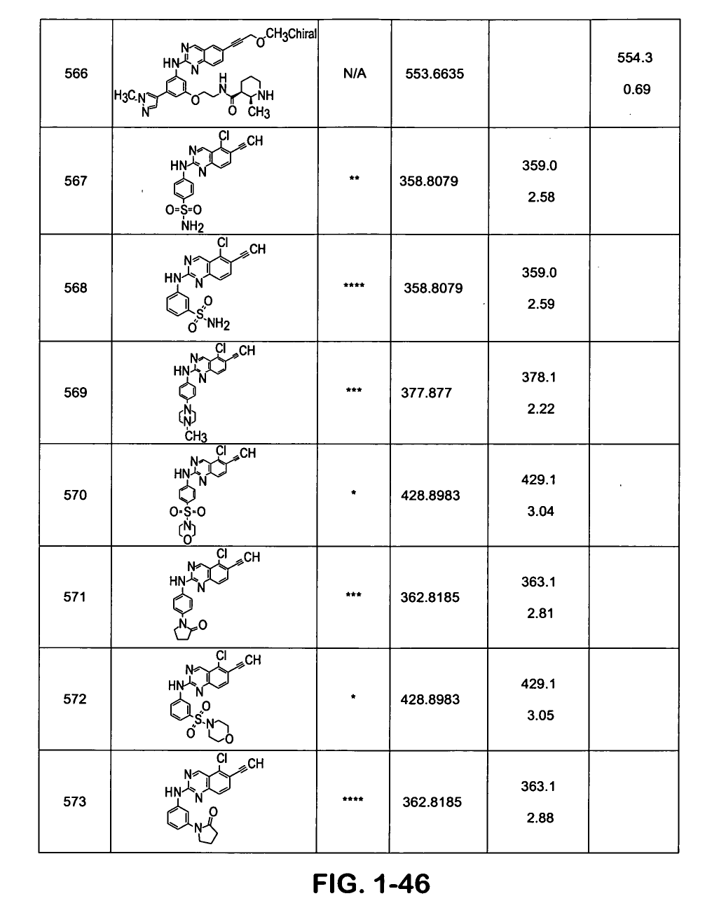

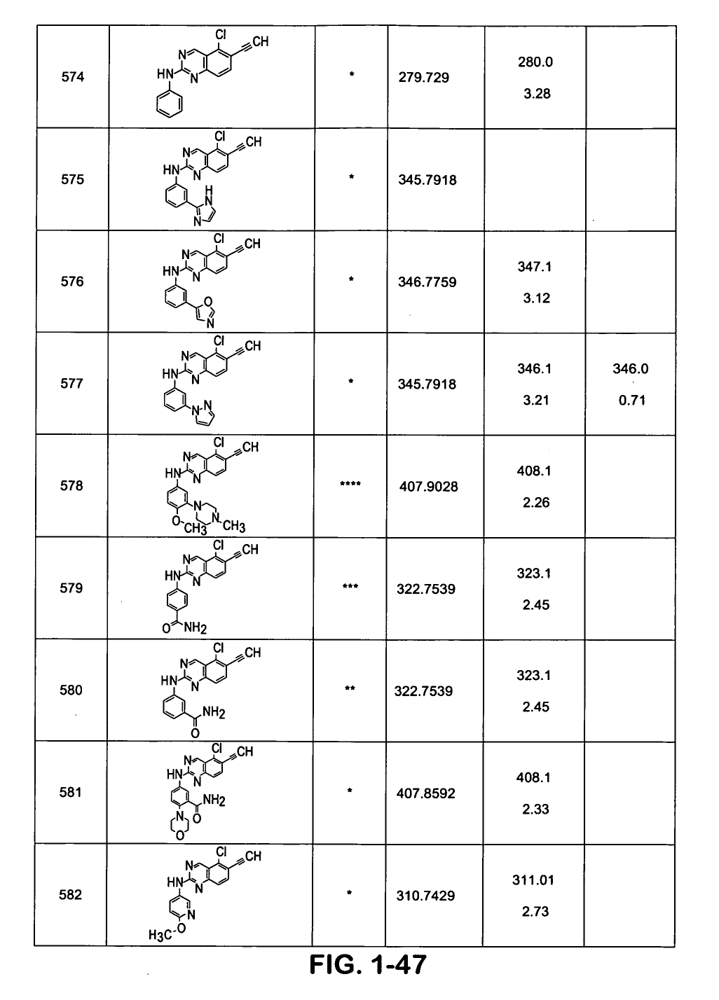

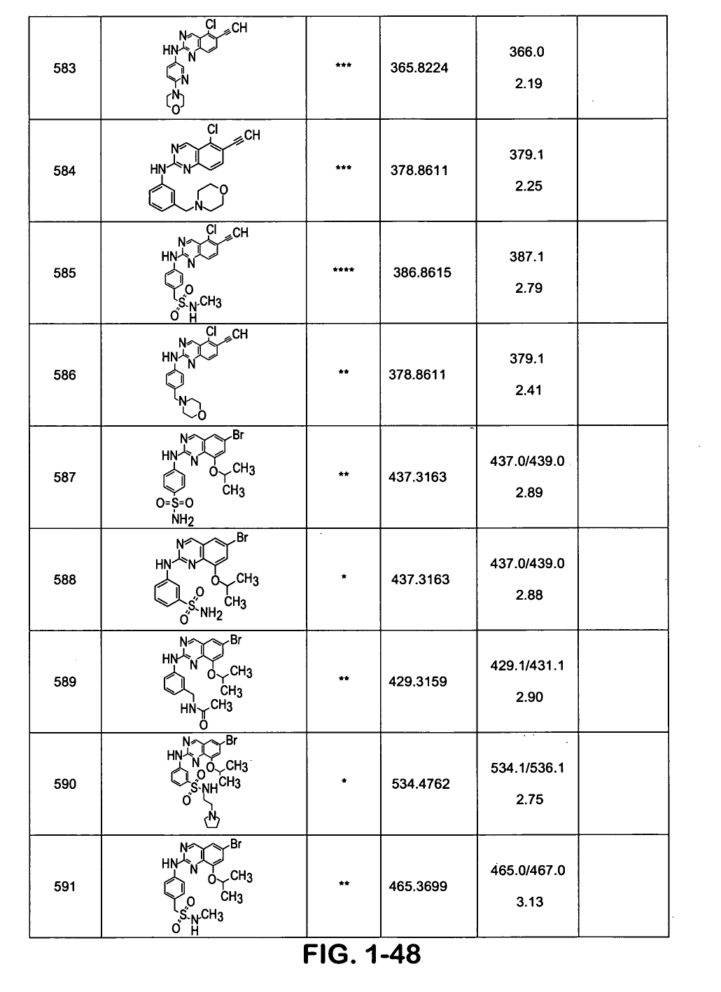

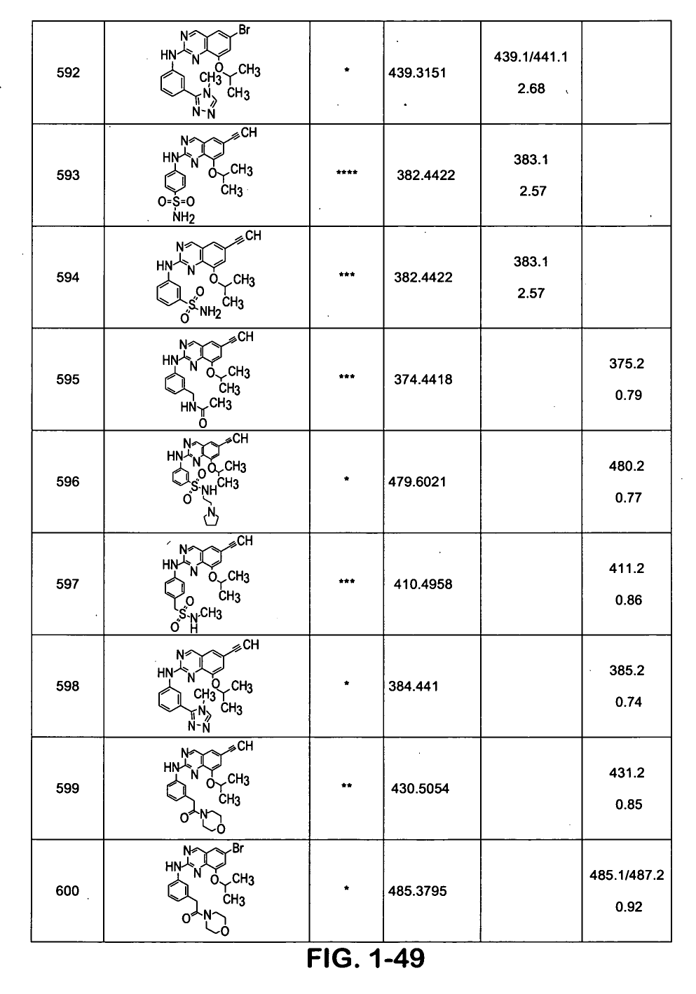

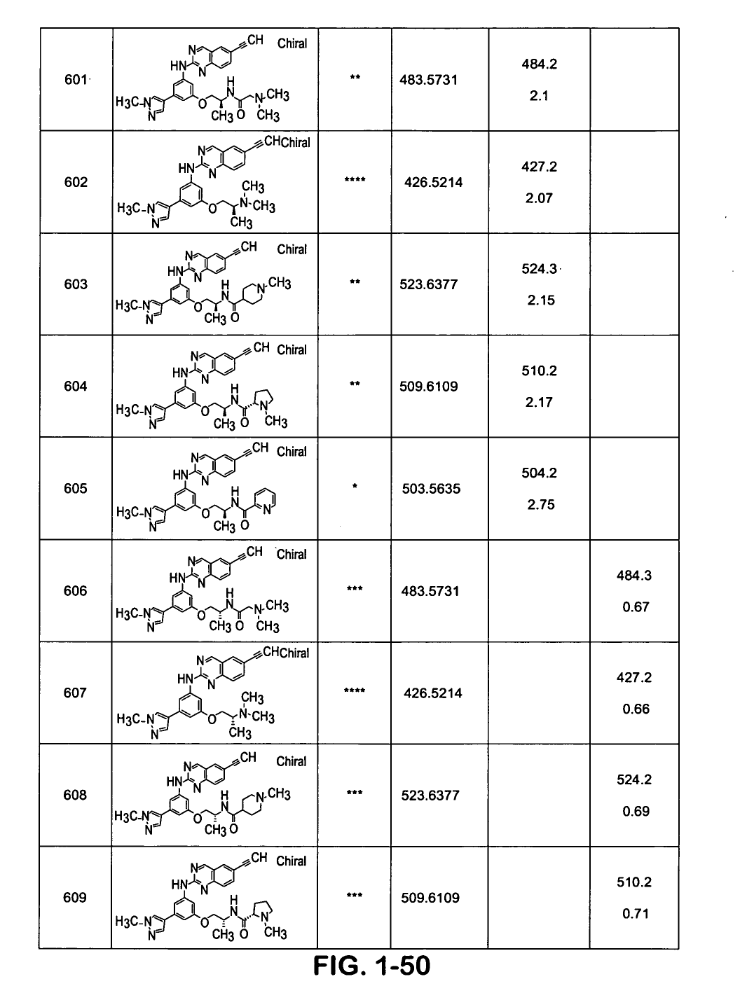

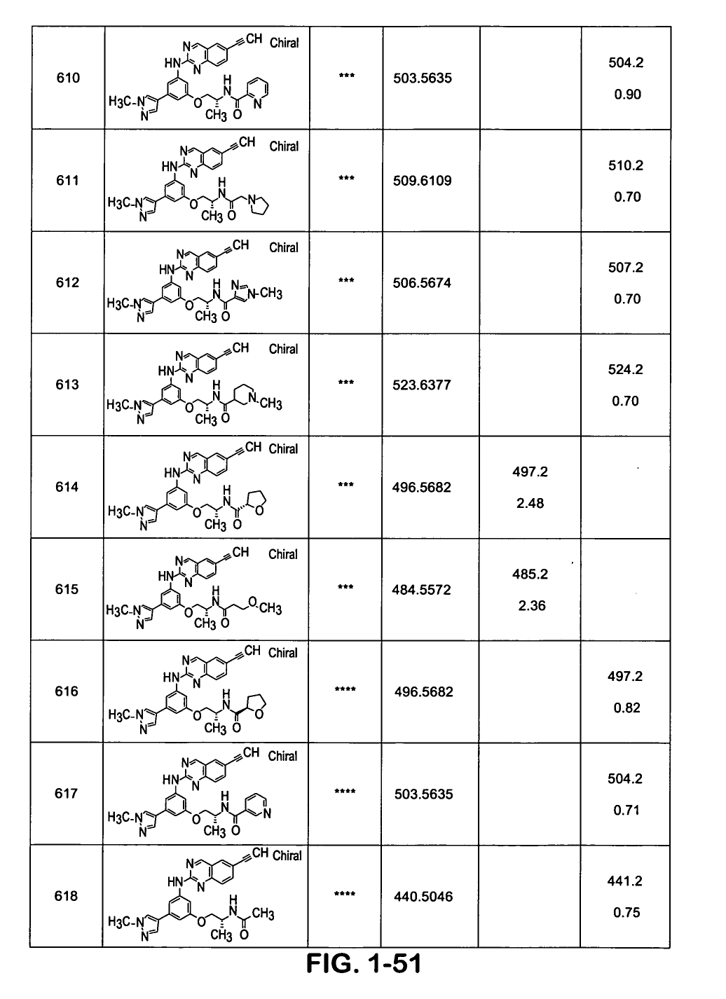

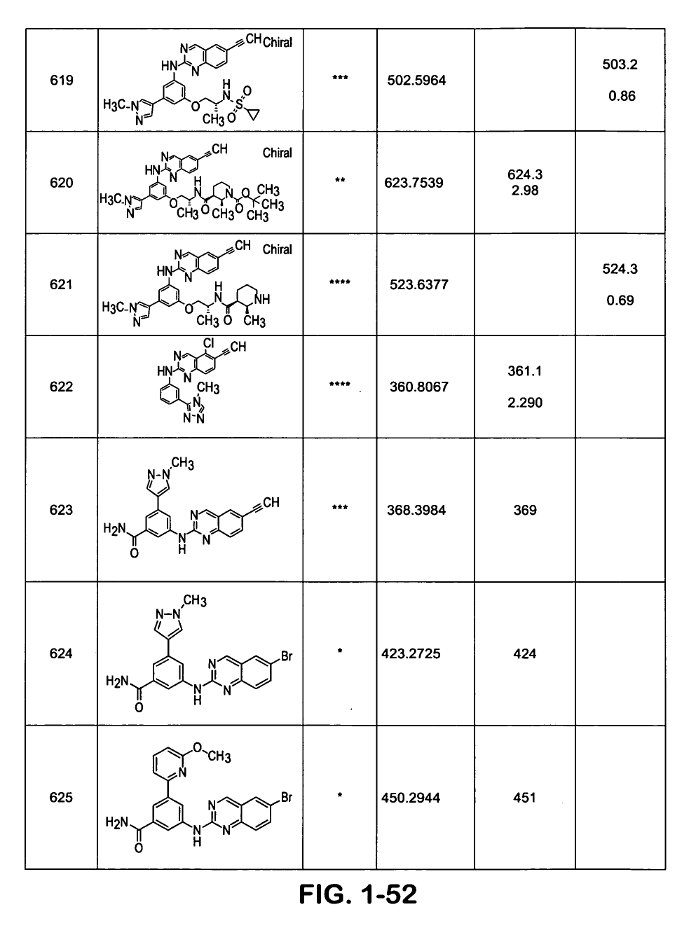

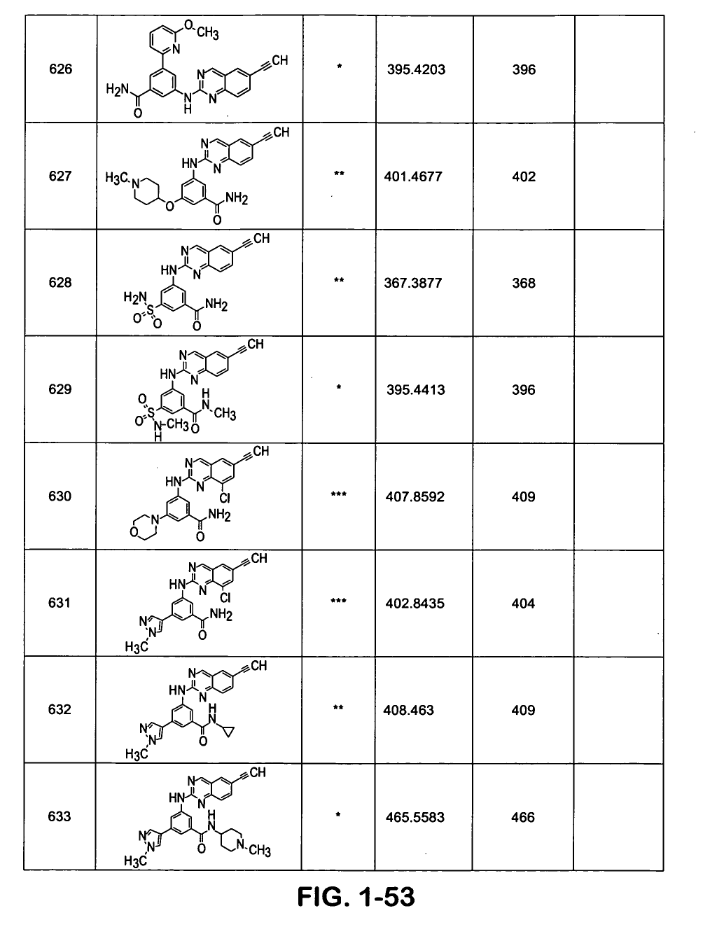

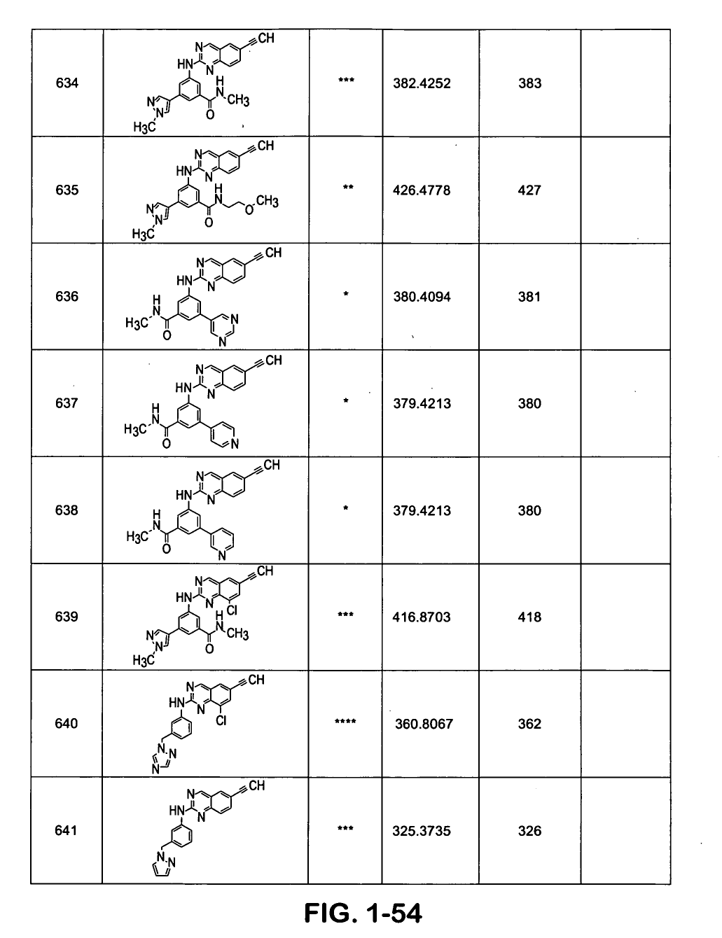

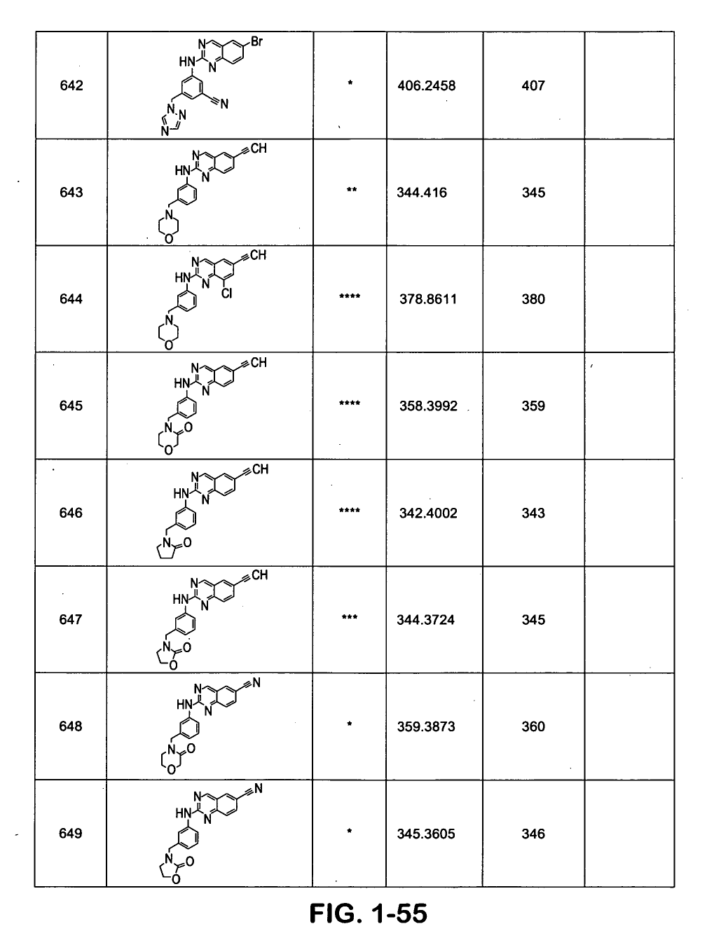

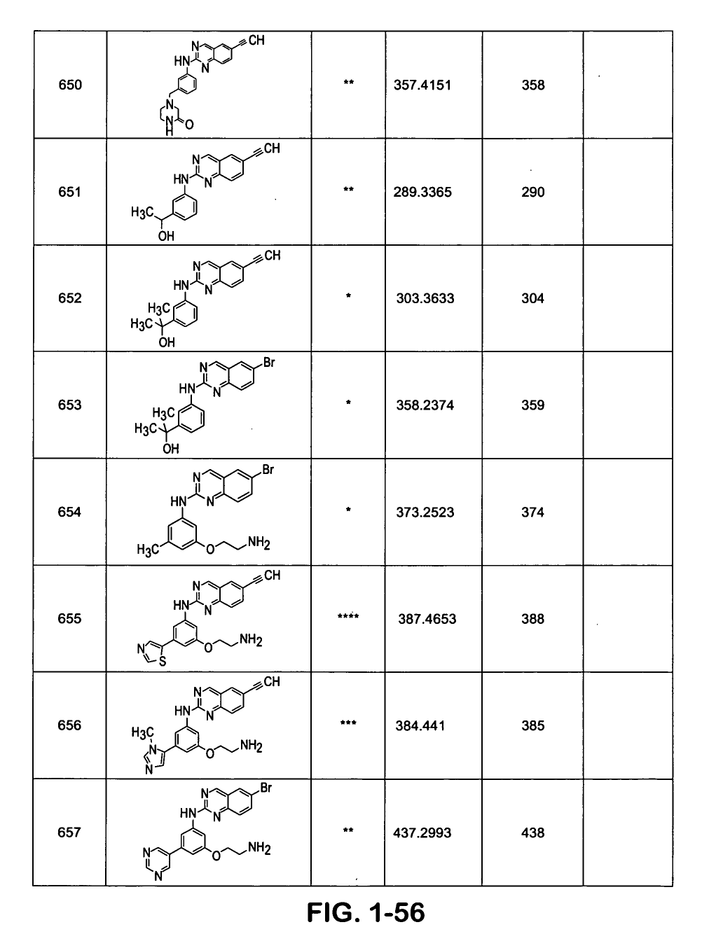

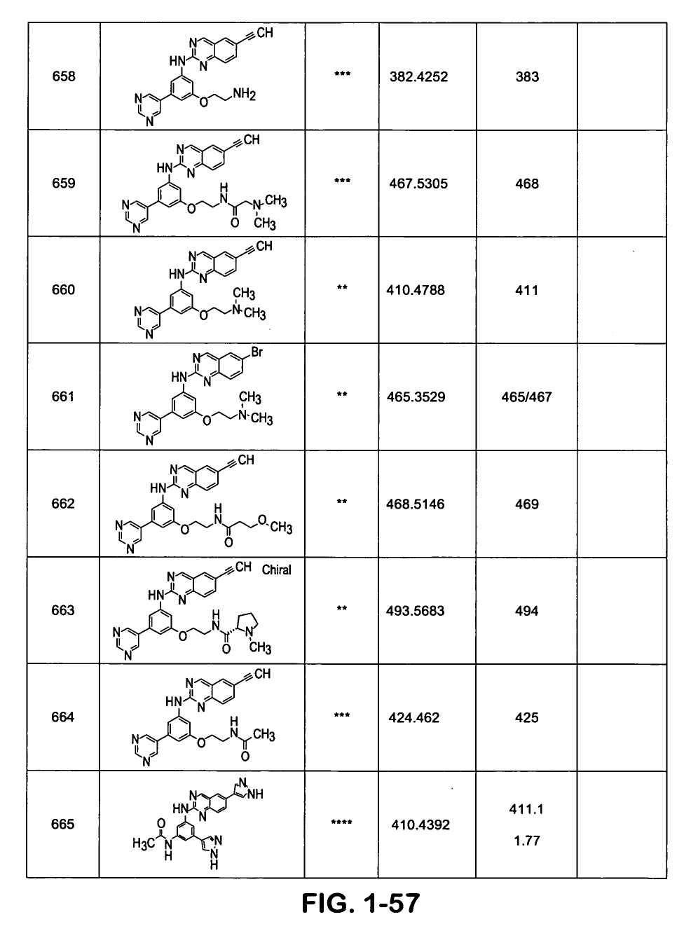

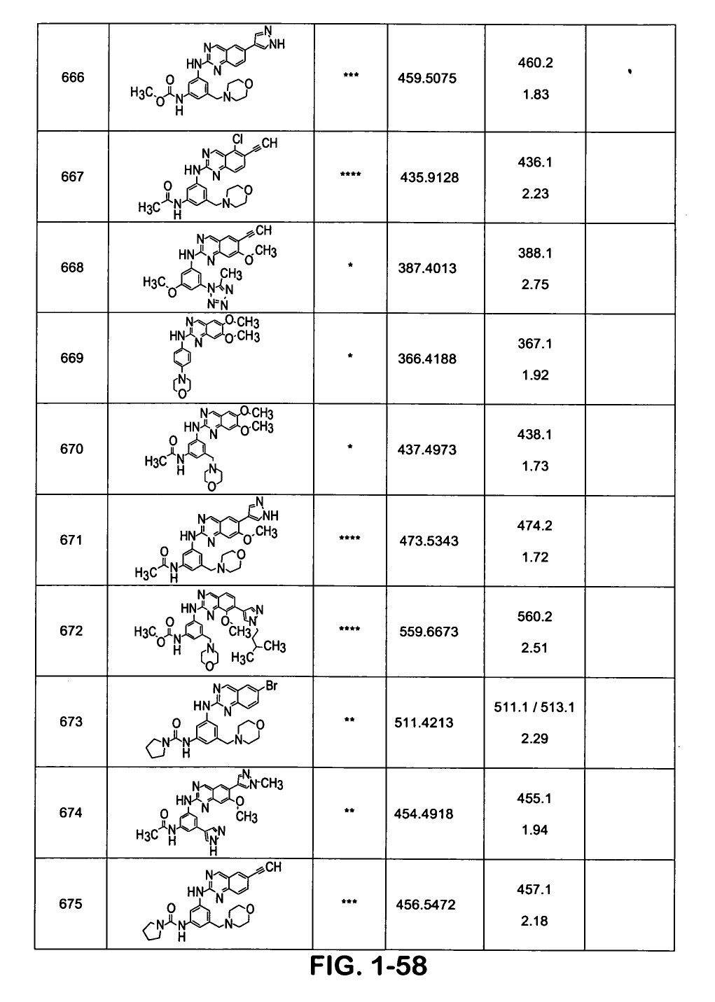

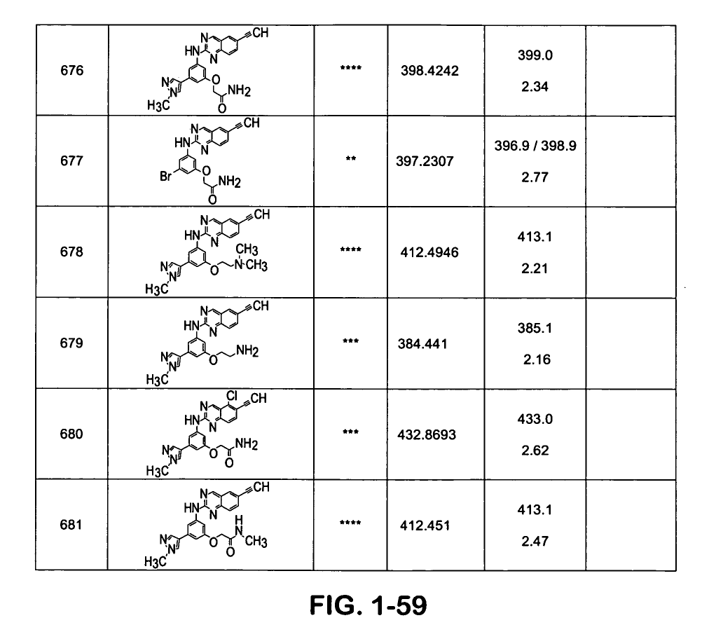

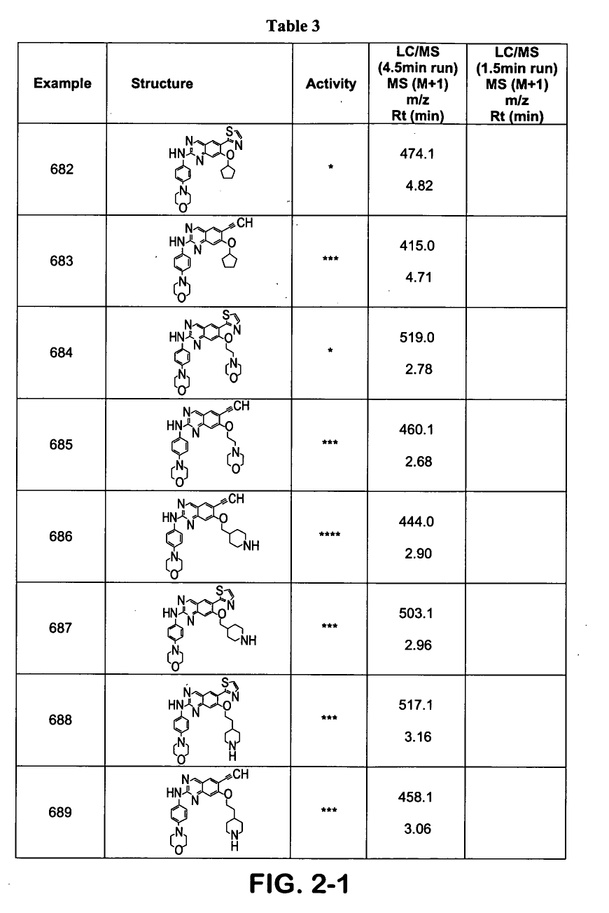

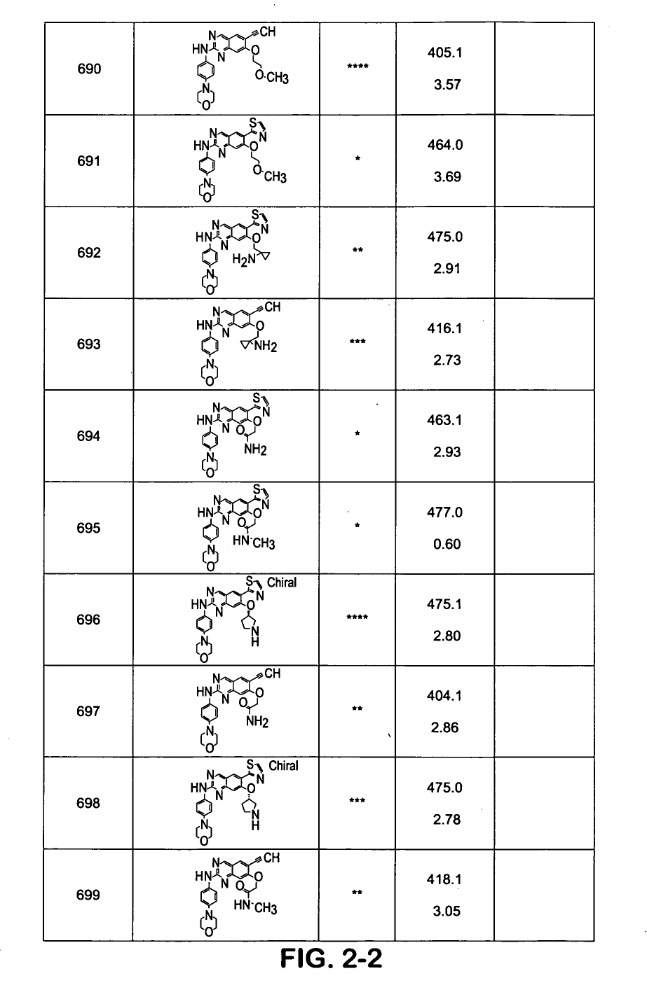

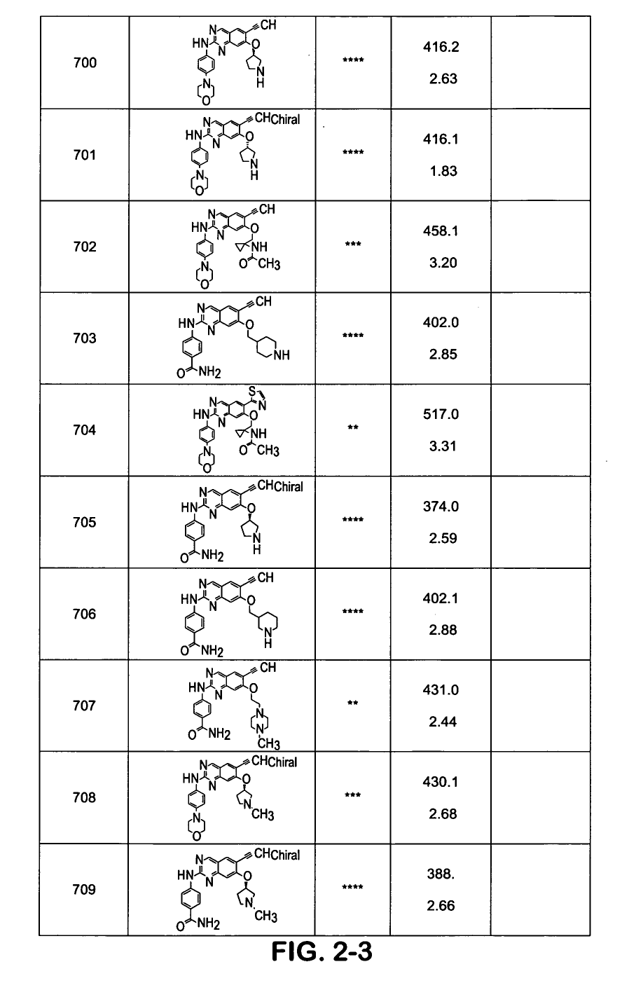

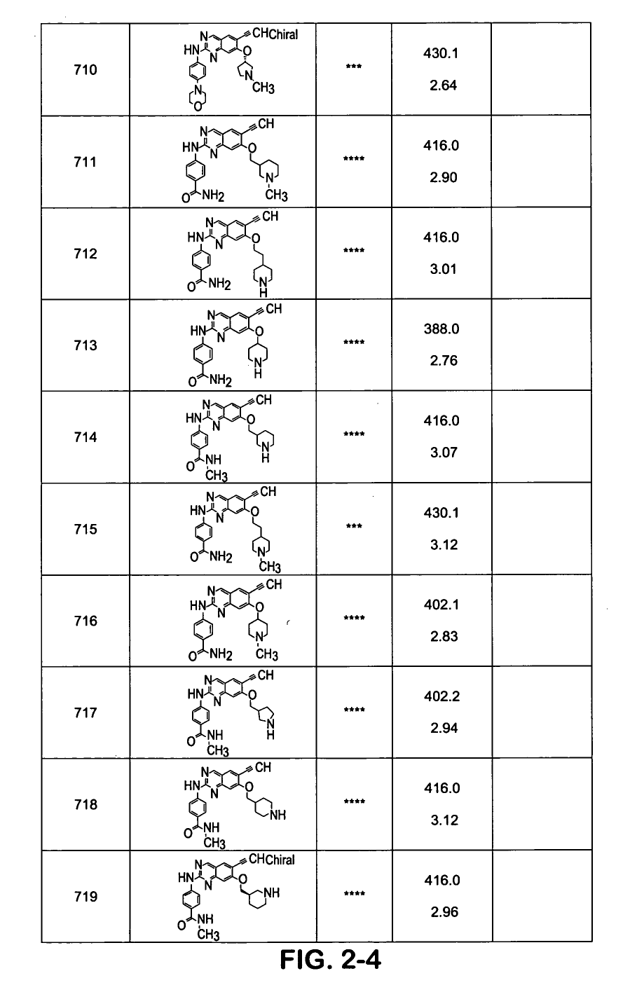

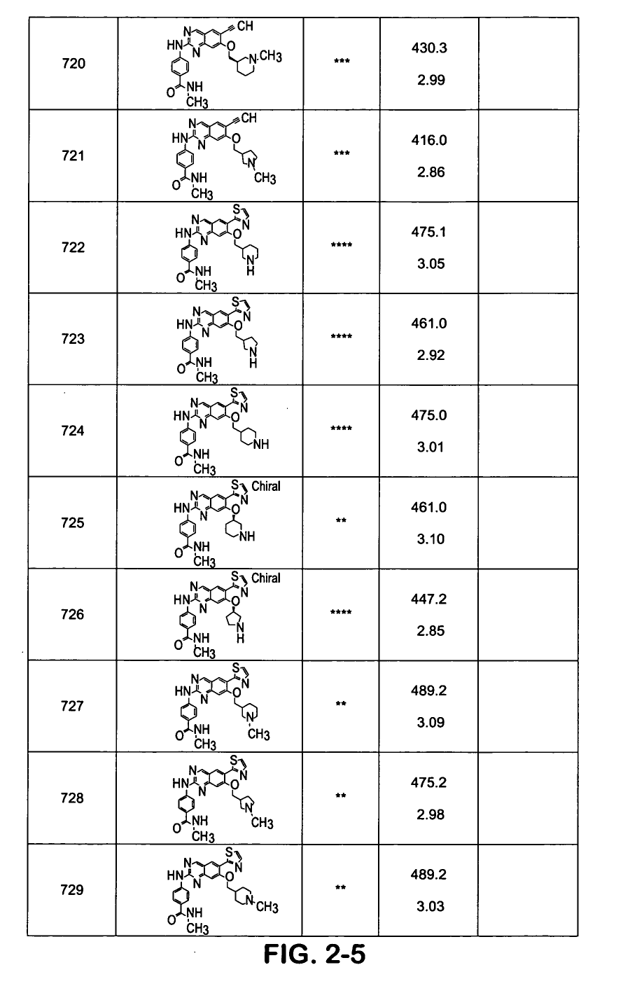

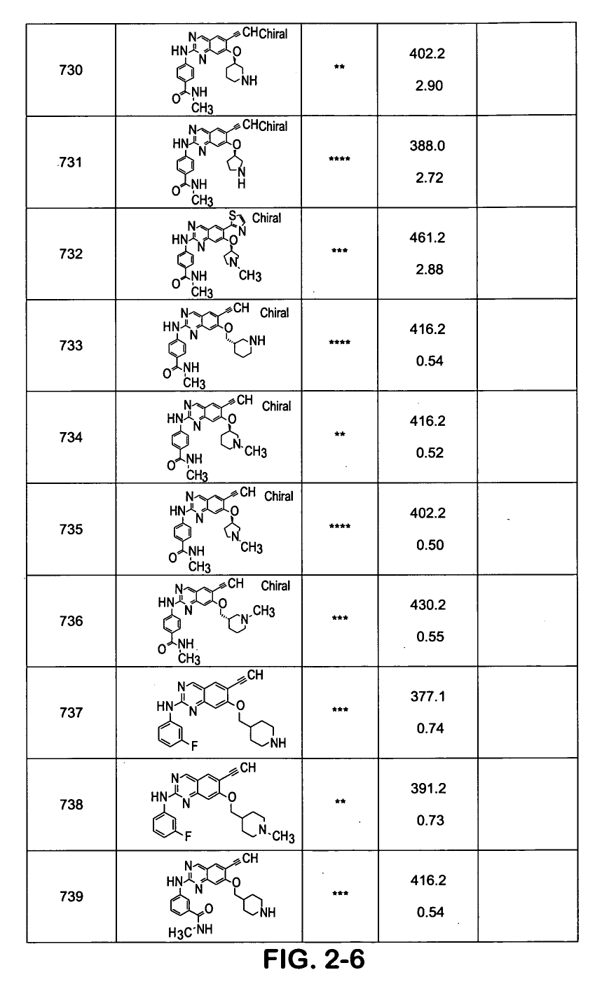

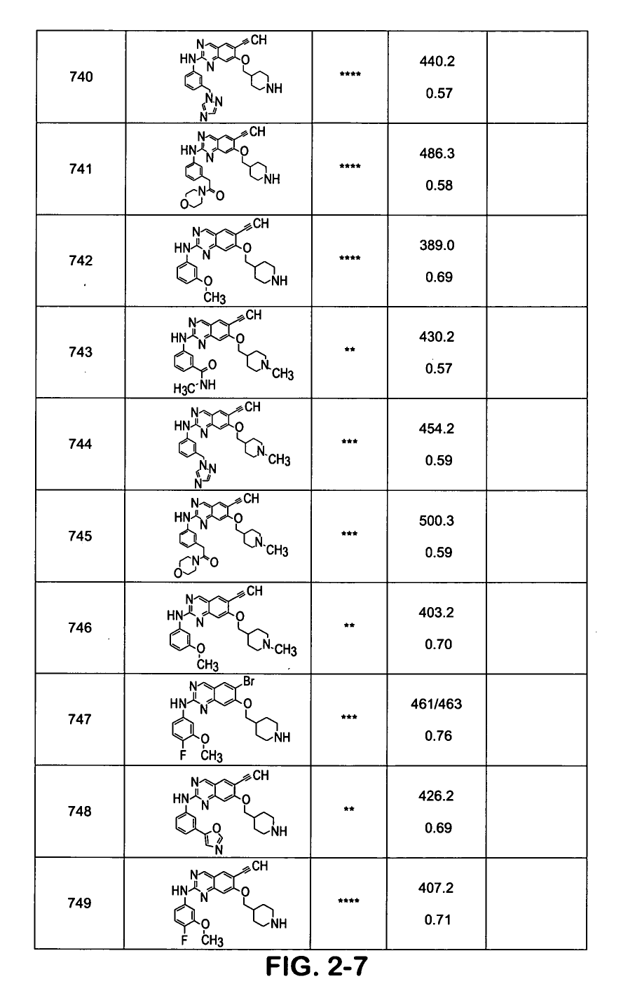

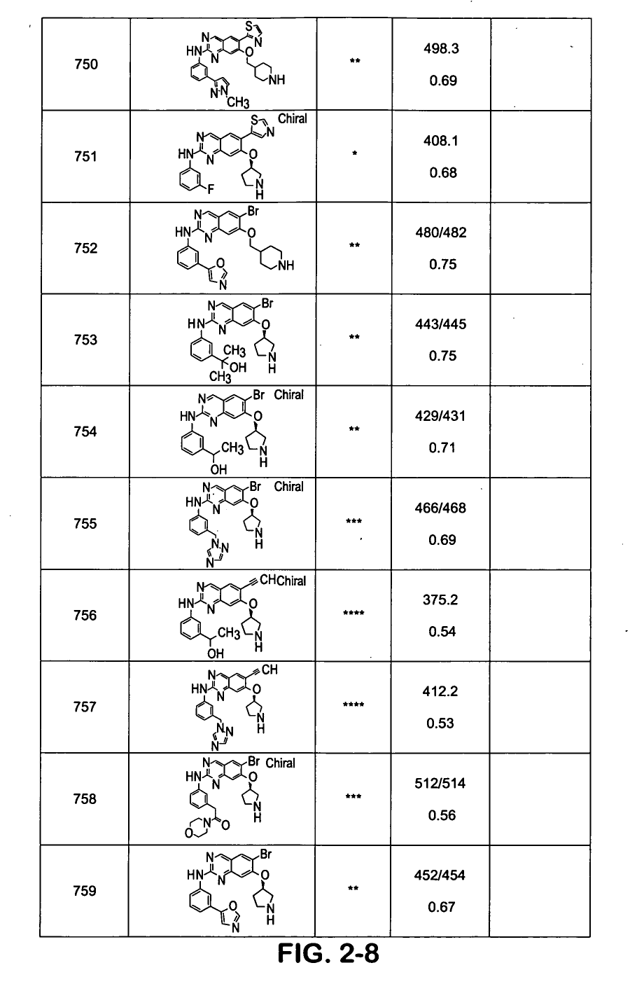

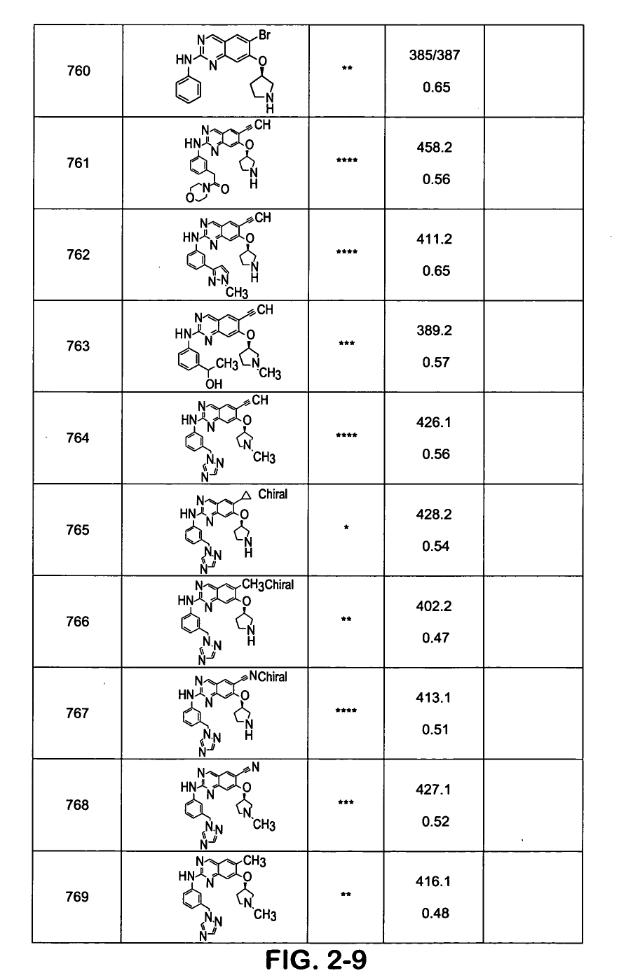

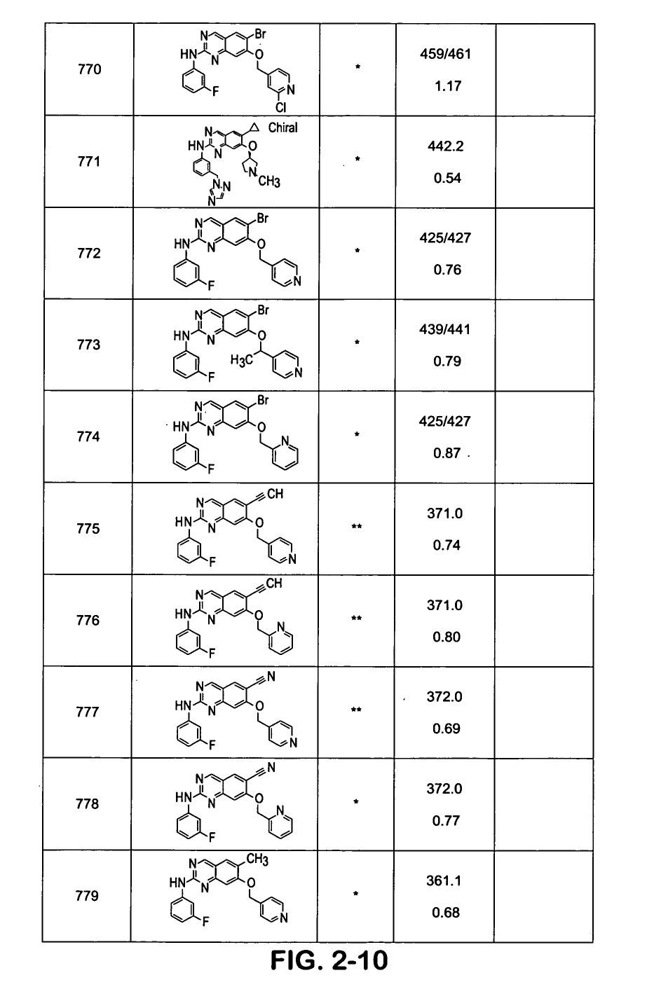

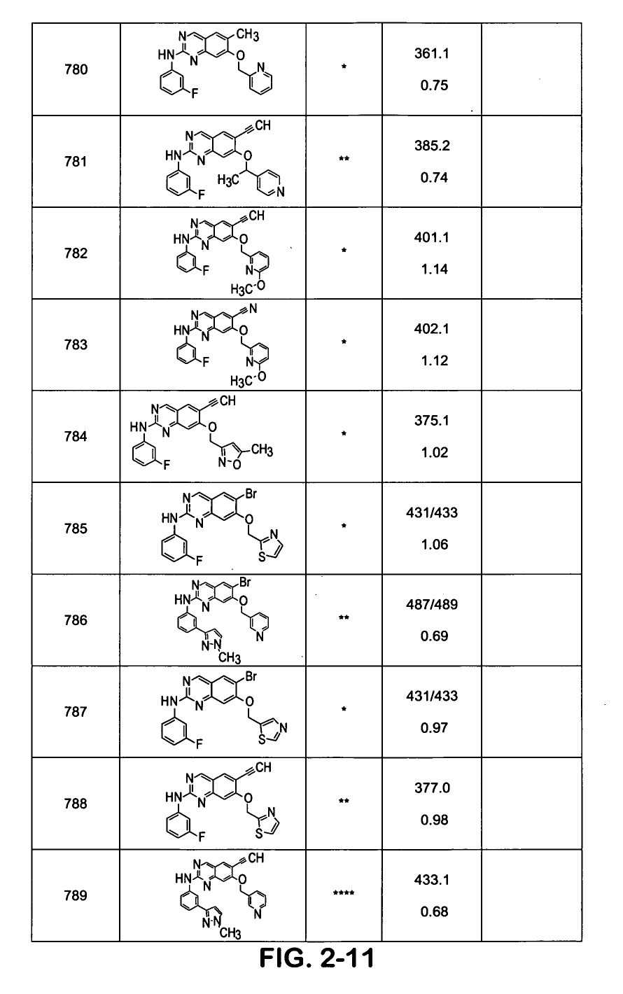

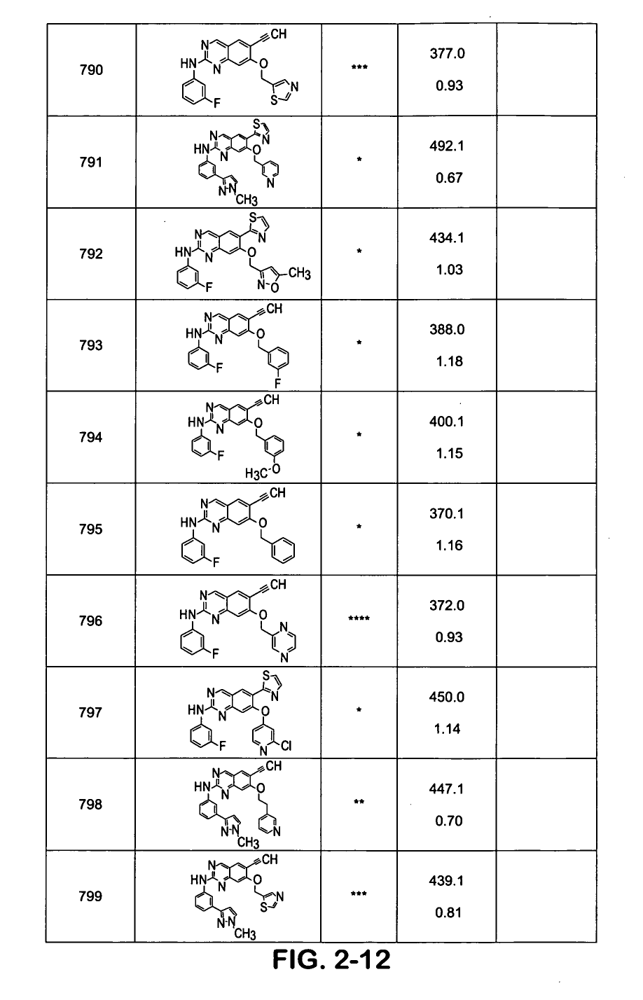

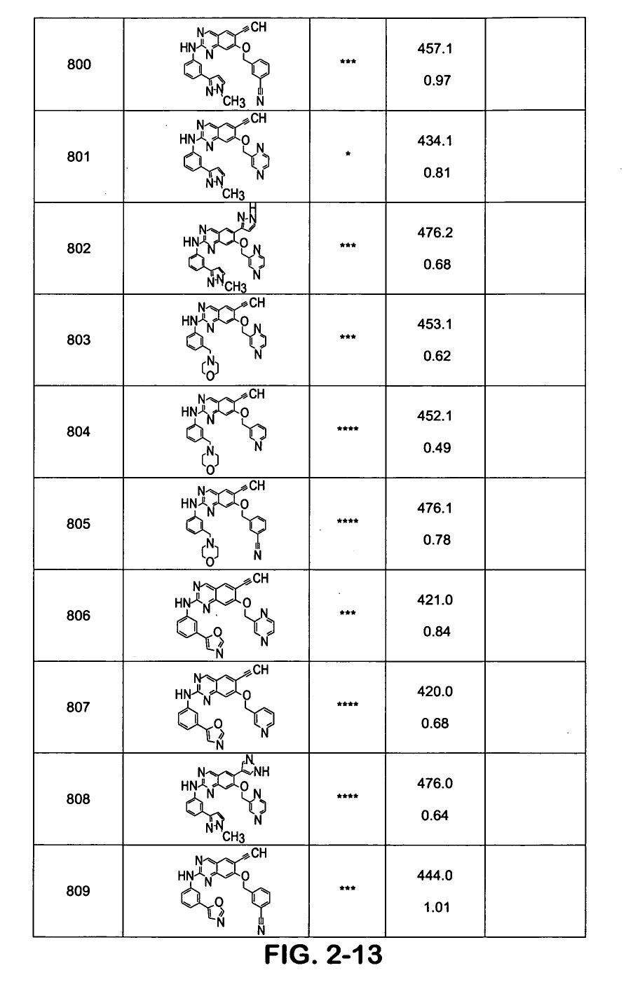

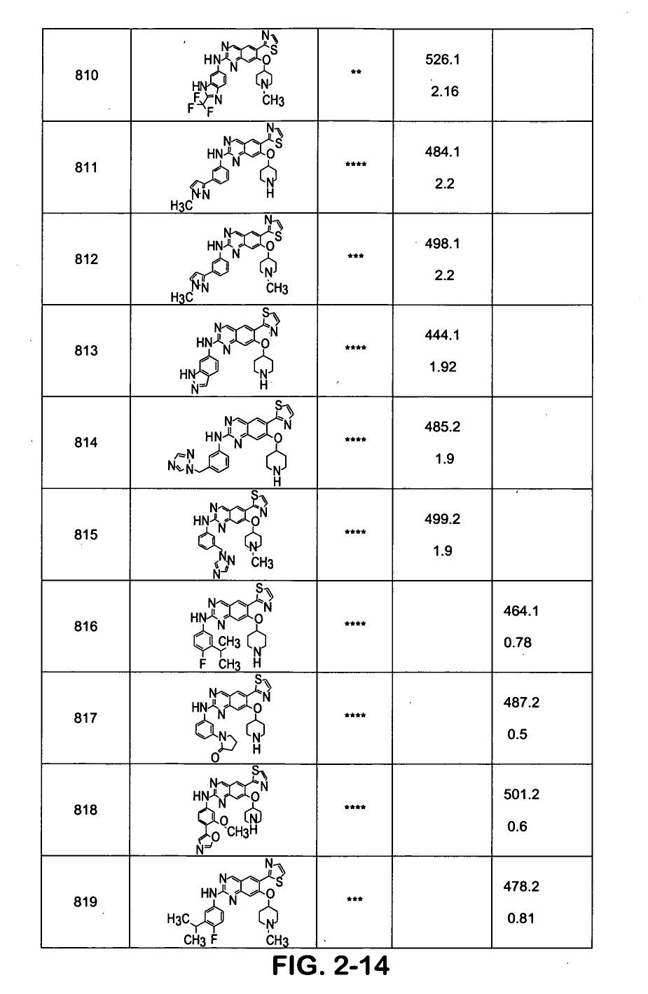

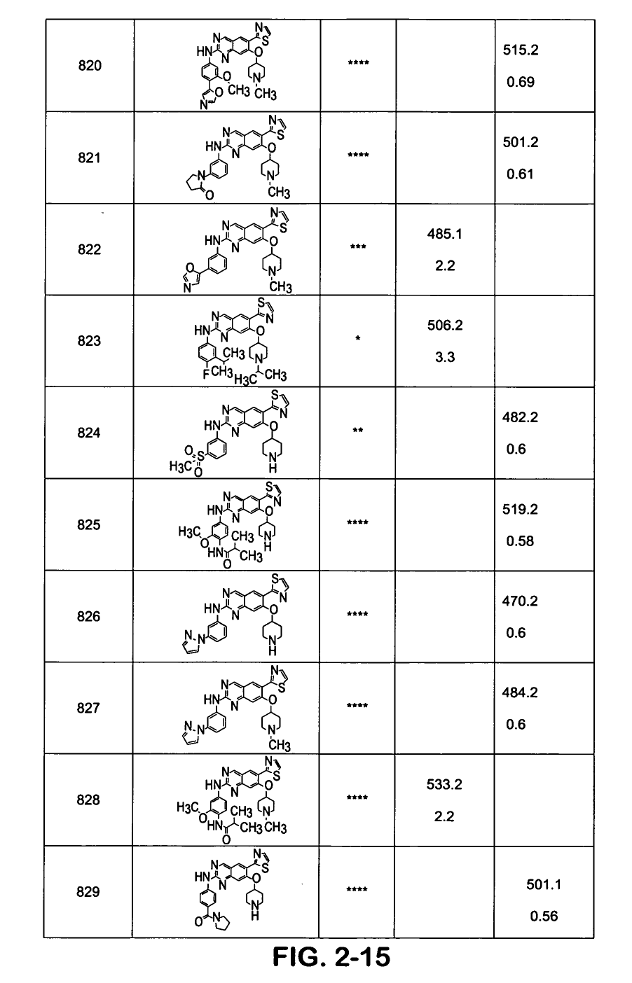

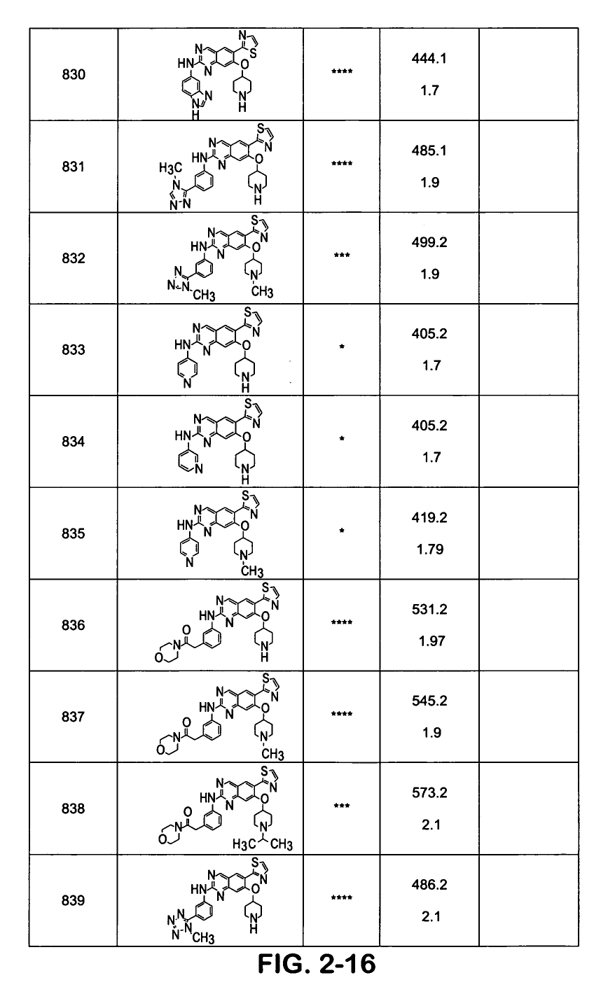

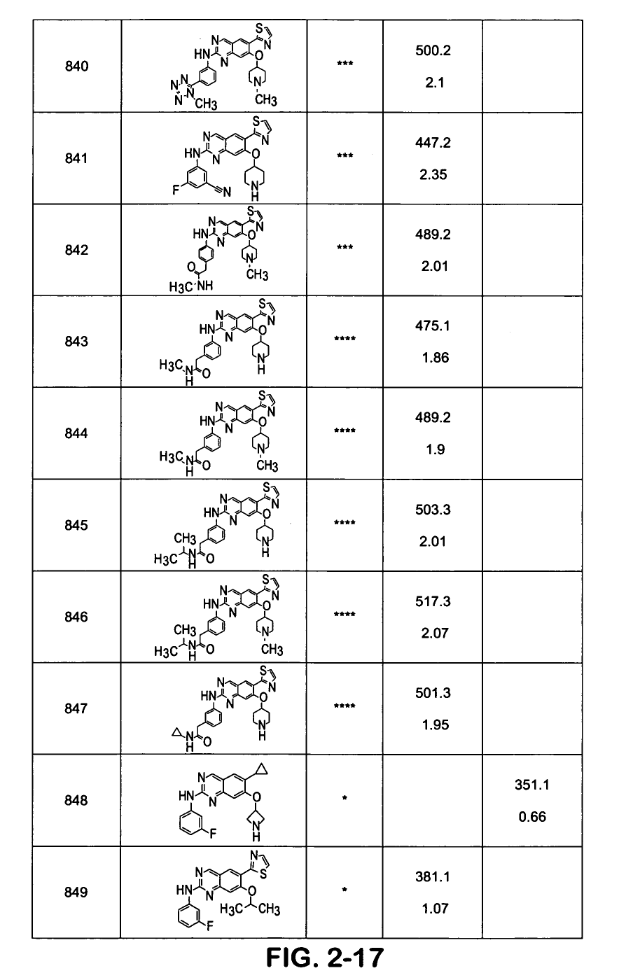

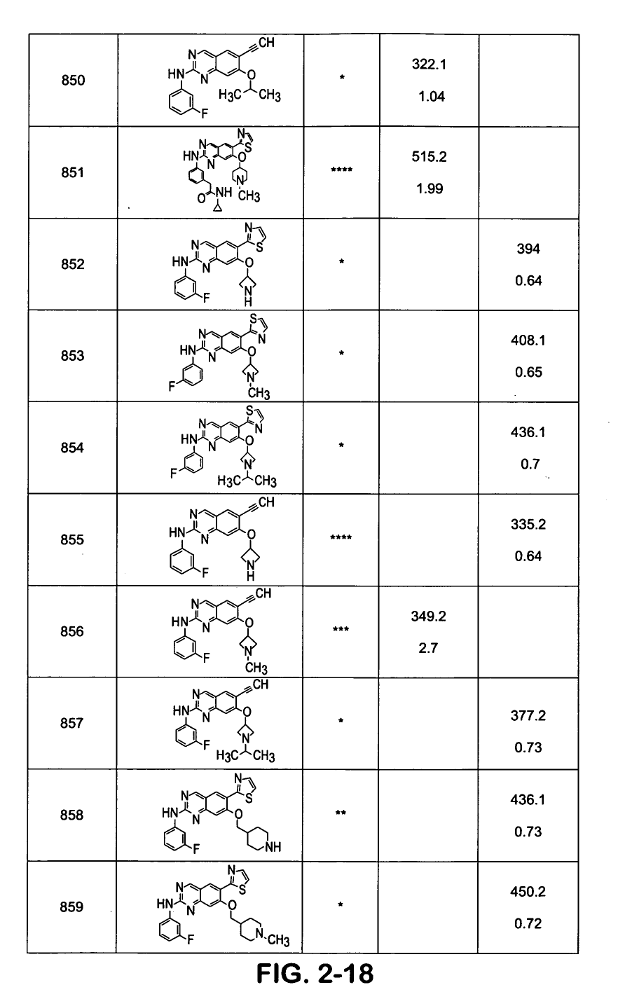

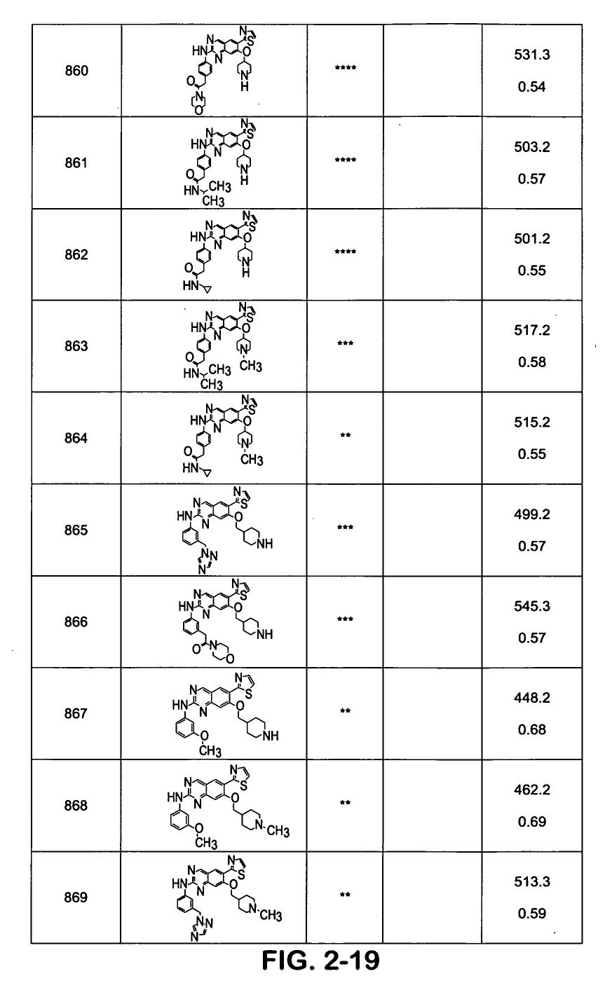

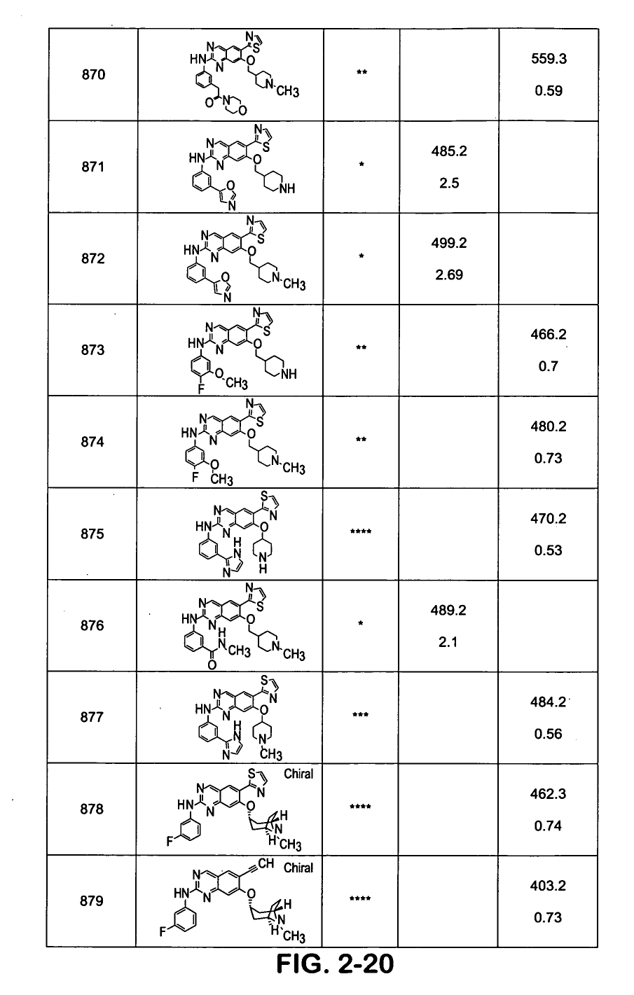

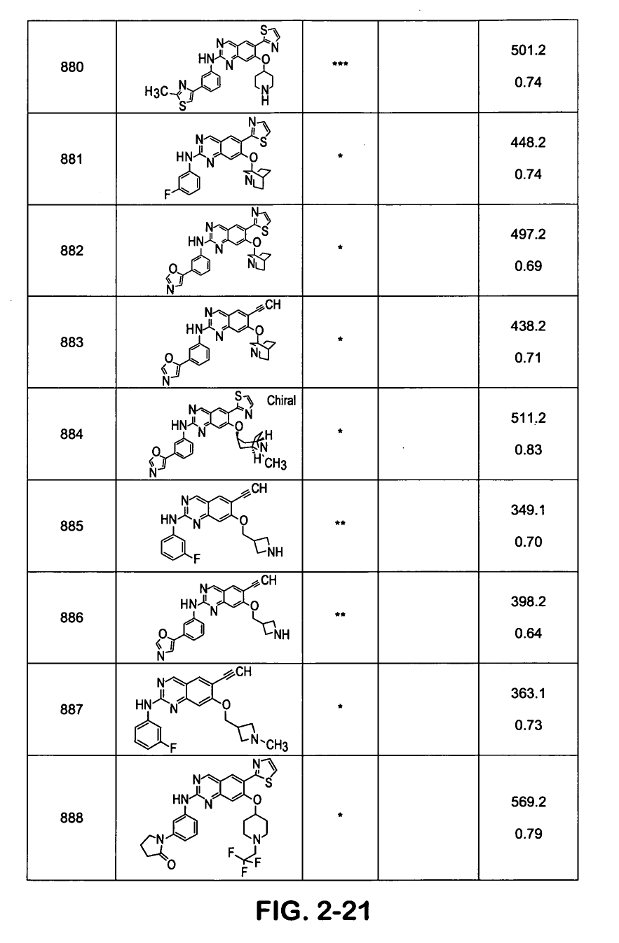

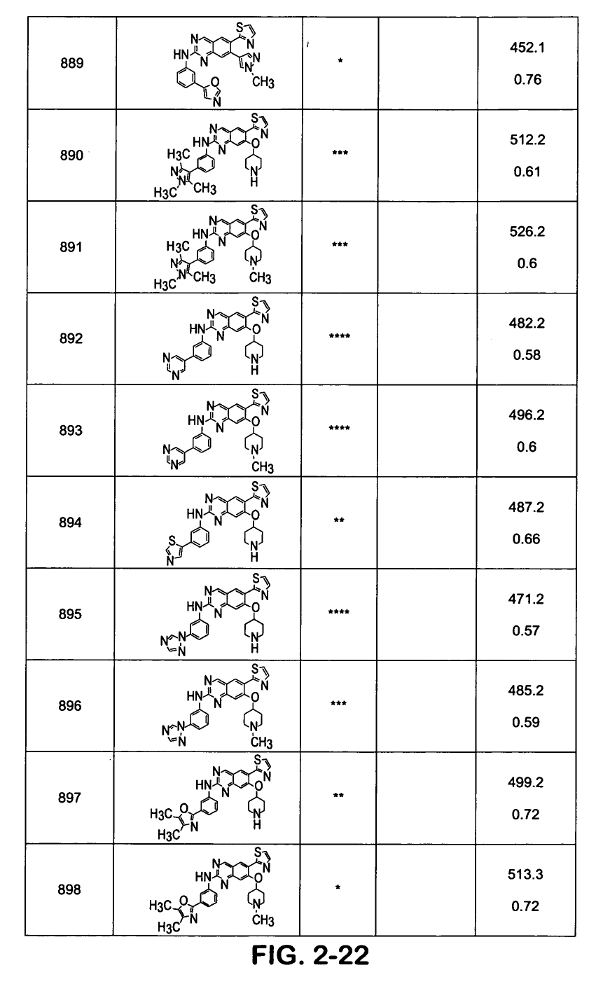

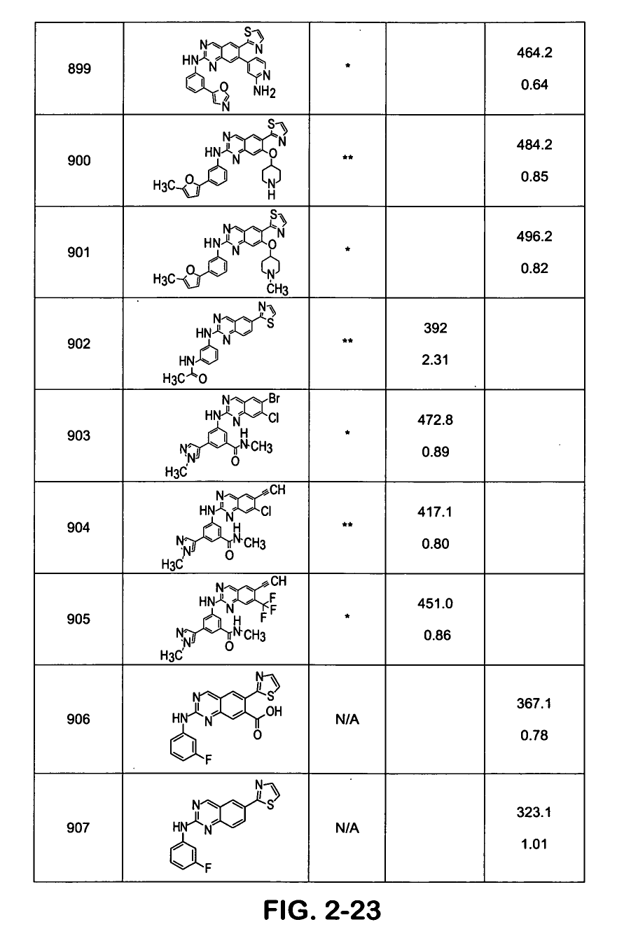

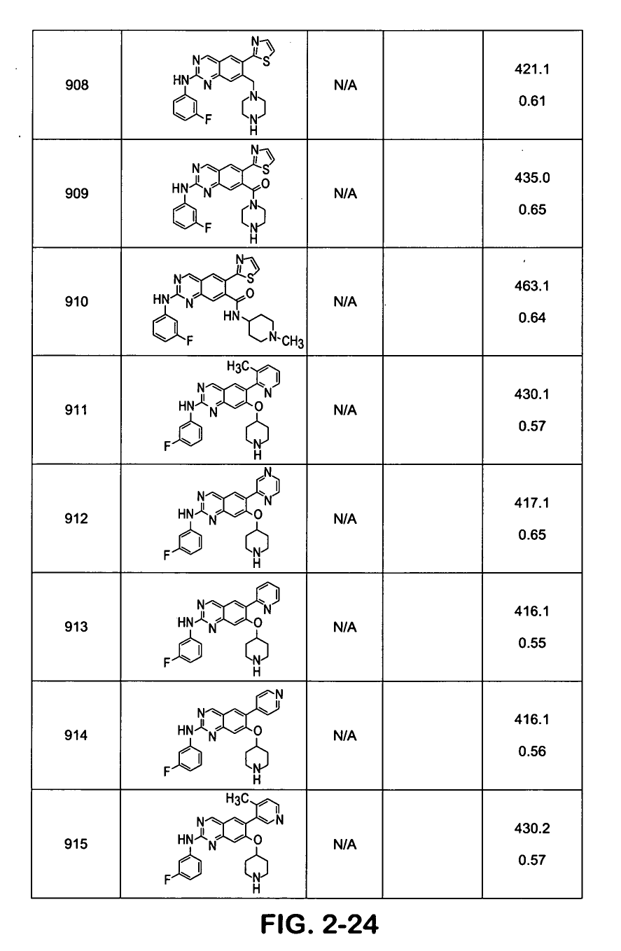

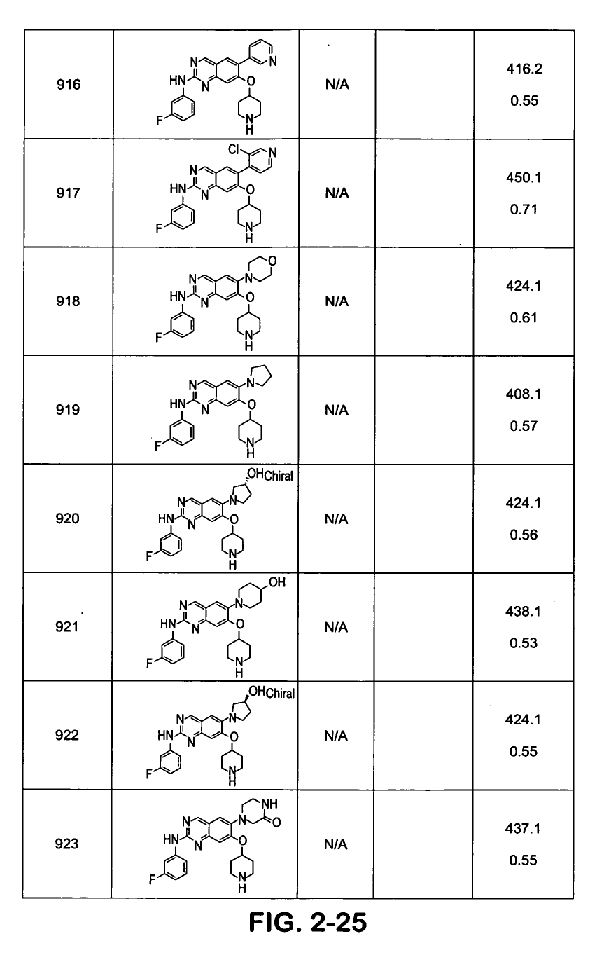

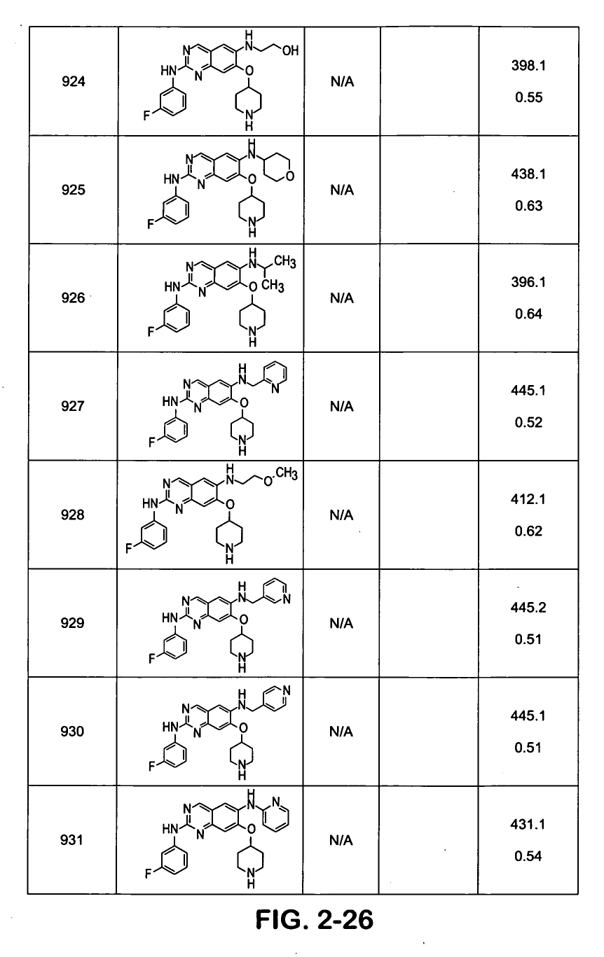

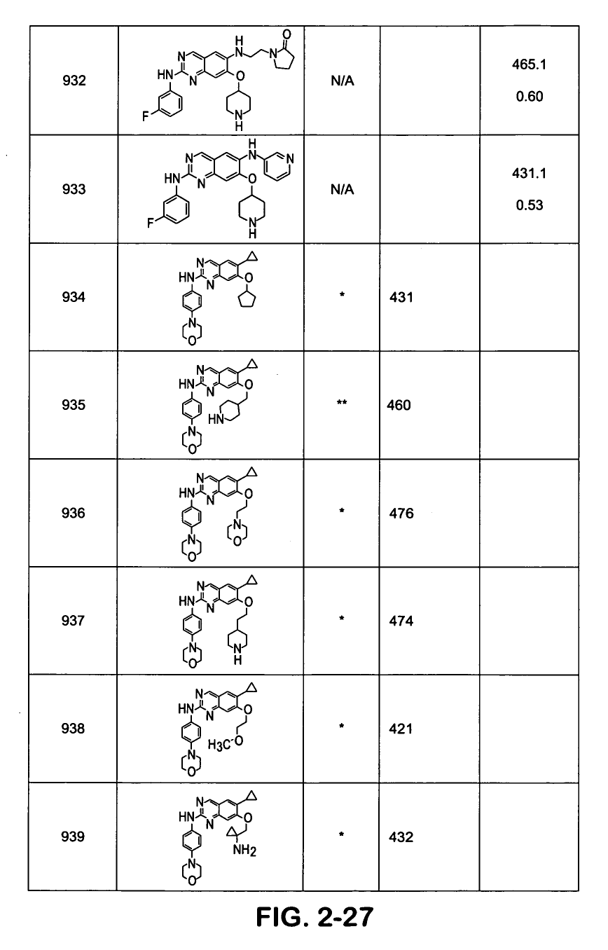

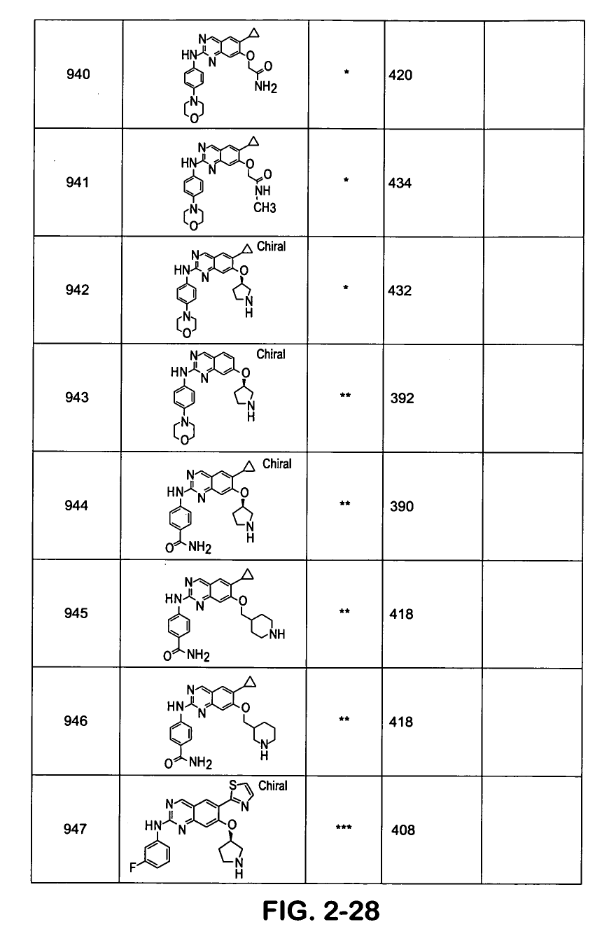

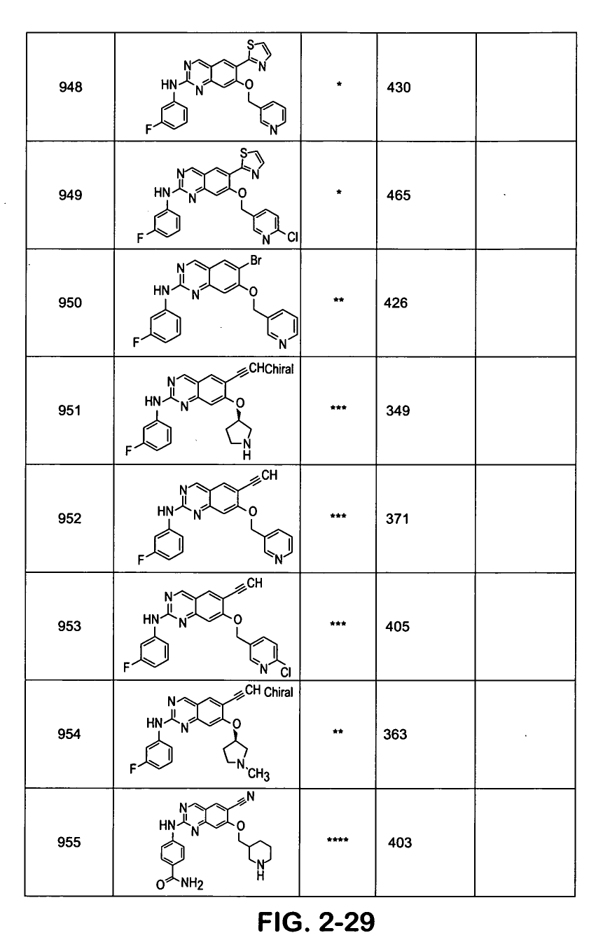

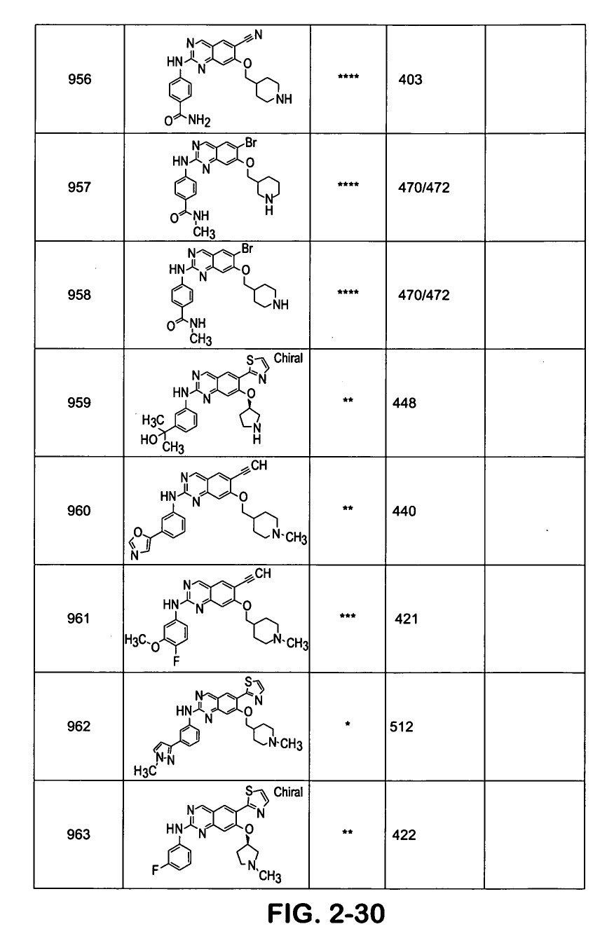

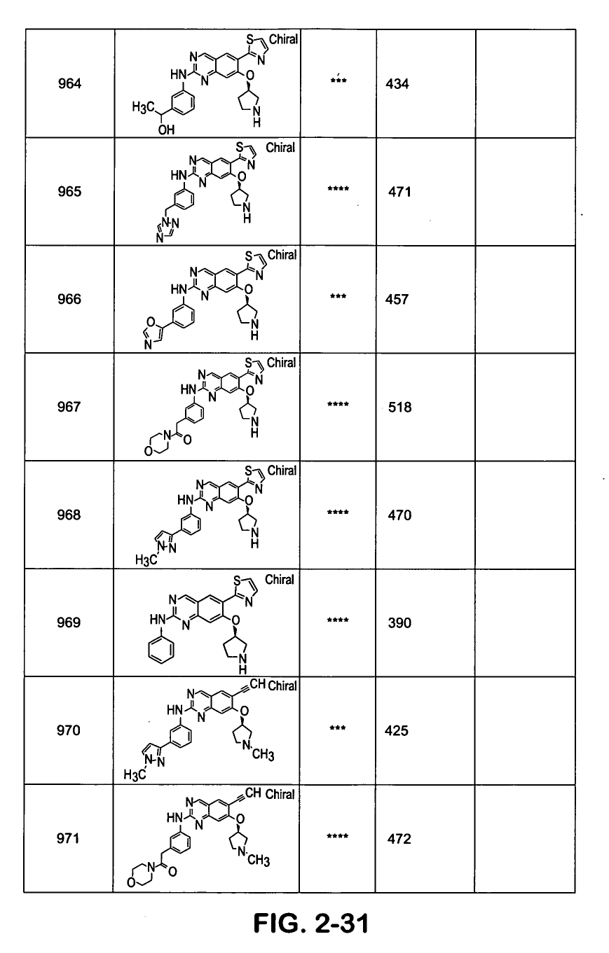

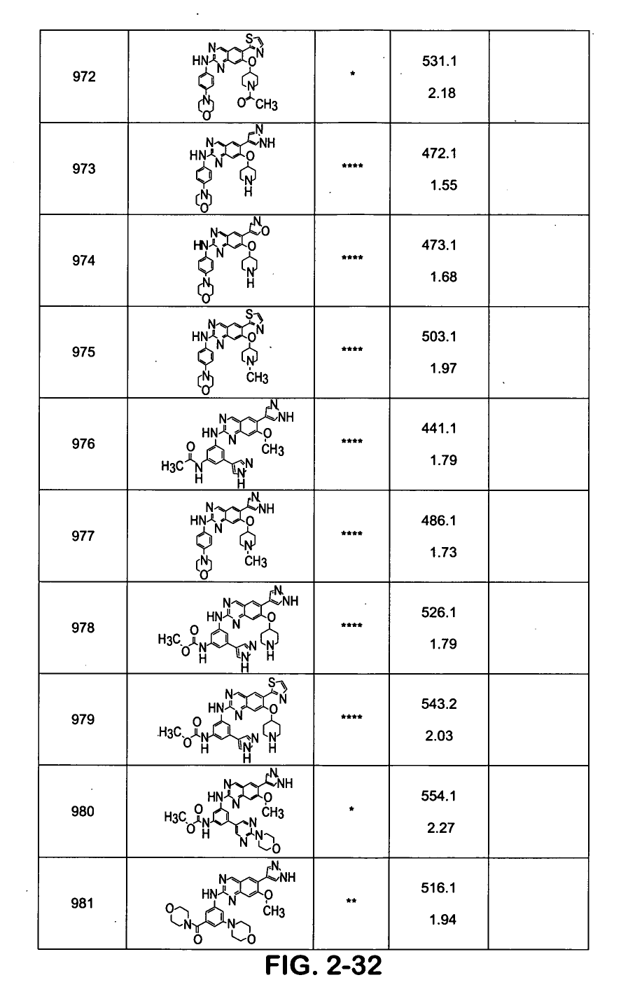

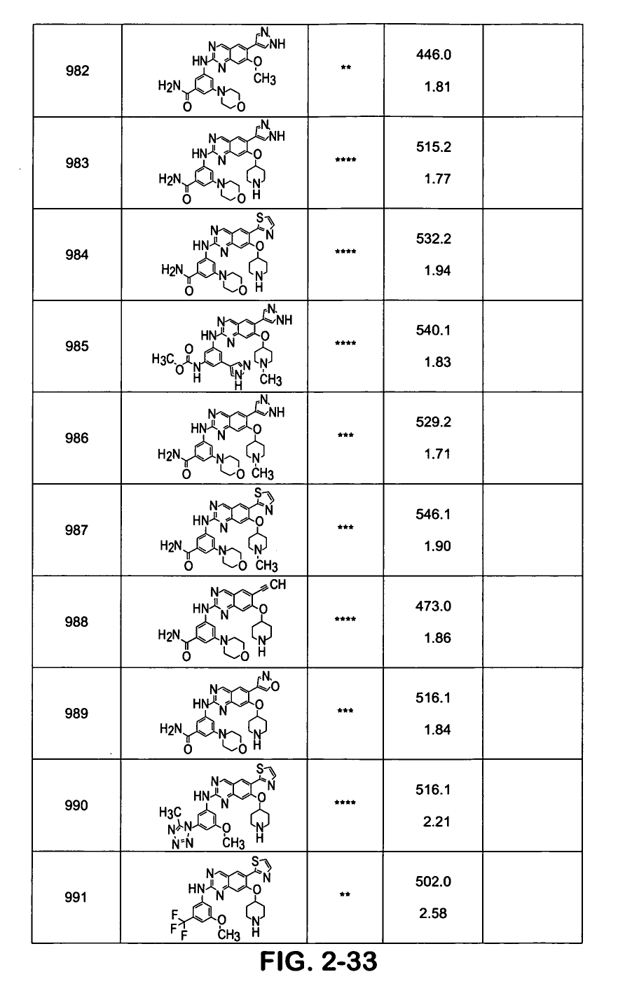

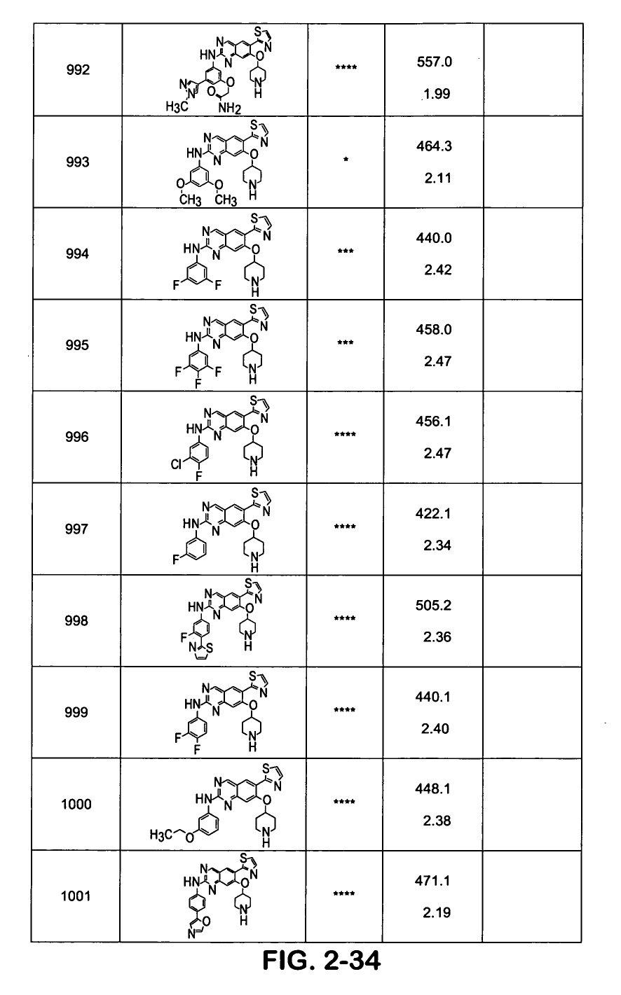

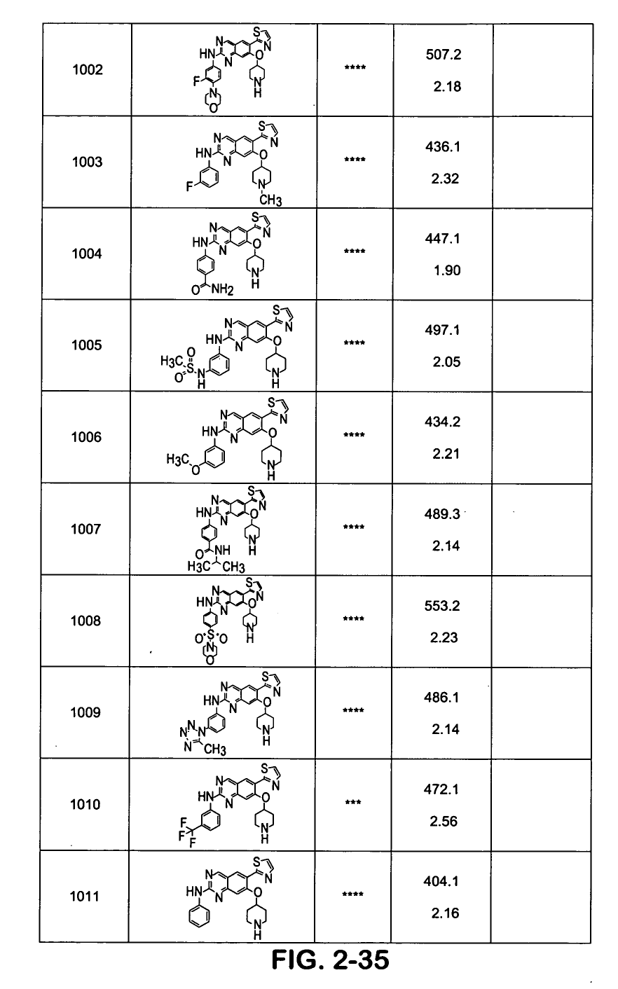

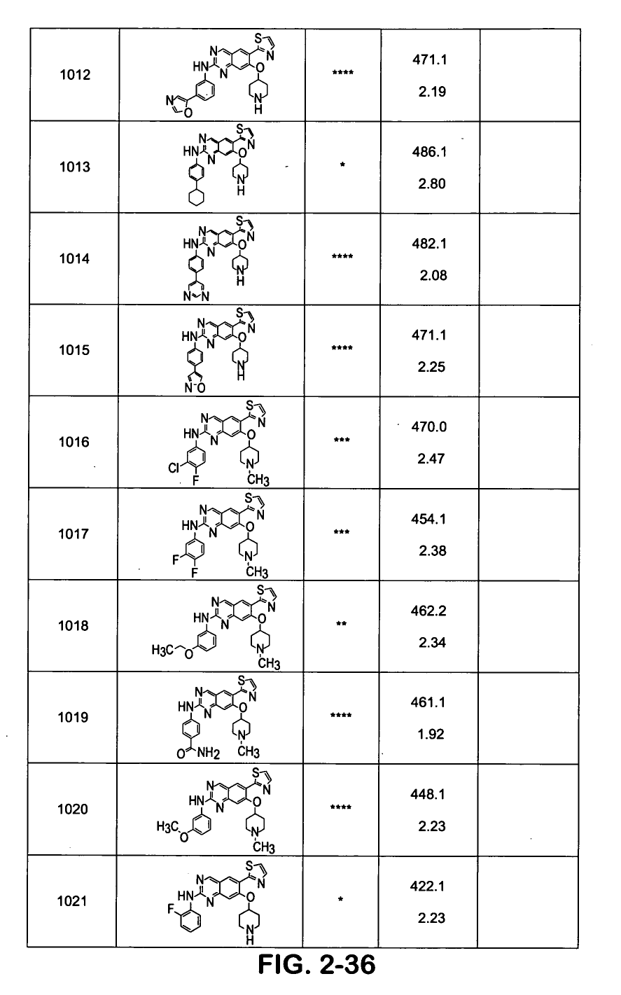

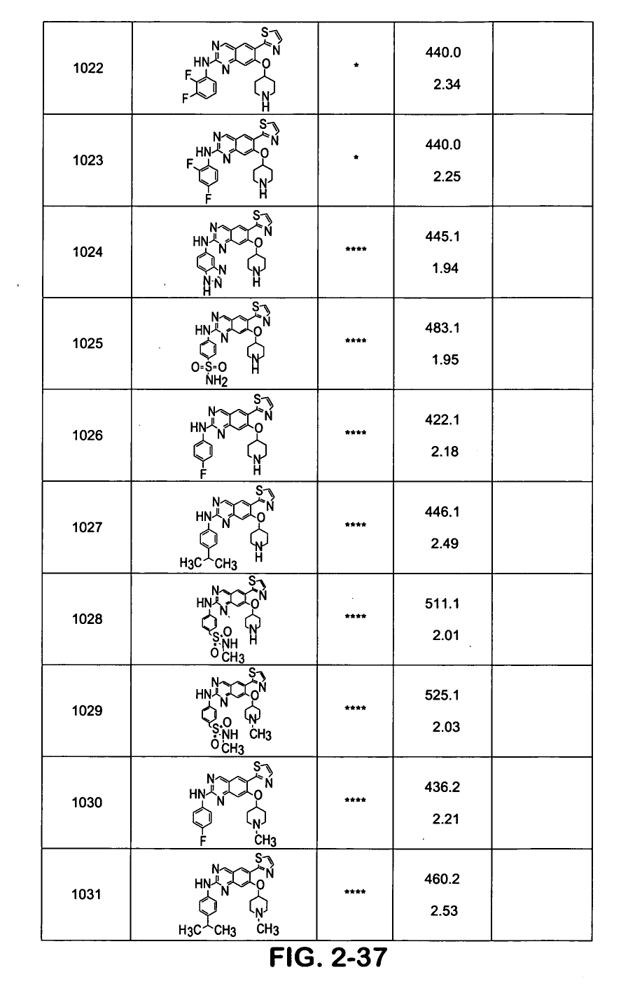

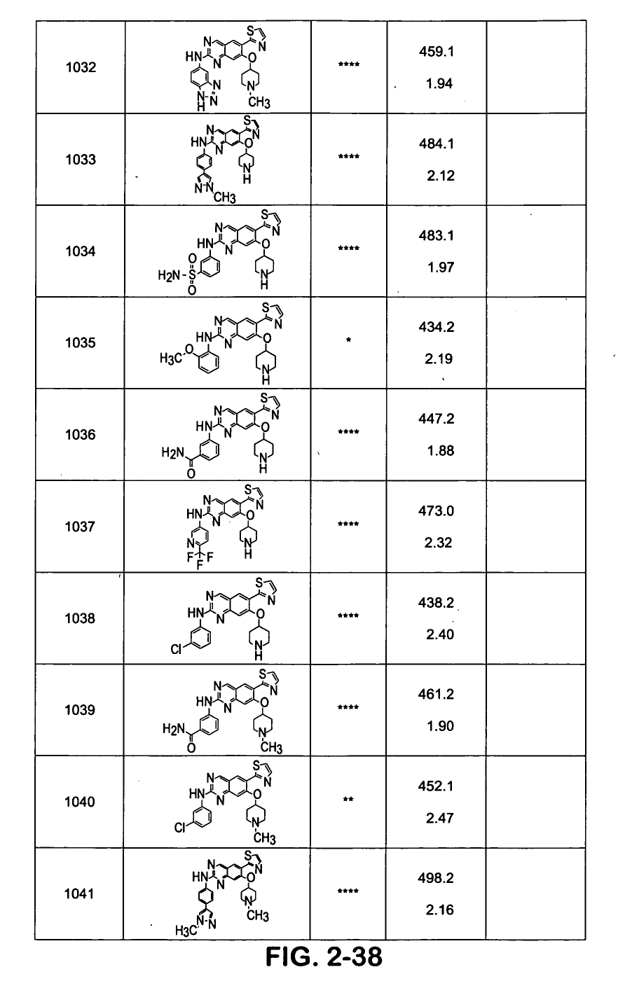

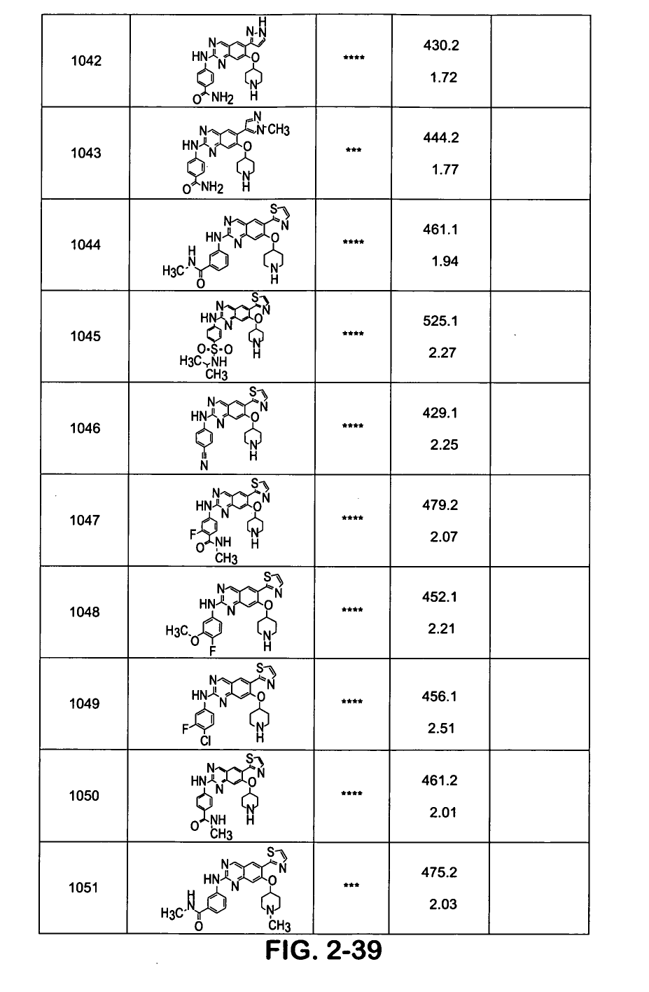

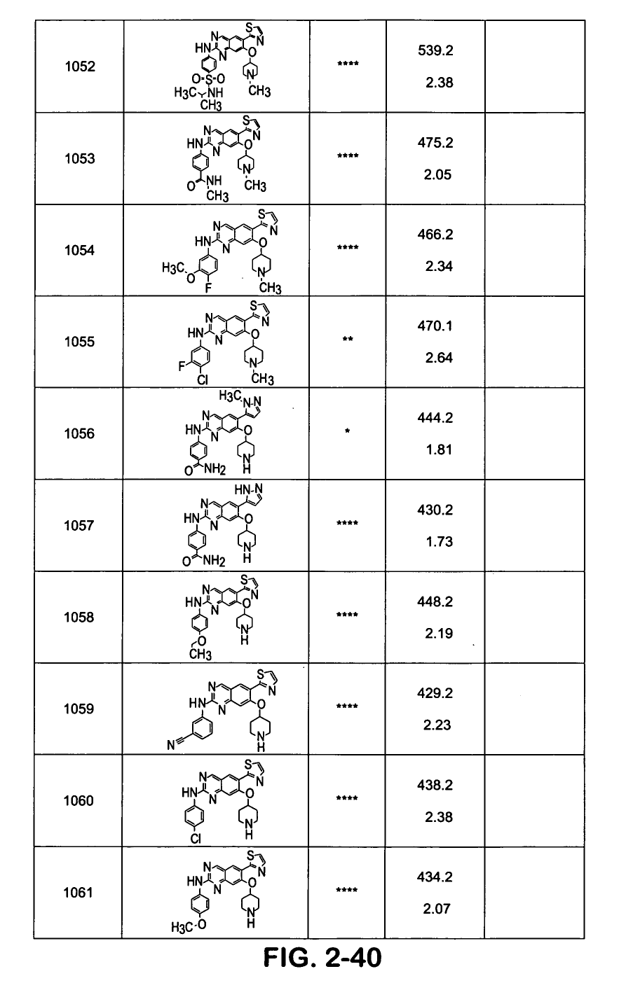

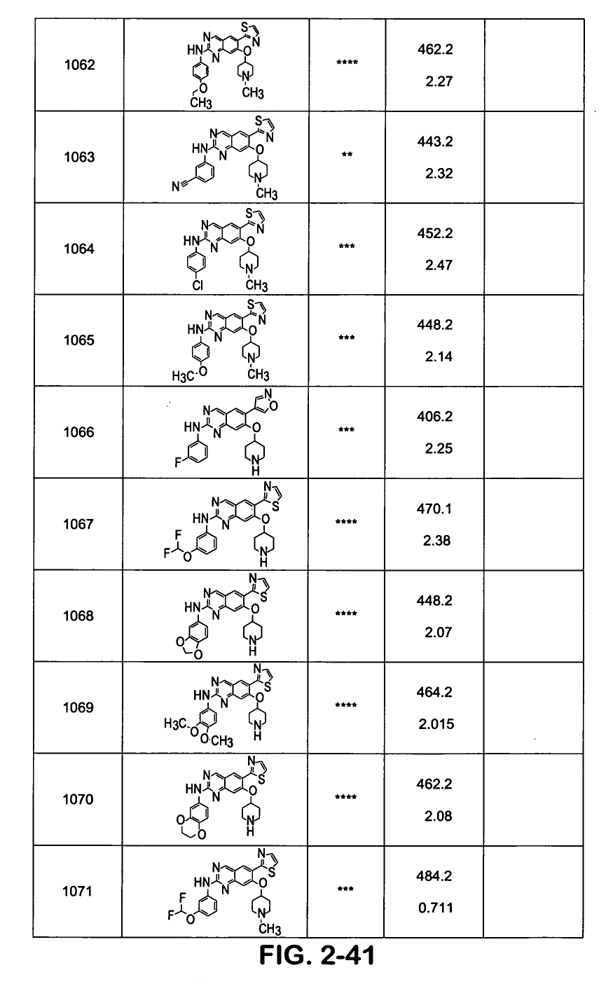

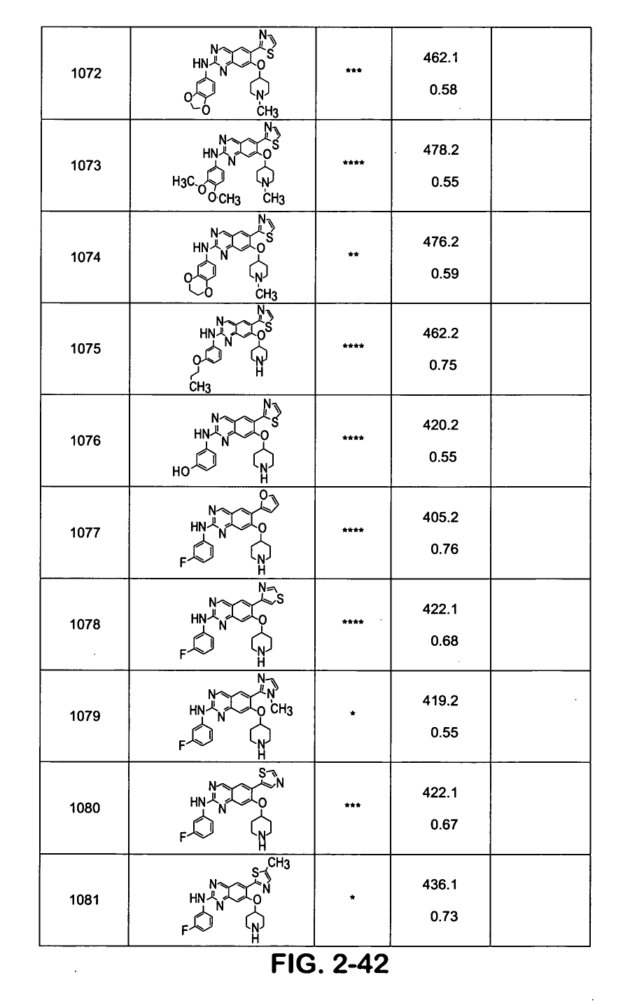

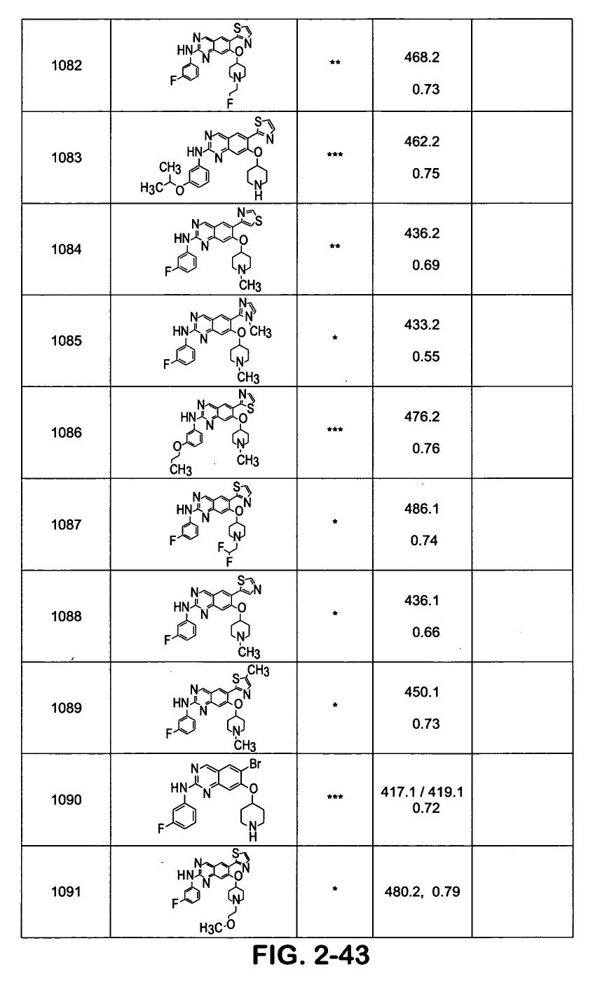

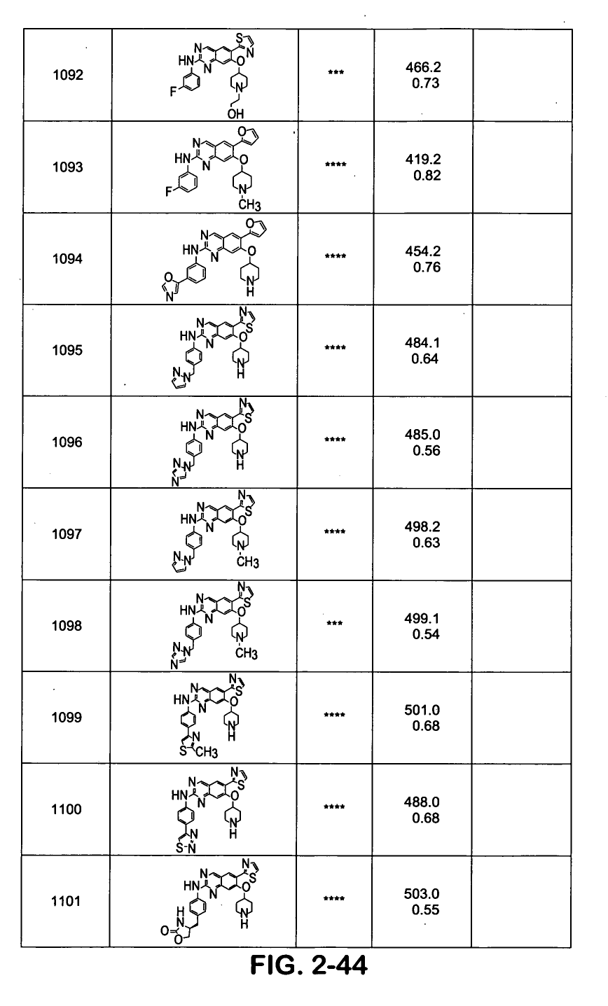

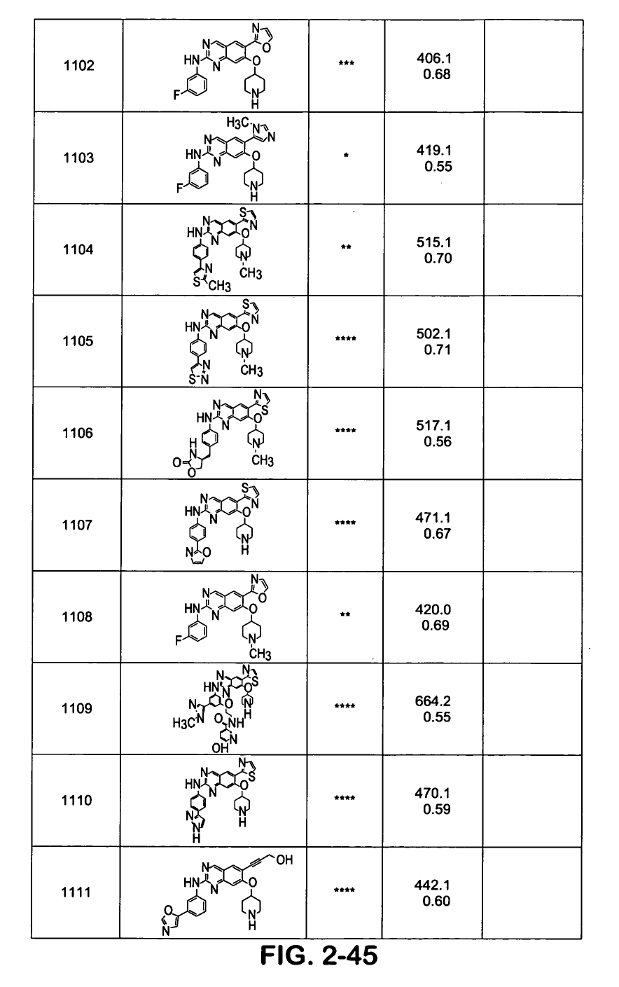

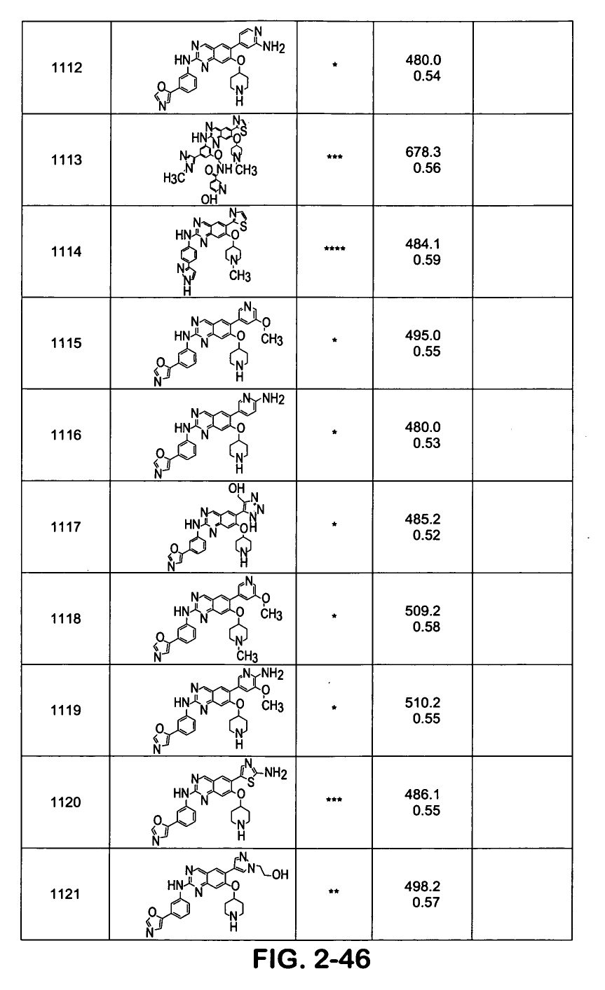

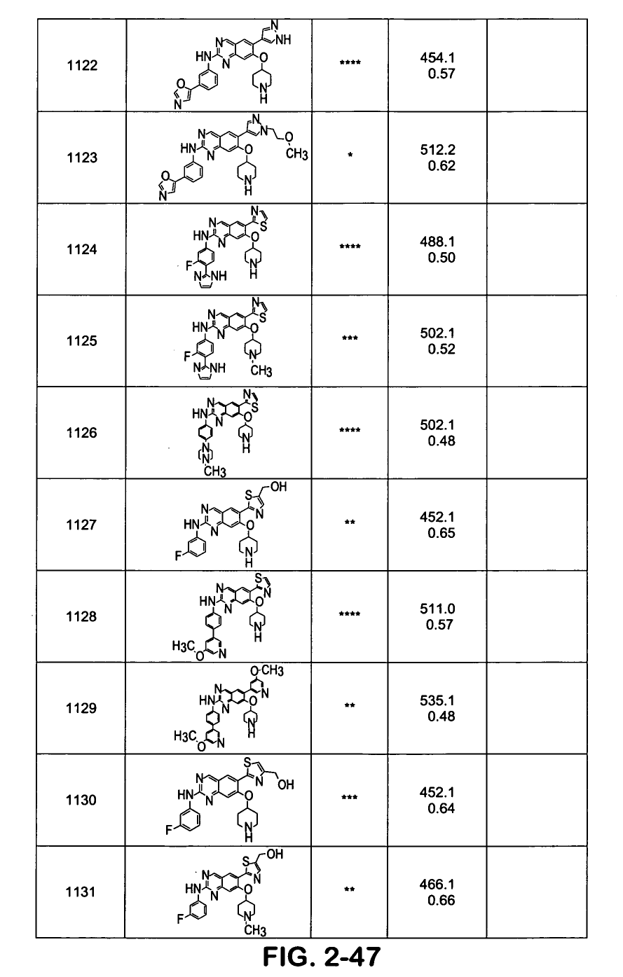

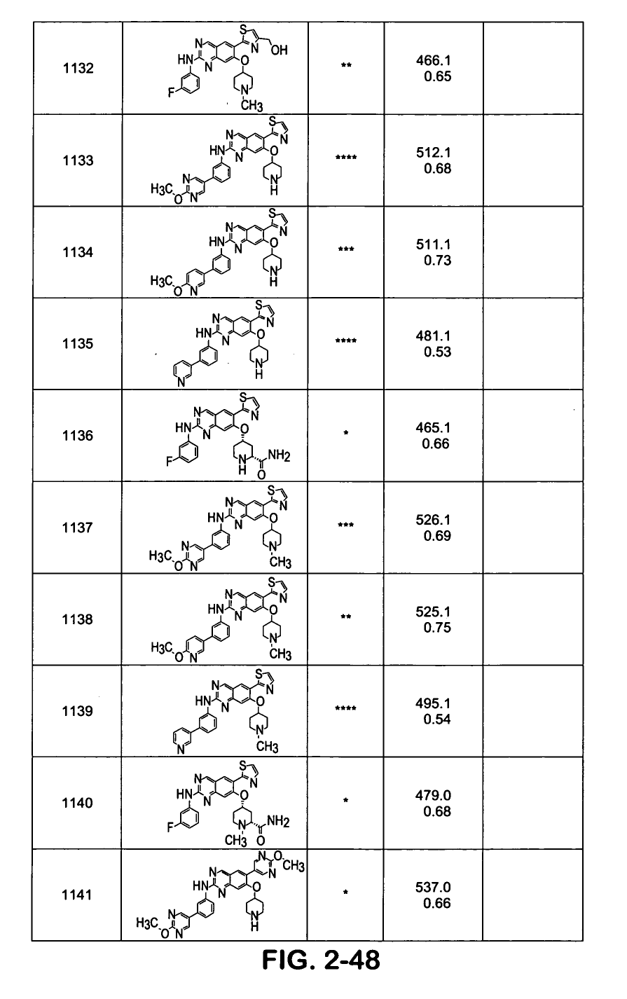

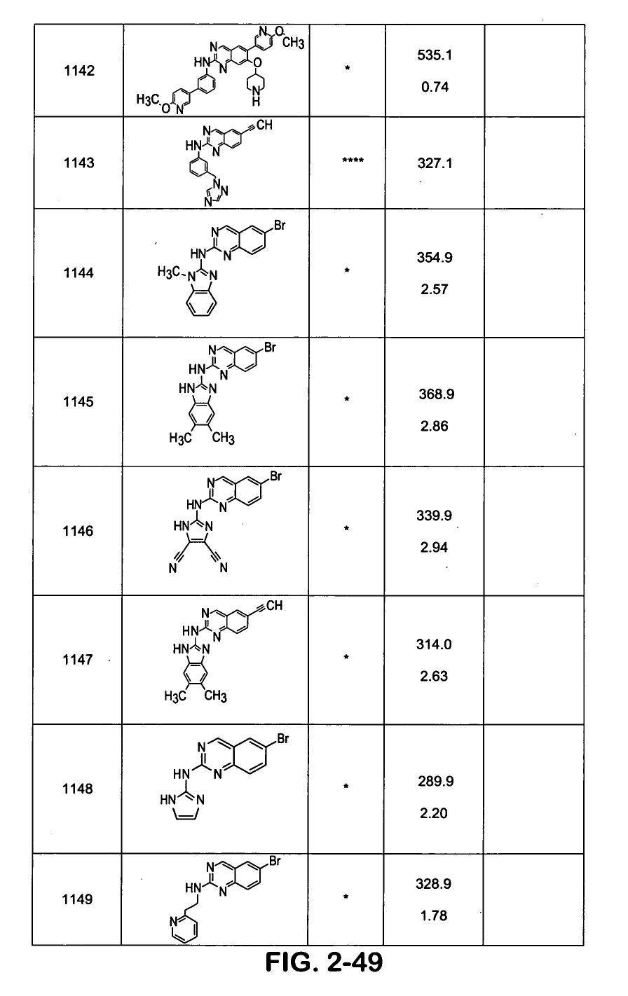

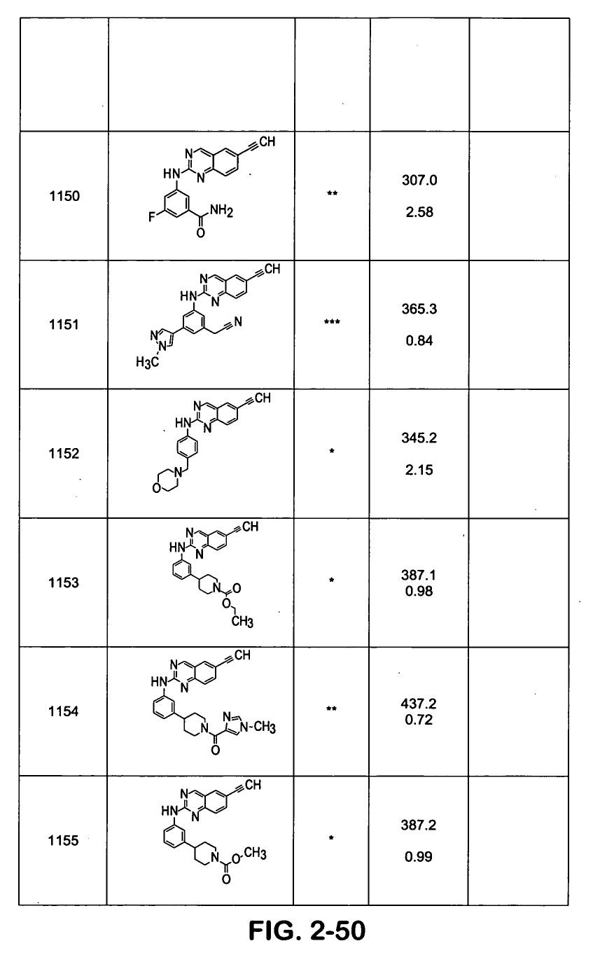

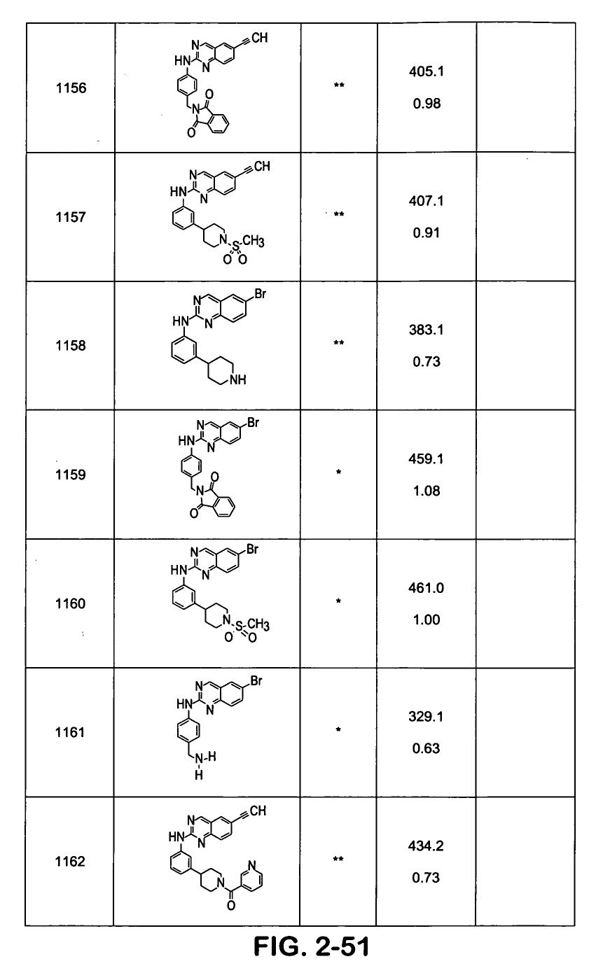

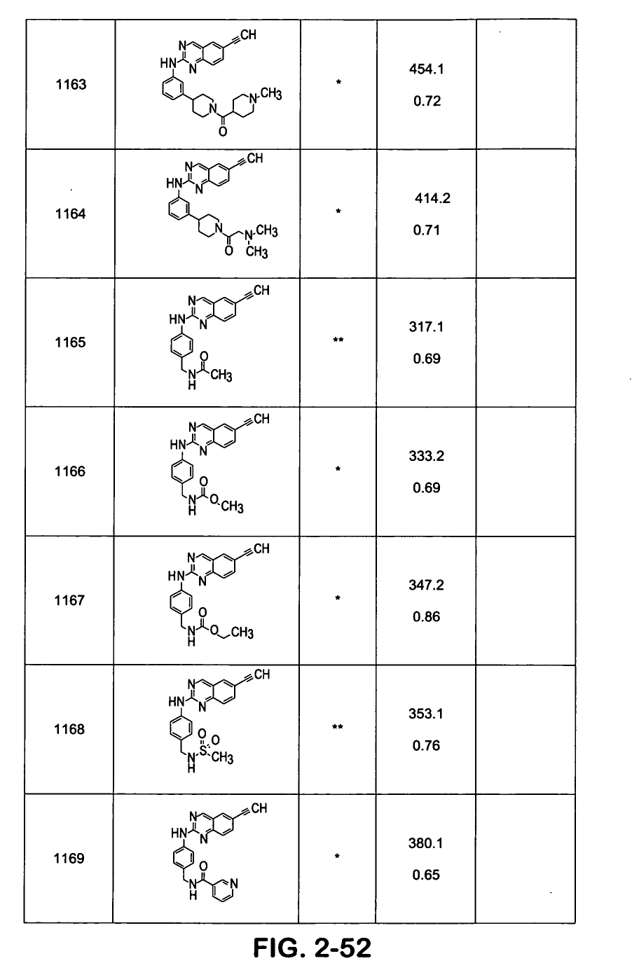

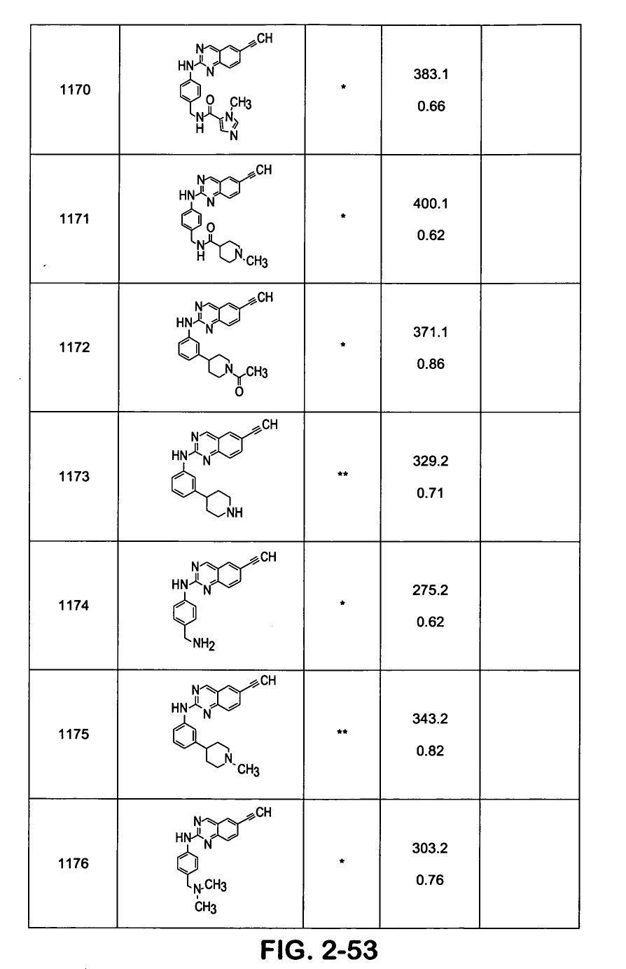

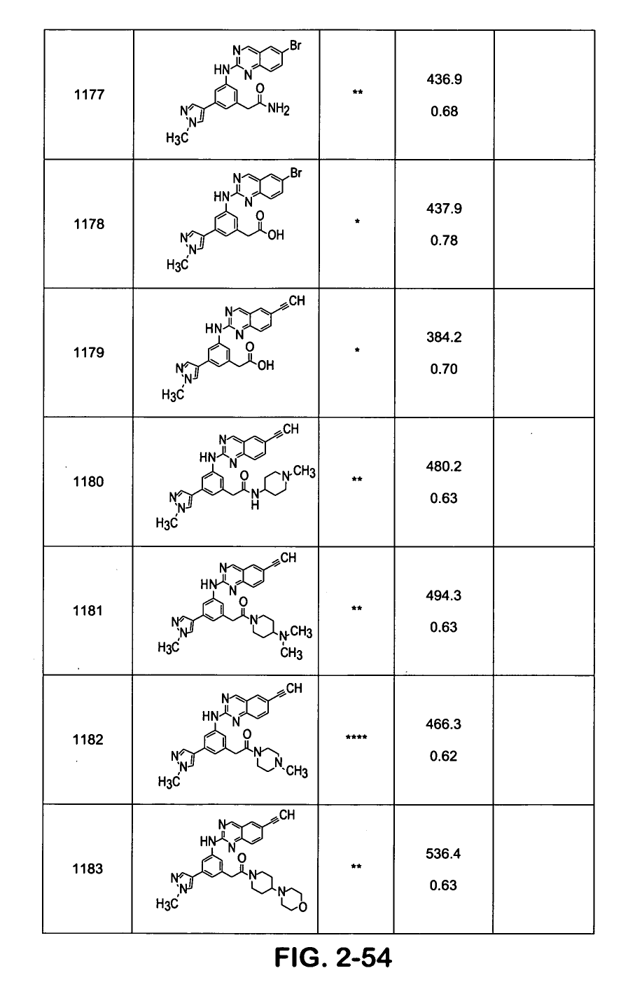

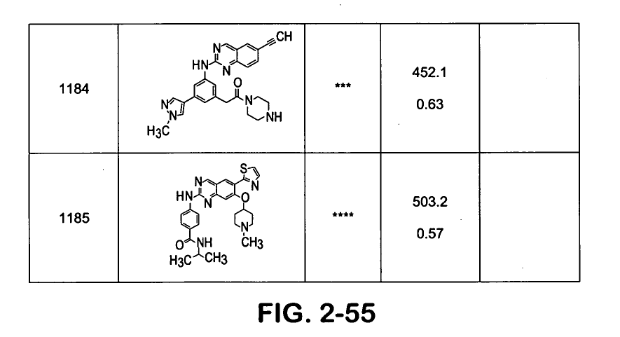

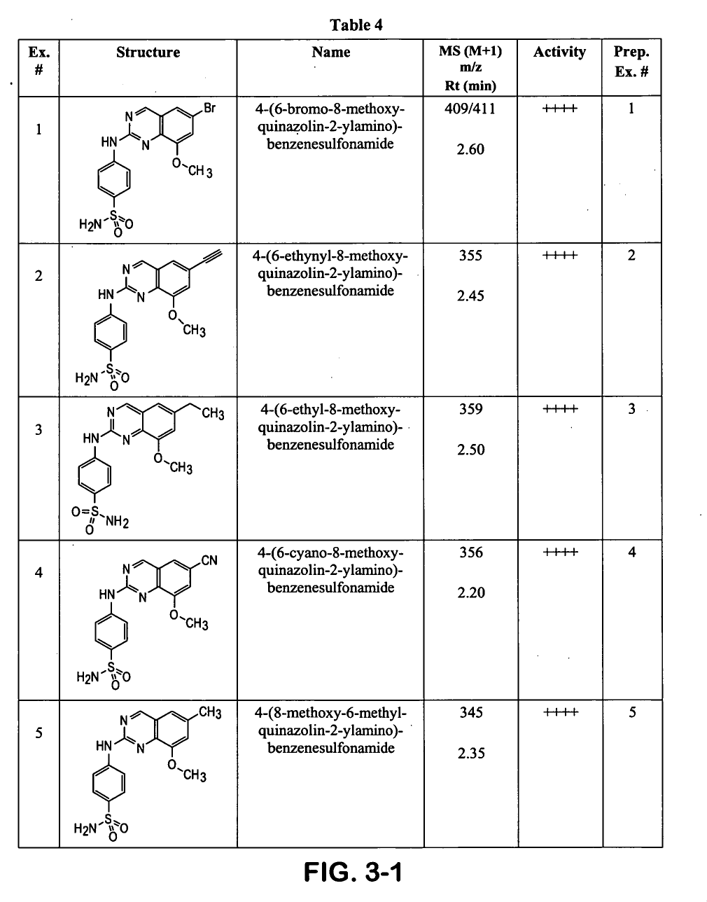

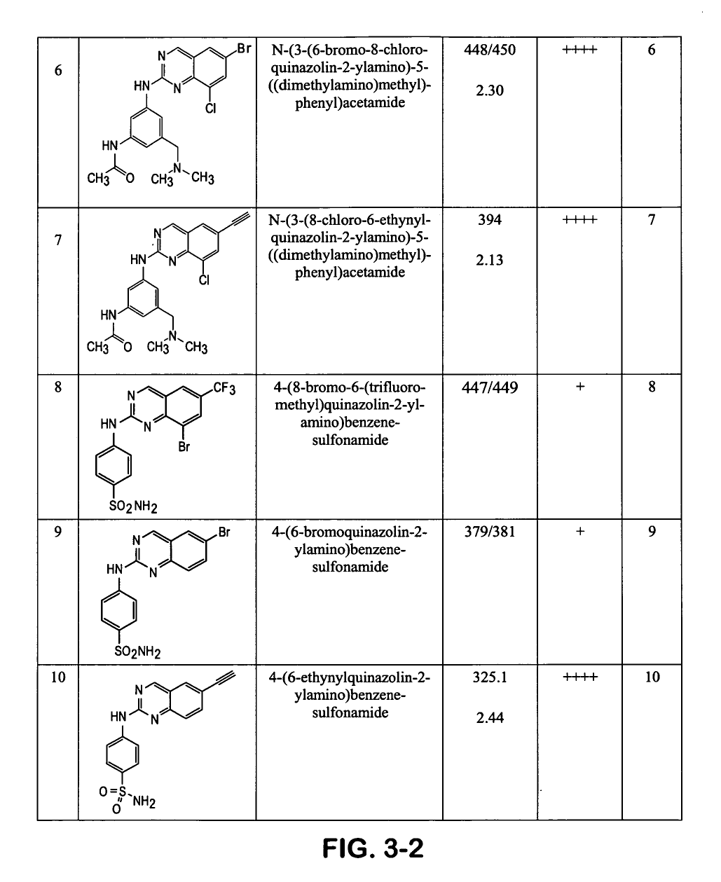

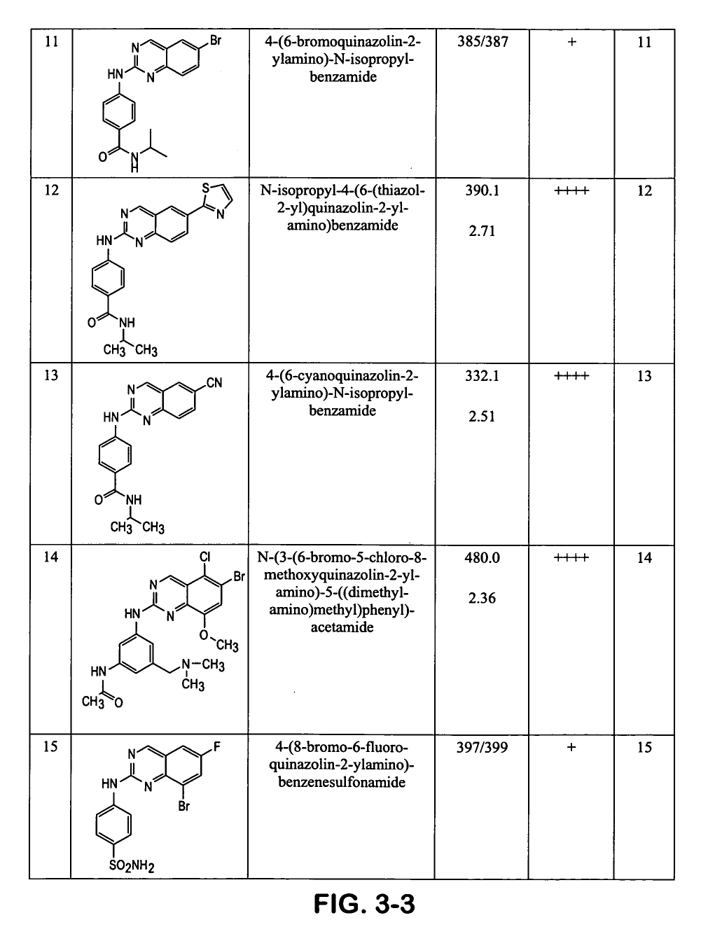

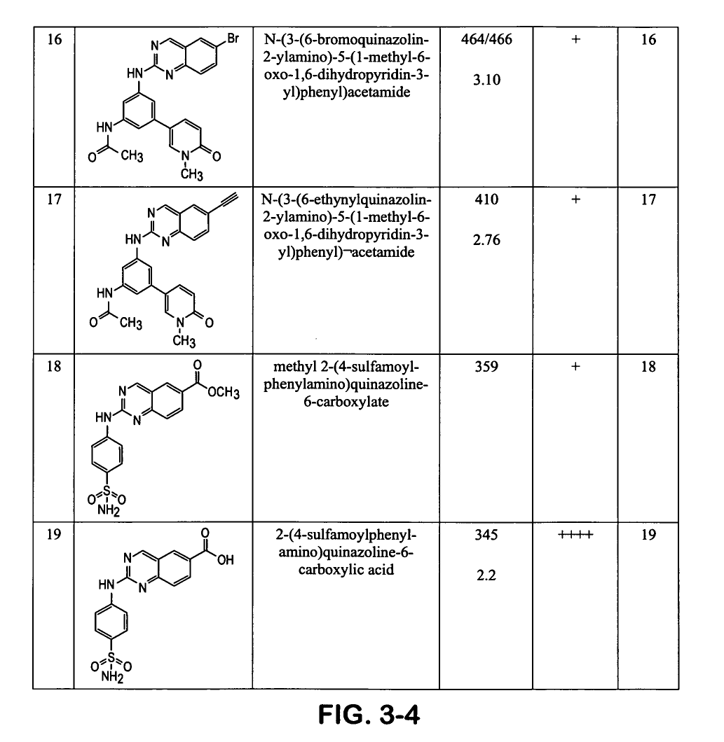

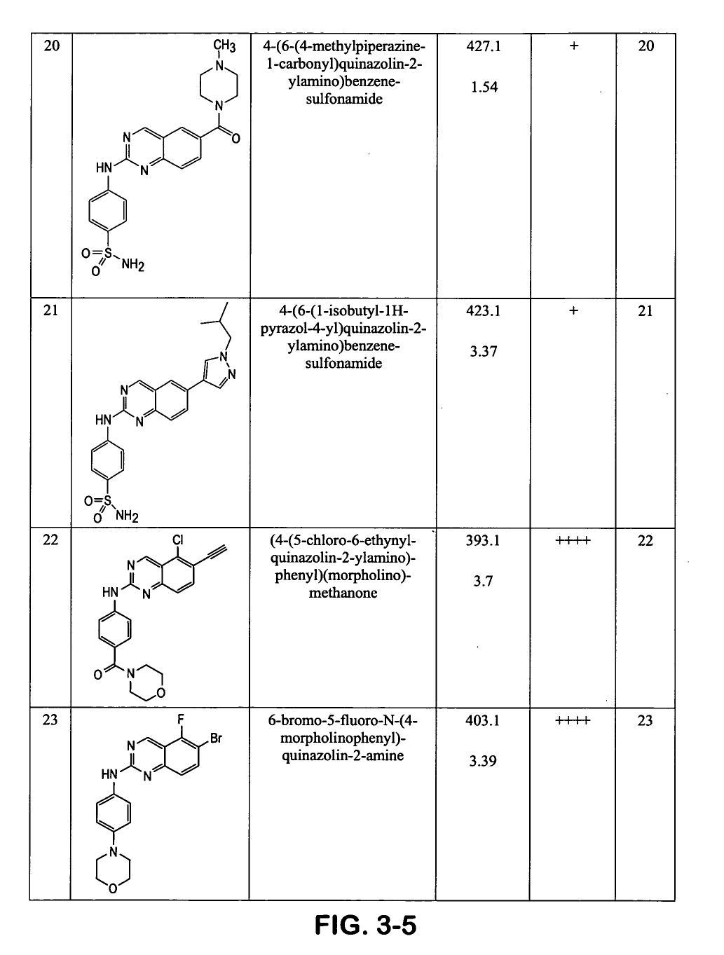

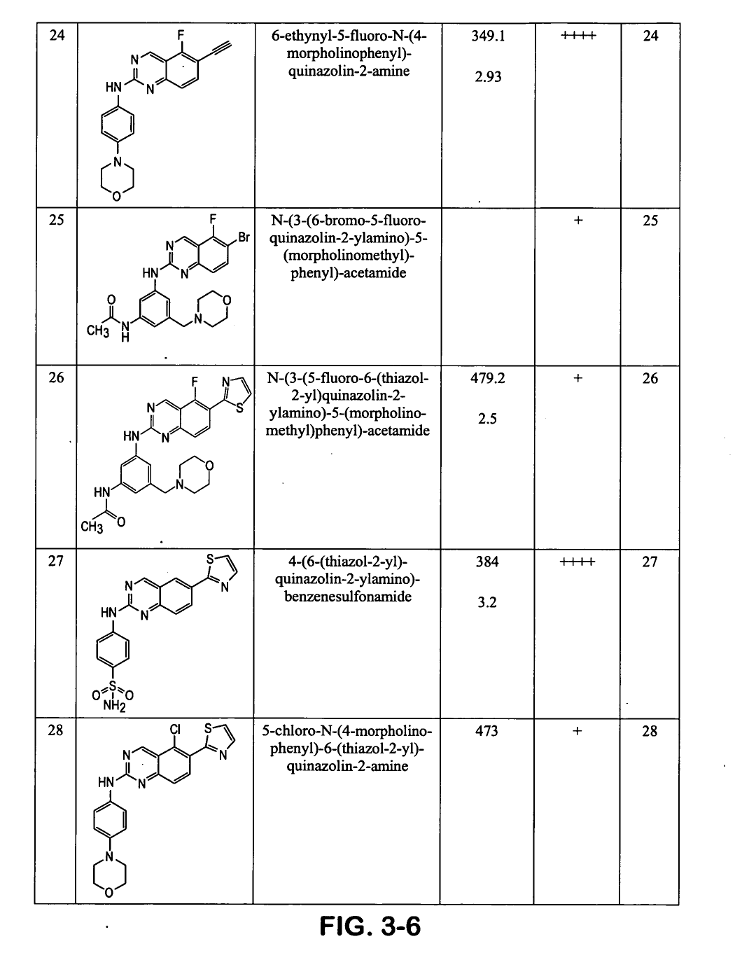

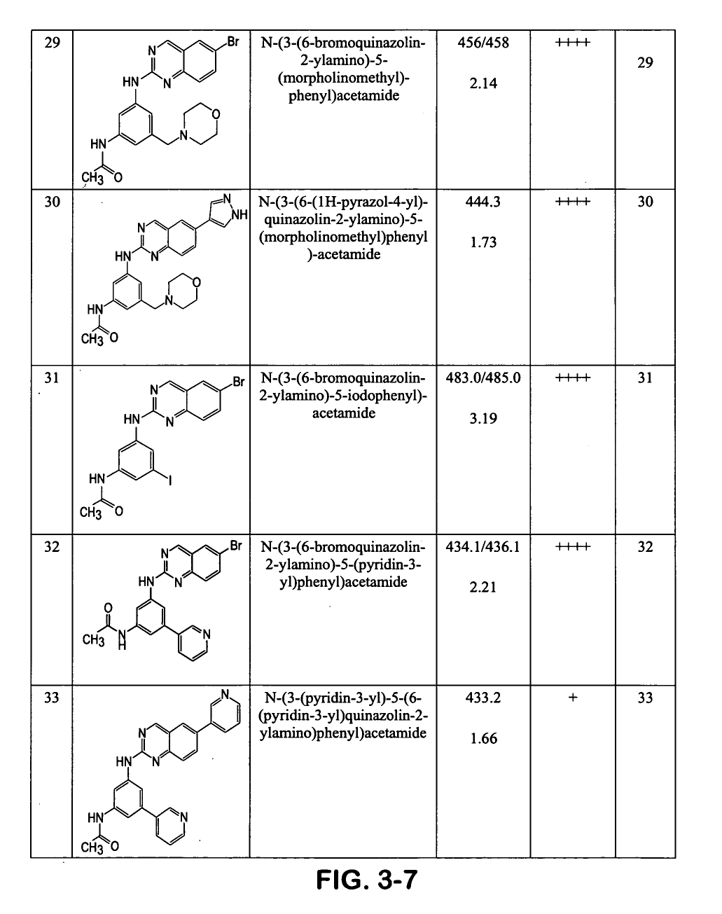

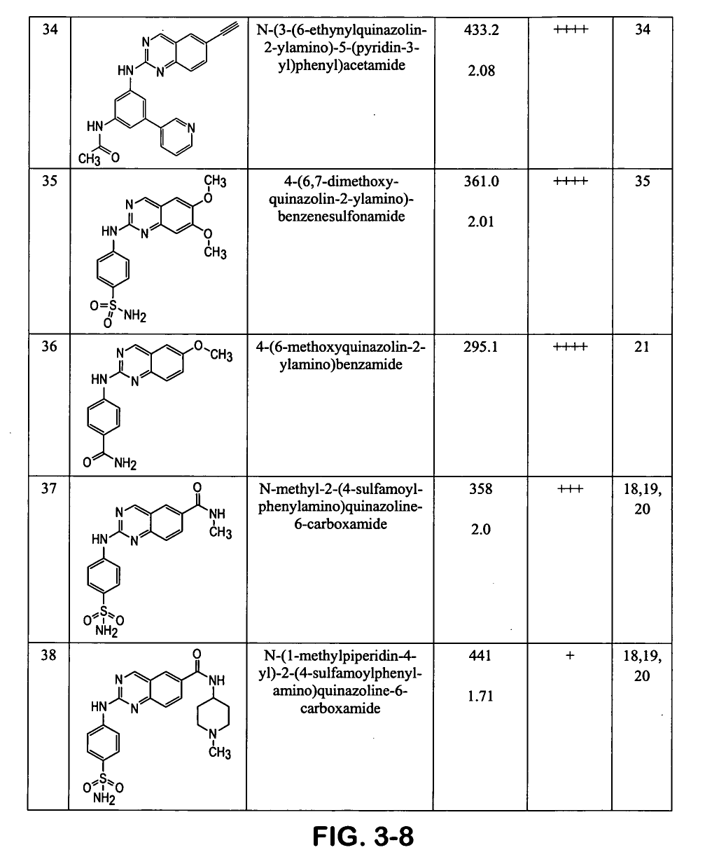

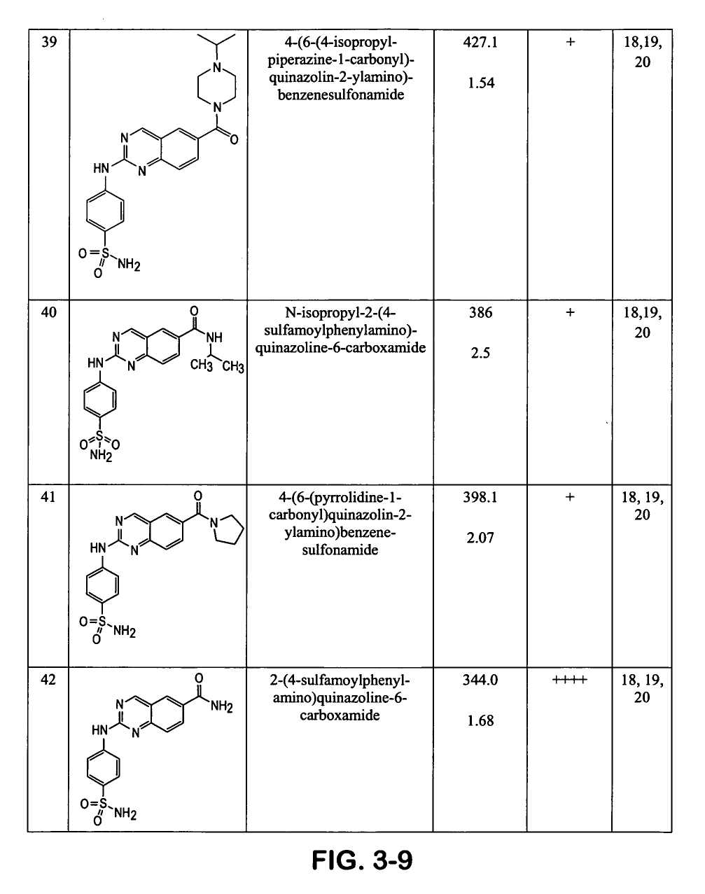

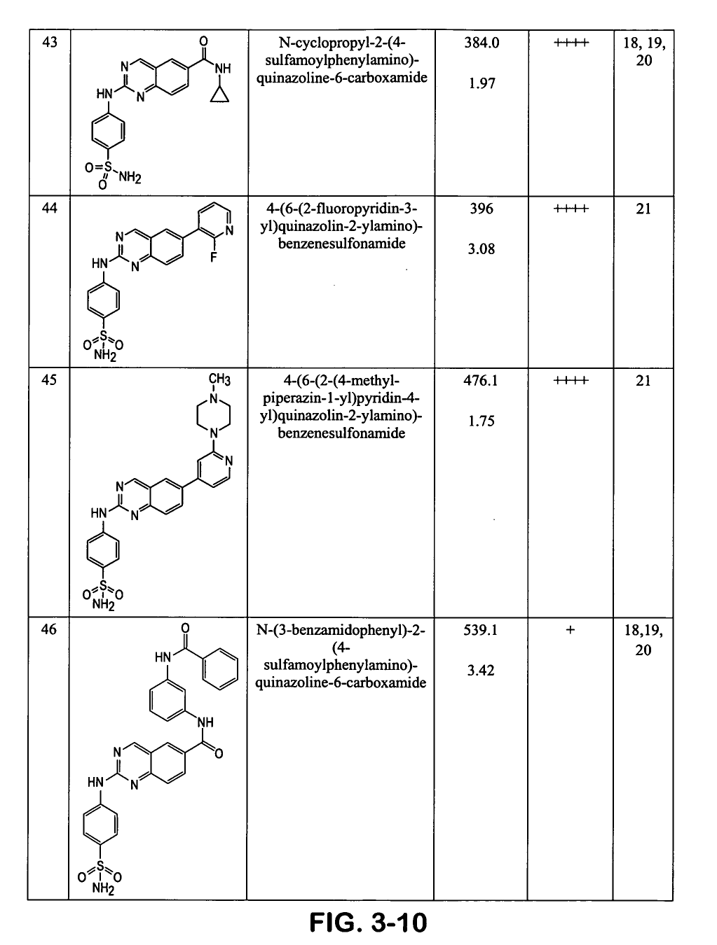

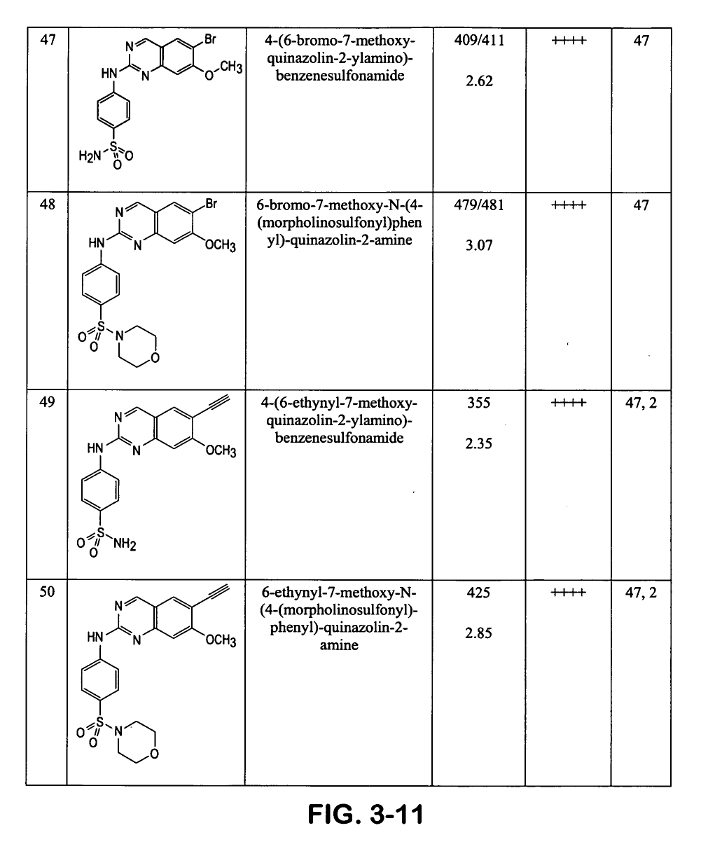

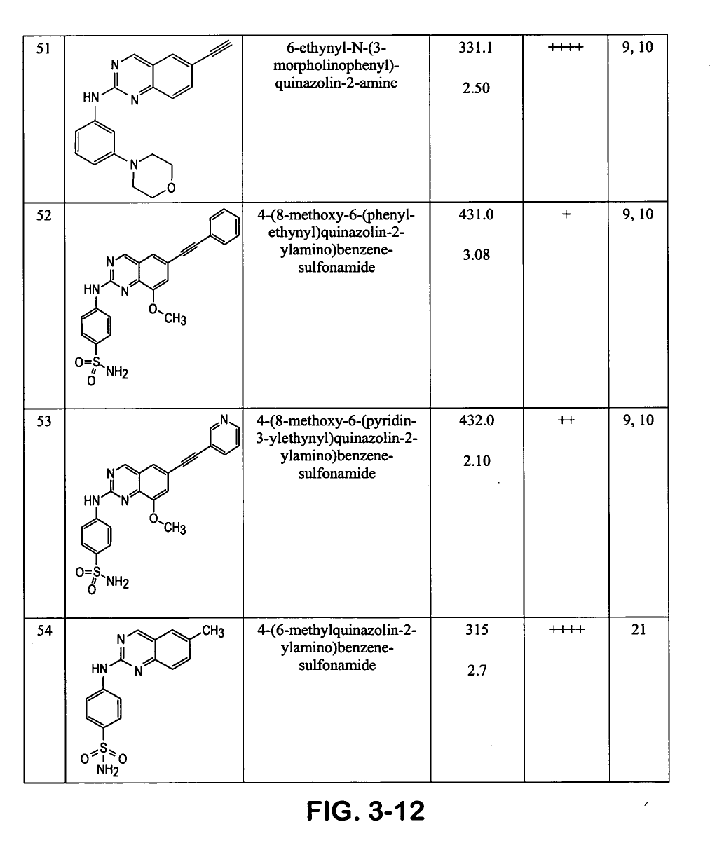

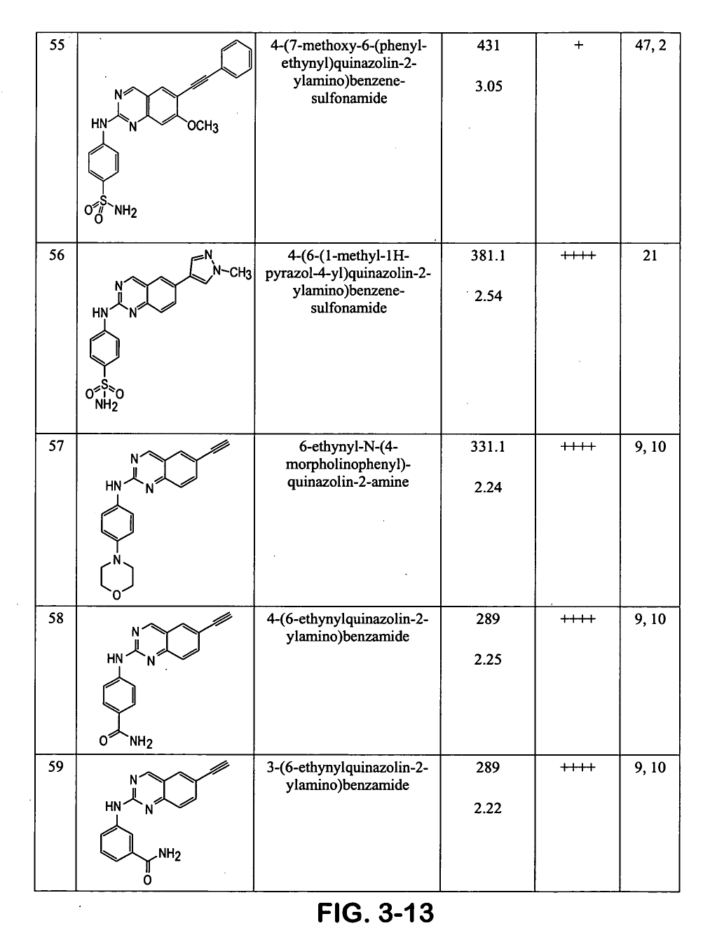

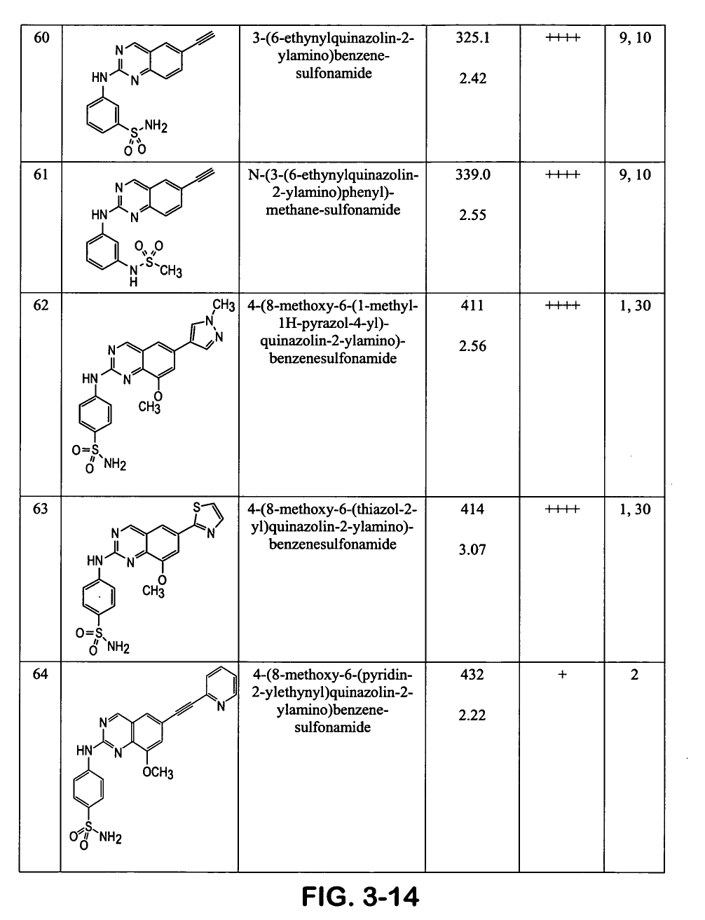

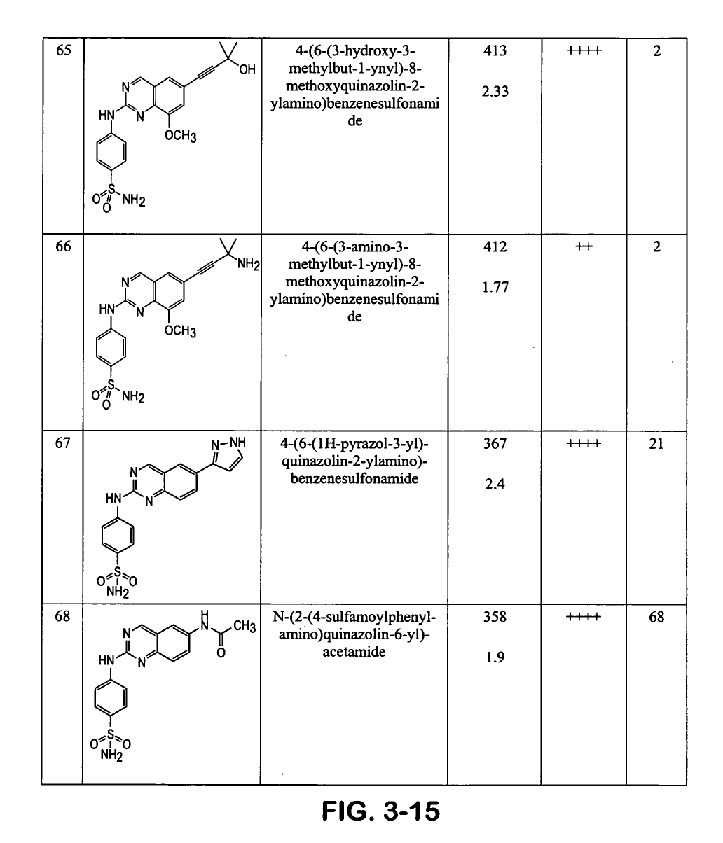

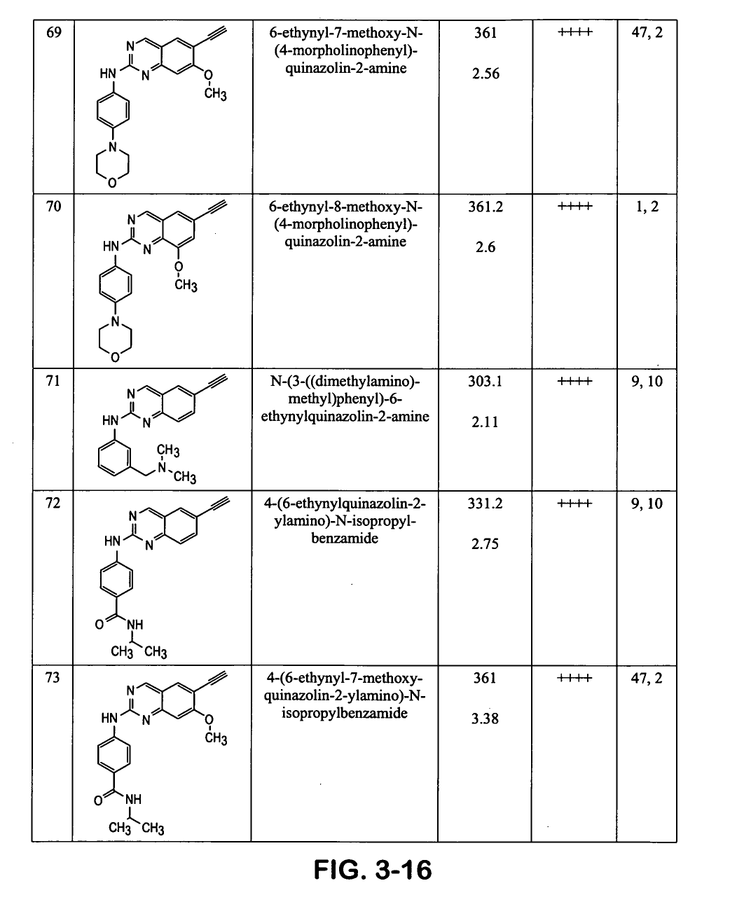

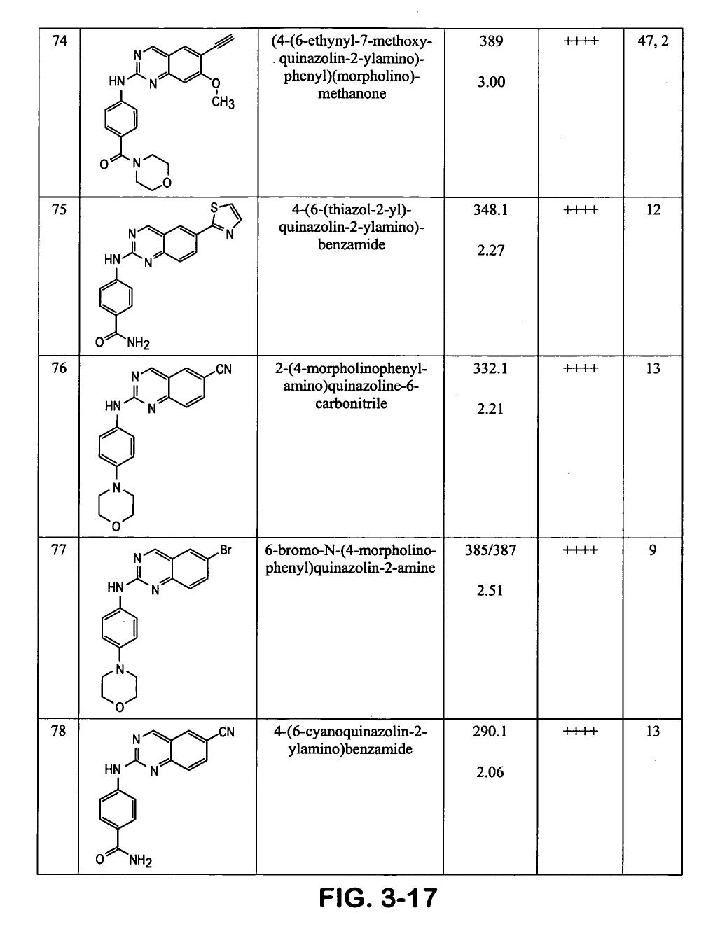

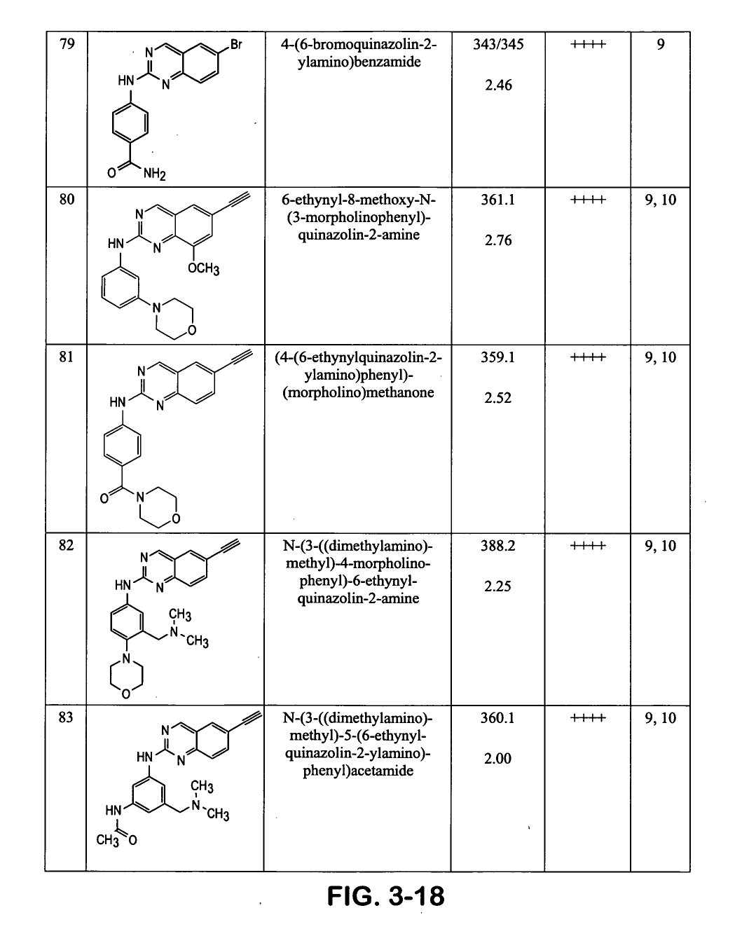

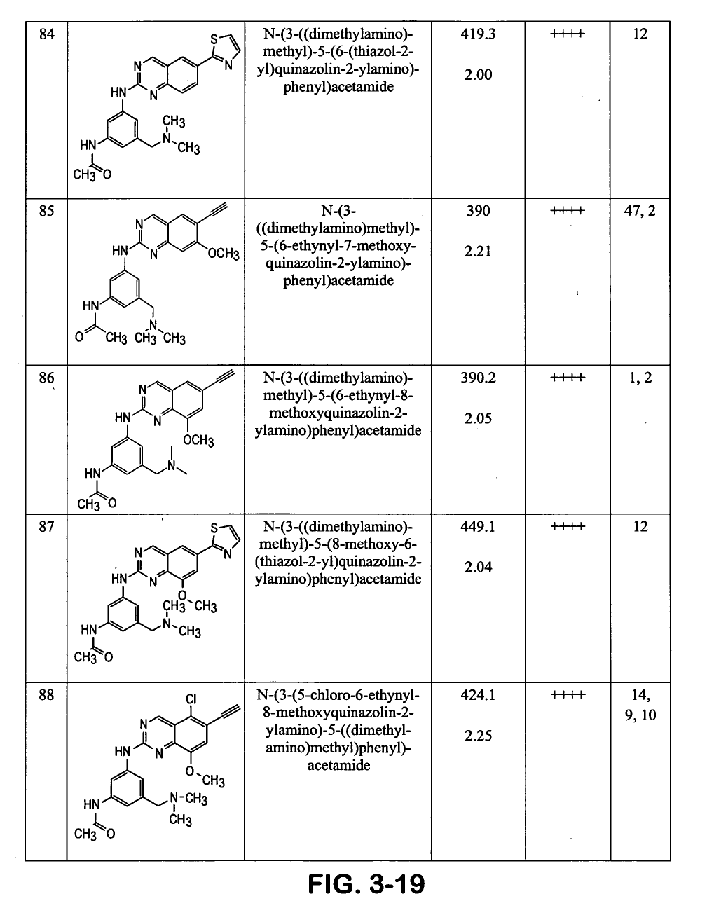

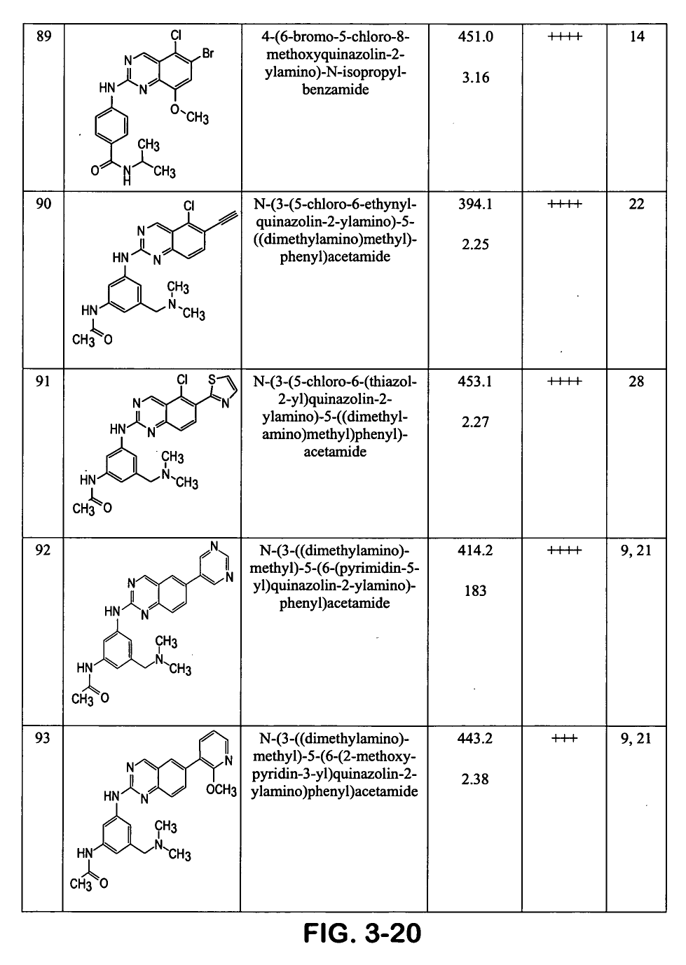

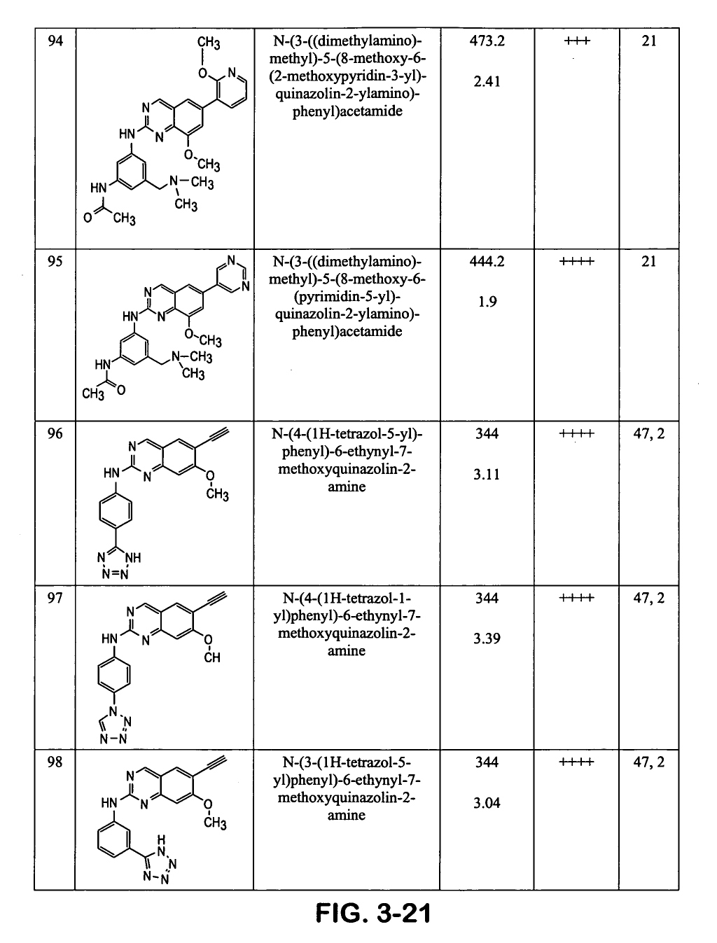

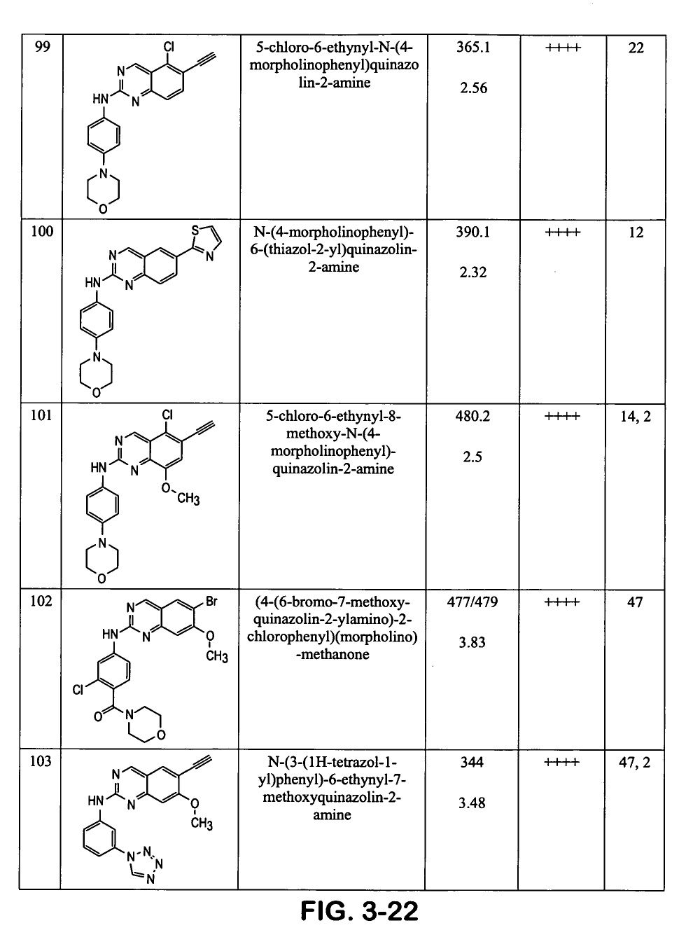

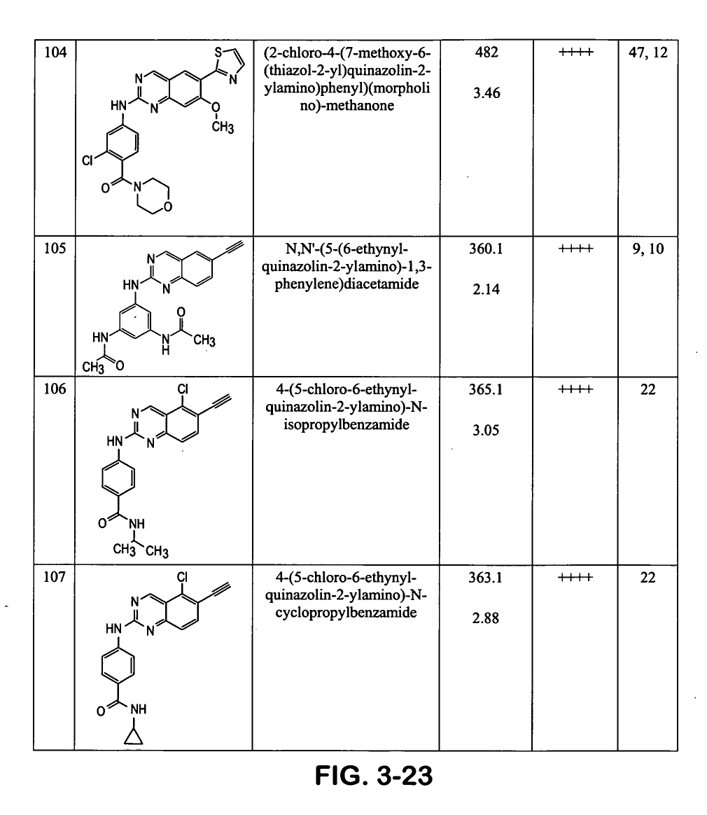

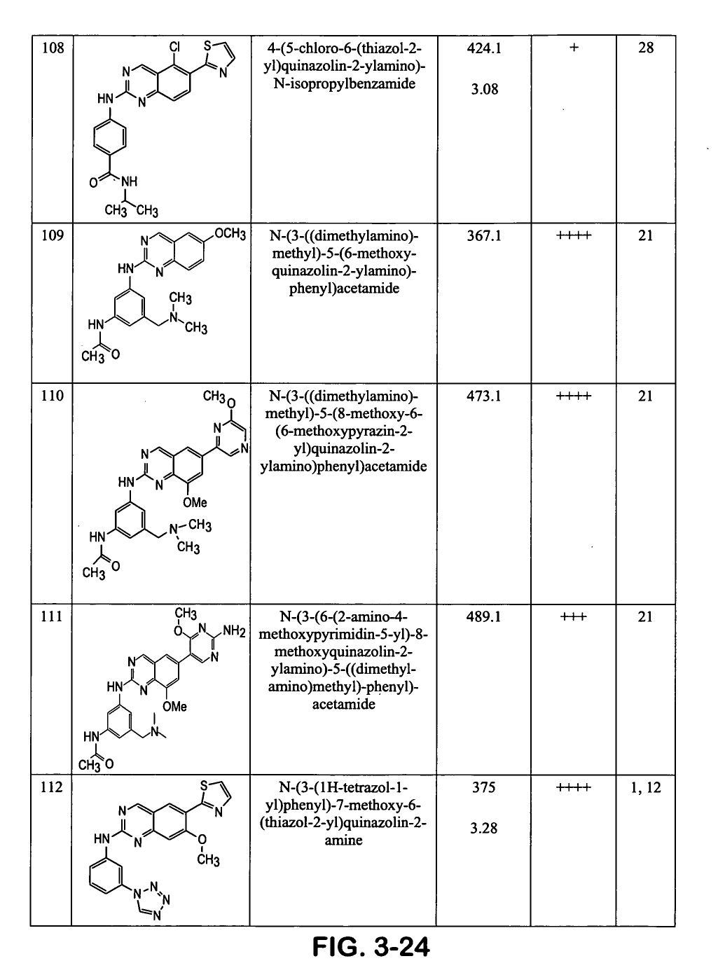

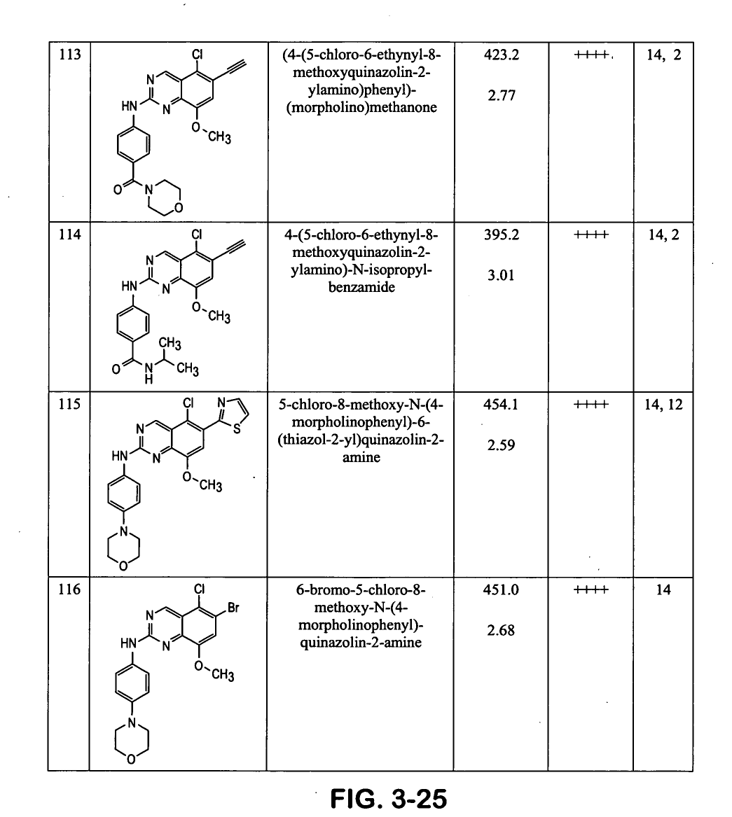

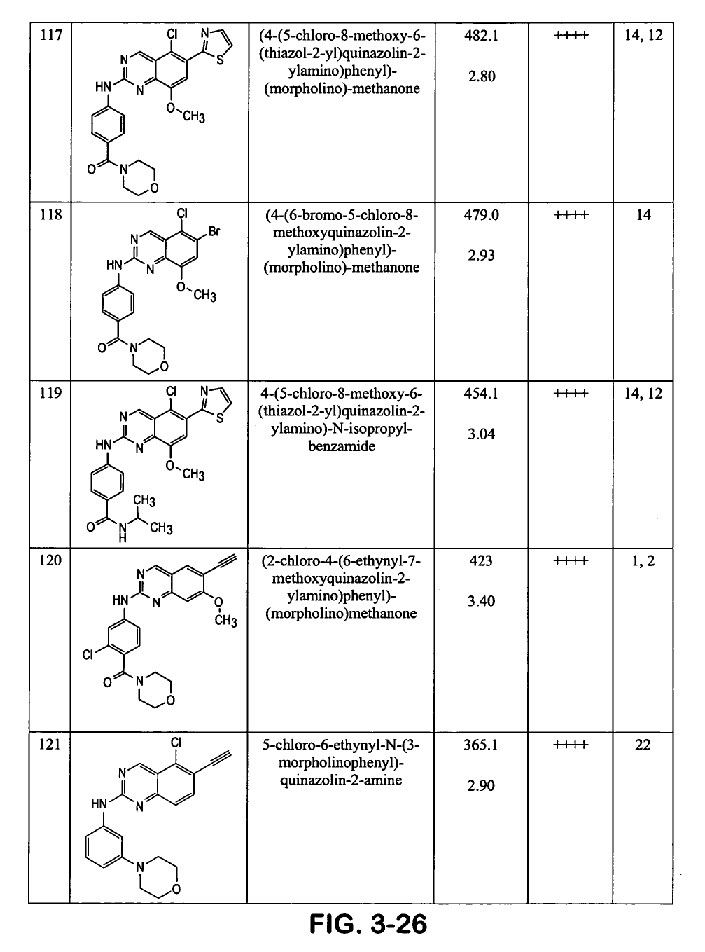

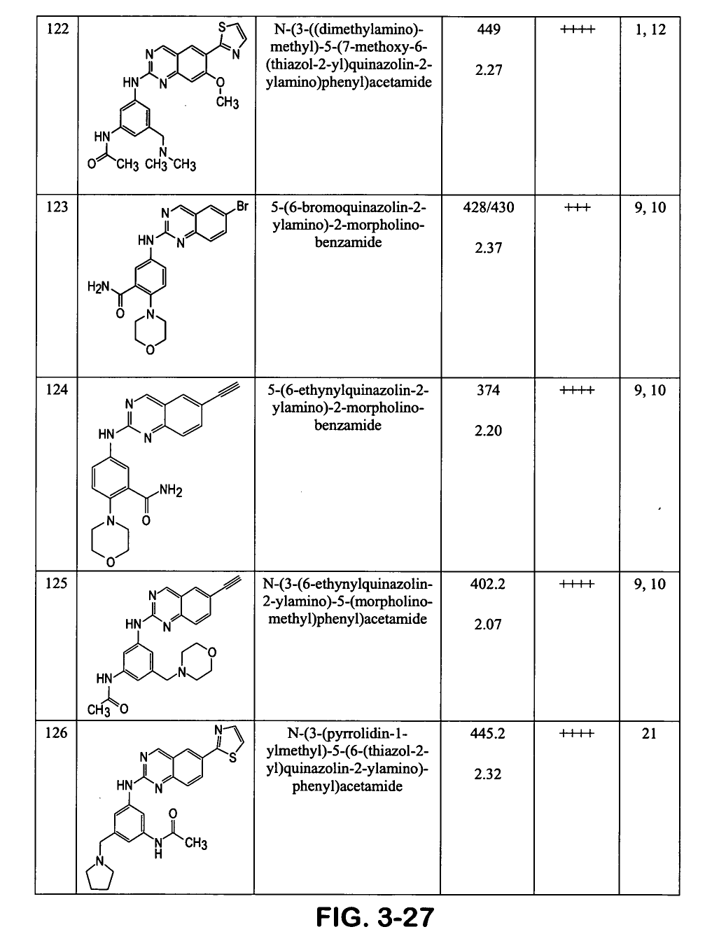

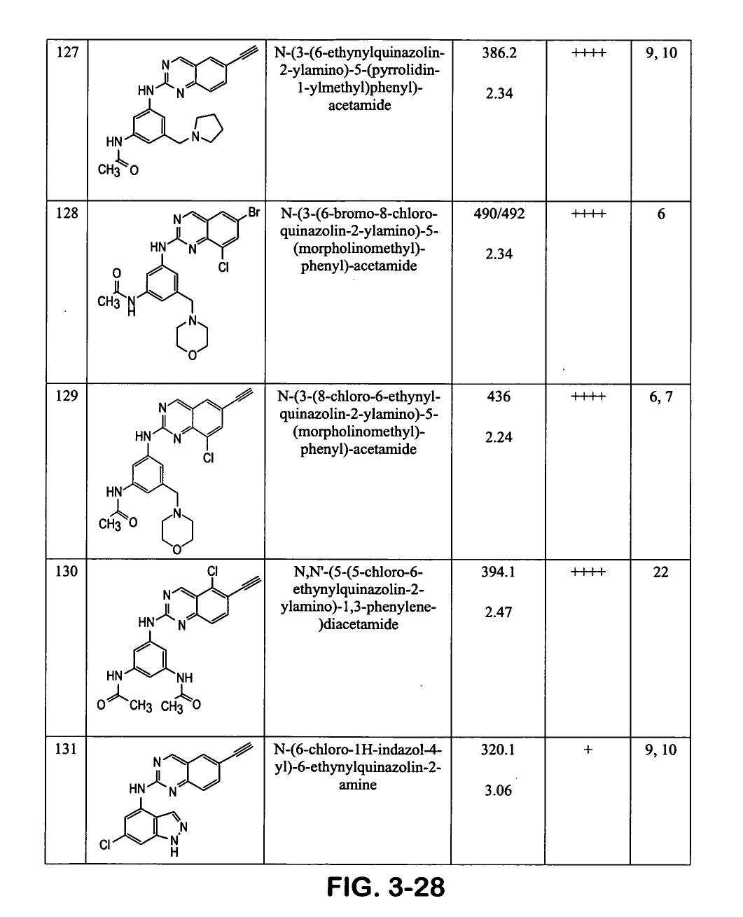

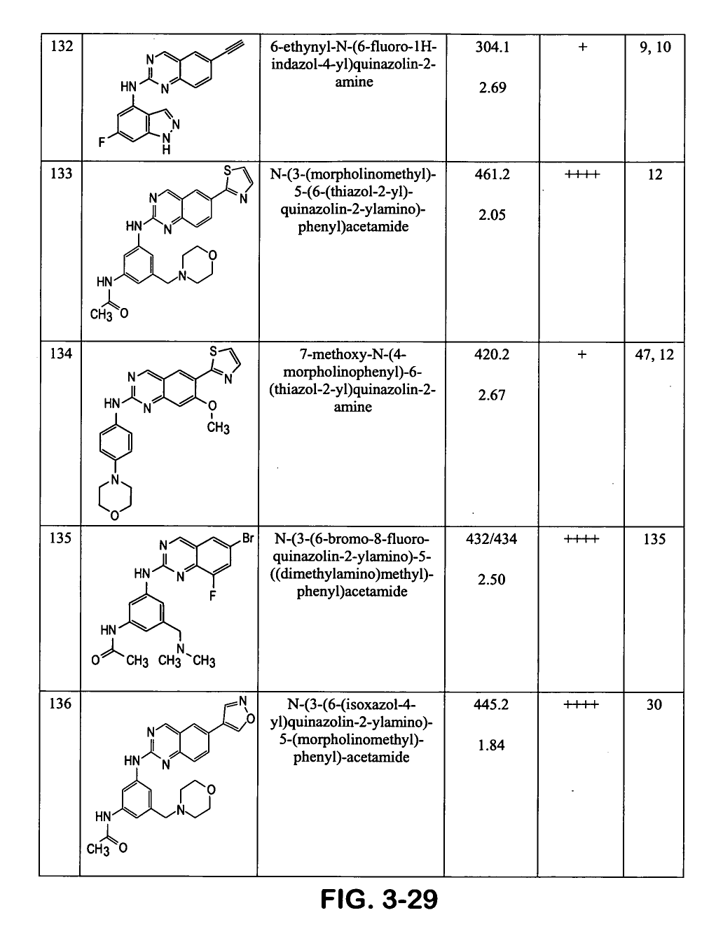

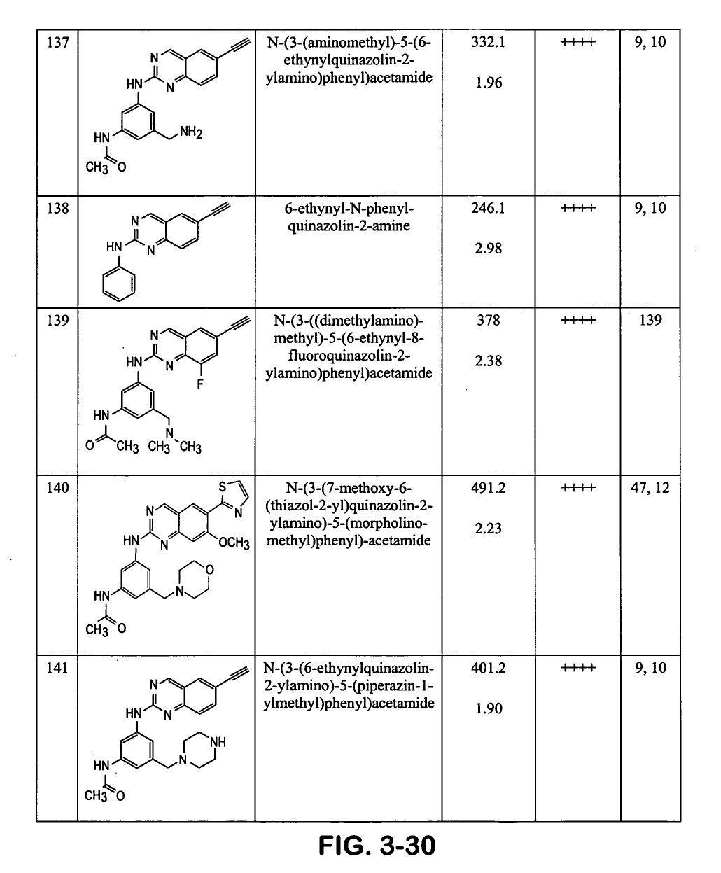

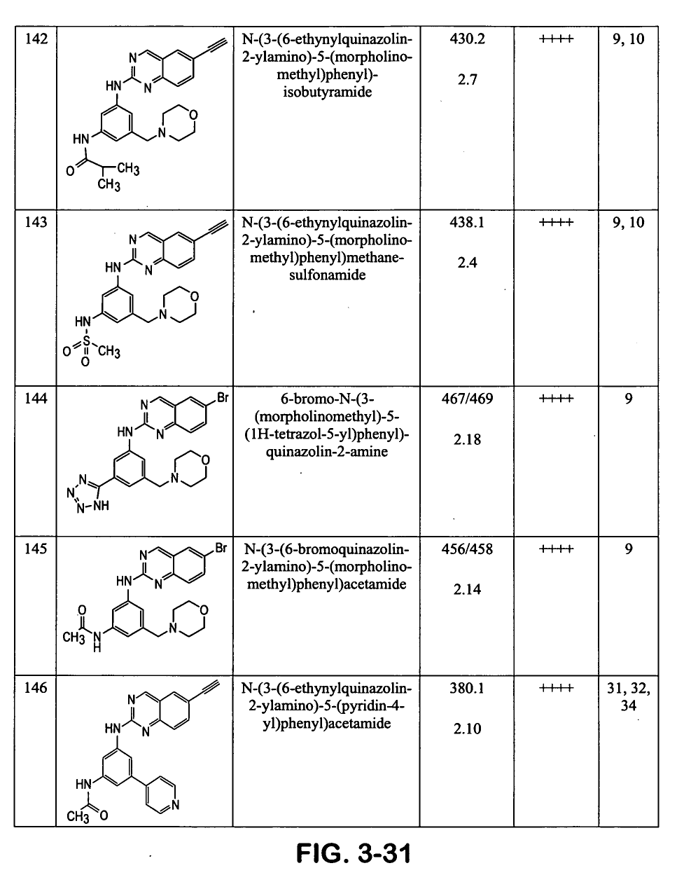

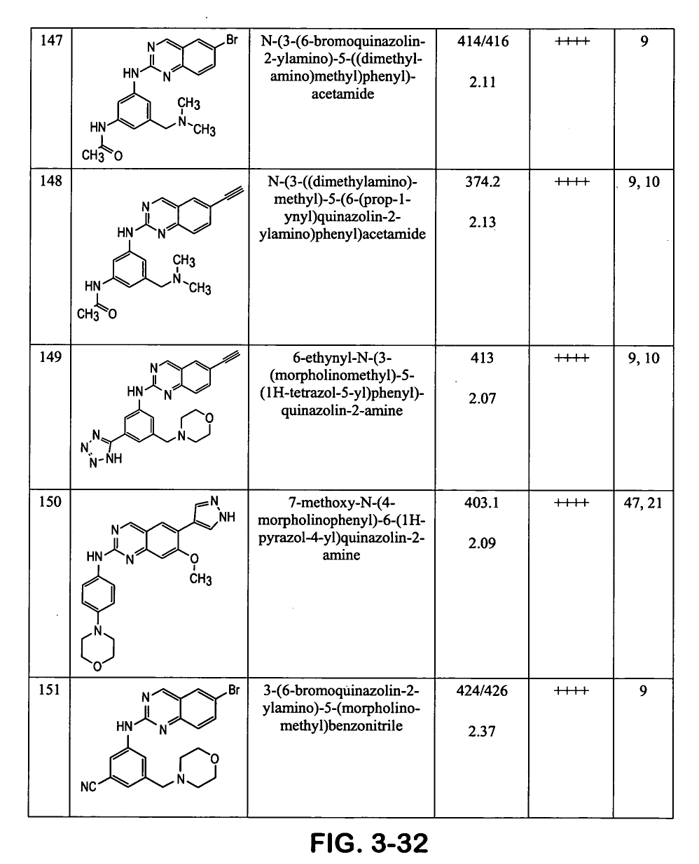

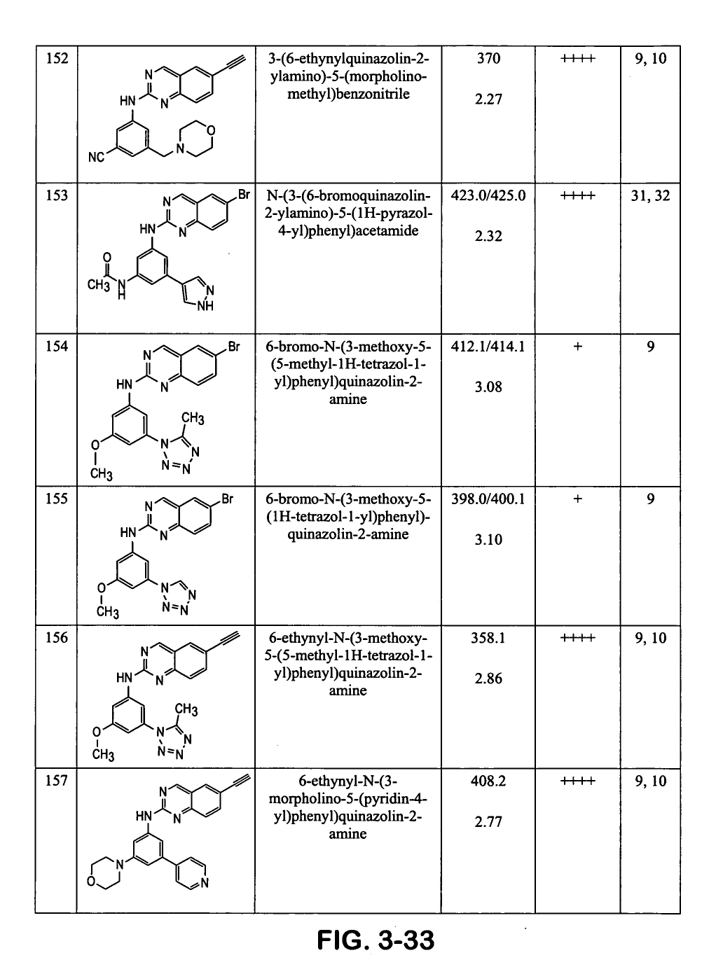

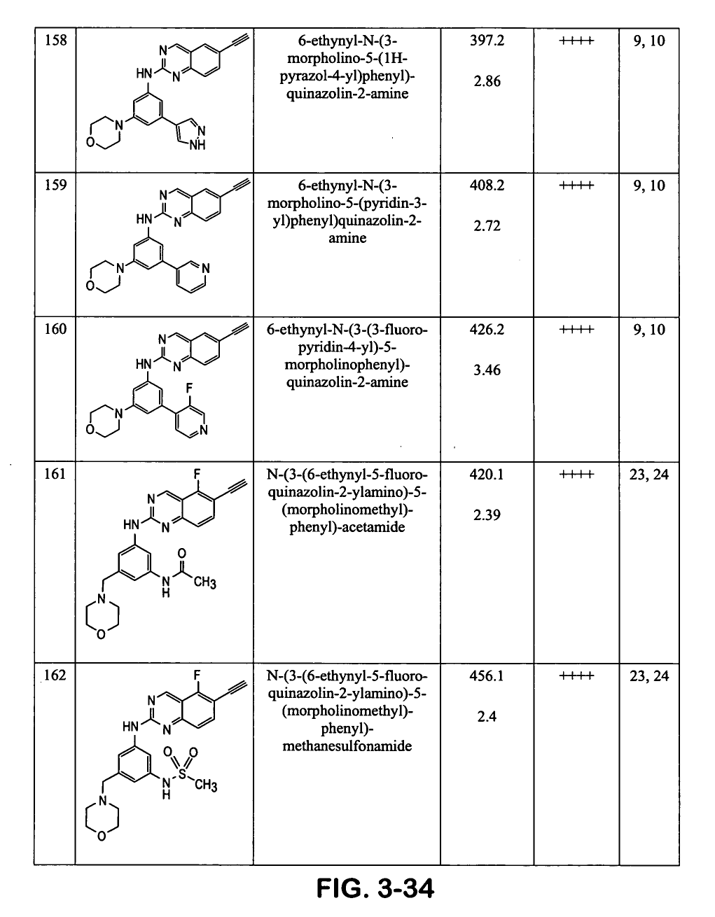

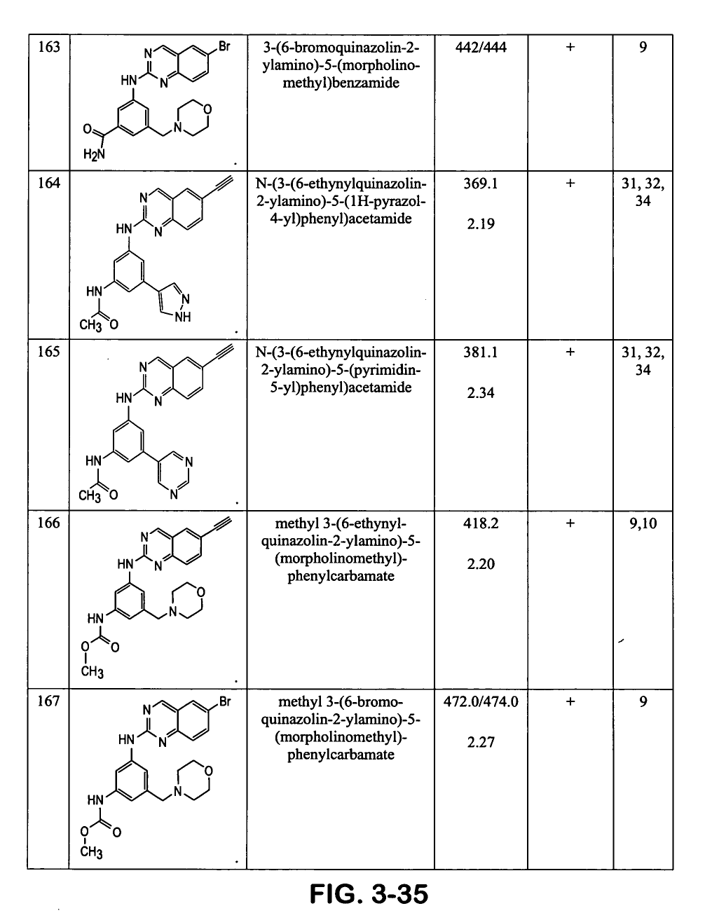

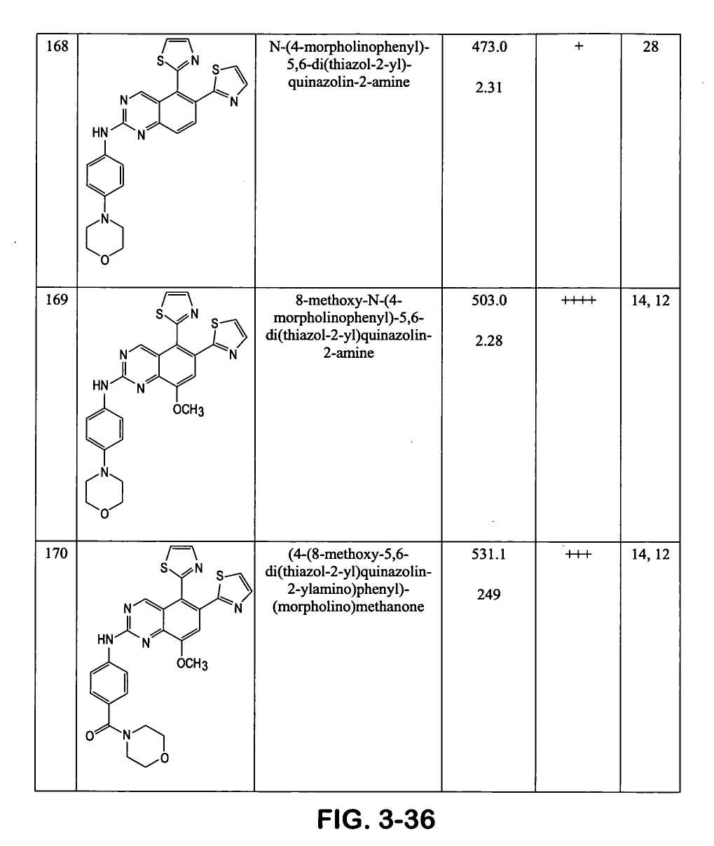

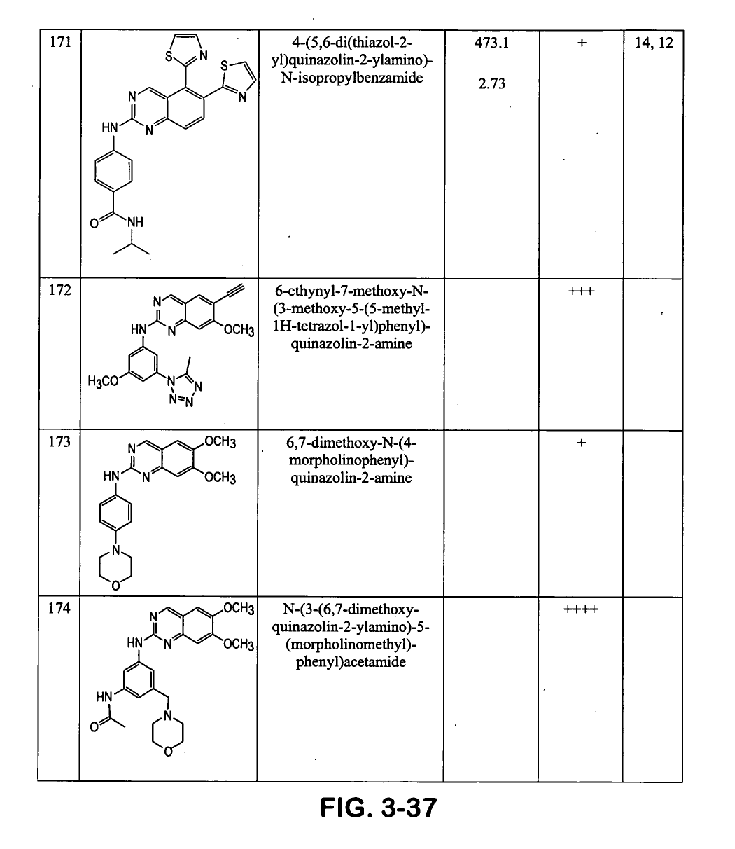

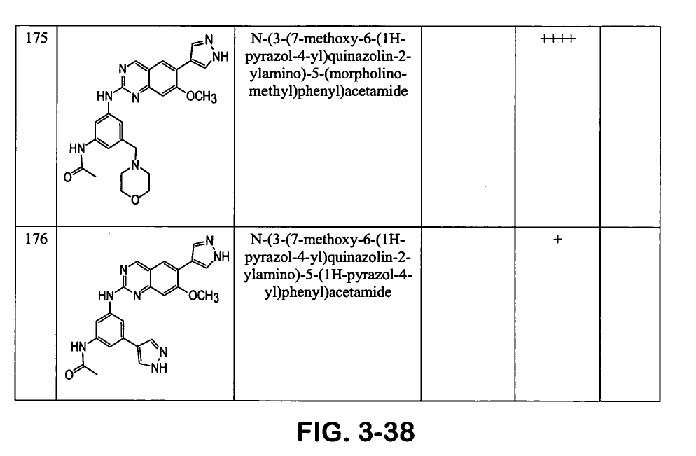

### DETAILED DESCRIPTION

In one aspect, the present invention provides compounds of Formula (Ia):

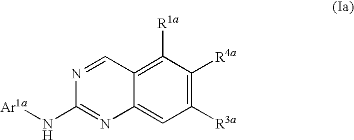

or pharmaceutically acceptable salt thereof, wherein:

R^{1a} is selected from H and halogen;

R^{3a} is selected from C₂₋₆ heterocycloalkyloxy and C₁₋₆ heteroaryl-C₁₋₄-alkoxy;

wherein said C₂₋₆ heterocycloalkyloxy and C₁₋₆ heteroaryl-C₁₋₄-alkoxy are each optionally substituted by a R^{w} group;

R^{4a} is selected from H, a thiazole ring, a pyrazole ring, a triazole ring, a tetrazole ring, a pyridine ring, C₃₋₆ cycloalkyl, cyano, halogen, C₂₋₆ alkynyl, C₁₋₆ heteroaryl-C₁₋₄-alkyl, C₁₋₆ heteroaryl-C₁₋₄-alkynyl, —C(═O)R^{a}, and —C(═O)NR^{b}R^{c}; wherein said thiazole ring, pyrazole ring, triazole ring, tetrazole ring, pyridine ring, C₁₋₆ heteroaryl-C₁₋₄-alkyl and C₁₋₆ heteroaryl-C₁₋₄-alkynyl are each optionally substituted by 1 or 2 independently selected R^{x″} groups;

Ar^{1a} is selected from phenyl optionally substituted at the meta position by one R^{y′} or, alternatively, at the para position by one R^{y″} group;

each R^{w} is independently selected from halogen, C₁₋₆ alkyl, amino, C₁₋₆ alkylamino, di-C₁₋₄-alkylamino, C₁₋₆ alkoxycarbonyl, and C₁₋₆ alkylcarbamyl; wherein said C₁₋₆ alkyl and C₁₋₆ alkylamino are each optionally substituted by a group selected from hydroxyl, C₁₋₆ alkoxy, amino, C₁₋₆ alkylamino, and di-Cᵢₛ-alkylamino;

each R^{x″} is independently selected from halogen, hydroxyl, C₁₋₆ alkyl, C₁₋₆ alkoxycarbonyl, and carbamyl; wherein said C₁₋₆ alkyl and C₁₋₆ alkoxycarbonyl are each optionally substituted by a group selected from hydroxyl, amino, C₁₋₄ alkylamino, and aminosulfonyl;

R^{y′} is selected from halogen, C₁₋₆ alkoxy, C₁₋₆ alkyl, carbamyl, aminosulfonyl, and C₁₋₆ alkylsulfonylamino; wherein said C₁₋₆ alkyl and C₁₋₆ alkoxy are each substituted by 1 or 2 groups independently selected from hydroxyl, amino, C₁₋₄ alkylamino, and aminosulfonyl;

R^{y″} is selected from C₁₋₆ haloalkyl;

R^{a} is selected from H, C₁₋₆ alkoxy and C₂₋₆ heterocycloalkyl;

R^{b} is selected from H and C₁₋₆ alkyl; and

R^{c} is selected from C₁₋₆ alkyl, C₃₋₆ cycloalkyl, C₁₋₆ heteroaryl, C₂₋₆ heterocycloalkyl, C₂₋₆ hetero cycloalkyl-C₁₋₄-alkyl, and C₁₋₆ heteroaryl-C₁₋₄-alkyl; wherein said C₁₋₆ alkyl is optionally substituted by a group selected from hydroxyl and C₁₋₄ alkoxy;

provided that:
(a) when R^{3a} is piperidin-4-yloxy or N-methylpiperidin-4-yloxy, then R⁴ is not selected from thiazol-2-yl, thiazol-4-yl, thiazol-5-yl, 5-methylthiazol-2-yl, 5-(hydroxymethyl)thiazol-2-yl, 5-(hydroxymethyl)thiazol-4-yl, pyridin-4-yl, pyridin-3-yl, 4-methylpyridin-3-yl, 5-chloropyridin-4-yl, 3-methylpyridin-2-yl, and 1-methylpyrazol-5-yl;
(b) when R^{4a} is ethynyl and Ar^{1a} is 3-fluorophenyl, then R³ is not selected from pyridin-3-ylmethoxy, 6-chloropyridin-3-ylmethoxy, pyridin-2-ylmethoxy, pyridin-4-ylmethoxy, 1-(pyridin-4-yl)ethoxy, 6-methoxypyridin-2-ylmethoxy, thiazol-5-ylmethoxy, pyrazin-2-ylmethoxy, 5-methylisooxazol-3-yl, azetidin-3-yloxy, N-methylazetidin-3-yloxy, N-isopropylazetidin-3-yloxy, pyrrolidin-3-yloxy, N-methyl, and pyrrolidin-3-yloxy;
(c) when R^{4a} is ethynyl, then Ar^{1a} is not selected from 3-(2-hydroxypropan-2-yl)phenyl, 3-(1-hydroxyethyl)phenyl, 3-aminosulfonylphenyl, phenyl, 3-(methylsulfonylamino)phenyl, 3-(N,N-dimethylaminomethyl)phenyl, 3-aminosulfonylphenyl, and 3-carbamylphenyl;
(d) when R^{4a} is thiazol-2-yl, then Ar^{1a} is not selected from 3-(methylaminosulfonyl)phenyl, 3-chlorophenyl, 3-carbamylphenyl, 3-fluorophenyl, 2-(hydroxypropan-2-yl)phenyl, 1-hydroxyethyl, 3-aminosulfonylphenyl, and phenyl;
(e) when R^{4a} is thiazol-2-yl and Ar^{1a} is 3-fluorophenyl, then R³ is not selected from pyridin-3-ylmethoxy, 6-chloropyridin-3-ylmethoxy, 2-chloropyridin-4-ylmethoxy, 5-methylisooxazol-3-yl, azetidin-3-yloxy, N-methylazetidin-3-yloxy, N-isopropylazetidin-3-yloxy, pyrrolidin-3-yloxy, N-methyl, pyrrolidin-3-yloxy, piperidin-4-yloxy, and N-methylpiperidin-4-yloxy;
(f) when R^{4a} is bromo, then Ar^{1a} is not selected from phenyl, 3-fluorophenyl, 2-(hydroxypropan-2-yl)phenyl, 1-hydroxyethyl, and 3-aminosulfonylphenyl;
(g) when R^{4a} is cyano, then R^{3a} is not selected from pyridin-4-ylmethoxy, pyridin-2-ylmethoxy, 6-methoxypyridin-2-ylmethoxy;
(h) when R^{4a} is cyclopropyl, then R^{3a} is not selected from azetidin-3-yloxy; and
(i) when R^{4a} is hydrogen, then Ar^{1a} is 3-(aminosulfonyl)phenyl.

In some embodiments:

R^{1a} is selected from H and halogen;

R^{3a} is selected from C₂₋₆ heterocycloalkyloxy and C₁₋₆ heteroaryl-C₁₋₄-alkoxy;

wherein said C₂₋₆ heterocycloalkyloxy and C₁₋₆ heteroaryl-C₁₋₄-alkoxy are each optionally substituted by a le group;

R^{4a} is selected from H, a thiazole ring, a pyrazole ring, a triazole ring, a tetrazole ring, a pyridine ring, C₃₋₆ cycloalkyl, cyano, bromo, C₂₋₆ alkynyl, C₁₋₆ heteroaryl-C₁₋₄-alkyl, C₁₋₆ heteroaryl-C₁₋₄-alkynyl, —C(═O)R^{a}, and —C(═O)NR^{b}R^{c}; wherein said thiazole ring; pyrazole ring, triazole ring, tetrazole ring, pyridine ring, C₁₋₆ heteroaryl-C₁₋₄-alkyl and C₁₋₆ heteroaryl-C₁₋₄-alkynyl are each optionally substituted by 1 or 2 independently selected R^{x″} groups;

Ar^{1a} is selected from phenyl optionally substituted at the meta position by one R^{y′} or, alternatively, at the para position by one R^{y″} group;

each R^{w} is independently selected from halogen, C₁₋₆ alkyl, amino, C₁₋₆ alkylamino, di-C₁₋₄-alkylamino, C₁₋₆ alkoxycarbonyl, and C₁₋₆ alkylcarbamyl; wherein said C₁₋₆ alkyl and C₁₋₆ alkylamino are each optionally substituted by a group selected from hydroxyl and amino;

each R^{x″} is independently selected from halogen, hydroxyl, C₁₋₆ alkyl, C₁₋₆ alkoxycarbonyl, and carbamyl; wherein said C₁₋₆ alkyl are each optionally substituted by a group selected from hydroxyl;

R^{x″} is selected from halogen, C₁₋₆ alkoxy, C₁₋₆ alkyl, carbamyl, aminosulfonyl, and C₁₋₆ alkylsulfonylamino; wherein said C₁₋₆ alkyl and C₁₋₆ alkoxy are each substituted by 1 or 2 groups independently selected from hydroxyl, amino, C₁₋₄ alkylamino, and aminosulfonyl;

R^{y′} is selected from C₁₋₆ haloalkyl;

R^{a} is selected from H, C₁₋₆ alkoxy and C₂₋₆ heterocycloalkyl;

R^{b} is selected from H and C₁₋₆ alkyl; and

R^{c} is selected from C₁₋₆ alkyl, C₁₋₆ heteroaryl, C₂₋₆ heterocycloalkyl, C₂₋₆ heterocycloalkyl-C₁₋₄-alkyl, and C₁₋₆ heteroaryl-C₁₋₄-alkyl; wherein said C₁₋₆ alkyl is optionally substituted by a group selected from hydroxyl and C₁₋₄ alkoxy.

In some embodiments, Ar^{1a} is selected from 3-fluorophenyl, 3-methylaminosulfonyl, 3-carbamylphenyl, 3-chlorophenyl, 4-difluoromethylphenyl, -(2-amino-n-propyl)methylphenyl, 3-(1-amino-3-hydroxy-n-propyl)phenyl, 3-(2-aminoethyl)phenyl, 3-(aminomethyl)phenyl, 3-(3-amino-1-hydroxy)phenyl, 3-(2-hydroxyethyl)phenyl, 3-(hydroxymethyl)phenyl, 3-methylaminosulfonyl, 3-(aminosulfonylmethyl)phenyl, 3-carbamylphenyl and 3-chlorophenyl.

In some embodiments, Ar^{1a} is 3-halophenyl, wherein said halo is not fluoro. In some embodiments, Ar^{1a} is 3-fluorophenyl and R^{1a} is halogen. In some embodiments, Ar^{1a} is not phenyl.

In some embodiments, R^{3a} is selected from piperidin-4-yloxy, N-methylpiperidin-4-yloxy, N-(tert-butoxycarbonyl)piperidin-4-yloxy, 3-fluoropiperidin-4-yloxy, 2-(hydroxymethyl)piperidin-4-yloxy, N-(tert-butoxycarbonyl)piperidin-4-yl, 2-(N-methylcarbamyl)piperidin-4-yloxy, 6-methoxypyridin-3-ylmethoxy, 6-(N-methylamino)pyridin-3-ylmethoxy, 6-(N,N-dimethylamino)pyridin-3-ylmethoxy, 6-(N-(2-hydroxyethyl)amino)pyridin-3-ylmethoxy, 6-(N-(2-methoxyethyl)amino)pyridin-3-ylmethoxy, 6-(N-(2-aminoethyl)amino)pyridin-3-ylmethoxy, and pyridin-3-ylmethoxy.

In some embodiments, R^{3a} is C₁₋₆ heteroaryl-C₁₋₄-alkoxy; which is optionally substituted by a R^{w} group. In some embodiments, R^{3a} is C₂₋₆ heterocycloalkyloxy which is optionally substituted by a R^{w} group. In some embodiments, R^{3a} is C₂₋₆ heterocycloalkyloxy which is substituted by a R^{w} group, wherein R^{w} is not methyl or isopropyl. In some embodiments, R^{3a} is C₂₋₆ heterocycloalkyloxy which is substituted by a R^{w} group, wherein R^{w} is not methyl. In some embodiments, R^{3a} is C₂₋₆ heterocycloalkyloxy which is substituted by a R^{w} group, wherein R^{w} is not methyl, ethyl or isopropyl. In some embodiments, R^{3a} is C₁₋₆ heteroaryl-C₁₋₄-alkoxy; which is substituted by a R^{w} group. In some embodiments, R^{3a} is C₁₋₆ heteroaryl-C₁₋₄-alkoxy; which is substituted by a R^{w} group, which is not a methyl group. In some embodiments, R^{3a} is C₁₋₆ heteroaryl-C₁₋₄-alkoxy; which is substituted by a R^{w} group, which is not a methyl, ethyl, or isopropyl group. In some embodiments, R^{3a} is C₁₋₆ heteroaryl-C₁₋₄-alkoxy; wherein said C₁₋₄-alkoxy is not methoxy. In some embodiments, R^{3a} is C₁₋₆ heteroaryl-C₁₋₄-alkoxy; wherein said heteroaryl group is a pyridine group which is substituted by a R^{w} group. In some embodiments, R^{3a} is C₁₋₆ heteroaryl-C₁₋₄-alkoxy; wherein said heteroaryl group is a pyridine group which is substituted by a R^{w} group, which is not selected from C₁₋₆ alkyl.

In some embodiments, R^{4a} is selected from ethynyl, bromo, cyano, cyclopropyl, thiazol-2-yl, pyridin-3-yl, 4-(hydroxymethyl)thiazol-2-yl, 1,2,3-triazol-5-yl, tetrazol-5-yl, pyrazol-2-yl, 5-methylpyrazol-2-yl, 2-(pyridin-2-yl)ethynyl, 2-(pyridin-2-yl)ethyl, 2-(pyridin-3-yl)ethynyl, 2-(pyridin-3-yl)ethyl, 4-(methoxycarbonyl)thiazol-2-yl, 4-carbamylthiazol-2-yl, —C(═O)R^{a}, and —C(═O)NR^{b}R^{c}.

In some embodiments, R^{4a} is ethynyl. In some embodiments, R^{4a} is bromo. In some embodiments, R^{4a} is cyano. In some embodiments, R^{4a} is cyclopropyl. In some embodiments, R^{4a} is thiazol-2-yl. In some embodiments, R^{4a} is pyridin-3-yl. In some embodiments, R^{4a} is 4-(hydroxymethyl)thiazol-2-yl. In some embodiments, R^{4a} is 1,2,3-triazol-5-yl. In some embodiments, R^{4a} is tetrazol-5-yl. In some embodiments, R^{4a} is pyrazol-2-yl. In some embodiments, R^{4a} is 5-methylpyrazol-2-yl. In some embodiments, R^{4a} is (pyridin-2-yl)ethynyl. In some embodiments, R^{4a} is 2-(pyridin-2-yl)ethyl. In some embodiments, R^{4a} is 2-(pyridin-3-yl)ethynyl. In some embodiments, R^{4a} is 2-(pyridin-3-yl)ethyl. In some embodiments, R^{4a} is 4-(methoxycarbonyl)thiazol-2-yl. In some embodiments, R^{4a} is 4-carbamylthiazol-2-yl. In some embodiments, R^{4a} is —C(═O)R^{a} or —C(═O)NR^{b}R^{c}, wherein said R^{a} is not carboxy. In some embodiments, R^{4a} is —C(═O)R^{a} or —C(═O)NR^{b}R^{c}, wherein said R^{a} is not carboxy or C₁₋₆ alkoxy. In some embodiments, R^{4a} is —C(═O)R^{a} or —C(═O)NR^{b}R^{c}. In some embodiments, R^{4a} is —C(═O)R^{a}. In some embodiments, R^{4a} is —C(═O)NR^{b}R^{c}. In some embodiments, R^{4a} is C₂₋₆ alkoxy. In some embodiments, R^{4a} is halogen. In some embodiments, R^{4a} is In some embodiments, R^{4a} is C₃₋₆ cycloalkyl, which is optionally substituted by 1 or 2 independently selected R^{x″} groups. In some embodiments, R^{4a} is a thiazole ring, which is optionally substituted by 1 or 2 independently selected R^{x″} groups. In some embodiments, R^{4a} is a pyridine ring, which is optionally substituted by 1 or 2 independently selected R^{x″} groups. In some embodiments, R^{4a} is 1,2,3-triazol-5-yl, which is optionally substituted by 1 or 2 independently selected R^{x″} groups. In some embodiments, R^{4a} is a tetrazole ring, which is optionally substituted by 1 or 2 independently selected R^{x″} groups. In some embodiments, R^{4a} is a pyrazole ring, which is optionally substituted by 1 or 2 independently selected R^{x″} groups. In some embodiments, R^{4a} is (a pyridine ring)-alkynyl, wherein said pyridine ring is optionally substituted by 1 or 2 independently selected R^{x″} groups. In some embodiments, R^{4a} is 2-(pyridin-2-yl)ethyl. In some embodiments, R^{4a} is (a pyridine ring)-alkyl, wherein said pyridine ring is optionally substituted by 1 or 2 independently selected R^{x″} groups. In some embodiments, R^{1a} is selected from H and chloro.

In some embodiments, R^{1a} is selected from H. In some embodiments, R^{1a} is selected from chloro. In some embodiments, R^{1a} is halogen.

In some embodiments:
R^{1a} is selected from H;
Ar^{1a} is 3-fluorophenyl;
R^{3a} is selected from C₂₋₆ heterocycloalkyloxy which is substituted by a 117 group; and
R^{4a} is selected from a thiazole ring and a pyridine ring, each of which is optionally substituted by 1 or 2 independently selected R^{x″} groups.

In some embodiments:
R^{1a} is selected from H;
Ar^{1a} is 3-fluorophenyl;
R^{3a} is selected from 3-fluoropiperidin-4-yloxy, 2-(hydroxymethyl)piperidin-4-yloxy, N-(tert-butoxycarbonyl)piperidin-4-yl, and 2-(N-methylcarbamyl)piperidin-4-yloxy; and
R^{4a} is selected from thiazol-2-yl and pyridin-3-yl.

In some embodiments:
R^{1a} is selected from H;
Ar^{1a} is 3-fluorophenyl;
R^{3a} is selected from C₁₋₆ heteroaryl-C₁₋₄-alkoxy, which is substituted by a R^{w} group;
R^{4a} is C₂₋₆ alkynyl, which is optionally substituted by 1 or 2 independently selected R^{x″} groups.

In some embodiments:
R^{1a} is selected from H;
Ar^{1a} is 3-fluorophenyl;
R^{3a} is selected from 6-methoxypyridin-3-ylmethoxy, 6-(N-methylamino)pyridin-3-ylmethoxy, 6-(N,N-dimethylamino)pyridin-3-ylmethoxy, 6-(N-(2-hydroxyethyl)amino)pyridin-3-ylmethoxy, 6-(N-(2-methoxyethyl)amino)pyridin-3-ylmethoxy, and 6-(N-(2-aminoethyl)amino)pyridin-3-ylmethoxy; and
R^{4a} is ethynyl.

In some embodiments:

R^{1a} is selected from H;
R^{3a} is C₁₋₆ heteroaryl-C₁₋₄-alkoxy; which is optionally substituted by a group;
R^{4a} is selected from halogen and C₂₋₆ alkynyl, each of which is optionally substituted by 1 or 2 independently selected R^{x″} groups;
Ar^{1a} is selected from phenyl substituted at the meta position by one R^{y′}; and
R^{y′} is selected from C₁₋₆ alkyl which is substituted by 1 or 2 groups independently selected from hydroxyl, amino, C₁₋₄ alkylamino, and aminosulfonyl.

In some embodiments:
R^{1a} is selected from H;
Ar^{1a} is selected from 3-(2-amino-n-propyl)methylphenyl, 3-(1-amino-3-hydroxy-n-propyl)phenyl, 3-(2-aminoethyl)phenyl, 3-(aminomethyl)phenyl, 3-(3-amino-1-hydroxy)phenyl, 3-(2-hydroxyethyl)phenyl, and 3-(hydroxymethyl)phenyl;
R^{3a} is selected from pyridin-3-ylmethoxy; and
R^{4a} is selected from ethynyl and bromo.

In some embodiments:
R^{1a} is selected from H;
R^{3a} is selected from C₁₋₆ heteroaryl-C₁₋₄-alkoxy; which is optionally substituted by a R^{w} group;
R^{4a} is selected from a thiazole ring, which is optionally substituted by 1 or 2 independently selected groups;
Ar^{1a} is selected from phenyl optionally substituted at the meta position by one R^{y′}; and
R^{y′} is selected from halogen, C₁₋₆ alkoxy, carbamyl, aminosulfonyl, and C₁₋₆ alkylsulfonylamino; wherein said C₁₋₆ alkyl and C₁₋₆ alkoxy are each substituted by 1 or 2 groups independently selected from hydroxyl, amino, C₁₋₄ alkylamino, and aminosulfonyl.

In some embodiments:
R^{1a} is selected from H;
A^{1a} is selected from 3-methylaminosulfonyl, 3-(aminosulfonylmethyl)phenyl, and phenyl;
R^{3a} is selected from piperidin-4-yloxy; and
R^{4a} is thiazol-2-yl and 4-(hydroxymethyl)thiazol-2-yl.

In some embodiments:
R^{1a} is selected from H;
Ar^{1a} is 3-fluorophenyl;
R^{3a} is selected from C₂₋₆ heterocycloalkyloxy; which is optionally substituted by a R^{w} group; and
R^{4a} is selected from a thiazole ring, a pyrazole ring, a triazole ring, a tetrazole ring, a pyridine ring, C₃₋₆ cycloalkyl, and cyano; wherein said pyrazole ring, triazole ring, tetrazole ring, pyridine ring, C₁₋₆ heteroaryl-C₁₋₄-alkyl and C₁₋₆ heteroaryl-C₁₋₄-alkynyl are each optionally substituted by 1 or 2 independently selected R^{x″} groups; and wherein said thiazole ring is substituted by 1 or 2 independently selected R^{x″} groups.

In some embodiments:
R^{1a} is selected from H;
Ar^{1a} is 3-fluorophenyl;
R^{3a} is selected from piperidin-4-yloxy; and
R^{4a} is 1,2,3-triazol-5-yl, cyclopropyl, cyano, tetrazol-5-yl, pyrazol-2-yl, 5-methylpyrazol-2-yl, 2-(pyridin-2-yl)ethynyl, 2-(pyridin-2-yl)ethyl, 2-(pyridin-3-yl)ethynyl, 2-(pyridin-3-yl)ethyl, 4-(methoxycarbonyl)thiazol-2-yl, and 4-carbamylthiazol-2-yl.

In some embodiments:
R^{1a} is selected from H;
Ar^{1a} is 3-fluorophenyl
R^{3a} is selected from C₂₋₆ heterocycloalkyloxy and C₁₋₆ heteroaryl-C₁₋₄-alkoxy; wherein said C₂₋₆ heterocycloalkyloxy and C₁₋₆ heteroaryl-C₁₋₄-alkoxy are each optionally substituted by a R^{w} group;
R^{4a} is selected —C(═O)R^{a} and —C(═O)NR^{b}R^{c};
R^{a} is selected from H, C₁₋₆ alkoxy and C₂₋₆ heterocycloalkyl;

R^{b} is selected from H and C₁₋₆ alkyl; and

R^{c} is selected from C₁₋₆ alkyl, C₁₋₆ heteroaryl, C₂₋₆ heterocycloalkyl, C₂₋₆ heterocycloalkyl-C₁₋₄-alkyl, and C₁₋₆ heteroaryl-C₁₋₄-alkyl; wherein said C₁₋₆ alkyl is optionally substituted by a group selected from hydroxyl and C₁₋₄ alkoxy.

In some embodiments:
R^{1a} is selected from H;
Ar^{1a} is 3-fluorophenyl
R^{3a} is selected from piperidin-4-yloxy and N-(tert-butoxycarbonyl)piperidin-4-yloxy;
R^{4a} is selected from —C(═O)R^{a}, and —C(═O)NR^{b}R^{c};
R^{a} is selected from H, methoxy and morpholin-4-yl;
R^{b} is selected from H and methyl; and
R^{c} is selected from methyl, 2-hydroxyethyl, 2-methoxyethyl, tetrahydro-2H-pyran, tetrahydropyran-2H-methyl, 2-oxopyrrolidinylethyl, and pyridin-3-ylmethyl.

In some embodiments:
R^{1a} is selected from H;
Ar^{1a} is 3-carbamylphenyl and 3-chlorophenyl;
R^{3a} is selected from C₁₋₆ heteroaryl-C₁₋₄-alkoxy; which is optionally substituted by a R^{w} group; and
R^{4a} is selected from C₂₋₆ alkynyl and bromo.

In some embodiments:
R^{1a} is selected from H;
Ar^{1a} is 3-carbamylphenyl and 3-chlorophenyl;
R^{3a} is selected from pyridin-4-ylmethoxy; and
R^{4a} is selected from ethynyl and bromo.

In some aspects, the present invention provides compounds of Formula (IIa):

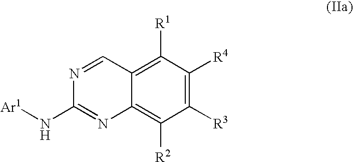

or pharmaceutically acceptable salt thereof, wherein:
R¹ is selected from H and halogen
R² is selected from H, halogen, and C₁₋₆ alkoxy;
R³ is selected from H, —OCH₂-phenyl, —O—CH₂-Het, —OCH₂—CH₂-Het, and —O—Hy; wherein Het is 6-membered heteroaryl which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and phenyl is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl;
R⁴ is selected from cyano, halogen, C₁₋₆ alkyl, C₂₋₆ alkynyl, C₃₋₆ cycloalkyl, a thiazole ring, a pyrazole ring, and a pyridine ring; each of which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups;
Ar¹ is a moiety of Group (A), (B), or (C):

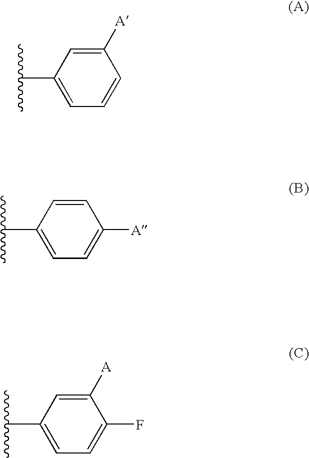

A is a pyrazole ring; which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino;
A′ is selected from -L²-Ar² and -L¹-Cy¹;
A″ is selected from -L^{2a}-Ar^{2a} and —Cy^{1a};
L¹ is selected from a bond, —O—, —C(═O)—, —CH₂C(═O)—, and —CH₂—; wherein the left end of the linker is attached to the phenyl ring and the right end of the linker is attached to Cy¹;
Cy¹ is selected from a morpholine ring, a tetrahydro-2H-pyran ring, a pyrrolidine ring, a 2-oxopyrrolidine ring, and a piperidine ring, each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
Cy^{1a} is selected from a morpholine ring, a 2-oxopyrrolidine ring and a piperidine ring; each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
L² and L^{2a} are each independently selected from a bond, —O—, and —CH₂—; and
Ar² is selected from a pyrazole ring, an oxazole ring, an imidazole ring, a triazole ring, a thiadiazole ring, a pyridine ring, and a pyrimidine ring; each of which is optionally substituted with 1, 2, or 3 groups independently selected from hydroxyl, C₁₋₆ alkyl, C₁₋₄ alkoxy, amino, C₁₋₄ alkylamino, and di-C₁₋₄ alkylamino;
Ar^{2a} is selected from a pyrazole ring, a triazole ring, a pyridine ring, and a pyrimidine ring; each of which is optionally substituted with 1 or 2 groups independently selected from hydroxyl, C₁₋₆ alkyl, C₁₋₄ alkoxy, amino, C₁₋₄ alkylamino, and di-C₁₋₄ alkylamino;

provided that:
(i) when R¹ and R² are each H, R⁴ is thiazol-2-yl, R³ is optionally substituted piperidin-4-yloxy, and Ar¹ is a moiety of Group (A), then A′ is not selected from 1,2,4-triazol-1-ylmethyl, 1-methylpyrazol-3-yl, 2-oxopyrrolidinyl, oxazol-5-yl, pyrazol-1-yl, 1-methyl-1,2,4-triazol-2-yl, morpholin-4-ylcarbonylmethyl, imidazol-2-yl, 2-methylthiazol-4-yl, 1,3,5-trimethylpyrazol-4-yl, pyrimidin-5-yl, 1,2,4-triazol-1-yl, 4,5-dimethyloxazol-2-yl, pyrimidin-5-yl, 2-methoxypyrimidin-5-yl, 6-methoxypyridin-3-yl, and pyridin-3-yl;
(ii) when R¹ and R² are each H, Ar¹ is a moiety of Group (A), A′ is selected from 1-methylpyrazol-3-yl, and R³ is selected from pyridin-3-ylmethoxy and pyrazin-2-ylmethoxy, then R⁴ is not thiazol-2-yl;
(iii) when R¹ and R² are each H, R⁴ is thiazol-2-yl, R³ is optionally substituted piperidin-4-yloxy, and Ar¹ is a moiety of Group (B), then A″ is not selected from morpholin-4-yl, morpholin-4-ylcarbonylmethyl, pyrimidin-5-yl, and pyrazol-1-ylmethyl;
(iv) when R¹ and R² are each H, R⁴ is bromo, R³ is pyridin-3-ylmethyl, and Ar¹ is a moiety of Group (A), then A′ is not piperidin-4-yl;
(v) when R¹ and R³ are each H, R⁴ is bromo or ethynyl, R² is isopropoxy, and Ar¹ is a moiety of Group (A), then A′ is not selected from 1-methyl-1,2,4-triazol-2-ylmethyl and morpholin-4-ylcarbonylmethyl;
(vi) when R¹ and R² are each H, R⁴ is cyclopropyl, bromo or ethynyl, R³ is chloro, and Ar¹ is a moiety of Group (A), then A′ is not 1,2,4-triazol-1-ylmethyl;
(vii) when R¹ and R² are each H, R⁴ is ethynyl, R³ is chloro, and Ar¹ is a moiety of Group (A), then A′ is not imidazol-2-yl, oxazol-5-yl, pyrazol-1-yl, and morpholin-4-ylmethyl;
(viii) when R¹ and R³ are each H, R⁴ is ethynyl, R¹ is chloro, and Ar¹ is a moiety of Group (A), then A′ is not selected from pyrazol-1-yl, oxazol-5-yl, imidazol-2-yl, morpholin-4-ylcarbonylmethyl, and 2-oxopyrrolidinyl;
(ix) when R¹ and R² are each H, R⁴ is ethynyl, R³ is H, and Ar¹ is a moiety of Group (A), then A′ is not selected from 1,2,4-triazol-1-ylmethyl, pyrazol-1-yl, oxazol-5-yl, imidazol-2-yl, 5-trifluoromethylimidazol-2-yl, imidazol-1-ylmethyl, 1,2,4-triazol-1yl-methyl, N-(ethoxycarbonyl)piperidin-4-yl, N-(methoxycarbonyl)piperidin-4-yl, N-(acetyl)piperidin-4-yl, N-methylpiperidin-4-yl, piperidin-4-yl, and 2-oxopyrrolidinyl;
(x) when R¹ and R² are each H, R⁴ is ethynyl, Ar¹ is a moiety of Group (A), and A′ is 1-methylpyrazol-3-yl, then R³ is not selected from pyridin-3-ylmethoxy, 2-pyridin-3-ylethoxy, thiazol-5-ylmethoxy, and pyrazin-2-ylmethoxy;
(xi) when R¹ and R² are each H, R⁴ is ethynyl, Ar¹ is a moiety of Group (A), and A′ is morpholin-4-ylmethyl, then R³ is not selected from pyridin-3-ylmethoxy and pyrazin-2-ylmethoxy;
(xii) when R¹ and R² are each H, R⁴ is ethynyl, Ar¹ is a moiety of Group (A), and A′ is oxazol-5-yl, then R³ is not selected from pyridin-3-ylmethoxy and pyrazin-2-ylmethoxy;
(xiii) when R¹ and R² are each H, R⁴ is ethynyl, R³ is H, and Ar¹ is a moiety of Group (B), then A″ is not selected from morpholin-4-ylmethyl and 2-oxopyrrolidinyl;
(xiv) when R¹ and R² are each H, R⁴ is ethynyl, R³ is chloro, and Ar¹ is a moiety of Group (B), then A″ is not selected from 2-oxopyrrolidinyl and morpholin-4-ylmethyl;
(xv) when R¹ and R² are each H, R³ is pyrazin-2-ylmethoxy, R⁴ is pyrazol-4-yl, and Ar¹ is a moiety of Group (A), then A′ is not 1-methylpyrazol-3-yl;
(xvi) when R¹ and R² are each H, R³ is piperidin-4-yloxy or N-methylpiperidin-4-yloxy, R⁴ is pyrazol-4-yl, and Ar¹ is a moiety of Group (B), then A″ is not morpholin-4-yl;
(xvii) when Ar¹ is 3-morpholin-4-ylphenyl, then R³ is not H;
(xviii) when Ar¹ is 4-morpholin-4-ylphenyl, then R⁴ is not selected from bromo, cyano, ethynyl, thiazol-2-yl, and 1H-pyrazol-4-yl;
(xix) when R^{1′} is H, R^{2′} is H, R^{3′} is piperidin-4-yloxy, and R^{4′} is 6-methoxypyridin-3-yl, and Ar¹ is a moiety of Group (A), then A′ is not 6-methoxypyridin-3-yl; and
(xx) when R^{1′} is H, R^{2′} is H, R^{3′} is piperidin-4-yloxy, and R^{4′} is 5-methoxypyridin-3-yl, and Ar¹ is a moiety of Group (B), then A″ is not 5-methoxypyridin-3-yl.

In some embodiments, R³ is H. In some embodiments, R³ is —OCH₂-phenyl. In some embodiments, R³ is —O—CH₂-Het. In some embodiments, R³ is —OCH₂—CH₂-Het. In some embodiments, R³ is —O—Hy.

In some embodiments, Het is 6-membered heteroaryl which is substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl. In some embodiments, Hy is 6-membered heterocycloalkyl, which is substituted by 1 or 2 groups independently selected from hydroxyl, C₁₋₆ alkyl, C₁₋₄ alkoxy, amino, C₁₋₄ alkylamino, alkylamino, and C₁₋₆ alkylcarbamyl. In some embodiments, Het is 6-membered heteroaryl which is substituted by 1 or 2 groups independently selected from hydroxyl, C₁₋₄ alkoxy, amino, C₁₋₄ alkylamino, di-C₁₋₄ alkylamino, and C₁₋₆ alkylcarbamyl. In some embodiments, Hy is 6-membered heterocycloalkyl, which is substituted by 1 or 2 groups independently selected from hydroxyl, C₁₋₄ alkoxy, amino, C₁₋₄ alkylamino, di-C₁₋₄ alkylamino, and C₁₋₆ alkylcarbamyl.

In some embodiments, R⁴ is cyano. In some embodiments, R⁴ is halogen. In some embodiments, R⁴ is C₁₋₆ alkyl, which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups. In some embodiments, R⁴ is C₂₋₆ alkynyl, which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups. In some embodiments, R⁴ is C₃₋₆ cycloalkyl. In some embodiments, R⁴ is a thiazole ring, which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups. In some embodiments, R⁴ is a pyrazole ring, which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups. In some embodiments, R⁴ is a pyridine ring; which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups. In some embodiments, R⁴ is selected from 1H-pyrazol-4-yl, 1-methyl-1H-pyrazol-4-yl, 1H-pyrazol-3-yl, 1-methyl-1H-pyrazol-4-yl, and 1-methyl-1H-pyrazol-5-yl.

In some embodiments, R⁴ is not thiazol-2-yl. In some embodiments, R⁴ is not ethynyl. In some embodiments, R⁴ is not bromo. In some embodiments, R⁴ is chloro. In some embodiments of each of the preceding embodiments, at least one of R¹, R², or R³ is not H.

In some embodiments, R¹ is H. In some embodiments, R¹ is halogen. In some embodiments, R¹ is chloro.

In some embodiments, R² is H. In some embodiments, R² is halogen. In some embodiments, R² is chloro. In some embodiments, R² is fluoro. In some embodiments,

R² is bromo. In some embodiments, R² is C₁₋₆ alkoxy. In some embodiments, R² is methoxy.

In some embodiments, at least one of R¹, R², or R³ is not H.

In some embodiments, L¹ is a bond. In some embodiments, L¹ is —O—. In some embodiments, L¹ is —C(═O)—. In some embodiments, L¹ is —CH₂C(═O)—. In some embodiments, L¹ is —CH₂—.

In some embodiments, L² is a bond. In some embodiments, L² is —O—. In some embodiments, L² is —CH₂—. In some embodiments, L^{2a} is a bond. In some embodiments, L^{2a} is —O—. In some embodiments, L^{2a} is —CH₂—.

In some embodiments, Ar² is a pyridine ring, which is optionally substituted by 1 or 2 groups independently selected from C₁₋₆ alkyl and C₁₋₆ alkoxy. In some embodiments, Ar² is a pyrimidine ring, which is optionally substituted by 1 or 2 groups independently selected from C₁₋₆ alkyl and C₁₋₆ alkoxy. In some embodiments, Ar² is 1H-pyrazole ring, which is optionally substituted by 1 or 2 groups independently selected from C₁₋₆ alkyl and C₁₋₆ alkoxy. In some embodiments, Ar² is an oxazole ring, which is optionally substituted by 1 or 2 groups independently selected from C₁₋₆ alkyl and C₁₋₆ alkoxy. In some embodiments, Ar² is a 1,2,4-triazole ring, which is optionally substituted by 1 or 2 groups independently selected from C₁₋₆ alkyl and C₁₋₆ alkoxy. In some embodiments, Ar² is a thiadiazole ring; which is optionally substituted by 1 or 2 groups independently selected from C₁₋₆ alkyl and C₁₋₆ alkoxy. In some embodiments, Ar² is selected from 5-methoxypyridin-3-yl, pyrimidin-5-yl, 1H-pyrazol-3-yl, 1H-pyrazol-5-yl, oxazol-5-yl, thiadiazol-4-yl, 1-methyl-1H-pyrazol-3-yl, 1,2,4-triazol-1-yl, 1H-pyrazol-1-yl, and 1H-imidazol-1-yl.

In some embodiments, A′ is selected from 1,2,4-triazol-1-ylmethyl, 1H-pyrazol-1-ylmethyl, and 1H-imidazol-1-ylmethyl.

In some embodiments, Ar^{1a} is selected from 1-methyl-1H-pyrazol-4-yl, 6-methyl-1H-pyrazol-3-yl, 2-methylpyrimidin-5-yl, pyridin-4-yl, pyridin-3-yl, 6-methoxypyridin-3-yl, 2-methoxypyridin-3-yl, 1,2,4-triazol-5-yl and pyrimidin-2-yl.

In some embodiments, Cy¹ is a morpholine ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Cy¹ is a tetrahydro-2H-pyran ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Cy¹ is a pyrrolidine ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl. In some embodiments, Cy¹ is a 2-oxopyrrolidine ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl. In some embodiments, Cy¹ is a piperidine ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Cy¹ is a morpholine ring, which is substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl. In some embodiments, Cy¹ is a tetrahydro-2H-pyran ring, which is substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl. In some embodiments, Cy¹ is a pyrrolidine ring, which is substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl. In some embodiments, Cy¹ is a 2-oxopyrrolidine ring, which is substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl. In some embodiments, Cy¹ is a piperidine ring, which is substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Cy^{1a} is a morpholine ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Cy^{1a} is a 2-oxopyrrolidine ring, which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Cy^{1a} is a piperidine ring; which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl.

In some embodiments, Ar¹ is a moiety of Group (A):

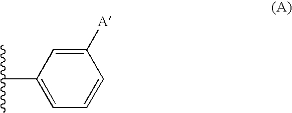

In some embodiments, A′ is -L²-Ar². In some embodiments, A′ is -L¹-Cy¹.

In some embodiments:
A′ is selected from A′ is -L²-Ar²;
L² is a bond;
Ar² is selected from a pyridine ring, a pyrimidine ring, 1H-pyrazole ring, an oxazole ring, a 1,2,4-triazole ring, and a thiadiazole ring; each of which is optionally substituted by 1 or 2 groups independently selected from C₁₋₆ alkyl and C₁₋₆ alkoxy;
R¹ is H;
R² is H;
R³ is selected from —O-phenyl, —O—CH₂-Het, —O—CH₂—CH₂-Het, and —O—Hy;
   wherein Het is a pyrazine ring, a pyridine ring, a pyrimidine ring, or a pyridazine ring; which is optionally substituted by a C₁₋₆ alkyl group; Hy is piperidine ring, which is optionally substituted by a C₁₋₆ alkyl group; and phenyl is optionally substituted by a C₁₋₆ alkylcarbamyl group;
R⁴ is selected from C₂₋₆ alkynyl, thiazol-2-yl, 1H-pyrazol-4-yl, 1-methyl-1H-pyrazol-4-yl, and 1H-pyrazol-3-yl.

In some embodiments:
A′ is selected from A′ is -L²-Ar²;
L² is a bond;
Ar² is selected from 5-methoxypyridin-3-yl, pyrimidin-5-yl, 1H-pyrazol-3-yl, 1H-pyrazol-5-yl, oxazol-5-yl, 1,2,4-triazol-5-yl, thiadiazol-4-yl, and 1-methyl-1H-pyrazol-3-yl;
R¹ is H;
R² is H;
R³ is selected from piperidin-4-yloxy, N-methylpiperidin-4-yloxy, 3-(N-methylcarbamyl)benzyloxy, pyrazin-2-ylmethoxy, pyridin-3-ylmethoxy, pyridimidin-5-ylmethoxy, pyridazin-3-ylmethoxy, and pyrazin-2-ylethoxy; and
R⁴ is selected from ethynyl, thiazol-2-yl, 1H-pyrazol-4-yl, 1-methyl-1H-pyrazol-4-yl, and 1H-pyrazol-3-yl.

In some embodiments:
A′ is -L²-Ar²;
L² is —CH₂—;
Ar² is selected from 1,2,4-triazol-1-yl, 1H-pyrazol-1-yl, and 1H-imidazol-1-yl; each of which is optionally substituted by 1 or 2 independently selected C₁₋₆ alkyl groups;
R¹ is H;
R² is H;
R³ is selected from pyrazin-2-ylmethoxy, pyridin-3-ylmethoxy, pyrazin-2-ylethoxy, and pyrimidin-5-ylmethoxy; and
R³ is selected from —O—CH₂-Het and —OCH₂—CH₂-Het; wherein Het is a pyrazine ring, a pyridine ring, or a pyrimidine ring; which is optionally substituted by a C₁₋₆ alkyl group; and
R⁴ is selected from C₂₋₆ alkynyl, 1H-pyrazol-3-yl, 1H-pyrazol-4-yl, 1-methyl-1H-pyrazol-3-yl, 1-methyl-1H-pyrazol-4-yl, and 1-methyl-1H-pyrazol-5-yl.

In some embodiments:
A′ is -L²-Ar²;
L² is —CH₂−;
Ar² is selected from 1,2,4-triazol-1-yl, 1H-pyrazol-1-yl, and 1H-imidazol-1-yl;
R¹ is H;
R² is H;
R³ is selected from pyrazin-2-ylmethoxy, pyridin-3-ylmethoxy, pyrazin-2-ylethoxy, and pyrimidin-5-ylmethoxy; and
R⁴ is selected from ethynyl, 1H-pyrazol-3-yl, 1H-pyrazol-4-yl, 1-methyl-1H-pyrazol-3-yl, 1-methyl-1H-pyrazol-4-yl, and 1-methyl-1H-pyrazol-5-yl.

In some embodiments:
A′ is -L¹-Cy¹;
L¹ is selected from —CH₂— or —CH₂C(═O)—;
Cy¹ is morpholin-4-yl; which is optionally substituted by 1 or 2 independently selected C₁₋₆ alkyl groups;
R¹ is H;
R² is H;
R³ is selected from —O—CH₂-Het and —OCH₂—CH₂-Het; wherein Het is a pyrazine ring, a pyridine ring, or a pyrimidine ring; which is optionally substituted by a C₁₋₆ alkyl group; and
R⁴ is selected from C₂₋₆ alkynyl, 1H-pyrazol-4-yl, 1-methyl-1H-pyrazol-4-yl, and 1H-pyrazol-3-yl.

In some embodiments:
A′ is -L¹-Cy¹;
L¹ is selected from —CH₂— or —CH₂C(═O)—; Cy¹ is selected from morpholin-4-yl;
R¹ is H;
R² is H;
R³ is selected from pyrazin-2-ylmethoxy, pyridin-3-ylmethoxy, pyrimidin-5-ylmethoxy, and pyrazin-2-ylethoxy; and
R⁴ is selected from ethynyl, 1H-pyrazol-4-yl, 1-methyl-1H-pyrazol-4-yl, and 1H-pyrazol-3-yl.
   In some embodiments:

A′ is -L¹-Cy¹;
L¹ is —C(═O)—;
Cy¹ is selected from a morpholine ring; which is optionally substituted by 1 or 2 independently selected C₁₋₆ alkyl groups;
R¹ is H;
R² is H;
R³ is selected from —O—CH₂-Het and —OCH₂—CH₂-Het; wherein Het is a pyrazine ring; which is optionally substituted by a C₁₋₆ alkyl group; and
R⁴ is selected from cyano, methyl, ethynyl, cyclopropyl, 1-methyl-1H-pyrazol-4-yl, 1H-pyrazol-4-yl, and 1H-pyrazol-3-yl.

In some embodiments:
A′ is -L¹-Cy¹;
L¹ is —C(═O)—;
Cy¹ is selected from morpholin-4-yl
R¹ is H;
R² is H;
R³ is selected from pyrazin-2-ylmethoxy; and
R⁴ is selected from cyano, methyl, ethynyl, cyclopropyl, 1-methyl-1H-pyrazol-4-yl, 1H-pyrazol-4-yl, and 1H-pyrazol-3-yl.

In some embodiments:
A′ is -L¹-Cy¹;
L¹ is —O—;
Cy¹ is selected from tetrahydro-2H-pyran ring and a pyrrolidine ring; each of which is optionally substituted by 1 or 2 independently selected C₁₋₆ alkyl groups;
R¹ is selected from H and halogen;
R² is selected from H, halogen, and C₁₋₆ alkoxy;
R³ is selected from H, —O—CH₂-Het, and —O—Hy; wherein Het is a pyridine ring, which is optionally substituted by a C₁₋₆ alkyl group; Hy is piperidine ring, which is optionally substituted by a C₁₋₆ alkyl group; and
R⁴ is selected from halogen, C₂₋₆ alkenyl, and thiazol-2-yl.

In some embodiments:
A′ is -L¹-Cy¹;
L¹ is —O—;
Cy¹ is selected from tetrahydro-2H-pyran-4-yl and pyrrolidin-3-yl;
R¹ is selected from H and chloro;
R² is selected from H, chloro, fluoro, and methoxy;
R³ is selected from H, pyridin-3-ylmethoxy, N-methylpiperidin-4-yloxy, and piperidin-4-yloxy, and
R⁴ is selected from bromo, ethynyl, and thiazol-2-yl.

In some embodiments:

A′ is -L¹-Cy¹;
L¹ is a bond;
Cy¹ is selected from a 2-oxopyrrolidine ring, a piperidine ring, and a morpholine ring; each of which is optionally substituted by 1 or 2 independently selected C₁₋₆ alkyl groups;
R¹ is H;
R² is H;
R³ is selected from pyrazin-2-ylmethoxy and pyridin-3-ylmethoxy; and
R⁴ is selected from bromo, ethynyl, 1-methyl-1H-pyrazol-4-yl, 1H-pyrazol-4-yl, and 1H-pyrazol-3-yl.

In some embodiments:
A′ is -L¹-Cy¹;
L¹ is a bond;
Cy¹ is selected from 2-oxopyrrolidinyl, N-methylpiperidin-4-yl, and morpholin-4-yl;
R¹ is H;
R² is H;
R³ is selected from pyrazin-2-ylmethoxy and pyridin-3-ylmethoxy; and
R⁴ is selected from bromo, ethynyl, 1-methyl-1H-pyrazol-4-yl, 1H-pyrazol-4-yl, and 1H-pyrazol-3-yl.

In some embodiments, Ar′ is a moiety of Group (B):

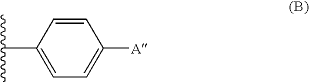

In some embodiments, A″ is selected from -L^{2a}-Ar^{2a}. In some embodiments, A″ is selected from —Cy^{1a}.

In some embodiments:
A″ is —Cy^{1a};
Cy^{1a} is selected from a 2-oxopyrrolidine ring and a piperidine ring; each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
R¹ is H;
R² is H;
R³ is selected from —O—CH₂-Het and —O-Hy; wherein Het is 6-membered heteroaryl which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and
R⁴ is selected from C₂₋₆ alkynyl and a thiazole ring; where said thiazole ring is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups.

In some embodiments:
A″ is —Cy^{1a};
Cy^{1a} is selected from 2-oxopyrrolidinyl and piperidin-4-yl;
R¹ is H;
R² is H;
R³ is selected from pyridin-3-ylmethoxy, piperidin-4-yloxy and N-methylpiperidin-4-yloxy; and
R⁴ is selected from ethynyl and thiazol-2-yl.

In some embodiments:
A″ is -L^{2a}Ar^{2a};
L^{2a} is a bond;
R¹ is H;
R² is H;
Ar^{2a} is selected from a pyrazole ring and a pyrimidine ring; each of which is optionally substituted with 1 or 2 groups independently selected from hydroxyl, C₁₋₆ alkyl, C₁₋₄ alkoxy, amino, C₁₋₄ alkylamino, and di-C₁₋₄ alkylamino;
R³ is —O—Hy; wherein Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and
R⁴ is selected from a thiazole ring and a pyrazole ring; each of which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups.

In some embodiments:
A″ is -L^{2a}Ar^{2a};
L^{2a} is a bond;
Ar^{2a} is selected from 1-methyl-1H-pyrazol-4-yl, 6-methyl-1H-pyrazol-3-yl, 2-methylpyrimidin-5-yl, pyridin-4-yl, pyridin-3-yl, 6-methoxypyridin-3-yl, 2-methoxypyridin-3-yl and 1,2,4-triazol-5-yl;
R¹ is H;
R² is H;
R³ is selected from piperidin-4-yloxy and N-methylpiperidin-4-yloxy; and
R⁴ is selected from 1H-pyrazol-4-yl and thiazol-2-yl.

In some embodiments:
A″ is -L^{2a}Ar^{2a};
L^{2a} is —CH₂—;
Ar^{2a} is selected from a pyrazole ring; which is optionally substituted with 1 or 2 groups independently selected from hydroxyl, C₁₋₆ alkyl, C₁₋₄ alkoxy, amino, C₁₋₄ alkylamino, and di-C₁₋₄ alkylamino;

R³ is —O—Hy; wherein Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and

R⁴ is selected from a thiazole ring and a pyridine ring; each of which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups.

In some embodiments:
A″ is -L^{2a}A^{2a};
L^{2a} is CH₂—,
Ar^{2a} is selected from 1H-pyrazol-1-yl and 1H-pyrazol-5-yl,
R¹ is H;
R² is H;
R³ is selected from piperidin-4-yloxy and N-methylpiperidin-4-yloxy; and
R⁴ is selected from pyridin-3-yl, 4-(hydroxymethyl)thiazol-2-yl and thiazol-2-yl.

In some embodiments:
A″ is -L^{2a}A^{2a};
L^{2a} is —O—:
Ar^{2a} is selected from a pyrimidine ring; which is optionally substituted with 1 or 2 groups independently selected from hydroxyl, C₁₋₆ alkyl, C₁₋₄ alkoxy, amino, C₁₋₄ alkylamino, and di-C₁₋₄ alkylamino;
R¹ is H;
R² is H;
R³ is —O—Hy; wherein Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and
R⁴ is a thiazole ring; which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups.

In some embodiments:
A″ is -L^{2a}Ar^{2a};
L^{2a} is —O—:
Ar^{2a} is selected from pyrimidin-2-yl;
R¹ is H;
R² is H;
R³ is selected from piperidin-4-yloxy and N-methylpiperidin-4-yloxy; and
R⁴ is selected from thiazol-2-yl.

A compound according to claim 1, or pharmaceutically acceptable salt thereof, wherein Ar¹ is a moiety of Group (C):

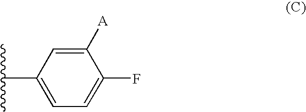

In some embodiments, A is pyrazol-4-yl, which is substituted by a methyl group.

In some embodiments, the present invention provides a compound of Formula (Ina) or (IVa):

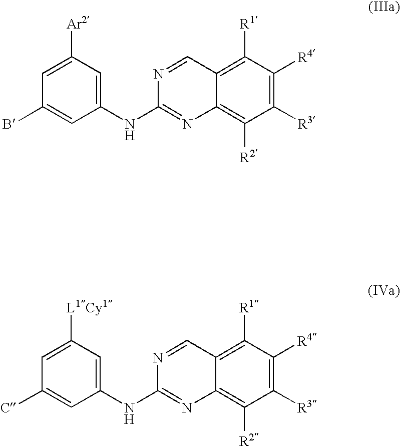

or pharmaceutically acceptable salt thereof, wherein:

R^{1′} and R^{1″} are each independently selected from H and halogen;

R^{2′} and R^{2″} are each independently selected from H, halogen, and C₁₋₆ alkoxy;

R^{3′} and R^{3″} are each independently selected from H, halogen, and C₃₋₅ heterocycloalkyloxy;

R^{4′} and R^{4″} are each independently selected from H, halogen, cyano, C₁₋₆ alkyl, C₂₋₆ alkynyl, C₃₋₆ cycloalkyl, 5-membered heteroaryl and 6-membered heteroaryl; wherein said C₃₋₆ cycloalkyl, 5-membered heteroaryl and 6-membered heteroaryl are each optionally substituted by 1 or 2 groups independently selected from C₁₋₆ alkyl and C₁₋₆ alkoxy;

B′ is selected from -L^{1′}Cy^{1′} and B″;

B″ is selected from C₁₋₆ alkyl and C₁₋₆ alkoxy; which are each substituted by 1, 2, or 3 groups independently selected from hydroxyl, amino, C₁₋₆ alkylamino, di-C₁₋₆ alkylamino, and C₁₋₆ alkylcarbonylamino; wherein said C₁₋₆ alkylamino, di-C₁₋₆ alkylamino, and C₁₋₆ alkylcarbonylamino are each optionally substituted by 1 or 2 groups independently selected from C₁₋₄ alkoxy, amino, C₁₋₄ alkylamino, and di-C₁₋₄ alkylamino;

L^{1′} is selected from —OCH₂CH₂—, —C(═O)—, —OCH₂CH₂NHC(═O)—, —OCH₂CH₂NHC(═O)CH₂—, —O—, —CH₂— and —CH(CH₃)—, wherein the right end of the linker is attached to Cy^{1′}; Cy^{1′} is selected from C₃₋₅ heterocycloalkyl, which is optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, alkylamino, and 5-membered heterocycloalkyl;

Ar^{2′} is selected from an oxazole ring, a pyrazole ring, a pyrimidine ring, and a pyridine ring, each of which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino;

L^{1″} is —CH₂—;

Cy^{1″} is selected from 5-membered heterocycloalkyl and 6-membered heterocycloalkyl; and

C″ is carbamyl;

provided that:

(i) when R¹ is H, R² is H, R³ is H, Ar^{2′} is 1-methyl-1H-pyrazole, and B′ is selected from morpholin-4-ylmethyl, 2-aminoethoxy, 2-amino-3-hydroxylpropoxy, and 2-dimethylaminoethoxy, then R⁴ is not bromo;

(ii) when Ar^{2′} is 1-methyl-1H-pyrazol-4-yl, B′ is B″, B″ is other than substituted C₁₋₆ alkyl, and R^{4′} is ethynyl, then at least one of R^{1′, R}², or R³ is not H;

(iii) when R^{1′} is H, R^{2′} is H, R^{3′} is H, R^{4′} is ethynyl, and Ar^{2′} is pyrimidin-5-yl, then B′ is not B″ or L^{1′} is not —OCH₂CH₂NHC(═O)—;

(iv) when R^{1′} is H, R^{2′} is H, R^{3′} is H, R^{4′} is ethynyl, and Ar^{2′} is 1-methyl-1H-pyrazol-4-yl, then B′ is not selected from 2-(pyrrolidinyl)ethoxy, 2-(4-methylpiperazinyl)ethoxy, morpholin-4-ylmethyl, 2-(methoxycarbonyl)pyrrolidin-3-yloxy, and pyrrolidin-3-yloxy;

(v) when R^{1′} is H, R^{2′} is H, R^{3′} is H, R^{4′} is ethynyl, L^{1′} is —OCH₂CH₂NHC(═O)CH₂— or —OCH₂CH₂NHC(═O)—, then Cy¹ is not selected from tetrahydro-2H-pyran-4-yl, a pyrrolidine ring optionally substituted with 1 or 2 groups independently selected from methyl, fluoro, and hydroxyl group, a piperidine ring optionally substituted with 1 or 2 groups independently selected from methyl, fluoro, and hydroxyl group, tetrahydrofuran-2-yl, and morpholin-4-yl, and Ar^{2′} is 1-methyl-1H-pyrazol-4-yl;

(vi) when R^{1′} is H, R^{2′} is H, R^{3′} is piperidin-4-yloxy, then Ar^{2′} is not oxazol-4-yl;

(vii) when R^{1′} is H, R^{2′} is H, R^{3′} is H, then B′ is morpholin-4-yl, and Ar^{2′} is pyridin-3-yl, pyridin-4-yl, 3-fluoropyridin-4-yl, and 1H-pyrazol-4-yl, then R^{4′} is not ethynyl; and

(viii) when -L^{1″}Cy^{1″} is morpholin-4-ylmethyl and C″ is carbamyl, then R⁴ is not bromo.

In some embodiments, the present invention provides a compound of Formula (IIa).

In some embodiments, the present invention provides a compound of Formula (IVa).

In some embodiments, B′ is selected from in some embodiments, B′ is B″.

In some embodiments, B″ is C₁₋₆ alkyl which is substituted by 1 or 2 groups independently selected from hydroxyl, amino, C₁₋₆ alkylamino, di-C₁₋₆ alkylamino, and C₁₋₆ alkylcarbonylamino; wherein said C₁₋₆ alkylamino, di-C₁₋₆ alkylamino, and C₁₋₆ alkylcarbonylamino are each optionally substituted by 1 or 2 groups independently selected from C₁₋₄ alkoxy, amino, C₁₋₄ alkylamino, and di-C₁₋₄ alkylamino. In some embodiments, B″ is C₁₋₆ alkoxy which is substituted by 1 or 2 groups independently selected from hydroxyl, amino, C₁₋₆ alkylamino, alkylamino, and C₁₋₆ alkylcarbonylamino; wherein said C₁₋₆ alkylamino, di-C₁₋₆ alkylamino, and C₁₋₆ alkylcarbonylamino are each optionally substituted by 1 or 2 groups independently selected from C₁₋₄ alkoxy, amino, C₁₋₄ alkylamino, and di-C₁₋₄ alkylamino. In some embodiments, B″ is selected from 2-aminethoxy, 2-hydroxyethoxy, 2-(N,N-dimethylamino)ethoxy, 2-(N,N-diethylamino)ethoxy, 2-(acetylamino)ethoxy, 2-(3-methoxypropionylamino)ethoxy, 2-(N,N-dimethylaminoacetylamino)ethoxy, and N,N-dimethylaminomethyl.

In some embodiments, L^{1′} is selected from —OCH₂CH₂—, —C(═O)—, —OCH₂CH₂NHC(═O)—, —OCH₂CH₂NHC(═O)CH₂—, —O—, —CH₂— and —CH(CH₃)—, wherein the right end of the linker is attached to Cy¹. In some embodiments, —OCH₂CH₂—. In some embodiments, L^{1′} is —C(═O)—. In some embodiments, is —OCH₂CH₂NHC(═O)—.

In some embodiments, L^{1′} is —OCH₂CH₂NHC(═O)CH₂—. In some embodiments, L^{1′} is —O—. In some embodiments, L^{1′} is —CH₂—. In some embodiments, L^{1′} is —CH(CH₃)—. In some embodiments, L^{1′} is selected from —OCH₂CH₂—, —C(═O)—, —OCH₂CH₂NHC(═O)— and —OCH₂CH₂NHC(═O)CH₂—. In some embodiments, L^{1′} is selected from —O—. In some embodiments, L^{1′} is selected from —CH₂— and —CH(CH₃)—.

In some embodiments, Cy^{1′} is selected from an azetidine ring, a piperazine ring, a imidazolidine ring, a pyrrolidine ring, a piperidine ring, a tetrahydrofuran ring, a morpholine ring, a 2-oxopiperazinyl, and a 2-oxa-5-azabicyclo[2.2.1]heptane ring; each of which is optionally substituted by hydroxyl, halogen, C₁₋₆ alkyl, C₁₋₆ haloalkyl, C₁₋₆ alkoxycarbonyl, and C₁₋₆ alkylcarbonyl; wherein said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl. In some embodiments, Cy^{1′} is selected from S,S-dioxothiomorpholin-4-yl, morpholin-4-yl, 2-methylmorpholin-4-yl, 2,6-dimethylmorpholin-4-yl, 2-hydroxymethylmorpholin-4-yl, 2-hydroxylmorpholin-4-yl, 3-hydroxyazetidinyl, 3-hydroxypyrrolidinyl, pyrrolidin-3-yl, 3-fluoropyrrolidinyl, 3,3-difluoropyrrolidinyl, piperidin-4-yl, 3-fluoropiperidinyl, 4-trifluoromethylpiperidinyl, 4,4-difluoropiperidinyl, 4-methylpiperazinyl, and 2-oxopiperazinyl, 2-oxa-5-azabicyclo[2.2.1]heptan-5-yl.

In some embodiments, Ar^{2′} is selected from an oxazole ring, a pyrazole ring, a pyridine ring, and a pyrimidine ring; each of which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino. In some embodiments, Ar^{2′} is selected from an oxazole ring, a pyrazole ring, a pyrimidine ring, and a pyridine ring, each of which is optionally substituted by 1 or 2 groups independently selected from amino and C₁₋₄ alkyl.

In some embodiments, Ar^{2′} is selected from an oxazole ring, which is optionally substituted by 1 or 2 groups independently selected from amino and C₁₋₄ alkyl. In some embodiments, Ar^{2′} is a pyrazole ring, which is optionally substituted by 1 or 2 groups independently selected from amino and C₁₋₄ alkyl. In some embodiments, Ar^{2′} is a pyrimidine ring, which is optionally substituted by 1 or 2 groups independently selected from amino and C₁₋₄ alkyl. In some embodiments, Ar^{2′} is a pyridine ring, which is optionally substituted by 1 or 2 groups independently selected from amino and C₁₋₄ alkyl. In some embodiments, Ar^{2′} is selected from an oxazole ring, which is substituted by 1 or 2 groups independently selected from amino and C₁₋₄ alkyl. In some embodiments, Ar^{2′} is a pyrazole ring, which is substituted by 1 or 2 groups independently selected from amino and C₁₋₄ alkyl. In some embodiments, Ar^{2′} is a pyrimidine ring, which is substituted by 1 or 2 groups independently selected from amino and C₁₋₄ alkyl. In some embodiments, Ar^{2′} is a pyridine ring, which is substituted by 1 or 2 groups independently selected from amino and C₁₋₄ alkyl. In some embodiments, Ar^{2′} is selected from oxazol-2-yl, 1-methylpyrazol-4-yl, 1-methyl-1H-pyrazol-4-yl, pyrazol-4-yl, 1-methyl-1H-pyrazol-4-yl, 1-ethyl-1H-pyrazol-4-yl, 2-aminopyridin-4-yl, 6-aminopyridin-3-yl, and pyrimidin-5-yl.

Cy^{1′} is selected from 5-membered heterocycloalkyl. In some embodiments, Cy^{1″} is morpholin-4-yl. In some embodiments, Cy^{1″} is a morpholine ring.

In some embodiments, R^{1′} is selected from H and chloro. In some embodiments, R^{1′} is H. In some embodiments, R^{1′} is chloro.

In some embodiments, R^{1″} is selected from H and chloro. In some embodiments, R^{1″} is H. In some embodiments, R^{1″} is chloro.

In some embodiments, R^{2′} is selected from H, fluoro, chloro, and methoxy. In some embodiments, R^{2′} is selected from H and methoxy. In some embodiments, R^{2′} is H. In some embodiments, R^{2′} is fluoro. In some embodiments, R^{2′} is chloro. In some embodiments, R^{2′} is methoxy. In some embodiments, R^{2′} is halogen. In some embodiments, R^{2′} is C₁₋₆ alkoxy.

In some embodiments, R^{2″} is selected from H, fluoro, chloro, and methoxy. In some embodiments, R^{2″} is selected from H and methoxy. In some embodiments, R^{2″} is H. In some embodiments, R^{2′} is fluoro. In some embodiments, R^{2″} is chloro. In some embodiments, R^{2″} is methoxy. In some embodiments, R^{2″} is halogen. In some embodiments, R^{2″} is C₁₋₆ alkoxy.

In some embodiments, R^{3′} is H.

In some embodiments, at least one of R^{1′}, R^{2′} or R^{3′} is not H. In some embodiments, at least one of R^{1″}, R^{2″} or R^{3″} is not H.

In some embodiments, R^{4′} is selected from chloro, bromo, and ethynyl. In some embodiments, R^{4′} is selected from bromo and ethynyl. In some embodiments, R^{4′} is selected from H, bromo, chloro, cyano, methyl, ethynyl, and cyclopropyl. In some embodiments, R^{4′} is selected from H, bromo, ethynyl, cyclopropyl, and thiazol-2-yl. In some embodiments, R^{4′} is selected from H, halogen, cyano, ethynyl, C₃₋₆ cycloalkyl, and a thiazole ring; wherein said C₃₋₆ cycloalkyl and a thiazole ring are each optionally substituted by 1 or 2 groups independently selected from C₁₋₆ alkyl and C₁₋₆ alkoxy.

In some embodiments, R^{4′} is H. In some embodiments, R^{4′} is halogen. In some embodiments, R^{4′} is cyano. In some embodiments, R^{4′} is C₁₋₆ alkyl. In some embodiments, R^{4′} is C₂₋₆ alkynyl. In some embodiments, R^{4′} is C₃₋₆ cycloalkyl, which is optionally substituted by 1 or 2 groups independently selected from C₁₋₆ alkyl and C₁₋₆ alkoxy. In some embodiments, R^{4′} is 5-membered heteroaryl, which is optionally substituted by 1 or 2 groups independently selected from C₁₋₆ alkyl and C₁₋₆ alkoxy. In some embodiments, R^{4′} is 6-membered heteroaryl, which is optionally substituted by 1 or 2 groups independently selected from C₁₋₆ alkyl and C₁₋₆ alkoxy. In some embodiments, R^{4′} is methyl. In some embodiments, R^{4′} is ethynyl. In some embodiments, R^{4′} is cyclopropyl, which is optionally substituted by 1 or 2 groups independently selected from C₁₋₆ alkyl and C₁₋₆ alkoxy. In some embodiments, R^{4′} is thiazol-2-yl, which is optionally substituted by 1 or 2 groups independently selected from C₁₋₆ alkyl and C₁₋₆ alkoxy. In some embodiments, R^{4′} is chloro.

In some embodiments, R^{4″} is H. In some embodiments, R^{4″} is halogen. In some embodiments, R^{4′} is cyano. In some embodiments, R^{4″} is C₁₋₆ alkyl. In some embodiments, R^{4″} is C₂₋₆ alkynyl. In some embodiments, R^{4″} is C₃₋₆ cycloalkyl, which is optionally substituted by 1 or 2 groups independently selected from C₁₋₆ alkyl and C₁₋₆ alkoxy. In some embodiments, R^{4″} is 5-membered heteroaryl, which is optionally substituted by 1 or 2 groups independently selected from C₁₋₆ alkyl and C₁₋₆ alkoxy. In some embodiments, R^{4″} is 6-membered heteroaryl, which is optionally substituted by 1 or 2 groups independently selected from C₁₋₆ alkyl and C₁₋₆ alkoxy. In some embodiments, R^{4″} is methyl. In some embodiments, R^{4″} is ethynyl. In some embodiments, R^{4″} is cyclopropyl, which is optionally substituted by 1 or 2 groups independently selected from C₁₋₆ alkyl and C₁₋₆ alkoxy. In some embodiments, R^{4″} is thiazol-2-yl, which is optionally substituted by 1 or 2 groups independently selected from C₁₋₆ alkyl and C₁₋₆ alkoxy. In some embodiments, R^{4″} is chloro.

In some embodiments, R^{4″} is selected from chloro, bromo, and ethynyl. In some embodiments, R^{4″} is selected from bromo and ethynyl. In some embodiments, R^{4″} is selected from H, bromo, chloro, cyano, methyl, ethynyl, and cyclopropyl. In some embodiments, R^{4″} is selected from H, bromo, ethynyl, cyclopropyl, and thiazol-2-yl. In some embodiments, R^{4″} is selected from H, halogen, cyano, ethynyl, C₃₋₆ cycloalkyl, and a thiazole ring; wherein said C₃₋₆ cycloalkyl and a thiazole ring are each optionally substituted by 1 or 2 groups independently selected from C₁₋₆ alkyl and C₁₋₆ alkoxy.

In some embodiments:

R^{1′} is selected from H and halogen;

R^{2′} is selected from H, halogen, and C₁₋₆ alkoxy;

R^{3′} is selected from H and halogen;

R^{4′} is selected from H, halogen, cyano, ethynyl, C₃₋₆ cycloalkyl, and a thiazole ring; wherein said C₃₋₆ cycloalkyl and a thiazole ring are each optionally substituted by 1 or 2 groups independently selected from C₁₋₆ alkyl and C₁₋₆ alkoxy;

Cy^{1′} is selected from an azetidine ring, a piperazine ring, a imidazolidine ring, a pyrrolidine ring, a piperidine ring, a tetrahydrofuran ring, a morpholine ring, a 2-oxopiperazinyl, and a 2-oxa-5-azabicyclo[2.2.1]heptane ring; each of which is optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl.

Cy^{1″} is a morpholine ring;

Ar^{2′} is selected from an oxazole ring, a pyrazole ring, a pyridine ring, and a pyrimidine ring; each of which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino; and

B″ is selected from C₁₋₆ alkyl and C₁₋₆ alkoxy; which are each substituted by 1, 2, or 3 groups independently selected from hydroxyl, amino, C₁₋₆ alkylamino, alkylamino, and C₁₋₆ alkylcarbonylamino; wherein said C₁₋₆ alkylamino, di-C₁₋₆ alkylamino, and C₁₋₆ alkylcarbonylamino are each optionally substituted by 1 or 2 groups independently selected from C₁₋₄ alkoxy, amino, C₁₋₄ alkylamino, and di-C₁₋₄ alkylamino.

In some embodiments, the compound has Formula (Ma), wherein:
C″ is carbamyl; and
Cy^{1″} is morpholin-4-yl.

In some embodiments, the compound has Formula (IIIa), wherein:

R^{1′} is selected from H and halogen;

R^{2′} is selected from H, halogen, and C₁₋₆ alkoxy;

R^{3′} is H;

R^{4′} is selected from halogen and C₂₋₆ alkynyl;

B′ is selected from B″;

B″ is selected from C₁₋₆ alkoxy; which is substituted by 1, 2, or 3 groups independently selected from hydroxyl, amino, C₁₋₆ alkylamino, di-C₁₋₆ alkylamino, and C₁₋₆ alkylcarbonylamino; wherein said C₁₋₆ alkylamino, di-C₁₋₆ alkylamino, and C₁₋₆ alkylcarbonylamino are each optionally substituted by 1 or 2 groups independently selected from C₁₋₄ alkoxy, amino, C₁₋₄ alkylamino, and di-C₁₋₄ alkylamino; and

Ar^{2′} is selected from an oxazole ring and a pyrazole ring; each of which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino.

In some embodiments, the compound has Formula (IIIa), wherein:
R^{1′} is selected from H and chloro;
R^{2′} is selected from H, fluoro, chloro, and methoxy;
R^{3′} is selected from H;
R^{4′} is selected from chloro, bromo, and ethynyl;
Ar^{2′} is selected from oxazol-2-yl and 1-methylpyrazol-4-yl,
B′ is B″; and
B″ is selected from 2-aminethoxy, 2-hydroxyethoxy, 2-(N,N-dimethylamino)ethoxy, 2-(N,N-diethylamino)ethoxy, 2-(acetylamino)ethoxy, 2-(3-methoxypropionylamino)ethoxy, 2-(N,N-dimethylaminoacetylamino)ethoxy, and N,N-dimethylaminomethyl.

In some embodiments, the compound has Formula (Ma), wherein:

R^{1′} is H;

R^{2′} is C₁₋₆ alkoxy;

R^{3′} is H;

R^{4′} is halogen and C₂₋₆ alkynyl;

B′ is -L^{1′}Cy^{1′};

L^{1′} is selected from —OCH₂CH₂—, —C(═O)—, —OCH₂CH₂NHC(═O)—, and —OCH₂CH₂NHC(═O)CH₂—; wherein the right end of the linker is attached to Cy^{1′};

Cy^{1′} is selected from a azetidine ring, a piperazine ring, a imidazolidine ring, a pyrrolidine ring, and a pyrrolidine ring; wherein said azetidine ring, piperazine ring, and imidazolidine ring are each optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl; and wherein said pyrrolidine ring is substituted with a C₁₋₆ alkoxycarbonyl group; and

Ar^{2′} is a pyrazole ring; which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino.

In some embodiments, the compound has Formula (IIIa), wherein:
R^{1′} is H;
R^{2′} is selected from H and methoxy;
R^{3′} is H;
R^{4′} is selected from bromo and ethynyl;
B′ is selected from -L^{1′}Cy^{1′};
L^{1′} is selected from —OCH₂CH₂—, —C(═O)—, —OCH₂CH₂NHC(═O)— and —OCH₂CH₂NHC(═O)CH₂—;
Cy^{1′} is selected from azetidinyl, N-methylpiperazinyl, 1-methylimidazolidin-3-yl, pyrrolidinyl, and pyrrolidinyl substituted with tert-butoxycarbonyl; and
Ar^{2′} is selected from 1-methyl-1H-pyrazol-4-yl.

In some embodiments, the compound has Formula (IIIa), wherein:

R^{1′} is H;

R^{2′} is selected from H, halogen, and C₁₋₆ alkoxy;

R^{3′} is H;

R^{4′} is selected from H, halogen, cyano, C₁₋₆ alkyl, C₂₋₆ alkynyl, and C₃₋₆ cycloalkyl; wherein said C₃₋₆ cycloalkyl is optionally substituted by 1 or 2 groups independently selected from C₁₋₆ alkyl and C₁₋₆ alkoxy;

B′ is -L^{1′}Cy^{1′};

L^{1′} is —O—;

Cy^{1′} is selected from a pyrrolidine ring, a piperidine ring, a tetrahydrofuran ring, and an azetidine ring; each of which is optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl; and

Ar^{2′} is selected from a pyrazole ring; which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino.

In some embodiments, the compound has Formula (IIIa), wherein:

R^{1′} is H;

R^{2′} is selected from H, halogen, and C₁₋₆ alkoxy;

R^{3′} is H;

R^{4′} is selected from halogen, cyano, C₁₋₆ alkyl, C₂₋₆ alkynyl, and C₃₋₆ cycloalkyl; wherein said C₃₋₆ cycloalkyl is optionally substituted by 1 or 2 groups independently selected from C₁₋₆ alkyl and C₁₋₆ alkoxy;

B′ is -L^{1′}Cy^{1′};

L^{1′} is —O—;

Cy^{1′} is selected from a pyrrolidine ring, a piperidine ring, a tetrahydrofuran ring, and an azetidine ring; each of which is optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl; and

Ar^{2′} is selected from a pyrazole ring; which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino.

In some embodiments, the compound has Formula (IIIa), wherein:
R^{1′} is H;
R^{2′} is selected from H, fluoro, chloro, and methoxy;
R^{3′} is H;
R^{4′} is selected from H, bromo, chloro, cyano, methyl, ethynyl, and cyclopropyl;
B′ is selected from -L^{1′}Cy^{1′};
L^{1′} is selected from —O—;
Cy^{1′} is selected from pyrrolidin-3-yl, N-methylpyrrolidin-3-yl, N-ethylpyrrolidin-3-yl, N-isopropylpyrrolidin-3-yl, N-(dimethylaminoacetyl)pyrrolidin-3-yl, N-(pyrrolidinylacetyl)pyrrolidin-3-yl, N-(2-hydroxyethyl)pyrrolidin-3-yl, piperidin-4-yl, tetrahydrofuran-3-yl, azetidin-3-yl, N-methylazetidin-3-yl, N-ethylazetidin-3-yl, N-isopropylazetidin-3-yl, N-(dimethylaminoacetyl)azetidin-3-yl, N-(pyrrolidinylacetyl)azetidin-3-yl, and N-(2,2,2-trifluoroethyl)azetidin-3-yl; and
Ar^{2′} is 1-methyl-1H-pyrazol-4-yl.

In some embodiments, the compound has Formula (IIIa), wherein:

R^{1′} is selected from H and halogen;

R^{2′} and R^{2″} are each independently selected from H, halogen, and C₁₋₆ alkoxy;

R^{3′} is H;

R^{4′} and R^{4″} are each independently selected from H, halogen, C₂₋₆ alkynyl, and a thiazole ring; wherein said C₃₋₆ cycloalkyl and thiazole ring are each optionally substituted by 1 or 2 groups independently selected from C₁₋₆ alkyl and C₁₋₆ alkoxy;

B′ is -L^{1′}Cy^{1′};

L^{1′} is selected from —CH₂— and —CH(CH₃)—;

Cy^{1′} is selected from a S,S-dioxothiomorpholine ring, a morpholine ring, an azetidine ring, a pyrrolidine ring, a piperidine ring, and a 2-oxa-5-azabicyclo[2.2.1]heptane ring; each of which is optionally substituted by hydroxyl, halogen, C₁₋₆ alkyl, C₁₋₆ haloalkyl, C₁₋₆ alkoxycarbonyl, and C₁₋₆ alkylcarbonyl; where said C₁₋₆ alkyl, C₁₋₆ haloalkyl, and C₁₋₆ alkylcarbonyl are each optionally substituted by a group selected from hydroxyl, amino, C₁₋₄ alkylamino, di-C₁₋₄-alkylamino, and 5-membered heterocycloalkyl; and

A^{2′} is selected from a pyrazole ring, a pyrimidine ring, and a pyridine ring, each of which is optionally substituted by 1 or 2 groups independently selected from hydroxyl, C₁₋₆ alkyl, C₁₋₆ alkoxy, amino, C₁₋₄ alkylamino, and di-C₁₋₄ alkylamino.

In some embodiments, the compound has Formula (IIIa), wherein:
R^{1′} is selected from H and chloro;
R^{2′} is selected from H, fluoro, chloro, and methoxy;
R^{3′} is selected from H;
R^{4′} is selected from H, bromo, ethynyl, cyclopropyl, and thiazol-2-yl,
B′ is selected from -L^{1′}Cy^{1′};
L^{1′} is selected from —CH₂— and —CH(CH₃)—;

Cy^{1′} is selected from S,S-dioxothiomorpholin-4-yl, morpholin-4-yl, 2-methylmorpholin-4-yl, 2,6-dimethylmorpholin-4-yl, 2-hydroxymethylmorpholin-4-yl, 2-hydroxylmorpholin-4-yl, 3-hydroxyazetidinyl, 3-hydroxypyrrolidinyl, pyrrolidin-3-yl, 3-fluoropyrrolidinyl, 3,3-difluoropyrrolidinyl, piperidin-4-yl, 3-fluoropiperidinyl, 4-trifluoromethylpiperidinyl, 4,4-difluoropiperidinyl, 4-methylpiperazinyl, and 2-oxopiperazinyl, 2-oxa-5-azabicyclo[2.2.1]heptan-5-yl; and
Ar^{2′} is selected from pyrazol-4-yl, 1-methyl-1H-pyrazol-4-yl, 1-ethyl-1H-pyrazol-4-yl, 2-aminopyridin-4-yl, 6-aminopyridin-3-yl, and pyrimidin-5-yl.

In some embodiments, the compound is selected from one of the following:

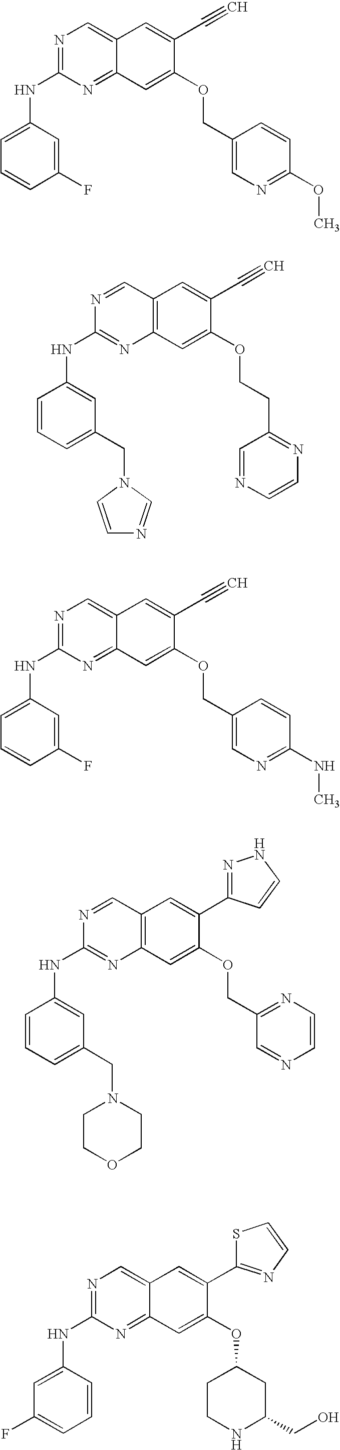

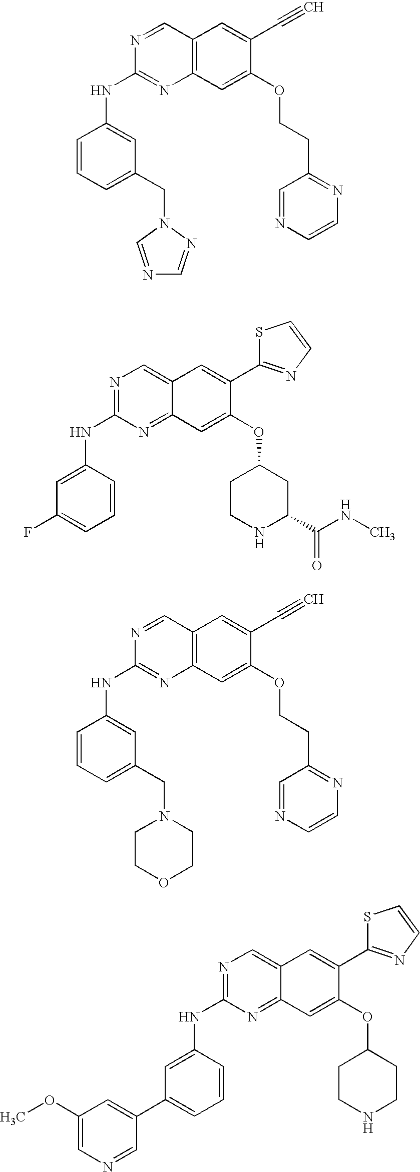

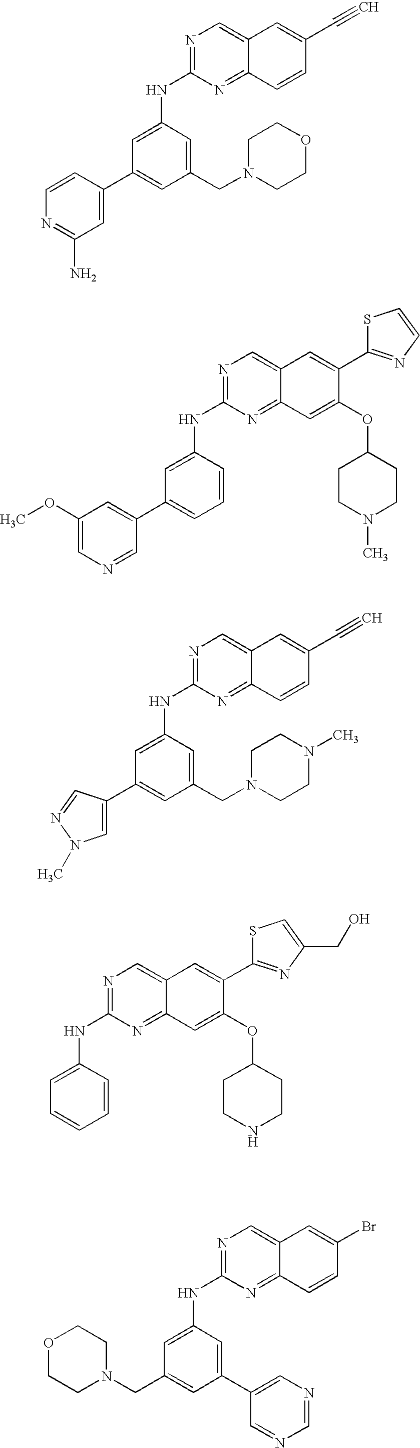

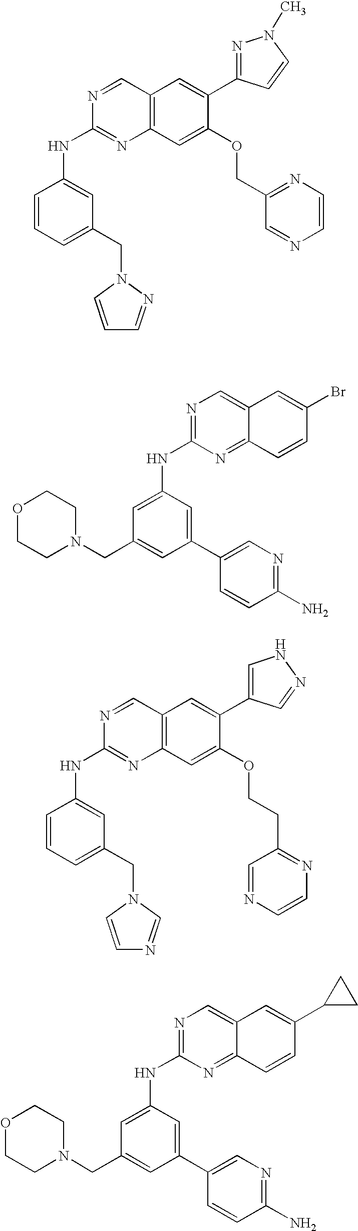

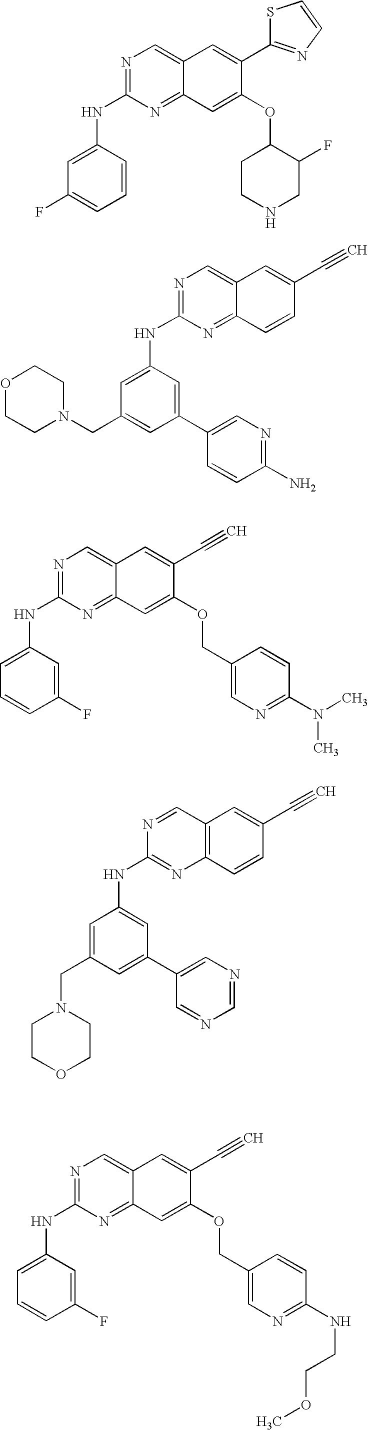

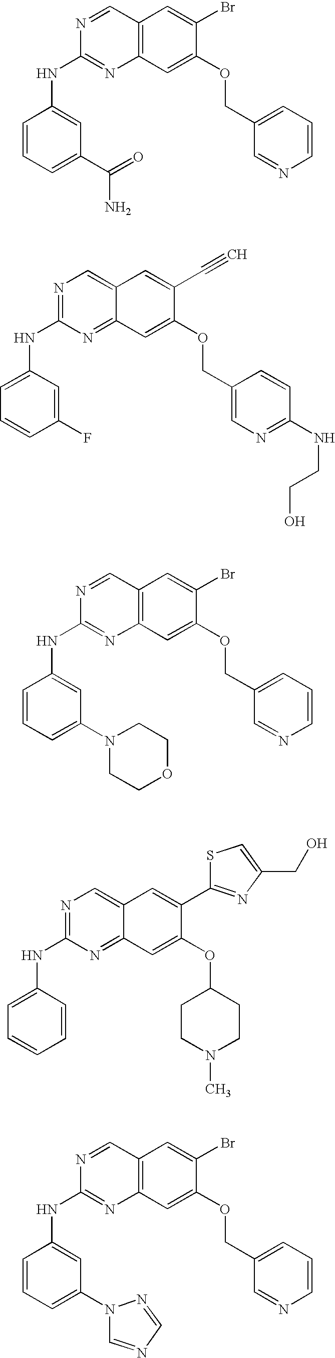

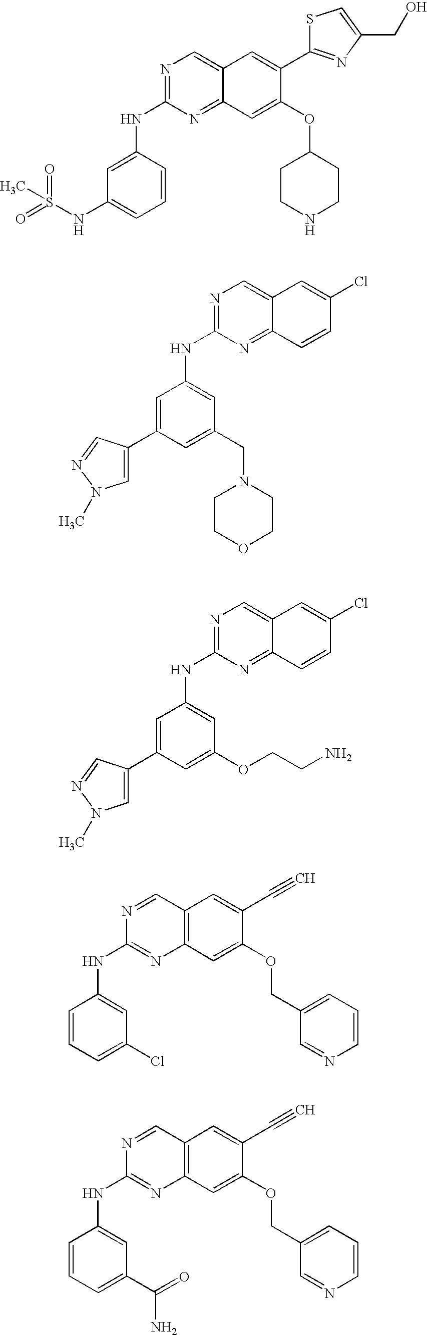

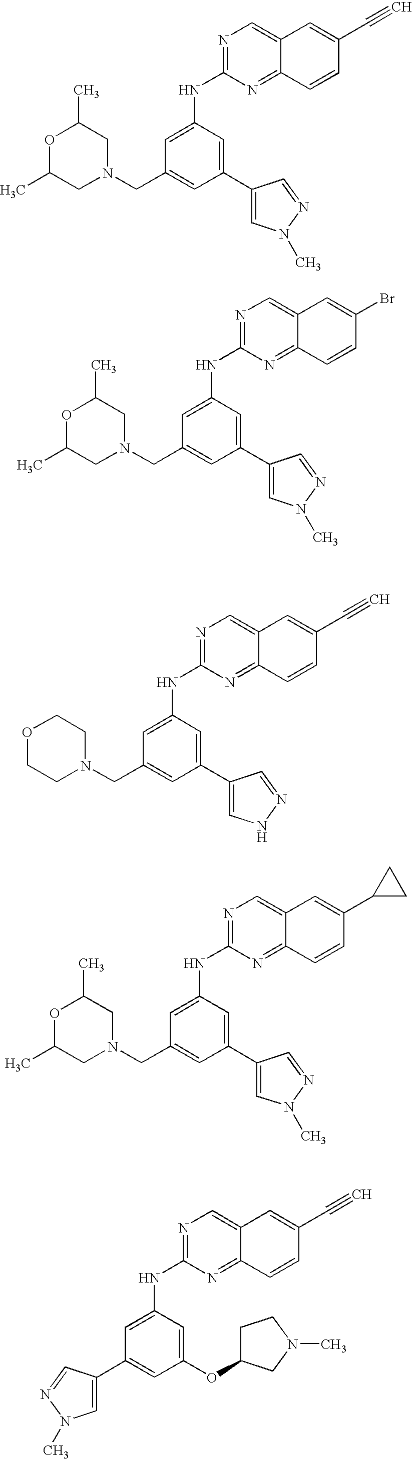

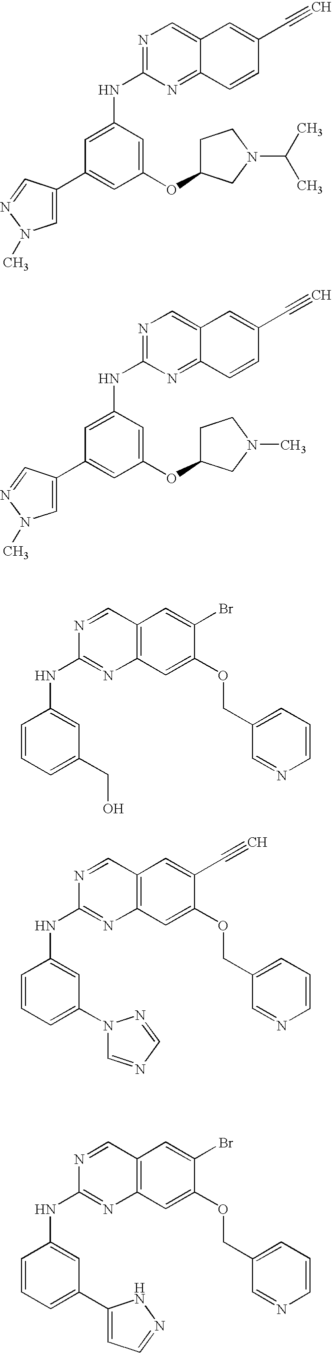

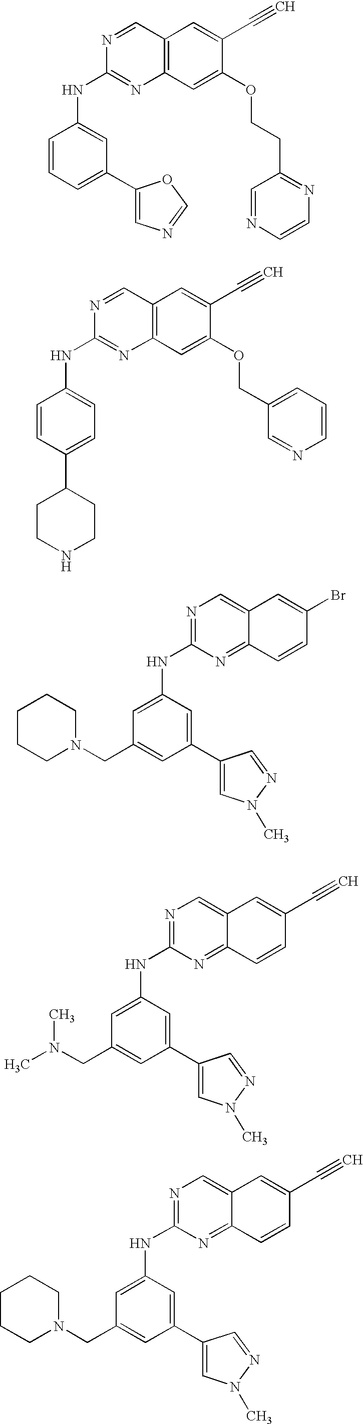

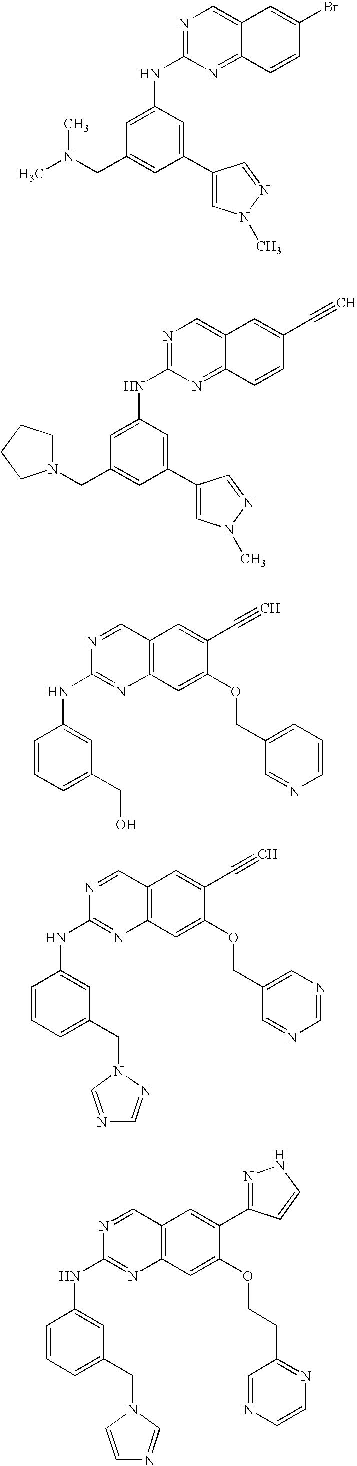

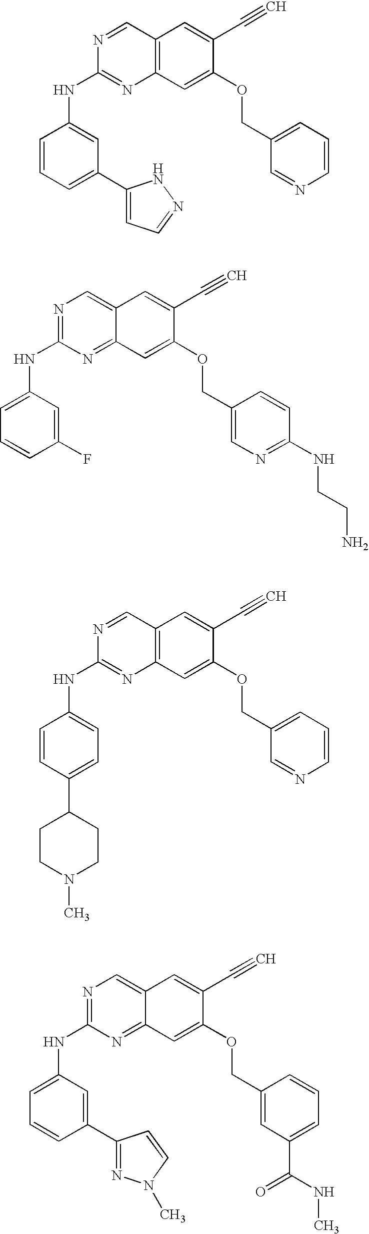

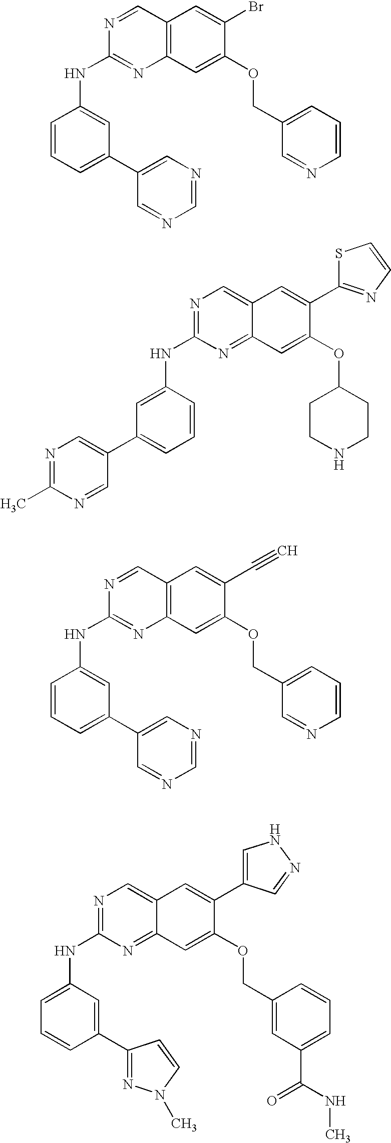

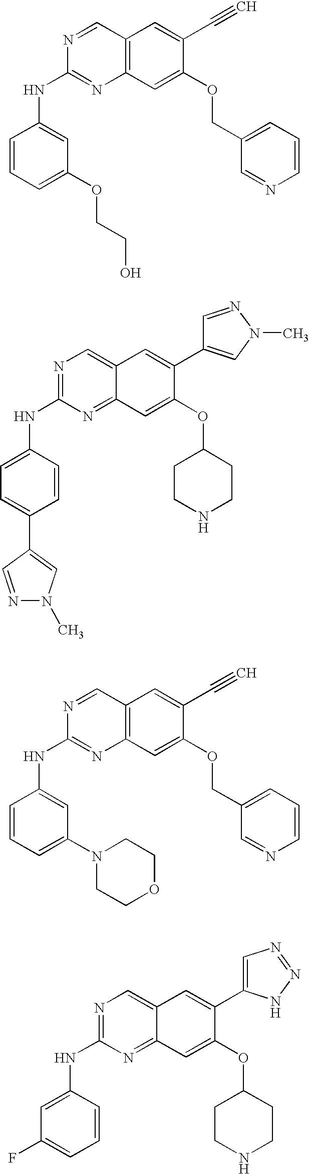

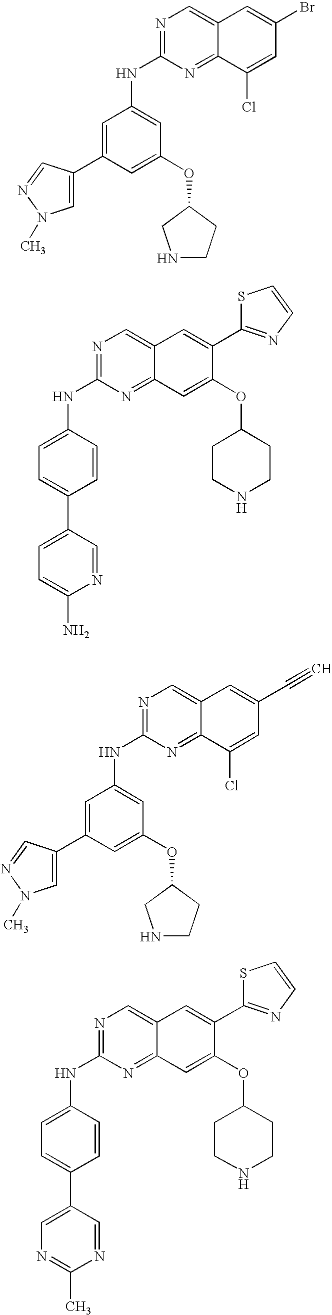

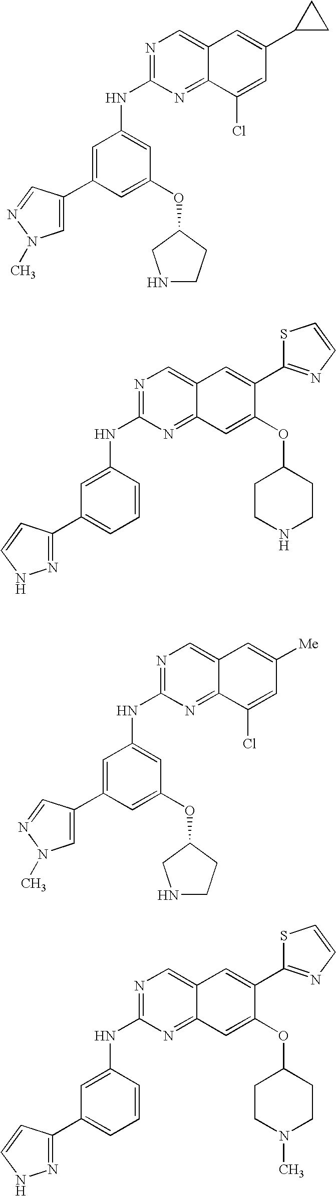

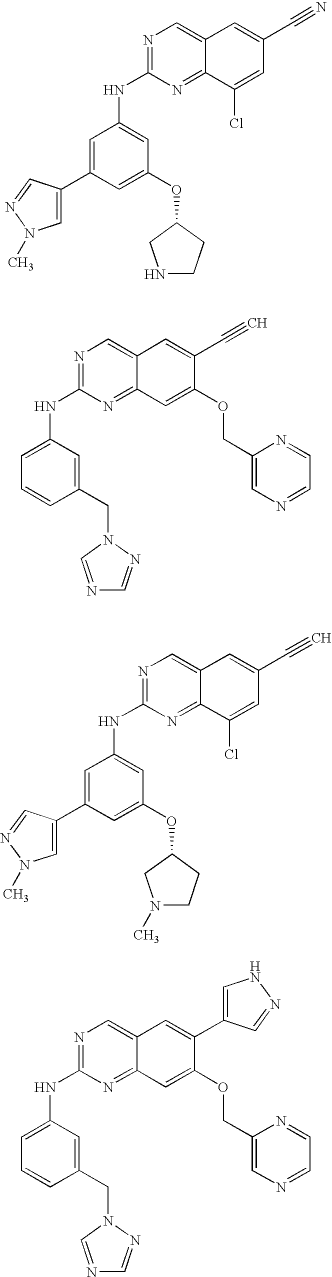

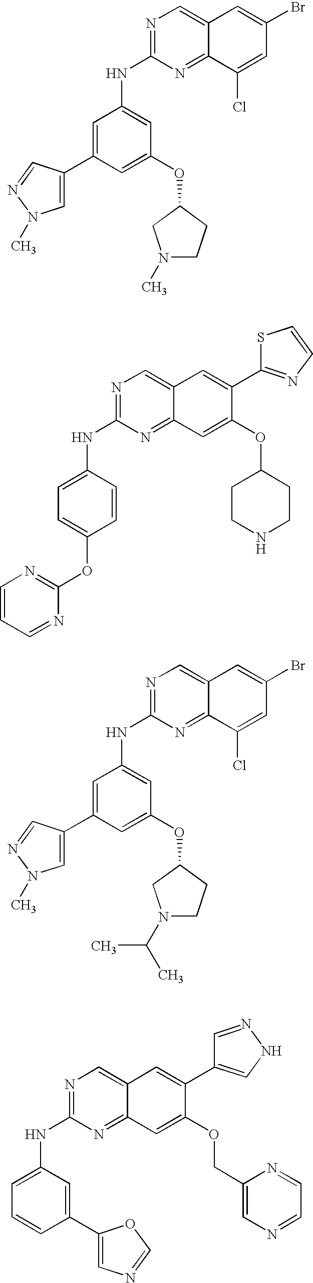

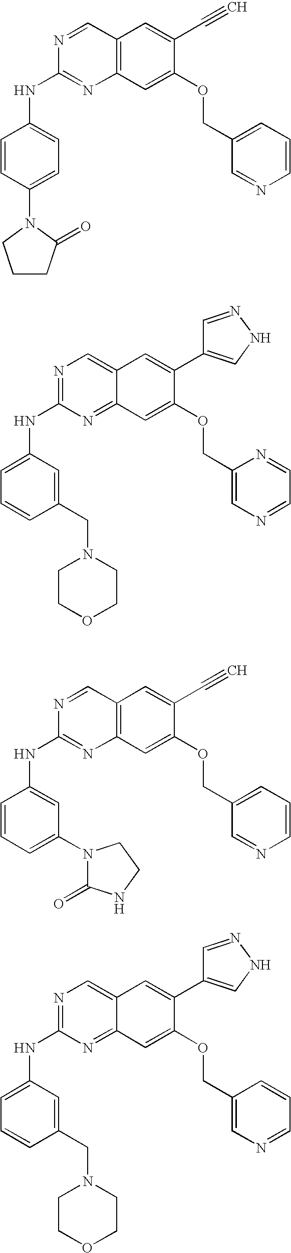

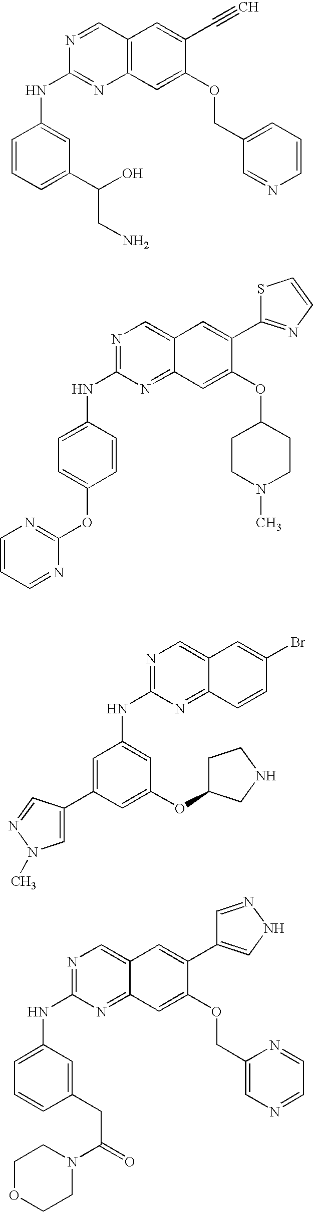

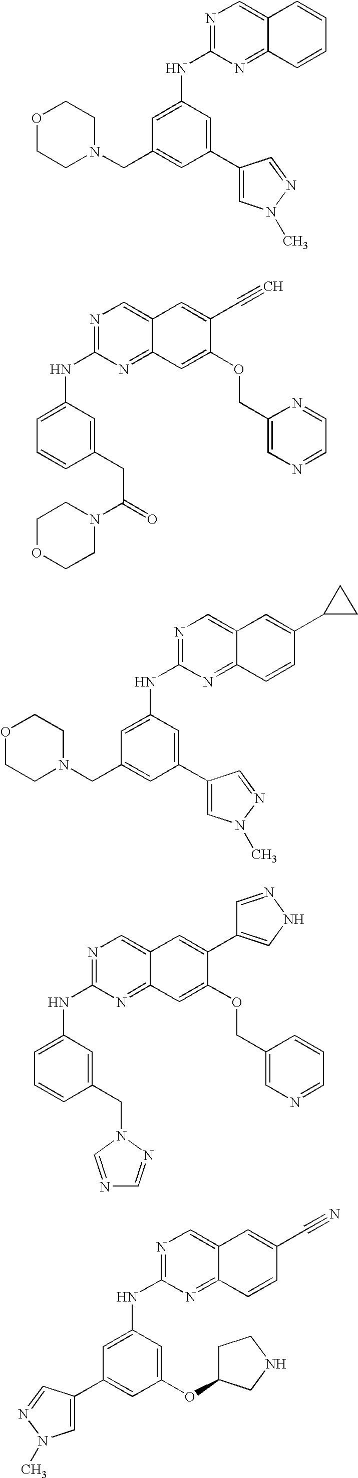

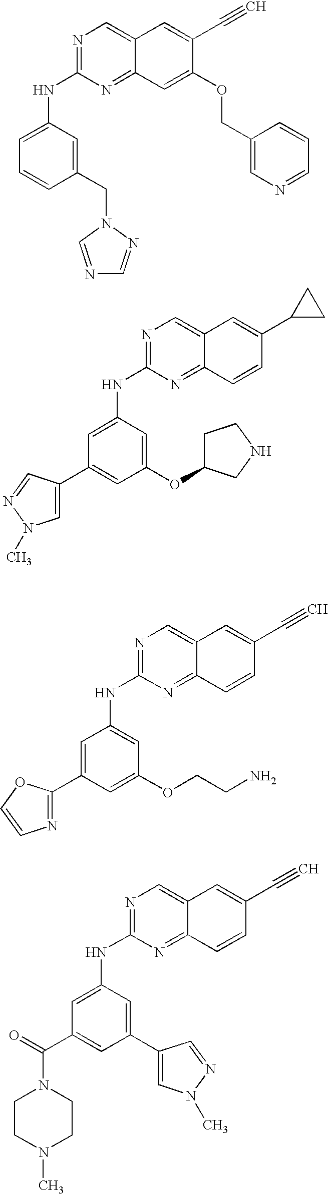

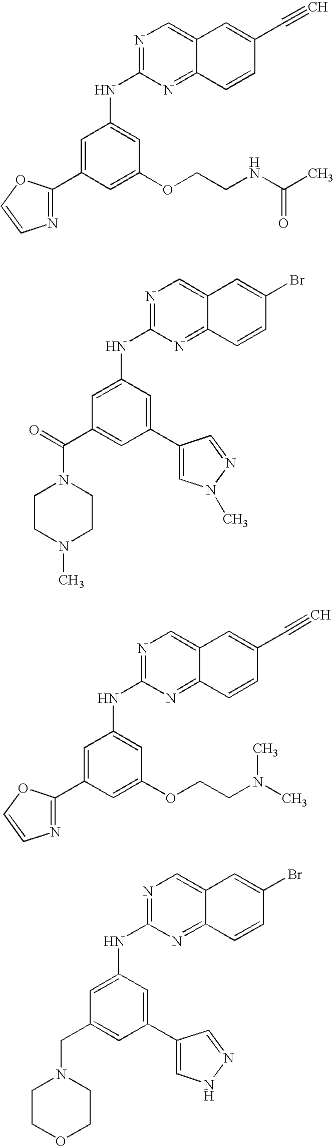

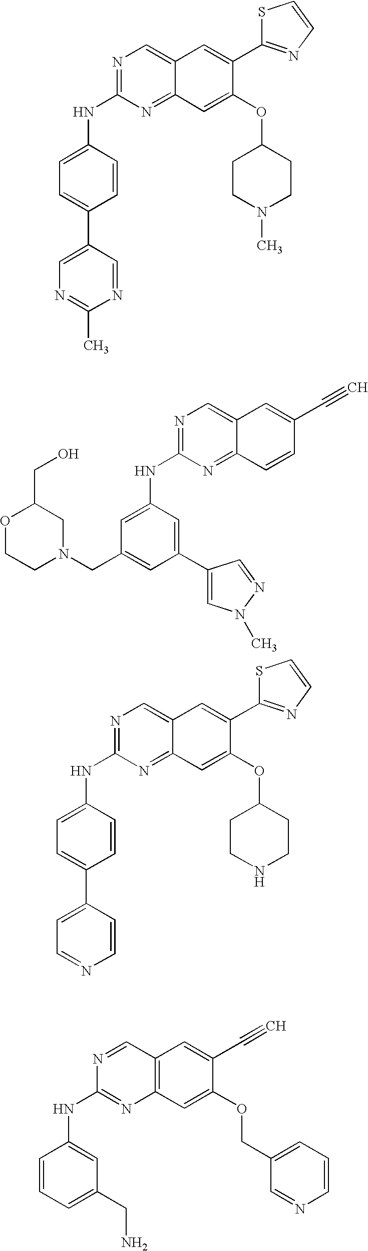

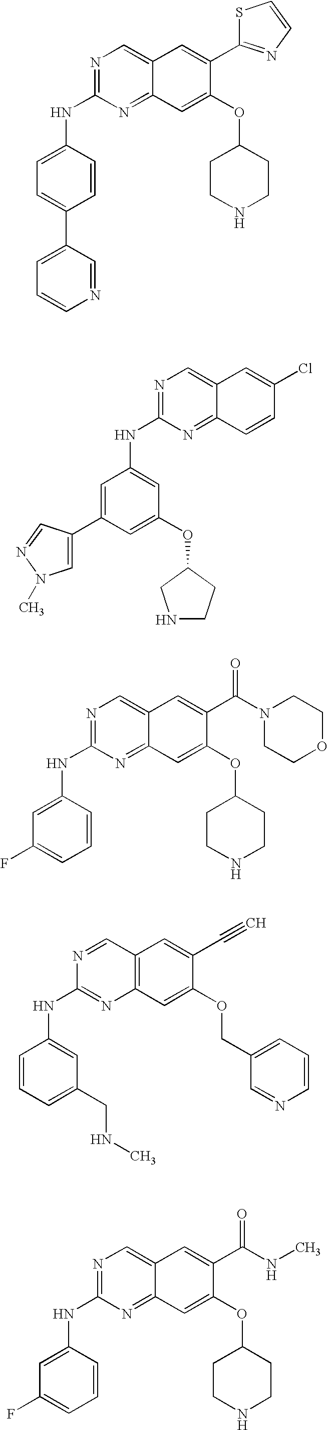

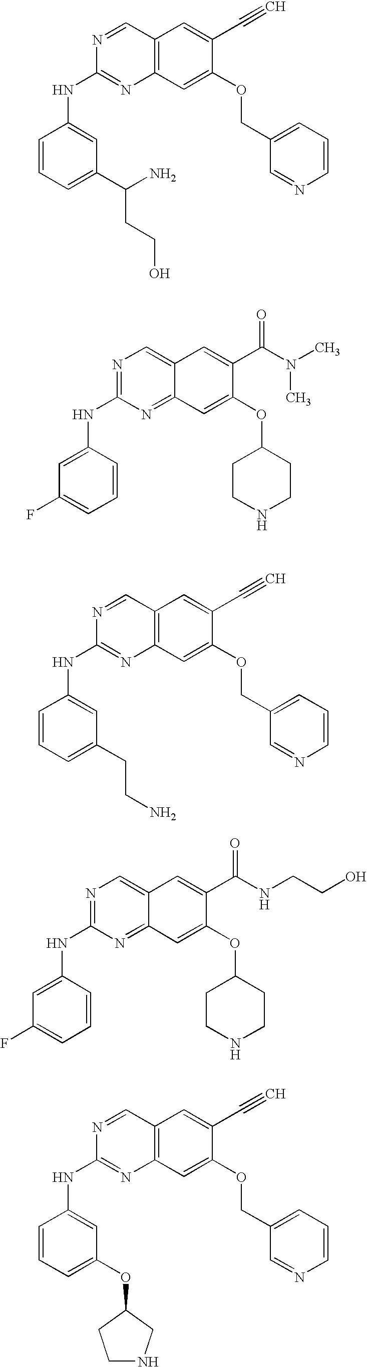

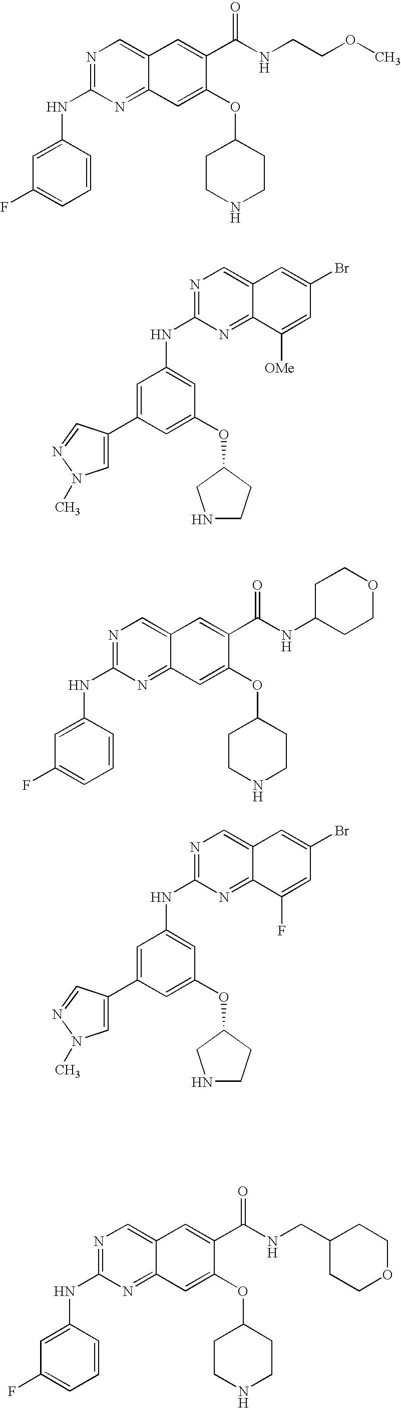

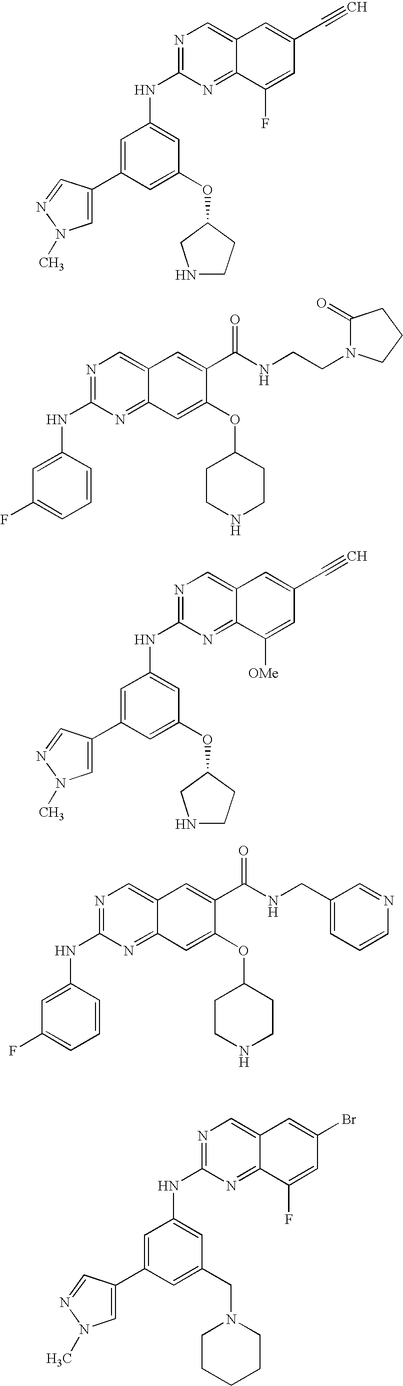

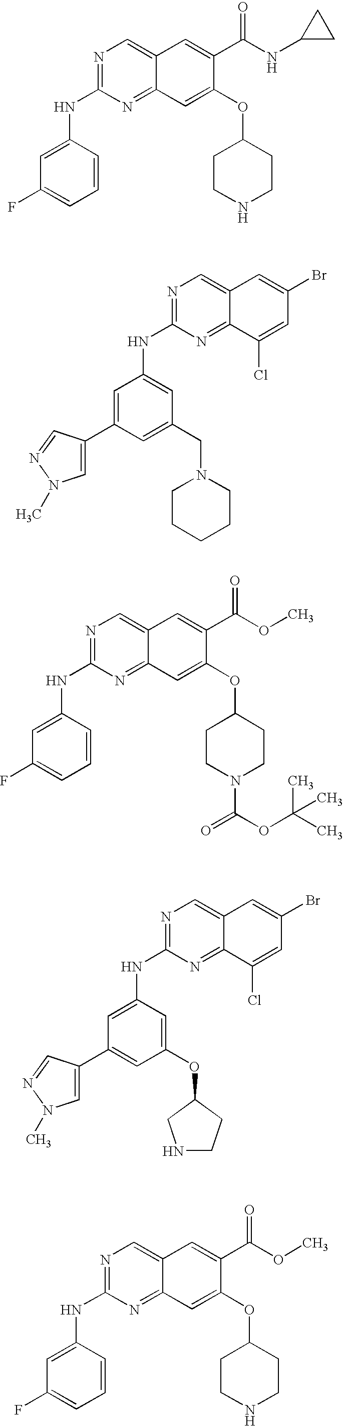

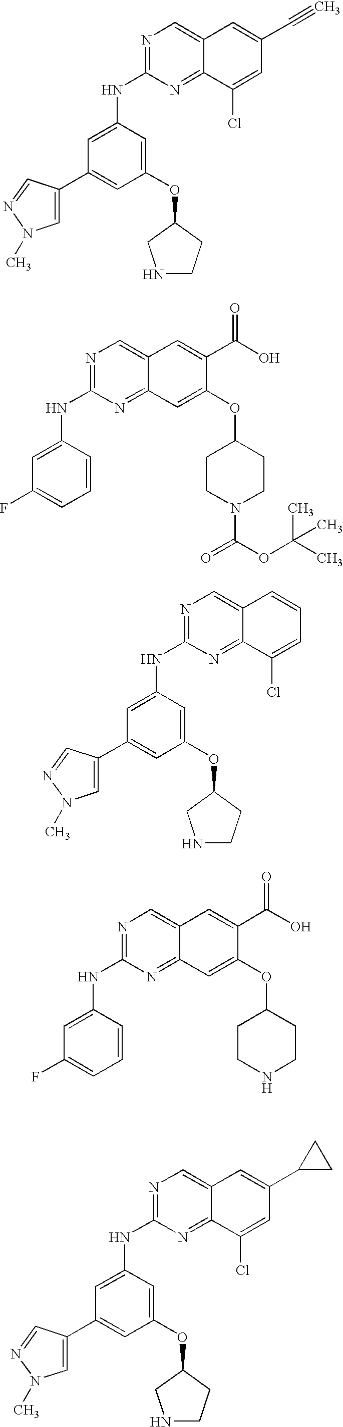

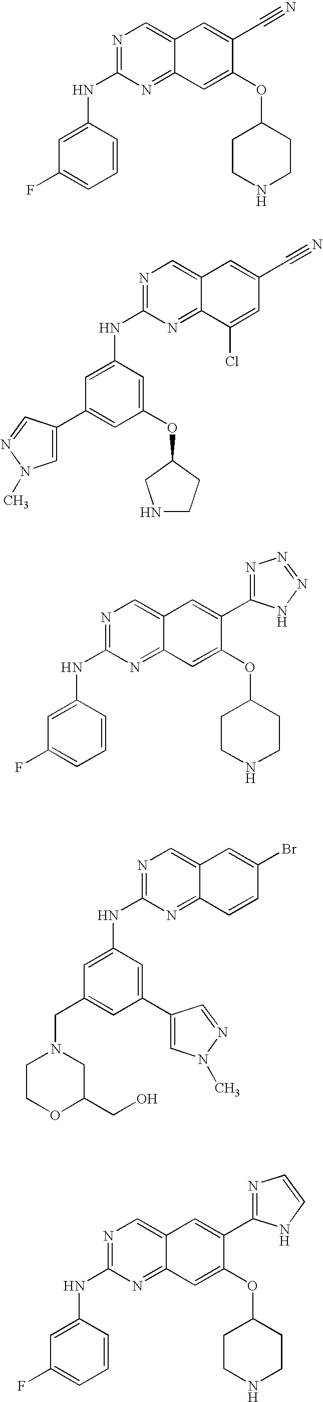

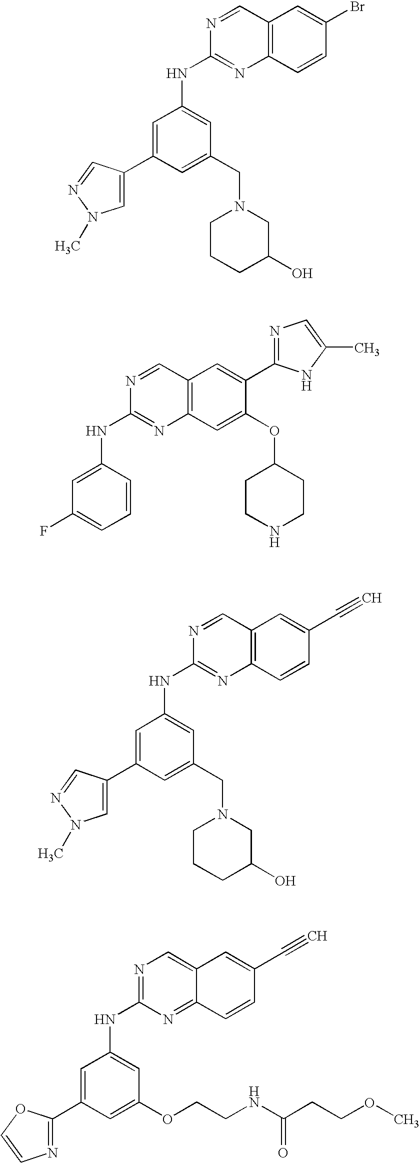

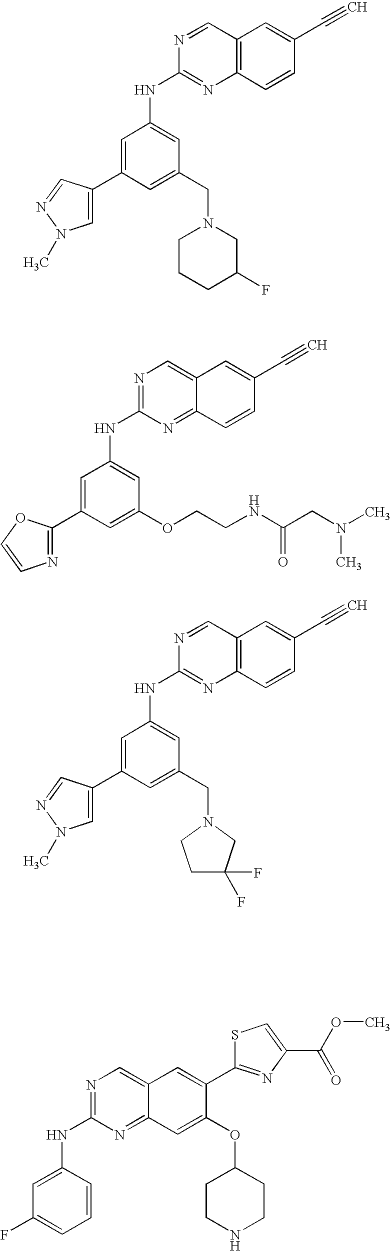

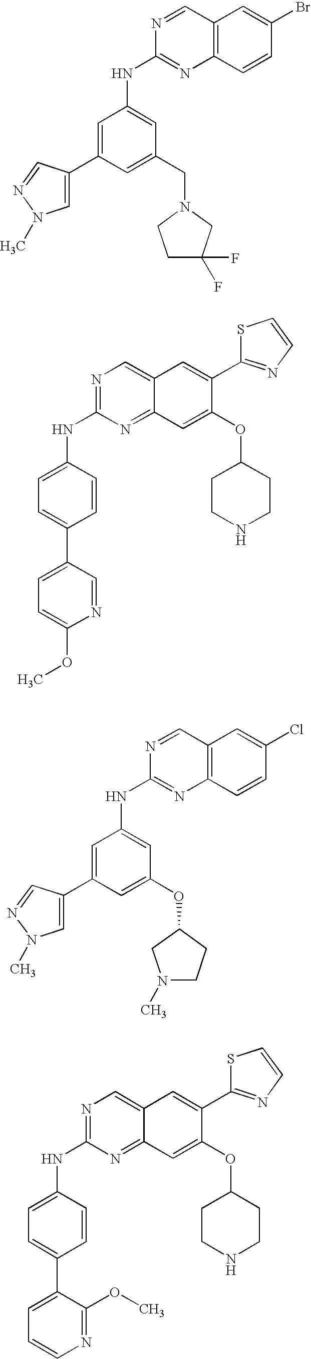

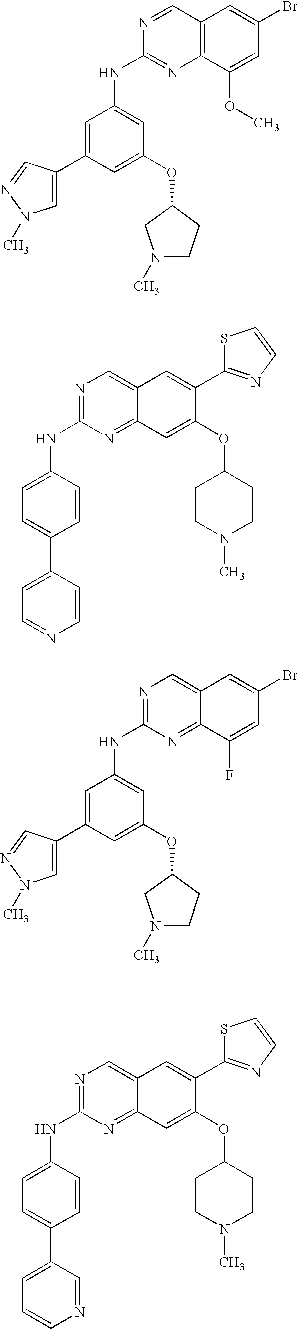

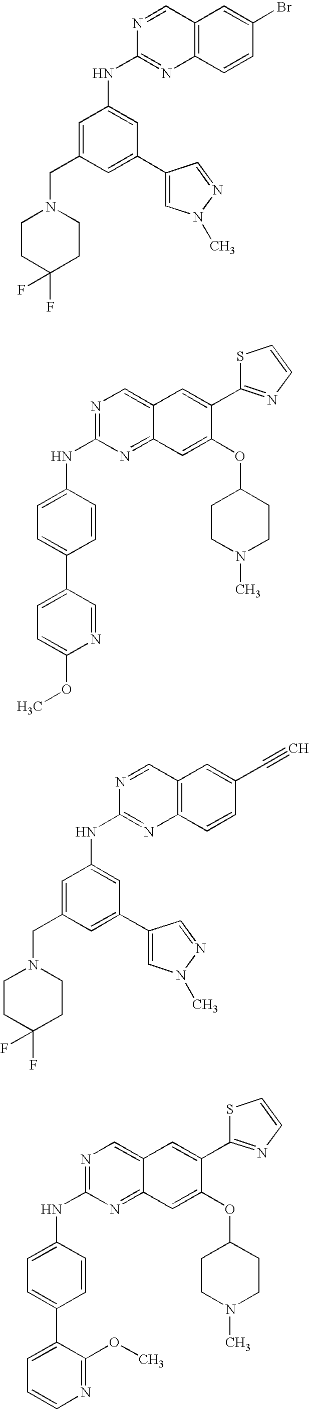

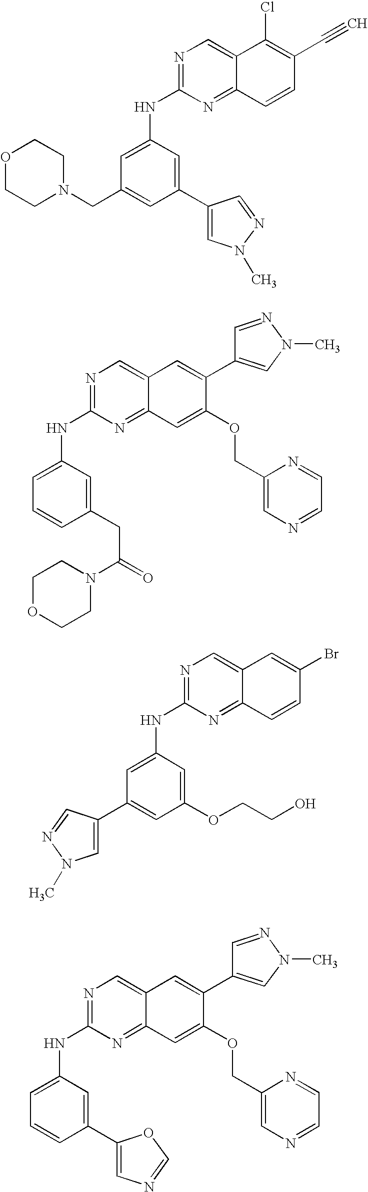

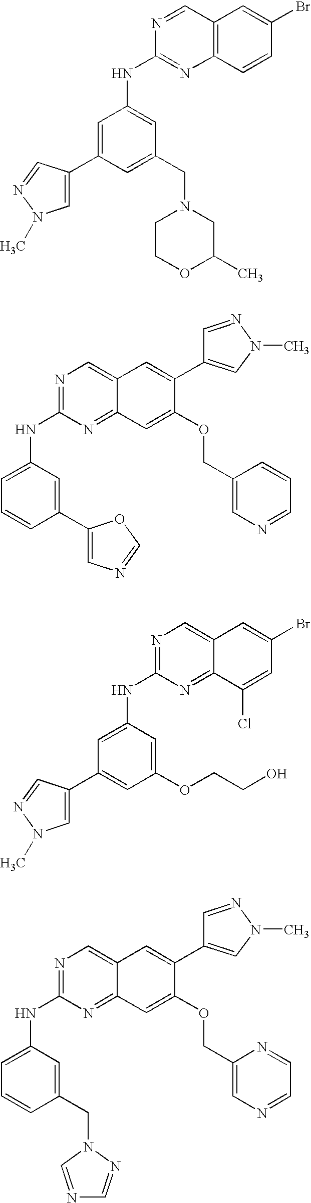

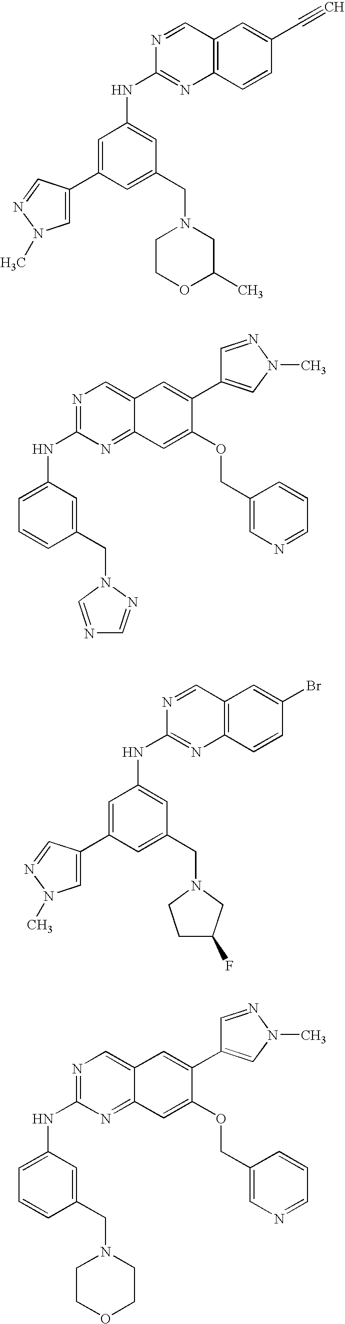

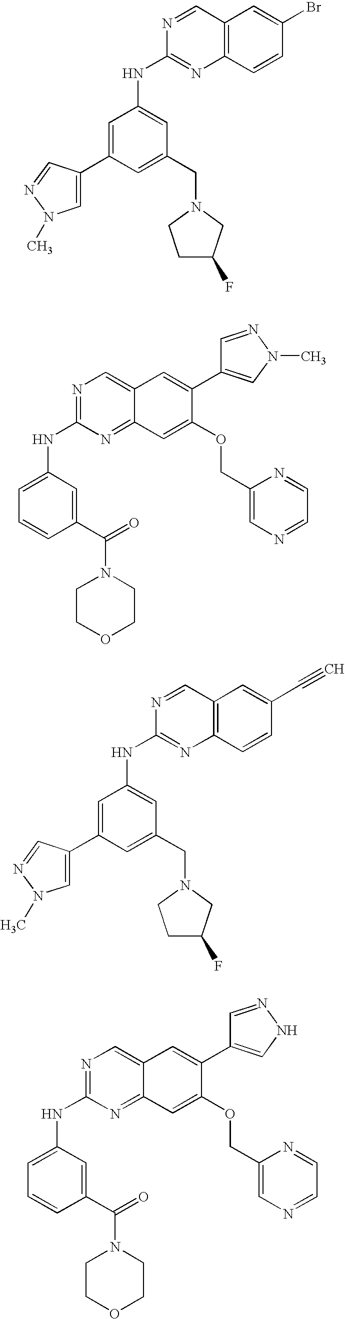

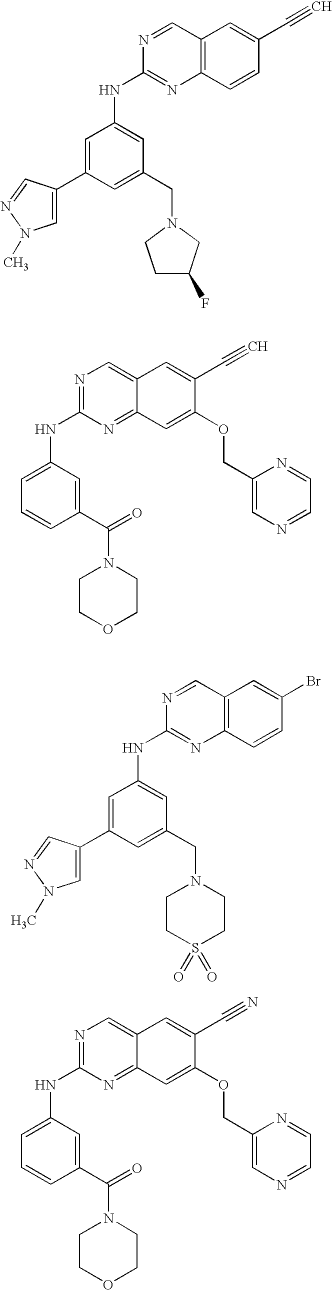

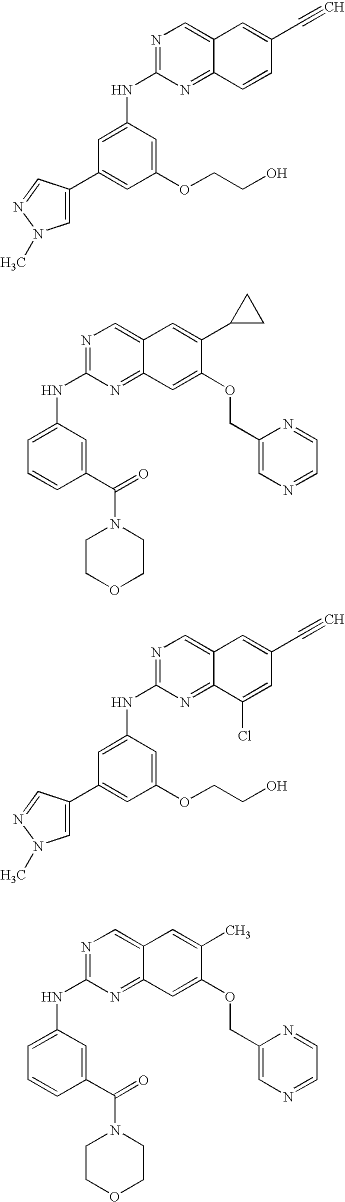

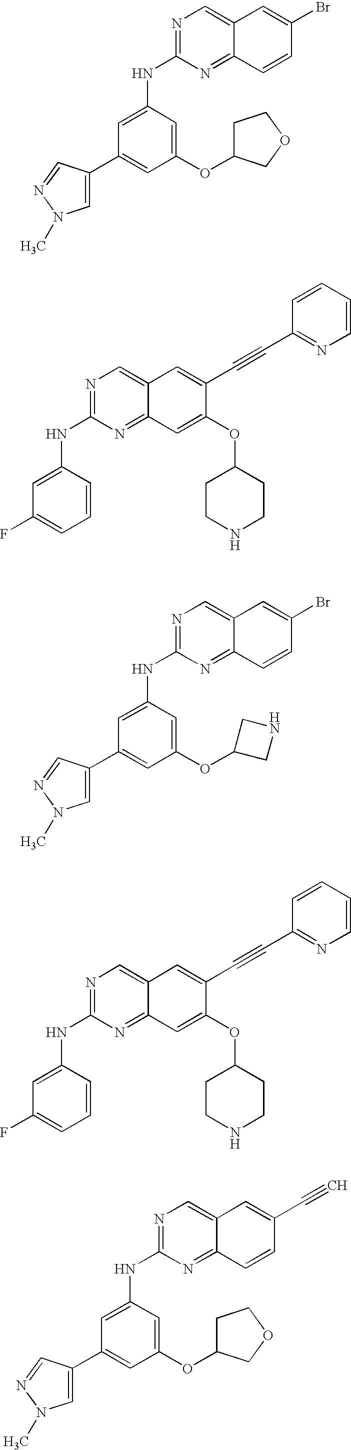

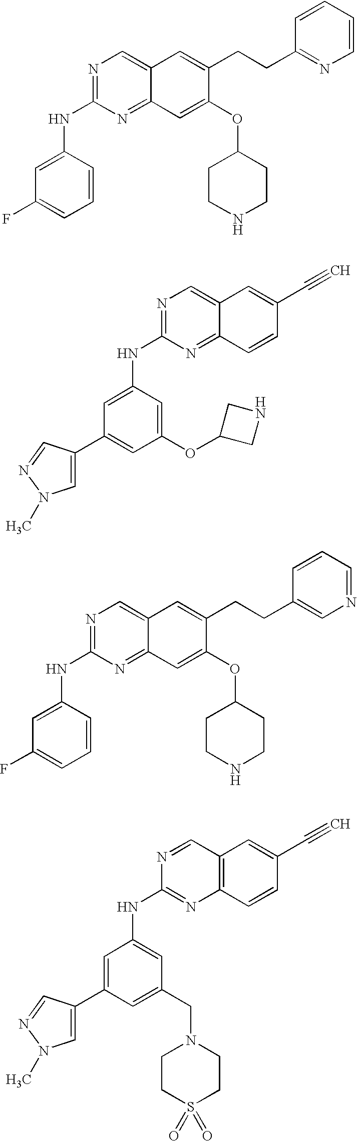

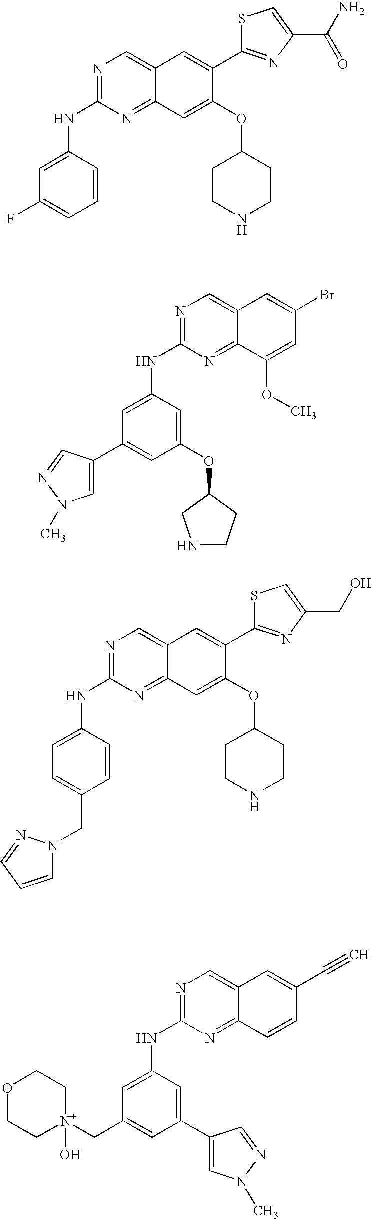

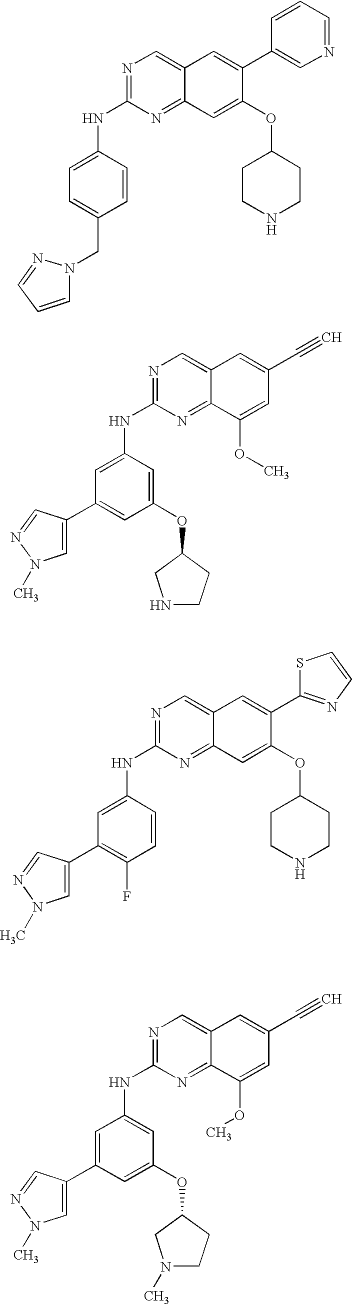

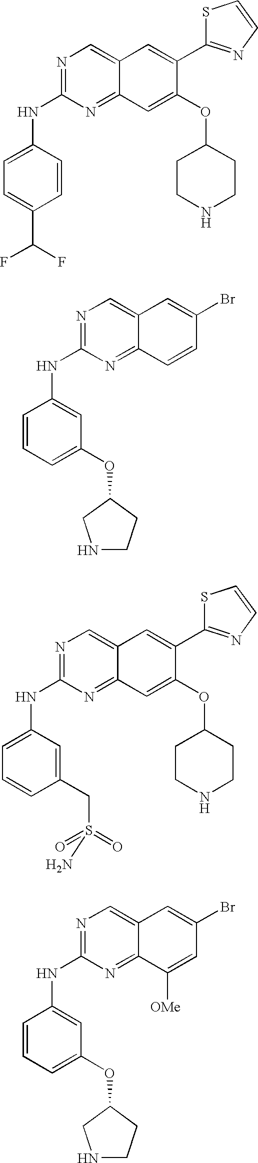

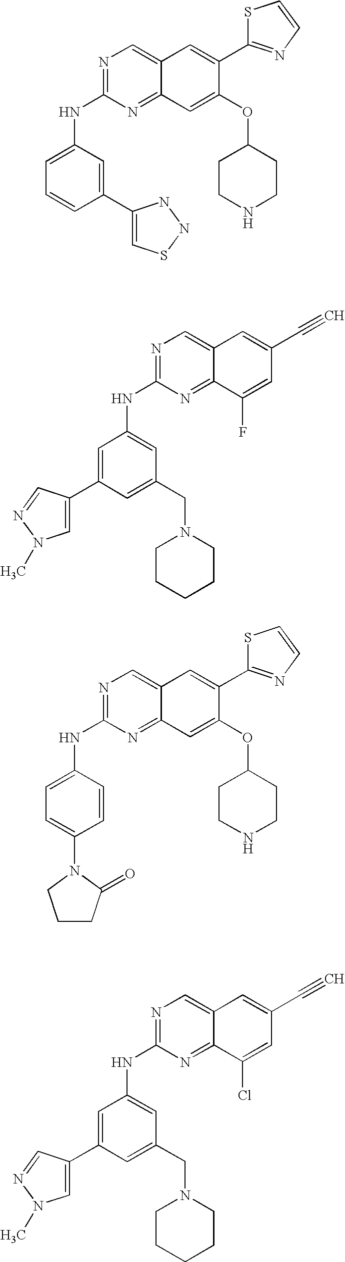

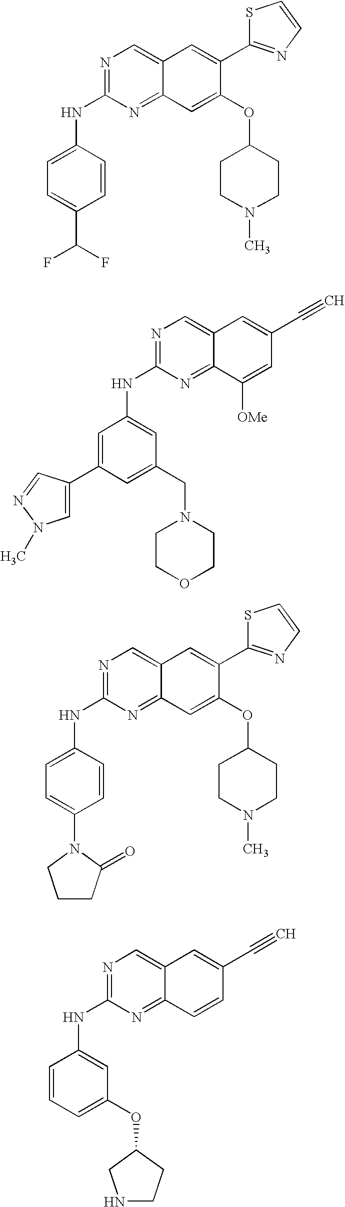

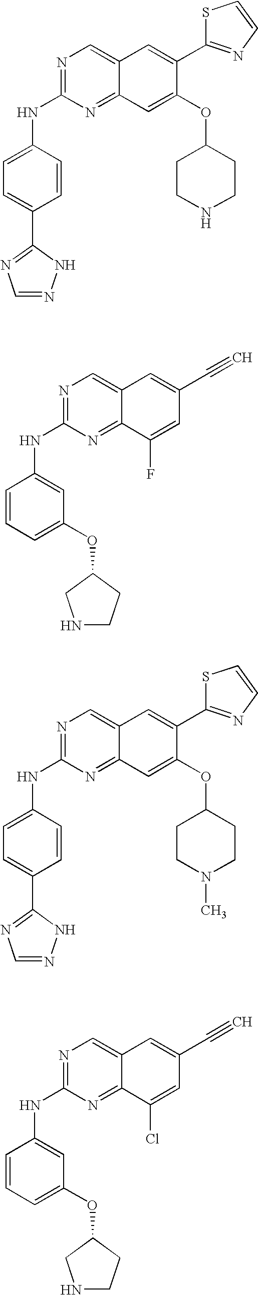

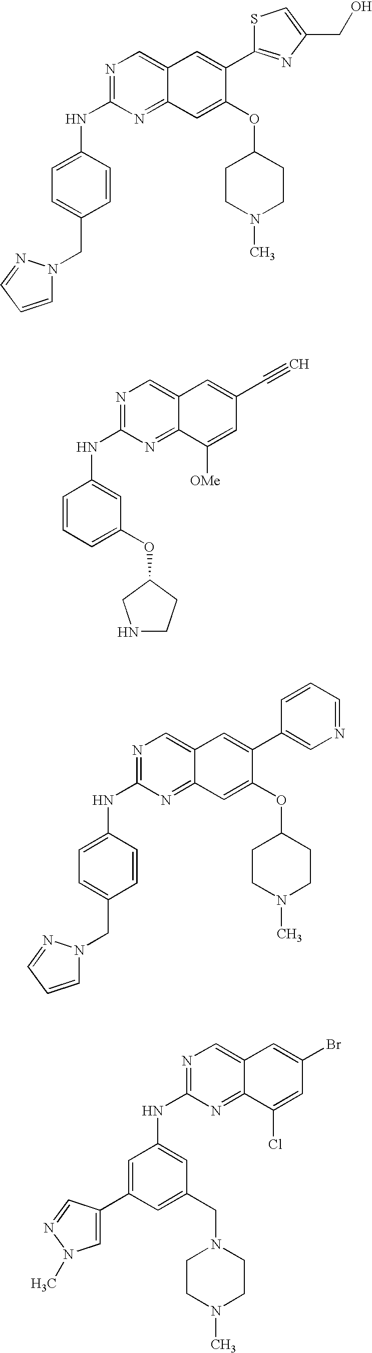

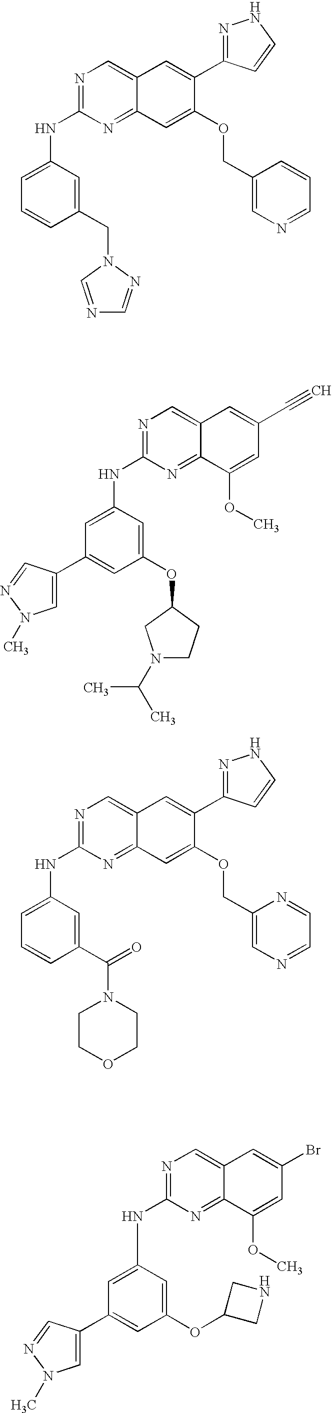

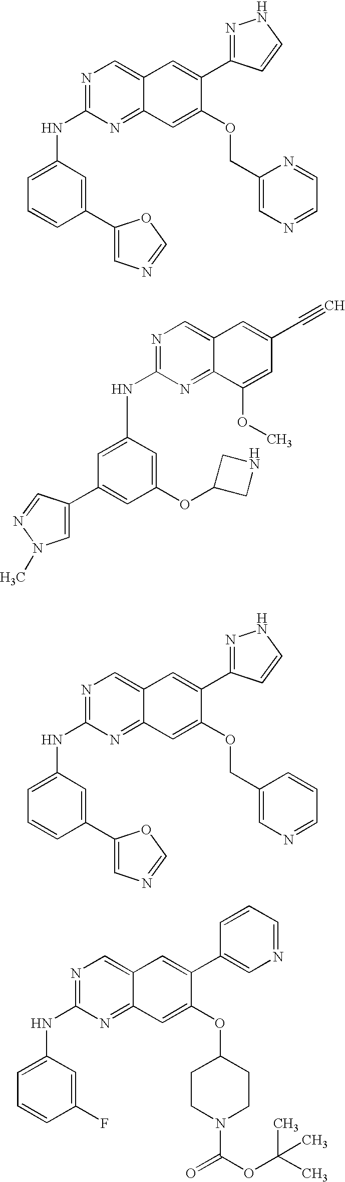

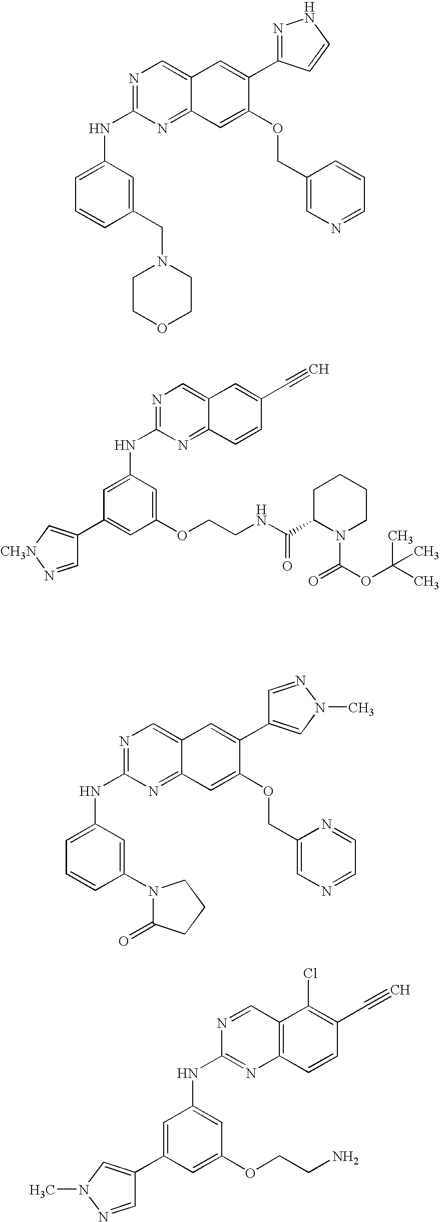

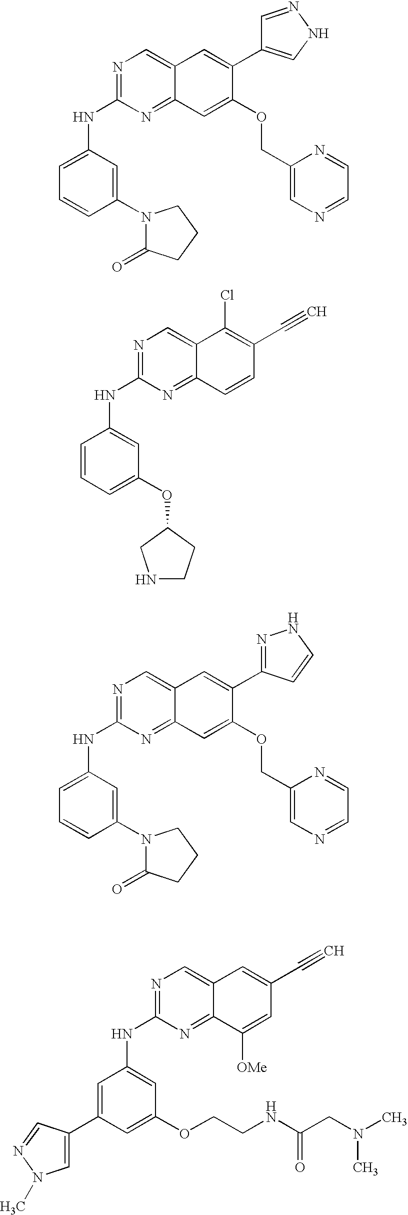

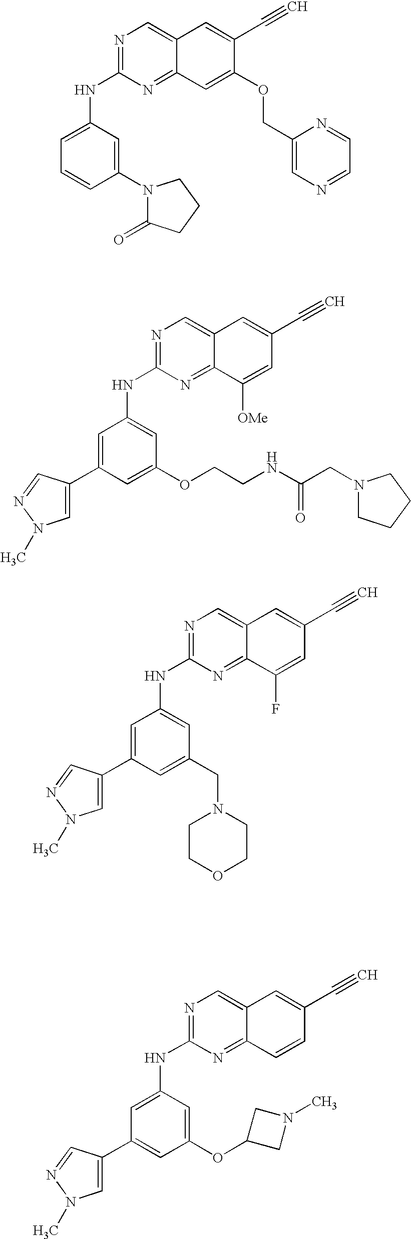

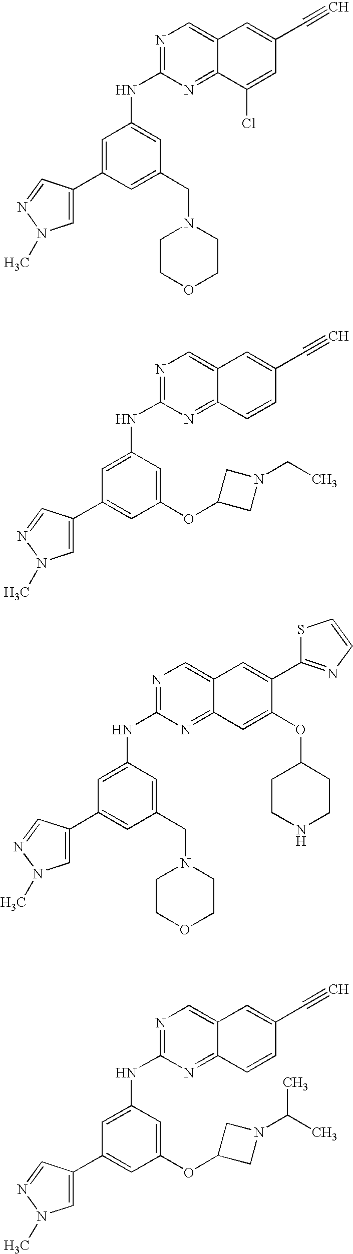

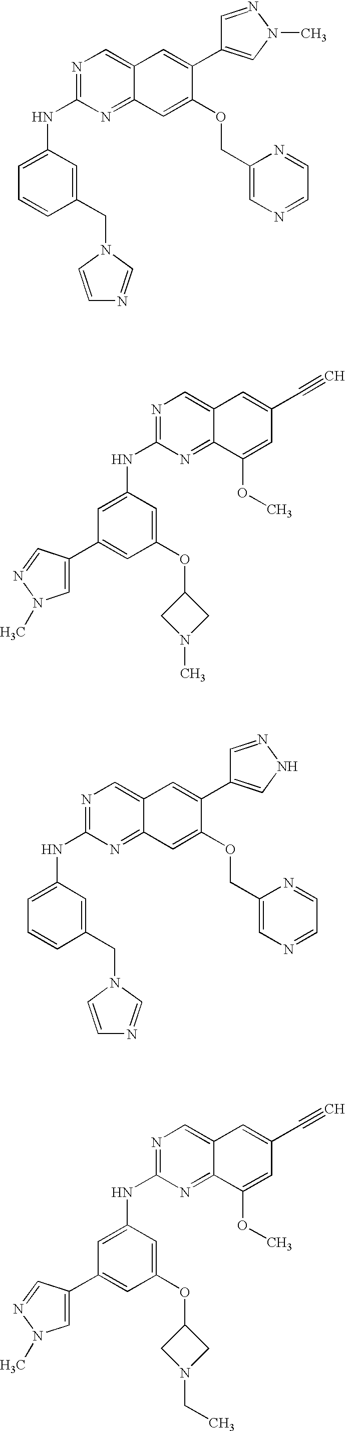

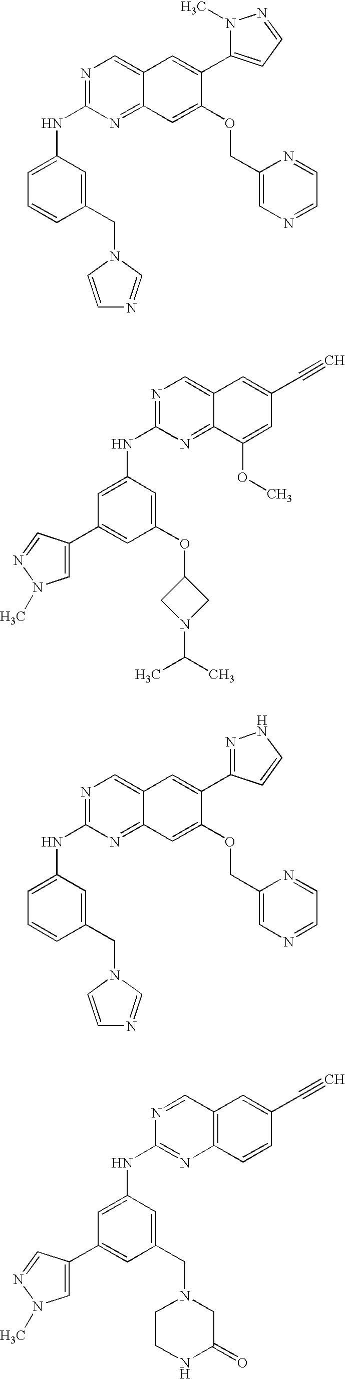

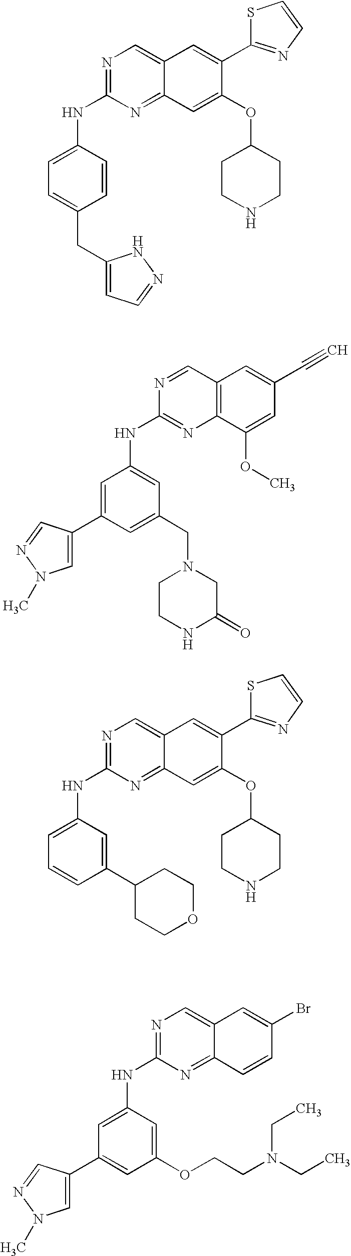

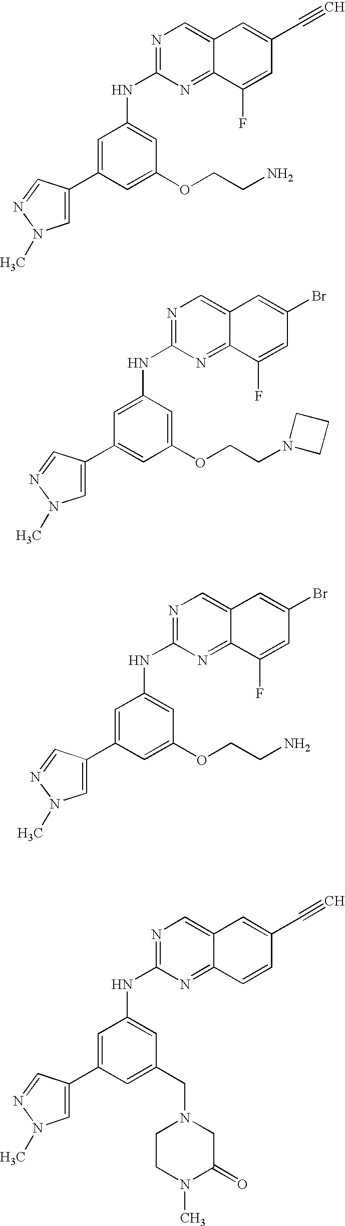

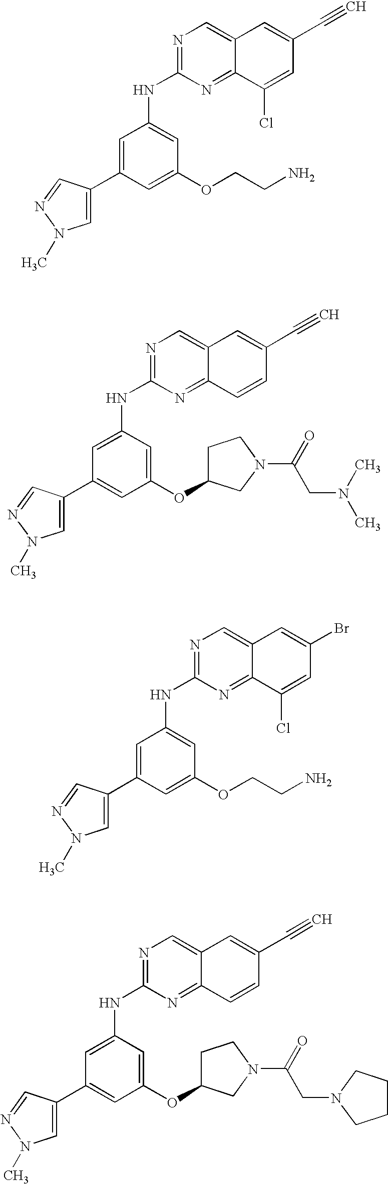

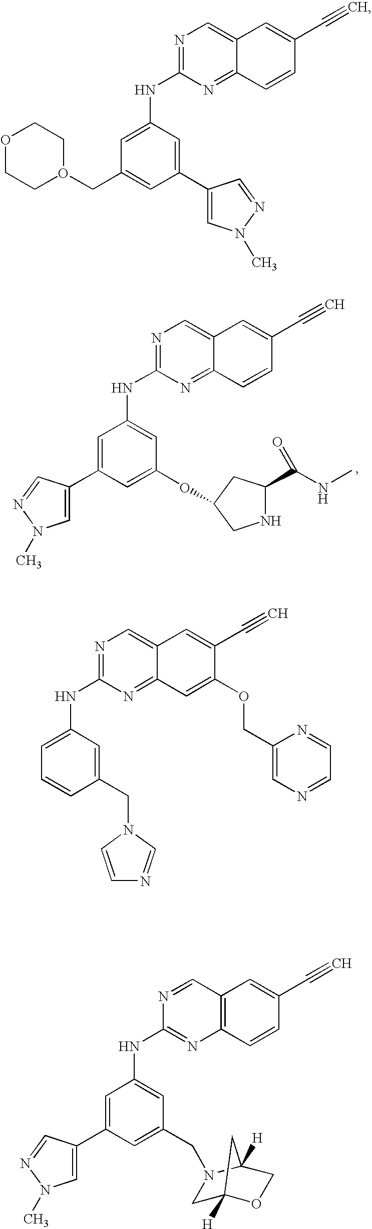

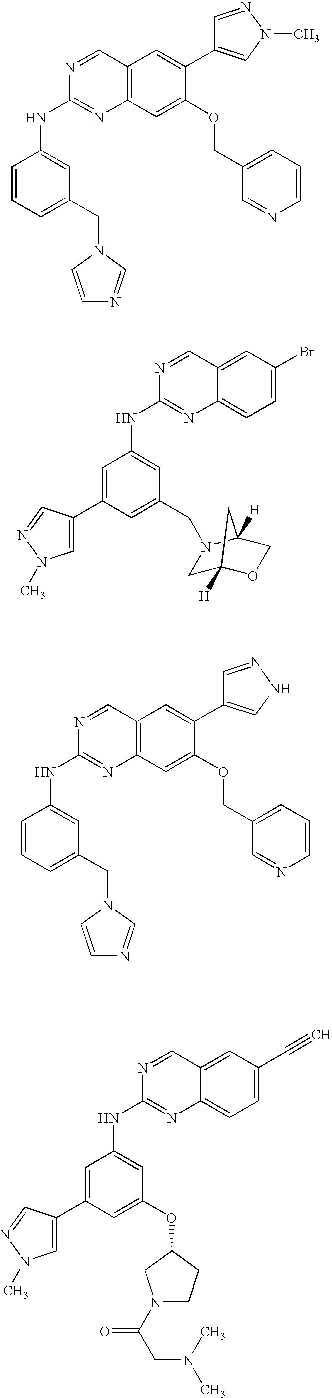

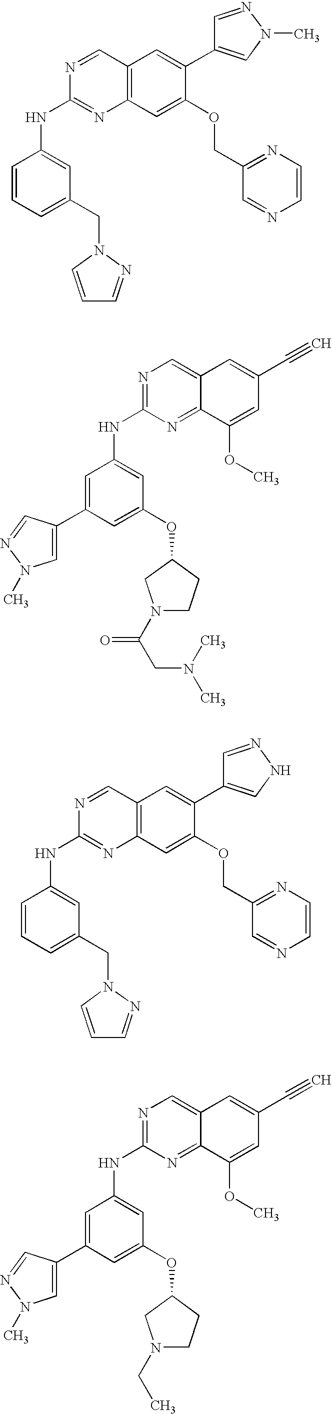

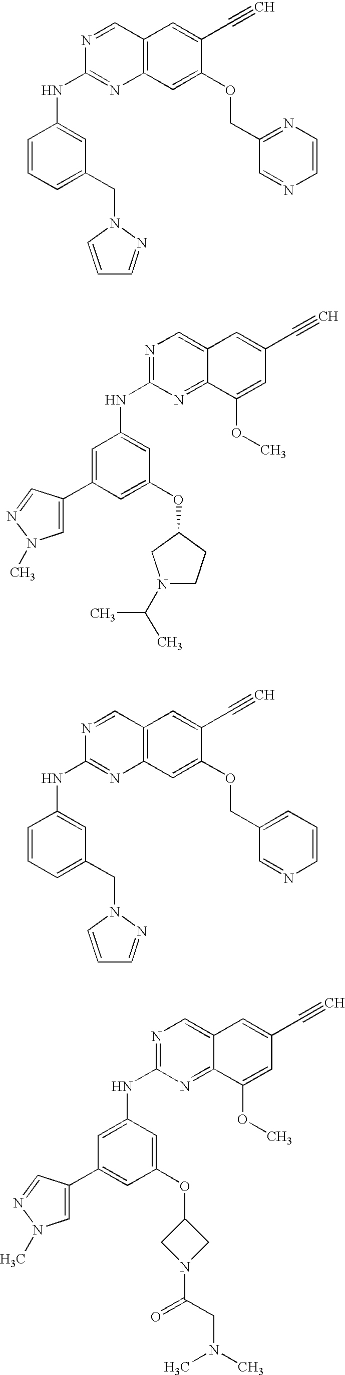

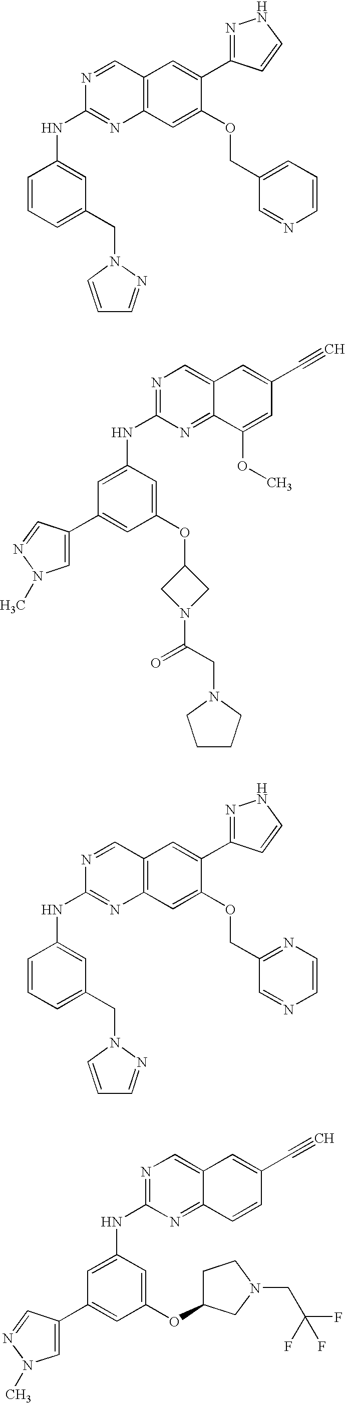

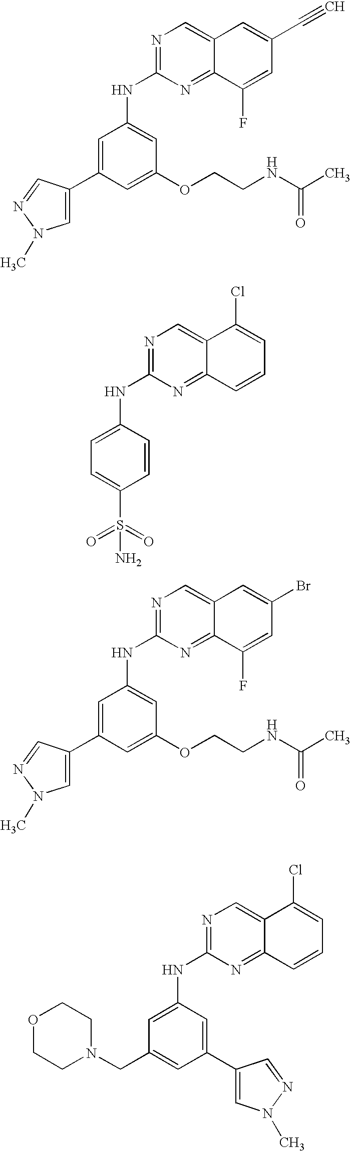

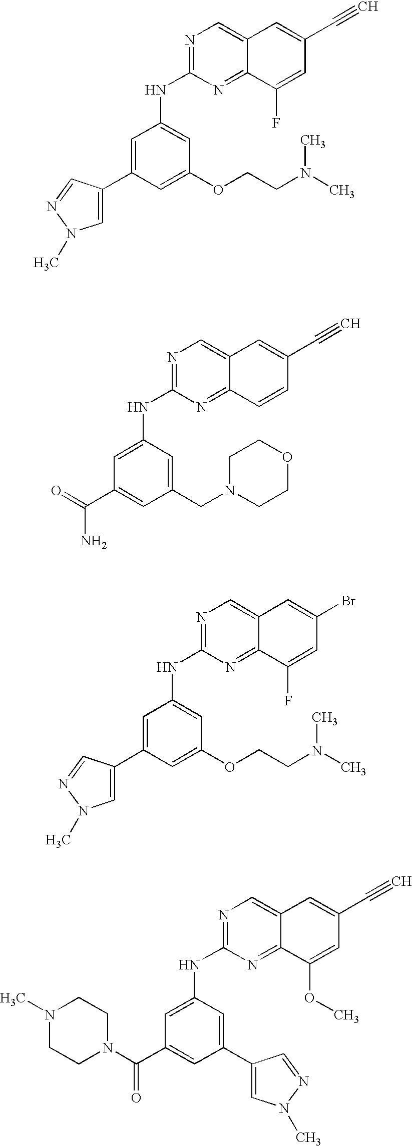

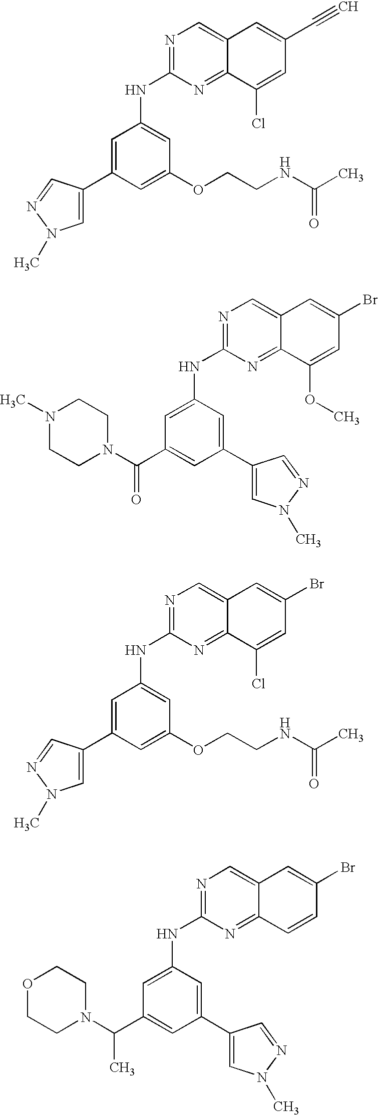

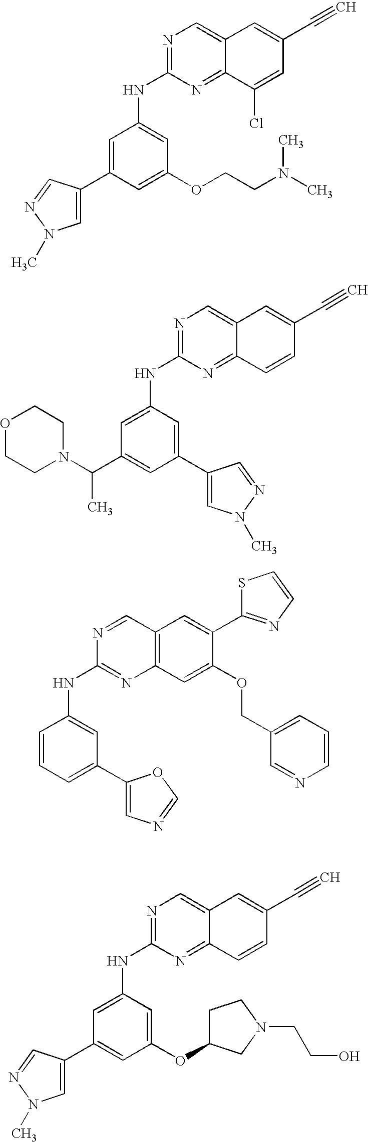

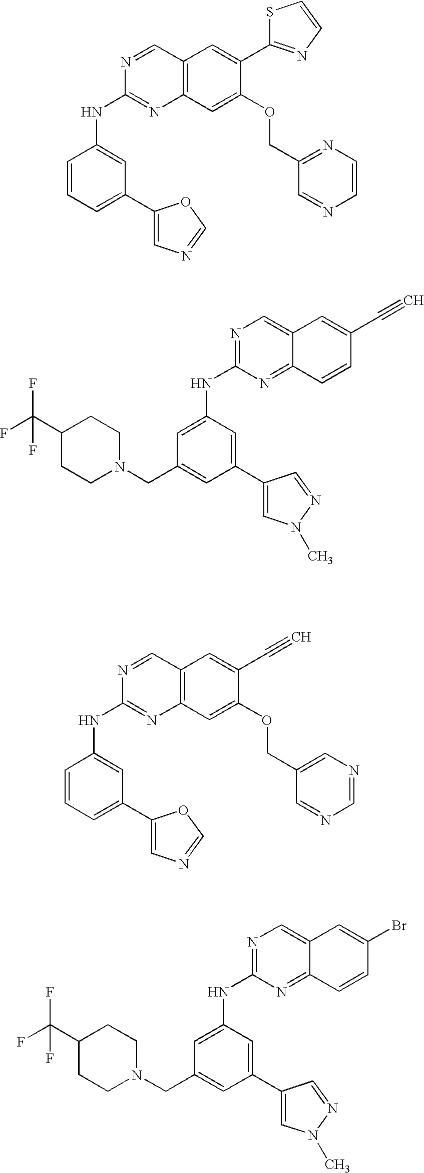

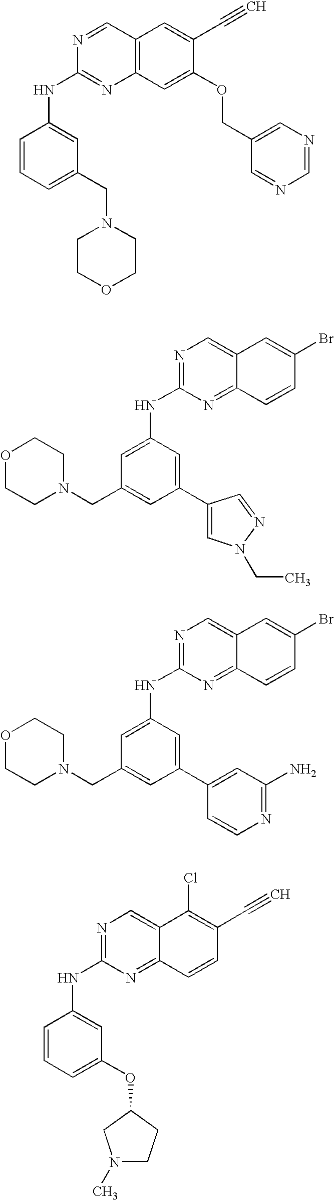

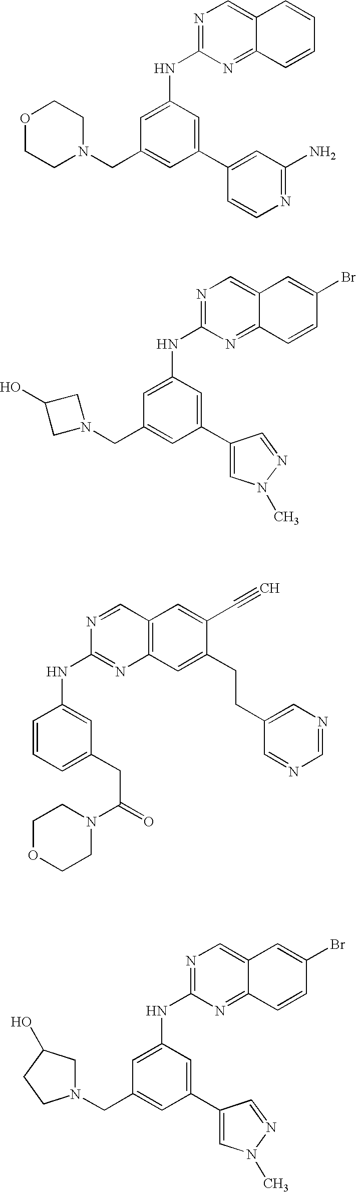

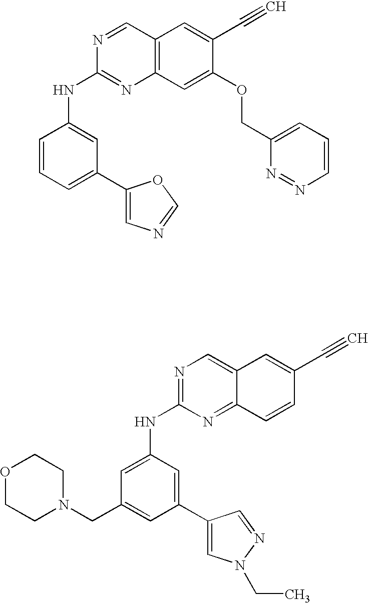

or pharmaceutically acceptable salt thereof.

In some embodiments, the invention provides compounds that have the Formula I:

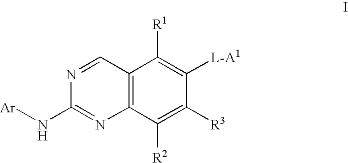

or a pharmaceutically acceptable salt thereof, wherein:

Ar is aryl, substituted aryl, heteroaryl, or substituted heteroaryl, including fused bicyclic systems;

R¹ is H, C₁₋₃ alkyl, halo, cyano, nitro, CF₃, imidazolyl, thiazolyl, oxazolyl, or amino;

R² and R³ are independently selected from the group consisting of H, alkoxy, substituted alkoxy, and halo;

L is a covalent bond, carbonyl, carbonylamino, aminocarbonyl, —O—, —S—, —SO—, —SO₂—, —NH—, C₁₋₃ alkyl, substituted C₁₋₃ alkyl, or an alkyl interrupted with —O—, —S—, —SO—, —SO₂—,

—NH—, carbonyl, carbonylamino, or aminocarbonyl; and

A¹ is alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, sulfonylamino, thioacyl, thiol, alkylthio, substituted alkylthio, aryl, substituted aryl, heteroaryl, substituted heteroaryl, cycloalkyl, substituted cycloalkyl, heterocyclyl, or substituted heterocyclyl.

According to some embodiments, when R¹, R², and R³ are each H and L is a covalent bond, then A¹ is other than aryl or substituted aryl.

According to some embodiments, when R¹, R², and R³ are each H, L is a covalent bond, and A¹ is Br, substituted phenyl, or substituted pyridinyl, then Ar is other than phenyl, phenyl substituted with piperazinyl or heterocyclylalkyloxy, or pyridinyl.

According to some embodiments, when R¹, R², and R³ are each H, L is a covalent bond, and A¹ is hydroxy or alkoxy, then Ar is other than phenyl substituted with one or more alkyl or halo.

According to some embodiments, when R¹, R², and R³ are each H and L is O, then A¹ is other than pyridinyl or substituted pyridinyl.

In some embodiments, the invention provides compounds that have the Formula I:

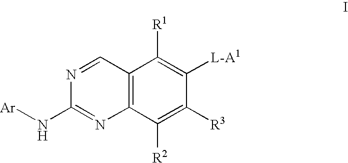

or a pharmaceutically acceptable salt thereof, wherein:

Ar is aryl, substituted aryl, heteroaryl, or substituted heteroaryl, including fused bicyclic systems;

R¹ is H, C₁₋₃ alkyl, halo, cyano, nitro, CF₃, imidazolyl, thiazolyl, oxazolyl, or amino;

R² is selected from the group consisting of H, alkoxy, substituted alkoxy, alkyl, substituted alkyl, CN, heteroaryloxy, substituted heteroaryloxy, heterocyclyloxy, substituted heterocyclyloxy, and halo;

R³ is selected from the group consisting of H, halo, CN, carboxy, alkyl, substituted alkyl, alkoxy, substituted alkoxy, aryloxy, substituted aryloxy, cycloalkyloxy, substituted cycloalkyloxy, heterocyclylalkyloxy, substituted heterocyclylalkyloxy, heteroaryloxy, substituted heteroaryloxy, heteroarylalkyloxy, substituted heteroarylalkyloxy, arylalkyloxy, substituted arylalkyloxy, heteroaryl, substituted heteroaryl, heterocyclyl, substituted heterocyclyl, heteroarylalkyl, substituted heteroarylalkyl, heterocyclylalkyl, substituted heterocyclylalkyl;

L is a covalent bond, carbonyl, carbonylamino, aminocarbonyl, —O—, —S—, —SO—, —SO₂—, —NH—, C₁₋₃ alkyl, substituted C₁₋₃ alkyl, C₂₋₃ alkenyl, C₂₋₃ alkynyl or an alkyl interrupted with —O—, —S—, —SO—, —SO₂—, —NH—, carbonyl, carbonylamino, or aminocarbonyl; and

A¹ is alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, sulfonylamino, thioacyl, thiol, alkylthio, substituted alkylthio, aryl, substituted aryl, heteroaryl, substituted heteroaryl, cycloalkyl, substituted cycloalkyl, heterocyclyl, or substituted heterocyclyl.

According to some embodiments, when R¹, R², and R³ are each H and L is a covalent bond, then A¹ is other than aryl or substituted aryl.

According to some embodiments, when R¹, R², and R³ are each H, L is a covalent bond, and A¹ is Br, substituted phenyl, or substituted pyridinyl, then Ar is other than phenyl, phenyl substituted with piperazinyl or heterocyclylalkyloxy, or pyridinyl.

According to some embodiments, when R¹, R², and R³ are each H, L is a covalent bond, and A¹ is hydroxy or alkoxy, then Ar is other than phenyl substituted with one or more alkyl or halo.

According to some embodiments, when R¹, R², and R³ are each H and L is O, then A¹ is other than pyridinyl or substituted pyridinyl.

According to some embodiments, L is a covalent bond. In some such embodiments, A¹ is an optionally substituted alkyne or an optionally substituted heterocyclyl or heteroaryl. Preferred alkynes include ethyne, 1-propyne, 3-hydroxypropyne, and 3-methoxypropyne, as well as other 3-alkoxypropynes. Preferred heteroaryls for these embodiments include thiazole, pyridine, imidazole, furan, 1,2,3-triazole, 1,2,4-triazole, pyrazole, isothiazole, oxazole, and isoxazole, each of which can be substituted. Specific heteroaryls for these embodiments include 2-thiazolyl; 5-hydroxymethyl-2-thiazolyl; 3-pyridyl, 5-methoxy-3-pyridyl; 6-amino-3-pyridyl; 4-thiazolyl; 3-pyrazolyl; and 4-pyrazolyl. Preferred heterocyclyl groups include pyrrolidine, morpholine, piperidine, and piperazine, each of which can be substituted.

Some specific embodiments include compounds wherein A¹ is selected from the following group:
OH, Br, methyl, ethyl, ethyne (—C≡CH), CN, CF₃, phenyl, COOH, COOMe, CONH₂,
1-hydroxy-1-methylethyl,
1-amino-1-methylethyl,
2-thiazolyl,
5-thiazolyl,
4-thiazolyl,
isoxazol-4-yl,
3-pyrazolyl,
4-pyrazolyl,
1-methyl-4-pyrazolyl,
1-methylpyrazol-5-yl,
2-furanyl, cyclopropyl,
4-hydroxymethyl-1,2-3-triazol-5-yl,
5-methoxypyridin-3-yl,
2-amino-3-methoxypyridin-5-yl,
3-methylpyridin-2-yl,
2-pyridyl,
3-pyridyl,
4-pyridyl,
4-methylpyridin-3-yl,
3-chloropyridin-4-yl,
1-morpholinyl,
1-pyrrolidinyl,
3-hydroxypyrrolidin-1-yl,
R-3-hydroxypyrrolidin-1-yl,
S-4-hydroxypiperidin-1-yl,
3-ketopiperazin-1-yl,
1-methylimidazol-2-yl,
5-methylthiazol-2-yl,
1-methylimidazol-5-yl,
3-hydroxy-1-propynyl,
3-methoxy-1-propynyl,
2-aminopyridin-4-yl,
3-methoxypyridin-5-yl,
2-aminopyridin-5-yl,
4-hydroxymethyl-1,2,3-triazol-5-yl,
2-amino-3-methoxypyridin-5-yl,
2-aminothiazol-5-yl,
1-(2-hydroxyethyl)pyrazol-4-yl,
1-(2-methoxyethyl)pyrazol-4-yl,
4-hydroxymethyl-thiazol-2-yl,
5-hydroxymethylthiazol-2-yl,
2-methoxypyrimidin-5-yl,
2-methoxypyridin5-yl,
2-hydroxyethylamino,
tetrahydropyran-4-yloxy,
isopropylamino,
2-pyridinylmethylamino,
2-methoxyethylamino,
3-pyridylmethylamino,
4-pyridylmethylamino,
2-pyridylamino, 3-pyridylamino,
2-(2-ketopyrrolidin-1-yl)ethylamino,
4-methylpiperazin-1-yl,
1-isopropylmethylpyrazol-4-yl,
methylamino,
1-methylpiperidin-4-ylamino,
4-isopropylpiperazin-1-yl,
isopropylamino,
pyrrolidin-1-yl,
cyclopropylamino,
2-fluoropyridin-3-yl,
2-(4-methylpiperazin-1-yl)pyridine-4-yl,
3-(benzoylamino)phenyl, and
2-amino-4-methoxypyrimidin-5-yl.

According to some embodiments, L is —O—.

According to some embodiments, L is —S—.

According to some embodiments, L is —SO₂—.

According to some embodiments, L is NH.

According to some embodiments, L is carbonyl.

According to some embodiments, L is aminocarbonyl or carbonylamino.

According to some embodiments, L is carbonyl amino.

According to some embodiments, L is aminocarbonyl.

According to some embodiments, L is an alkyl interrupted with —O—, —S—, —SO—, —SO₂—, —NH—, carbonyl, carbonylamino or aminocarbonyl.

According to some embodiments, L is —CH═CH— or —C≡C—.

According to some embodiments, A¹ is alkyl.

According to some embodiments, A¹ is substituted alkyl.

According to some embodiments, A¹ is alkenyl.

According to some embodiments, A¹ is substituted alkenyl.

According to some embodiments, A¹ is alkynyl.

According to some embodiments, A¹ is ethynyl, propynyl, phenylethynyl or pyridylethynyl.

According to some embodiments, A¹ is substituted alkynyl.

According to some embodiments, A¹ is alkoxy.

According to some embodiments, A¹ is substituted alkoxy.

According to some embodiments, A¹ is acyl.

According to some embodiments, A¹ is cyano.

According to some embodiments, A¹ is aryl.

According to some embodiments, A¹ is substituted aryl.

According to some embodiments, A¹ is substituted phenyl.

According to some embodiments, A¹ is heteroaryl.

According to some embodiments, A¹ is substituted heteroaryl.

According to some embodiments, the heteroaryl or substituted heteroaryl is selected from the group consisting of pyridyl, pyrazolyl, thiazolyl, pyrimidyl, pyridazinyl, oxazolyl, isoxazolyl, substituted pyridyl, substituted pyrazolyl, substituted thiazolyl, substituted pyrimidyl, substituted pyridazinyl, substituted oxazolyl and substituted isoxazolyl.

According to some embodiments, A¹ is cycloalkyl.

According to some embodiments, A¹ is substituted cycloalkyl.

According to some embodiments, A¹ is heterocyclyl.

According to some embodiments, A¹ is substituted heterocyclyl.

According to some embodiments, the heterocyclyl or substituted heterocyclyl is selected from the group consisting of piperidinyl, piperazinyl, pyrrolidinyl, tetrahydrofuranyl, tetrahydrothiophenyl, morpholinyl, thiomorpholino, substituted piperidinyl, substituted piperazinyl, substituted pyrrolidinyl, substituted tetrahydrofuranyl, substituted tetrahydrothiophenyl, substituted morpholinyl and substituted thiomorpholino.

According to some embodiments, A¹ is hydroxy.

According to some embodiments, A¹ is halo.

According to some embodiments, A¹ is cyano.

In some embodiments, -L-A¹ is —Br, —C≡CH, —C≡N, 2-thiazolyl, or 1-methylimidazol-2-yl.

According to some embodiments, R¹ is H, C₁₋₃ alkyl, halo, cyano, nitro, CF₃ or amino.

According to some embodiments, R¹ is H, C₁₋₃ alkyl, halo, cyano, nitro or amino.

According to some embodiments, R¹ is H, C₁₋₃ alkyl, halo, cyano, imidazolyl, thiazolyl, oxazolyl or amino.

According to some embodiments, R¹ is H, C₁₋₃ alkyl, halo or cyano.

According to some embodiments, R¹ is H, C₁₋₃alkyl, or halo.

According to some embodiments, R¹ is H or halo.

According to some embodiments, R¹ is H.

According to some embodiments, R¹ is halo.

According to some embodiments, R² and R³ are independently selected from the group consisting of H, alkoxy, substituted alkoxy, and halo;

According to some embodiments, R² and R³ are independently selected from the group consisting of H, halo and alkoxy.

According to some embodiments, R² and R³ are independently selected from the group consisting of H, and halo.

According to some embodiments, R² and R³ are independently selected from the group consisting of H and alkoxy.

According to some embodiments, R² and R³ are independently selected from the group consisting of H and C₁₋₆ alkoxy.

According to some embodiments, R² and R³ are independently selected from the group consisting of H and methoxy.

According to some embodiments, at least one of R² and R³ is H.

According to some embodiments, both R² and R³ are H.

According to some embodiments, R² is H.

According to some embodiments, R³ is H.

In some embodiments, one of R² and R³ is H and the other of R² and R³ is alkoxy, substituted alkoxy, aryloxy, substituted aryloxy, cycloalkyloxy, substituted cycloalkyloxy, heterocyclylalkyloxy, substituted heterocyclylalkyloxy, heteroaryloxy, substituted heteroaryloxy, heteroarylalkyloxy, substituted heteroarylalkyloxy, arylalkyloxy, or substituted arylalkyloxy.

In some embodiments, one of R² and R³ is H and the other is arylalkoxy, alkoxy or substituted alkoxy, or a substituted or unsubstituted heteroaryloxy, heteroarylalkyloxy, heterocyclyloxy, or heterocyclylalkyloxy. In such embodiments, R² is often H and R³ is substituted or unsubstituted alkoxy or heterocyclyloxy group.

In certain embodiments, R² is selected from H, F, Cl, Br, CN, CF₃, methoxy, ethoxy, isopropoxy, 4-piperidinyloxy, 3-azetidinyloxy, and 2-aminoethoxy.

In some embodiments, R³ is selected from:
H, C₁, CF₃, CN, COOH,
isopropoxy, methoxy, cyclopentyloxy,
2-aminoethoxy,
4-piperidinyloxy,
1-isopropyl-piperidin-4-yl,
4-piperidinylmethoxy,
1-methylpiperidin-3-ylmethoxy,
2-(4-piperidinyl)-ethoxy,
2-(1-methyl-4-piperidinyl)-ethoxy,
(1-methyl-4-piperidinyl)methoxy,
2-thiazolylmethoxy,
3-pyridylmethoxy,
4-pyridylmethoxy,
1-(4-pyridyl)-1-ethoxy,
2-pyridylmethoxy,
5-thiazolylmethoxy,
2-(4-piperidinyl)-ethoxy,
1-methyl-4-piperidinyloxy,
cyclopentyloxy,
2-(4-morpholinyl)-ethoxy,
2-methoxyethoxy,
1-aminocyclopropylmethoxy,
1-N-acetylaminocycloprop-1-ylmethoxy,
aminocarbonylmethoxy,
N-methylaminocarbonylmethoxy,
3-pyrrolidinyloxy,
R-3-pyrrolidinyloxy,
S-3-pyrrolidinyloxy,
R-(1-methylpyrrolidin-3-yl)oxy,
S-(1-methylpyrrolidin-3-yl)oxy,
pyrrolidiny-3-ylmethoxy,
piperidin-3-ylmethoxy,
R-piperidin-3-ylmethoxy,
S-piperidin-3-ylmethoxy,
2-(4-methyl-1-piperazinyl)ethyl,
1-methylpyrrolidin-3-ylmethoxy,
R-(1-methylpiperidin-3-ylmethoxy,
S-(1-methylpiperidin-3-ylmethoxy,
(3-chloro-4-pyridyl)methoxy,
2-methoxypyridyn-6-ylmethoxy,
(5-methylisoxal-3-ylmethoxy,
5-thiazolylmethoxy,
(3-fluorophenyl)methoxy,
(3-methoxyphenyl)methoxy,
phenylmethoxy,
(3-cyanophenyl)methoxy,
3-fluorophenylmethoxy,
3-methoxyphenylmethoxy,
2-pyrazinylmethoxy,
3-chloro-4-pyridinyloxy,
2-(3-pyridinyl)ethoxy,
1-isopropylpiperidin-4-yloxy,
3-azetidinyloxy,
1-methyl-3-azetidinyloxy,
1-isopropyl-3-azetidinyloxy,
1-(2,2,2-trifluoroethyl)piperidin-4-yloxy,
1-methyl-4-pyrazolyl,
3-aminopyridin-4-yl,
1-piperazinylmethyl,
1-piperazinylcarbonyl,
1-methylpiperidin-4-ylaminocarbonyl,
2-(N-morpholinyl)ethoxy,
2-methoxyethoxy,
2-chloropyridin-5-ylmethoxy,
2-chloropyridin-4-ylmethoxy,
1-acetylpiperidin-4-yloxy,
1-(2-fluoroethyl)piperidin-4-yloxy,
1-(2,2-difluorethyl)piperidin-4-yloxy,
1-(2-methoxyethyl)piperidin-4-yloxy,
1-(2-hydroxyethyl)piperidin-4-yloxy,
methylaminocarbonylmethoxy,
aminocarbonylmethoxy,
3-azetidinylmethoxy,
3-(1-methylazetidinyl)methoxy,
2-aminopyridin-4-yl,
6-methoxypyridin-2-ylmethoxy,
5-methylisoxazol-3-yl, and
2-(Pyridin-3-yl)ethoxy;
or R³ can be one of the following heterocyclyloxy groups:

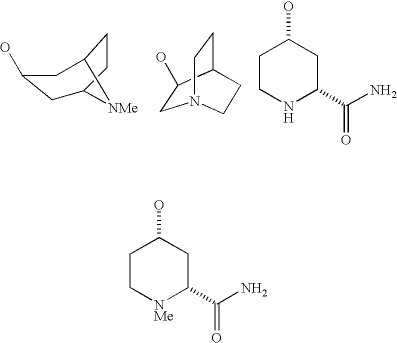

According to some embodiments, Ar is substituted aryl or substituted heteroaryl.

According to some embodiments, Ar is substituted aryl.

According to some embodiments, Ar is aryl, substituted by 1, 2, 3, 4 or 5 substituents independently selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, alkenyl, alkynyl, aryl, cycloalkyl, cycloalkenyl, heterocyclyl, and heteroaryl moieties contained within any of the listed substituted aryl groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio.

According to some embodiments, Ar is aryl substituted by 1, 2, 3, 4 or 5 substituents independently selected from the group consisting of aminosulfonyl, aminocarbonyl, aryl, heteroaryl, heterocyclyl, amino, substituted amino, alkyl, halo, and cyano;

wherein the alkyl, aryl, and heteroaryl moieties contained within any of the listed substituted aryl groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio.

According to some embodiments, Ar is aryl selected from the group consisting of phenyl and naphthyl. Where Ar is phenyl it is often unsubstituted at one or both of the positions adjacent to the NH depicted in Formula I (the ortho positions).

According to some embodiments, Ar is substituted phenyl. In some embodiments, the phenyl is substituted with two or more substituents. In some such embodiments, two adjacent substituents are linked together to form a ring that is fused to the phenyl ring. The fused ring may be saturated, unsaturated or aromatic, and may itself be substituted. Preferred embodiments of these fused ring systems include phenyl fused to a 1,3-dioxolane; phenyl fused to a 1,4-dioxane; phenyl fused to a pyrazole; phenyl fused to imidazole; phenyl fused to triazole; phenyl fused to pyrazole; and phenyl fused to a pyrrolidinyl or piperidinyl ring.

In some embodiments, phenyl is substituted by 1, 2 or 3 groups that are not attached to said phenyl ortho to the NH of formula I.

In certain embodiments, Ar is of the formula:

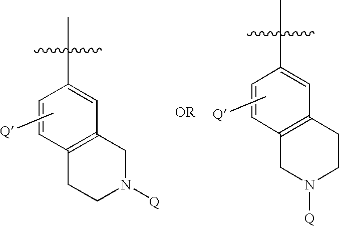

wherein Q is an optionally substituted acyl group;

and Q′ is alkyl, alkoxy, halo, aryl, heteroaryl, aryloxy, heteroaryloxy, heterocyclyloxy, arylalkyl, heteroaryl, or heterocyclyloxy, each of which can be substituted; or Q′ can be H, halo, CN, COOR′, CONR′₂, NR′₂, S(O)_{q}R′, or S(O)_{q}NR′₂, where each R′ is H or C₁-C₄ alkyl.

In many such embodiments, Q′ is H or halo or alkoxy.

Q in these fused systems can be, for example:
1-methylimidazol-2-ylcarbonyl,
3-methoxypropionyl,
1-methylimidazol-5-ylcarbonyl,
N,N-dimethylaminoacetyl,
Tetrahydropyran-4-ylcarbonyl,
1-methylpiperidin-4-ylcarbonyl,
1-methylpiperidin-3-ylcarbonyl,
2-pyridinoyl,
3-pyridinoyl,
4-pyridinoyl,
1-methylpyrrolidin-2-ylcarbonyl,
1-methylpyrrolidin-2S-ylcarbonyl,
(2-N-morpholino)pyridin-5-ylcarbonyl,
(2-N-morpholino)pyridin-4-ylcarbonyl,
(2-N-piperidinyl)pyridin-5-ylcarbonyl,
1-methylpyrazol-4-ylcarbonyl,
1-methylpiperidin-3-ylcarbonyl,
pyridin-3-yl-acetyl,
imidazo[1,2-a]pyridin-4-ylcarbonyl,
2-(piperazine-1-yl)pyridin-5-ylcarbonyl,
2-amino-3-hydroxybutanoyl, or
2S-amino-3R-hydroxybutanoyl.

In many embodiments, Ar is phenyl having either 1 or 2 substituents, or it is a phenyl with an additional ring fused to it. Frequently, Ar is phenyl with a non-hydrogen substituent at one or both of the ‘meta’ positions of the phenyl ring, i.e., it is a 3-substituted phenyl or a 3,5-disubstituted phenyl. In other embodiments, Ar is phenyl with a non-hydrogen substituent at one or both of positions 3 and 4, e.g., it is a 4-substituted phenyl or a 3,4-disubstituted phenyl.

According to some embodiments, Ar is heteroaryl.

According to some embodiments, Ar is heteroaryl selected from the group consisting of pyrrolyl, furanyl, thiophenyl (thienyl), imidazolyl, pyrazolyl, oxazolyl, isoxazolyl, thiazolyl, isothiazolyl, 1,2,3-triazolyl, 1,2,4-triazolyl, oxadiazolyl, thiadiazolyl, tetrazolyl, pyridinyl, pyrazinyl, pyrimidinyl, pyridazinyl, triazinyl, indolyl, isoindole, benzimidazolyl, benzothiophenyl, benzofuranyl, benzoxazolyl, benzothiazolyl, quinolinyl, isoquinolyl, quinazolyl, quinozalyl, cinnolyl, pteridine, carbazole, carboline, phenanthridine, acridine, phenanthroline, phthalazine, naphthylpyridine, phenazine, and purine.

According to some embodiments, Ar is a heteroaryl group selected from the group consisting of 2-pyridinyl, 3-pyridinyl, 4-pyridinyl, 2-pyrimidinyl, 4-pyrimidinyl, 5-pyrimidinyl, 1-pyrazolyl, 3-pyrazolyl, 4-pyrazolyl, 5-pyrazolyl, 2-oxazolyl, 4-oxazolyl, 5-oxazolyl, and 2-thiazolyl, 4-thiazolyl, and 5-thiazolyl.

According to some embodiments, Ar is aryl or heteroaryl, substituted by 1, 2, 3, 4 or 5 substituents independently selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio. Typically in these embodiments, Ar has one or two non-hydrogen substituents.

According to some embodiments, Ar is aryl or heteroaryl, substituted by 1, 2, 3, 4 or 5 substituents independently selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, alkenyl, alkynyl, aryl, cycloalkyl, cycloalkenyl, heterocyclyl, and heteroaryl moieties contained within any of the preceding “substituted aryl” groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio.

According to some embodiments, Ar is heteroaryl, substituted by 1, 2, 3, 4 or 5 substituents independently selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, alkenyl, alkynyl, aryl, cycloalkyl, cycloalkenyl, heterocyclyl, and heteroaryl moieties contained within any of the listed substituted aryl groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio.

According to some embodiments, Ar is aryl or heteroaryl substituted by 1, 2, 3, 4 or 5 substituents independently selected from the group consisting of aminosulfonyl, aminocarbonyl, aryl, heteroaryl, heteroaryl, heterocyclyl, amino, substituted amino, alkyl, alkyl, halo, and cyano;

wherein the alkyl, aryl, heterocyclyl, and heteroaryl moieties contained within any of the preceding “substituted aryl” groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio.

According to some embodiments, Ar is heteroaryl substituted by 1, 2, 3, 4 or 5 substituents independently selected from the group consisting of aminosulfonyl, aminocarbonyl, aryl, heteroaryl, heterocyclyl, amino, substituted amino, alkyl, halo, and cyano;

wherein the alkyl, aryl, and heteroaryl moieties contained within any of the listed substituted aryl groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio.

According to some embodiments, Ar is a 5- or 6-membered heteroaryl group having 1 or 2 heteroatoms as ring members, independently selected from the group consisting of O, S and N, that is optionally substituted with 1, 2 or 3 substituents selected from the group consisting of aminosulfonyl, aminocarbonyl, aryl, heteroaryl, heterocyclyl, amino, substituted amino, alkyl, halo, and cyano.

According to some embodiments, Ar is selected from the group consisting of substituted pyridyl, substituted pyrazolyl, substituted thiazolyl, substituted pyrimidyl, substituted pyridazinyl, substituted oxazolyl and substituted isoxazolyl.

Specific examples of preferred Ar groups include the following:
Phenyl,
3-aminocarbonyl-5-N-(morpholino)phenyl,
4-aminocarbonylphenyl,
3-methanesulfonamidophenyl,
3-acetylamino-5-N-morpholinomethyl-phenyl,
3-methoxy-5-(5-methyl-1-tetrazolyl)-phenyl,
4-(N-morpholino)phenyl,
4-(N-morpholinocarbonyl)phenyl,
3-(5-oxazolyl)-phenyl,
3-(1-hydroxy-1-methyl)-1-ethylphenyl,
3-(1-hydroxyethyl)phenyl,
3-fluorophenyl,
2-fluorophenyl,
3,4,5-trifluorophenyl,
3-trifluoromethylphenyl,
2-trifluoromethylpyridin-5-yl,
3-(1-methylpyrazol-3-yl)phenyl,
3-(2-amino-4-pyridyl)-5-acetylaminophenyl,
3-pyrrolidinylcarbonylamino-5-(N-morpholino)methyl-phenyl,
3-acetylamino-5-(2-methoxy-5-pyridinyl)phenyl,
3-acetylamino-5-(4-pyrazolyl)phenyl,
3-methoxycarbonylamino-5-(pyrazol-4-yl)phenyl,
3-aminocarbonyl-5-(4-pyrazolyl)phenyl,
3-aminocarbonyl-5-(1-methyl-4-pyrazolyl)phenyl,
3,5-bis(aminocarbonyl)phenyl,
3,5-bis(acetylamino)phenyl,
3-aminocarbonyl-5-(4-methyl-1-piperazinyl)phenyl,
4-(3-pyrazolyl)phenyl,
3-aminocarbonyl-5-(2-methoxy-5-pyridinyl)phenyl,
3-aminocarbonyl-5-(6-methoxy-2-pyrazinyl)phenyl,
3-aminocarbonyl-5-(N-methylpiperidin-2-on-5-yl)phenyl,
3-(5-methyl-1-tetrazolyl)phenyl,
3-aminocarbonyl-5-(2-pyrazinyl)phenyl,
3-(methoxycarbonylamino)-5-(2-(N-morpholino)pyrimidin-5-yl)phenyl,
3-(N-morpholinocarbonyl)-5-(N-morpholino)phenyl,
3-aminocarbonyl-5-(1-methyl-4-pyrazolyl)phenyl,
3-aminocarbonyl-5-(5-pyrimidinyl)phenyl,
3-(N-methylaminocarbonyl)phenyl,
4-(aminocarbonyl)phenyl,
4-(N-methylaminocarbonyl)phenyl,
3-(1,2,4-triazolyl-1-methyl)phenyl,
3-(1,2,4-triazolyl-4-methyl)phenyl,
3-(N-morpholinocarbonylmethyl)phenyl,
3-methoxyphenyl,
3-methoxy-4-fluorophenyl,
3-(1-methyl-3-pyrazolyl)phenyl,
(3-N-morpholinomethyl)phenyl,
(2-trifluoromethyl)benzimidazol-6-yl,
benzopyrazol-6-yl,
3-fluoro-4-isopropylphenyl,
3-(2-oxopyrrolidin-1-yl)phenyl,
3-methoxy-4-(5-oxazolyl)phenyl,
3-methanesulfonylphenyl,
3-methoxy-4-(isobutyrylamino)phenyl,
3-(1-pyrazolyl)phenyl,
4-(N-pyrrolidinylcarbonyl)phenyl,
benzimidazol-5-yl,
3-(4-methyl-1,2,4-triazol-3-yl)phenyl,
4-pyridyl,
3-pyridyl,
3-(N-morpholinylcarbonylmethyl)phenyl,
3-cyano-5-fluorophenyl,
4-(methylaminocarbonylmethyl)phenyl,
3-(methylaminocarbonylmethyl)phenyl,
3-(isopropylaminocarbonylmethyl)phenyl,
3-(cyclopropylaminocarbonylmethyl)phenyl,
4-(isopropylaminocarbonylmethyl)phenyl,
4-(cyclopropylaminocarbonylmethyl)phenyl,
3-(imidazol-2-yl)phenyl,
3-(2-methyl-thiazol-4-yl)phenyl,
3-(5-pyrimidinyl)phenyl,
3-(2-methoxypyrimidin-5-yl)phenyl,
3-(1,3,5-trimethylpyrazol-4-yl)phenyl,
3-(4,5-dimethyloxazol-2-yl)phenyl,
3-(5-methylfuran-2-yl)phenyl,
3-(methylaminocarbonyl-5-(1-methylpyrazol-4-yl)phenyl,
3-(methoxycarbonylamino)-5-(2-N-morpholinopyrimidin-5-yl)phenyl,
3-(4-morpholino)-5-(4-morpholinocarbonyl)phenyl,
3-(methoxycarbonylamino)-5-(pyrazol-4-yl)phenyl,
3-(methoxycarbonylamino)-5-(morpholin-4-ylmethyl)phenyl,
3-(aminocarbonyl)-5-(4-morpholinyl)phenyl,
3-methoxy-5-trifluoromethylphenyl,
3-(aminocarbonylmethoxy)-5-(1-methylpyrazol-4-yl)phenyl,
3,5-dimethoxyphenyl,
3,5-difluorophenyl,
3,4-difluorophenyl,
3,4,5-trifluorophenyl,
3-chloro-4-fluorophenyl,
3-fluoro-4-(thiazol-2-yl)phenyl,
3-ethoxyphenyl,
4-(oxazol-5-yl)phenyl,
3-fluoro-4-(morpholin-4-yl)phenyl,
4-(isopropylaminocarbonyl)phenyl,
4-(morpholin-4-ylsulfonyl)phenyl,
4-cyclohexylphenyl,
4-(pyrimidin-5-yl)phenyl,
4-(isoxazol-4-yl)phenyl,
2-fluorophenyl,
2,3-difluorophenyl,
2,4-difluorophenyl,
benzotriazol-5-yl,
4-aminosulfonylphenyl,
4-isopropylphenyl,
3-fluoro-4-isopropylphenyl,
3-methoxy-4-(oxazol-5-yl)phenyl,
4-(methylaminosulfonylmethyl)phenyl,
3-(aminosulfonyl)phenyl,
4-(1-methyl-4-pyrazolyl)phenyl,
2-methoxyphenyl,
2-trifluoropyridin-5-yl,
4-(isopropylaminosulfonyl)phenyl,
4-cyanophenyl,
4-ethoxyphenyl,
3-difluormethoxyphenyl,
3,4-dioxolanylphenyl,
3,4-dimethoxyphenyl,
3,4-dioxanylphenyl,
3-propoxyphenyl,
3-hydroxyphenyl,
4-(1-pyrazolylmethyl)phenyl,
4-(1,2,4-triazol-1-ylmethyl)phenyl,
4-(1-pyrazol-1-ylmethyl)phenyl,
4-(2-methylthiazol-4-yl)phenyl,
4-(1,2,3-thiadiazol-4-yl)phenyl,
4-(oxazolin-2-on-4-yl)phenyl,
4-(2-oxazolyl)phenyl,
3-[2-(2-hydroxypyridin-5-ylcarbonylamino)-ethoxy]-5-(1-methylpyrazol-4-yl)phenyl,
3-fluoro-4-(imidazol-2-yl)phenyl,
4-(3-methoxypyridin-5-yl)phenyl,
3-(2-methoxypyridin-5-yl)phenyl,
3-(3-pyridinyl)phenyl,
3-(dimethylaminomethyl)-5-(methanesulfonamido)phenyl,
3-(4-methylpiperazin-ylethyl)-5-(methanesulfonamido)phenyl,
1-methylbenzimidazol-2-yl,
5,6-dimethylbenzimidazol-2-yl,
4,5-dicyanoimidazol-2-yl,
3-amino-5-aminocarbonylmethylphenyl,
imidazol-1-yl,
2-(pyridin-2-yl)ethyl,
imidazol-1-ylmethylphenyl,
3-(acetylamino)-5-(N-methyl-2-pyridon-5-yl)phenyl,
3-(acetylamino)-5-iodophenyl,
3-(acetylamino)-5-(pyridin-3-yl)phenyl,
4-(N-morpholinylsulfonyl)phenyl,
3-(acetylamino)-5-(dimethylaminomethyl)phenyl,
4-(tetrazol-5-yl)phenyl,
3-(tetrazol-5-yl)phenyl,
3-chloro-4-(N-morpholinocarbonyl)phenyl,
3-(tetrazol-1-yl)phenyl,
3-aminocarbonyl-4-(N-morpholino)phenyl,
3-acetylamino-5-(pyrrolidin-1-ylmethyl)phenyl,
6-chlorobenzopyrazol-4-yl,
6-fluorobenzopyrazol-4-yl,
3-acetylamino-5-aminomethylphenyl,
3-(acetylamino)-5-(piperazin-1-ylmethyl)phenyl,
3-(isobutyrylamino)-5-(N-morpholinomethyl)phenyl,
3-(methylsulfonamido)-5-(N-morpholinomethyl)phenyl,
3-(N-morpholinomethyl)-5-(5-tetrazolyl)phenyl,
3-acetylamino-5-(pyridin-4-yl)phenyl,
3-cyano-5-(N-morpholinomethyl)phenyl,
3-methoxy-5-(5-methyltetrazol-1-yl)phenyl,
3-methoxy-5-(tetrazol-1-yl)phenyl,
3-(4-morpholino)-5-(pyrazol-4-yl)phenyl,
3-(4-morpholino)-5-(pyridine-4-yl)phenyl,
3-(4-morpholino)-5-(3-fluoropyridine-4-yl)phenyl,
3-(4-morpholino)-5-(pyridine-3-yl)phenyl,
3-aminocarbonyl-5-(N-morpholinomethyl)phenyl,
3-(methoxycarbonylamino)-5-(morpholin-4-ylmethyl)phenyl,
3-(4-trifluoromethylpyrazol-2-yl)phenyl,
3-(cyclopropylaminocarbonyl)-5-(1-methylpyrazol-4-yl)phenyl,
3-(1-methylpyrazol-4-yl)-5-((1-methylpiperidin-4-yl)aminocarbonyl)phenyl,
3-(methylaminocarbonyl)-5-(1-methylpyrazol-4-yl)phenyl,
3-(2-methoxyethylaminocarbonyl)-5-(1-methylpyrazol-4-yl)phenyl,
3-(aminocarbonyl)-5-fluorophenyl,
3-methylaminocarbonyl-5-(pyrimidin-5-yl)phenyl,
3-methylaminocarbonyl-5-(pyridine-4-yl)phenyl,
3-methylaminocarbonyl-5-(pyridine-3-yl)phenyl,
3-amino-5-(N-morpholinomethyl)phenyl,
3-(cyanomethyl)-5-Methylpyrazol-4-yl)phenyl,
3-(aminocarbonyl)-5-(aminocarbonylmethoxy)phenyl,
4-(isobutyrylamino)-3-methoxyphenyl,
3-(isobutyrylamino)-5-methoxyphenyl,
3-(aminocarbonylmethyl)-5-(N-morpholinomethyl)phenyl,
3-cyano-4-(1,2,4-triazol-1-ylmethyl)phenyl,
3-(methoxycarbonylmethoxy)-5-(1-methylpyrazol-4-yl)phenyl,
3-(hydroxycarbonylmethoxy)-5-(1-methylpyrazol-4-yl)phenyl,
3-(cyclopropylaminocarbonylmethoxy)-5-(1-methylpyrazol-4-yl)phenyl,
3-(2-(pyrrolidin-1-yl)ethylaminocarbonylmethoxy)-5-(1-methylpyrazol-4-yl)phenyl,
3-(2-(methoxyethoxy)carbonylmethoxy)-5-(1-methyl pyrazol-4-yl)phenyl,
3-(2-(2-oxopyrrolidin-1-yl)ethylaminocarbonylmethoxy)-5-(1-methylpyrazol-4-yl)phenyl,
3-((2-tetrahydrofuranyl)methylaminocarbonylmethoxy)-5-(1-methylpyrazol-4-yl)phenyl,
3-((2-tetrahydrofuranyl)methylaminocarbonylmethoxy)-5-(1-methylpyrazol-4-yl)phenyl,
3-((2-S-tetrahydrofuranyl)methylaminocarbonylmethoxy)-5-(1-methylpyrazol-4-yl)phenyl,
3-((2-R-tetrahydrofuranyl)methylaminocarbonylmethoxy)-5-(1-methylpyrazol-4-yl)phenyl,
3-((1-methylpiperidin-4-yl)aminocarbonylmethoxy)-5-(1-methylpyrazol-4-yl)phenyl,
3-((2-methylsulfonylethyl)aminocarbonylmethoxy)-5-(1-methylpyrazol-4-yl)phenyl,
3-((2-acetylaminoethyl)aminocarbonylmethoxy)-5-(1-methylpyrazol-4-yl)phenyl,
3-(tetrahydropyran-4-ylaminocarbonylmethoxy)-5-(1-methylpyrazol-4-yl)phenyl,
3-((2-hydroxypropyl)aminocarbonylmethoxy)-5-(1-methylpyrazol-4-yl)phenyl,
3-(pyridine-2-ylmethylaminocarbonylmethoxy)-5-(1-methylpyrazol-4-yl)phenyl,
3-(cyclohexylmethylaminocarbonylmethoxy)-5-(1-methylpyrazol-4-yl)phenyl,
3-(pyridine-3-ylmethylaminocarbonylmethoxy)-5-(1-methylpyrazol-4-yl)phenyl,
3-(N,N-dimethylaminocarbonylmethoxy)-5-(1-methylpyrazol-4-yl)phenyl,
3-(N-morpholinocarbonylmethoxy)-5-(1-methylpyrazol-4-yl)phenyl,
3-(4-methylpiperazin-1-ylcarbonylmethoxy)-5-(1-methylpyrazol-4-yl)phenyl,
3-(3-oxopiperazin-1-ylcarbonylmethoxy)-5-(1-methylpyrazol-4-yl)phenyl,
3-(4-dimethylaminopiperidin-1-ylcarbonylmethoxy)-5-(1-methylpyrazol-4-yl)phenyl,
4-(methylaminosulfonylmethyl)phenyl,
3-(cyclopropylaminocarbonyl)-5-(N-morpholinyl)phenyl,
3-(methylaminocarbonyl)-5-(N-morpholinyl)phenyl,
3-(3-oxomorpholin-4-ylmethyl)phenyl,
3-(2-oxopyrrolidin-1-ylmethyl)phenyl,
3-(2-oxooxazol-1-ylmethyl)phenyl,
3-(methylaminocarbonylmethoxy)-5-(1-methylpyrazol-4-yl)phenyl,
4-(N-morpholinomethyl)phenyl,
3-(3-methoxypropanoylaminomethyl)phenyl,
3-(isoxazol-5-ylcarbonylaminomethyl)phenyl,
3-(tetrahydrofuran-2R-ylcarbonylaminomethyl)phenyl,
3-(tetrahydrofuran-2S-ylcarbonylaminomethyl)phenyl,
3-(1-acetylpyrrolidin-2R-ylcarbonylaminomethyl)phenyl,
3-(1-acetylpyrrolidin-2S-ylcarbonylaminomethyl)phenyl,
3-(pyridin-3-ylmethylacetylaminomethyl)phenyl,
3-(pyridin-3-ylaminomethyl)phenyl,
3-(cyclopropylsulfonylaminomethyl)phenyl,
3-(ethoxycarbonylaminomethyl)phenyl,
3-hydroxy-5-(methylaminocarbonyl)phenyl,
3-ethoxy-5-(methylaminocarbonyl)phenyl,
3-(methylaminocarbonylmethyl)-5-(N-morpholinomethyl)phenyl,
3-(N,N-diethylaminocarbonylmethyl)-5-(N-morpholinomethyl)phenyl,
3-(isopropylaminocarbonylmethyl)-5-(N-morpholinomethyl)phenyl,
3-(tetrahydropyran-4-ylaminocarbonylmethyl)-5-(N-morpholinomethyl)phenyl,
3-(t-butoxycarbonylaminomethyl)phenyl,
3-(methylaminocarbonyl)-5-(5-(4-methylpiperazin-1-yl))phenyl,
2-methoxyphenyl,
2-trifluoropyridin-5-yl,
3-(2-(pyrrolidin-1-yl)ethylaminosulfonyl)phenyl,
3-(cyclopropylaminocarbonylmethyl)phenyl,
3-(2R-(1-methylpyrrolidin-2-ylcarbonylamino)propoxy)-5-(1-methylpyrazol-4-yl)phenyl,
3-(2S-(1-methylpyrrolidin-2-ylcarbonylamino)propoxy)-5-(1-methylpyrazol-4-yl)phenyl,
3-(1-methoxycarbonylpiperidin-4-yl)phenyl,
3-(methoxycarbonylamino)-5-(2-(morpholin-4-yl)pyrimidin-5-yl)phenyl,
3-(2-(1-pyrrolidinyl)ethylaminocarbonylmethoxy)-5-(1-methylpyrazol-4-yl)phenyl,
3-n-propoxyphenyl,
3-(N-(1-methylpiperidin-4-ylcarbonyl)piperidin-4-yl)phenyl,
3-(N—(N,N-dimethylaminoacetyl)piperidin-4-yl)phenyl,
4-(ethoxycarbonylaminomethyl)phenyl,
4-(methylsulfonylmethyl)phenyl,
4-(1-methylimidazol-5-ylcarbonylaminomethyl)phenyl,
4-(1-methylpiperidin-4-ylcarbonylaminomethyl)phenyl,
3,5-dimethoxyphenyl,
3-(1-methylpiperidin-4-ylcarbonylaminomethyl)phenyl,
4-fluoro-3-(N-morpholinocarbonylmethyl)phenyl,
3-(2-(N-morpholino)ethyloxy)-5-(1-methylpyrazol-4-yl)phenyl,
3-(trans-(2-dimethylamino)cyclohexylaminocarbonylmethoxy)-5-(1-methylpyrazol-4-yl)phenyl,
3-(3-(dimethylamino)propoxy)-5-(1-methylpyrazol-4-yl)phenyl,
3-(2-(pyrrolidin-1-yl)ethoxy)-5-(1-methylpyrazol-4-yl)phenyl,
3-(2-(N-morpholino)ethoxy)-5-(1-methylpyrazol-4-yl)phenyl,
3-(2-(N-piperidinyl)ethoxy)-5-(1-methylpyrazol-4-yl)phenyl,
3-(2-(ethoxycarbonylamino)ethoxy)-5-(1-methylpyrazol-4-yl)phenyl,
3-(2-(pyrrolidin-2-on-1-ylacetylamino)ethoxy)-5-(1-methylpyrazol-4-yl)phenyl,
3-(2-(isoxazol-2-ylcarbonylamino)ethoxy)-5-(1-methylpyrazol-4-yl)phenyl,
3-(2-(N-pyrrolidinylacetylcarbonylamino)ethoxy)-5-(1-methylpyrazol-4-yl)phenyl,
3-(2-(tetrahydrofuran-2R-ylcarbonylamino)ethoxy)-5-(1-methylpyrazol-4-yl)phenyl,
3-(2-(tetrahydrofuran-2S-ylcarbonylamino)ethoxy)-5-(1-methylpyrazol-4-yl)phenyl,
3-(2-(1-methylpyrazol-3-ylcarbonylamino)ethoxy)-5-(1-methylpyrazol-4-yl)phenyl,
3-(2-(4-chloropyridin-2-ylcarbonylamino)ethoxy)-5-(1-methylpyrazol-4-yl)phenyl,
3-(2-(piperidin-3R-ylcarbonylamino)ethoxy)-5-(1-methylpyrazol-4-yl)phenyl,
3-(2-(piperidin-3S-ylcarbonylamino)ethoxy)-5-(1-methylpyrazol-4-yl)phenyl,
3-(2-(piperidin-4-ylmethylcarbonylamino)ethoxy)-5-(1-methylpyrazol-4-yl)phenyl,
3-(2-(pyrrolidin-2R-ylcarbonylamino)ethoxy)-5-(1-methylpyrazol-4-yl)phenyl,
3-(2-(pyrrolidin-2S-ylcarbonylamino)ethoxy)-5-(1-methylpyrazol-4-yl)phenyl,
3-(2-(2R-amino-3 S-hydroxybutanoylamino)ethoxy)-5-(1-methylpyrazol-4-yl)phenyl,
3-(2-(2S-amino-3R-hydroxybutanoylamino)ethoxy)-5-(1-methylpyrazol-4-yl)phenyl,
3-(2-(1-methylpiperidin-3R-ylcarbonylamino)ethoxy)-5-(1-methylpyrazol-4-yl)phenyl,
3-(2-(1-methylpiperidin-3S-ylcarbonylamino)ethoxy)-5-(1-methylpyrazol-4-yl)phenyl,
3-(2-(1-methylpiperidin-4-ylmethylcarbonylamino)ethoxy)-5-(1-methylpyrazol-4-yl)phenyl,
3-(2-(1-methylpyrrolidin-2R-ylcarbonylamino)ethoxy)-5-(1-methylpyrazol-4-yl)phenyl,
3-(2-(piperidin-4-ylmethylcarbonylamino)ethoxy)-5-(1-methylpyrazol-4-yl)phenyl,
3-(4-dimethylaminopiperidin-1-ylcarbonylmethyl)-5-(1-methylpyrazol-4-yl)phenyl,
3-(pyrrolidin-2R-ylmethoxy)-5-(1-methylpyrazol-4-yl)phenyl,
3-(pyrrolidin-2-ylmethoxy)-5-(1-methylpyrazol-4-yl)phenyl,
3-(pyrrolidin-2S-ylmethoxy)-5-(1-methylpyrazol-4-yl)phenyl,
3-(3-hydroxy-2-aminopropoxy)-5-(1-methylpyrazol-4-yl)phenyl,
3-(2R—(N,N-dimethylaminoacetylamino)propoxy)-5-(1-methylpyrazol-4-yl)phenyl,
3-(2S—(N,N-dimethylaminoacetylamino)propoxy)-5-(1-methylpyrazol-4-yl)phenyl,
3-(2R—(N,N-dimethylaminopropoxy)-5-(1-methylpyrazol-4-yl)phenyl,
3-(2S—(N,N-dimethylaminopropoxy)-5-(1-methyl pyrazol-4-yl)phenyl,
3-(2-(4-methylpiperidin-1-ylacetylamino)ethoxy)-5-(1-methylpyrazol-4-yl)phenyl,
3-(2-aminoethylaminocarbonyl)-5-(1-methylpyrazol-4-yl)phenyl,
3-(2-(N,N-dimethylamino)ethylaminocarbonyl)-5-(1-methylpyrazol-4-yl)phenyl,
3-(2-(N,N-diethylamino)ethylaminocarbonyl)-5-(1-methylpyrazol-4-yl)phenyl,
3-(2-(acetylamino)ethylaminocarbonyl)-5-(1-methylpyrazol-4-yl)phenyl,
3-((pyrrolidin-5-on-2-yl)methylaminocarbonyl))-5-(1-methylpyrazol-4-yl)phenyl,
3-(2-(3R-hydroxypyrrolidin-1-yl-acetylamino)ethoxy)-5-(1-methylpyrazol-4-yl)phenyl,
3-(2-(3S-hydroxypyrrolidin-1-yl-acetylamino)ethoxy)-5-(1-methylpyrazol-4-yl)phenyl,
3-(2-(1,2S-dimethylpiperidin-3S-yl-carbonylamino)ethoxy)-5-(1-methylpyrazol-4-yl)phenyl,
3-(2-(2S—N,N-dimethylaminopropionyl)aminoethoxy)-5-(1-methylpyrazol-4-yl)phenyl,
3-(2R-(2-pyridinylamino)propoxy)-5-(1-methylpyrazol-4-yl)phenyl,
3-(2S-(pyrrolidin-1-ylacetylamino)propoxy)-5-(1-methylpyrazol-4-yl)phenyl,
3-(2R-(pyrrolidin-1-ylacetylamino)propoxy)-5-(1-methylpyrazol-4-yl)phenyl,
3-(2R-(1-methylimidazol-4-carbonylamino)propoxy)-5-(1-methylpyrazol-4-yl)phenyl,
3-(2R-(1-methylpiperidinyl-3-carbonylamino)propoxy)-5-(1-methylpyrazol-4-yl)phenyl,
3-(2R-(tetrahydrofuran-2R-ylcarbonylamino)propoxy)-5-(1-methylpyrazol-4-yl)phenyl,
3-(2R-(tetrahydrofuran-2S-ylcarbonylamino)propoxy)-5-(1-methylpyrazol-4-yl)phenyl,
3-(2R-(3-methoxypropionylamino)propyl)-5-(1-methylpyrazol-4-yl)phenyl,
3-(3-hydroxypropyl)-5-(1-methylpyrazol-4-yl)phenyl
3-(2-(1-t-butoxycarbonyl-2S-methylpiperidin-3S-yl-carbonylamino)ethoxy)-5-(1-methylpyrazol-4-yl)phenyl,
3-(2-(1-methyl-3S-methylpiperidin-4R-yl-carbonylamino)ethoxy)-5-(1-methylpyrazol-4-yl)phenyl,
3-(2-(3R-fluoropyrrolidin-1-ylacetylamino)ethoxy)-5-(1-methylpyrazol-4-yl)phenyl,
3-(2-(3R-dimethylaminopyrrolidin-1-ylacetylamino)ethoxy)-5-(1-methylpyrazol-4-yl)phenyl,
3-(2-(3S-fluoropyrrodin-1-ylacetylamino)ethoxy)-5-(1-methylpyrazol-4-yl)phenyl,
3-(2-methylthiazol-4-yl)phenyl,
3-(2-(pyridin-2-ylmethylaminoacetylamino)ethoxy)-5-(1-methylpyrazol-4-yl)phenyl,
3-(2-(2-aminocarbonyl)pyrrolidin-1-ylacetyl amino)ethoxy)-5-(1-methylpyrazol-4-yl)phenyl,
3-(2-(4S-fluoropyrrolidin-2S-ylcarbonylamino)ethoxy)-5-(1-methylpyrazol-4-yl)phenyl,
3-(2-(4S-hydroxypyrrolidin-2S-ylcarbonylamino)ethoxy)-5-(1-methylpyrazol-4-yl)phenyl,
3-(2-(1-methyl-4S-fluoropyrrolidin-2S-ylcarbonylamino)ethoxy)-5-(1-methylpyrazol-4-yl)phenyl,
3-(2-(1-methyl-4S-hydroxypyrrolidin-2S-ylcarbonylamino)ethoxy)-5-(1-methylpyrazol-4-yl)phenyl,
3-(2-(4,4-difluoropyrrolidin-2S-ylcarbonylamino)ethoxy)-5-(1-methylpyrazol-4-yl)phenyl,
3-(2-(4,4-difluoropyrrolidin-2R-ylcarbonylamino)ethoxy)-5-(1-methylpyrazol-4-yl)phenyl,
3-(2-(1-methyl-4,4-difluoropyrrolidin-2S-ylcarbonylamino)ethoxy)-5-(1-methylpyrazol-4-yl)phenyl,
3-(2-(1-methyl-4,4-difluoropyrrolidin-2R-ylcarbonylamino)ethoxy)-5-(1-methylpyrazol-4-yl)phenyl,
3-(2-(4S-hydroxypyrrolidin-2S-ylcarbonylamino)ethoxy)-5-(1-methylpyrazol-4-yl)phenyl,
3-(2-(4-fluoropyrrolidin-2-ylcarbonylamino)ethoxy)-5-(1-methylpyrazol-4-yl)phenyl,
3-(2-(4-hydroxypyrrolidin-2-ylcarbonylamino)ethoxy)-5-(1-methylpyrazol-4-yl)phenyl,
3-(2R-(2S-methylpiperidin-3S-yl-carbonylamino)propoxy)-5-(1-methylpyrazol-4-yl)phenyl,
3-(2-aminoethoxy)-5-(thiazol-5-yl)phenyl,
3-(2-aminoethoxy)-5-(1-methylimidazol-5-yl)phenyl,
3-(2-methoxyethoxy)-5-(1-methylpyrazol-4-yl)phenyl,
3-(2-(2-aminopyridin-4-ylcarbonylamino)ethoxy)-5-(1-methylpyrazol-4-yl)phenyl,
3-(2-(2-hydroxypyridin-4-ylcarbonylamino)ethoxy)-5-(1-methylpyrazol-4-yl)phenyl,
3-(2-pyridylmethoxy)-5-(1-methylpyrazol-4-yl)phenyl,
3-(2-aminopyridin-4-ylmethoxy)-5-(1-methylpyrazol-4-yl)phenyl,
3-((2-(4-methylpiperazin-1-yl)ethylaminocarbonyl)methoxy)-5-(1-methylpyrazol-4-yl)phenyl,
3-(2-(dimethylamino)ethoxy)-5-(pyrimidin-5-yl)phenyl,
3-(2-(3-methoxypropionylamino)ethoxy)-5-(pyrimidin-5-yl)phenyl,
3-(2-(1-methylpyrrolidin-2S-ylcarbonylamino)ethoxy)-5-(pyrimidin-5-yl)phenyl,
3-(1-methylimidazol-5-yl)-5-(N-morpholinomethyl)phenyl,
3-(1-aminocycloprop-1-ylmethoxy)-5-(1-methylpyrazol-4-yl)phenyl,
3-(3-azetidinylmethoxy)-5-(1-methylpyrazol-4-yl)phenyl,
3-(2S-amino-3-hydroxypropyl)-5-(1-methylpyrazol-4-yl)phenyl,
3-(2R-(methylaminocarbonyl)pyrrolidin-4R-yloxy)-5-(1-methylpyrazol-4-yl)phenyl,
3-((imidazo[1,2-a]pyridin-4-ylcarbonyl)aminomethyl)phenyl,
3-(N-acetylpyrrolidin-2-ylcarbonylaminomethyl)phenyl,
3-(2-morpholinopyridin-5-yl)carbonylaminomethylphenyl,
3-(2-(cyclopropylsulfonylamino)ethoxy)phenyl,
3-(2-(N,N-diethylaminoethyl)-N-methylcarbonylmethoxy)-5-(1-methylpyrazol-4-yl)phenyl,
3-((4-N,N-dimethylaminocyclohexylamino)carbonylmethoxy)-5-(1-methylpyrazol-4-yl)phenyl,
3-(4-(2-methoxyethoxy)piperazin-1-ylaminocarbonylmethoxy)-5-(1-methylpyrazol-4-yl)phenyl,
3-(2-(1-methylpyrrolidin-2-yl)ethylaminocarbonylmethoxy)-5-(1-methylpyrazol-4-yl)phenyl,
3-(3S-quinuclidylaminocarbonylmethoxy)-5-(1-methylpyrazol-4-yl)phenyl,
3-(3R-quinuclidylaminocarbonylmethoxy)-5-(1-methylpyrazol-4-yl)phenyl,
3-(N-ethylpyrrolidin-2S-ylmethylaminocarbonylmethoxy)-5-(1-methylpyrazol-4-yl)phenyl,
3-(N-ethylpyrrolidin-2R-ylmethylaminocarbonylmethoxy)-5-(1-methylpyrazol-4-yl)phenyl,
3-(4-methyl-1,4-diazepin-1-ylcarbonylmethoxy)-5-(1-methylpyrazol-4-yl)phenyl,
3-(pyrrolidino[carbonylmethoxy)-5-(1-methylpyrazol-4-yl)phenyl,
3-(N-tetrahydropyran-4-yl-N-methylaminocarbonylmethoxy)-5-(1-methylpyrazol-4-yl)phenyl,
3-(N-tetrahydropyran-3-ylaminocarbonylmethoxy)-5-(1-methylpyrazol-4-yl)phenyl,
3-(N-tetrahydropyran-4-yl)methylaminocarbonylmethoxy)-5-(1-methylpyrazol-4-yl)phenyl,
3-(2-(1,2,4,-triazol-1-yl)ethylaminocarbonylmethoxy)-5-(1-methylpyrazol-4-yl)phenyl,
3-((pyrrolidin-2-on-5-ylmethyl)aminocarbonylmethoxy)-5-(1-methylpyrazol-4-yl)phenyl,
3-((pyridin-2-on-4-ylmethyl)aminocarbonylmethoxy)-5-(1-methylpyrazol-4-yl)phenyl,
3-((2R-aminocyclohex-1R-yl)aminocarbonylmethoxy)-5-(1-methylpyrazol-4-yl)phenyl,
3-(2-(4-methylpiperazin-1-yl)ethylaminocarbonylmethoxy)-5-(1-methylpyrazol-4-yl)phenyl,
3-((2R—N,N-dimethylaminocyclohex-1R-yl)aminocarbonylmethoxy)-5-(1-methylpyrazol-4-yl)phenyl,
3-((2S—N,N-dimethylaminocyclohex-1 S-yl)aminocarbonylmethoxy)-5-(1-methylpyrazol-4-yl)phenyl,
3-(2-(pyrrolidin-2-on-1-ylmethyl)carbonylamino)ethoxy)-5-(1-methylpyrazol-4-yl)phenyl,
3-(2-(ethoxycarbonylamino)ethoxy)-5-(1-methylpyrazol-4-yl)phenyl,
3-(2-(N-morpholinocarbonylamino)ethoxy)-5-(1-methylpyrazol-4-yl)phenyl,
3-(2-(1-methylpyrazol-4-ylcarbonylamino)ethoxy)-5-(1-methylpyrazol-4-yl)phenyl,
3-(2-(isoxazol-5-ylcarbonylamino)ethoxy)-5-(1-methylpyrazol-4-yl)phenyl,
3-(2-(pyrrolidin-2R-ylcarbonylamino)ethoxy)-5-(1-methylpyrazol-4-yl)phenyl,
3-(2-(pyrrolidin-2S-ylcarbonylamino)ethoxy)-5-(1-methylpyrazol-4-yl)phenyl,
3-(2-(piperidin-2R-ylcarbonylamino)ethoxy)-5-(1-methylpyrazol-4-yl)phenyl,
3-(2-(piperidin-2S-ylcarbonylamino)ethoxy)-5-(1-methylpyrazol-4-yl)phenyl,
3-(2-(piperidin-3R-ylcarbonylamino)ethoxy)-5-(1-methylpyrazol-4-yl)phenyl,
3-(2-(piperidin-3 S-ylcarbonylamino)ethoxy)-5-(1-methylpyrazol-4-yl)phenyl,
3-(2-(1-methylpyrrolidin-2R-ylcarbonylamino)ethoxy)-5-(1-methylpyrazol-4-yl)phenyl,
3-(2-(1-methylpyrrolidin-2S-ylcarbonylamino)ethoxy)-5-(1-methylpyrazol-4-yl)phenyl,
3-(2-(1-methylpiperidin-2R-ylcarbonylamino)ethoxy)-5-(1-methylpyrazol-4-yl)phenyl,
3-(2-(1-methylpiperidin-2S-ylcarbonylamino)ethoxy)-5-(1-methylpyrazol-4-yl)phenyl,
3-(2-(1-methylpiperidin-3R-ylcarbonylamino)ethoxy)-5-(1-methylpyrazol-4-yl)phenyl,
3-(2-(1-methylpiperidin-3 S-ylcarbonylamino)ethoxy)-5-(1-methylpyrazol-4-yl)phenyl,
3-(2-(2R-aminopropionylamino)ethoxy)-5-(1-methylpyrazol-4-yl)phenyl,
3-(2-(2 S-aminopropionylamino)ethoxy)-5-(1-methylpyrazol-4-yl)phenyl,
3-(2-(2R-methylpiperidin-3 S-ylcarbonylamino)ethoxy)-5-(1-methylpyrazol-4-yl)phenyl,
3-(2-(2 S-methylpiperidin-3R-ylcarbonylamino)ethoxy)-5-(1-methylpyrazol-4-yl)phenyl,
3-(2-(4-methylpiperidin-1-ylaminomethylcarbonyl)ethoxy)-5-(1-methylpyrazol-4-yl)phenyl,
3-(2-(2R—N,N-dimethylaminopropionylamino)ethoxy)-5-(1-methylpyrazol-4-yl)phenyl,
3-(2-(2 S—N,N-dimethylaminopropionylamino)ethoxy)-5-(1-methylpyrazol-4-yl)phenyl,
3-(2-(3R-hydroxypyrrolidin-1-ylmethylcarbonylamino)ethoxy)-5-(1-methylpyrazol-4-yl)phenyl,
3-(2-(3S-hydroxypyrrolidin-1-ylmethylcarbonylamino)ethoxy)-5-(1-methylpyrazol-4-yl)phenyl,
3-(2-(3R-fluoropyrrolidin-1-ylmethylcarbonylamino)ethoxy)-5-(1-methylpyrazol-4-yl)phenyl,
3-(2-(3S-fluoropyrrolidin-1-ylmethylcarbonylamino)ethoxy)-5-(1-methylpyrazol-4-yl)phenyl,
3-(2-(3R—N,N-dimethylaminopyrrolidin-1-ylmethylcarbonylamino)ethoxy)-5-(1-methylpyrazol-4-yl)phenyl,
3-(2-(3S—N,N-dimethylaminopyrrolidin-1-ylmethylcarbonylamino)ethoxy)-5-(1-methylpyrazol-4-yl)phenyl,
3-(2-(4-hydroxypiperidin-1-ylaminomethylcarbonyl)ethoxy)-5-(1-methylpyrazol-4-yl)phenyl,
3-(2-(4-fluoropiperidin-1-ylaminomethylcarbonyl)ethoxy)-5-(1-methylpyrazol-4-yl)phenyl,
3-(2-(N-morpolino)pyridin-5-ylmethoxy)-5-(1-methylpyrazol-4-yl)phenyl,
3-(6-hydroxymethylpyridin-2-ylmethoxy)-5-(1-methylpyrazol-4-yl)phenyl,
3-(2-(2S-methoxycarbonylpyrrolidin-4R-yloxy)-5-(1-methylpyrazol-4-yl)phenyl,
3-(2-(2S-methoxycarbonylpyrrodin-4S-yloxy)-5-(1-methylpyrazol-4-yl)phenyl,
3-(2-(2S-carboxypyrrolidin-4R-yloxy)-5-(1-methylpyrazol-4-yl)phenyl,
3-(2-(2S-carboxypyrrolidin-4S-yloxy)-5-(1-methylpyrazol-4-yl)phenyl,
3-(2-(2S—N-methylaminocarbonylpyrrolidin-4R-yloxy)-5-(1-methylpyrazol-4-yl)phenyl,
3-(242 S—N-methyl aminocarbonylpyrrolidin-4S-yloxy)-5-(1-methylpyrazol-4-yl)phenyl,
3-(2-(2S—N-(2-methoxyethyl)aminocarbonylpyrrolidin-4R-yloxy)-5-(1-methylpyrazol-4-yl)phenyl,
3-(2-(2S—N-(2-methoxyethyl)aminocarbonylpyrrolidin-4S-yloxy)-5-(1-methylpyrazol-4-yl)phenyl,
3-(2-(2-aminothiazol-4-ylmethylcarbonylamino)ethoxy)-5-(1-methylpyrazol-4-yl)phenyl,
3-(2-(thiazol-4-ylcarbonylamino)ethoxy)-5-(1-methylpyrazol-4-yl)phenyl,
3-(2-(2-aminothiazol-5-ylcarbonylamino)ethoxy)-5-(1-methylpyrazol-4-yl)phenyl,
3-(2-(2-aminothiazol-4-ylcarbonylamino)ethoxy)-5-(1-methylpyrazol-4-yl)phenyl,
3-(2-(2-N-acetylaminothiazol-4-ylcarbonylamino)ethoxy)-5-(1-methylpyrazol-4-yl)phenyl,
3-(2-(tetrahydropyran-4-ylamino)ethoxy)-5-(1-methylpyrazol-4-yl)phenyl,
3-(2-(1-methylpiperidin-4-ylamino)ethoxy)-5-(1-methylpyrazol-4-yl)phenyl,
3-(2-hydroxymethyl-3-hydroxypropoxy)-5-(1-methylpyrazol-4-yl)phenyl,
3-(2R-(cyclopropylsulfonylamino)propoxy)-5-(1-methylpyrazol-4-yl)phenyl,
3-(octahydropyrrolo[1,2-a]pyrazin-2-ylcarbonylaminomethoxy)-5-(1-methylpyrazol-4-yl)phenyl,
3-(aminosulfonyl)-5-(aminocarbonyl)phenyl,
3-(N-methyl aminosulfonyl)-5-(N-methylaminocarbonyl)phenyl,
and
3-(2-(2-hydroxypyridin-5-ylcarbonylamino)ethoxy)-5-(1-methylpyrazol-4-yl)phenyl.

In some embodiments, the invention provides compounds that have the Formula I:

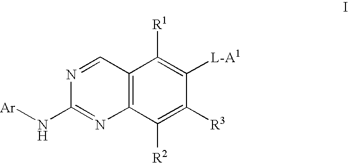

or a pharmaceutically acceptable salt thereof, wherein:

Ar is aryl, substituted aryl, heteroaryl, or substituted heteroaryl, including fused bicyclic systems;

R¹ is H, C₁₋₃ alkyl, halo, cyano, nitro, CF₃, imidazolyl, thiazolyl, oxazolyl, or amino;

R² and R³ are independently selected from the group consisting of H, alkoxy, substituted alkoxy, and halo;

L is a covalent bond, carbonyl, carbonylamino, aminocarbonyl, —O—, —S—, —SO—, —SO₂—, —NH—, C₁₋₃ alkyl, substituted C₁₋₃ alkyl, or an alkyl interrupted with —O—, —S—, —SO—, —SO₂—,

—NH—, carbonyl, carbonylamino, or aminocarbonyl; and

A¹ is alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, sulfonylamino, thioacyl, thiol, alkylthio, substituted alkylthio, aryl, substituted aryl, heteroaryl, substituted heteroaryl, cycloalkyl, substituted cycloalkyl, heterocyclyl, or substituted heterocyclyl.

According to some embodiments, compounds of the invention have Formulae II-VII:

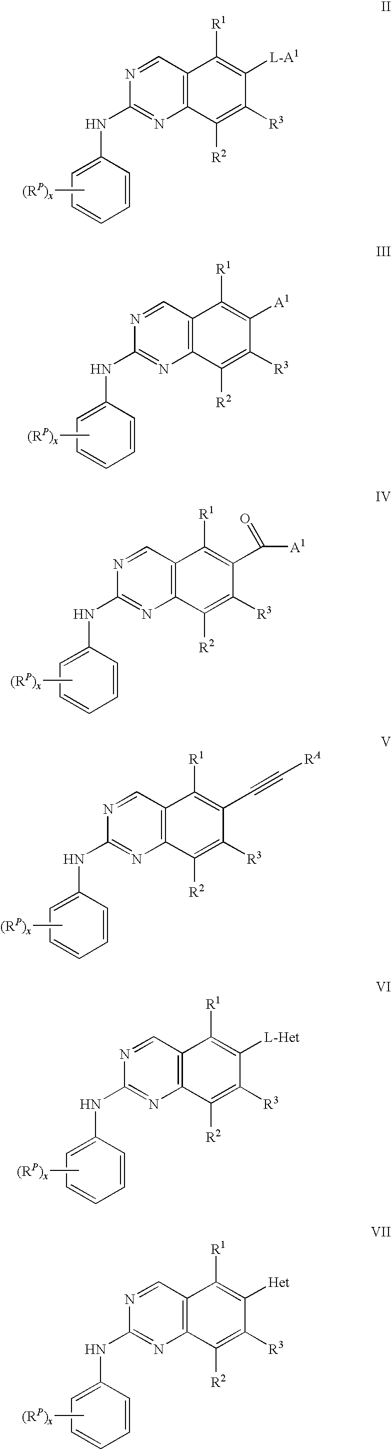

wherein R^{P} is independently selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, to cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio;

R^{A} is selected from the group consisting of H, alkyl, substituted alkyl, aryl, substituted aryl, heteroaryl, substituted heteroaryl, cycloalkyl, substituted cycloalkyl, heterocyclyl and substituted heterocyclyl;

Het is selected from the group consisting of heteroaryl, substituted heteroaryl, heterocyclyl and substituted heterocyclyl; and

x is 1, 2, 3, 4 or 5.

According to some embodiments, compounds of the invention have Formula II:

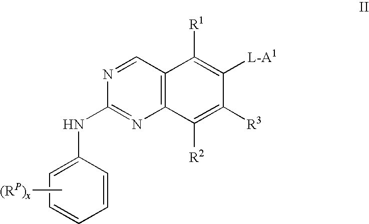

wherein R^{P} is independently selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, alkenyl, alkynyl, aryl, cycloalkyl, cycloalkenyl, heterocyclyl, and heteroaryl moieties contained within any of the preceding “substituted aryl” groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio;

x is 1, 2, 3, 4 or 5; and

R¹, R², R³, L and A¹ are as defined above.

In some embodiments of Formula II, x is 1, 2 or 3; and the isolated phenyl ring in Formula II has no substituents ortho to the NH to which it is attached. Preferred substitution patterns for this phenyl ring include mono-substitution at the 3-position (‘meta’ to the NH); mono-substitution at the 4-position (‘para’ to the NH); and disubstitution at the 3 and 4 positions or at the 3 and 5 positions.

In certain embodiments of the compounds of Formula II, R¹ is H.

In certain embodiments of the compounds of Formula II, R² is substituted alkoxy.

In certain embodiments of the compounds of Formula II, R² is substituted alkoxy, heterocyclyloxy, or heterocyclylalkoxy. In other embodiments, R² is H.

In some embodiments of Formula II, R³ is substituted alkoxy such as heteroarylmethoxy. Suitable heteroaryl groups in these compounds include pyrazole, imidazole, thiazole, pyridine, and pyrazole and pyrimidine. In other such embodiments, R³ is heterocyclyloxy or heterocyclyl-substituted alkoxy such as heterocyclylmethoxy. In other such embodiments, R³ is heterocyclyl-substituted alkoxy such as heterocyclylmethoxy. Suitable heterocyclyl groups for these embodiments include piperidinyl, pyrrolidinyl, tetrahydrofuranyl, and the like.

In some embodiments, at least one R^{P} present comprises a heteroaryl or heterocyclic group. In some embodiments, it is a heteroaryl group, which may be substituted. Suitable heteroaryls include pyridinyl, imidazolyl, pyrazolyl, pyrimidinyl, thiazolyl, triazolyl, tetrazolyl, oxazolyl, thiazolyl, and thiadiazolyl. In others, R^{P} is a group of the formula —O—CH₂—C(O)—NR′R″, where R¹ and R″ are independently H, alkyl, or substituted alkyl, and R′ and R″ can join together to form a heterocyclic ring. In other embodiments, R^{P} is a heterocyclyl group such as piperazinyl, piperidinyl, morpholinyl, or R^{P} is a heterocyclyl-substituted alkyl such as piperazinylmethyl, morpholinylmethyl, oxazolinylmethyl, and the like. In some embodiments, R^{P} is a heterocyclyl or heteroaryl group linked to the phenyl ring of Formula II through —O— or OCH₂— or —OCH₂—CH₂—.

According to some embodiments, compounds of the invention have Formula III:

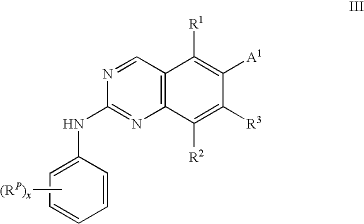

wherein R¹, R², R³, A¹, R^{P} and x are as defined above. These correspond to compounds wherein L is a bond. Typically in these embodiments, at least one of R¹, R² and R³ is a group other than H. Frequently, R¹ is H or halo, and either R² or R³ is H while the other of R² and R³ is a group selected from alkoxy, substituted alkoxy, heteroaryloxy, substituted heteroaryloxy, heterocyclyloxy, and substituted heterocyclyloxy.

According to some embodiments, compounds of the invention have Formula IV:

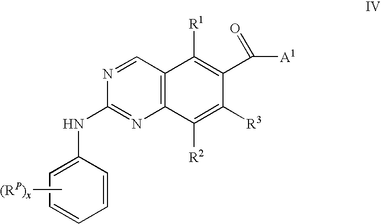

wherein R¹, R², R³, A¹, R^{P} and x are as defined above.

According to some embodiments, compounds of the invention have Formula V:

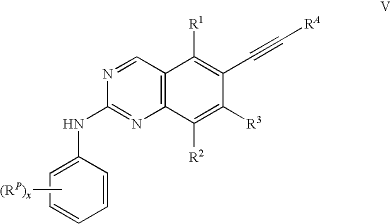

wherein R¹, R², R³, R^{P} and x are as defined above; and

R^{A} is selected from the group consisting of H, alkyl, substituted alkyl, aryl, substituted aryl, heteroaryl, substituted heteroaryl, cycloalkyl, substituted cycloalkyl, heterocyclyl and substituted heterocyclyl. Frequently, R^{A} is selected from H, methyl, hydroxymethyl, methoxymethyl, and other alkoxymethyl groups. Often in these compounds, R¹, R², R³, R^{P} and x are as described for the compounds of Formula II above.

According to some embodiments, compounds of the invention have Formula VI:

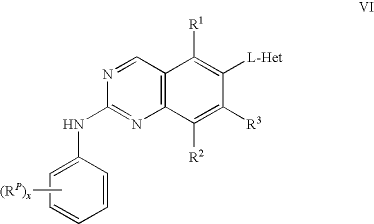

wherein R¹, R², R³, L, R^{P} and x are as defined above; and

Het is selected from the group consisting of heteroaryl, substituted heteroaryl, heterocyclyl and substituted heterocyclyl. Often in these compounds, R¹, R², R³, R^{P} and x are as described for the compounds of Formula II above. In some of these embodiments, L is a bond, —O—, —OCH₂—, amino, aminocarbonyl, or carbonylamino.

According to some embodiments, compounds of the invention have Formula VII:

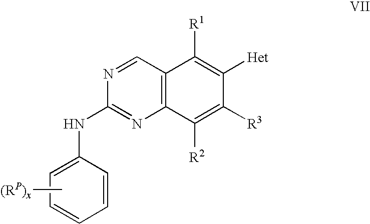

wherein R¹, R², R³, R^{P}, x and Het are as defined above. Often in these compounds, R¹, R², R³, R^{P} and x are as described for the compounds of Formula II above. Het in these compounds can be any heterocyclic or heteroaryl group, and sometimes it is selected from thiazole, oxazole, isothiazole, isoxazole, pyrazole, pyridine, triazole, and furan.

In some embodiments of the compounds of Formula I, Ar is phenyl substituted by a heterocycloalkylalkyl group which is substituted by 1 or 2 groups independently selected from hydroxyl, halogen, cyano, C₁₋₆ alkyl, C₁₋₆ haloalkyl, amino, C₁₋₄-alkylamino, di-C₁₋₄-alkylamino, carboxy, C₁₋₆ alkoxycarbonyl, C₁₋₆ alkylcarbonyl, carbamyl, C₁₋₄-alkylcarbamyl, and di-C₁₋₄-alkylcarbamyl; where said C₁₋₆ alkyl, C₁₋₆ haloalkyl, C₁₋₄-alkylamino, di-C₁₋₄-alkylamino, C₁₋₆ alkoxycarbonyl, C₁₋₆ alkylcarbonyl, C₁₋₄-alkylcarbamyl, and di-C₁₋₄-alkylcarbamyl are each optionally substituted by 1 or 2 groups independently selected from C₁₋₄ alkyl, hydroxyl, C₁₋₄ alkoxy, amino, C₁₋₄ alkylamino, di-C₁₋₄-alkylamino, 5-membered heterocycloalkyl, and 6-membered heterocycloalkyl; provided that the compound is not selected from any one of the compounds of Tables 2, 3, and 4 or in the examples section from PCT/US2007/088392.

In some embodiments of the compounds of Formula I, -L-A¹ is a —O-heterocycloalkyl moiety, which is substituted by 1 or 2 groups independently selected from halogen, C₁₋₆ alkyl, C₁₋₆ haloalkyl, hydroxyl, C₁₋₆ alkoxy, amino, C₁₋₆ alkylamino, di-C₁₋₄-alkylamino, carboxy, C₁₋₆ alkoxycarbonyl, carbamyl, C₁₋₆ alkylcarbamyl, and di-C₁₋₄-alkylcarbamyl; wherein said C₁₋₆ alkyl, C₁₋₆ haloalkyl, C₁₋₆ alkoxy, C₁₋₆ alkylamino, di-C₁₋₄-alkylamino, C₁₋₆ alkoxycarbonyl, C₁₋₆ alkylcarbamyl, and di-C₁₋₄-alkylcarbamyl are each optionally substituted by 1 or 2 groups independently selected from hydroxyl and amino; provided that the compound is not selected from any one of the compounds of Tables 2, 3, and 4 or in the examples section from PCT/US2007/088392.

In some embodiments, the compound of Formula (Ia), (IIa), (IIIa), and (IVa), or any embodiment thereof described herein, is not selected from any one of the compounds of Tables 2, 3, and 4. In some embodiments, the compound of Formula I, or any embodiment described herein, is not selected from any one of the compounds of Tables 2, 3, and 4. In some embodiments, the compound of Formula (Ia), (IIa), (IIIa), and (IVa), or any embodiment thereof described herein, is not selected from any one of the compounds of PCT/US2007/088392, e.g., as described in Tables 2, 3, and 4 or in the examples section from PCT/US2007/088392, including the intermediates (included below). In some embodiments, the compound of Formula I, or any embodiment thereof described herein, is not selected from any one of the compounds of PCT/US2007/088392, e.g., as described in Tables 2, 3, and 4 or in the examples section from PCT/US2007/088392, including the intermediates (included below). Tables 2, 3, and 4 show the activity for each of the compounds of PCT/US2007/088392 as well. The column marked “Activity” indicates the compound's activity in the PDK1 Kinase Alpha Screen Assay described below. The symbol “+” indicates IC₅₀ values of 25 μm or greater (or compounds not evaluated), the symbol “++” indicates IC₅₀ values between less than 25 μm and greater than 10 μm, the symbol “+++” indicates IC₅₀ values of 10 μm or less and greater than 5 μm, and the symbol “++++” indicates IC₅₀ values less than 5 p.m.

The compounds in this invention may contain one or more asymmetric centers, which can thus give rise to optical isomers (enantiomers) and diastereomers. While shown without respect to the stereochemistry in Formula I, the present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. The use of these compounds is intended to cover the racemic mixture or either of the chiral enantiomers.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

One skilled in the art will also recognize that it is possible for tautomers to exist for the compounds of the present invention. The present invention includes all such tautomers even though not shown in the formulas herein.

It is further intended that the compounds of the invention include hydrated, solvated, anhydrous, and non-solvated forms.

Compounds of the invention can also include different atomic isotopes. Isotopes include those atoms having the same atomic number but different mass numbers. For example, isotopes of hydrogen include tritium and deuterium. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention, tautomers thereof, prodrugs thereof, and pharmaceutically acceptable salts of the compounds and of the prodrugs that contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out known or referenced procedures and by substituting a readily available isotopically labeled reagent for a non-isotopically labeled reagent.

In some embodiments, the compounds of the invention, and their salts, esters, tautomers, etc., are isolated. By “isolated” or “substantially isolated” is meant that the compound is at least partially or substantially separated from the environment in which is was formed or discovered. Partial separation can include, for example, a composition enriched in the compound of the invention. Substantial separation can include compositions containing at least about 50%, at least about 60%, at least about 70%, at least about 80%, at least about 90%, at least about 95%, and at least about 99% by weight of the compound of the invention, or salt thereof. Methods for isolating compounds and their salts and other derivatives are routine in the art.

The compounds of the present invention also include pharmaceutically acceptable salts of the compounds disclosed herein. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi-salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties. In some embodiments, the term “pharmaceutically acceptable salt” refers to a quaternary salt of an basic nitrogen atom (such as that in a morpholine ring).

In some embodiments, the compounds of the invention are prodrugs. As used herein, “prodrug” refers to a moiety that releases a compound of the invention when administered to a patient. Prodrugs can be prepared by modifying functional groups present in the compounds in such a way that the modifications are cleaved, either in routine manipulation or in vivo, to the parent compounds. Examples of prodrugs include compounds of the invention as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R. In another example, when an optionally multiple substituent is designated in the form:

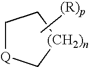

then it is understood that substituent R can occur p number of times on the ring, and R can be a different moiety at each occurrence. It is understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

Throughout the definitions, the term “Cₙ₋ₘ” is referred to indicate C₁₋₄, C₁₋₆, and the like, wherein n and m are integers and indicate the number of carbons, wherein n-m indicates a range which includes the endpoints.

Unless indicated otherwise, the nomenclature of substituents that are not explicitly defined herein are arrived at by naming the terminal portion of the functionality followed by the adjacent functionality toward the point of attachment. In generally, the point of attachment for a substituent is indicated by the last term in the group. For example, C₁₋₆ heteroaryl-C₁₋₄ alkyl refers to a moiety of heteroaryl-alkylene-, wherein the heteroaryl group has 1 to 6 carbon atoms, the alkylene linker has 1 to 4 carbons, and the substituent is attached through the alkylene linker.

As used herein, the phrase “optionally substituted” means unsubstituted or substituted. As used herein, the term “substituted” means that a hydrogen atom is removed and replaced by a substituent. As used herein, the phrase “substituted with oxo” means that two hydrogen atoms are removed from a carbon atom and replaced by an oxygen bound by a double bond to the carbon atom. It is understood that substitution at a given atom is limited by valency.

The Definitions that Follow Apply to the Terms in the Variable Definitions for Each Substituent for the Compounds of Formula (Ia), (IIa), (IIIa), and (IVa):

As used herein, the term “Cₙ₋ₘ alkyl”, employed alone or in combination with other terms, refers to a saturated hydrocarbon group that may be straight-chain or branched, having n to m carbons. In some embodiments, the alkyl group contains from 1 to 7 carbon atoms, from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like.

As used herein, the term “Cₙ₋ₘ alkylamino”, employed alone or in combination with other terms, refers to a group of formula NH(alkyl), wherein the alkyl group has n to m carbon atoms.

As used herein, the term “Cₙ₋ₘ alkylcarbamyl”, employed alone or in combination with other terms, refers to a group of formula C(O)—NH(alkyl), wherein the alkyl group has n to m carbon atoms.

As used herein, the term “Cₙ₋ₘ alkylene”, employed alone or in combination with other terms, refers to a divalent alkyl linking group having n to m carbon atoms. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

As used herein, “Cₙ₋ₘ alkynyl”, employed alone or in combination with other terms, refers to an alkyl group having one or more triple carbon-carbon bonds with n to m carbon atoms. Example alkynyl groups include, but are not limited to, ethynyl, propyn-1-yl, propyn-2-yl, and the like. In some embodiments, the alkynyl moiety contains 2 to 10 or 2 to 6 carbon atoms.

As used herein, the term “Cₙ₋ₘ alkynylene”, employed alone or in combination with other terms, refers to a divalent alkynyl group having n to m carbon atoms. In some embodiments, the alkynylene moiety contains 2 to 12 carbon atoms. In some embodiments, the alkynylene moiety contains 2 to 6 carbon atoms. Example alkynylene groups include, but are not limited to, ethyn-1,2-diyl, propyn-1,3,-diyl, 1-butyn-1,4-diyl, 1-butyn-1,3-diyl, 2-butyn-1,4-diyl, and the like.

As used herein, the term “Cₙ₋ₘ alkoxy”, employed alone or in combination with other terms, refers to an group of formula —O-alkyl, wherein the alkyl group has n to m carbon atoms. Example alkoxy groups include methoxy, ethoxy, propoxy (e.g., n-propoxy and isopropoxy), t-butoxy, and the like.

As used herein, the term “Cₙ₋ₘ alkoxycarbonyl”, employed alone or in combination with other terms, refers to a group of formula —C(O)O-alkyl, wherein the alkyl group has n to m carbon atoms.

As used herein, the term “Cₙ₋ₘ alkylcarbonyl”, employed alone or in combination with other terms, refers to a group of formula —C(O)-alkyl, wherein the alkyl group has n to m carbon atoms.

As used herein, the term “Cₙ₋ₘ alkylcarbonylamino”, employed alone or in combination with other terms, refers to a group of formula —NHC(O)-alkyl, wherein the alkyl group has n to m carbon atoms.

As used herein, the term “alkylaminosulfonyl”, employed alone or in combination with other terms, refers to a group of formula —S(O)₂NH-alkyl.

As used herein, the term “aminosulfonyl”, employed alone or in combination with other terms, refers to a group of formula —S(O)₂NH₂.

As used herein, the term “amino”, employed alone or in combination with other terms, refers to a group of formula NH₂.

As used herein, the term “Cₒ₋ₚ heteroaryl-Cₙ₋ₘ-alkyl”, employed alone or in combination with other terms, refers to a group of formula -alkynylene-heteroaryl, wherein the alkynylene linker has n to m carbon atoms.

As used herein, the term “carbamyl”, employed alone or in combination with other terms, refers to a group of formula C(O)NH₂.

As used herein, the term “carbonyl”, employed alone or in combination with other terms, refers to a —C(O)— group, which is a divalent one-carbon moiety further bonded to an oxygen atom with a double bond.

As used herein, the term “carboxy”, employed alone or in combination with other terms, refers to a group of formula C(O)OH.

As used herein, the term “cycloalkyl”, employed alone or in combination with other terms, refers to a non-aromatic cyclic hydrocarbon moiety, which may optionally contain one or more alkenylene or alkynylene groups as part of the ring structure. Cycloalkyl groups can include mono- or polycyclic (e.g., having 2, 3 or 4 fused or covalently linked rings) ring systems. Also included in the definition of cycloalkyl are moieties that have one or more aromatic rings fused (i.e., having a bond in common with) to the cycloalkyl ring, for example, benzo derivatives of pentane, pentene, hexane, and the like. The term “cycloalkyl” also includes bridgehead cycloalkyl groups and spirocycloalkyl groups. As used herein, “bridgehead cycloalkyl groups” refers to non-aromatic cyclic hydrocarbon moieties containing at least one bridgehead carbon, such as admantan-1-yl. As used herein, “spirocycloalkyl groups” refers to non-aromatic hydrocarbon moieties containing at least two rings fused at a single carbon atom, such as spiro[2.5]octane and the like. In some embodiments, the cycloalkyl group has 3 to 14 ring members, 3 to 10 ring members, or 3 to 8 ring members. One or more ring-forming carbon atoms of a cycloalkyl group can be oxidized to form carbonyl linkages. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, the cycloalkyl group is admanatan-1-yl.

As used herein, the term “cyano”, employed alone or in combination with other terms, refers to a group of formula —CN, wherein the carbon and nitrogen atoms are bound together by a triple bond.

As used herein, the term “di-Cₙ₋ₘ-alkylamino”, employed alone or in combination with other terms, refers to a group of formula —N(alkyl)₂, wherein the alkylene group and two alkyl groups each has, independently, n to m carbon atoms.

As used herein, “haloalkoxy”, employed alone or in combination with other terms, refers to a group of formula —O-haloalkyl. An example haloalkoxy group is OCF₃.

As used herein, the term “haloalkyl”, employed alone or in combination with other terms, refers to an alkyl group having from one halogen atom to 2n+1 halogen atoms which may be the same or different, where “n” is the number of carbon atoms in the alkyl group. In some embodiments, the halogen atoms are fluoro atoms.

As used herein, the terms “halo” and “halogen”, employed alone or in combination with other terms, refer to fluoro, chloro, bromo, and iodo.

As used herein, the term “heteroaryl”, “heteroaryl ring”, or “heteroaryl group”, employed alone or in combination with other terms, refers to a monocyclic or polycyclic (e.g., having 2, 3 or 4 fused or covalently linked rings) aromatic hydrocarbon moiety, having one or more heteroatom ring members selected from nitrogen, sulfur and oxygen.

When the heteroaryl group contains more than one heteroatom ring member, the heteroatoms may be the same or different. Example heteroaryl groups include, but are not limited to, pyrrolyl, azolyl, oxazolyl, thiazolyl, imidazolyl, furyl, thienyl, quinolinyl, isoquinolinyl, indolyl, benzothienyl, benzofuranyl, benzisoxazolyl, imidazo[1,2-b]thiazolyl or the like. In some embodiments, the heteroaryl group has 5 to 10 carbon atoms.

As used herein, the term “heteroaryl-Cₙ₋ₘ-alkyl”, employed alone or in combination with other terms, refers to a group of formula alkylene-heteroaryl, the alkylene linker has n to m carbon atoms. In some embodiments, the alkyl portion of the heteroaryl group has 1 to 4 carbon atoms.

As used herein, the term “Cₒ₋ₚ, heteroaryl-Cₙ₋ₘ-alkoxy”, employed alone or in combination with other terms, refers to a group of formula —O-alkylene-heteroaryl, the alkylene linker has n to m carbon atoms and the heteroaryl has o to p carbon atoms. In some embodiments, the alkyl portion of the heteroaryl group has 1 to 4 carbon atoms.

As used herein, the term “heteroaryloxy”, employed alone or in combination with other terms, refers to a group of formula —O-heteroaryl.

As used herein, the term “heterocycloalkyl”, “heterocycloalkyl ring”, or “heterocycloalkyl group”, employed alone or in combination with other terms, refers to non-aromatic ring system, which may optionally contain one or more alkenylene or alkynylene groups as part of the ring structure, and which has at least one heteroatom ring member selected from nitrogen, sulfur and oxygen. When the heterocycloalkyl groups contains more than one heteroatom, the heteroatoms may be the same or different. Heterocycloalkyl groups can include mono- or polycyclic (e.g., having 2, 3 or 4 fused or covalently bonded rings) ring systems. Also included in the definition of heterocycloalkyl are moieties that have one or more aromatic rings fused (i.e., having a bond in common with) to the non-aromatic ring, for example, 1,2,3,4-tetrahydro-quinoline and the like. Heterocycloalkyl groups can also include bridgehead heterocycloalkyl groups and spiroheterocycloalkyl groups. As used herein, “bridgehead heterocycloalkyl group” refers to a heterocycloalkyl moiety containing at least one bridgehead atom, such as azaadmantan-1-yl and the like. As used herein, “spiroheterocycloalkyl group” refers to a heterocycloalkyl moiety containing at least two rings fused at a single atom, such as [1,4-dioxa-8-aza-spiro[4.5]decan-N-yl] and the like. In some embodiments, the heterocycloalkyl group has 3 to 20 ring-forming atoms, 3 to 10 ring-forming atoms, or about 3 to 8 ring forming atoms. The carbon atoms or hetereoatoms in the ring(s) of the heterocycloalkyl group can be oxidized to form a carbonyl, or sulfonyl group (or other oxidized linkage) or a nitrogen atom can be quaternized.

Where a particular heteraryl or heterocycloalkyl group appears in the embodiments, such as “a pyrazole ring”, the term is intended to refer to a pyrazole ring attached at any atom of the ring, as permitted by valency rules, and is intended to include various tautomeric forms of the ring. Conversely, in some embodiments, the point of attachment is indicated by the name, e.g., pyrazol-1-yl refers to a pyrazole ring attached at the 1-position of the ring.

As used herein, the term “heterocycloalkyl-Cₙ₋ₘ-alkyl”, employed alone or in combination with other terms, refers to a group of formula alkylene-heterocycloalkyl, the alkylene linker has n to m carbon atoms. In some embodiments, the alkyl portion of the heterocycloalkylalkyl group has 1 to 4 carbon atoms. In some embodiments, the alkyl portion of the heterocycloalkylalkyl group is methylene. In some embodiments, the heterocycloalkylalkyl group is (tetrahydrofur-2-yl)methyl.

As used herein, the term “heterocycloalkyloxy”, employed alone or in combination with other terms, refers to a group of formula —O-heterocycloalkyl.

As used herein, the term “hydroxyl”, employed alone or in combination with other terms, refers to a group of formula —OH.

The Definitions that Follow Apply to the Terms in the Variable Definitions for Each Substituent for the Other Compounds Described Herein:

“Aryl” or refers to a monovalent aromatic carbocyclic group of from 6 to 14 carbon atoms having a single ring or multiple condensed rings. Non-limiting examples of aryl groups include phenyl, naphthyl, anthryl, and the like.

“Substituted aryl” refers to aryl groups which are substituted with 1 to 5, or 1 to 3, or 1 to 2 substituents selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, alkenyl, alkynyl, aryl, cycloalkyl, cycloalkenyl, heterocyclyl, and heteroaryl moieties contained within any of the preceding “substituted aryl” groups are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio; and wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Alkyl” refers to monovalent saturated aliphatic hydrocarbyl groups having from 1 to 10 carbon atoms or from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, or 1 to 3, or 1 to 2 substituents selected from the group consisting of alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, aryl, cycloalkyl, cycloalkenyl, heterocyclyl, and heteroaryl moieties contained within any of the preceding “substituted alkyl” groups, are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio; and wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups NRC(O)alkyl, —NRC(O) substituted alkyl, —NRC(O)cycloalkyl, —NRC(O) substituted cycloalkyl, —NRC(O)cycloalkenyl, —NRC(O) substituted cycloalkenyl, —NRC(O)alkenyl, —NRC(O) substituted alkenyl, —NRC(O)alkynyl, —NRC(O) substituted alkynyl, —NRC(O)aryl, —NRC(O) substituted aryl, —NRC(O)heteroaryl, —NRC(O) substituted heteroaryl, —NRC(O)heterocyclic, and —NRC(O) substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkenyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group NR′R″ where R¹ and R″ are independently selected from the group consisting of hydrogen, alkyl, alkenyl, alkynyl, aryl, cycloalkyl, cycloalkenyl, heteroaryl, heterocyclic, —SO₂-alkyl, —SO₂-alkenyl, —SO₂-cycloalkyl, —SO₂-cycloalkenyl, —SO₂-aryl, —SO₂-heteroaryl, and —SO₂-heterocyclic,

wherein R¹ and R″ are optionally joined, together with the nitrogen bound thereto to form a heterocyclic or substituted heterocyclic group, provided that R′ and R″ are both not hydrogen;

wherein the alkyl, alkenyl, alkynyl, aryl, cycloalkyl, cycloalkenyl, heterocyclyl, and heteroaryl moieties contained within any of the preceding “substituted amino” groups are optionally substituted by 1, 2, 3, 4 or 5 substituents independently selected from the group consisting of alkenyl, alkynyl, alkoxy, acyl, acylamino, acyloxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, and alkylthio;

and wherein said substituents are defined herein.

When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —OC(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Amidino” refers to the group —C(═NR¹²)R¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Alkenyl” refers to alkenyl groups having from 2 to 6 carbon atoms or from 2 to 4 carbon atoms and having at least 1 and in some embodiments, from 1 to 2 sites of alkenyl unsaturation. Such groups are exemplified, for example, by vinyl, allyl, and but-3-en-1-yl.

“Substituted alkenyl” refers to alkenyl groups having from 1 to 3 substituents, or from 1 to 2 substituents, selected from the group consisting of alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, thioacyl, thiol, and alkylthio,

wherein the alkyl, aryl, cycloalkyl, cycloalkenyl, heterocyclyl, and heteroaryl moieties contained within any of the preceding “substituted alkenyl” groups are optionally substituted by 1, 2, 3, 4 or 5 substituents independently selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, and alkylthio; wherein said substituents are defined herein and with the proviso that any hydroxy substitution is not attached to a vinyl (unsaturated) carbon atom.

“Alkynyl” refers to alkynyl groups having from 2 to 6 carbon atoms or from 2 to 3 carbon atoms and having at least 1 and, in some embodiments, 1 to 2 sites of alkynyl unsaturation.

“Substituted alkynyl” refers to alkynyl groups having from 1 to 3 substituents, and, in some embodiments, 1 to 2 substituents, selected from the group consisting of alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio,

wherein the alkyl, aryl, cycloalkyl, cycloalkenyl, and heteroaryl moieties contained within any of the preceding “substituted alkynyl” are optionally substituted by 1, 2, 3, 4 or 5 substituents independently selected from the group consisting of alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, and alkylthio; wherein said substituents are defined herein and with the proviso that any hydroxy substitution is not attached to an acetylenic carbon atom.

“Carbonyl” refers to the divalent group C(O) which is equivalent to —C(═O)—.

“Carboxyl” or “carboxy” refers to COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, substituted —NR—C(O)O-alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiroring systems. Cycloalkyl groups can include. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation and, in some embodiments, from 1 to 2 sites of >C═C< ring unsaturation.

“Substituted cycloalkyl” and “substituted cycloalkenyl” refers to a cycloalkyl or cycloalkenyl group having from 1 to 5 and, in some embodiments, 1 to 3 substituents selected from the group consisting of oxo, thione, alkyl, alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, substituted sulfonyl, sulfonyloxy, sulfonylamino, thioacyl, thiol, and alkylthio;

wherein the alkyl, alkenyl, alkynyl, aryl, cycloalkyl, cycloalkenyl, heterocyclyl, and heteroaryl moieties contained within any of the preceding “substituted cycloalkyl” or “substituted cycloalkenyl” groups, are optionally substituted by 1, 2, 3, 4 or 5 substituents independently selected from the group consisting of alkenyl, alkynyl, alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyl, cycloalkyloxy, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, alkylthio, and alkylthio; and wherein said substituents are defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, aryl, heteroaryl, heterocyclic, and two R¹³ groups attached to a common guanidino nitrogen atom are optionally joined together with the nitrogen bound thereto to form a heterocyclic or substituted heterocyclic group, provided that at least one R¹³ is not hydrogen;

wherein the alkyl, aryl, heterocyclyl, and heteroaryl moieties contained within any of the preceding “substituted guanidino” groups are optionally substituted by 1, 2, 3, 4 or 5 substituents independently selected from the group consisting of alkenyl, alkynyl, alkoxy, acyl, acylamino, acyloxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, carboxyl, carboxyl ester, (carboxyl ester)amino, (carboxyl ester)oxy, cyano, cycloalkyl, cycloalkyloxy, cycloalkylthio, cycloalkenyl, cycloalkenyloxy, cycloalkenylthio, guanidino, substituted guanidino, halo, hydroxy, heteroaryl, heteroaryloxy, heteroarylthio, heterocyclic, heterocyclyloxy, heterocyclylthio, nitro, SO₃H, sulfonyl, sulfonyloxy, sulfonylamino, thioacyl, thiol, and alkylthio.

“Halo” or “halogen” refers to fluoro, chloro, bromo and iodo.

“Hydroxy” or “hydroxyl” refers to the group OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can be monocyclic, i.e., have a single ring (e.g., pyridinyl or furyl) or polycyclic, i.e., having multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Monocyclic heteroaryls include without limitation, pyrrolyl, furanyl, thiophenyl (thienyl), imidazolyl, pyrazolyl, oxazolyl, isoxazolyl, thiazolyl, isothiazolyl, 1,2,3-triazolyl, 1,2,4-triazolyl, oxadiazolyl, thiadiazolyl, tetrazolyl, pyridinyl, pyrazinyl, pyrimidinyl, pyridazinyl, triazinyl and the like. Polycyclic heteroaryls include without limitation, indolyl, isoindolyl, benzimidazolyl, benzothiophenyl, benzofuranyl, benzoxazolyl, benzothiazolyl, quinolinyl, isoquinolyl, quinazolyl, quinozalyl, cinnolyl, pteridine, carbazole, carboline, phenanthridine, acridine, phenanthroline, phthalazine, naphthylpyridine, phenazine, purine and the like.

“Substituted heteroaryl” refers to heteroaryl groups that are substituted with from 1 to 5, and, in some embodiments, 1 to 3, and, in some embodiments, 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a non-aromatic heterocycle where one or more of the ring-forming atoms is a heteroatom such as an O, N, or S atom. Heterocycloalkyl groups can include mono- or polycyclic (e.g., having 2, 3 or 4 fused rings) ring systems as well as spirocycles. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. A heterocycloalkyl group having one or more fused aromatic rings can be attached though either the aromatic or non-aromatic portion. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds. In some embodiments, the nitrogen and/or sulfur atom(s) of the heterocyclic group are optionally oxidized to provide for the N-oxide, sulfoxide, and sulfone moieties.

“Substituted heterocyclic” or “substituted heterocycloalkyl” or “substituted heterocyclyl” refers to heterocyclyl groups that are substituted with from 1 to 5 and, in some embodiments, 1 to 3 of the same substituents as defined for substituted cycloalkyl.

“Heterocyclyloxy” refers to the group —O-heterocyclyl.

“Substituted heterocyclyloxy refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycles include, but are not limited to, azetidine, indolizine, dihydroindole, indazole, quinolizine, isothiazole, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazolidine, morpholinyl, thiomorpholinyl (also referred to as thiamorpholinyl), 1,1-dioxothiomorpholinyl, piperidinyl, pyrrolidine, tetrahydrofuranyl, and the like.

“Nitro” refers to the group —NO₂.

“Oxo” refers to the atom (═O) or (—O—).

“Spirocycloalkyl” refers to divalent cyclic groups from 3 to 10 carbon atoms having a cycloalkyl ring with a —Spiro union (the union formed by a single atom which is the only common member of the rings) as exemplified by the following structure:

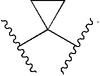

“Sulfonyl” or “sulfone” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —O—SO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cyclo-alkenyl-C(S)—, aryl-C(S)—, substituted aryl-C(S)—, heteroaryl-C(S)—, substituted hetero-aryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thiol” refers to the group —SH.

“Thiocarbonyl” refers to the divalent group —C(S)— which is equivalent to —C(═S)—

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

The expression “an alkyl interrupted with —O—, —S—, —SO—, —SO₂—, —NH—, carbonyl, carbonylamino, or aminocarbonyl” refers to an alkyl group wherein one divalent carbon unit, i.e., a methylene (—CH₂—) in the alkyl group is replaces by one of the listed divalent moieties.

Other Definitions

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or ACN are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

In accordance with further embodiments of the present invention, therapeutic compositions are provided. Such compositions include a therapeutically effective amount of a compound of the invention (i.e., a compound of Formula I) and at least one pharmaceutically acceptable carrier. Accordingly, the present invention provides a pharmaceutical composition comprising a compound of the invention, or pharmaceutically acceptable salt thereof, and a pharmaceutically acceptable carrier.

Pharmaceutical compositions that include the compounds described herein may include additives such as pharmaceutically acceptable carriers or excipients. Suitable pharmaceutically acceptable carriers include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more of these. Other suitable pharmaceutically acceptable carriers are described in Remington: The Science And Practice Of Pharmacy, Lippincott Williams & Wilkins; Baltimore, Md., 21st ed. (May 28, 2005), which is hereby incorporated herein by reference in its entirety and for all purposes as if fully set forth herein.

Pharmaceutical compositions that include the compounds of the invention may be in any form suitable for the intended method of administration, including, for example, as a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more of these. The liquid carrier may include other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, but are not limited to, soybean oil, coconut oil, olive oil, safflower oil, cottonseed oil, and the like. For parenteral administration, the carrier may be an oily ester such as ethyl oleate, isopropyl myristate, and the like. Compositions of the present invention may also be in the form of microparticles, microcapsules, and the like, as well as combinations of any two or more of these.

The compounds and combinations of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form may include, in addition to a compound of the present invention, stabilizers, preservatives, excipients, and the like. Preferred lipids include phospholipids and phosphatidyl cholines (lecithins), both natural and synthetic. Methods of forming liposomes are known in the art. See, for example, Prescott, Ed., Methods in Cell Biology, Volume XIV, Academic Press, New York, N.W., p. 33 et seq (1976).

Controlled release delivery systems may also be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation spray, rectally, or topically in dosage unit formulations that include conventional nontoxic pharmaceutically acceptable carriers, adjuvants, and vehicles as desired. For example, suitable modes of administration include oral, subcutaneous, transdermal, transmucosal, iontophoretic, intravenous, intramuscular, intraperitoneal, intranasal, subdermal, rectal, and the like. Topical administration may also include the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will, therefore, melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also include, as is normal practice, additional substances other than inert diluents, e.g., lubricating agents such as magnesium stearate. In the case of capsules, tablets, and pills, the dosage forms may also include buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and severity of the particular disease undergoing therapy. The therapeutically effective amount for a given situation can be readily determined by routine experimentation and is within the skill and judgment of the ordinary clinician.

In some embodiments, the compounds of the invention can be administered to a patient in combination with one or more further pharmaceutical agents. Administration of the different agents can be made separately either sequentially or simultaneously, or the agents can be administered together in a single composition. Example further pharmaceutical agents include anti-cancer drugs including chemotherapeutics and other kinase inhibiting compounds.

The therapeutically effective amount provided in the treatment of a specific disorder will vary depending the specific disorder(s) being treated, the size, age, and response pattern of the individual the severity of the disorder(s), the judgment of the attending clinician, the manner of administration, and the purpose of the administration, such as prophylaxis or therapy. In some embodiments, effective amounts for daily oral administration may be about 0.01 to 50 mg/kg, or about 0.1 to 10 mg/kg and effective amounts for parenteral administration may be about 0.01 to 10 mg/kg, preferably about 0.1 to 5 mg/kg.

As used herein, the term “treating” or “treatment” refers to (1) inhibiting a disease; for example, inhibiting a disease, condition or disorder in an individual who is experiencing or displaying the pathology or symptomotology of the disease, condition or disorder; (2) preventing the disease; for example, preventing a disease, condition or disorder in an individual who may be predisposed to the disease, condition or disorder but does not yet experience or display the pathology or symptomatology of the disease; (3) delaying recurrence of the disease, for example, increasing the duration of a period of remission in a proliferative disorder such as a cancer; or (4) ameliorating the disease; for example, ameliorating a disease, condition or disorder in an individual who is experiencing or displaying the pathology or symptomatology of the disease, condition or disorder. Treatment of a patient is typically carried out by administration of a compound of the invention to the patient in a pharmaceutically effective amount.

A “subject,” “individual” or “patient” is meant to describe a human or vertebrate animal including, for example, a dog, cat, horse, cow, pig, sheep, goat, monkey, owl, rat, and mouse. In some embodiments, the “subject,” “individual” or “patient” is human. In further embodiments, the “subject,” “individual” or “patient” is in need of treatment, that is, the patient can be afflicted with, is likely to be afflicted with, or might be afflicted with a disease which is treatable by administration of a compound of the invention, or pharmaceutically acceptable salt, ester, or tautomer thereof, or composition comprising the same.

The compounds of the invention are useful for human or veterinary use where, for example, inhibition of PDK1 or inhibition of PDK1 variants is indicated, such as in the treatment of various diseases associated with abnormal PDK1 signaling and/or abnormal signaling upstream or downstream of PDK1 (or variants thereof), such as that related to up-regulated activity of one or more receptor tyrosine kinases, Ras, PI3K, PDK1, AKT, RSK, PKC, 70S6K, or SGK. In some embodiments, the compounds of the invention are useful in inhibiting PDK1 variants wherein the wild type PDK1 contains one or more point mutations, insertions, or deletions. Example PDK1 variants include as PDK1^{T354M} and PDK1^{D527E}. Accordingly, the present invention provides a method of inhibiting PDK1 or a PDK1 variant in a patient comprising administering to said patient, an effective amount of a compound of the invention, or pharmaceutically acceptable salt thereof.

The term “PDK1” is meant to refer to wild type PDK1. The term “PDK1 variant” or “variant of PDK1” is meant to refer to PDK1 having at least one point mutation, insertion, or deletion.

The compounds of the invention can be used in the treatment of diseases characterized by “abnormal cellular proliferation.” The term “abnormal cellular proliferation” includes, for example, any disease or disorder characterized by excessive or pathologically elevated cell growth such as is characteristic of various cancers and non-cancer proliferative disorders.

Example cancers include, for example, lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, and pharyngeal cancer, laryngeal cancer, small intestinal cancer, non-Hodgkin lymphoma, and villous colon adenoma.

Example non-cancer proliferative disorders include neuro-fibromatosis, atherosclerosis, pulmonary fibrosis, arthritis, psoriasis, glomerulonephritis, restenosis, proliferative diabetic retinopathy (PDR), hypertrophic scar formation, inflammatory bowel disease, transplantation rejection, angiogenesis, and endotoxic shock.

In some embodiments, the compounds of the invention are used to treat cancers of the prostate, lung, colon, and breast.

Accordingly, the present invention further provides a method of treating a cancer selected from lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, pharyngeal cancer, laryngeal cancer, small intestinal cancer, non-Hodgkin lymphoma, and villous colon adenoma in a patient in need thereof, comprising administering to said patient a therapeutically effective amount of a compound of the invention or pharmaceutically acceptable salt thereof.

In a further aspect, the present invention provides methods of inhibiting the tumor growth in a patient in need thereof, comprising administering to said patient a therapeutically effective amount of a compound of the invention, or pharmaceutically acceptable salt thereof.

In some embodiments, the cancer is characterized by activity of PDK1. In some embodiments, the cancer is characterized by activity of a PDK1 variant such as PDK1^{T354M} or PDK1^{D527E}.

In further embodiments, the present invention provides a method for inhibition of Cdk1 and/or Cdk2. Another embodiment provides a methods of treating diseases such as cancer which are responsive to inhibition of Cdk1 and/or Cdk2 by administering a compound of the invention to a patient.

In further embodiments, the invention provides methods of inhibiting phosphorylation of Akt by administering a compound of the invention to a patient in need thereof. Another embodiment provides a method of treating diseases such as cancer which are responsive to inhibition of phosphorylation of Akt, by administering a compound of the invention to a patient. Another embodiment provides a method of inhibiting phosphorylation of Akt comprising contacting a cell with a compound of the invention.

In another aspect, the present invention provides a method of treating a disease is selected from neuro-fibromatosis, atherosclerosis, pulmonary fibrosis, arthritis, psoriasis, glomerulonephritis, restenosis, proliferative diabetic retinopathy, hypertrophic scar formation, inflammatory bowel disease, transplantation rejection, angiogenesis and endotoxic shock in a patient in need thereof, comprising administering to said patient a therapeutically effective amount of a compound of the invention, or pharmaceutically acceptable salt thereof.

In a further aspect, the present invention provides a kit for treating a cancer selected from lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, pharyngeal cancer, laryngeal cancer, small intestinal cancer, non-Hodgkin lymphoma, and villous colon adenoma in a patient in need thereof, comprising a compound according of the invention, or pharmaceutically acceptable salt thereof, and instructions comprising a direction to administer a therapeutically effective amount of said compound, or pharmaceutically acceptable salt thereof, to said patient.

In a further aspect, the present invention provides a kit for treating a disease selected from neuro-fibromatosis, atherosclerosis, pulmonary fibrosis, arthritis, psoriasis, glomerulonephritis, restenosis, proliferative diabetic retinopathy, hypertrophic scar formation, inflammatory bowel disease, transplantation rejection, angiogenesis and endotoxic shock in a patient in need thereof, comprising a compound of the invention, or pharmaceutically acceptable salt thereof, and instructions comprising a direction to administer a therapeutically effective amount of said compound, or pharmaceutically acceptable salt thereof, to said patient.

In a further aspect, the present invention provides a kit for inhibiting the tumor growth in a patient in need thereof, comprising a compound of the invention, or pharmaceutically acceptable salt thereof, and instructions comprising a direction to administer a therapeutically effective amount of said compound, or pharmaceutically acceptable salt thereof, to said patient.

In a further aspect, the present invention provides a kit for inhibiting PDK1 or a PDK1 variant in a patient in need thereof, comprising a compound of the invention, or pharmaceutically acceptable salt thereof, and instructions comprising a direction to administer an effective amount of said compound, or pharmaceutically acceptable salt thereof, to said patient.

The present invention further provides use of the compounds of the invention for the preparation of medicament for use in treating the cancers and diseases described herein as well as for inhibiting tumor and inhibiting PDK1 or a PDK1 variant in an individual.

The present invention further provides a compound of the invention for the for use in treating the cancers and diseases described herein as well as for inhibiting tumor and inhibiting PDK1 or a PDK1 variant in an individual.

The compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of present invention can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds of the invention.

The processes described herein can be monitored according to any suitable method known in the art. For example, product formation can be monitored by spectroscopic means, such as nuclear magnetic resonance spectroscopy (e.g., ¹H or ¹³C NMR) infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety. Adjustments to the protecting groups and formation and cleavage methods described herein may be adjusted as necessary in light of the various substituents.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

### EXAMPLES

The following examples, from Table 1, are intended to be illustrative and are not intended to limit the scope of the invention. Presented below are illustrative procedures for synthesizing compounds of the present invention listed in Table I. Compounds of the present invention can also be prepared by using procedures outlined in PCT/US2007/088392.

### Example 6

### (Table 1): Synthesis of (2-(2-(phenylamino)-7-(piperidin-4-yloxy) quinazolin-6-yl-) thiazol-4-yl)methanol

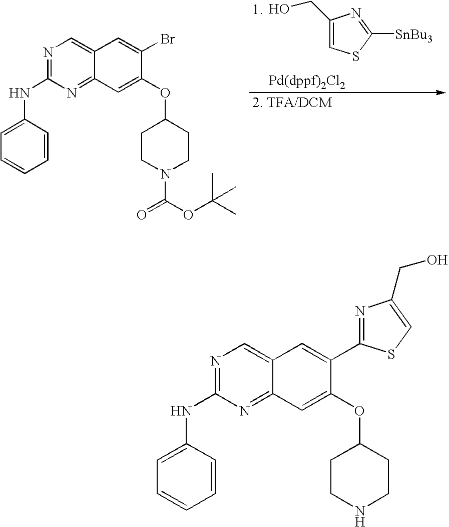

### Step 1. Preparation of tert-butyl-4-(6-(4-(hydroxymethyl)thiazol-2-yl)-2-(phenylamino) quinazolin-7-yloxy)piperidin-1-carboxylate

A mixture of tert-butyl-4-(6-bromo-2-(phenylamino) quinazolin-7-yloxy)piperidine-1-carboxylate (1 eq, see example 1020 for synthesis), 4-((tert-butyldimethylsilyloxy)methyl)-2-(tributylstannyl)thiazole (3 eq, Prepared as in example 252), TEA (3.5 eq) and Pd (dppf)₂Cl₂ (0.1 eq) in DMF (1.5 ml) was heated in microwave at 120° C. for 10 min. The reaction mixture was partitioned between ethylacetate and water. Ethylacetate layer was separated and washed with water, brine and dried over sodium sulfate. Filtered, evaporated and purified by semi-prep HPLC to provide pure product in 50% yield ES/MS m/z 534.2 (MH⁺).

### Step 2. Preparation of (2-(2-(phenylamino)-7-(piperidin-4-yloxy) quinazolin-6-yl-) thiazol-4-yl)methanol

For synthesis, see example 1020 (Table 3), step 6. ES/MS m/z 434.2

### Example 17

### (Table 1): methyl 2-(2-(3-fluorophenylamino)-7-(piperidin-4-yloxy) quinazolin-6-yl)thiazole-4-carboxylate

### The Subject Compound was Prepared According to the General Scheme Below:

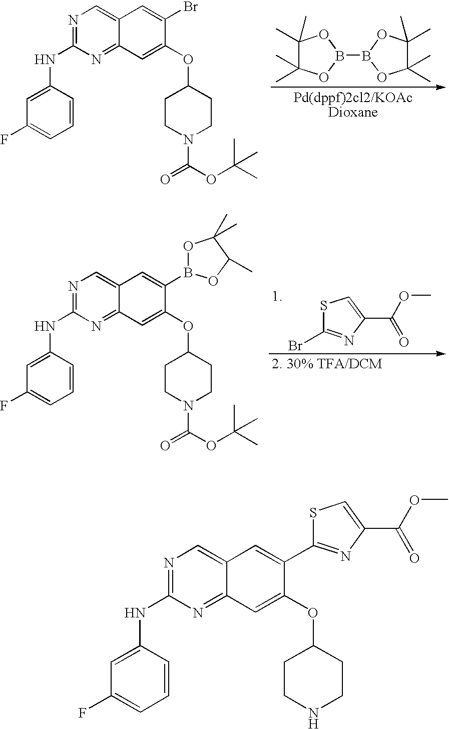

### Step 1. Preparation of tert-butyl 4-(2-(3-fluorophenylamino)-6-(4,4,5,5-tetramethyl-1,3,2-dioxaborolan-2-yl)quinazolin-7-yloxy)piperidine-1-carboxylate

To a mixture of tert-butyl 4-(6-bromo-2-(3-fluorophenylamino)quinazolin-7-yloxy)piperidine-1-carboxylate (for synthesis, see example 1020)(1 eq), KOAc (2 eq) and bis(pinacolato)diborane (1.6 eq) in dioxane (10 ml)nitrogen was purged for 10 min followed by the addition of Pd(dppf)2Cl2(0.02 eq). The reaction mixture was heated in sealed tube overnight at 80° C. The solvent was evaporated and crude was partitioned between ethyl acetate and water. The ethyl acetate layer was washed with brine, dried over sodium sulfate. Filtered, evaporated and dried. The residue was triturated with ether and solid was filtered, and dried to provide product as a brown solid in 55% yield. ES/MS m/z 565.2 (MH⁺)

### Step 2. Preparation of methyl 2-(2-(3-fluorophenylamino)-7-(piperidin-4-yloxy)quinazolin-6-yl)thiazole-4-carboxylate

For synthesis, see example 1020 (Table 3). ES/MS m/z 480.0 (MH⁺).

### Example 28

### (Table 1): Synthesis of N-(4-(1H-1,2,4-triazol-5-yl)phenyl)-7-(piperidin-4-yloxy)-6-(thiazol-2-yl)quinazolin-2-amine

The subject compound was prepared according to the general Scheme below:

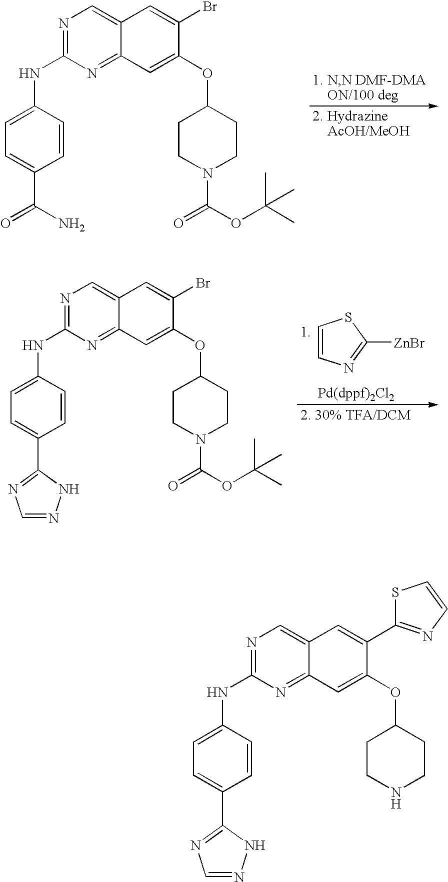

### Step 1 Preparation of tert-butyl-4-(2-(4-(1H-1,2,4-triazol-5-yl)phenylamino)-6-bromo quinazolin-7-yloxy)piperidine-1-carboxylate

(1) A solution of tert-butyl-4-(6-bromo-2-(4-carbamoylphenylamino) quinazolin-7-yloxy)piperidine-1-carboxylate (100 mg, see example 1020 for synthesis) in 2 ml of N,N dimethylformamide dimethylacetal was heated overnight at 100° C. The solvent was evaporated on rotovap and crude was proceed for next step. ES/MS m/z 597.1/599.1 (MH⁺).

(2) To above in methanol (2 ml) at 0° C. was added acetic acid (1.2 eq) and hydrazine (5 eq). The reaction mixture was warm to room temperature and stirred overnight. The product was purified by semi preparative HPLC and obtained as a yellow solid in 40% yield. ES/MS m/z 566.0/568.0 (MH⁺).

### Step 2 Preparation of tert-butyl-4-(2-(4-(1H-1,2,4-triazol-5-yl)phenylamino)-6-(thiazol-2-yl)quinazolin-7-yloxy)piperidine-1-carboxylate

For synthesis, see example 1020, step 5. ES/MS m/z 571.2 (MH⁺).

### Step 3 Preparation of N-(4-(1H-1,2,4-triazol-5-yl)phenyl)-7-(piperidin-4-yloxy)-6-(thiazol-2-yl)quinazolin-2-amine

For synthesis, see example 1020 (Table 3), step 6. ES/MS m/z 471.2 (MH⁺)

### Example 33

### (Table 1): Synthesis of N-(4-((1H-pyrazol-5-yl)methyl)phenyl)-7-(piperidin-4-yloxy)-6-(thiazol-2-yl)quinazolin-2-amine

The subject compound was prepared according to the general

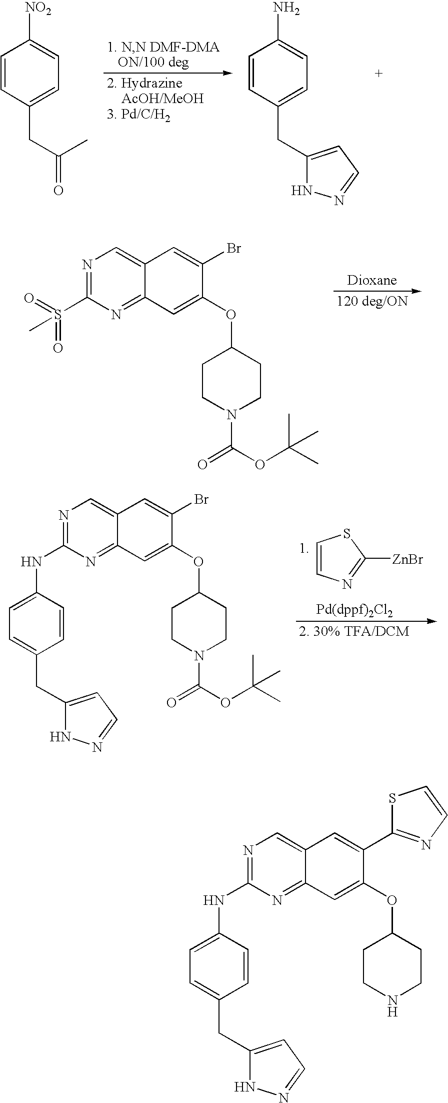

Scheme below: Step 1 Preparation of 5-(4-nitrobenzyl)-1H-pyrazole

For synthesis, see example 254, step 1 & 2. ES/MS m/z 204.1 (MH⁺)

Step 2 Preparation of 4-((1H-pyrazol-5-yl)methyl)aniline

To 5-(4-nitrobenzyl)-1H-pyrazole (500 mg) in methanol (5 ml) was added 100 mg of 10% Pd/C and stirred under H2 atmosphere for 3 h at room temperature. Filtered through celite, washed with methanol, evaporated and dried under vacuum. Yield 70%, ES/MS m/z 174.1 (MH⁺)

### Step 3 Preparation of N-(4-((1H-pyrazol-5-yl)methyl)phenyl)-7-(piperidin-4-yloxy)-6-(thiazol-2-yl)quinazolin-2-amine

For synthesis, see example 1020 (Table 3). ES/MS m/z 484.2 (MH⁺).

### Example 34

### (Table 1): Synthesis of 7-(piperidin-4-yloxy)-N-(3-(tetrahydro-2H-pyran-4-yl)phenyl-6-(thiazol-2-yl)quinazolin-2-amine

The subject compound was prepared according to the general Scheme below

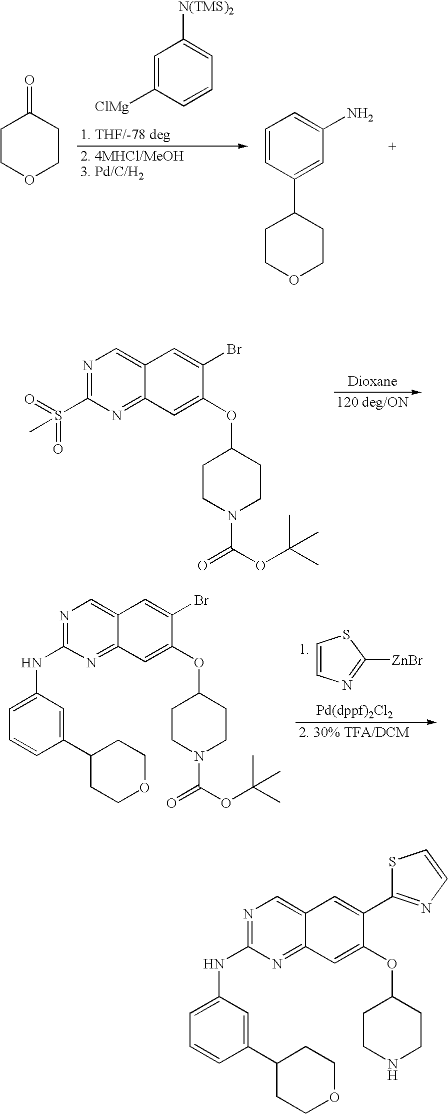

### Step 1 Preparation of 3-(tetrahydro-2H-pyran-4-yl)aniline

(1) To tetrahydro-4H-pyran-4-one (1 eq) in THF (2 ml) was added drop wise at −78° C. 3-[bis(TMS)amino-phenyl]magnesium chloride (1.2 eq). and stirred for 3 hr at this temperature. The reaction was quenched with ammonium chloride and compound was extracted in ethyl acetate. The organic layer was washed with brine, dried over sodium sulfate. Filtered, evaporated and dried to provide product as a yellow viscous liquid in quantitative yield. ES/MS m/z 194.1 (MH⁺)
(2) To 500 mg of above was added 4 ml of methanol and 4 ml of HCl (4M solution in dioxane) and heated in microwave at 120° C. for 10 min. The solvent was evaporated and crude was partitioned between ethyl acetate and water. The ethyl acetate layer was washed with brine, dried over sodium sulfate. Filtered, evaporated and dried to provide 3-(3,6-dihydro-2H-pyran-4-yl)aniline in quantitative yield. ES/MS m/z 176.2 (MH⁺)

(3) 3-(3,6-dihydro-2H-pyran-4-yl)aniline obtained in above step and 50 mg of 10% Pd/C in methanol (5-7 ml) was stirred under H2 atmosphere for 4 h at room temperature. Filtered through celite, washed with methanol, evaporated and dried under vacuum to provide 3-(tetrahydro-2H-pyran-4-yl)aniline as a brown solid. ES/MS m/z 174.1 (MH⁺)

### Step 2 Synthesis of 7-(piperidin-4-yloxy)-N-(3-(tetrahydro-2H-pyran-4-yl)phenyl-6-(thiazol-2-yl)quinazolin-2-amine

For synthesis, see example 1020 (Table 3). ES/MS m/z 488.2 (MH⁺).

### Example 62

### (Table 1)—Preparation of (S)-6-bromo-N-(3-(1-methyl-1H-pyrazol-4-yl)-5-(1-methylpyrrolidin-3-yloxy)phenyl)quinazolin-2-amine

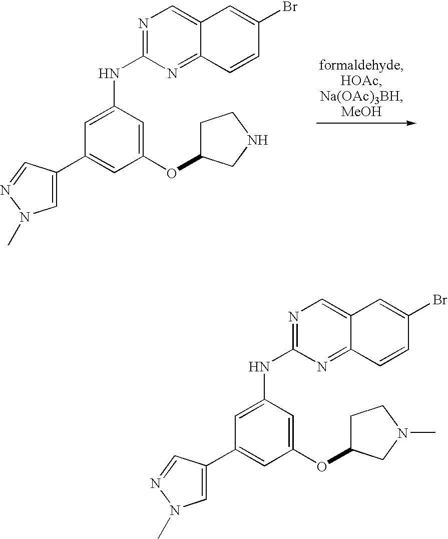

To a 0.15 M solution of (S)-6-bromo-N-(3-(1-methyl-1H-pyrazol-4-yl)-5-(pyrrolidin-3-yloxy)phenyl)quinazolin-2-amine (1.0 eq) in methanol was added a 37% solution of formaldehyde in water (5.0 eq) and glacial acetic acid (20 eq). The reaction was stirred for 45 min at ambient temperature. Sodium triacetoxyborohydride (3.0 eq) was added, and the reaction was stirred for an additional 30 min. The reaction was quenched by the addition of water and then concentrated. A 10:1 mixture of DCM:methanol was added. Aqueous sodium carbonate and sodium hydroxide were added until the pH of the aqueous phase was about 11. The layers were separated. The aqueous phase was extracted with additional DCM:methanol. The combined organic extracts were dried over sodium sulfate, filtered, and concentrated. The material could be purified further by reverse-phase HPLC and lyophilized to give an analytically pure sample of (S)-6-bromo-N-(3-(1-methyl-1H-pyrazol-4-yl)-5-(1-methylpyrrolidin-3-yloxy)phenyl)quinazolin-2-amine. ES/MS m/z 479/481 (MH⁺).

### Example 134

### (Table 1): 7-(1-(tert-butoxycarbonyl)piperidin-4-yloxy)-2-(3-fluorophenylamino)quinazoline-6-carboxylic acid

The subject compound was prepared according to the general Scheme below:

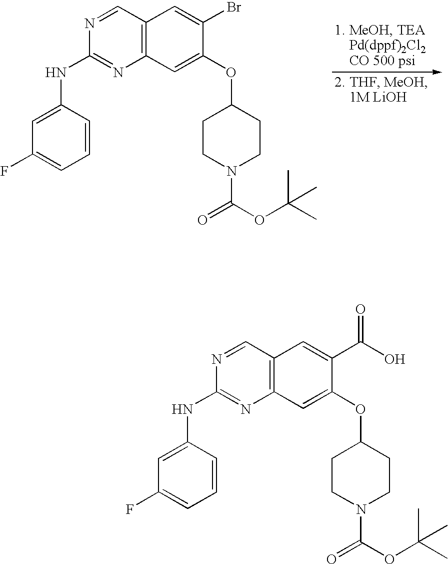

### Step 1: methyl 7-(1-(tert-butoxycarbonyl)piperidin-4-yloxy)-2-(3-fluorophenylamino)quinazoline-6-carboxylate

To the reaction mixture of, tert-butyl 4-(6-bromo-2-(3-fluorophenylamino)quinazolin-7-yloxy)piperidine-1-carboxylate (775 mg, 1.5 mmol) in 9 ml of MeOH in a steel bomb was added Pd(dppf)₂Cl₂(122 mg, 0.15 mmol) and TEA (0.630 ml, 4.5 mmol). The steel bomb was sealed and carefully CO was added to 500 psi. and slowly released (3× in a Hood) then recharged to 500 psi. and stirred at 90° C. for 20 hours. This reaction mixture was cooled and vented in a hood. The crude reaction mixture was concentrated. The crude residue was purified by silica gel column chromatography and concentrated in vaccuo to give, methyl 7-(1-(tert-butoxycarbonyl)piperidin-4-yloxy)-2-(3-fluorophenylamino)quinazoline-6-carboxylate (444 mg). ES/MS m/z 497.1 (MH⁺).

### Step 2: 7-(1-(tert-butoxycarbonyl)piperidin-4-yloxy)-2-(3-fluorophenylamino)quinazoline-6-carboxylic acid

To the reaction mixture of methyl 7-(1-(tert-butoxycarbonyl)piperidin-4-yloxy)-2-(3-fluorophenylamino)quinazoline-6-carboxylate (415 mg, 0.837 mmol) in 3.2 ml of THF and 1.8 ml of MeOH was added 1M LiOH (1.67 ml, 1.67 mmol). The crude reaction mixture was stirred at room temperature for 5 hours or until done by LCMS. The crude reaction was concentrated to solid, 15 ml of 1:1 ACN/Water solution was added and the pH was adjusted with 1N HCl carefully to a pH of (5-6). The crude solution was lyophilized and used with out further purification to give, as crude solid 7-(1-(tert-butoxycarbonyl)piperidin-4-yloxy)-2-(3-fluorophenylamino)quinazoline-6-carboxylic acid, (457 mg). ES/MS m/z 483.2 (MH⁺).

### Example 136

### (Table 1): 2-(3-fluorophenylamino)-7-(piperidin-4-yloxy)quinazoline-6-carbonitrile

The subject compound was prepared according to the general Scheme below:

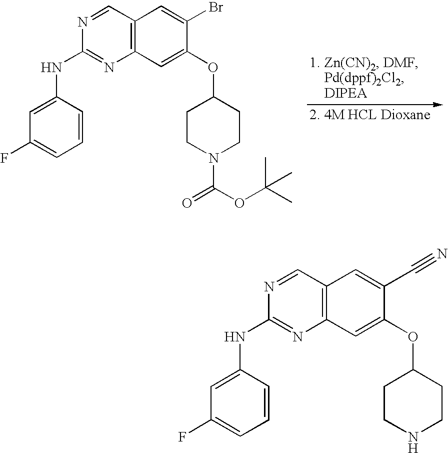

### Step 1: tert-butyl 4-(6-cyano-2-(3-fluorophenylamino)quinazolin-7-yloxy)piperidine-1-carboxylate

To the reaction mixture of, tert-butyl 4-(6-bromo-2-(3-fluorophenylamino)quinazolin-7-yloxy)piperidine-1-carboxylate (300 mg, 0.58 mmol) in 4.5 ml of DMF was added Pd(dppf)₂Cl₂ (72 mg, 0.087 mmol), Zn(CN)₂ (275 mg, 2.32 mmol) and DIPEA (0.303 ml, 1.74 mmol).). The reaction mixture was heated by microwave at 170° C. for 800 seconds. To the crude reaction mixture was add 150 ml ethyl acetate, washed with sat. sodium bicarbonate, water, salt solution, dried, purified by silica gel column chromatography and concentrated in vaccuo to give, tert-butyl 4-(6-cyano-2-(3-fluorophenylamino)quinazolin-7-yloxy)piperidine-1-carboxylate (135 mg). ES/MS m/z 464.3 (MH⁺).

### Step 2: 2-(3-fluorophenylamino)-7-(piperidin-4-yloxy)quinazoline-6-carbonitrile

To the solid tert-butyl 4-(6-cyano-2-(3-fluorophenylamino)quinazolin-7-yloxy)piperidine-1-carboxylate (20 mg, 0.043 mmol) was added 4M HCl in dioxane (2.0 ml, 4.0 mmol). The reaction mixture was stirred at room temperature for 1 hour. The crude reaction mixture was concentrate, about 1 ml of DMF was added, filtered, purified on prep HPLC and lyophilized to give, 2-(3-fluorophenylamino)-7-(piperidin-4-yloxy)quinazoline-6-carbonitrile, as TFA salt (5.8 mg). ES/MS m/z 364.2 (MH+)

### Example 137

### (Table 1): N-(3-fluorophenyl)-7-(piperidin-4-yloxy)-6-(1H-tetrazol-5-yl)quinazolin-2-amine

The subject compound was prepared according to the general Scheme below:

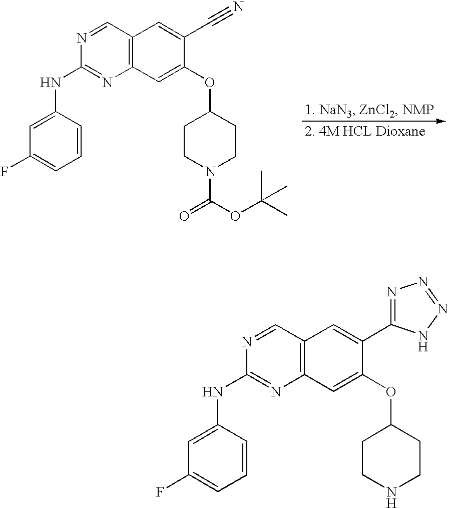

### Step 1: tert-butyl 4-(2-(3-fluorophenylamino)-6-(1H-tetrazol-5-yl)quinazolin-7-yloxy)piperidine-1-carboxylate

To the reaction mixture of, tert-butyl 4-(6-cyano-2-(3-fluorophenylamino)quinazolin-7-yloxy)piperidine-1-carboxylate (50 mg, 0.101 mmol) in 1.0 ml of NMP was added ZnCl₂ (68 mg, 0.505 mmol) and NaN₃ (65 mg, 1.0 mmol). The reaction was heated by microwave at 170° C. for 800 seconds. The crude material was purified on prep HPLC and free based and concentrated to solid to give, tert-butyl 4-(2-(3-fluorophenylamino)-6-(1H-tetrazol-5-yl)quinazolin-7-yloxy)piperidine-1-carboxylate (14 mg). ES/MS m/z 507.3 (MH⁺).

### Step 2: N-(3-fluorophenyl)-7-(piperidin-4-yloxy)-6-(1H-tetrazol-5-yl)quinazolin-2-amine

To the solid tert-butyl 4-(2-(3-fluorophenylamino)-6-(1H-tetrazol-5-yl)quinazolin-7-yloxy)piperidine-1-carboxylate (14 mg, 0.0275 mmol) was added 4M HCl in dioxane (2.0 ml, 4.0 mmol). The reaction mixture was stirred at room temperature for 1 hour. The crude reaction mixture was concentrate, about 1 ml of DMF was added, filtered, purified on prep HPLC and lyophilized to give, N-(3-fluorophenyl)-7-(piperidin-4-yloxy)-6-(1H-tetrazol-5-yl)quinazolin-2-amine as TFA salt (6.3 mg). ES/MS m/z 407.2 (MH+).

### Example 138

### (Table 1): N-(3-fluorophenyl)-6-(4-methyl-1H-imidazol-2-yl)-7-(piperidin-4-yloxy)quinazolin-2-amine

The subject compound was prepared according to the general Scheme below:

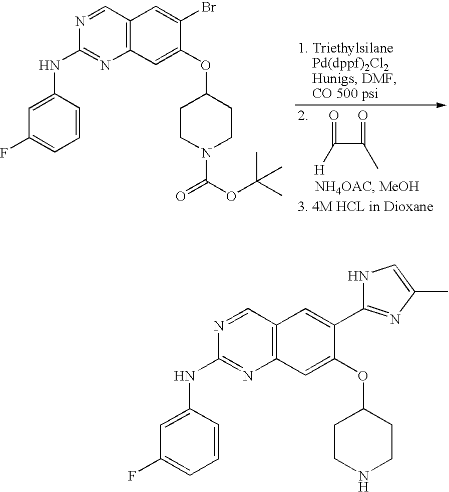

### Step 1: tert-butyl 4-(2-(3-fluorophenylamino)-6-formylquinazolin-7-yloxy)piperidine-1-carboxylate

To the reaction mixture of, tert-butyl 4-(6-bromo-2-(3-fluorophenylamino)quinazolin-7-yloxy)piperidine-1-carboxylate (300 mg, 0.58 mmol) in 2.3 ml of DMF in a steel bomb was added Pd(dppf)₂Cl₂ (47 mg, 0.058 mmol), triethylsilane (202 mg, 1.74 mmol) and TEA (0.203 ml, 1.45 mmol). The steel bomb was sealed and carefully CO was added to 500 psi. and slowly released (3× in a Hood) then recharged to 500 psi. and stirred at 95-100° C. for 20 hours. This reaction mixture was cooled and vented in a hood. The crude reaction mixture was filtered, purified on prep HPLC and lyophilized to give, tert-butyl 4-(2-(3-fluorophenylamino)-6-formylquinazolin-7-yloxy)piperidine-1-carboxylate as TFA salt (130 mg). ES/MS m/z 467.2 (MH⁺).

### Step 2: tert-butyl 4-(2-(3-fluorophenylamino)-6-(4-methyl-1H-imidazol-2-yl)quinazolin-7-yloxy)piperidine-1-carboxylate

To the reaction mixture of tert-butyl 4-(2-(3-fluorophenylamino)-6-formylquinazolin-7-yloxy)piperidine-1-carboxylate (25 mg, 0.54 mmol) in 0.9 ml of MeOH was added ammonium acetate (43 mg, 0.54 mmol) and a 40% solution of 2-oxopropanal in water (0.049 ml, 0.27 mmol). The reaction mixture was heated at 65-70° C. for 90 minutes or until done by LCMS. To the crude reaction mixture was concentrate to solid to give a crude product, tert-butyl 4-(2-(3-fluorophenylamino)-6-(4-methyl-1H-imidazol-2-yl)quinazolin-7-yloxy)piperidine-1-carboxylate used in next step without purification. ES/MS m/z 519.3 (MH⁺).

### Step 3: N-(3-fluorophenyl)-6-(4-methyl-1H-imidazol-2-yl)-7-(piperidin-4-yloxy)quinazolin-2-amine

To the crude reaction mixture of tert-butyl 4-(2-(3-fluorophenylamino)-6-(4-methyl-1H-imidazol-2-yl)quinazolin-7-yloxy)piperidine-1-carboxylate (0.054 mmol) was added 4M HCl in Dioxane (4.0 ml, 16.0 mmol). The reaction mixture was stirred at room temperature for 1 hours or until done by LCMS. The crude reaction mixture was concentrate, about 1 ml of DMF was added, filtered, purified on prep HPLC and lyophilized to give, N-(3-fluorophenyl)-6-(4-methyl-1H-imidazol-2-yl)-7-(piperidin-4-yloxy)quinazolin-2-amine as TFA salt (6.5 mg). ES/MS m/z 419.2 (MH⁺).

### Example 140

### (Table 1): N-(3-fluorophenyl)-7-(piperidin-4-yloxy)-6-(pyridin-2-ylethynyl)quinazolin-2-amine

The subject compound was prepared according to the general Scheme below:

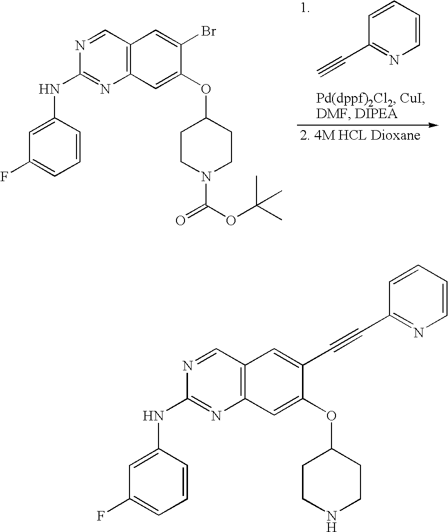

### Step 1: tent-butyl 4-(2-(3-fluorophenylamino)-6-(pyridin-2-ylethynyl)quinazolin-7-yloxy)piperidine-1-carboxylate

To the reaction mixture of, tert-butyl 4-(6-bromo-2-(3-fluorophenylamino)quinazolin-7-yloxy)piperidine-1-carboxylate (90 mg, 0.174 mmol) in 1.0 ml of DMF was added Pd(dppf)₂Cl₂(14.3 mg, 0.0174 mmol), CuI (7.3 mg, 0.038 mmol), 2-ethynylpyridine (44 mg, 0.435 mmol) and last add DIPEA (0.091 ml, 0.522 mmol). The reaction mixture was heated at 95° C. for 75 minutes or until done by LCMS. The crude reaction mixture was concentrate, about 1 ml of DMF was added, filtered, purified on prep HPLC and lyophilized to give, tert-butyl 4-(2-(3-fluorophenylamino)-6-(pyridin-2-ylethynyl)quinazolin-7-yloxy)piperidine-1-carboxylate as TFA salt (48 mg). ES/MS m/z 540.3 (MH+)

### Step 2: N-(3-fluorophenyl)-7-(piperidin-4-yloxy)-6-(pyridin-2-ylethynyl)quinazolin-2-amine

To the solid tert-butyl 4-(2-(3-fluorophenylamino)-6-(pyridin-2-ylethynyl)quinazolin-7-yloxy)piperidine-1-carboxylate (24 mg, 0.0445 mmol) was added 4M HCl in dioxane (2.0 ml, 4.0 mmol). The reaction mixture was stirred at room temperature for 1 hour. The crude reaction mixture was concentrate, about 1 ml of DMF was added, filtered, purified on prep HPLC and lyophilized to give, N-(3-fluorophenyl)-7-(piperidin-4-yloxy)-6-(pyridin-2-ylethynyl)quinazolin-2-amine, as TFA salt (9.8 mg). ES/MS m/z 440.1 (MH+)

### Example 142

### (Table 1): N-(3-fluorophenyl)-7-(piperidin-4-yloxy)-6-(2-(pyridin-2-yl)ethyl)quinazolin-2-amine

The subject compound was prepared according to the general Scheme below:

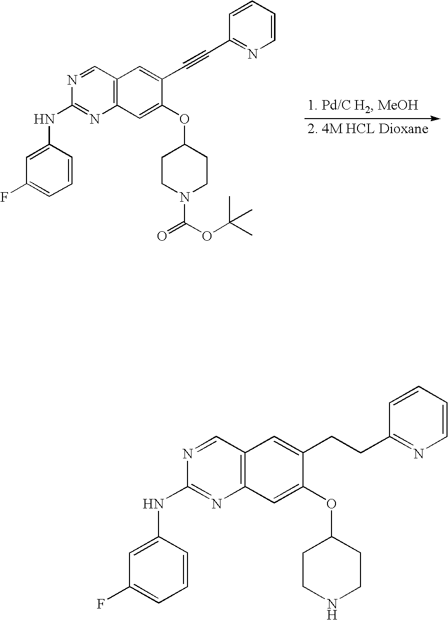

### Step 1: tert-butyl 4-(2-(3-fluorophenylamino)-6-(2-(pyridin-2-yl)ethyl)quinazolin-7-yloxy)piperidine-1-carboxylate

To the starting solid, tert-butyl 4-(2-(3-fluorophenylamino)-6-(pyridin-2-ylethynyl)quinazolin-7-yloxy)piperidine-1-carboxylate (25 mg, 0.046 mmol) was added 10% Pd on Carbon (25 mg, 100% by wt.) under argon. Under argon with a syringe carefully add 3.0 ml methanol. To this reaction mixture was added a hydrogen balloon and was evacuated and refilled 5 times. The reaction was stirred at room temperature for 20 hours or until done by LCMS. To the reaction mixture add ethyl acetate and under argon filtered through celite and washed with a 1:1 solution of ethyl acetate and methanol. The filtrate was concentrated in vaccuo to give, tert-butyl 4-(2-(3-fluorophenylamino)-6-(2-(pyridin-2-yl)ethyl)quinazolin-7-yloxy)piperidine-1-carboxylate as crude material used in next step. (25 mg). ES/MS m/z 544.2 (MH⁺).

### Step 2: N-(3-fluorophenyl)-7-(piperidin-4-yloxy)-6-(2-(pyridin-2-yl)ethyl)quinazolin-2-amine

To the crude solid, tert-butyl 4-(2-(3-fluorophenylamino)-6-(2-(pyridin-2-yl)ethyl)quinazolin-7-yloxy)piperidine-1-carboxylate (25 mg, 0.046 mmol) was added 4M HCl in dioxane (2.0 ml, 4.0 mmol). The reaction mixture was stirred at room temperature for 1 hour. The crude reaction mixture was concentrate, about 1 ml of DMF was added, filtered, purified on prep HPLC and lyophilized to give, N-(3-fluorophenyl)-7-(piperidin-4-yloxy)-6-(2-(pyridin-2-yl)ethyl)quinazolin-2-amine as TFA salt (4.8 mg). ES/MS m/z 444.0 (MH+).

### Example 149

### (Table 1)—Preparation of (S)-6-ethynyl-N-(3-(1-methyl-1H-pyrazol-4-yl)-5-(1-methylpyrrolidin-3-yloxy)phenyl)quinazolin-2-amine

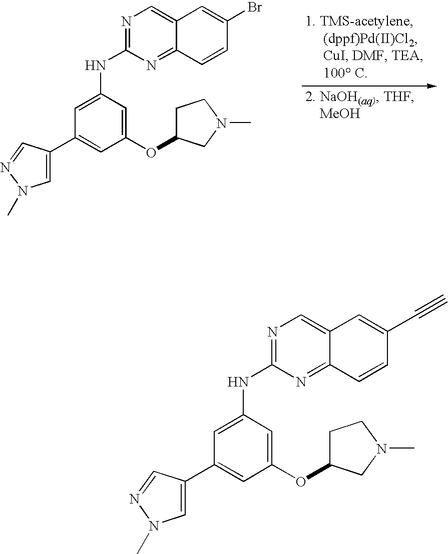

Step 1. Sonogashira

To a degassed 0.15 M solution of (S)-6-bromo-N-(3-(1-methyl-1H-pyrazol-4-yl)-5-(1-methylpyrrolidin-3-yloxy)phenyl)quinazolin-2-amine in 1:1 DMF:TEA was added trimethylsilylacetylene (TMS-acetylene) (4.0 eq); copper(I) iodide (0.10 eq); and [1,1′-bis(diphenylphosphino)ferrocene]dichloropalladium(II) complex with DCM (0.050 eq). The reaction was stirred at 100° C. for 1 h. The mixture was concentrated and used without further purification.

Step 2. Desilylation

To a 0.10 M solution of the product of Step 1 in 2:1 THF:MeOH was added 6.0 M aqueous sodium hydroxide (3.0 eq). The reaction was stirred for 20 min at ambient temperature. Volatiles were removed under reduced pressure, and the crude residue was purified by reverse-phase HPLC, and lyophilized to give (S)-6-ethynyl-N-(3-(1-methyl-1-pyrazol-4-yl)-5-(1-methylpyrrolidin-3-yloxy)phenyl)quinazolin-2-amine as its trifluoroacetic acid salt. ES/MS m/z 425.1 (MH⁺).

### Example 152

### (Table 1)—Preparation of (S)-6-bromo-N-(3-(1-methyl-1H-pyrazol-4-yl)-5-(pyrrolidin-3-yloxy)phenyl)quinazolin-2-amine

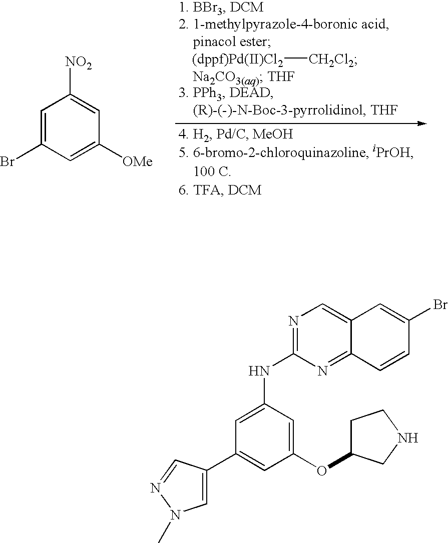

Step 1. Demethylation

To a 0.70 M solution of 1-bromo-3-methoxy-5-nitrobenzene (1.0 eq) in DCM was added a 1.0 M solution of boron tribromide in DCM (2.2 eq) while cooling in an ice/water bath. The reaction was stirred for 4 d at ambient temperature. The mixture was cooled to −78 C and quenched with methanol (15 eq). The mixture was returned to ambient temperature. Volatiles were removed under reduced pressure. The crude material was triturated with 5:1 water:methanol (210 mL), filtered, and rinsed with additional solvent to give 31.9 g of the desired product as a lavender-grey solid after drying in a desiccator.

Step 2. Suzuki

The product from step 1 (1.0 eq) was dissolved in THF to make a 0.30 M solution. A 2.0 M aqueous solution of sodium carbonate (3.5 eq) was added. The mixture was degassed by bubbling argon through the solution for 10 min. 1-Methylpyrazole-4-boronic acid, pinacol ester (1.2 eq) and (dppf)Pd(II) Cl₂-CH₂Cl₂ (0.060 eq) were added. The mixture was stirred at 80 C for 90 min and then returned to ambient temperature. The mixture was diluted with ethyl acetate. Dilute aqueous hydrochloric acid was added until the pH of the aqueous layer was 5-6. The layers were separated. The aqueous phase was extracted with additional ethyl acetate. The combined organic extracts were dried over sodium sulfate, filtered, and concentrated. The crude product was purified by flash chromatography (ethyl acetate then 92:8 ethyl acetate:methanol) to give the desired product as a yellow-brown solid. ES/MS m/z 220.0 (MH⁺).

Step 3. Mitsunobu

To a 0.35M solution of triphenylphosphine (1.4 eq) in THF was added diethylazodicarboxylate (1.4 eq). The mixture was stirred 10 min at ambient temperature. (R)-(−)-N-Boc-3-pyrrolidinol (1.8 eq) was added. The mixture was stirred 20 min at ambient temperature. The product from step 2 (1.0 eq) was added. The mixture was stirred an additional 15 h. The crude mixture was concentrated and purified by flash chromatography (1:2 hexanes:EtOAc) to give the desired product as a thick oil. ES/MS m/z 388.9 (MH⁺).

Step 4. Reduction

A 0.10 M solution of the product from step 3 in methanol was purged with argon. Catalytic 10% palladium on carbon was added. The reaction vessel was purged and filled with hydrogen gas. The reaction was stirred at ambient temperature and pressure for 17 h. The mixture was filtered through Celite, and the filtrate was concentrated. The crude product was purified by flash chromatography (1:2 hexanes:EtOAc with 1% TEA to 96:4:1 ethyl acetate:methanol:TEA) to give the desired product as a white solid. ES/MS m/z 359.0 (MH⁺).

Step 5. Displacement

A 0.25 M suspension of the product from step 4 (1.0 eq) and 6-bromo-2-chloroquinazoline (1.0 eq) in 2-propanol was stirred at 100 C for 16 h. The mixture was concentrated and used without further purification. ES/MS m/z 565/567 (MH⁺).

Step 6. Deprotection

A 0.2 M solution of the product from step 5 in 1:1 DCM:TFA was stirred for 15 min. The mixture was concentrated. A 10:1 mixture of DCM:methanol was added. Aqueous sodium carbonate and sodium hydroxide were added until the pH of the aqueous phase was about 11. The layers were separated. The aqueous phase was extracted with additional DCM:methanol. The combined organic extracts were dried over sodium sulfate, filtered, and concentrated. The material could be purified further by reverse-phase HPLC and lyophilized to give an analytically pure sample of (S)-6-bromo-N-(3-(1-methyl-1H-pyrazol-4-yl)-5-(pyrrolidin-3-yloxy)phenyl)quinazolin-2-amine. ES/MS m/z 465/467 (MH⁺).

### Example 171

### (Table 1)—Preparation of 4-(3-(6-ethynylquinazolin-2-ylamino)-5-(1-methyl-1H-pyrazol-4-yl)benzyl)morpholine 4-oxide

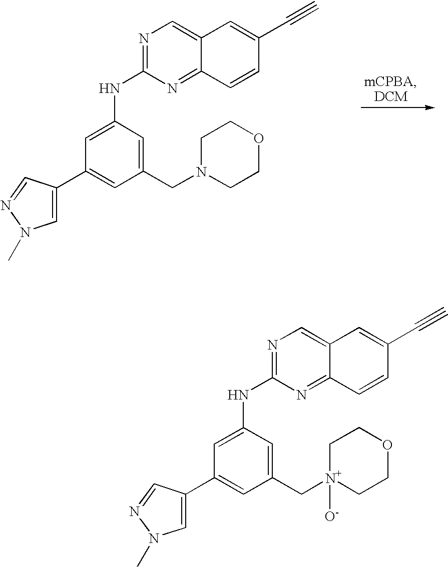

To a 0.15 M solution of 6-ethynyl-N-(3-(1-methyl-1H-pyrazol-4-yl)-5-(morpholinomethyl)phenyl)quinazolin-2-amine in DCM was added 3-chloroperoxybenzoic acid (1.2 eq). The reaction was stirred for 1 h at ambient temperature. The mixture was diluted with DCM, and a 0.5 M aqueous solution of sodium carbonate was added. The layers were separated. The organic phase was dried over sodium sulfate, filtered, and concentrated. The crude material was purified by reverse-phase HPLC and lyophilized to give 4-(3-(6-ethynylquinazolin-2-ylamino)-5-(1-methyl-1H-pyrazol-4-yl)benzyl)morpholine 4-oxide. ES/MS m/z 441.1 (MH⁺).

### Example 184

### (Table 1)—Preparation of (S)-2-(dimethylamino)-1-(3-(3-(6-ethynylquinazolin-2-ylamino)-5-(1-methyl-1H-pyrazol-4-yl)phenoxy)pyrrolidin-1-yl)ethanone

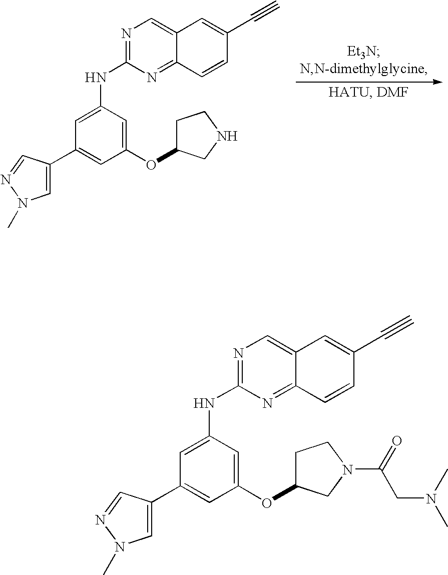

To a 0.15 M solution of (S)-6-ethynyl-N-(3-(1-methyl-1H-pyrazol-4-yl)-5-(pyrrolidin-3-yloxy)phenyl)quinazolin-2-amine (1.0 eq) in DMF was added triethylamine (3.0 eq), N,N-dimethylglycine (1.0 eq), and HATU (1.0 eq). The reaction was stirred for 20 min at ambient temperature. The crude reaction mixture was purified by reverse-phase HPLC and lyophilized to give (S)-2-(dimethylamino)-1-(3-(3-(6-ethynylquinazolin-2-ylamino)-5-(1-methyl-1H-pyrazol-4-yl)phenoxy)pyrrolidin-1-yl)ethanone. ES/MS m/z 496.1 (MH⁺).

### Example 188

### (Table 1)—Preparation of (S)-2-(dimethylamino)-1-(3-(3-(6-ethynylquinazolin-2-ylamino)-5-(1-methyl-1H-pyrazol-4-yl)phenoxy)pyrrolidin-1-yl)ethanone

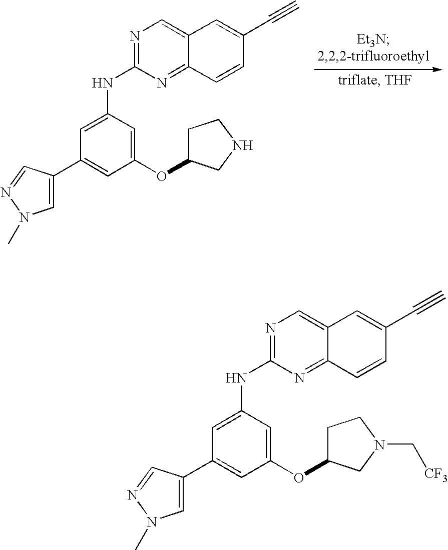

To a 0.15 M solution of (S)-6-ethynyl-N-(3-(1-methyl-1H-pyrazol-4-yl)-5-(pyrrolidin-3-yloxy)phenyl)quinazolin-2-amine (1.0 eq) in THF was added triethylamine (3.0 eq) and 2,2,2-trifluoroethyl triflate (1.5 eq). The reaction was stirred for 90 min at ambient temperature. The crude reaction mixture was concentrated and then purified by reverse-phase HPLC and lyophilized to give (S)-6-ethynyl-N-(3-(1-methyl-1H-pyrazol-4-yl)-5-(1-(2,2,2-trifluoroethyl)pyrrolidin-3-yloxy)phenyl)quinazolin-2-amine. ES/MS m/z 493.1 (MH⁺).

### Example 190

### (Table 1)—Preparation of 6-bromo-N-(3-(1-methyl-1H-pyrazol-4-yl)-5-(1-morpholinoethyl)phenyl)quinazolin-2-amine

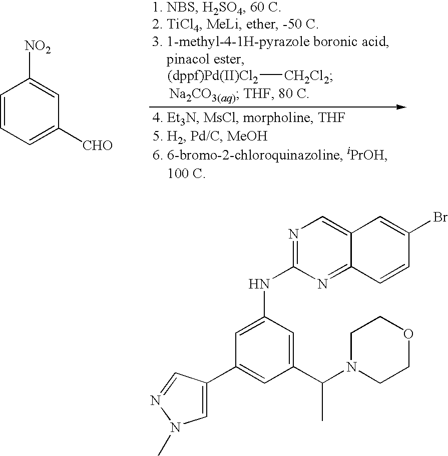

Step 1. Bromination

A 2.2 M mixture of 3-nitrobenzaldehyde (1.0 eq) in sulfuric acid was warmed to 60 C. N-Bromosuccinimide (1.2 eq) was added in three portions, 15 minutes apart. The solution was stirred for an additional 30 min and then returned to ambient temperature. The mixture was poured over crushed ice. The resulting precipitate was filtered off and rinsed with water until the pH of the filtrate was 4-5. The filter cake was additionally rinsed with hexanes and then dried in a desiccator to give the desired product as an off-white solid. GC/MS 229,231.

Step 2. Organotitanium Addition

Titanium tetrachloride (1.0 eq) was added to anhydrous ether at −78 C. A 1.6 M solution of methyllithium in ether (1.0 eq) was added over 10 min. The mixture was stirred for 1 h, allowing to warm to −50 C. A 0.1 M solution of the product from step 1 in ether (1.0 eq) was added over 20 min. The reaction was stirred for an additional 90 min and then poured into water. The product was extracted with ether. The combined organic extracts were washed with brine and then dried over sodium sulfate, filtered, and concentrated to give the desired product. GC/MS 245,247.

Step 3. Suzuki

The product from step 2 (1.0 eq) was dissolved in THF to make a 0.30 M solution. A 2.0 M aqueous solution of sodium carbonate (3.5 eq) was added. The mixture was degassed by bubbling argon through the solution for 5 min. 1-Methylpyrazole-4-boronic acid, pinacol ester (1.2 eq) and (dppf)Pd(II) Cl₂—CH₂Cl₂ (0.060 eq) were added. The mixture was stirred at 80 C for 90 min and then returned to ambient temperature. The mixture was diluted with ethyl acetate and saturated aqueous sodium bicarbonate. The layers were separated. The aqueous phase was extracted with additional ethyl acetate. The combined organic extracts were dried over sodium sulfate, filtered, and concentrated. The crude product was purified by flash chromatography (ethyl acetate then 97:3 ethyl acetate:methanol) to give the desired product. ES/MS m/z 248.0 (MH⁺).

Step 4. Displacement

A 0.20 M solution of the product from step 3 in THF was cooled to 0 C. Triethylamine (2.0 eq) was added followed by methanesulfonyl chloride (1.1 eq) in a dropwise fashion. The mixture was stirred for 3 h while warming to ambient temperature. Morpholine (2.0 eq) was added, and the reaction was stirred at 60 C for 15 h. Additional morpholine (1.0 eq) was added, and the reaction was stirred at 50 C for 3 days. Volatiles were removed under reduced pressure. Ethyl acetate and water were added. The layers were separated. The organic phase was washed with additional water and then dried over sodium sulfate, filtered, and concentrated to give the desired product. ES/MS m/z 317.0 (MH⁺)

Step 5. Reduction

A 0.050 M solution of the product from step 4 in methanol was purged with argon. Catalytic 10% palladium on carbon (20% by mass) was added. The reaction vessel was purged and filled with hydrogen gas. The reaction was stirred at ambient temperature and pressure for 2 days. The mixture was filtered through Celite, and the filtrate was concentrated to give the desired product as a crystalline solid. ES/MS m/z 287.0 (MH⁺).

Step 6. Displacement

To a 0.20 M suspension of the product from step 4 (1.0 eq) and 6-bromo-2-chloroquinazoline (1.0 eq) in 2-propanol was added HCl (4.0 M in dioxane, 1.0 eq). The mixture was stirred at 100 C for 16 h. The mixture was concentrated and could be used without further purification. Alternatively, the crude product could be purified by reverse-phase HPLC and lyophilized to give 6-bromo-N-(3-(1-methyl-1H-pyrazol-4-yl)-5-(1-morpholinoethyl)phenyl)quinazolin-2-amine as its trifluoracetic acid salt. ES/MS m/z 493/495 (MH⁺).

### Example 192

### (Table 1)—Preparation of (S)-2-(dimethylamino)-1-(3-(3-(6-ethynylquinazolin-2-ylamino)-5-(1-methyl-1H-pyrazol-4-yl)phenoxy)pyrrolidin-1-yl)ethanone

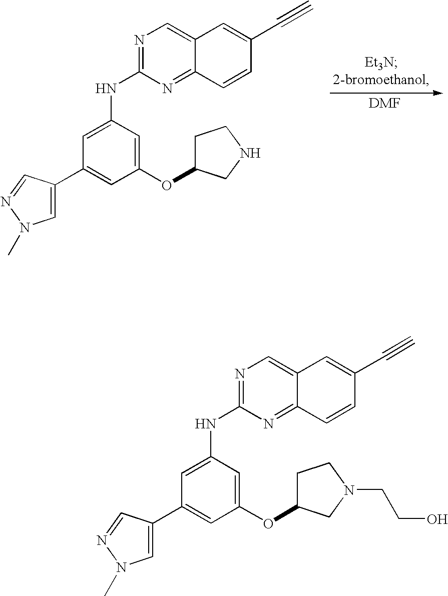

To a 0.3 M solution of (S)-6-ethynyl-N-(3-(1-methyl-1H-pyrazol-4-yl)-5-(pyrrolidin-3-yloxy)phenyl)quinazolin-2-amine (1.0 eq) in DMF was added triethylamine (3.0 eq) and 2-bromoethanol (1.5 eq). The reaction was stirred for 1 h at ambient temperature and 40 C for 2 h. The crude reaction mixture was concentrated and then purified by reverse-phase HPLC and lyophilized to give (S)-2-(3-(3-(6-ethynylquinazolin-2-ylamino)-5-(1-methyl-1H-pyrazol-4-yl)phenoxy)pyrrolidin-1-yl)ethanol. ES/MS m/z 455.1 (MH^{÷}).

### Example 202

### Preparation of 1-(3-(6-bromoquinazolin-2-ylamino)-5-(1-methyl-1H-pyrazol-4-yl)benzyl)pyrrolidin-3-ol

To a solution of 4-(4,4,5,5-tetramethyl-1,3,2-dioxaborolan-2-yl)-1H-pyrazole in THF was added sodium hydride (1.5 eq) and then added ethyl bromide (1.2 eq). Formation of the product was confirmed by LC/MS. It was quenched with a few drops of water and used. ES/MS m/z 223 (MH⁺).

### Example 213

### (Table 1)

### Preparation of N-(3-((2,6-dimethylmorpholino)methyl)-5-(1-methyl-1H-pyrazol-4-yl)phenyl)-6-ethynylquinazolin-2-amine

The subject compound was prepared according to the general Scheme below:

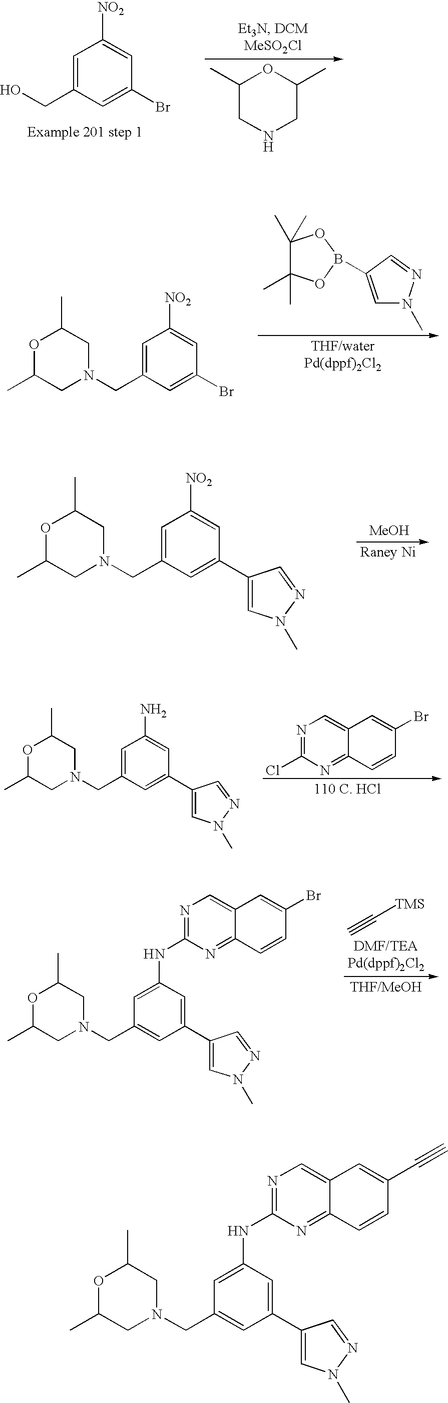

### 1. Preparation of 4-(3-bromo-5-nitrobenzyl)-2,6-dimethylmorpholine

To a solution (3-bromo-5-nitrophenyl)methanol in dichloromethane at 0° C. was added triethylamine (3 eq) and methanesulfonylchloride (1.5 eq) and the mixture was brought to ambient temperature. To the reaction was added 2,6-dimethylmorpholine (2 eq) and the mixture was stirred at ambient temperature for 16 h. Conversion to product was observed by LC/MS. The mixture was washed with brine and the organic layer was dried and concentrated to give 4-(3-bromo-5-nitrobenzyl)-2,6-dimethylmorpholine. ES/MS m/z 328 (MH⁺).

### 2. Preparation of 2,6-dimethyl-4-(3-(1-methyl-1H-pyrazol-4-yl)-5-nitrobenzyl)morpholine

To a solution of 2,6-dimethyl-4-(3-(1-methyl-1H-pyrazol-4-yl)-5-nitrobenzyl)morpholine in dimethoxyethane and 2M sodium carbonate was added 1-methyl-4-(4,4,5,5-tetramethyl-1,3,2-dioxaborolan-2-yl)-1H-pyrazol (3 eq) and the mixture was microwaved at 120° C. for 10 mins. Complete conversion to the product was observed by LC/MS. It was partitioned between ethylacetate and water and the organic layer was washed with brine and dried with sodium sulfate and concentrated. It was purified on silica gel to give 2,6-dimethyl-4-(3-(1-methyl-1H-pyrazol-4-yl)-5-nitrobenzyl)morpholine. ES/MS m/z 330 (MH⁺).

### 3. Preparation of 3-((2,6-dimethylmorpholino)methyl)-5-(1-methyl-1H-pyrazol-4-yl)aniline

To a solution 2,6-dimethyl-4-(3-(1-methyl-1H-pyrazol-4-yl)-5-nitrobenzyl)morpholine in methanol was added Raney Ni under nitrogen and the mixture was hydrogenated for 5 h and observed the formation of 3-((2,6-dimethylmorpholino)methyl)-5-(1-methyl-1H-pyrazol-4-yl)aniline. The catalyst was filtered off to give the product. ES/MS m/z 300 (MH⁺).

### 4. Preparation of 6-bromo-N-(3-((2,6-dimethylmorpholino)methyl)-5-(1-methyl-1H-pyrazol-4-yl)phenyl)quinazolin-2-amine

To a solution of (3-((2,6-dimethylmorpholino)methyl)-5-(1-methyl-1H-pyrazol-4-yl)aniline (1.1 eq) and few drops of 4N HCl in dioxane in isopropanol was added 2-chloro-6-bromoquinazoline (1 eq) and the mixture was heated to 110° C. Complete conversion to the product was observed by LC/MS. The product was filtered and washed with isopropanol to give 6-bromo-N-(3-((2,6-dimethylmorpholino)methyl)-5-(1-methyl-1H-pyrazol-4-yl)phenyl)quinazolin-2-amine. ES/MS m/z 506 (MH⁺).

### 5. Preparation of N-(3-((2,6-dimethylmorpholino)methyl)-5-(1-methyl-1H-pyrazol-4-yl)phenyl)-6-ethynylquinazolin-2-amine

To a solution of 6-bromo-N-(3-((2,6-dimethylmorpholino)methyl)-5-(1-methyl-1H-pyrazol-4-yl)phenyl)quinazolin-2-amine (1 eq) in 1:1 DMF and triethylamine was added TMS acetylene (4 eq) and CuI (0.2 eq) and Pd(dppf)₂Cl₂ (0.2 eq). The mixture was microwaved at 120° C. for 20 mins. Complete conversion to the product was observed by LC/MS. The mixture was partitioned between ethyl acetate and water. The organic lat=yer was washed with brine and dries with sodium sulfate to give the TMS protected N-(3-((2,6-dimethylmorpholino)methyl)-5-(1-methyl-1H-pyrazol-4-yl)phenyl)-6-ethynylquinazolin-2-amine. To it was added 1:1 THF/MeOH and 6N sodium hydroxide (3 eq). Instantaneous deprotection of the TMS group was seen on LC/MS. The mixture was cooled with ice/water bathe and 1N HCl was added to neutralize the reaction and the mixture was partitioned between ethyl acetate and water. The organic layer was separated and concentrated and purified on prep HPLC to give N-(3-(2,6-dimethylmorpholino)methyl)-5-(1-methyl-1H-pyrazol-4-yl)phenyl)-6-ethynylquinazolin-2-amine. ES/MS m/z 453.2 (MH⁺).

### Example 242

### (Table 1)

### Preparation of 4-(5-chloroquinazolin-2-ylamino)benzenesulfonamide

The subject compound was prepared according to the general Scheme below:

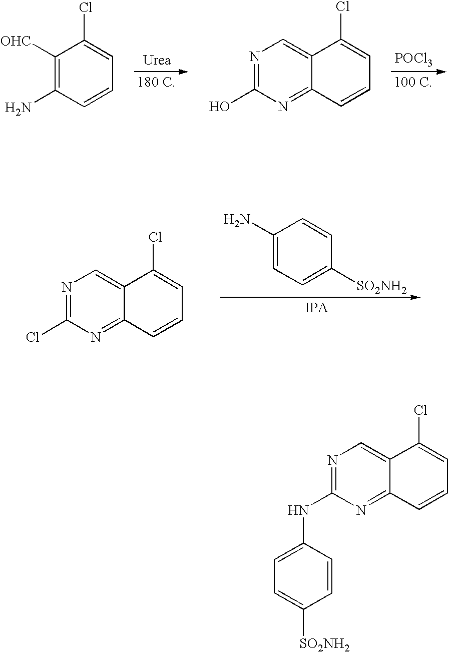

### 1. Preparation of 5-chloroquinazoli-2-ol

To 2-amino-6-chlorobenzaldehyde (1 eq) was added urea (10 eq) and the resulting mixture was heated to 180° C. for 3 h. Complete conversion to the product was observed by LC/MS. It was cooled and the product was filtered to get 5-chloroquinazoli-2-ol. ES/MS m/z 181 (MH⁺).

### 2. Preparation of 2,5 dichloroquinazoline

To 5-chloroquinazoli-2-ol was added phosphorusoxychloride and the mixture was heated to 110° C. for 3 h. Complete conversion to the product was observed by LC/MS. The reaction mixture was concentrated and poured in to ice-water and filtered and air dried to give 2,5 dichloroquinazoline. ES/MS m/z 196/198 (MH⁺).

### 3. Preparation of 4-(5-chloroquinazolin-2-ylamino)benzenesulfonamide

To 2,5 dichloroquinazoline (1 eq) in IPA was added sulfanilide (1.1 eq) and the mixture was heated to 110° C. for 16 h. Complete conversion to the product was observed. By LC/MS. It was filtered out to give 4-(5-chloroquinazolin-2-ylamino)benzenesulfonamide. ES/MS m/z 335/337 (MH⁺).

### Example 250

### (Table 1)

### Preparation of 1-(3-(6-bromoquinazolin-2-ylamino)-5-(1-methyl-1H-pyrazol-4-yl)benzyl)pyrrolidine-3-ol

The subject compound was prepared according to the general Scheme below:

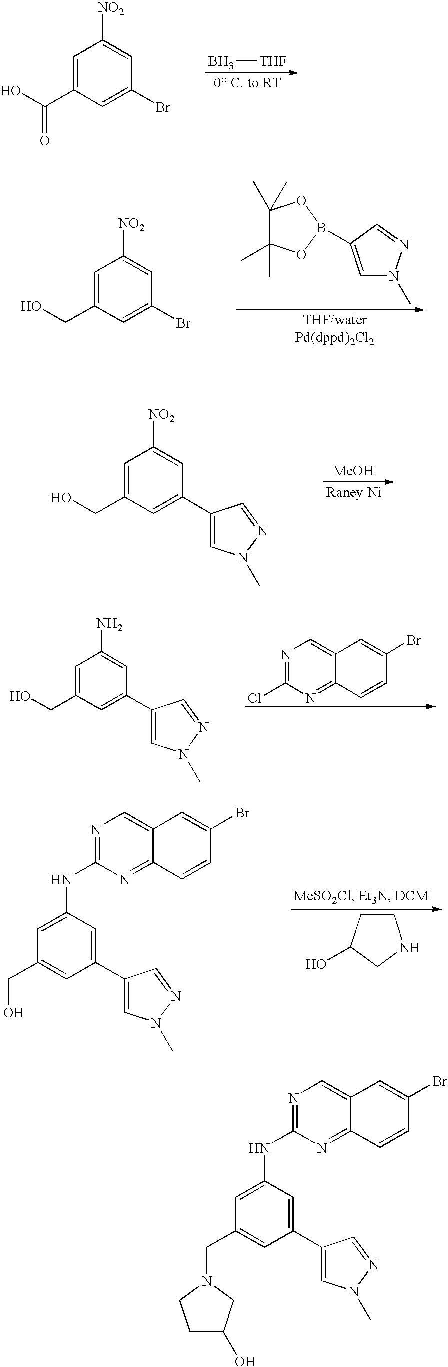

### 1. Preparation of (3-bromo-5-nitrophenyl)methanol

To a solution of 3-bromo5-nitrobenzoic acid (1 eq) in THF at 0° C. was added Borane-THF and the mixture was brought to ambient temperature and stirred for 16 h. Complete conversion to (3-bromo-5-nitrophenyl)methanol was observed by LC/MS. The reaction was cooled to 0° C. and was quenched with few drops of methanol and the mixture was concentrated. It was then partitioned between ethyl acetate and water. The organic layer was washed with brine and dried with sodium sulfate to give (3-bromo-5-nitrophenyl)methanol. ES/MS m/z 230 (MH⁺).

### 2. Preparation of (3-(1-methyl-1H-pyrazol-4-yl)-5-nitrophenyl)methanol

To a solution of (3-bromo-5-nitrophenyl)methanol in THF/water was added 1-methyl-4-(4,4,5,5-tetramethyl-1,3,2-dioxaborolan-2-yl)-1H-pyrazole (1.2 eq) and sodium carbonate (3 eq) and the mixture was degassed for 15 min. To it was added Pd(dppf)₂Cl₂ (0.05 eq) and the mixture was heated to 90° C. for 16 h. Complete conversion to the product was observed on LC/MS. The mixture was portioned between ethyl acetate and water and the organic layer was washed with brine and dried with sodium sulfate to give (3-(1-methyl-1H-pyrazol-4-yl)-5-nitrophenyl)methanol. ES/MS m/z 234 (MH⁺).

### 3. Preparation of (3-amino-5-(1-methyl-1H-pyrazol-4-yl)phenyl)methanol

To a solution (3-(1-methyl-1H-pyrazol-4-yl)-5-nitrophenyl)methanol in methanol was added Raney Ni under nitrogen and the mixture was hydrogenated for 5 h and observed the formation of (3-amino-5-(1-methyl-1H-pyrazol-4-yl)phenyl)methanol. The catalyst was filtered off to give the product. ES/MS m/z 204 (MH⁺).

### 4. Preparation of (3-(6-bromoquinazolin-2-ylamino)-5-(1-methyl-1H-pyrazol-4-yl)phenyl)methanol

To a solution of (3-amino-5-(1-methyl-1H-pyrazol-4-yl)phenyl)methanol (1.1 eq) in isopropanol was added 2-chloro-6-bromoquinazoline (1 eq) and the mixture was heated to 110° C. Complete conversion to the product was observed by LC/MS. The product was filtered and washed with isopropanol to give (3-(6-bromoquinazolin-2-ylamino)-5-(1-methyl-1H-pyrazol-4-yl)phenyl)methanol. ES/MS m/z 409/411 (MH⁺).

### 5. Preparation of 1-(3-(6-bromoquinazolin-2-ylamino)-5-(1-methyl-1H-pyrazol-4-yl)benzyl)pyrrolidin-3-ol

To a solution (3-(6-bromoquinazolin-2-ylamino)-5-(1-methyl-1H-pyrazol-4-yl)phenyl)methanol in dichloromethane at 0° C. was added triethylamine (3 eq) and methanesulfonylchloride (1.5 eq) and the mixture was brought to ambient temperature. To the reaction was added 3-pyrrolidinol (2 eq) and the mixture was stirred at ambient temperature for 16 h. Conversion to product was observed by LC/MS. The mixture was washed with brine and the organic layer was dried and concentrated to give 1-(3-(6-bromoquinazolin-2-ylamino)-5-(1-methyl-1H-pyrazol-4-yl)benzyl)pyrrolidine-3-ol. ES/MS m/z 479 (MH⁺).

### Example 251

### Synthesis of (2R,4S)-4-(2-(3-fluorophenylamino)-6-(thiazol-2-yl)quinazolin-7-yloxy)piperidine-2-yl)methanol

The subject compound was prepared according to the general Scheme below:

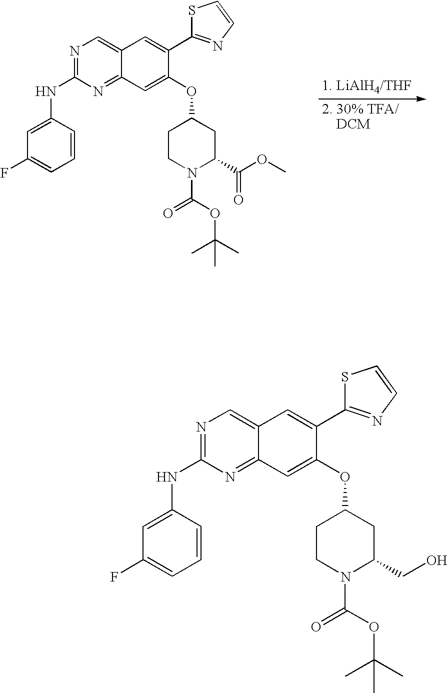

### Step 1. Preparation of (2R,4S)-tert-butyl-4-(2-(3-fluorophenylamino)-6-(thiazol-2-yl)quinazolin-7-yloxy-2-(hydroxymethyl)piperidine-1-carboxylate

To a stirred solution of Lithium Aluminium hydride (1.65 eq) in THF (1 ml) at 0° C. was added (2R,4S)-1-tert-butyl-2-methyl-4-(2-(3-fluorophenylamino)-6-(thiazol-2-yl) quinazolin-7-yloxy)piperidine-1,2-dicarboxylate (for synthesis, see example 997) (1 eq) in THF (1 ml). The reaction mixture was stirred 30 min at this temperature, then partitioned between water and ethylacetate. The organic layer was separated and washed with brine and dried over sodium sulfate. Filtered, evaporated and dried under vacuum to provide product in 70% yield. Proceed for next step without purification. ES/MS m/z 552.1 (MH⁺).

### Step 2. Preparation of (2R,4S)-4-(2-(3-fluorophenylamino)-6-(thiazol-2-yl)quinazolin-7-yloxy)piperidine-2-yl)methanol

The product from Step 1 was dissolved in 1 ml of 30% TFA:DCM and stirred for 30 min at ambient temperature and concentrated. The crude product was purified by reverse-phase HPLC and lyophilized to give the desired product as its trifluoroacetic acid salt. ES/MS m/z 452.1 (MH⁺).

### Example 252

### (Table 3): Synthesis of 4-((tert-butyldimethylsilyloxy)methyl)-2-(tributylstannyl)thiazole

The subject compound was prepared according to the general Scheme below:

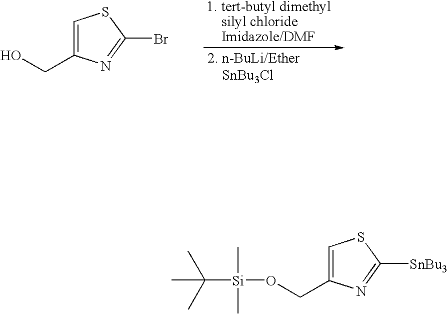

### Step 1. Preparation of 2-bromo-4-(tert-butylsilyloxy)methyl)thiazole

A mixture of 2-bromothiazol-4-yl)methanol (1 eq), imidazole (4 eq) and tert-butyl-dimethylsilylchloride (2 eq) in DMF(10 ml) was stirred 2 h at room temperature. The reaction mixture was partitioned between ethyl acetate and water. Ethylacetate layer was separated and washed with water, brine and dried over sodium sulfate. Filtered, evaporated and purified by flash chromatography (10% EtOAc/Hexane) to provide pure product as a colorless liquid in 95% yield ES/MS m/z 307.9/309.9 (MH⁺).

### Step 2. Preparation of 4-((tert-butyldimethylsilyloxy)methyl-2-(tributylstannyl)thiazole

To flame dried flask under nitrogen at −78° C. was added anhydrous ether (10 ml) and n-butyl lithium (1.5 eq, 2.5M solution in hexane) followed by addition of 2-bromo-4-(tert-butylsilyloxy)methyl) thiazole solution in ether (1 eq, 3 ml). Stirred at −78° C. for 1 h. Then a solution of tributyltinchloride in ether (1.5 eq, 1 ml) was added dropwise. Stirred at this temperature additional 1 h. The reaction mixture was quenched with satd. sodium bicarbonate and compound was extracted in ether. Ether extracts were combined, washed with brine and dried over sodium sulfate. Filtered, evaporated and dried under vacuum to provide product as a light yellow liquid. The crude product was obtained in quantitative yield. Used without further purification.

Compounds may be screened for activity in several in vitro assays as described below.

### I. PDK1 Kinase Alpha Screen Assay

Reagents/Concentrations: The PDK1-4 peptide substrate, biotin-GGGGRTWTLCG-NH₂, (SEQ ID NO: 1), is purchased from the Tufts University Core Facility. The final concentration of PDK1-4 peptide substrate is 50 nM. The ATP substrate (adenosine-5′-triphosphate) is purchased from Roche Diagnostics. The final concentration of ATP substrate is 10 μM. Phospho-(Ser/Thr) PKA substrate antibody is purchased from Cell Signaling Technology. The final concentration of antibody is 0.3 mg/mL. The Alpha Screen Protein A detection kit containing donor and acceptor beads is purchased from PerkinElmer Life Sciences. The final concentration of both donor and acceptor beads is 25 μg/mL. Alpha Screen is used for detection. The biotinylated-PDK1-4 peptide is phosphorylated by PDK1 kinase using the ATP substrate. The biotinylated-PDK1-4 peptide substrate is bound to the streptavidin coated donor bead. The antibody is bound to the protein A coated acceptor bead. The antibody bound to the phosphorylated form of the biotinylated PDK-1 peptide substrate, bringing the donor and acceptor beads into close proximity. Laser irradiation of the donor bead at 680 nm generates a flow of short-lived singlet oxygen molecules. When the donor and acceptor beads are in close proximity, the reactive oxygen generated by the irradiation of the donor beads initiates a luminescence/fluorescence cascade in the acceptor beads. This process leads to a highly amplified signal with output in the 530-620 nm range. Assays are carried out in 50 mM Tris, pH=7.5, 10 mM MgCl₂, 0.1% BSA, 0.01% Tween-20, 2 mM dithiolthreitol, 2.5% dimethyl sulfoxide. Reactions are stopped by adding 50 mM Tris, pH=7.5, 90 mM EDTA, 0.1% BSA, 0.01% Tween-20.

Procedure: To 10 μL of PDK1-4 peptide, 0.5 μl of test compound in dimethyl sulfoxide is added. PDK1 kinase and ATP are mixed, and 10 μL of the PDK1 kinase/ATP mix is added to start the reaction. The reaction is allowed to proceed for 3-18 hours. The reactions are then stopped by adding 10 μL of the EDTA-containing stop buffer. Beads were mixed with antibody, and 254 of the bead/antibody mix is then added to the stopped reactions. Plates are incubated at room temperature overnight to allow for detection development before being read. The assay is run in a 384-well format plate.

### II. CDK1 (CDC2) Kinase Inhibition In Vitro Screen Assay

Reagents/Concentrations: Human full length Cdk1 is purchased from Upstate (# 14-450) as a co-purification with Cyclin B. The final enzyme concentration in the assay is 0.8 nM. Histone H1 peptide substrate is purchased from Research Genetics. The peptide, with the sequence lcBiotin-GGCGPKTPKKAKKL[CONH₂], (SEQ ID NO: 2), is used in the assay at a final concentration 0.5 μM. The ATP substrate (Adenosine-5′-triphosphate) is purchased from Roche Diagnostics. The final concentration of ATP substrate is 1 μM. P³³ γ-ATP is purchased from NEN. The biotinylated peptide substrate is phosphorylated by Cdk1/Cyclin B enzyme, in the presence of varying concentrations of compounds, using the ATP substrate. A fraction of ATP in the reaction is radiolabeled to provide a detectable phosphorylation signal. The phosphorylation reaction is stopped with the addition of 25 mM EDTA. The solutions are then transferred to White BioBind Streptavidin Coated Assay plates, purchased from Thermo Electron Corporation. After washing, Microscint 20 scintillation fluid, purchased from Perkin Elmer, is added to each well and counts per minute (cpm) is measured using a Packard TopCount Microscintillation Counter. The highest cpms measured indicates the maximum phosphorylation of the substrate possible under the assay conditions. Reactions run without enzyme present give cpms indicative of complete inhibition of the enzyme. Each concentration of compound produces a measurable percent inhibition from the maximum signal based on these values. Assays are carried out in 50 mM Tris-HCl pH7.5, 10 mM MgCl₂, 1 mM DTT, 1 mM EGTA, 25 mM 3-glycerol phosphate, 1 mM NaF, 0.01% BSA/PBS, 0.5 μM peptide substrate, and 0.8 nM Cdk1.

Procedure: Reaction Buffer (100 μL) containing 50 mM Tris-HCl pH7.5, 10 mM MgCl₂, 0.01% BSA/PBS, 1.5 mM DTT, 1.5 mM EGTA, 37.5 mM β-glycerol phosphate, 1.5 mM NaF, 0.75 μM peptide substrate, and 1.2 nM Cdk1 is distributed to each well. 100% inhibition control wells contain no Cdk1. The compounds to be tested are added to wells in desired 10× concentrations with 10% DMSO, 50 mM Tris-HCl pH7.5, 10 mM MgCl₂, and 0.01% BSA/PBS. The reactions are initiated by adding 15 μL of ATP concentrated at 10 μM, with P³³ γ-ATP at <10 nM as label. The reactions are allowed to continue for four hours at room temperature with shaking. Streptavidin coated plates are blocked for one hour with 1% BSA in PBS. EDTA (100 μL, 50 mM) is added to each streptavidin well. An aliquot (100 μL) of each assay solution is transferred to the corresponding streptavidin well containing EDTA. The capture of radiolabeled substrate is then carried out by shaking the plate at room temperature for one hour. After binding the wells are washed 4 times with PBS, 200 μL Microscint 20 is added to each well, and cpms are measured. The assay is run in a 96-well format.

### III. CDK2 Kinase Inhibition In Vitro Screen Assay

Reagents/Concentrations: Human full length Cdk2 is purchased from Upstate (# 14-407) as a co-purification with Cyclin A. The final enzyme concentration in the assay is 5 nM. Histone H1 peptide substrate is purchased from Research Genetics. The peptide, with the sequence lcBiotin-GGCGPKTPKKAKKL[CONH₂], (SEQ ID NO: 2), is used in the assay at a final concentration 0.5 μM. The ATP substrate (adenosine-5% triphosphate) is purchased from Roche Diagnostics. The final concentration of ATP substrate is 1 μM. P³³ γ-ATP is purchased from NEN. The biotinylated peptide substrate is phosphorylated by Cdk2/Cyclin A enzyme, in the presence of varying concentrations of compounds, using the ATP substrate. A fraction of ATP in the reaction is radiolabeled to provide a detectable phosphorylation signal. The phosphorylation reaction is stopped by the addition of 25 mM EDTA. The solutions are then transferred to White BioBind Streptavidin Coated Assay plates, purchased from Thermo Electron Corporation. After washing, Microscint 20 scintillation fluid, purchased from Perkin Elmer, is added to each well and counts per minute (cpm) are measured using a Packard TopCount Microscintillation Counter. The highest cpms measured indicate the maximum phosphorylation of the substrate possible under the assay conditions. Reactions run without enzyme present give cpms indicative of complete inhibition of the enzyme. Each concentration of compound produces a measurable percent inhibition from the maximum signal based on these values. Assays are carried out in 50 mM Tris-HCl pH7.5, 10 mM MgCl₂, 1 mM DTT, 1 mM EGTA, 25 mM β-glycerol phosphate, 1 mM NaF, 0.01% BSA/PBS, 0.5 μM peptide substrate, and 5 nM Cdk1.

Procedure: Reaction Buffer (100 μL) containing 50 mM Tris-HCl pH7.5, 10 mM MgCl₂, 0.01% BSA/PBS, 1.5 mM DTT, 1.5 mM EGTA, 37.5 mM β-glycerol phosphate, 1.5 mM NaF, 0.75 μM peptide substrate, and 7.5 nM Cdk2 is distributed to each well. 100% inhibition control wells contain no Cdk2. The compounds to be tested are added to wells in desired 10× concentrations with 10% DMSO, 50 mM Tris-HCl pH7.5, 10 mM MgCl₂, and 0.01% BSA/PBS. The reactions are initiated by adding 15 μL of ATP concentrated at 10 μM, with P³³ γ-ATP at <10 nM as label. The reactions are allowed to proceed for four hours at room temperature with shaking. Streptavidin coated plates are blocked for one hour with 1% BSA in PBS. EDTA (100 μL, 50 mM) is added to each streptavidin well. An aliquot (100 μL) of each assay solution is transferred to corresponding streptavidin wells containing EDTA. The radiolabeled substrate is captured by shaking at the plate at room temperature for one hour. After binding, the wells are washed 4 times with PBS, 200 μL Microscint 20 is added to each well, and cpms are measured. The assay is run in a 96-well format plate.

### IV. Cell Proliferation Assay Protocol: A2780 of PC-3 Cell Line

A2780 or PC-3 cells are seeded at 1000 cells/well in 100 μL/well (10.000 cells/mL) growth media in 96-well plates. Cells are allowed to adhere to the bottom of plates for 3-5 hours in a 37° C. 5% CO₂ incubator. Compounds are dissolved in DMSO and then transferred to the cell plates. The cells are incubated with the compounds for 3 days in a 37° C. 5% CO₂ incubator. The growth medium containing the compounds is then removed from the cells and fresh medium is added, followed by 100 μl, of Cell Titer Glo assay reagent (Promega). This mixture is shaken for 1 minute and then incubated without shaking for 10 minutes. Activity determinations for the compounds are made by detection on a Trilux Instrument.

### V. Cell Proliferation Assay Protocol: PC-3 Cell Line

PC-3 cells are seeded at 1000 cells/well in 100 μL/well (10.000 cells/mL) along with growth media into black-walled, clear bottom 96-well plates. The cells are allowed to adhere to the bottom of the plate for 3-5 hours in a 37° C. 5% CO₂ incubator.

Test compounds are diluted to 500× in DMSO. The DMSO solutions of six of the compounds are transferred to the cells in the 96 well round bottom plate, column 2, row B-F.

A 1:3 serial dilution of each compound is carried out. The serial dilution comprised adding 20 μL of DMSO to the wells containing the compounds and doing a 1:3 dilution across the plate from columns 2-10. Column 11 contained only DMSO. The serial dilution is carried out using a BioMek 2000 protocol “CP Serial Dilution using 250 μL tips” or “Proliferation Compound” (if using 20 μL tips).

To a 96 deep well block, columns 2-11 rows B-F, is transferred 500 μL of growth medium. Using the FX protocol “HH_CellAssay_{—}2 μL to 500 μL”, 2 μl, of compound from each cell of the compound plate is transferred to the corresponding cell in the 96 deep well block containing 500 μL of growth medium. The instrument is programmed to dilute the compound in growth medium and then transfer 100 μL of that mixture to cell plates containing cells.

The cell plates, to which test compounds had been added, are incubated for 3 days at 37° C. Following the incubation, the medium is removed and replaced with fresh medium. Cell Titer Glo (100 μL) is added to each well and the plate is shaken for 1 minute and then incubated without shaking for 10 minutes. The plates are then read using a Trilux instrument.

VI. The pAkt^{T308} ECL Assay Protocol

On Day 1, PC-3 cells are seeded at 15,000 cells/well in 100 μL/well (10.000 cells/mL) growth media into black-walled, clear bottom, poly-L-lysine coated plates. The cells are incubated overnight in a 37° C., 5% CO₂ incubator.

On Day 2, a MSD ECL plate is blocked for two hours with 150 μL per well of 3% MSD blocker A.

Test compounds are diluted to 500× in DMSO and then are subjected to further serial dilution using a BioMek 2000 instrument. DMSO diluted compounds are then diluted into growth media and then added to the cell plates.

The cell plates incubated with compounds for six hours in a 37° C., 5% CO₂ incubator after which the growth medium is removed and 55 μl of MSD lysis buffer is added to cell plates on ice. The plates are lysed on ice for five minutes followed by 15 minutes of vigorous shaking on a plate shaker at 4° C. The blocked MSD assay plates are washed twice with 1×MSD wash buffer followed by the addition of cell lysate as follows: 30 μl of cell lysate is added to the pAkt308 plates and 13 μl of lysate+12 μl lysis buffer is added to the tAkt plates. The plates are then sealed and shaken at 4° C. overnight.

On Day 3, the MSD plates are washed four times with 1×MSD wash buffer then, 25 μl/well of MSD SULFO-TAG antibodies diluted to 10 nM final concentration in 1% blocker. A buffer is added to the antibody diluent which is added to assay plates. The plates are then sealed and incubated at RT for 1.5 hour. The plates are then washed twice with 1×MSD wash buffer followed by the addition of 150 μl/well of 1.5×MSD read buffer. The plates are read immediately after the addition of read buffer using a Trilux instrument.

Accordingly, certain compounds of the invention were screened in the PDK1 kinase alpha screen assay (I, above; fourth column of Table 1), the pAkt^{T308} ECL Assay Protocol (VI above; data in the fifth column of Table 1), and the cell proliferation assay protocol, A2780 of PC-3 cell line (IV above) or the cell proliferation assay protocol, PC-3 cell line (V above) (data for either protocol in the sixth column of Table 1). Data is shown for certain compounds of the invention in Table 1. The symbol “A” indicates ICₛₒ values of less than 2 μM or greater, the symbol “B” indicates IC₅₀ values of 2 to 5 μM, the symbol “C” indicates IC₅₀ values of 5 to 10 μM, and the symbol “D” indicates IC₅₀ values greater than 10 μM. Table 1 also shows the actual molecular weight of each compound (MW) and the retention time (rt, minutes) and mass spectral (m/z) data for each compound, where measured.

The compounds of the present invention may also be prepared by methods analogous to those used to prepare the compounds of PCT/US2007/088392, filed Dec. 20, 2007 (which is hereby incorporated by reference in its entirety), but starting from appropriate starting materials. The experimental disclosure of PCT/US2007/088392 follows:

### Example 1

### (Table 4): 4-(6-Bromo-8-methoxyquinazolin-2-ylamino)benzene-sulfonamide

The subject compound was prepared according to the general Scheme 1, below:

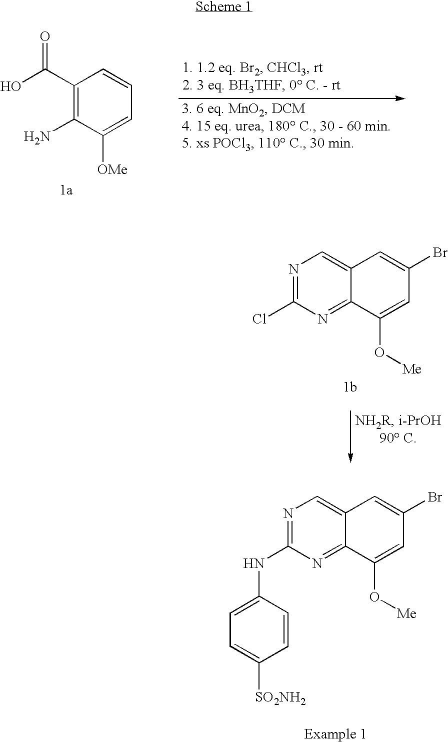

### Step 1: 2-Amino-5-bromo-3-methoxybenzoic acid

To a 0.24 M chloroform solution of 2-amino-3-methoxybenzoic acid (4, 11.87 g, 71.7 mmol) at 0° C. was added bromine (1.08 eq. 0.31 M) in chloroform dropwise. The mixture was warmed to room temperature (RT) and stirred under argon for 16 hours. A precipitate formed and was collected by filtration and washed thoroughly with chloroform. The crude material was dried in vacuo to give the title as a hydrobromide (HBr) salt in 99% yield. ES/MS m/z 248/250 (MH⁺).

### Step 2: (2-Amino-5-bromo-3-methoxyphenyl)methanol

To a 0.24 M tetrahydrofuran (THF) suspension of 2-amino-5-bromo-3-methoxybenzoic acid, 1a (71.7 mmol) at 0° C. was added borane THF solution (1 M, 220 mL, 220 mmol). The mixture was stirred under argon at RT for 66 hours. The reaction was quenched by adding ethanol (15 mL) at 0° C. and stirred for 15 minutes. The mixture was poured into water and extracted with DCM. The organic extracts were combined, washed with brine, dried with sodium sulfate and concentrated in vacuo to give crude material as a white solid (10.16 g, 62% yield). ES/MS m/z 230/232 (MH⁺).

### Step 3: 2-Amino-5-bromo-3-methoxybenzaldehyde

To a 0.15 M chloroform solution of (2-amino-5-bromo-3-methoxyphenyl)methanol (10.16 g, 43.96 mmol) was added manganese dioxide (19.9 g, 280.5 mmol). The mixture was stirred under argon at RT for 16 hours. The mixture was then filtered through diatomaceous earth and washed with DCM. The filtrate was concentrated to dryness and used in next step. ES/MS m/z 228/230 (MH⁺).

### Step 4: 6-Bromo-8-methoxyquinazolin-2-ol, 1a

The mixture of 2-amino-5-bromo-3-methoxybenzaldehyde (43.96 mmol, crude material from Step 3) and urea (35 g, 583 mmol) from the previous step was heated to 180° C. under argon for 1 hour. Water (300 mL) was added after cooling to RT. A solid precipitate formed and was collected by filtration and air dried to give 12.45 g of a powder. ES/MS m/z 254/256 (MH⁺).

### Step 5: 6-Bromo-2-chloro-8-methoxyquinazoline, 1b

A suspension of 6-bromo-8-methoxyquinazolin-2-ol, 1a (43.96 mmol) in POCl₃ (120 mL) was heated to 110° C. for 30 minutes. The mixture was cooled to RT, evaporated POCl₃ and partitioned between water and DCM. The organic portion was concentrated to give a crude material which was purified by column chromatography (silica gel, eluted with 2% MeOH in DCM) to yield a pure material as a yellow solid in 30% yield (3 steps, 3.62 g). ES/MS m/z 272/274 (MH⁴).

### Step 6: 4-(6-Bromo-8-methoxyquinazolin-2-ylamino)benzenesulfonamide

To a solution of 50 mg of 1b in 2-propanol (1 mL) was added sulfanilamide (1.0 eq). The reaction was stirred at 90° C. for 18 hours. The hydrochloride was collected by vacuum filtration and air dried to give a crude material (80 mg) which can be used for further chemical modifications. The pure material was obtained by HPLC purification. ES/MS m/z 409/411 (MH⁺).

### Example 2

### (Table 4): 4-(6-Ethynyl-8-methoxyquinazolin-2-ylamino)benzenesulfonamide

The subject compound was prepared according to the general Scheme 2, below:

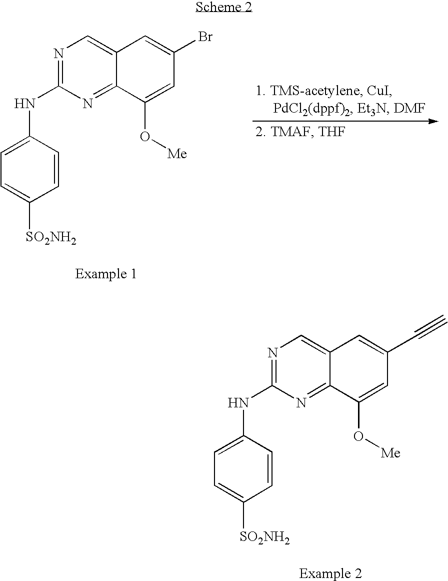

Step 1 and Step 2 were carried out in one pot. A mixture of the starting material (4-(6-bromo-8-methoxyquinazolin-2-ylamino)benzenesulfonamide; synthesized following Example 1, 67 mg), ethynyltrimethylsilane (0.12 mL), copper(I) iodide (6 mg), 1,1′-bis(diphenylphosphino)ferrocenedichloro palladium(II) (12 mg), TEA (0.8 mL) and DMF (0.8 mL) was microwaved at 120° C. for 6 min. LC/MS showed complete conversion of starting material to 4-(8-methoxy-6-((trimethylsilyl)ethynyl)quinazolin-2-ylamino)benzenesulfonamide. To this intermediate was added THF (0.8 mL) and tetramethylammonium fluoride (60 mg). The resulting mixture was stirred at RT for 16 hrs. The mixture was then diluted with water and extracted with ethyl acetate (3×). The organic extracts were combined, washed with brine, dried with sodium sulfate and concentrated to give a crude material which was purified by HPLC to yield the title compound 4-(6-ethynyl-8-methoxyquinazolin-2-ylamino)benzenesulfonamide. ES/MS m/z 355 (MH⁺).

### Example 3

### (Table 4): 4-(6-Ethyl-8-methoxyquinazolin-2-ylamino)benzenesulfonamide

The subject compound was prepared according to the general Scheme 3, below:

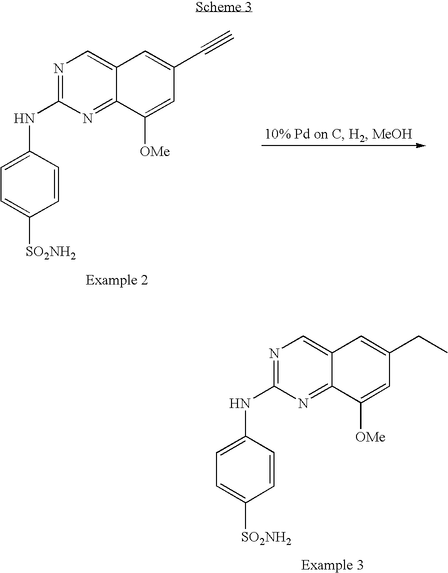

To a 0.015M solution of the product of Example 2 (1.0 eq) in MeOH was added 10% palladium on carbon (20% by mass). The reaction vessel was evacuated and flushed with hydrogen gas. The resulting mixture was stirred 14 h at ambient temperature and then filtered through diatomaceous earth and concentrated. The crude product was purified by reverse-phase HPLC, and lyophilized to give the desired compound as its trifluoroacetic acid salt. ES/MS m/z 359 (MH⁺).

### Example 4

### (Table 4): 4-(6-Cyano-8-methoxyquinazolin-2-ylamino)benzenesulfonamide

The subject compound was prepared according to the general Scheme 4, below:

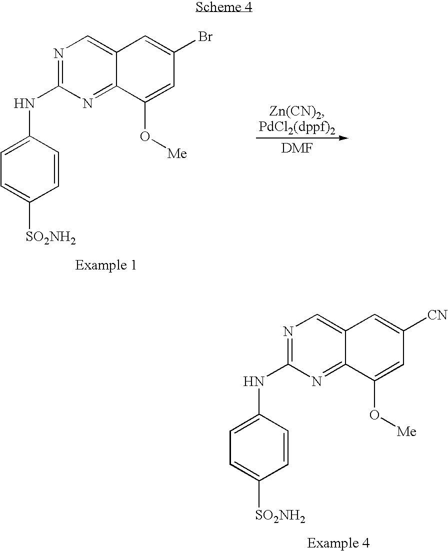

A mixture of 4-(6-bromo-8-methoxyquinazolin-2-ylamino)benzenesulfonamide (synthesized following Example 1, 36 mg), zinc(II) cyanide (36 mg), 1,1′-bis(diphenyl-phosphino)ferrocenedichloro palladium(II) (12 mg) and DMF (1 mL) was microwaved at 120° C. for 22 min. The resulting mixture was diluted with ethyl acetate, washed with water and brine, dried with sodium sulfate and concentrated. The crude material was purified by HPLC to give the pure title compound 4-(6-cyano-8-methoxyquinazolin-2-ylamino)benzenesulfonamide. ES/MS m/z 356 (MH⁺).

### Example 5

### (Table 5): 4-(8-Methoxy-6-methylquinazolin-2-ylamino)benzenesulfonamide

The subject compound was prepared according to the general Scheme 5, below:

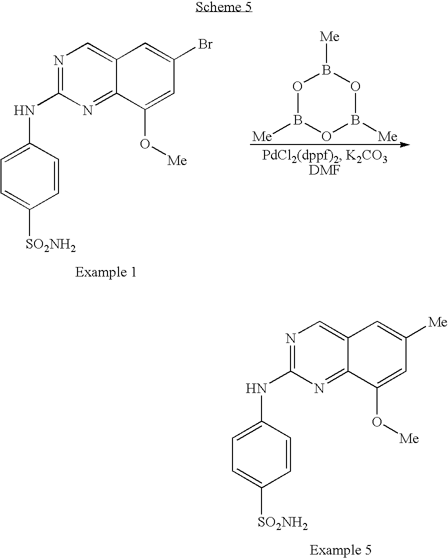

A mixture of 4-(6-bromo-8-methoxyquinazolin-2-ylamino)benzenesulfonamide (synthesized following Example 1, 20 mg), trimethylboroxine (20 μL), 1,1′-bis(diphenyl-phosphino)ferrocenedichloro palladium(II) (12 mg), potassium carbonate (0.6 mL of 2 M aqueous solution) and DMF (1.2 mL) was microwaved at 130° C. for 10 min. The resulting mixture was diluted with ethyl acetate, washed with water and brine, dried with sodium sulfate and concentrated. The crude material was purified by HPLC to give the pure title compound 4-(8-methoxy-6-methylquinazolin-2-ylamino)benzenesulfonamide. ES/MS m/z 345 (MH⁺).

### Example 6

### (Table 4): N-(3-(6-Bromo-8-chloroquinazolin-2-ylamino)-5-((dimethylamino)methyl)phenyl)acetamide

The subject compound was prepared according to the general Scheme 6, below:

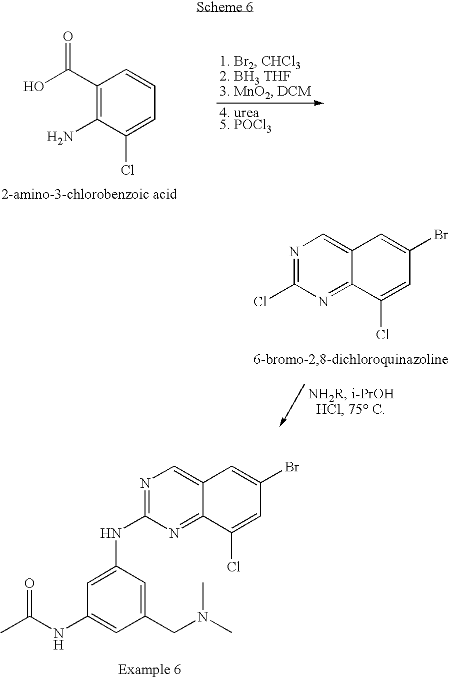

### Step 1

To a suspension of 2-amino-3-chlorobenzoic acid (2 g, 11.6 mmol) in chloroform (120 mL) was added dropwise bromine (1.1 equiv.) in chloroform (12 mL) solution. The mixture was stirred at RT for 16 hrs. The resulting white solid was collected by filtration and washed thoroughly with DCM until the filtrate was colorless. The solid was air-dried to give 3.35 g of white powder as HBr salt of 2-amino-5-bromo-3-chlorobenzoic acid (87% yield). ES/MS m/z 250/252 (MH⁺).

### Step 2

To the above intermediate (3.35 g, 10.1 mmol) in THF (40 mL) at 0° C. was added borane-THF complex solution (1 M in THF, 40 mL, 4 equiv.). The mixture was stirred at RT for 18 hrs. Additional borane-THF (20 mL) was added and continued reaction for another 24 hrs until the complete conversion of the starting material. The solvent was removed in vacuo and the excess reagent was quenched by addition of ethanol (20 mL) slowly. Water was added and the pH (˜3) was adjusted by adding sodium bicarbonate (sat. aq.) to pH 7. The resulting mixture was extracted with DCM. The organic extracts were combined, washed with brine, dried with sodium sulfate and concentrated to give a crude material as white solid. ES/MS m/z 236/238 (MH⁺).

### Step 3

To the above intermediate (10.1 mmol) in DCM (80 mL) was added manganese dioxide (MnO₂, 6 g, 70 mmol). The mixture was stirred at RT under argon for 40 hrs. Additional manganese dioxide (6 g) was added and the reaction was continued for another 20 hrs until the complete conversion of the starting material. The mixture was filtered through diatomaceous earth and washed thoroughly with DCM: The filtrate was concentrated in vacuo to give a crude product, (2-amino-5-bromo-3-chlorophenyl)methanol (3.3 g, orange solid) which was used for the next step without further purification. ES/MS m/z 234/236 (MH⁺).

### Step 4

A mixture of (2-amino-5-bromo-3-chlorophenyl)methanol (3.3 g, obtained from Step 3) and urea (10.5 g, 15 equiv.) was heated to 180° C. with vigorous stirring for 1 hr. The reaction was cooled to RT and water was added. The solid was collected by filtration. The filtered solid was air-dried to give 2-hydroxyquinazoline as a yellow powder (2.18 g, crude). ES/MS m/z 259/261 (MH⁺).

### Step 5: 6-Bromo-2,8-dichloroquinazoline

To the above crude material was added phosphorus oxychloride (POCl₃, 25 mL) and heated to 130° C. for 30 min. The resulting mixture was cooled to RT and concentrated in vacuo to nearly dryness. Ice water was added and pH was adjusted to ˜8 using sodium bicarbonate. The mixture was extracted with DCM and the extract was dried with sodium sulfate and concentrated in vacuo yielded desired product 6-bromo-2,8-dichloroquinazoline as brown foam (1.4 g). This material was used in the following step and in other chemical medications without further purification.

### Step 6: N-(3-(6-Bromo-8-chloroquinazolin-2-ylamino)-5-((dimethylamino)methyl)-phenyl)acetamide

A mixture of 6-bromo-2,8-dichloroquinazoline (0.175 g), N-(3-amino-5-((dimethylamino)methyl)phenyl)acetamide (1 equiv.) and HCl in dioxane (1 equiv.) in isopropanol (2.5 mL) was heated to 75° C. for 16 hrs. The resulting mixture was diluted with water, washed with ethyl acetate to remove organic impurities, basified the aqueous portion with sodium bicarbonate (aq.) to pH 9, and then brine was added. The basified aqueous solution was extracted with chloroform (3×). The organic extracts were combined, washed with brine, dried with sodium sulfate and concentrated to give a crude material which was used for the next step. Purification by HPLC then yielded pure N-(3-(6-bromo-8-chloroquinazolin-2-ylamino)-5-((dimethylamino)methyl)phenyl)acetamide. ES/MS m/z 448/450 (MH⁺).

### Example 7

### (Table 4): N-(3-(8-Chloro-6-ethynylquinazolin-2-ylamino)-5-((dimethylamino)methyl)phenyl)acetamide

The subject compound was prepared according to the general Scheme 7, below:

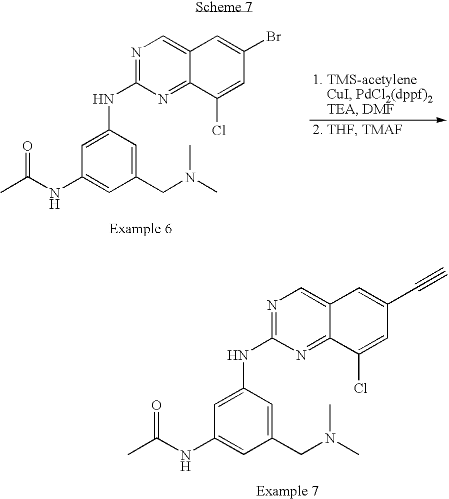

### Step 1

A mixture of N-(3-(6-bromo-8-chloroquinazolin-2-ylamino)-5-((dimethylamino)-methyl)phenyl)acetamide (63 mg, from Example 6), ethynyltrimethylsilane (0.063 mL), copper(I) iodide (6 mg), 1,1′-bis(diphenylphosphino)ferrocenedichloro palladium(II) (12 mg), TEA (0.8 mL) and DMF (0.8 mL) was microwaved at 120° C. for 8 min. The resulting mixture was diluted with ethyl acetate, washed with water and brine, dried with sodium sulfate and concentrated to give a dark brown residue.

### Step 2: N-(3-(8-Chloro-6-ethynylquinazolin-2-ylamino)-5-((dimethylamino)methyl)-phenyl)acetamide

To the intermediate from Step 1 was added THF (3 mL), 2-propanol (0.4 mL) and tetramethylammonium fluoride (18 mg). The mixture was stirred at RT for 20 min. The resulting mixture was diluted with water and extracted with ethyl acetate (3×). The organic extracts were combined, washed with brine, dried with sodium sulfate and concentrated to give a crude material which was purified by HPLC to yield the title compound N-(3-(8-chloro-6-ethynylquinazolin-2-ylamino)-5-((dimethylamino)methy)-phenyl) acetamide. ES/MS m/z 394 (MH⁺).

### Example 8

### (Table 4): 4-(8-Bromo-6-(trifluoromethyl)quinazolin-2-ylamino)benzenesulfonamide

The subject compound was prepared according to the general Scheme 8, below:

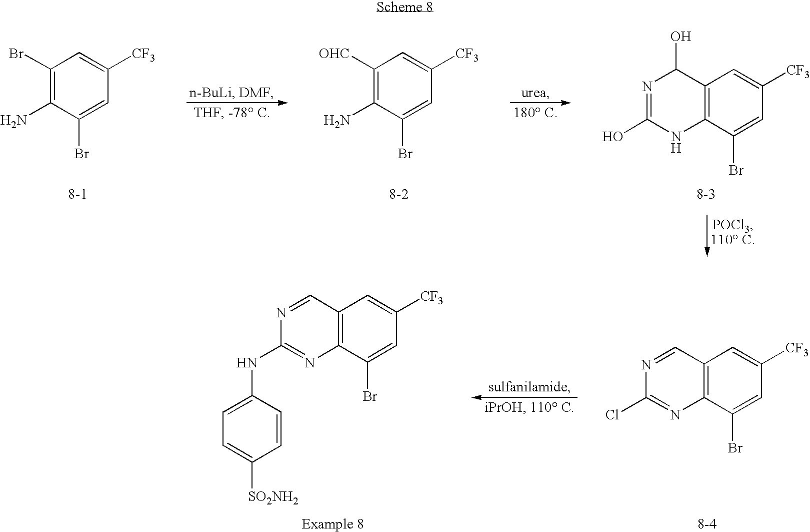

### Step 1: 2-Amino-3-bromo-5-(trifluoromethyl)benzaldehyde, 8-2

2,6-Dibromo-4-(trifluoromethyl)aniline, 8-1 (3.19 g, 10.0 mmol, 1.00 eq) was dissolved in THF (50 mL) and cooled to −78° C. A 2.5 M solution of n-butyllithium in hexanes (8.40 mL, 21.0 mmol, 2.10 eq) was added dropwise over 15 min. The mixture was stirred at −78° C. for 1 h. A solution of DMF (1.03 mL, 14.0 mmol, 1.40 eq) in THF (5 mL) was added, and the mixture was stirred an additional 1 h at −78° C. The reaction was allowed to come to −15° C. over 30 min and then quenched with brine. The mixture was diluted with ethyl acetate, washed sequentially with water and brine, dried over sodium sulfate, filtered, and concentrated to give 1.74 g of the desired product as a pale yellow, crystalline solid. ES/MS m/z 268,270 (MN).

### Step 2: 8-Bromo-6-(trifluoromethyl)-1,4-dihydroquinazoline-2,4-diol, 8-3

2-Amino-3-bromo-5-(trifluoromethyl)benzaldehyde, 8-2 (1.74 g, 6.49 mmol, 1.00 eq) and urea (5.85 g, 97.4 mmol, 15.0 eq) were stirred at 190° C. for 3 h. The resulting solid was returned to ambient temperature, stirred in water (60 mL) for 20 min, and filtered. This was repeated for a total of three washes. The solid was dried in a desiccator to give 3.79 g of the desired product as an off-white solid. ES/MS m/z 311, 313 (MH⁺).

### Step 3: 8-Bromo-2-chloro-6-(trifluoromethyl)quinazoline, 8-4

8-Bromo-6-(trifluoromethyl)-1,4-dihydroquinazoline-2,4-diol, 8-3 (3.79 g, 6.49 mmol, 1.00 eq) and phosphorus oxychloride (20 mL) were stirred together at 110° C. for 1.5 h. Volatiles were removed under reduced pressure. Ice water was added, and the pH was adjusted to 6-7 with aqueous sodium hydroxide and sodium bicarbonate. The precipitate was filtered off, rinsed with water, and dried under high vacuum. The crude material was triturated with THF. The mother liquor was concentrated to yield 332 mg of the desired product as an orange, crystalline solid. ES/MS m/z 313 (MH⁺).

### Step 4: 4-(8-bromo-6-(trifluoromethyl)quinazolin-2-ylamino)benzenesulfonamide

To a 0.30 M solution of 8-bromo-2-chloro-6-(trifluoromethyl)quinazoline, 8-4, in 2-propanol was added sulfanilamide (1.0 eq). The reaction was stirred at 110° C. for 14 h. The hydrochloride was collected by vacuum filtration and then stirred in aqueous sodium bicarbonate. The solid was collected by vacuum filtration and rinsed with water. The light yellow solid was dried in a desiccator to give 343 mg of the desired product. ES/MS m/z 447,449 (MH⁺).

### Example 9

### (Table 4): 4-(6-Bromoquinazolin-2-ylamino)benzenesulfonamide

The subject compound was prepared according to the general Scheme 9, below:

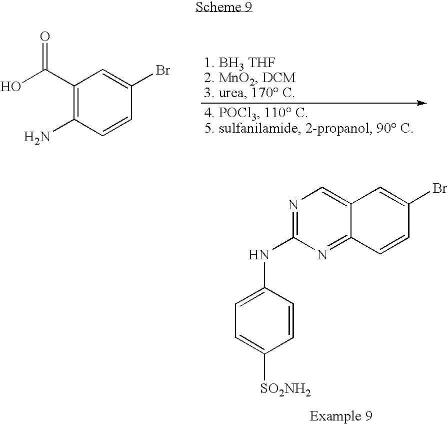

### Step 1: 2-Amino-5-bromobenzylalcohol

To a 0.50M solution of 2-amino-5-bromobenzoic acid (1.0 eq) in THF was added a 1.0 M solution of borane-THF complex in THF (2.0 eq) dropwise over 15 min at 0° C. The mixture was stirred for 2 d at ambient temperature. The reaction was quenched by the sequential addition ethanol (6.0 eq) and water. The mixture was extracted with ethyl acetate. The combined extracts were washed with brine, dried over sodium sulfate, filtered, and concentrated to give the desired product. ES/MS m/z 202,204 (MH⁺).

### Step 2: 2-Amino-5-bromobenzaldehyde

Manganese (IV) oxide (6.0 eq) was added to a 0.20M solution of 2-amino-5-bromobenzyl alcohol (1.0 eq) in DCM. The mixture was stirred at ambient temperature for 16 h and then filtered through Celite. The filtrate was concentrated to give the desired product as an orange-brown, crystalline solid. ES/MS m/z 200,202 (MH⁺).

### Step 3: 6-Bromo-2-hydroxyquinazoline

2-Amino-5-bromobenzaldehyde (1.0 eq) and urea (8.0 eq) were stirred at 170° C. for 1 h. The resulting solid was returned to ambient temperature, stirred in water for 20 min, and filtered. This was repeated for a total of three washes. The solid was dried in a desiccator to give the desired product as an off-white solid. ES/MS m/z 225,227 (MH⁺).

### Step 4: 6-Bromo-2-chloroquinazoline

A 0.50M solution of 6-bromo-2-hydroquinazoline in phosphorus oxychloride was stirred at 110° C. for 1.5 h. Volatiles were removed under reduced pressure. Ice water was added, and the pH was adjusted to 6-7 with aqueous sodium hydroxide and sodium bicarbonate. The precipitate was filtered off, rinsed with water, and dried under high vacuum to yield the desired product as a yellow solid. ES/MS m/z 245 (MH⁺).

### Step 5: 4-(6-Bromoquinazolin-2-ylamino)benzenesulfonamide

To a 0.50 M solution of 6-bromo-2-chloroquinazoline in 2-propanol was added sulfanilamide (1.0 eq). The reaction was stirred at 90° C. for 14 h. The hydrochloride was collected by vacuum filtration and used without further purification. Alternatively, the crude reaction mixture was concentrated, purified by reverse-phase HPLC, and lyophilized to give the desired compound as its trifluoroacetic acid salt. ES/MS m/z 379,381 (MH⁺).

### Example 10

### (Table 4): 4-(6-Ethynylquinazolin-2-ylamino)benzenesulfonamide

The subject compound was prepared according to the general Scheme 10, below:

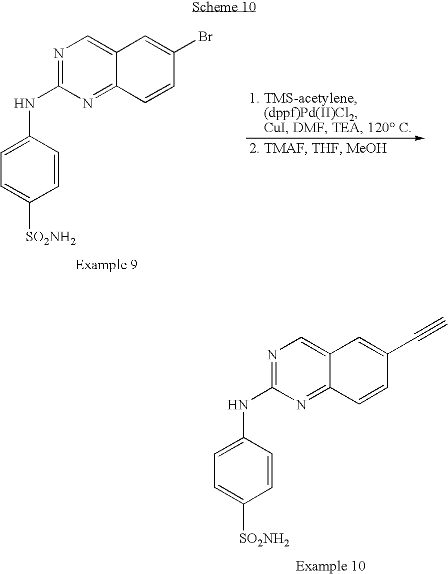

### Step 1

To a 0.15 M solution of the product of Example 9 in 1:1 DMF:TEA was added trimethylsilylacetylene (TMS-acetylene) (4.0 eq); copper(I) iodide (0.10 eq); and [1,1′-bis(diphenylphosphino)ferrocene]dichloropalladium(II) complex with DCM (0.050 eq). The reaction was microwaved at 120° C. for 10 min. The mixture was diluted with ethyl acetate and filtered through a pad of silica gel. The filtrate was concentrated and used without further purification. ES/MS m/z 397 (MH⁺).

### Step 2: 4-(6-Ethynylquinazolin-2-ylamino)benzenesulfonamide

To a 0.10 M solution of the product of Step 1 in 1:1 THF:MeOH was added tetramethylammonium fluoride (1.5 eq). The reaction was stirred for 30 min at ambient temperature. Volatiles were removed under reduced pressure, and the residue was partitioned between ethyl acetate and saturated aqueous sodium bicarbonate. The organic phase was dried over sodium sulfate, filtered, and concentrated. The crude material was purified by reverse-phase HPLC, and lyophilized to give the desired compound as its trifluoroacetic acid salt. ES/MS m/z 325 (MH⁺).

### Example 11

### (Table 4): 4-(6-Bromoquinazolin-2-ylamino)-N-isopropylbenzamide

The subject compound was prepared according to the general Scheme 11, below:

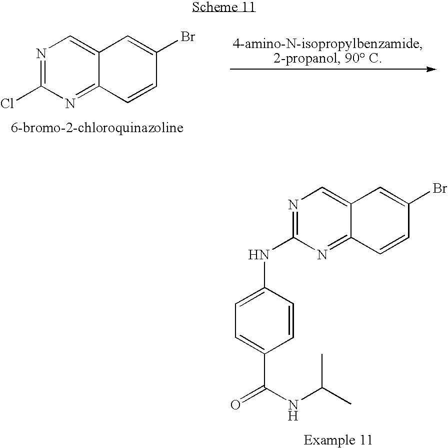

The procedure is analogous to Example 9 Step 5, using 4-amino-N-isopropyl-benzamide in place of sulfanilamide. ES/MS m/z 385,387 (MH⁴).

### Example 12

### (Table 4): N-Isopropyl-4-(6-(thiazol-2-yl)quinazolin-2-ylamino)-benzamide

The subject compound was prepared according to the general Scheme 12, below:

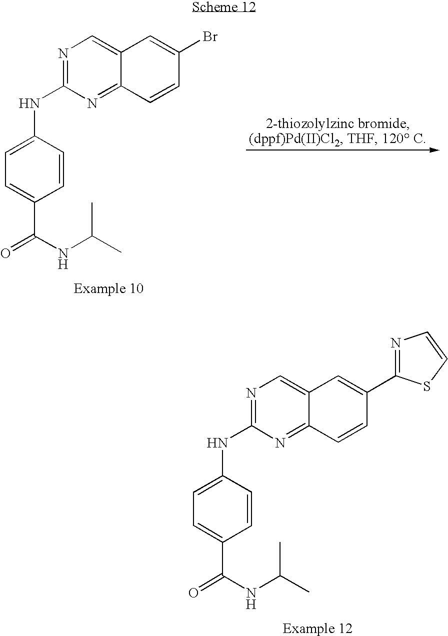

To the product of Example 10 was added [1,1′-bis(diphenylphosphino)ferrocene]-dichloropalladium(II) complex with DCM (0.10 eq) and a 0.5M solution of 2-thiazolyl zinc bromide in THF (3.0 eq). The reaction was microwaved at 120° C. for 10 min. The mixture was then diluted with ethyl acetate and washed with aqueous EDTA pH˜9 buffer. The organic phase was dried over sodium sulfate, concentrated, purified by reverse-phase HPLC, and lyophilized to give the desired compound as its trifluoroacetic acid salt. ES/MS m/z 390 (MH⁺).

### Example 13

### (Table 4): 4-(6-Cyanoquinazolin-2-ylamino)-N-isopropylbenzamide

The subject compound was prepared according to the general Scheme 13, below:

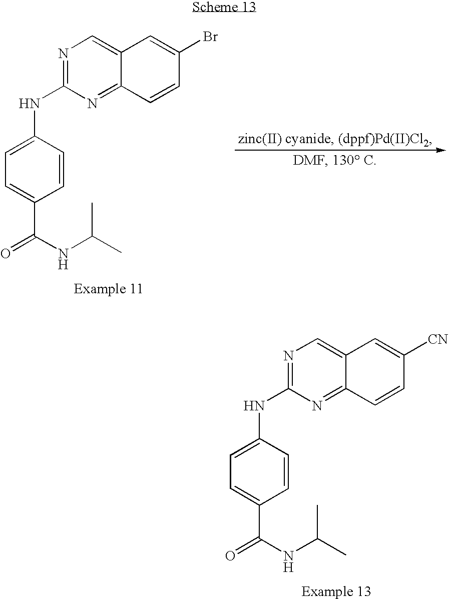

To a 0.10 M solution of the product of Example 11 in DMF was added zinc(II) cyanide (4.0 eq) and [1,1′-bis(diphenylphosphino)ferrocene]dichloropalladium(II) complex with DCM (0.10 eq). The reaction was microwaved at 130° C. for 10 min. The mixture was diluted with ethyl acetate and washed with aqueous EDTA pH˜9 buffer. The organic phase was dried over sodium sulfate, concentrated, purified by reverse-phase HPLC, and lyophilized to give the desired compound as its trifluoroacetic acid salt. ES/MS m/z 332 (MH⁺).

### Example 14

### (Table 4): N-(3-(6-Bromo-5-chloro-8-methoxyquinazolin-2-ylamino)-5-((dimethylamino)methyl)phenyl)acetamide

The subject compound was prepared according to the general Scheme 14, below:

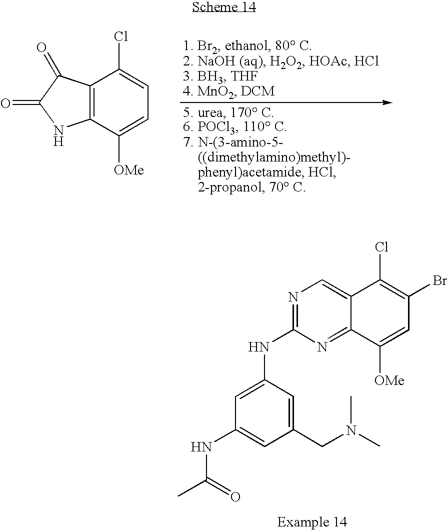

### Step 1: 5-Bromo-4-chloro-7-methoxyisatin

To a 0.50M solution of 4-chloro-7-methoxyisatin (1.0 eq) in ethanol at 80° C. was added a 1.0M ethanolic solution of bromine (2.0 eq) over 45 min. The reaction was stirred at 70° C. for 14 h and then concentrated. The residue was re-dissolved to make a 0.20M solution in 10:1 acetone:water and stirred for 45 min. The mixture was concentrated to give the desired product as a dark red solid. ES/MS m/z 292 (MH⁺).

### Step 2: 2-Amino-5-bromo-6-chloro-3-methoxybenzoic acid

5-Bromo-4-chloro-7-methoxyisatin (1.0 eq) was suspended in 1.0N aqueous sodium hydroxide (10 eq). A 30% solution of hydrogen peroxide in water (4.0 eq) was added, and the reaction was stirred for 20 min at ambient temperature. The mixture was cooled to 0° C. Glacial acetic acid (10 eq) and 3.0 N aqueous hydrochloric acid (10 eq) were added. The solid was collected by vacuum filtration and dried in a vacuum desiccator to give the desired product. ES/MS m/z 282 (MH⁺).

### Step 3: 2-Amino-5-bromo-6-chloro-3-methoxybenzylalcohol

Analogous to Example 9, step 1 using 2-amino-5-bromo-6-chloro-3-methoxybenzoic acid in place of 2-amino-5-bromobenzoic acid. ES/MS m/z 268 (MH⁺).

### Step 4: 2-Amino-5-bromo-6-chloro-3-methoxybenzaldehyde

Analogous to Example 9 step 2 using 2-amino-5-bromo-6-chloro-3-methoxybenzyl alcohol in place of 2-amino-5-bromobenzyl alcohol. ES/MS m/z 266 (min

### Step 5: 6-Bromo-5-chloro-2-hydroxy-8-methoxyquinazoline

Analogous to Example 9, step 3 using 2-amino-5-bromo-6-chloro-3-methoxybenzaldehyde in place of 2-amino-5-bromobenzaldehyde. ES/MS m/z 291 (MH⁺).

### Step 6: 6-Bromo-2,5-dichloro-8-methoxyquinazoline

Analogous to Example 9, step 4 using 6-bromo-5-chloro-2-hydroxy-8-methoxyquinazoline in place of 6-bromo-2-hydroxyquinazoline. ES/MS m/z 309 (MH⁺).

### Step 7: N-(3-(6-Bromo-5-chloro-8-methoxyquinazolin-2-ylamino)-5-((dimethylamino)-methyl)phenyl)acetamide

To a 0.25M solution of 6-bromo-2,5-dichloro-8-methoxyquinazoline in 2-propanol was added N-(3-amino-5-((dimethylamino)methyl)phenyl)acetamide (1.0 eq) and 4.0M HCl in dioxane (1.2 eq). The reaction was stirred at 70° C. for 14 h. The mixture was then concentrated and the resulting residue was used without further purification. Alternatively the crude material was purified by reverse-phase HPLC and lyophilized to yield the desired product as its trifluoroacetic acid salt. ES/MS m/z 480 (MH^{+).}

### Example 15

### (Table 4): 4-(8-Bromo-6-fluoroquinazolin-2-ylamino)benzenesulfonamide

The subject compound was prepared according to the general Scheme 15, below:

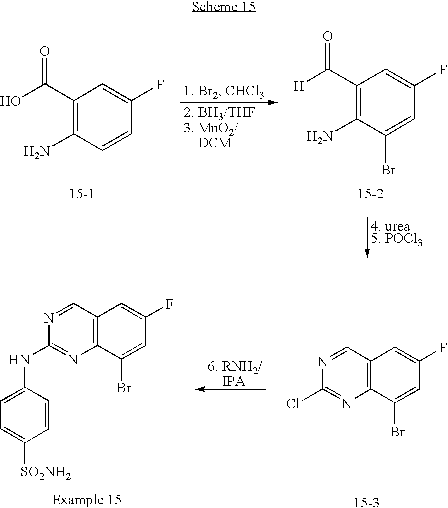

### Step 1

To 2-amino-5-fluorobenzoic acid 15-1, (5 g, 32.2 mmol) in chloroform (90 mL) was added bromine (1.82 mL, 35.4 mmol) in chloroform (10 mL) solution dropwise via an additional funnel. The mixture was stirred at RT for 16 hrs. and LC/MS showed about 50% conversion of the starting material. Additional bromine (1.8 mL) was added to the reaction and continued stirring for another 24 hrs. The resulting white precipitate was collected by filtration, washed thoroughly with DCM and air-dried to give 2-amino-3-bromo-5-fluorobenzoic acid, as its HBr salt. ES/MS m/z 234/236 (MH⁺).

### Step 2: (2-Amino-3-bromo-5-fluorophenyl)methanol

To a 0.5M suspension of 2-amino-3-bromo-5-fluorobenzoic acid in THF in an ice bath was slowly added borane (1.0M/THF, 3 eq). The reaction mixture was stirred at ambient temperature for 24 h. The mixture was recooled to 0° C. and quenched with MeOH and concentrated to remove solvent. The residue was taken into ethyl acetate and organic phase was washed with water, saturated sodium bicarbonate, brine, dried over sodium sulfate and concentrated to give yellow solid in 90% yield. ES/MS m/z 220/222

### Step 3: 2-Amino-3-bromo-5-fluorobenzaldehyde, 15-2

Manganese (IV) oxide (5 eq) was added to a 0.2M solution of (2-amino-3-bromo-5-fluorophenyl)methanol in DCM. The resulting suspension was stirred at ambient temperature under Argon for 12 h. The reaction mixture was filtered through diatomaceous earth and the filter cake was washed with DCM. The combined filtrate was concentrated to give brown color solid. ES/MS m/z 218/220 (MH⁺).

### Step 4: 8-Bromo-6-fluoroquinazolin-2-ol

Solid 8-bromo-6-fluoroquinazolin-2-ol (1 eq) and urea (14 eq) were thoroughly mixed together in a round bottom flask. The mixture was heated to 180° C. in an oil bath for 2.5 h. The reaction mixture was cooled to ambient temperature and water was added to the flask. Filtration gave yellow color solid, which was rinsed with ether and air dried. Yield: 62%. ES/MS m/z 243/245 (MH⁺).

### Step 5: 8-Bromo-2-chloro-6-fluoroquinazoline, 15-3

A 0.5M suspension of 8-bromo-6-fluoroquinazolin-2-ol in phosphorus oxychloride was heated to 110° C. in an oil bath. The suspension was turned to a brown color solution in 20 min. LCMS data showed that the reaction was complete after 1 h. The phosphorus oxychloride was removed by concentration. The residue was mixed with ice water, and adjusted pH to 7 by adding sodium bicarbonate. Reaction mixture was extracted with ethyl acetate. Combined organic phase was washed with water, brine, dried over sodium sulfate and concentrated to give desired product in 89% yield. ES/MS m/z 261/263 (MH⁺).

### Step 6: 4-(8-Bromo-6-fluoroquinazolin-2-ylamino)benzensulfonamide

To a 0.4M suspension of 8-bromo-2-chloro-6-fluoroquinazoline, 15-3 in isopropanol was added 4-aminobenzensulfonamide (1 eq). The reaction mixture was heated to 120° C. in an oil bath for 2 days. LCMS showed that reaction was complete under the condition. Ethyl acetate was added to the reaction flask and the suspension was stirred at ambient temperature for 30 min and was filtered. Filter cake was rinsed with hexane and dried in vacuum to give product in 81% yield. ES/MS m/z 397/399 (MH⁺).

### Example 16

### (Table 4): N-(3-(6-Bromoquinazolin-2-ylamino)-5-(1-methyl-6-oxo-1,6-dihydropyridin-3-yl)phenyl)acetamide

The subject compound was prepared according to the general Scheme 16, below:

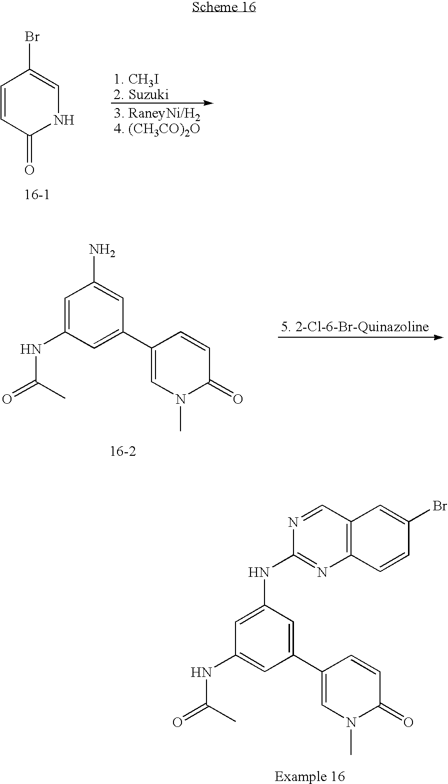

### Step 1: 5-Bromo-1-methylpyridin-2(1H)-one

To a 0.3M suspension of 5-bromo-2(1H)-pyridone in THF in an ice bath was added sodium hydride (2.0 eq). After stirring at 0° C. for 5 min, iodomethane (4.0 eq) was added. The reaction mixture was stirred at ambient temperature for 15 h. Solvent was removed under reduced pressure. The residue was diluted with ethyl acetate and was washed with water, brine, dried over sodium sulfate and concentrated. The crude compound was triturated with hexane and filtered off to collect desired product in 72% yield. ES/MS m/z 188/190 (MH⁺).

### Step 2: 2-(3,5-Dinitrophenyl)-4,4,5,5-tetramethyl-1,3,2-dioxaborolane

To a 0.2M solution of 1-iodo-3,5-dinitrobenzene in dioxane were added bis(pinacolate)diboron (1.5 eq), Pd(dppf)₂Cl₂CH₂Cl₂ (0.2 eq) and flame dried potassium carbonate (2.0 eq). The mixture was purged with Argon for 10 min and was heated to 120° C. for in an oil bath 12 h. The reaction mixture was filtered through diatomaceous earth, and the filter cake was rinsed with dioxane. The combined filtrate was concentrated to provide a residue. The crude residue was purified by Biotage using 20% ethyl acetate in hexane, to give desired product. The structure was confirmed by 1H NMR spectrum.

### Step 3: 5-(3,5-Dinitrophenyl)-1-methylpyridin-2(1H)-one

A 0.2 M mixture of 5-bromo-1-methylpyridin-2(1H)-one (1.0 eq), dinitrophenyl)-4,4,5,5-tetramethyl-1,3,2-dioxaborolane (3.0 eq), Pd(dppf)₂Cl₂CH₂Cl₂ (0.1 eq), 2.0M potassium carbonate (1 eq) in DME was microwave at 120° C. for 15 min.

The reaction mixture was diluted with ethyl acetate, and was washed with water, brine, dried and concentrated. The residue was purified by Biotage using 5% MeOH in dichloromethane (DCM). Product was a brown color solid in 38% yield. ES/MS m/z 276 (MH⁺).

### Step 4: N-(3-Amino-5-(1-methyl-6-oxo-1,6-dihydropyridin-3-yl)phenyl)acetamide

Acetic anhydride (1.0 eq) was added to a 0.06M solution of 5-(3,5-diaminophenyl)-1-methylpyridin-2(1H)-one (1.0 eq), TEA (1.2 eq) in THF in an ice bath. The reaction was monitored by LCMS and was complete in 1 h. Solvent was removed under reduced pressure and residue was purified by RP HPLC. Lyophilization gave product as TFA salt. ES/MS m/z 258 (MH⁺).

### Step 5: N-(3-(6-Bromoquinazolin-2-ylamino)-5-(1-methyl-6-oxo-1,6-dihydropyridin-3-yl)phenyl)acetamide

A 0.08 M suspension of 6-bromo-2-chloroquinazoline (1.0 eq), N-(3-amino-5-(1-methyl-6-oxo-1,6-dihydropyridin-3-yl)phenyl)acetamide (1.0 eq) in isopropanol was heated to 120° C. in an oil bath for 15 h. LCMS showed that conversion was complete under the condition. Solvent was removed under reduced pressure and residue was purified by RP HPLC to give desired product as TFA salt. ES/MS m/z 464/466 (MH⁺).

### Example 17

### (Table 4): N-(3-(6-Ethynylquinazolin-2-ylamino)-5-(1-methyl-6-oxo-1,6-dihydropyridin-3-yl)phenyl)acetamide

The subject compound was prepared according to the general Scheme 17, below:

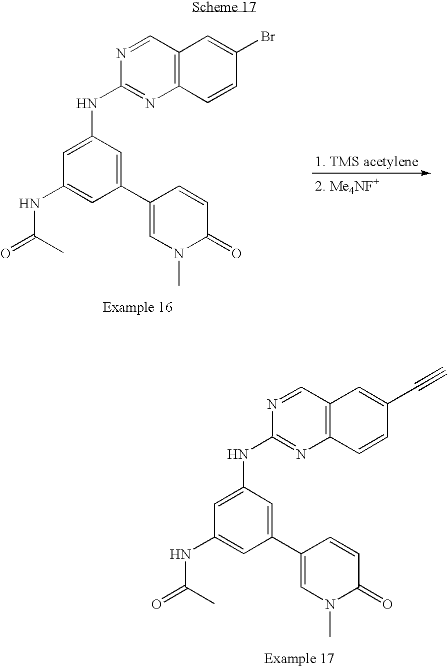

To a 0.04 M mixture of N-(3-(6-bromoquinazolin-2-ylamino)-5-(1-methyl-6-oxo-1,6-dihydropyridin-3-yl)phenyl)acetamide (from Example 16)(0.08 mmol), TEA (0.4 mL), Pd(dppf)₂Cl₂CH₂Cl₂ (0.1 eq), copper(I) iodide (0.1 eq), in DMF was added trimethylsilylacetylene (10 eq). The suspension was microwaved at 120° C. for 20 min. The reaction mixture was diluted with ethyl acetate and was washed with water, brine, dried and concentrated. The oil residue was treated with tetramethylammonium fluoride (1.0 eq) in THF/MeOH (1:1, 0.02M) at ambient temperature for 1 h. Solvent was removed under reduced pressure, and the residue was diluted with ethyl acetate. The organic phase was washed with water, brine, dried and concentrated. The crude product was purified by R^{P} HPLC. Lyophilization gave the desired product. ES/MS m/z 410 (MH⁺).

### Example 18

### (Table 4): Methyl 2-(4-sulfamoylphenylamino)quinazolin-6-carboxylate

The subject compound was prepared according to the general Scheme 18, below:

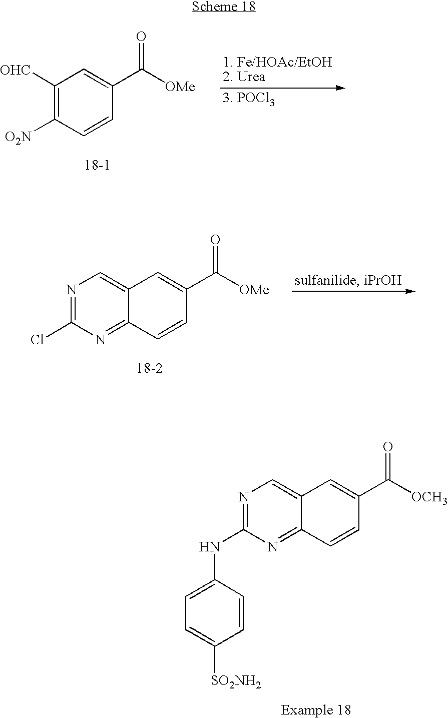

### Step 1: Methyl 3-formyl-4-aminobenzoate, 18-1

To a 1:1 mixture of ethanol and acetic acid was added methyl-3-formyl-4-nitrobenzoate (1 eq) and Fe dust (3 eq) was added in portions. The reduction was complete in 1 h. The reaction mixture was filtered and then concentrated and partitioned between ethyl acetate and water. The organic layer was washed with saturated sodium bicarbonate and dried and concentrated to give methyl 3-formyl-4-aminobenzoate in 85% yield. ES/MS m/z 180 (MH⁺).

### Step 2: Methyl 2-hydroxyquinazoin-6-carboxylate

To methyl 3-formyl-4-aminobenzoate, 18-1 (1 eq) was added urea (5 eq) and the mixture was heated to 145° C. for 16 h. To the crude was added water and the precipitated solid was filtered to give methyl 2-hydroxyquinazoin-6-carboxylate in quantitative yield. ES/MS m/z 205 (MH⁺).

### Step 3: Methyl 2-chloroquinazoin-6-carboxylate, 18-2

To 2-hydroxyquinazoin-7-carboxylate was added POCL₃ and the mixture was added heated to 100° C. for 20 min when the reaction went to completion. To the reaction mixture was added ice and water and the precipitated solid was filtered and dried on the high vacuum overnight to give methyl 2-chloroquinazoin-7-carboxylate in 60% yield. ES/MS m/z 223 (MH⁺).

### Step 4: Methyl 2-(4-sulfamoylphenylamino)quinazolin-6-carboxylate

To methyl 2-chloroquinazoin-6-carboxylate (1 eq) was added sulfanilamide (1 eq) and isopropanol and the mixture was heated to 90° C. for 2 h. The reaction went to completion. The reaction mixture was cooled to RT and filtered to give methyl 2-(4-sulfamoylphenyl amino)quinazolin-7-carboxylate in quantitative yield. ES/MS m/z 359 (MH⁺).

### Example 19

### (Table 4): Methyl 2-(4-sulfamoylphenylamino)quinazolin-6-carboxylic acid

The subject compound was prepared according to the general Scheme 19, below:

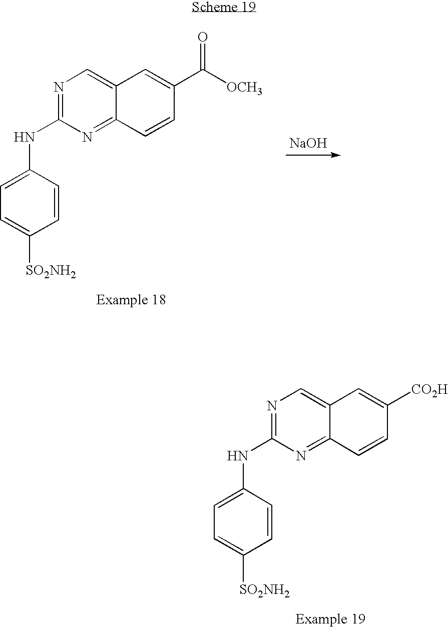

To methyl-2-(4-sulfamoylphenyl amino)quinazolin-7-carboxylate (the compound of Example 18) was added 2N sodium hydroxide (4 eq) and MeOH and the resulting mixture was heated to 80° C. for 10 min. The saponification went to completion. The reaction mixture was concentrated and 1N HCl was added to precipitate methyl 2-(4-sulfamoylphenylamino)quinazolin-7-carboxylic acid as the HCl salt in quantitative yield. ES/MS m/z 344 (MH⁺).

### Example 20

### (Table 4): 4-(6-(4-Methylpiperazine-1-carbonyl)quinazolin-2-ylamino-benzene sulfonamide

The subject compound was prepared according to the general Scheme 20, below:

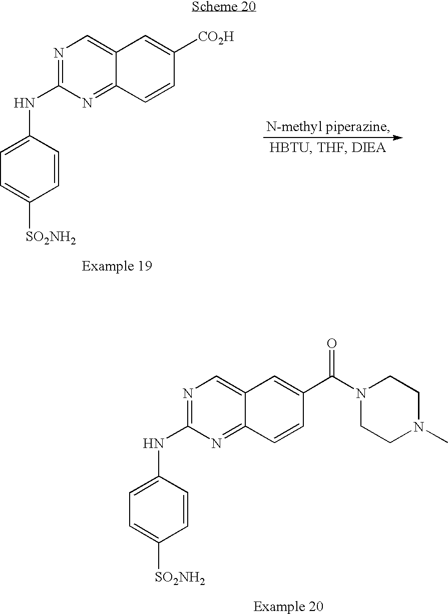

To 2-(4-sulfamoylphenylamino)quinazolin-6-carboxylic acid (from example 19)(1 eq) was added N-methylpiperazine, THF and diisopropylethylamine (DIEA) (4 eq) and HBTU (2 eq) and the mixture was stirred at RT overnight. The coupling went to completion and the mixture was concentrated and partitioned between ethyl acetate and water. The organic layers were concentrated and purified on the prep HPLC to give 4-(6-(4-methylpiperazine-1-carbonyl)quinazolin-2-ylamino-benzene sulfonamide in 50% yield. ES/MS m/z 427 (MH⁺).

### Example 21

### (Table 4): 4-(6-(1-Isobutyl-1H-pyrazol-4-yl)quinazolin-2-ylamino)benzenesulfonamide

The subject compound was prepared according to the general Scheme 21, below:

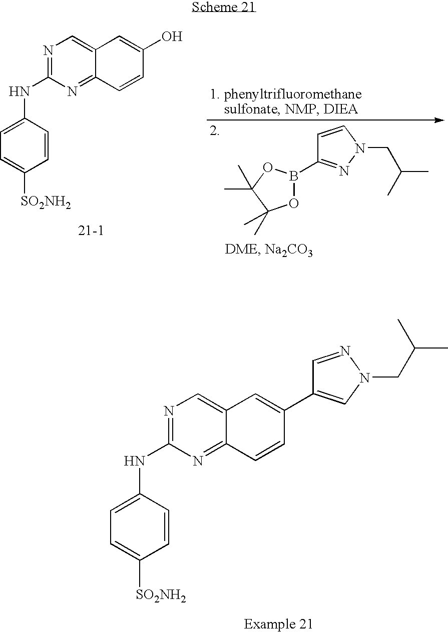

### Step 1: 2-(4-Sulfamoylphenylamino) quinazolin-6-yltrifluoromethane sulfonate

To a solution of 4-(6-hydroxyquinazolin-2-ylamino) benzenesulfonamide, 21-1 (1 eq) in NMP was added phenyltrifluoromethanesulfonate (1.2 eq) and DIEA (2.5 eq) and the reaction mixture was stirred over night at ambient temperature. The reaction mixture was then partitioned between ethyl acetate and water. The organic layers were washed with saturated sodium chloride and dried and concentrated. To the crude was added DCM and few drops of MeOH. The white solid hence formed was filtered to give 2-(4-sulfamoylphenylamino)quinazolin-6-yltrifluoromethane sulfonate in 80% yield. ES/MS m/z 447 (MH⁺).

### Step 2: 4-(6-(1-Isobutyl-1H-pyrazol-4-yl)quinazolin-2-ylamino)benzenesulfonamide

To a solution of 2-(4-sulfamoylphenylamino)quinazolin-7-yltrifluoromethane sulfonate (1 eq) in DME was added 2M sodium carbonate solution and 1-isobutyl-3-(4,4,5,5-tetramethyl-1,3,2-dioxaborolan-2-yl)-1H-pyrazole (3 eq) and Pd(dppf)₂Cl₂.CH₂Cl₂ (0.05 eq) and the mixture was micro waved for 10 min at 120° C. The reaction mixture was then partitioned between ethyl acetate and water. The organic layer was concentrated to yield 4-(6-(1-isobutyl-1H-pyrazol-4-yl)quinazolin-2-ylamino)benzenesulfonamide. ES/MS m/z 423 (MH⁺).

### Example 22

### (Table 4): (4-(5-Chloro-6-ethynylquinazolin-2-ylamino)phenyl)(morpholino) methanone

The subject compound was prepared according to the general Scheme 22 below:

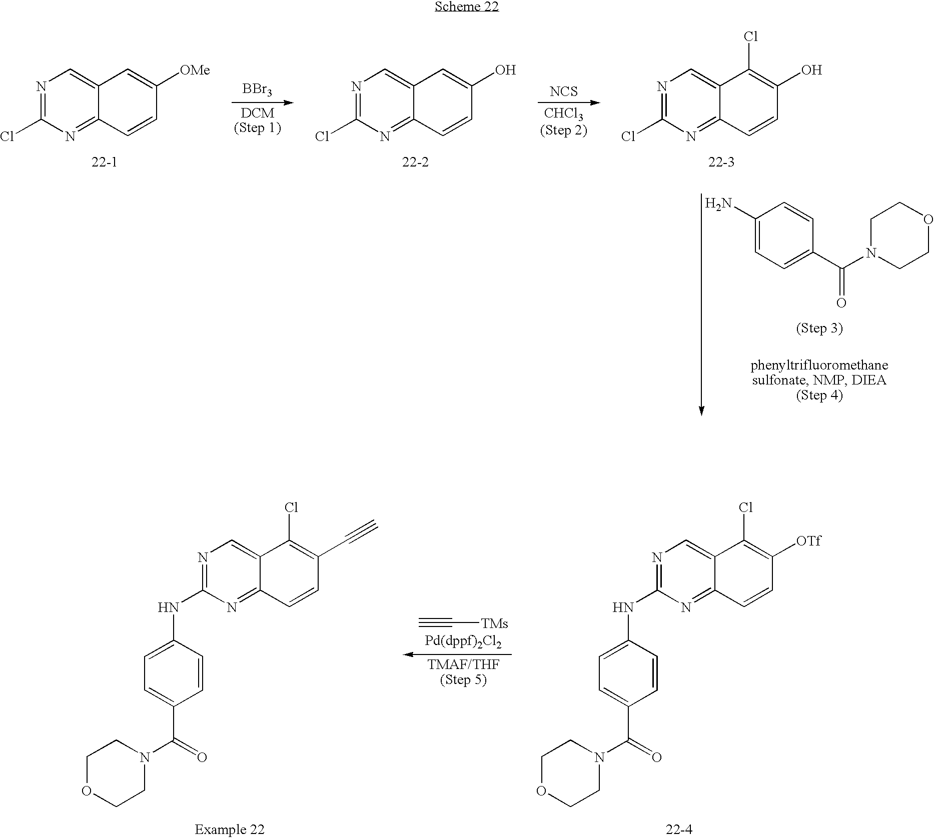

### Step 1: 2-Chloroquinazolin-6-ol, 22-2

To 2-chloro-6-methoxy quinazoline, 22-1 in DCM was added boron tribromide (2 eq) and the mixture was heated at 40° C. for 16 h. The deprotection of the methyl ether goes to completion by LC/MS. The mixture was concentrated and the solid was filtered and washed with ice/water and the solid was dried on high vacuum to give 2-chloroquinazolin-6-ol. ES/MS m/z 181 (MH⁺).

### Step 2: 2,5-Dichloroquinazolin-6-ol, 22-3

To 2-chloroquinazolin-6-ol, 22-2 (1 eq) in chloroform was added N-chlorosuccinimide (1 eq) and the mixture was heated to 40° C. for 16 h. The reaction goes to completion to give 2,5-dichloroquinazolin-6-ol that was observed by LC/MS and the structure was confirmed by ¹HNMR. The mixture was concentrated and purified on silica gel to give the product. ES/MS m/z 215 (MH⁺).

### Step 3: 2-Chloro5-bromoquinazolin-6-ol

To 2-chloroquinazolin-6-ol, 22-2 (1 eq) in chloroform was added N-bromosuccinimide (1 eq) and the mixture was stirred at ambient temperature for 1 h. The reaction goes to completion to give 2-chloro5-bromoquinazolin-6-ol that was observed by LC/MS and the structure was confirmed by ¹HNMR. The mixture was concentrated and passed through a silica gel plug to give the product in quantitative yield. ES/MS m/z 260 (MH⁺).

### Step 4: (4-(5-Chloro-6-hydroxyquinazolin-2-ylamino)phenylymorpholino)methanone

To 2,5-dichloroquinazolin-6-ol, 22-3 (1 eq) in isopropanol was added (4-amino-phenyl)(morpholino)methanone (1 eq) and the reaction mixture was heated to 90° C. for 1 h. The reaction went to completion by LC/MS. The mixture was then concentrated and used without further purification. ES/MS m/z 386 (MH⁴).

### Step 5: 5-Chloro-2-(4-(morpholine-4-carbonyl)phenylamino)quinazolin-6-yl trifluoro-methanesulfonates, 22-4

To a solution of (4-(5-chloro-6-hydroxyquinazolin-2-ylamino)phenyl)(morpholino) methanone (1 eq) in NMP was added phenyltrifluoromethanesulfonate (1.2 eq) and DIEA (2.5 eq) and the reaction mixture was stirred over night at ambient temperature. The reaction mixture was then partitioned between ethyl acetate and water. The organic layers were washed with saturated sodium chloride and dried and concentrated. To the crude was added methylene chloride and few drops of MeOH. The white solid hence formed was filtered to give 5-chloro-2-(4-(morpholine-4-carbonyl)phenylamino) quinazolin-6-yl trifluoromethanesulfonate in 80% yield. ES/MS m/z 517 (MH⁺).

### Step 6: (4-(5-Chloro-6-ethynylquinazolin-2-ylamino)phenyl)(morpholino) methanone

To 5-chloro-2-(4-(morpholine-4-carbonyl)phenylamino) quinazolin-6-yl trifluoromethanesulfonate, 22-4 (1 eq) in 4:1 DMF and TEA was added TMS acetylene (4 eq) and copper iodide (0.2 eq) and Pd(dppf)₂Cl₂.CH₂Cl₂ (0.2 eq) and the mixture was microwaved for 10 min at 120° C. The reaction mixture was then partitioned between ethyl acetate and water. The organic layer was concentrated to yield (4-(5-chloro-6-((trimethylsilyl)ethynyl)quinazolin-2-ylamino)phenyl)(morpholino)methanone. ES/MS m/z 465 (MH⁺). To the crude was added THF and tetramethylammonium fluoride (1 eq) and the mixture was stirred at ambient temperature for 1 h. It was then partitioned between ethyl acetate and water and the organic layer was concentrated and purified on prep HPLC to give (4-(5-chloro-6-ethynylquinazolin-2-ylamino)phenyl)(morpholino) methanone. ES/MS m/z 393 (MH⁺).

### Example 23

### (Table 4): 6-Bromo-5-fluoro-N-(4-morpholinophenyl)quinazolin-2-amine

The subject compound was prepared according to the general Scheme 23 below:

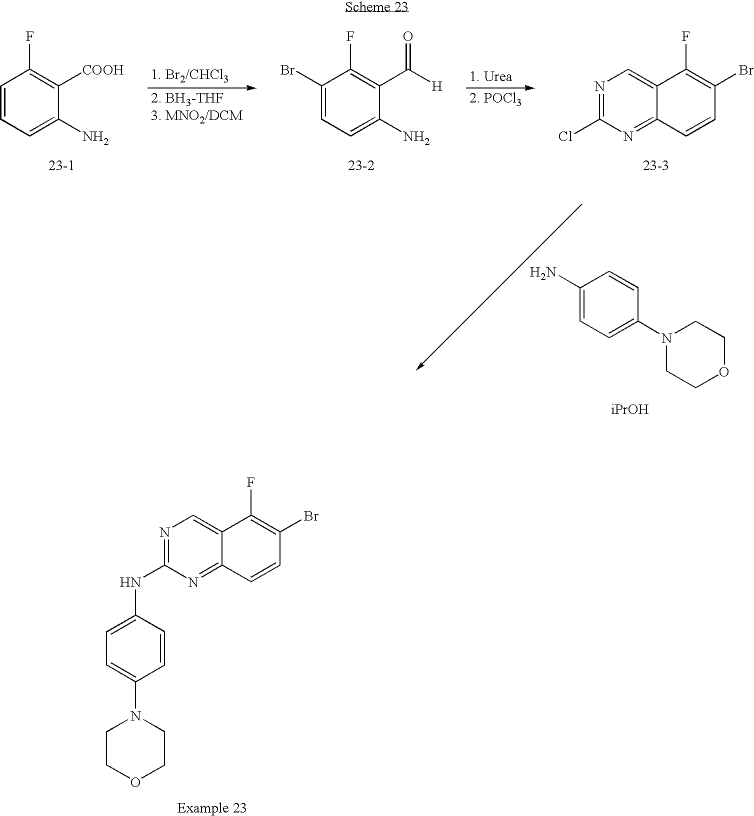

### Step 1: 6-Amino-3-bromo-2-fluorobenzoic acid

To 2-amino-6-fluorobenzoic acid, 23-1 (1 eq) in chloroform at 0° C. was added bromine (1.2 eq) drop-wise and the mixture was stirred at ambient temperature for 16 h. Formation of 6-amino-3-bromo-2-fluorobenzoic acid (47%) was observed by LC/MS along with 2-amino-3-bromo-6-fluorobenzoic acid (22%) and 20% of 2-amino-3,5-dibromo-6-fluorobenzoic acid and 9% starting material remained. The structures of the isomers were confirmed by ¹HNMR. The reaction mixture was concentrated and filtered and the solid was washed with chloroform to give an off white solid. The crude mixture was carried on to the next step without purification. ES/MS m/z 235 (MH⁺).

### Step 2: (6-Amino-3-bromo-2-fluorophenyl)methanol

To the crude mixture from step 1 in THF at 0° C. in a flame dry flask was added borane-THF complex (4 eq) dropwise. The mixture was brought to ambient temperature and was stirred for 16 h. The formation of (6-amino-3-bromo-2-fluorophenyl)methanol was observed by LC/MS. The reaction mixture was concentrated and to the crude was partitioned between water and ethyl acetate. The organic layer was washed with brine and dried (Na₂SO₄). The crude yellow oil was purified on silica gel and the formation of (6-amino-3-bromo-2-fluorophenyl)methanol was confirmed by ¹HNMR. ES/MS m/z 218 (MH⁺).

### Step 3: 6-Amino-3-bromo-2-fluorobenzaldehyde, 23-2

To (6-amino-3-bromo-2-fluorophenyl)methanol (1 eq) in methylene chloride was added manganese dioxide (8 eq) and the mixture was stirred at ambient temperature for 16 h. Formation of 6-amino-3-bromo-2-fluorobenzaldehyde was confirmed by LC/MS. The mixture was then filtered and the filtrate was concentrated to give 6-amino-3-bromo-2-fluorobenzaldehyde. ES/MS m/z 218 (MH⁺).

### Step 4: 6-Bromo-5-fluoroquinazolin-2-ol

To methyl 6-amino-3-bromo-2-fluorobenzaldehyde, 23-2 (1 eq) was added urea (8 eq) and the mixture was heated to 180° C. for 1 h. To the crude was added water and the precipitated solid was filtered and dried under vacuum to give 6-bromo-5-fluoroquinazolin-2-ol. ES/MS m/z 242 (MH⁺).

### Step 5: 6-Bromo-2-chloro-5-fluoroquinazoline, 23-3

To 6-bromo-5-fluoroquinazolin-2-ol was added POCl₃ and the mixture was added heated to 100° C. for 2 h when the reaction went to completion. To the reaction mixture was added ice and water and the precipitated solid was filtered and dried on the high vacuum overnight to give 6-bromo-2-chloro-5-fluoroquinazoline. ES/MS m/z 260 (MH⁺).

### Step 6: 6-Bromo-5-fluoro-N-(4-morpholinophenyl)quinazolin-2-amine

To a solution of 6-bromo-2-chloro-5-fluoroquinazoline, 23-3 (1 eq) in isopropanol was added 4-morpholinoaniline (1 eq) and the mixture was heated to 90° C. for 1 h in a sealed tube. The SNAR went to completion by LC/MS and purification on prep HPLC yielded 6-bromo-5-fluoro-N-(4-morpholinophenyl)quinazolin-2-amine in quantitative yield. ES/MS m/z 403 (MH⁺).

### Example 24

### (Table 4): 6-Ethynyl-5-fluoro-N-(4-morpholinophenyl)quinazolin-2-amine

The subject compound was prepared according to the general Scheme 24 below:

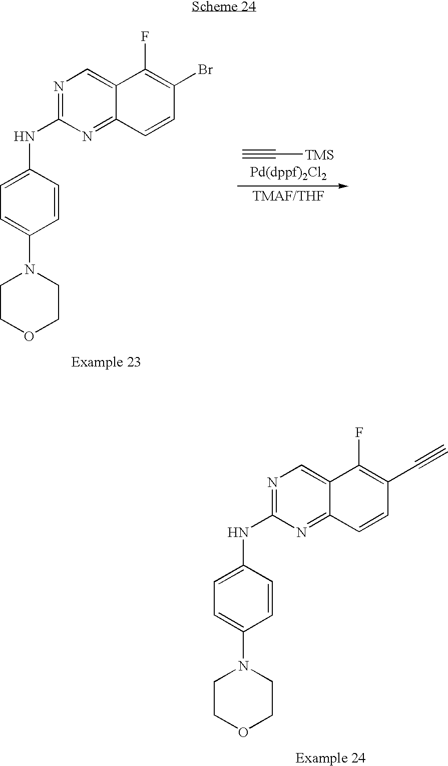

To 6-bromo-5-fluoro-N-(4-morpholinophenyl)quinazolin-2-amine (the compound of Example 20)(1 eq) in 4:1 DMF and TEA was added TMS acetylene (4 eq) and copper iodide (0.2 eq) and Pd(dppf)₂Cl₂.CH₂Cl₂ (0.2 eq) and the mixture was microwaved for 10 min at 120° C. The reaction mixture was then partitioned between ethyl acetate and water. The organic layer was concentrated to yield 6-ethynyl-5-fluoro-N-(4-morpholinophenyl)-quinazolin-2-amine ES/MS m/z 420 (MH⁺). To the crude was added THF and tetramethyl ammonium fluoride (1 eq) and the mixture was stirred at ambient temperature for 1 h. It was then partitioned between ethyl acetate and water and the organic layer was concentrated and the resulting residue was purified on prep HPLC to give (4-(5-chloro-6-ethynylquinazolin-2-ylamino)phenyl)(morpholino) methanone. ES/MS m/z 349 (MH⁺).

### Example 25

### (Table 4): N-(3-(6-Bromo-5-fluoroquinazolin-2-ylamino)-5-(morpholinomethyl)phenyl)acetamide

The subject compound was prepared according to the general Scheme 25 below:

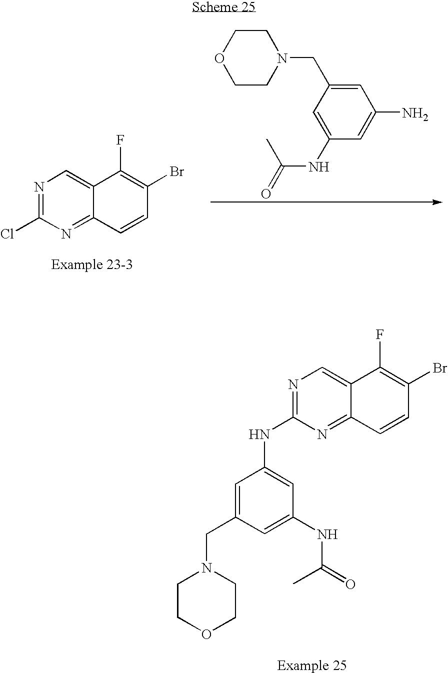

The compound N-(3-(6-bromo-5-fluoroquinazolin-2-ylamino)-5-(morpholino-methyl)phenyl)acetamide, was prepared by a procedure analogous to Example 23.

### Example 26

### (Table 4): N-(3-(5-Fluoro-6-(thiazol-2-yl)quinazolin-2-ylamino)-5-(morpholinomethyl)phenyl)acetamide

The subject compound was prepared according to the general Scheme 26 below:

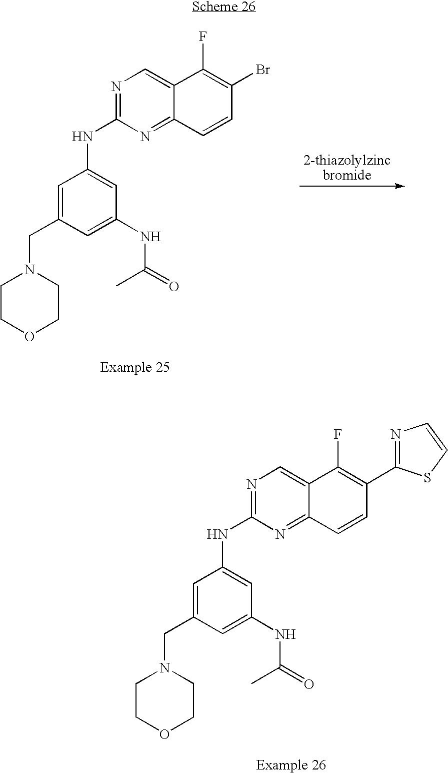

To N-(3-(6-bromo-5-fluoroquinazolin-2-ylamino)-5-(morpholinomethyl)phenyl)acetamide (from Example 25) (1 eq) was added 2-thiazolylzinc bromide solution in THF an the mixture a microwaved at 120° C. for 10 min. Formation of N-(3-(5-fluoro-6-(thiazol-2-yl)quinazolin-2-ylamino)-5-(morpholinomethyl)phenylacetamide was confirmed by LC/MS. It was then concentrated and purified on prep HPLC to give the product. ES/MS m/z 479 (MH⁺).

### Example 27

### (Table 4): 4-(6-(Thiazol-2-yl)quinazolin-2-ylamino)benzenesulfonamide

The subject compound was prepared according to the general Scheme 27 below:

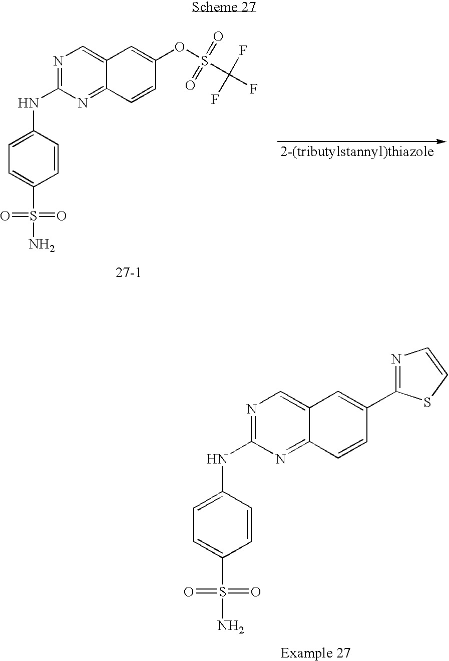

To 2-(4-sulfamoylphenylamino)quinazolin-6-yltrifluoromethane sulfonate (prepared by following Example 18 step 1) (1 eq) in DMF was added 2-(tributylstannyl)thiazole (3 eq) and TEA (6 eq). The mixture was microwaved at 120° C. for 10 min. The LC/MS showed formation of the 4-(6-(thiazol-2-yl)quinazolin-2-ylamino)benzenesulfonamide. The crude mixture after work up was then purified on prep HPLC to give 4-(6-(thiazol-2-yl)quinazolin-2-ylamino) benzene sulfonamide. ES/MS m/z 384 (MH⁴).

### Example 28

### (Table 4): 5-Chloro-N-(4-morpholinophenyl)-6-(thiazole-2-yl) quinazolin-2-amine

The subject compound was prepared according to the general Scheme 28 below:

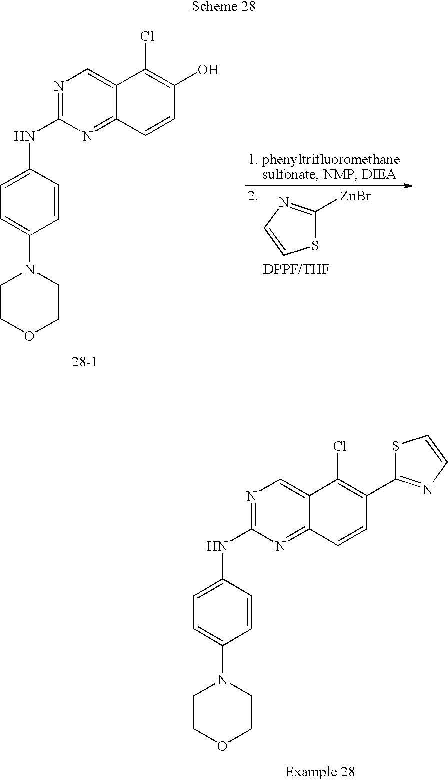

### Step 1: 5-Chloro-2-(4-(morpholinophenylamino) quinazolin-6-yltrifluoromethane sulfonate

See, Example 22 for the synthesis. ES/MS m/z 489.1 (MH⁺).

### Step 2: 5-Chloro-N-(4-morpholinophenyl)-6-(thiazole-2-yl) quinazolin-2-amine

A mixture of 5-chloro-2-(4-(morpholinophenylamino) quinazolin-6-yltrifluoro-methane sulfonate (1 eq), 2-thiazolylzincbromide (5 eq, 0.5M soln in THF)) and Pd (dppf)₂Cl₂.CH₂Cl₂ (0.2 eq) in THF was microwaved for 20 min at 120° C. The LC-MS shows formation of two products in 1:1 ratio. The reaction mixture was concentrated and purified by semi-preparative HPLC to provide 5-chloro-N-(4-morpholinophenyl)-6-(thiazole-2-yl) quinazolin-2-amine in 25% yield. ES/MS m/z 424.1 (MH⁺).

The second product of the reaction was identified as: N-(4-morpholinophenyl)-5,6-di(thiazole-2-yl) quinazolin-2-amine. ES/MS m/z 473.0 (MH⁺).

### Example 29

### (Table 4): N-(3-(6-Bromoquinazolin-2-ylamino)-5-(morpholinomethyl)phenyl)acetamide

The subject compound was prepared according to the general Scheme 29 below:

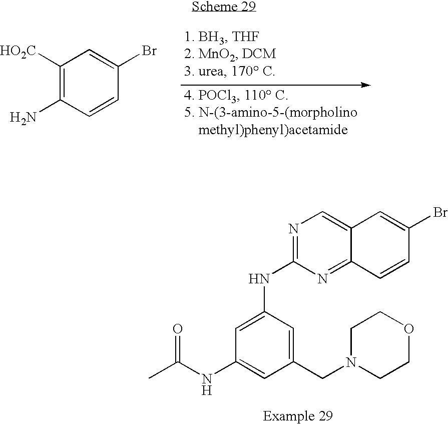

The compound, N-(3-(6-bromoquinazolin-2-ylamino)-5-(morpholinomethyl)phenyl)acetamide, was prepared by a synthesis analogous to that used in Example 9. ES/MS m/z 456.0 (MIT).

### Example 30

### (Table 4): N-(3-(6-(1H-Pyrazol-4-yl) quinazolin-2-ylamino)-5-(morpholinomethyl)phenyl)acetamide

The subject compound was prepared according to the general Scheme 30 below:

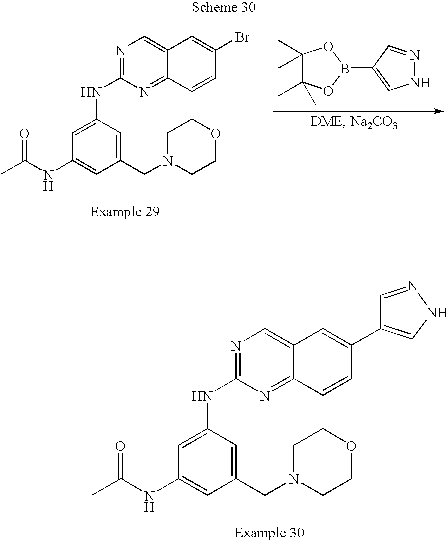

To a solution of N-(3-(6-bromoquinazolin-2-ylamino)-5-(morpholinomethyl)phenyl)acetamide (from Example 29) (1 eq) in DME was added 2M sodium carbonate solution and 4-(4,4,5,5,-tetramethyl-1,3,2-dioxaborolan-2-yl)-1H-pyrazole (3 eq) and Pd (dppf)₂Cl₂.CH₂Cl₂ (0.05 eq) and the mixture was microwaved for 10 min at 120° C. The reaction mixture was then partitioned between ethyl acetate and water. The organic layer was washed with brine, dried, concentrated and purified by semi-preparative HPLC to provide N-(3-(6-(1H-pyrazol-4-yl) quinazolin-2-ylamino)-5-(morpholinomethyl)phenyl)acetamide; 25% yield. ES/MS m/z 444.3 (MH⁺). N-(3-(morpholinomethyl)-5-(quinazolin-2-ylamino) phenyl)acetamide was isolated as a side product. ES/MS m/z 378.2 (MH⁺).

### Example 31

### (Table 4): N-(3-(6-Bromoquinazolin-2-ylamino)-5-iodophenyl)acetamide

The subject compound was prepared according to the general Scheme 31 below:

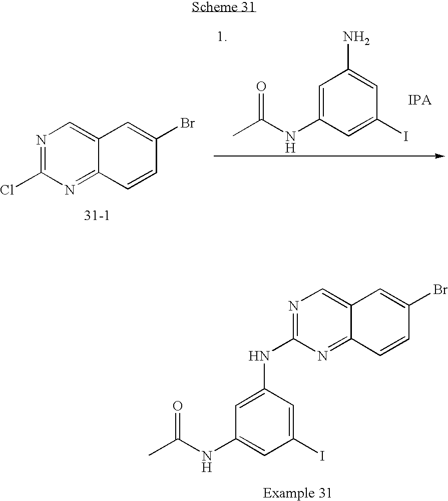

A mixture of 6-bromo-2-chloroquinazoline, 31-1 (1 eq) and N-(3-amino-5-iodo-phenyl)acetamide (1 eq) in 2-propanol was heated at 110° C. overnight. Product was precipitated in the reaction mixture. The precipitate was filtered, washed and dried under vacuum to provide pure product as a yellow solid in 99% yield. ES/MS m/z 482.9 (MH⁺).

### Example 32

### (Table 4): N-(3-(6-Bromoquinazolin-2-ylamino)-5-(pyridine-3-yl)phenyl)acetamide; and

### Example 33

### (Table 4): N-(3-(Pyridin-3-yl)-5-(6-(pyridin-3-yl) quinazolin-2-ylamino) phenyl)acetamide

The subject compounds were prepared according to the general Scheme 32/33, below:

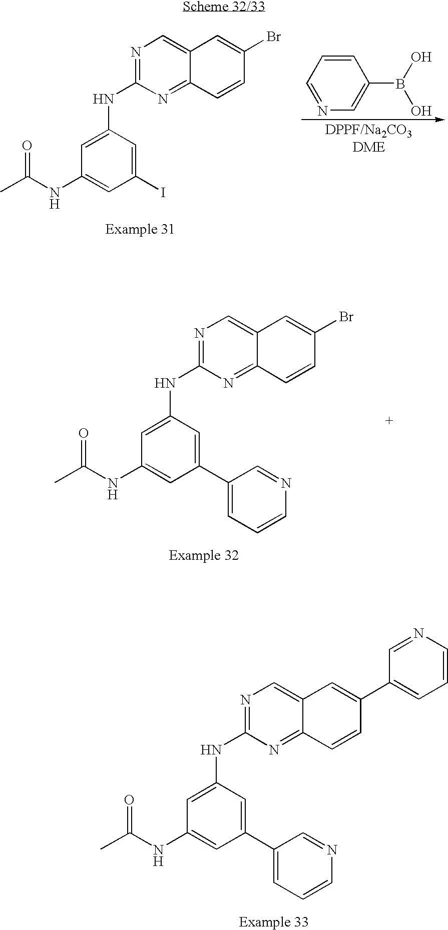

To the compound of Example 26 (1 eq) in DME was added 2M sodium carbonate solution, 3-pyridyl boronic acid (2 eq) and Pd(dppf)₂Cl₂.CH₂Cl₂ (0.05 eq) and the mixture was microwaved for 10 min at 120° C. LC-MS shows formation of two products. The reaction mixture was then partitioned between ethyl acetate and water. The organic layer was washed with brine, dried, concentrated and purified by semi-preparative HPLC to provide N-(3-(6-bromoquinazolin-2-ylamino)-5-(pyridine-3-yl) phenyl)acetamide in 60% yield. ES/MS m/z 434.1 (MH⁴).

The second product of this reaction (Example 33), was identified as: N-(3-(pyridin-3-yl)-5-(6-(pyridin-3-yl) quinazolin-2-ylamino) phenyl)acetamide. ES/MS m/z 433.2 (MH⁺).

### Example 34

### (Table 4): N-(3-(6-Ethynylyquinazolin-2-ylamino)-5-(pyridine-3-yl)phenyl)acetamide

The subject compound was prepared according to the general Scheme 34 below:

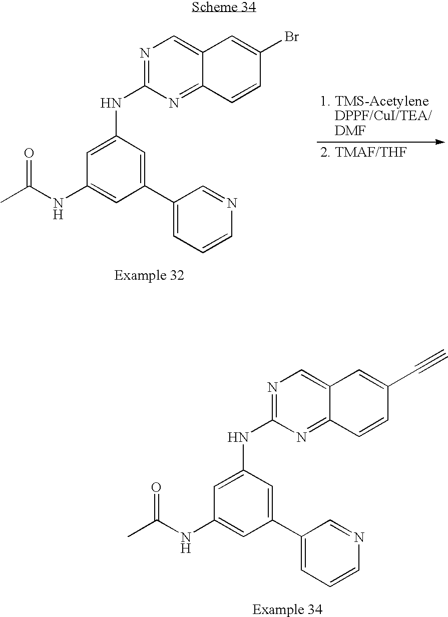

To N-(3-(6-bromoquinazolin-2-ylamino)-5-(pyridine-3-yl)phenyl)acetamide (1 eq) in 4:1 DMF and TEA was added TMS acetylene (4 eq) and copper iodide (0.2 eq) and Pd(dppf)₂Cl₂.CH₂Cl₂(0.2 eq) and the mixture was micro waved for 10 min at 120° C. The reaction mixture was then partitioned between ethyl acetate and water. The organic layer was concentrated to yield N-(3-(pyridine-3-yl)-5-(6-((trimethylsilyl)ethynyl) quinazolin-2-ylamino)phenyl)acetamide. ES/MS m/z 452.1 (MH⁺). To the crude was added THF and tetramethyl ammonium fluoride (1 eq) and the mixture was stirred at ambient temperature for 1 h. It was then partitioned between ethyl acetate and water and the organic layer was concentrated and purified by semi-prep HPLC to provide N-(3-(6-ethynylyquinazolin-2-ylamino)-5-(pyridine-3-yl)phenyl)acetamide. ES/MS m/z 380.1 (MH⁺).

### Example 35

### (Table 4): 4-(6,7-Dimethoxyquinazolin-2-ylamino) benzenesulfonamide

The subject compound was prepared according to the general Scheme 35 below:

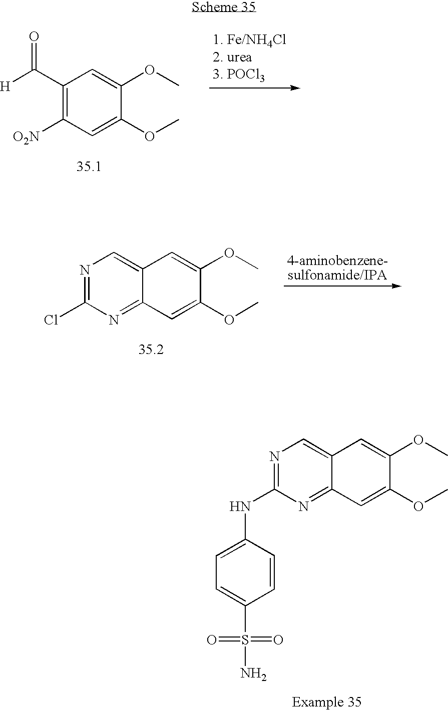

### Step 1: 2-Amino-4,5-dimethoxybenzaldehyde

To a solution of 4,5-dimethoxy-2-nitrobenzaldehyde (1 eq) in ethanol and water (2:1) was added ammonium chloride (10 eq). The solution was heated at 90° C. followed by the addition of iron powder (4 eq) in portions. The reaction mixture was heated at 90° C. for 30 min., cooled, diluted with DCM and filtered through diatomaceous earth. The organic layer was separated from aq layer, washed with brine and dried over sodium sulfate, filtered, concentrated and dried under vacuum to provide the product in 93% yield. ES/MS m/z 182.1 (MH⁴).

### Step 2: 6,7-Dimethoxyquinazolin-2-ol

A mixture of 2-amino-4,5-dimethoxybenzaldehyde (1 eq) (obtained from step 1) and urea (15 equiv.) was heated to 175° C. with vigorous stirring for 2 h. The reaction was cooled to RT and water was added. A solid precipitate formed and was collected by filtration, and air-dried to give 6,7-dimethoxyquinazolin-2-ol as a brown solid in 40% yield. ES/MS m/z 207.0 (MH⁺).

### Step 3: 2-Chloro-6,7-dimethoxyquinazoline, 35-2

The crude 6,7-dimethoxyquinazolin-2-ol was heated in neat phosphorus oxychloride (POCl₃) at 110° C. for 2 h. The resulting mixture was cooled to RT and concentrated in vacuo to nearly dryness. Ice water was added and the pH was adjusted to ˜6 using sodium bicarbonate. Extraction with DCM followed by drying with sodium sulfate and concentration in vacuo yielded 2-chloro-6,7-dimethoxyquinazoline as a brown solid. ES/MS m/z 225.0 (MH⁺).

### Step 4: 4-(6,7-Dimethoxyquinazolin-2-ylamino) benzenesulfonamide

A mixture of 2-chloro-6,7-dimethoxyquinazoline (1 eq) and 4-aminobenzene-sulfonamide (1 eq) in isopropanol was heated at 90° C. for 16 h. The product precipitated in the reaction mixture, and was separated by filtration, washed and dried to provide the pure product as a yellow solid in 87% yield. ES/MS m/z 361.0 (MH⁺).

### Example 47

### (Table 4): 4-(6-Bromo-7-methoxyquinazolin-2-ylamino)benzenesulfonamide

The subject compound was prepared according to the general Scheme 36 below:

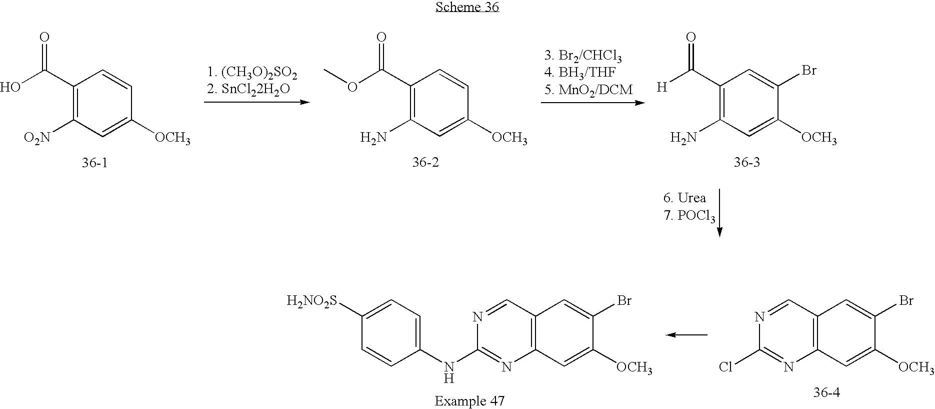

### Step 1: Methyl 4-methoxy-2-nitrobenzoate

To an ice cooled 0.4M solution of 4-methoxy-2-nitrobenzoic acid 36-1, in THF was added TEA (6.0 eq), followed by the addition of dimethyl sulfate (4.0 eq). The resulting mixture was stirred at 0° C. for 1 h, and then at ambient temperature overnight. LCMS showed about 90% conversion to the product. The solvent was removed under reduced pressure. The resulting residue was diluted with ethyl acetate and then washed sequentially with saturated sodium bicarbonate and brine, and then dried over sodium sulfate. Concentration of the dried solution provided the desired product as an off-white solid in 94% yield. The structure of the product was confirmed by proton NMR spectrum.

### Step 2: Methyl 2-amino-4-methoxybenzoate, 36-2

To a 0.4M solution of methyl 4-methoxy-2-nitrobenzoate in DMF in an ice bath was added Tin(II) chloride dihydrate (7.0 eq). The mixture was stirred at ambient temperature overnight, then diluted with ethyl acetate (40 mL ethyl acetate for each mM of the nitro compound). TEA (14 eq) was added, and the resulting white suspension was stirred for 1 h, and was then filtered. The filter cake was rinsed with ethyl acetate. The combined organic phase was washed with water and then brine, and was then dried over sodium sulfate. Concentration of the dried organic phase provided the desired product in 95% yield. ES/MS m/z 182 (MH⁺).

### Step 3: Methyl 2-amino-5-bromo-4-methoxybenzoate

To a 0.06M solution of methyl 2-amino-4-methoxybenzoate, 36-2, in chloroform was added bromine (1.0 eq as a 0.05M solution in chloroform) via an additional funnel. The resulting mixture was stirred at RT for 3 h. LCMS data showed that the bromination reaction had proceeded to about 72% conversion. The resulting white precipitate was collected by filtration, washed thoroughly with DCM and then air-dried to provide methyl 2-amino-5-bromo-4-methoxybenzoate as its HBr salt. Yield: 70%. ES/MS m/z 260/262 (MH⁺).

### Step 4: (2-Amino-5-bromo-4-methoxyphenyl)methanol

To a 0.25M suspension of methyl 2-amino-5-bromo-4-methoxybenzoate in THF in an ice bath was slowly added borane (4.5 eq as a 1.0M THF solution). The reaction mixture was stirred at ambient temperature for 48 h. The mixture was then recooled to 0° C., quenched with MeOH and concentrated to remove the solvent. The resulting residue was dissolved in ethyl acetate and the resulting organic phase was washed with water, and then brine, and then dried over sodium sulfate and concentrated to provide (2-amino-5-bromo-4-methoxyphenyl)methanol, as a brown colored oil. ES/MS m/z 214/216

### Step 5: 2-Amino-5-bromo-4-methoxybenzaldehyde, 36-3

Manganese (IV) oxide (8 eq) was added to a 0.22M solution of (2-amino-5-bromo-4-methoxyphenyl)methanol in DCM. The resulting suspension was stirred at ambient temperature under Argon for 12 h. The reaction mixture was then filtered through diatomaceous earth and the resulting filter cake was washed with DCM. The combined filtrate was concentrated to provide the product as a brown colored solid in 67% yield. ES/MS m/z 230/232 (MH⁺).

### Step 6: 6-Bromo-7-methoxyquinazolin-2-ol

A mixture of 2-amino-5-bromo-4-methoxybenzaldehyde, 36-3, (1 eq) and urea (14 eq) was heated to 180° C. in an oil bath under Argon for 2 h. Water was added after cooling to ambient temperature. The solid was collected by filtration and air dried to give product in 90% yield. ES/MS m/z 255/257 (MH⁺).

### Step 7: 6-Bromo-2-chloro-7-methoxyquinazoline, 36-4

A 0.5M suspension of 6-bromo-7-methoxyquinazolin-2-ol in phosphorus oxychloride was heated to 110° C. in an oil bath for 3 h. The mixture was then cooled to RT. The volatiles were removed under reduced pressure. The resulting residue was triturated with ice water. The resulting solid was collected by filtration and air dried to provide the product in 55% yield. ES/MS m/z 273/275 (MW).

### Step 8: 4-(6-Bromo-7-methoxyquinazolin-2-ylamino)benzenesulfonamide

To a solution of 50 mg of 36-4 in 2-propanol (1 mL) was added sulfanilamide (1.0 eq). The reaction was stirred at 90° C. for 18 hours. The hydrochloride was collected by vacuum filtration and air dried to give a crude material which can be used for further chemical modifications. The pure material was obtained by HPLC purification. ES/MS m/z 409/411 (MW).

### Example 68

### (Table 4): N-(2-(4-Sulfamoylphenylamino)quinazolin-6-yl)acetamide

The subject compound was prepared according to the general Scheme 37 below:

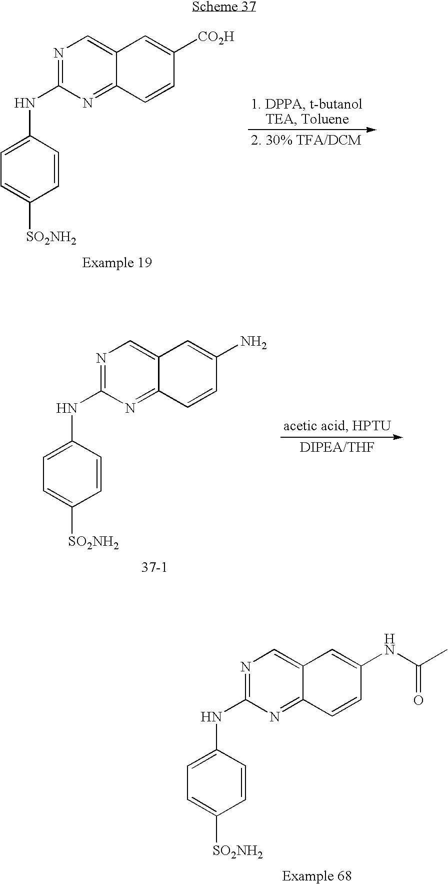

### Step 1: 2-(4-Sulfamoylphenylamino)quinazolin-6-yl tert-butylcarbamate

To 2-(4-sulfamoylphenylamino) quinazoline-6-carboxylic acid (1 eq) (prepared as in Example 19) in toluene was added diphenylphosphorylazide (DPPA) (1.2 eq), tert-butanol (10 eq) and TEA (2 eq). The resulting mixture was heated to 70° C. for 30 min, and was then heated further to 100° C. and maintained at 100° C. overnight. The reaction mixture was then concentrated and the resulting residue was purified by semi-prep HPLC to provide the pure product.

### Step 2: 4-(6-Aminoquinazolin-2-ylamino)benzenesulfonamide, 37-1

A solution of 2-(4-sulfamoylphenylamino)quinazolin-6-yl tert-butylcarbamate in 30% TFA/DCM was stirred at RT for 30 min. The solvent was then evaporated, and the resulting crude residue was purified by semi-prep HPLC to provide the product.

### Step 3: N-(2-(4-Sulfamoylphenylamino)quinazolin-6-yl)acetamide

To a solution of 4-(6-aminoquinazolin-2-ylamino)benzenesulfonamide, 37-1. (1 eq) in THF was added acetic acid (5 eq), HBTU (4 eq) and DIEA (10 eq). The resulting mixture was stirred at RT for 48 h. The reaction does not go to completion. The reaction mixture was diluted with ethyl acetate and the resulting diluted mixture was washed with water, and then brine and then was dried over sodium sulfate. The dried mixture was filtered, and then concentrated. The concentrate was purified by semi-prep HPLC to provide N-(2-(4-sulfamoylphenylamino)quinazolin-6-yl)acetamide.

### Example 135

### (Table 4): N-(3-(6-Bromo-8-fluoroquinazolin-2-ylamino)-5-((dimethylamino)methyl)phenyl)acetamide

The subject compound was prepared according to the general Scheme 38 below:

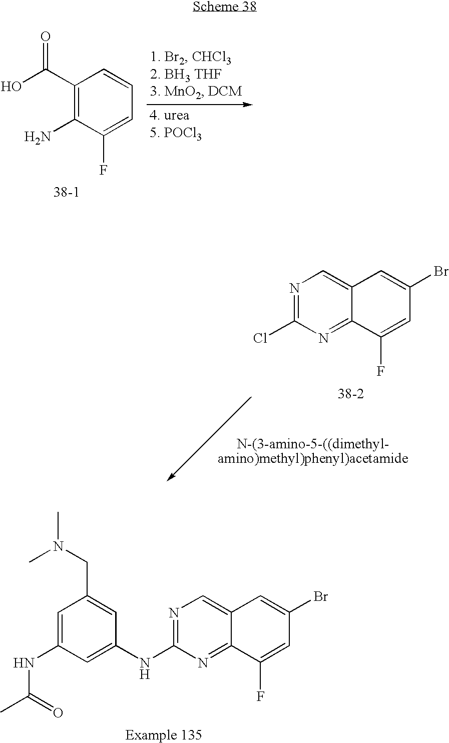

### Step 1: 2-Amino-5-bromo-3-fluorobenzoic acid

To a suspension of 2-amino-3-fluorobenzoic acid, 38-1, (5 g, 32.2 mmol) in chloroform (200 mL) was added dropwise bromine (1.1 equiv.) in chloroform (125 mL) solution. The mixture was stirred at RT for 16 hrs. The resulting white solid was collected by filtration and washed thoroughly with DCM until the filtrate was colorless. The solid was air-dried to give 9.6 g of white powder as the HBr salt of 2-amino-5-bromo-3-fluorobenzoic acid (95% yield). ES/MS m/z 234/236 (MH⁺).

### Step 2

To the above intermediate (30.6 mmol) in THF (100 mL) at 0° C. was added boron-THF complex solution (1 M in THF, 129 mL, 4 equiv.). The resulting mixture was stirred at RT for 40 hrs. The solvent was removed in vacuo and the excess reagent was quenched by the addition of water (30 mL) slowly. The pH (˜3) of the quenched mixture was adjusted to pH 7 by adding sodium bicarbonate (sat. aq.). The mixture was then extracted with DCM. The organic extracts were combined, washed with brine, dried with sodium sulfate and concentrated to provide a crude material as a white solid. ES/MS m/z 220/222 (MH⁺).

### Step 3: 2-Amino-5-bromo-3-fluorophenyl)methanol

To the above intermediate (30.6 mmol) in DCM (450 mL) was added manganese dioxide (MnO₂, 22 g, 258 mmol). The resulting mixture was stirred at RT under argon for 18 hrs. The mixture was then filtered through diatomaceous earth and washed thoroughly with DCM. The filtrate was then concentrated in vacuo to provide the crude product (2-amino-5-bromo-3-fluorophenyl)methanol (5.6 g) which was used for the next step without further purification. ES/MS m/z 218/220 (MH⁴).

### Step 4: 2-Hydroxyquinazoline

A mixture of (2-amino-5-bromo-3-fluorophenyl)methanol (5.6 g, 23.7 mmol, obtained from step 3) and urea (21 g, 15 equiv.) was heated to 175° C. with vigorous stirring for 15 min. The reaction was then cooled to RT and water was added. A solid precipitate formed and was collected by filtration and air-dried to provide 2-hydroxy-quinazoline as a light brown solid.

### Step 5: 6-Bromo-2-chloro-8-fluoroquinazoline, 38-2

To the above crude material was added phosphorus oxychloride (POCl₃, 20 mL) and this mixture was heated to 110° C. for 30 min. The mixture was then cooled to RT and concentrated in vacuo to nearly dryness. Ice water was added to the concentrate and the pH of the resulting mixture was adjusted to ˜6 using sodium bicarbonate. The pH adjusted mixture was extracted with DCM. The extract was dried with sodium sulfate and concentrated in vacuo to provide the desired product, 6-bromo-2-chloro-8-fluoroquinazoline, as light brown powder (1.63 g).

### Step 6: N-(3-(6-Bromo-8-fluoroquinazolin-2-ylamino)-5-((dimethylamino)methyl)-phenyl)acetamide

A mixture of 6-bromo-2-chloro-8-fluoroquinazoline, 38-2, N-(3-amino-5-((dimethylamino)methyl)phenyl)acetamide (1 equiv.) and HCl in dioxane (1 equiv.) in isopropanol (2.5 mL) was heated to 75° C. for 16 hrs. The resulting mixture was diluted with water, washed with ethyl acetate to remove organic impurities, basified the aqueous portion with sodium bicarbonate (aq.) to pH 9, and then brine was added. The basified aqueous solution was extracted with chloroform (3×). The organic extracts were combined, washed with brine, dried with sodium sulfate and concentrated to give a crude material which was purified by HPLC.

### Example 139

### (Table 4): N-(3-((Dimethylamino)methyl)-5-(6-ethynyl-8-fluoro-quinazolin-2-ylamino)phenyl)acetamide

The subject compound was prepared according to the general Scheme 39 below:

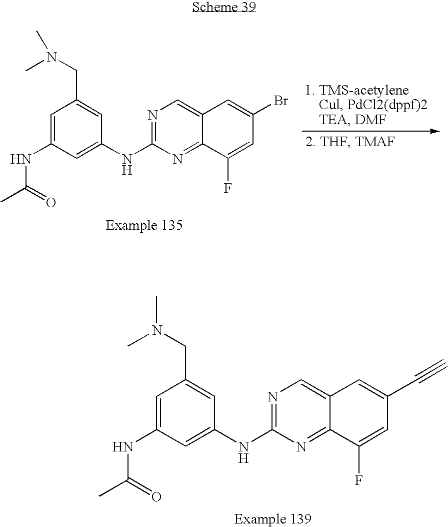

### Step 1

A mixture of N-(3-(6-bromo-8-fluoroquinazolin-2-ylamino)-5-((dimethylamino)-methyl)phenyl)acetamide, (from Example 135), ethynyltrimethylsilane, copper(I) iodide, 1,1′-bis(diphenylphosphino)ferrocenedichloro palladium(II), TEA and DMF, prepared according to the same stoichiometry as employed in the reaction of Example 7, step 1, is microwaved at 120° C. for 8 min. The resulting mixture is diluted with ethyl acetate, washed with water and brine, dried with sodium sulfate and concentrated to provide a crude residue.

### Step 2: N-(3-(8-Fluoro-6-ethynylquinazolin-2-ylamino)-5-((dimethylamino)methyl)-phenyl)acetamide (Example 139)

To the intermediate from Step 1 is added THF, 2-propanol and tetramethylammonium fluoride, according to the same stoichiometry as employed in the reaction of Example 7, step 2. The mixture is stirred at RT for 20 min. The resulting mixture is diluted with water and extracted with ethyl acetate (3×). The organic extracts are combined, washed with brine, dried with sodium sulfate and concentrated to provide a crude material which is purified by HPLC to yield the title compound.

### Example 216

### (Table 2): N-(3-(6-ethynyl-8-(piperidin-4-yloxy)quinazolin-2-ylamino)-5-(pyrimidin-5-yl)phenyl)acetamide

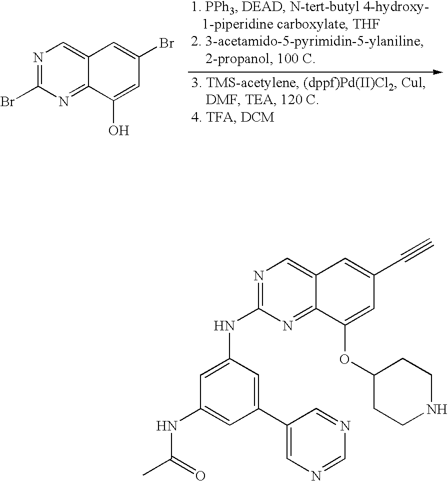

### Step 1: Preparation of 2,6-dibromo-8-(N-Boc-piperidin-4-yloxy)quinazoline

To a 0.30M solution of triphenylphosphine (2.0 eq) in THF was added diethylazodicarboxylate (2.0 eq). The mixture was stirred 15 min at ambient temperature. N-Tert-butyl-4-Hydroxy-1-piperidine carboxylate (4.0 eq) was added. The mixture was stirred 15 min at ambient temperature. 2,6-Dibromo-8-hydroxyquinazoline (1.0 eq) was added. The mixture was stirred an additional 24 h. The crude mixture was concentrated, purified by flash chromatography (2:1 hexanes:EtOAc), and concentrated to give the desired product.

### Step 2. Displacement

To a 0.30M solution of 2,6-dibromo-8-(N-Boc-piperidin-4-yloxy)quinazoline in 2-propanol was added 3-acetamido-5-pyrimidin-5-ylaniline (1.0 eq). The reaction was stirred at 100° C. for 14 h. The crude mixture was concentrated and used without further purification.

### Step 3. Sonogashira & Desilylation

The product from Step 2 was treated analogously to Example 281 step 2 and carried on to Step 4 without purification.

Step 4. Deprotection

The product from Step 3 was dissolved in enough 1:1 DCM:TFA to make a 0.20M solution. The mixture was stirred for 30 min at ambient temperature and concentrated. The crude product was purified by reverse-phase HPLC and lyophilized to give the desired product as its trifluoroacetic acid salt. ES/MS m/z 480 (MH⁺).

### Example 220

### (Table 2): 3-morpholino-5-(8-(piperidin-4-yloxy)-6-(1H-pyrazol-4-yl)quinazolin-2-ylamino)benzamide

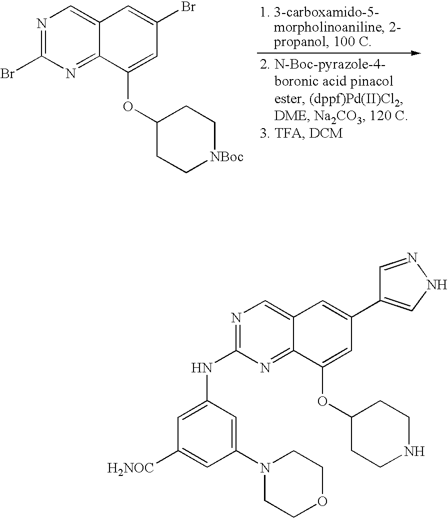

### Step 1. Displacement

To a 0.30M solution of the product from Example 283 step 1 in 2-propanol was added 3-carboxamido-5-morpholinoaniline (1.0 eq). The reaction was stirred at 100° C. for 14 h. The crude mixture was concentrated and used without further purification.

### Step 2. Suzuki

To a 0.10M solution of the product from Step 1 (1.0 eq) in DME was added N-Boc-pyrazole-4-boronic acid pinacol ester (4.0 eq), [1,1′-bis(diphenylphosphino)ferrocene]dichloropalladium(II) complex with DCM (0.10 eq), and 2.0M aqueous sodium carbonate (5.0 eq). The reaction was microwaved at 120 C for 10 min. The mixture was diluted with THF, filtered, concentrated, and carried on to Step 4 without purification.

### Step 3. Deprotection

The product from Step 2 was dissolved in enough 1:1 DCM:TFA to make a 0.20M solution. The mixture was stirred for 30 min at ambient temperature and concentrated. The crude product was purified by reverse-phase HPLC and lyophilized to give the desired product as its trifluoroacetic acid salt. ES/MS m/z 515 (MH³⁰).

### Example 229

### (Table 2): 3-morpholino-5-(8-(piperidin-4-yloxy)-6-(thiazol-2-yl)quinazolin-2-ylamino)benzamide

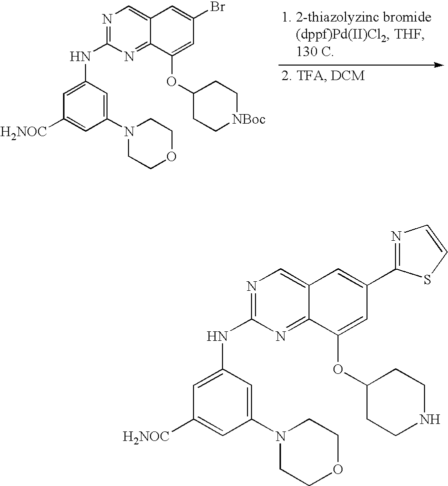

### Step 1. Negishi

To the product of Example 284 step 1 was added zinc(II) cyanide (4.0 eq) and [1,1′-bis(diphenylphosphino)ferrocene]dichloropalladium(II) complex with DCM (0.10 eq). The reaction was microwaved at 130° C. for 10 min. The mixture was diluted with ethyl acetate and washed with aqueous EDTA pH˜9 buffer. The organic phase was dried over sodium sulfate, and concentrated to give the desired product.

### Step 2. Deprotection

The product from Step 1 was dissolved in enough 1:1 DCM:TFA to make a 0.20M solution. The mixture was stirred for 30 min at ambient temperature and concentrated. The crude product was purified by reverse-phase HPLC and lyophilized to give the desired product as its trifluoroacetic acid salt. ES/MS m/z 532 (MH⁺).

### Example 340

### (Table 2): 2-(3-(6-ethynylquinazolin-2-ylamino)-5-(1-methyl-1H-pyrazol-4-yl)phenoxy)-N-(1-methylpiperidin-4-yl)acetamide

The subject compound was prepared according to the general Scheme below:

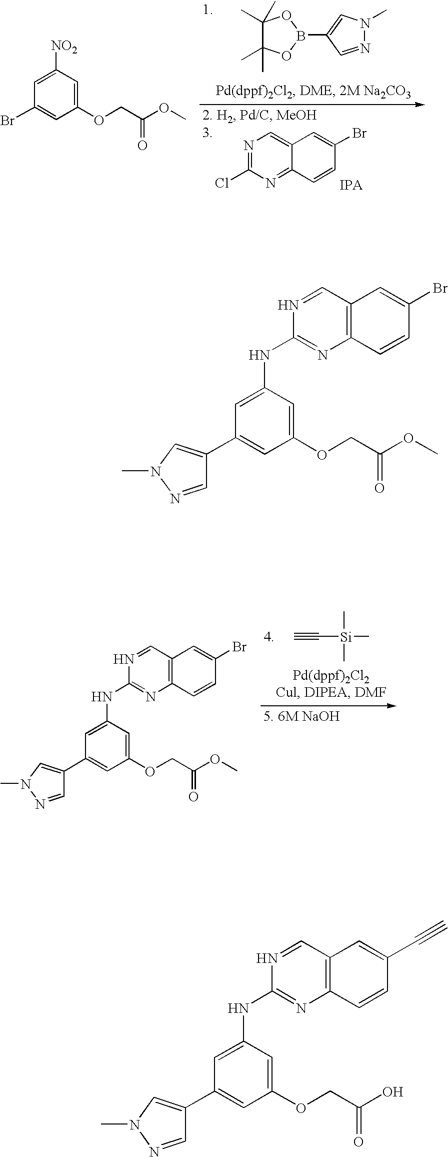

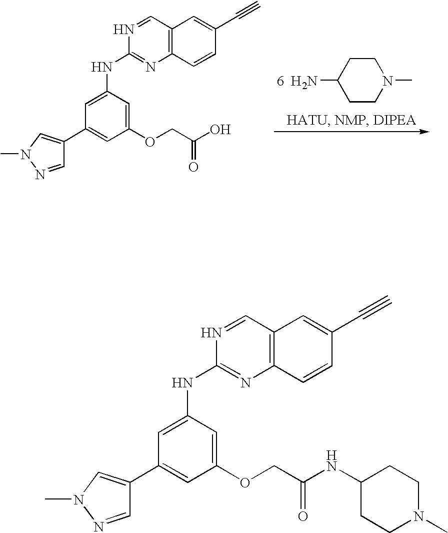

### Step 1: methyl 2-(3-(1-methyl-1H-pyrazol-4-yl)-5-nitrophenoxy)acetate

To the reaction mixture of methyl 2-(3-bromo-5-nitrophenoxy)acetate (1.65 g, 5.69 mmol) in 35 ml of DME was added Pd(dppf)₂Cl₂ (465 mg, 0.569 mmol), 1-methyl-4-(4,4,5,5-tetramethyl-1,3,2-dioxaborolan-2-yl)-1H-pyrazole (2.4 g, 11.4 mmol) and last add 2M Na₂CO₃ (11.4 ml, 22.8 mmol). This reaction mixture was stirred at 85° C. for 90 minutes or until done by LCMS. Concentrate about half of the DME off, add 350 ml of ethyl acetate and 50 ml of water. The organic layer was extracted and washed with saturated Na₂CO₃, water (2×), saturated NaCl, dried Na₂SO₄, filtered and concentrated to residue. The crude material was purified by silica gel column chromatography and concentrated in vaccuo to give, methyl 2-(3-(1-methyl-1H-pyrazol-4-yl)-5-nitrophenoxy)acetate, (480 mg). ES/MS m/z 292 (MH⁺).

### Step 2: methyl 2-(3-amino-5-(1-methyl-1H-pyrazol-4-yl)phenoxy)acetate

Analogous to Example 459, step 1 but using, methyl 2-(3-(1-methyl-1H-pyrazol-4-yl)-5-nitrophenoxy)acetate as starting material. ES/MS m/z 262 (MH⁺).

### Step 3: methyl 2-(3-(6-bromoquinazolin-2-ylamino)-5-(1-methyl-1H-pyrazol-4-yl)phenoxy)acetate

Analogous to Example 112, step 4 but using, methyl 2-(3-amino-5-(1-methyl-1H-pyrazol-4-yl)phenoxy)acetate as starting material. ES/MS m/z 468/470 (MH⁺).

### Step 4: methyl 2-(3-(1-methyl-1H-pyrazol-4-yl)-5-(6-((trimethylsilyl)ethynyl)quinazolin-2-ylamino)phenoxy)acetate

Analogous to Example 459, step 3 but using, methyl 2-(3-(6-bromoquinazolin-2-ylamino)-5-(1-methyl-1H-pyrazol-4-yl)phenoxy)acetate as starting material. ES/MS m/z 486 (MH⁺).

### Step 5: 2-(3-(6-ethynylquinazolin-2-ylamino)-5-(1-methyl-1H-pyrazol-4-yl)phenoxy)acetic acid

To the reaction mixture of, methyl 2-(3-(1-methyl-1H-pyrazol-4-yl)-5-(6-((trimethylsilyl)ethynyl)quinazolin-2-ylamino)phenoxy)acetate (740 mg, 1.52 mmol) in 6 ml of THF and 3 ml of MeOH add 6M NaOH (0.75 ml, 4.52 mmol) stir at room temperature for 1 hour or until done by LCMS add more 6 M NaOH if necessary. The reaction mixture was concentrated in vaccuo until dry to give crude residue. To this residue add 6M HCl aq (0.91 ml, 5.48 mmol) stir briefly and concentrated in vaccuo until dry to give, 2-(3-(6-ethynylquinazolin-2-ylamino)-5-(1-methyl-1H-pyrazol-4-yl) phenoxy)acetic acid, as crude solid used in next step (840 mg). ES/MS m/z 400 (MH⁺).

### Step 6: 2-(3-(6-ethynylquinazolin-2-ylamino)-5-(1-methyl-1H-pyrazol-4-yl)phenoxy)-N-(1-methylpiperidin-4-yl)acetamide

To the reaction mixture of, 2-(3-(6-ethynylquinazolin-2-ylamino)-5-(1-methyl-1H-pyrazol-4-yl)phenoxy)acetic acid (24 mg, 0.060 mmol) in 0.5 ml of NMP add HATU (39 mg, 0.102 mmol), DIPEA (0.023 ml, 0.132 mmol) and stir at room temperature for about 3 minutes. To the above reaction mixture add 1-methylpiperidin-4-amine (27 mg, 0.24 mmol) and stir at room temperature for 4 hours or until done by LCMS. The crude reaction mixture was filtered, purified on prep HPLC and lyophilized to give, 2-(3-(6-ethynylquinazolin-2-ylamino)-5-(1-methyl-1H-pyrazol-4-yl)phenoxy)-N-(1-methylpiperidin-4-yl)acetamide as TFA salt (7.6 mg). ES/MS m/z 496 (MH⁺).

### Example 356

### (Table 2): 2-(3-(6-cyanoquinazolin-2-ylamino)-5-(1-methyl-1H-pyrazol-4-yl)phenoxy)-N-methylacetamide

The subject compound was prepared according to the general Scheme below:

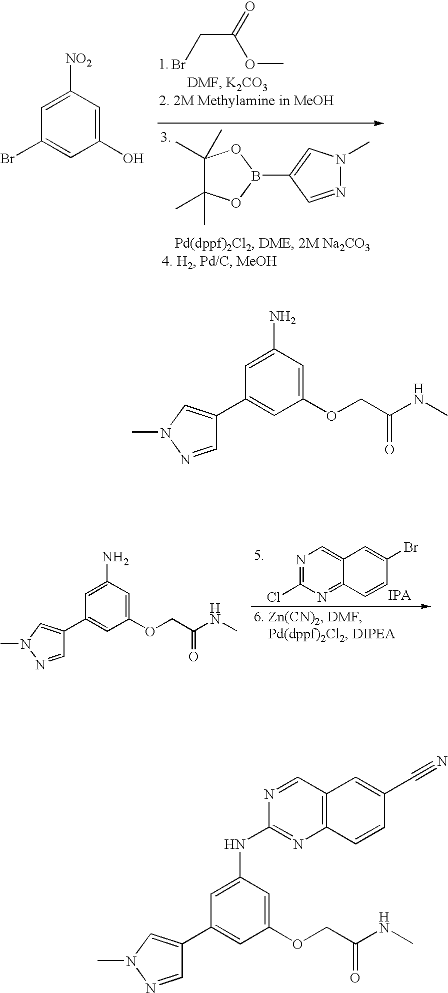

### Step 1: methyl 2-(3-bromo-5-nitrophenoxy)acetate

To the reaction mixture of 3-bromo-5-nitrophenol (7.0 g, 32.1 mmol) in 30 ml of DMF add K₂CO₃ (9.8 g, 70.6 mmol) and stir for 3-5 minutes. To the reaction mixture add methyl 2-bromoacetate (5.4 g, 35.3 mmol) and stir at room temperature 18 hours or until done by LC. To the crude reaction add 450 ml of ethyl acetate and wash with saturated Na₂CO₃, water (3×), saturated NaCl, dried Na₂SO₄, filtered and concentrated in vaccuo to give crude, methyl 2-(3-bromo-5-nitrophenoxy)acetate, (9.0 g).

### Step 2: 2-(3-bromo-5-nitrophenoxy)-N-methylacetamide

To the crude product from step 1, methyl 2-(3-bromo-5-nitrophenoxy)acetate (1.75 g, 6.0 mmol) add 2M methylamine in methanol (18 ml, 36 mmol) and stir at room temperature 24 hours or until done by LCMS. The crude reaction mixture was concentrated in vaccuo to give crude, 2-(3-bromo-5-nitrophenoxy)-N-methylacetamide, (1.74 g). ES/MS m/z 289/291 (MH⁺).

### Step 3: N-methyl-2-(3-(1-methyl-1H-pyrazol-4-yl)-5-nitrophenoxy)acetamide

To the reaction mixture of 2-(3-bromo-5-nitrophenoxy)-N-methylacetamide (800 mg, 2.77 mmol) in 17 ml of DME was added Pd(dppf)₂Cl₂(226 mg, 0.277 mmol), 1-methyl-4-(4,4,5,5-tetramethyl-1,3,2-dioxaborolan-2-yl)-1H-pyrazole (1.04 g, 5.0 mmol) and last add 2M Na₂CO₃ (5.5 ml, 11 mmol). This reaction mixture was stirred at 85-90° C. for 18 hours or until done by LCMS. Concentrate most of the DME off, add about 200 ml of ethyl acetate and 50 ml of water and stir. The solids (product) were collected by filtration, washed with water (1×) and dried under vacuum to give crude, N-methyl-2-(3-(1-methyl-1H-pyrazol-4-yl)-5-nitrophenoxy)acetamide, (660 mg). ES/MS m/z 291 (MH⁺).

### Step 4: 2-(3-amino-5-(1-methyl-1H-pyrazol-4-yl)phenoxy)-N-methylacetamide

To the starting crude material, N-methyl-2-(3-(1-methyl-1H-pyrazol-4-yl)-5-nitrophenoxy)acetamide (660 mg, 2.27 mmol) was added 10% Pd on Carbon (132 mg, 20% by wt.) under argon. Under argon with a syringe carefully add 5 ml methanol. To this reaction mixture was added a hydrogen balloon and was evacuated and refilled 5 times. The reaction was stirred at room temperature for 22 hours or until done by LC.

To the reaction mixture add ethyl acetate and under argon filtered through celite and washed with a 1:1 solution of ethyl acetate and methanol. The filtrate was concentrated in vaccuo to give, 2-(3-amino-5-(1-methyl-1H-pyrazol-4-yl)phenoxy)-N-methylacetamide as crude material used in next step. (565 mg). ES/MS m/z 261 (MH^{÷}).

### Step 5: 2-(3-(6-bromoquinazolin-2-ylamino)-5-(1-methyl-1H-pyrazol-4-yl)phenoxy)-N-methylacetamide

Analogous to Example 112, step 4 but using, 2-(3-amino-5-(1-methyl-1H-pyrazol-4-yl)phenoxy)-N-methylacetamide as starting material. ES/MS m/z 467/469 (MH⁺).

### Step 6: 2-(3-(6-cyanoquinazolin-2-ylamino)-5-(1-methyl-1H-pyrazol-4-yl)phenoxy)-N-methylacetamide

To the reaction mixture of 2-(3-(6-bromoquinazolin-2-ylamino)-5-(1-methyl-1H-pyrazol-4-yl)phenoxy)-N-methylacetamide (30 mg, 0.064 mmol) in 0.6 ml of DMF was added Pd(dppf)₂Cl₂ (10.5 mg, 0.0128 mmol), Zn(CN)₂ (30 mg, 0.256 mmol) and DIPEA (34 ul, 0.192 mmol). This reaction mixture was microwaved at 170° C. for 800 seconds then again at 210° C. for 800 seconds. The crude reaction mixture was filtered, purified on prep HPLC and lyophilized to give, 2-(3-(6-cyanoquinazolin-2-ylamino)-5-(1-methyl-1H-pyrazol-4-yl)phenoxy)-N-methylacetamide as TFA salt (8.2 mg). ES/MS m/z 414 (MH⁺).

### Example 379

### (Table 2): tert-butyl 3-(6-bromoquinazolin-2-ylamino)benzylcarbamate

The subject compound was prepared according to the general Scheme below:

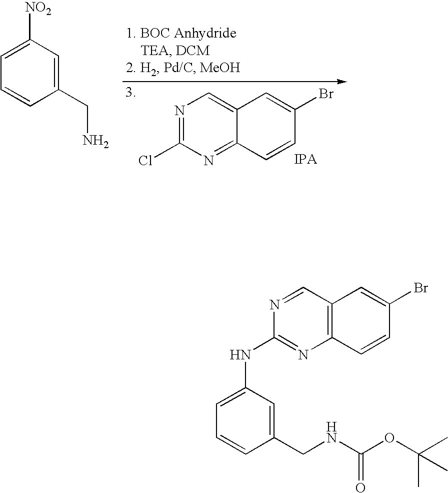

### Step 1: tert-butyl 3-nitrobenzylcarbamate

To the reaction mixture of (3-nitrophenyl)methanamine HCl (2.00 g, 10.6 mmol) in 15 ml of DCM was added TEA (3.7 ml, 26.5 mmol) and di-tert-butyl dicarbonate (2.78 g, 12.72 mmol). The reaction was stirred at room temperature for 90 minutes or until done by LC. Concentrate most of the DCM of add 200 ml ethyl acetate and washed with sat. NaHCO₃ (2×), water (2×), brine, dried with Na₂SO₄, filtered and concentrated in vaccuo to give, tert-butyl 3-nitrobenzylcarbamate as crude material used in next step. (2.96 grams). ES/MS m/z 253 (MH⁺).

### Step 2: tert-butyl-aminobenzylcarbamate

To the starting crude material tert-butyl 3-nitrobenzylcarbamate (2.96 g crude, 10.6 mmol) was added 10% Pd on Carbon (444 mg, 15% by wt.) under argon. Under argon with a syringe carefully add 19 ml methanol. To this reaction mixture was added a hydrogen balloon and was evacuated and refilled 5 times. The reaction was stirred at room temperature for 18 hours or until done by LC. To the reaction mixture add ethyl acetate and under argon filtered through celite and washed with a 1:1 solution of ethyl acetate and methanol. The filtrate was concentrated to residue. To the residue add 150 ml ethyl acetate and wash with sat. NaHCO₃ (2×), water (2×), brine, dried with Na₂SO₄, filtered and concentrated in vaccuo to give, tert-butyl 3-aminobenzylcarbamate as crude material used in next step. (2.45 grams). ES/MS m/z 223 (MH⁺).

### Step 3: tert-butyl 3-(6-bromoquinazolin-2-ylamino)benzylcarbamate

To the reaction mixture of tert-butyl 3-aminobenzylcarbamate (840 mg, 3.76 mmol) in 6 ml of IPA in a glass bomb was added 6-bromo-2-chloroquinazoline (750 mg, 3.08 mmol) and caped. The reaction solution was stirred at 95° C. for 20 hours or until done by LCMS. To the crude reaction mixture add 6 ml of IPA, filter solids off and the concentrate the filtrate (product). The crude material was purified by silica gel column chromatography and concentrated in vaccuo to give, tert-butyl 3-(6-bromoquinazolin-2-ylamino)benzylcarbamate (860 mg). ES/MS m/z 429/431 (MH⁺) Additional purification can be done by prep HPLC, lyophilized and converted to an TFA ammonium salt.

### Example 400

### (Table 2): N-(2-(3-(6-ethynylquinazolin-2-ylamino)-5-(1-methyl-1H-pyrazol-4-yl)phenoxy)ethyl)acetamide

The subject compound was prepared according to the general Scheme below:

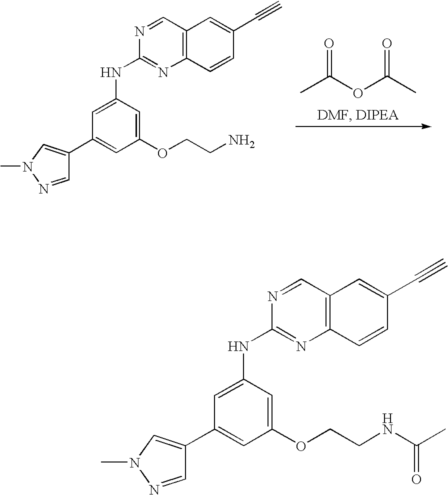

To the reaction mixture of N-(3-(2-aminoethoxy)-5-(1-methyl-1H-pyrazol-4-yl)phenyl)-6-ethynylquinazolin-2-amine (21 mg, 0.055 mmol) in 0.65 ml of DMF add DIPEA (0.035 ml, 0.195 mmol), and acetic anhydride (11.3 mg, 0.111 mmol). The reaction mixture was stirred at room temperature for about 90 minutes or until done by LCMS. The crude reaction mixture was filtered, purified on prep HPLC and lyophilized to give, N-(2-(3-(6-ethynylquinazolin-2-ylamino)-5-(1-methyl-1H-pyrazol-4-yl)phenoxy)ethyl)acetamide as TFA salt (4.3 mg). ES/MS m/z 427 (MH⁺).

### Example 459

### (Table 2): 6-ethynyl-N-(3-(1-methyl-1H-pyrazol-4-yl)-5-(2-(pyrrolidin-1-yl)ethoxy)phenyl)quinazolin-2-amine

The subject compound was prepared according to the general Scheme, below:

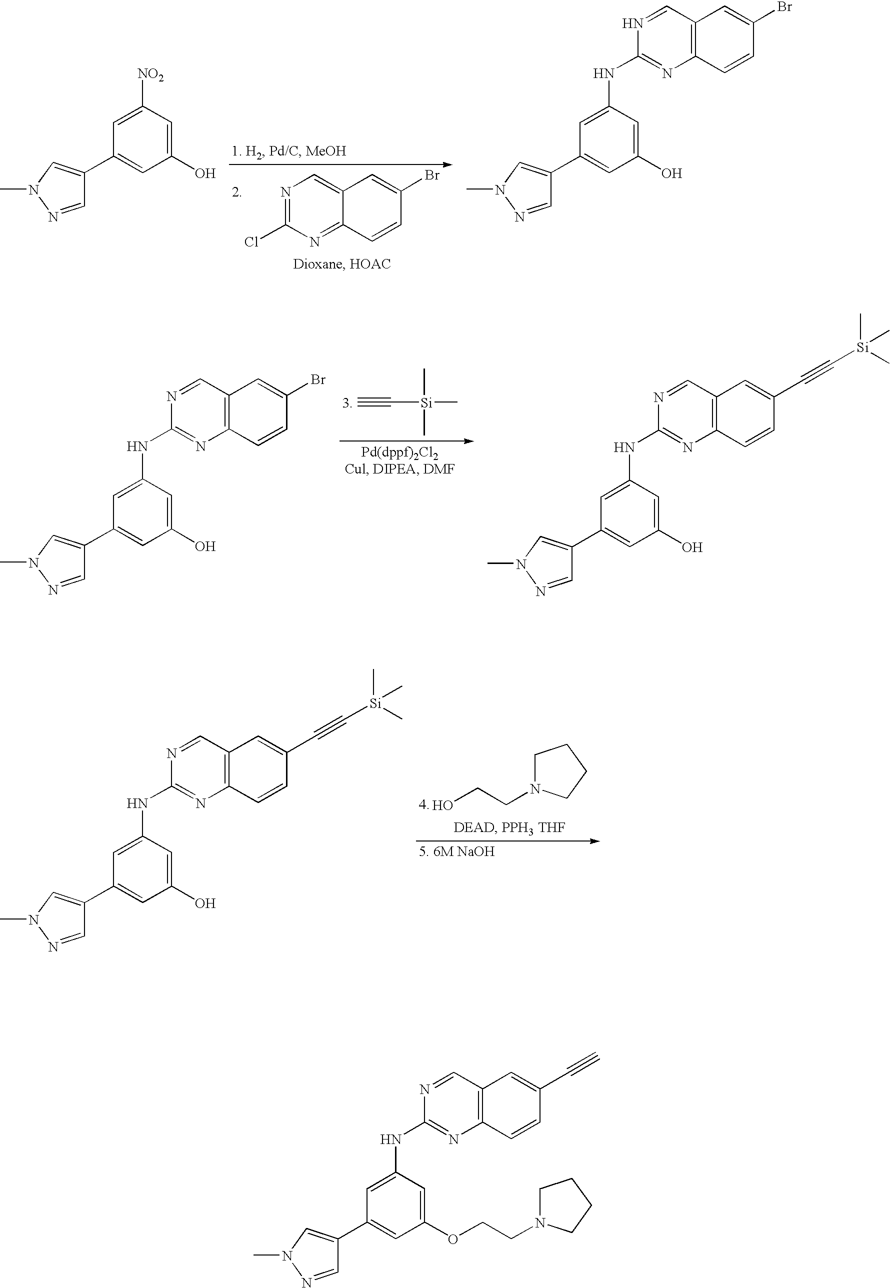

### Step 1: 3-amino-5-(1-methyl-1H-pyrazol-4-yl)phenol

To the starting crude material 3-(1-methyl-1H-pyrazol-4-yl)-5-nitrophenol (7.04 g, 32 mmol) was added 10% Pd on Carbon (2.1 g, 30% by wt.) under argon. Under argon with a syringe carefully add 95 ml methanol. To this reaction mixture was added a hydrogen balloon and was evacuated and refilled 6 times. The reaction was stirred at room temperature for 22 hours or until done by LC. To the reaction mixture add ethyl acetate and under argon filtered through celite and washed with a 1:1 solution of ethyl acetate and methanol. The filtrate was concentrated under reduced pressure to give 3-amino-5-(1-methyl-1H-pyrazol-4-yl)phenol, as crude material (8.0 grams). ES/MS m/z 190 (MH⁺).

### Step 2: 3-(6-bromonaphthalen-2-ylamino)-5-(1-methyl-1H-pyrazol-4-yl)phenol

To the reaction mixture of 3-amino-5-(1-methyl-1H-pyrazol-4-yl)phenol (2.6 g, 13.68 mmol) in 20 ml of dioxane was added 6-bromo-2-chloroquinazoline (1.75 g, 7.20 mmol) and acetic acid (1.73 ml, 28.8 mmol). The reaction solution was stirred at 95-100° C. for 60 hours or until done by LCMS. To the crude reaction mixture add 30 ml dioxane, let cool, filter solids off and the concentrate the filtrate (product). The crude residue was purified by silica gel column chromatography and concentrated in vaccuo to give 3-(6-bromonaphthalen-2-ylamino)-5-(1-methyl-1H-pyrazol-4-yl)phenol, (1.13 grams). ES/MS m/z 396/398 (MH⁺). Additional purification can be done by prep HPLC and lyophilized to make a TFA salt.

### Step 3: 3-(1-methyl-1H-pyrazol-4-yl)-5-(6-((trimethylsilyl)ethynyl)quinazolin-2-ylamino)phenol

To the reaction mixture of 3-(6-bromonaphthalen-2-ylamino)-5-(1-methyl-1H-pyrazol-4-yl)phenol (1.13 g, 2.85 mmol) in 7 ml of DMF was added Pd(dppf)₂Cl₂(232 mg, 0.28 mmol), CuI (119 mg, 0.622 mmol), ethynyltrimethylsilane (838 mg, 8.55 mmol) and last add N-ethyl-N-isopropylpropan-2-amine (DIPEA) (1.5 ml, 8.55 mmol). This reaction mixture was stirred at 95° C. for 1 hour or until done by LCMS. Concentrate most of the DMF off, add 350 ml of ethyl acetate, 75 ml of saturated sodium bicarbonate and briefly stirred. The mixture formed an emulsion that was filtered through celite and flushed with 100 ml ethyl acetate. The organic layer was extracted and washed with water, saturated NaCl, dried with Na₂SO₄, filtered through a 2″×3″ silica gel plug, flushed with ethyl acetate and concentrated in vaccuo to give 3-(1-methyl-1H-pyrazol-4-yl)-5-(6-((trimethylsilyl)ethynyl)quinazolin-2-ylamino)phenol, (1.20 grams). ES/MS m/z 414 (MH⁺).

### Step 4: N-(3-(1-methyl-1H-pyrazol-4-yl)-5-(2-pyrrolidin-1-yl)ethoxy)phenyl)-6-((trimethylsilyl)ethynyl)quinazolin-2-amine

To the reaction mixture of triphenyl phosphine (32 mg, 0.12 mmol) in 0.5 ml of THF add 2-(pyrrolidin-1-yl)ethanol (17.3 mg, 0.15 mmol), then add DEAD (21 mg, 0.12 mmol) and stir at room temperature for 10 minutes. The above reaction mixture was added to 3-(1-methyl-1H-pyrazol-4-yl)-5-(6-((trimethylsilyl)ethynyl)quinazolin-2-ylamino)phenol (24.8 mg, 0.06 mmol) and stirred at room temperature for 20 hours or until done by LCMS. The reaction mixture was concentrated under reduced pressure to give N-(3-(1-methyl-1H-pyrazol-4-yl)-5-(2-(pyrrolidin-1-yl)ethoxy)phenyl)-6-((trimethylsilyl)ethynyl)quinazolin-2-amine as a crude residue used in the next step. ES/MS m/z 511 (MH⁺).

### Step 5: 6-ethynyl-N-(3-(1-methyl-1H-pyrazol-4-yl)-5-(2-(pyrrolidin-1-yl)ethoxy)phenyl)quinazolin-2-amine

To the crude reaction mixture of N-(3-(1-methyl-1H-pyrazol-4-yl)-5-(2-(pyrrolidin-1-yl)ethoxy)phenyl)-6-((trimethylsilyl)ethynyl)quinazolin-2-amine (0.06 mmol) in 1 ml of DMF was added 6M NaOH (0.06 ml, 0.36 mmol) and stirred at room temperature for 10 minutes and checked by LCMS. If the de-protection is incomplete add more 6 M NaOH and recheck in 10 minutes. The crude reaction mixture was filtered, purified on prep HPLC and lyophilized to give 6-ethynyl-N-(3-(1-methyl-1H-pyrazol-4-yl)-5-(2-(pyrrolidin-1-yl)ethoxy)phenyl)quinazolin-2-amine as TFA salt (4.4 mg). ES/MS m/z 439 (MH⁺).

### Example 469

### (Table 2): N-(2-(3-(6-ethynylquinazolin-2-ylamino)-5-(1-methyl-1H-pyrazol-4-yl)phenoxy)ethyl)-1-methyl-1H-pyrazole-4-carboxamide

The subject compound was prepared according to the general Scheme, below:

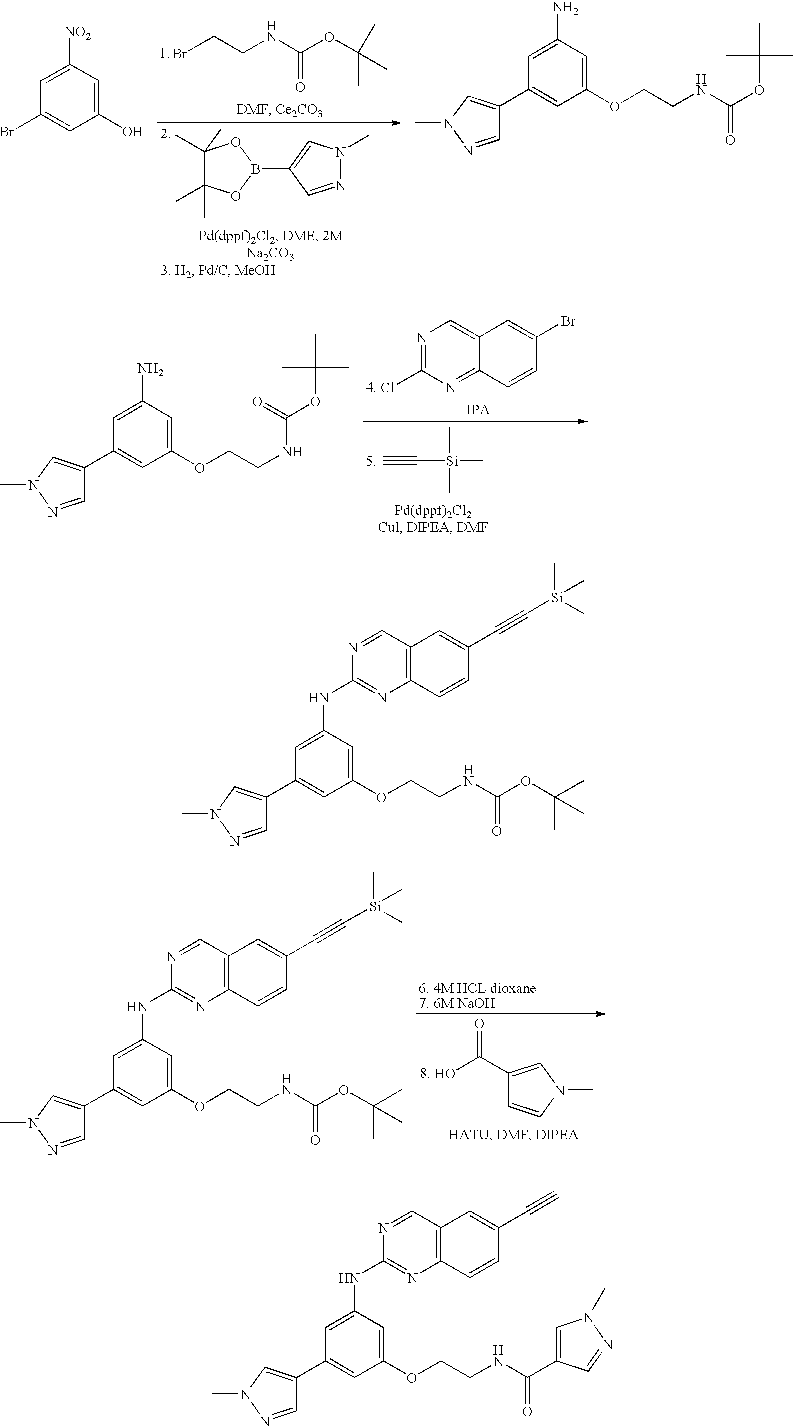

### Step 1: tert-butyl 2-(3-bromo-5-nitrophenoxy)ethylcarbamate

To the reaction mixture of 3-bromo-5-nitrophenol (4.0 g, 18.35 mmol) in 40 ml of DMF add cesium carbonate (12.0 g, 367 mmol) and stir at room temperature for 30 minutes. To the above reaction mixture was added tert-butyl 2-bromoethylcarbamate (6.16 g, 27.5 mmol), cap with argon balloon and stirred at 40° C. for 18 hours or until done by LCMS. Concentrate about half of the DMF off, add 500 ml of ethyl acetate, wash organic layer 1M NaOH (3×), water, saturated NaCl, dried with Na₂SO₄, filtered and concentrated in vaccuo to give crude, tert-butyl 2-(3-bromo-5-nitrophenoxy)ethylcarbamate (6.6 grams) used in the next reaction.

### Step 2: tert-butyl 2-(3-(1-methyl-1H-pyrazol-4-yl)-5-nitrophenoxy)ethylcarbamate

To the reaction mixture of above crude tert-butyl 2-(3-bromo-5-nitrophenoxy)ethylcarbamate (6.6 g, 18.28 mmol) in 125 ml of DME was added Pd(dppf)₂Cl₂ (1.2 g, 1.46 mmol), 1-methyl-4-(4,4,5,5-tetramethyl-1,3,2-dioxaborolan-2-yl)-1H-pyrazole (5.7 g, 27.4 mmol) and last add 2M Na₂CO₃ (41.3 ml, 82.5 mmol). This reaction mixture was stirred at 100° C. for 2 hours or until done by LCMS. Concentrate most of the DME off, add 600 ml of ethyl acetate, 100 ml of water and briefly stirred. The mixture formed an emulsion that was filtered. The organic layer was extracted and washed with 1M NaOH (2×), water, saturated NaCl, dried Na₂SO₄, filtered and concentrated in vaccuo to give tert-butyl 2-(3-(1-methyl-1H-pyrazol-4-yl)-5-nitrophenoxy)ethylcarbamate, (6.2 grams). ES/MS m/z 363 (MH⁺).

### Step 3: tert-butyl 2-(3-amino-5-(1-methyl-1H-pyrazol-4-yl)phenoxy)ethylcarbamate

Analogous to Example 459, step 1 but using tert-butyl 2-(3-(1-methyl-1H-pyrazol-4-yl)-5-nitrophenoxy)ethylcarbamate as starting material. ES/MS m/z 333 (MH⁺).

### Step 4: tert-butyl 2-(3-(6-bromoquinazolin-2-ylamino)-5-(1-methyl-1H-pyrazol-4-yl)phenoxy)ethylcarbamate

To the reaction mixture of tert-butyl 2-(3-amino-5-(1-methyl-1H-pyrazol-4-yl)phenoxy)ethylcarbamate (5.53 g, 16.66 mmol) in 50 ml of IPA in a glass bomb was added 6-bromo-2-chloroquinazoline (3.9 g, 16.66 mmol) and caped. The reaction solution was stirred at 95-100° C. for 22 hours or until done by LCMS. To the crude reaction mixture add 20 ml of IPA, let cool, collect solids (product) wash 2×IPA. The crude solid was dried in vaccuo to give, tert-butyl 2-(3-(6-bromoquinazolin-2-ylamino)-5-(1-methyl-1H-pyrazol-4-yl)phenoxy)ethylcarbamate, (6.88 grams). ES/MS m/z 539/541 (MH⁺). Additional purification can be done by prep HPLC and lyophilized to make TFA salt.

### Step 5: tert-butyl 2-(3-(1-methyl-1H-pyrazol-4-yl)-5-(6-((trimethylsilyl)ethynyl)quinazolin-2-ylamino)phenoxy)ethylcarbamate

To the reaction mixture of tert-butyl 2-(3-(6-bromoquinazolin-2-ylamino)-5-(1-methyl-1H-pyrazol-4-yl)phenoxy)ethylcarbamate (6.0 g, 11.13 mmol) in 38 ml of DMF was added Pd(dppf)₂Cl₂(1.09 g, 1.36 mmol), CuI (529 mg, 2.78 mmol), ethynyltrimethylsilane (3.3 g, 33.4 mmol) and last add DIPEA (5.81 ml, 33.4 mmol). This reaction mixture was stirred at 95° C. for 1 hour or until done by LCMS. The BOC group may come partially off, if so add di-tert-butyl dicarbonate (1.5 g, 6.88 mmol) and stir at room temperature 30 minutes, re-check LCMS, add more if needed. Concentrate most of the DMF off, add 750 ml of ethyl acetate, 200 ml of saturated sodium bicarbonate and shake. The mixture formed an emulsion that was filtered, as necessary. The organic layer was extracted and washed with water (2×), saturated NaCl, dried Na₂SO₄, filtered through a 3.5″×3″ silica gel plug, flushed with ethyl acetate and concentrated in vaccuo to give, tert-butyl 2-(3-(1-methyl-1H-pyrazol-4-yl)-5-(6-((trimethylsilyl)ethynyl)quinazolin-2-ylamino)phenoxy)ethylcarbamate (5.45 grams). ES/MS m/z 557 (MH⁺).

### Step 6: N-(3-(2-aminoethoxy)-5-(1-methyl-1H-pyrazol-4-yl)phenyl)-6-((trimethylsilyl)ethynyl)quinazolin-2-amine

To the reaction mixture of tert-butyl 2-(3-(1-methyl-1H-pyrazol-4-yl)-5-(6-((trimethylsilyl)ethynyl)quinazolin-2-ylamino)phenoxy)ethylcarbamate (1.3 g, 2.3 mmol) add excess 4 M HCl in Dioxane (20 ml, 80 mmol). This reaction mixture was stirred at room temperature for 1 hour or until done by LCMS and concentrated in vaccuo to give crude, N-(3-(2-aminoethoxy)-5-(1-methyl-1H-pyrazol-4-yl)phenyl)-6-((trimethylsilyl)ethynyl)quinazolin-2-amine, (1.49 grams). ES/MS m/z 457 (MH⁺).

### Step 7: N-(3-(2-aminoethoxy)-5-(1-methyl-1H-pyrazol-4-yl)phenyl)-6-ethynylquinazolin-2:amine

To the above crude reaction mixture of, N-(3-(2-aminoethoxy)-5-(1-methyl-1H-pyrazol-4-yl)phenyl)-6-((trimethylsilyl)ethynyl)quinazolin-2-amine (2.33 mmol) in 20 ml of THF and 12 ml of MeOH add 6M NaOH (1.16 ml, 6.99 mmol) stir at room temperature for 15 minutes or until done by LCMS add more 6 M NaOH if necessary. The reaction mixture was concentrated in vaccuo until dry to give crude, N-(3-(2-aminoethoxy)-5-(1-methyl-1H-pyrazol-4-yl)phenyl)-6-ethynylquinazolin-2-amine, (1.72 grams). ES/MS m/z 385 (MH⁺).

### Step 8: N-(2-(3-(6-ethynylquinazolin-2-ylamino)-5-(1-methyl-1H-pyrazol-4-yl)phenoxy)ethyl)-1-methyl-1H-pyrazole-4-carboxamide

To the reaction mixture of 1-methyl-1H-pyrazole-4-carboxylic acid (11 mg, 0.0875 mmol) in 0.65 ml of NMP add HATU (40 mg, 0.105 mmol), DIPEA (0.031 ml, 0.175 mmol) and stir at room temperature for about 3 minutes. To the above reaction mixture add N-(3-(2-aminoethoxy)-5-(1-methyl-1H-pyrazol-4-yl)phenyl)-6-ethynylquinazolin-2-amine (22.4 mg, 0.058 mmol) and stir at room temperature for 2 hours or until done by LCMS. The crude reaction mixture was filtered, purified on prep HPLC and lyophilized to give N-(2-(3-(6-ethynylquinazolin-2-ylamino)-5-(1-methyl-1H-pyrazol-4-yl)phenoxy)ethyl)-1-methyl-1H-pyrazole-4-carboxamide as TFA salt (2.9 mg). ES/MS m/z 493 (MH⁺).

### Example 491

### (Table 2): (R)-2-amino-N-(2-(3-(6-ethynylquinazolin-2-ylamino)-5-(1-methyl-1H-pyrazol-4-yl)phenoxy)ethyl)propanamide

The subject compound was prepared according to the general Scheme below:

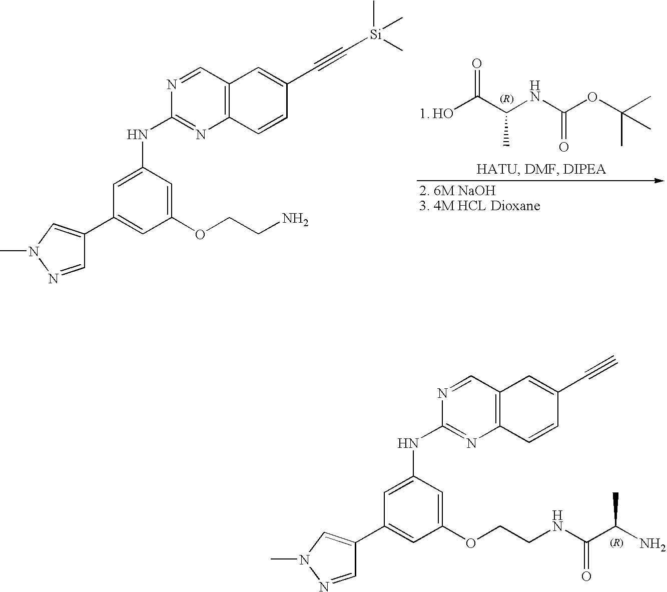

### Step 1: (R)-tert-butyl 1-(2-(3-(1-methyl-1H-pyrazol-4-yl)-5-(6-((trimethylsilyl)ethynyl)quinazolin-2-ylamino)phenoxy)ethylamino)-1-oxopropan-2-ylcarbamate

To the reaction mixture of (R)-2-(tert-butoxycarbonylamino)propanoic acid (25.5 mg, 0.135 mmol) in 0.15 ml of DMF add HATU (55 mg, 0.144 mmol), DIPEA (0.028 ml, 0.16 mmol) and stir at room temperature for about 10 minutes. To the above reaction mixture add a solution of N-(3-(2-aminoethoxy)-5-(1-methyl-1H-pyrazol-4-yl)phenyl)-6-((trimethylsilyl)ethynyl)quinazolin-2-amine (41 mg, 0.09 mmol) in 0.5 ml DMF with DIPEA (0.028 ml, 0.16 mmol) and stir at room temperature for 18 hours or until done by LCMS. The crude reaction solution with the product (R)-tert-butyl 1-(2-(3-(1-methyl-1H-pyrazol-4-yl)-5-(6-((trimethylsilyl)ethynyl)quinazolin-2-ylamino)phenoxy)ethylamino)-1-oxopropan-2-ylcarbamate, was used in the next step without purification. ES/MS m/z 628 (MH⁺).

### Step 2: (R)-tert-butyl 1-(2-(3-(6-ethynylquinazolin-2-ylamino)-5-(1-methyl-1H-pyrazol-4-yl)phenoxy)ethylamino)-1-oxopropan-2-ylcarbamate

To the above crude material (R)-tert-butyl 1-(2-(3-(1-methyl-1H-pyrazol-4-yl)-5-(6-((trimethylsilyl)ethynyl)quinazolin-2-ylamino)phenoxy)ethylamino)-1-oxopropan-2-ylcarbamate (0.09 mmol) add 6 M NaOH (0.120 ml, 0.72 mmol) and stir at room temperature for 10 minutes or until done by LCMS. If the reaction is not complete add more 6M NaOH as needed. The crude reaction mixture with product, (R)-tert-butyl 1-(2-(3-(6-ethynylquinazolin-2-ylamino)-5-(1-methyl-1H-pyrazol-4-yl)phenoxy)ethylamino)-1-oxopropan-2-ylcarbamate was use in the next step without purification: ES/MS m/z 556 (MH⁺).

### Step 3: (R)-2-amino-N-(2-(3-(6-ethynylquinazolin-2-ylamino)-5-(1-methyl-1H-pyrazol-4-yl)phenoxy)ethyl)propanamide

To the above crude material (R)-tert-butyl 1-(2-(3-(6-ethynylquinazolin-2-ylamino)-5-(1-methyl-1H-pyrazol-4-yl)phenoxy)ethylamino)-1-oxopropan-2-ylcarbamate (0.09 mmol) add 4 M HCl in dioxane (3 ml, 12 mmol) and stir at room temperature for 1 hour. The reaction mixture was concentrated, 1 ml DMF added, filtered, purified on prep HPLC and lyophilized to give (R)-2-amino-N-(2-(3-(6-ethynylquinazolin-2-ylamino)-5-(1-methyl-1H-pyrazol-4-yl)phenoxy)ethyl)propanamide, as TFA salt (14.1 mg). ES/MS m/z 456 (MH⁺).

### Example 495

### (Table 2): (R)-2-(dimethylamino)-N-(2-(3-(6-ethynylquinazolin-2-ylamino)-5-(1-methyl-1H-pyrazol-4-yl)phenoxy)ethyl)propanamide

The subject compound was prepared according to the general Scheme, below:

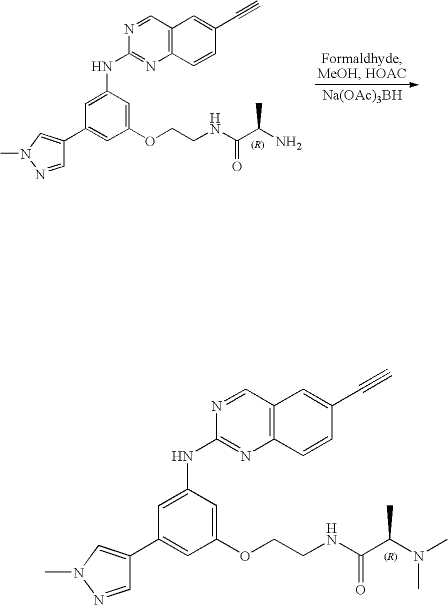

To the reaction mixture of (R)-2-amino-N-(2-(3-(6-ethynylquinazolin-2-ylamino)-5-(1-methyl-1H-pyrazol-4-yl)phenoxy)ethyl)propanamide (12 mg, 0.026 mmol) in 1.2 ml of methanol add acetic acid (0.08 ml, 1.3 mmol) and 37% formaldehyde solution in water (0.034 ml, 0.39 mmol). The reaction mixture was stirred at room temperature for about 20 minutes. To this reaction solution was added sodium triacetoxy borohydride (44 mg, 0.208 mmol) and stirred at room temperature 1-2 hours or until done by LCMS. The crude reaction mixture was concentrated, 1 ml of DMF added, filtered, purified on prep HPLC and lyophilized to give, (R)-2-(dimethylamino)-N-(2-(3-(6-ethynylquinazolin-2-ylamino)-5-(1-methyl-1H-pyrazol-4-yl)phenoxy)ethyl)propanamide as TFA salt (1.1 mg). ES/MS m/z 484 (MH⁺).

### Example 506

### (Table 2): N-(2-(3-(6-ethynylquinazolin-2-ylamino)-5-(1-methyl-1H-pyrazol-4-yl)phenoxy)ethyl)-2-(piperidin-1-yl)acetamide

The subject compound was prepared according to the general Scheme below:

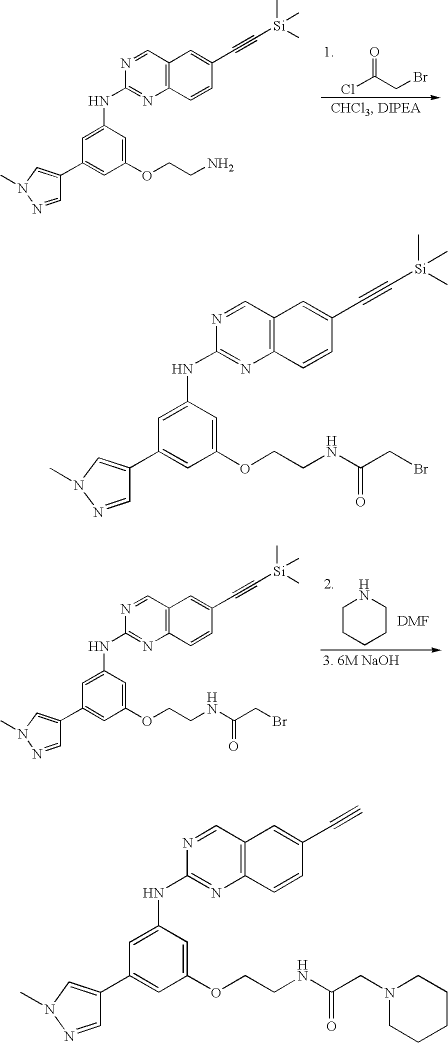

### Step 1: 2-bromo-N-(2-(3-(1-methyl-1H-pyrazol-4-yl)-5-(6-((trimethylsilyl)ethynyl)quinazolin-2-ylamino)phenoxy)ethyl)acetamide

To the reaction mixture of N-(3-(2-aminoethoxy)-5-(1-methyl-1H-pyrazol-4-yl)phenyl)-6-((trimethylsilyl)ethynyl)quinazolin-2-amine (295 mg, 0.646 mmol) in 9 ml of chloroform was added DIPEA (0.394 ml, 2.26 mmol) and cooled to (−5° C.) with stirring. To the above reaction mixture at (−5° C.) add dropwise a solution of 2-bromoacetyl chloride (122.5 mg, 0.775 mmol) in 2 ml of chloroform. This reaction mixture was stirred at (−5° C.) for 20 minutes then allowed to warm to room temperature for 70 minutes or until done by LCMS. The chloroform was concentrated off, 300 ml of ethyl acetate was added, washed with saturated NaHCO₃, water, brine, dried with Na₂SO₄, filtered and concentrated to residue. The crude residue was purified by silica gel column chromatography and concentrated in vaccuo to give 2-bromo-N-(2-(3-(1-methyl-1H-pyrazol-4-yl)-5-(6-((trimethylsilyl)ethynyl)quinazolin-2-ylamino)phenoxy)ethyl)acetamide, (155 mg). ES/MS m/z 577/579 (MH⁺).

### Step 2: N-(2-(3-(1-methyl-1H-pyrazol-4-yl)-5-(6-((trimethylsilyl-ethynyl)quinazolin-2-ylamino)phenoxy)ethyl)-2-piperidin-1-yl)acetamide

To the reaction mixture of 2-bromo-N-(2-(3-(1-methyl-1H-pyrazol-4-yl)-5-(6-((trimethylsilyl)ethynyl)quinazolin-2-ylamino)phenoxy)ethyl)acetamide (10.3 mg, 0.0178 mmol) in 0.4 ml of DMF was added piperidine (6 mg, 0.0712 mmol) and stirred at room temperature for 4.5 hours or until done by LCMS. The crude reaction mixture with product, N-(2-(3-(1-methyl-1H-pyrazol-4-yl)-5-(6-((trimethylsilyl)ethynyl)quinazolin-2-ylamino)phenoxy)ethyl)-2-(piperidin-1-yl)acetamide was use in the next step without purification. ES/MS m/z 582 (MH⁴).

### Step 3: N-(2-(3-(6-ethynylquinazolin-2-ylamino)-5-(1-methyl-1H-pyrazol-4-yl)phenoxy)ethyl-2-piperidin-1-yl)acetamide

To the above crude material, N-(2-(3-(1-methyl-1H-pyrazol-4-yl)-5-(6-((trimethylsilyl)ethynyl)quinazolin-2-ylamino)phenoxy)ethyl)-2-(piperidin-1-yl)acetamide (0.0178 mmol) add 6 M NaOH (0.03 ml, 0.18 mmol) and stir at room temperature for 10 minutes or until done by LCMS. If the reaction is not complete add more 6M NaOH as needed. The crude reaction mixture was purified by the addition of 0.5 ml of DMF, filtered, purified on prep HPLC and lyophilized to give, N-(2-(3-(6-ethynylquinazolin-2-ylamino)-5-(1-methyl-1H-pyrazol-4-yl)phenoxy)ethyl)-2-(piperidin-1-yl)acetamide as TFA salt (6.9 mg). ES/MS m/z 510 (MH⁺).

### Example 523

### (Table 2): N-(2-(3-(6-ethynylquinazolin-2-ylamino)-5-(1-methyl-1H-pyrazol-4-yl)phenoxy)ethyl)-3-fluoropicolinamide

The subject compound was prepared according to the general Scheme below:

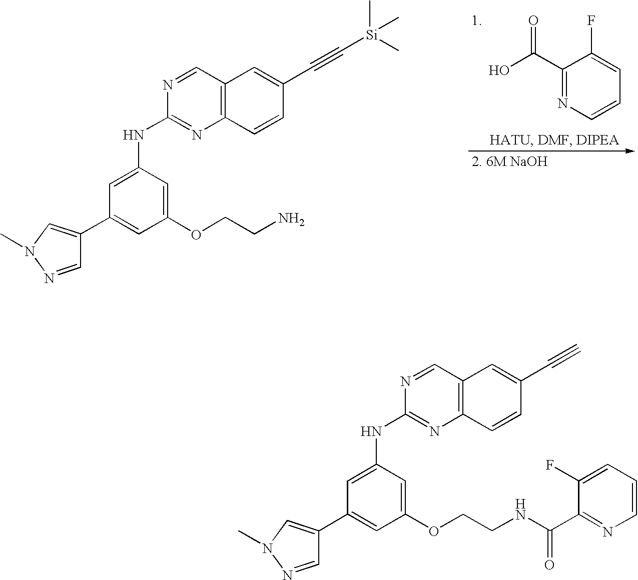

### Step 1: 3-fluoro-N-(2-(3-(1-methyl-1H-pyrazol-4-yl)-5-(6-((trimethylsilyl)ethynyl)quinazolin-2-ylamino)phenoxy)ethyl)picolinamide

Analogous to Example 114, step 1 but using 3-fluoropicolinic acid as starting material. ES/MS m/z 457 (MH⁺).

### Step 2: N-(2-(3-(6-ethynylquinazolin-2-ylamino)-5-(1-methyl-1H-pyrazol-4-yl)phenoxy)ethyl)-3-fluoropicolinamide

To the above crude product from step 1 (0.048 mmol) add 6 M NaOH (0.100 ml, 0.60 mmol) and stir at room temperature for 10 minutes or until done by LCMS. If the reaction is not complete add more 6M NaOH as needed. The crude reaction mixture was purified by the addition of DMF about 0.7 ml, filtered, purified on prep HPLC and lyophilized to give, N-(2-(3-(6-ethynylquinazolin-2-ylamino)-5-(1-methyl-1H-pyrazol-4-yl)phenoxy)ethyl)-3-fluoropicolinamide as TFA salt (6.3 mg). ES/MS m/z 508 (MH⁺).

### Example 536

### (Table 2): 6-ethynyl-N-(3-(1-methyl-1H-pyrazol-4-yl)-5-(pyridin-2-ylmethoxy)phenyl)quinazolin-2-amine

The subject compound was prepared according to the general Scheme, below:

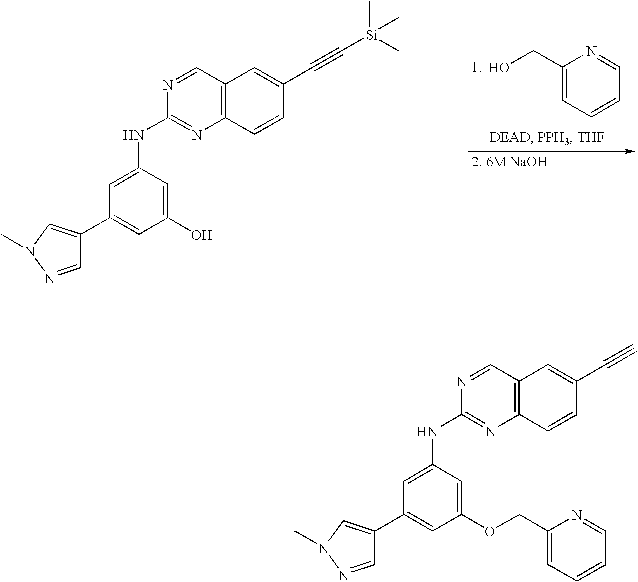

### Step 1: N-(3-(1-methyl-1H-pyrazol-4-yl)-5-(pyridin-2-ylmethoxy)phenyl)-6-((trimethylsilyl)ethynyl)quinazolin-2-amine

To the reaction mixture of 3-(1-methyl-1H-pyrazol-4-yl)-5-(6-((trimethylsilyl)ethynyl)quinazolin-2-ylamino)phenol (24.8 mg, 0.06 mmol) in 0.4 ml of THF add pyridin-2-ylmethanol (16.5, 0.15 mmol), triphenyl phosphine (32 mg, 0.12 mmol) and then DEAD (21 mg, 0.12 mmol) last. The reaction mixture was stirred at room temperature for 20 hours or until done by LCMS. If the reaction is not done add more DEAD (10.5 mg, 0.06 mol) and stir at room temperature 1 to 2 hours. The reaction mixture was concentrated under reduced pressure to give N-(3-(1-methyl-1H-pyrazol-4-yl)-5-(pyridin-2-ylmethoxy)phenyl)-6-((trimethylsilyl)ethynyl)quinazolin-2-amine as a crude residue used in the next step. ES/MS m/z 505 (MH⁺).

### Step 2: 6-ethynyl-N-(3-(1-methyl-1H-pyrazol-4-yl)-5-(pyridin-2-ylmethoxy)phenyl)quinazolin-2-amine

To the crude reaction mixture of N-(3-(1-methyl-1H-pyrazol-4-yl)-5-(pyridin-2-ylmethoxy)phenyl)-6-((trimethylsilyl)ethynyl)quinazolin-2-amine (0.06 mmol) in 1.2 ml of DMF was added 6M NaOH (0.125 ml, 0.75 mmol) and stirred at room temperature for 10 minutes and checked by LCMS. If the de-protection is incomplete add more 6 M NaOH and recheck in 10 minutes. The crude reaction mixture was filtered, purified on prep HPLC and lyophilized to give 6-ethynyl-N-(3-(1-methyl-1H-pyrazol-4-yl)-5-(pyridin-2-ylmethoxy)phenyl)quinazolin-2-amine as TFA salt (5.6 mg). ES/MS m/z 433 (MH⁺).

### Example 543

### (Table 2): (2S,4S)-methyl 4-(3-(6-ethynylquinazolin-2-ylamino)-5-(1-methyl-1H-pyrazol-4-yl)phenoxy)pyrrolidine-2-carboxylate

The subject compound was prepared according to the general Scheme below:

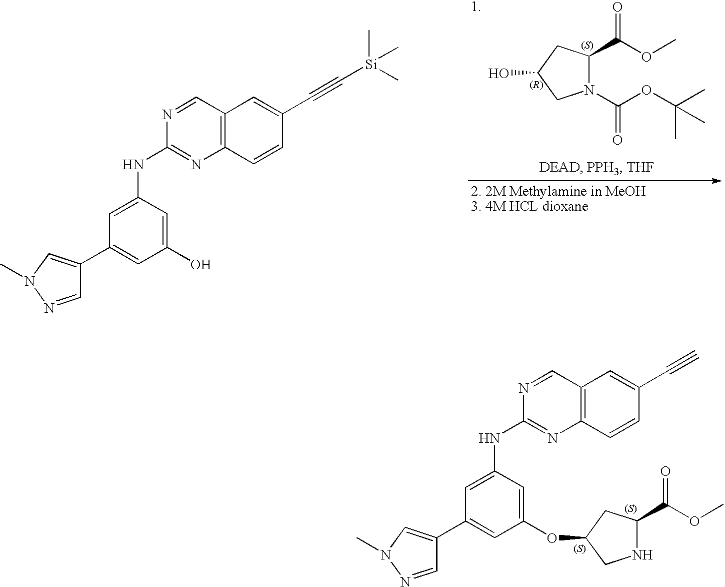

### Step 1: (2S,4S)-1-tert-butyl 2-methyl 4-(3-(1-methyl-1H-pyrazol-4-yl)-5-(6-((trimethylsilyl)ethynyl)quinazolin-2-ylamino)phenoxy)pyrrolidine-1,2-dicarboxylate

Analogous to Example 459, step 4 but using, (2S,4R)-1-tert-butyl 2-methyl 4-hydroxypyrrolidine-1,2-dicarboxylate as the starting material (alcohol). ES/MS m/z 641 (MH⁺).

### Step 2: (2S,4S)-1-tent-butyl 2-methyl 4-(3-(6-ethynylquinazolin-2-ylamino)-5-(1-methyl-1H-pyrazol-4-yl)phenoxy)pyrrolidine-1,2-dicarboxylate

To the reaction mixture of (2S,4S)-1-tert-butyl 2-methyl 4-(3-(1-methyl-1H-pyrazol-4-yl)-5-(6-((trimethylsilyl)ethynyl)quinazolin-2-ylamino)phenoxy)pyrrolidine-1,2-dicarboxylate (38 mg, 0.06 mmol) in 0.375 ml of THF was added 2M methylamine in MeOH (2.0 ml, 4.0 mmol). The reaction mixture was stirred at room temperature for 20 hours or until done by LCMS. The crude reaction mixture was concentrated in vaccuo until dry, to give a crude product used in next step, (2S,4S)-1-tert-butyl 2-methyl 4-(3-(6-ethynylquinazolin-2-ylamino)-5-(1-methyl-1H-pyrazol-4-yl)phenoxy)pyrrolidine-1,2-dicarboxylate. ES/MS m/z 569 (MH⁺).

### Step 3: (2S,4S)-methyl 4-(3-(6-ethynylquinazolin-2-ylamino)-5-(1-methyl-1H-pyrazol-4-yl)phenoxy)pyrrolidine-2-carboxylate

To the reaction mixture of (2S,4S)-1-tert-butyl 2-methyl 4-(3-(6-ethynylquinazolin-2-ylamino)-5-(1-methyl-1H-pyrazol-4-yl)phenoxy)pyrrolidine-1,2-dicarboxylate (0.06 mmol) was added 4M HCl in Dioxane (3.5 ml, 14.0 mmol). The reaction mixture was stirred at room temperature for 1 hours or until done by LCMS. The crude reaction mixture was concentrate, about 1 ml of DMF was added, filtered, purified on prep HPLC and lyophilized to give, (2S,4S)-methyl 4-(3-(6-ethynylquinazolin-2-ylamino)-5-(1-methyl-1H-pyrazol-4-yl)phenoxy)pyrrolidine-2-carboxylate as TFA salt (3.7 mg). ES/MS m/z 469 (MH⁴).

### Example 555

### (Table 2): (2S,4S)-4-(3-(6-ethynylquinazolin-2-ylamino)-5-(1-methyl-1H-pyrazol-4-yl)phenoxy)pyrrolidine-2-carboxylic acid

The subject compound was prepared according to the general Scheme below:

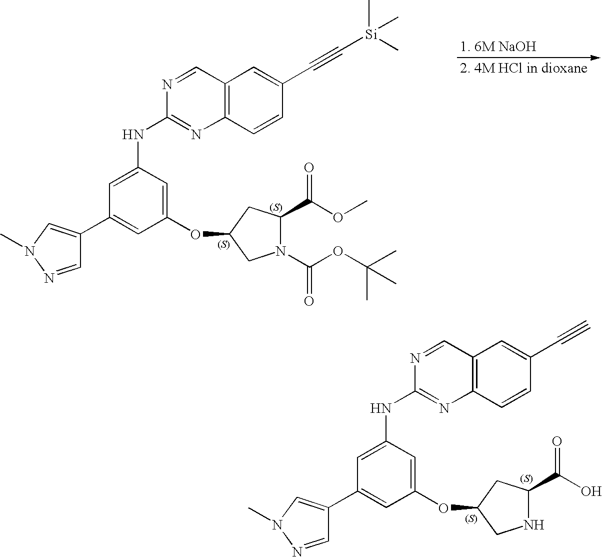

### Step 1: (2S,4S)-1-(tert-butoxycarbonyl)-4-(3-(6-ethynylquinazolin-2-ylamino)-5-(1-methyl-1H-pyrazol-4-yl)phenoxy)pyrrolidine-2-carboxylic acid

To the reaction mixture of (2S,4S)-1-tert-butyl 2-methyl 4-(3-(1-methyl-1H-pyrazol-4-yl)-5-(6-((trimethylsilyl)ethynyl)quinazolin-2-ylamino)phenoxy)pyrrolidine-1,2-dicarboxylate (115 mg, 0.18 mmol) in 0.75 ml of THF was added 6M NaOH (0.9 ml, 5.4 mmol) and 1 ml of methanol. The reaction mixture was stirred at room temperature for 1 hour or until done by LCMS. The crude reaction mixture was concentrated in vaccuo until solid, 2 ml of DMF was added, filtered, purified on prep HPLC and lyophilized to give (2S,4S)-1-(tert-butoxycarbonyl)-4-(3-(6-ethynylquinazolin-2-ylamino)-5-(1-methyl-1H-pyrazol-4-yl)phenoxy)pyrrolidine-2-carboxylic acid as TFA salt (45 mg). ES/MS m/z 555 (MH⁺).

### Step 2: (2S,4S)-4-(3-(6-ethynylquinazolin-2-ylamino)-5-(1-methyl-1H-pyrazol-4-yl)phenoxy)pyrrolidine-2-carboxylic acid

To the reaction mixture of (2S,4S)-1-(tert-butoxycarbonyl)-4-(3-(6-ethynylquinazolin-2-ylamino)-5-(1-methyl-1H-pyrazol-4-yl)phenoxy)pyrrolidine-2-carboxylic acid (15 mg, 0.027 mmol) was added 4M HCl in Dioxane (1.5 ml, 6.0 mmol). The reaction mixture was stirred at room temperature for 1 hour or until done by LCMS. The crude reaction mixture was concentrate, about 1 ml of DMF was added, filtered, purified on prep HPLC and lyophilized to give (2S,4S)-4-(3-(6-ethynylquinazolin-2-ylamino)-5-(1-methyl-1H-pyrazol-4-yl)phenoxy)pyrrolidine-2-carboxylic acid as TFA salt (3.2 mg). ES/MS m/z 455 (MH⁺).

### Example 557

### (Table 2): (2S,4S)-4-(3-(6-ethynylquinazolin-2-ylamino)-5-(1-methyl-1H-pyrazol-4-yl)phenoxy)-N-methylpyrrolidine-2-carboxamide

The subject compound was prepared according to the general Scheme below:

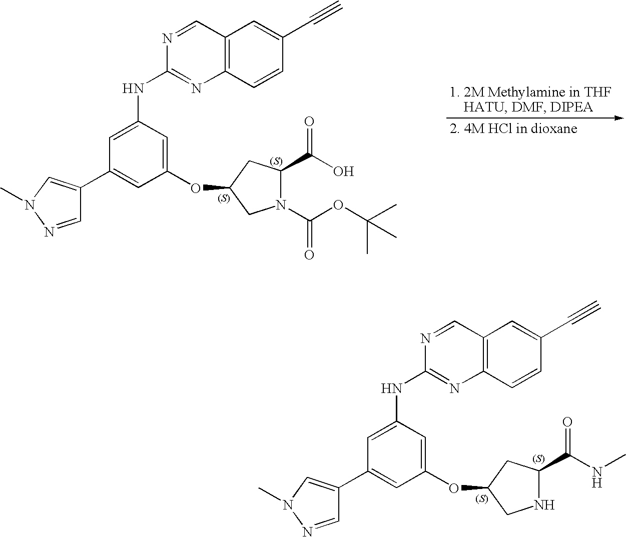

### Step 1: (2S,4S)-tert-butyl 4-(3-(6-ethynylquinazolin-2-ylamino)-5-(1-methyl-1H-pyrazol-4-yl)phenoxy)-2-(methylcarbamoyl)pyrrolidine-1-carboxylate

To the reaction mixture of (2S,4S)-1-(tert-butoxycarbonyl)-4-(3-(6-ethynylquinazolin-2-ylamino)-5-(1-methyl-1H-pyrazol-4-yl)phenoxy)pyrrolidine-2-carboxylic acid (15 mg, 0.027 mmol) in 0.5 ml of DMF add HATU (31 mg, 0.081 mmol), DIPEA (0.014 ml, 0.081 mmol) and stir at room temperature for about 3-5 minutes. To the above reaction mixture add 2M methylamine in THF (0.081 ml, 0.162 mmol) and was stirred at room temperature for 20 hours or until done by LCMS. The crude reaction mixture with product, (2S,4S)-tert-butyl 4-(3-(6-ethynylquinazolin-2-ylamino)-5-(1-methyl-1H-pyrazol-4-yl)phenoxy)-2-(methylcarbamoyl)pyrrolidine-1-carboxylate was use in the next step without purification. ES/MS m/z 568 (MH⁺).

### Step 2: (2S,4S)-4-(3-(6-ethynylquinazolin-2-ylamino)-5-(1-methyl-1H-pyrazol-4-yl)phenoxy)-N-methylpyrrolidine-2-carboxamide

To the reaction mixture of (2S,4S)-tert-butyl 4-(3-(6-ethynylquinazolin-2-ylamino)-5-(1-methyl-1H-pyrazol-4-yl)phenoxy)-2-(methylcarbamoyl)pyrrolidine-1-carboxylate (0.027 mmol) was added 4M HCl in Dioxane (2.0 ml, 8.0 mmol). The reaction mixture was stirred at room temperature for 1 hours or until done by LCMS. The crude reaction mixture was concentrate, about 1 ml of DMF was added, filtered, purified on prep HPLC and lyophilized to give (2S,4S)-4-(3-(6-ethynylquinazolin-2-ylamino)-5-(1-methyl-1H-pyrazol-4-yl)phenoxy)-N-methylpyrrolidine-2-carboxamide as TFA salt (5.0 mg). ES/MS m/z 468 (MH⁺).

### Example 552

### (Table 2): 6-ethynyl-N-(3-(1-methyl-1H-pyrazol-4-yl)-5-(2-(tetrahydro-2H-pyran-4-ylamino)ethoxy)phenyl)quinazolin-2-amine

The subject compound was prepared according to the general Scheme below:

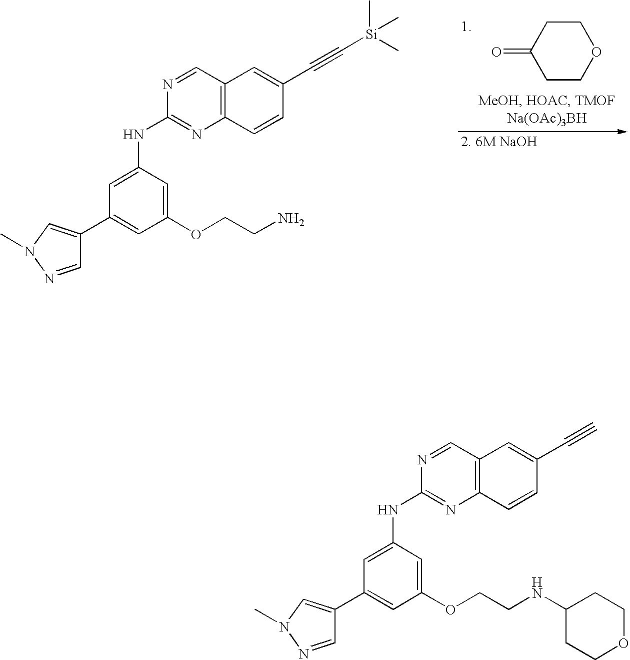

### Step 1: N-(3-(1-methyl-1H-pyrazol-4-yl)-5-(2-(tetrahydro-2H-pyran-4-ylamino)ethoxy)phenyl)-6-((trimethylsilyl)ethynyl)quinazolin-2-amine

To the reaction mixture of N-(3-(2-aminoethoxy)-5-(1-methyl-1H-pyrazol-4-yl)phenyl)-6-((trimethylsilyl)ethynyl)quinazolin-2-amine (41 mg, 0.09 mmol) in 0.75 ml of methanol add acetic acid (0.162 ml, 2.7 mmol), dihydro-2H-pyran-4(3H)-one (90 mg, 0.9 mmol) and trimethylorthoformate (TMOF) (57 mg, 0.54 mmol). The reaction mixture was stirred at room temperature for about 4 hours. To this reaction solution was added sodium triacetoxy borohydride (76 mg, 0.36 mmol) and stirred at room temperature 20 hours. To the crude reaction mixture was added more sodium triacetoxy borohydride (38 mg, 0.18 mmol) and stirred at room temperature for another 26 hours. The crude reaction mixture with product, N-(3-(1-methyl-1H-pyrazol-4-yl)-5-(2-(tetrahydro-2H-pyran-4-ylamino)ethoxy)phenyl)-6-((trimethylsilyl)ethynyl)quinazolin-2-amine was concentrated to solid and used in the next step without purification. ES/MS m/z 541 (MH⁺).

### Step 2: 6-ethynyl-N-(3-(1-methyl-1H-pyrazol-4-yl)-5-(2-(tetrahydro-2H-pyran-4-ylamino)ethoxy)phenyl)quinazolin-2-amine

To the crude reaction mixture of N-(3-(1-methyl-1H-pyrazol-4-yl)-5-(2-(tetrahydro-2H-pyran-4-ylamino)ethoxy)phenyl)-6-((trimethylsilyl)ethynyl)quinazolin-2-amine (0.09 mmol) in 2 ml of methanol was added 6M NaOH (2.0 ml, 12.0 mmol) and stirred at room temperature for 10 minutes and checked by LCMS. If the de-protection is incomplete add more 6 M NaOH and recheck in 10 minutes. To the crude reaction add 100 ml of ethyl acetate and wash with water (2×), saturated NaCl, dried Na₂SO₄, filtered and concentrated to residue. To the crude residue add DMF, filter, purified on prep HPLC and lyophilized to give 6-ethynyl-N-(3-(1-methyl-1H-pyrazol-4-yl)-5-(2-(tetrahydro-2H-pyran-4-ylamino)ethoxy)phenyl)quinazolin-2-amine as TFA salt (2.0 mg). ES/MS m/z 469 (MH⁺).

### Example 530

### (Table 2): 6-(2-(3-(6-ethynylquinazolin-2-ylamino)-5-(1-m ethyl-1H-pyrazol-4-yl)phenoxy)ethylamino)nicotinonitrile

The subject compound was prepared according to the general Scheme below:

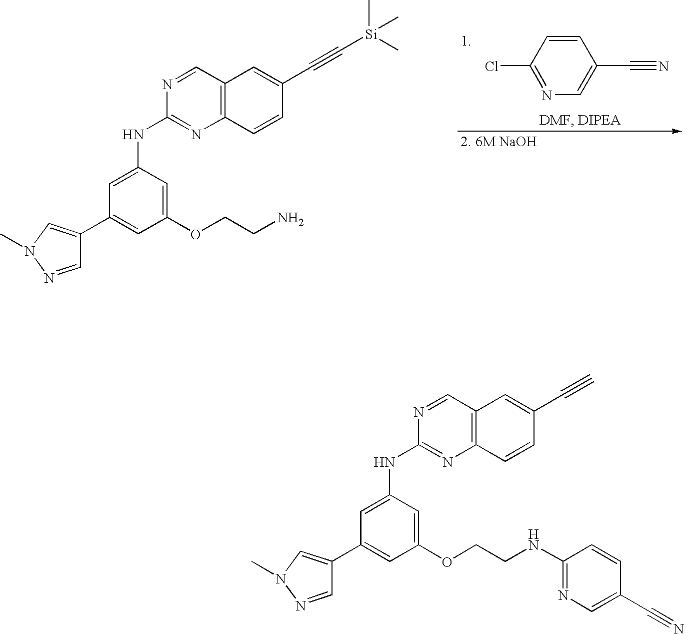

### Step 1: 6-(2-(3-(1-methyl-1H-pyrazol-4-yl-5-(6-((trimethylsilyl)ethynyl)quinazolin-2-ylamino)phenoxy)ethylamino)nicotinonitrile

To the reaction mixture of N-(3-(2-aminoethoxy)-5-(1-methyl-1H-pyrazol-4-yl)phenyl)-6-((trimethylsilyl)ethynyl)quinazolin-2-amine (26.7 mg, 0.0479 mmol) in DMF add 6-chloronicotinonitrile (13.2 mg, 0.096) and DIPEA (0.025 ml, 0.144 mmol). This reaction mixture was stirred at 105° C. for 20 hours. The crude reaction mixture with product, 6-(2-(3-(1-methyl-1H-pyrazol-4-yl)-5-(6-((trimethylsilyl)ethynyl)quinazolin-2-ylamino)phenoxy)ethylamino)nicotinonitrile was used in the next step without purification. ES/MS m/z 559 (MH⁺).

### Step 2: 6-(2-(3-(6-ethynylquinazolin-2-ylamino)-5-(1-methyl-1H-pyrazol-4-yl)phenoxy)ethylamino)nicotinonitrile

To the crude reaction mixture of, 6-(2-(3-(1-methyl-1H-pyrazol-4-yl)-5-(6-((trimethylsilyl)ethynyl)quinazolin-2-ylamino)phenoxy)ethylamino)nicotinonitrile (0.0479 mmol) was added 6M NaOH (0.08 ml, 0.48 mmol) and stirred at room temperature for 10 minutes and checked by LCMS. If the de-protection is incomplete add more 6 M NaOH and recheck in 10 minutes. To the crude reaction mixture add DMF, filter, purified on prep HPLC and lyophilized to give, 6-(2-(3-(6-ethynylquinazolin-2-ylamino)-5-(1-methyl-1H-pyrazol-4-yl)phenoxy)ethylamino)nicotinonitrile as TFA salt (1.7 mg). ES/MS m/z 487 (MH⁺).

### Example 593

### (Table 2): 4-(6-bromo-8-isopropoxyquinazolin-2-ylamino)benzenesulfonamide and 4-(6-ethynyl-8-isopropoxyquinazolin-2-ylamino) benzenesulfonamide

The subject compound was prepared according to the general Scheme below

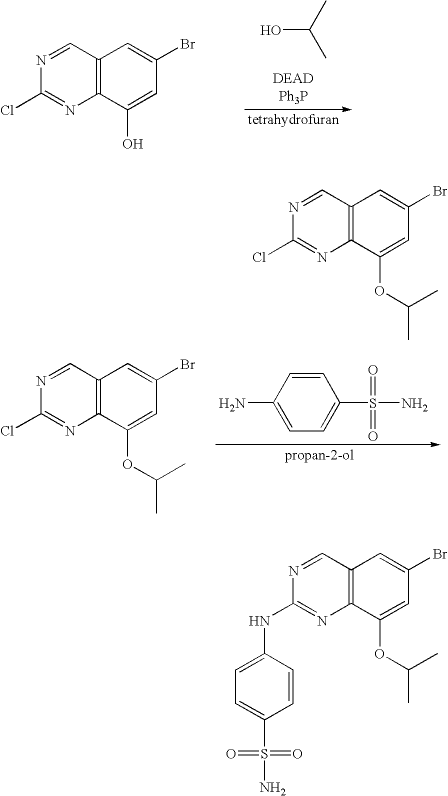

### Step 1: Preparation of 6-bromo-2-chloro-8-isopropoxyquinazoline

To the reaction solution of triphenyl phosphine (1.91 g, 7.3 mmole), isopropyl alcohol (782 ul, 10.22 mmole) and diethyl azodicarboxylate (1.15 ml, 7.3 mmole) in 18 ml of tetrahydrofuran, 6-bromo-2-chloroquinazolin-8-ol (928 mg, 3.64 mmole) in 20 ml of tetrahydrofuran was added. The resulting solution was stirred at 65° c. for 24 hours or until done by LCMS. The reaction solution was concentrated under reduced pressure to give brown glue as a crude product that was purified by column chromatography over silica gel with ethyl acetate:hexane (15:85) to give 587 mg of 6-bromo-2-chloro-8-isopropoxyquinazoline as yellow solid. ES/MS m/z 300.9/302.9 (MH⁺).

### Step 2: Preparation of 4-(6-bromo-8-isopropoxyquinazolin-2-ylamino) benzenesulfonamide

To the reaction mixture of 6-bromo-2-chloro-8-isopropoxyquinazoline (45 mg, 150 umole) in 0.8 ml of isopropyl alcohol, 4-aminobenzenesulfonamide (51 mg, 300 mmole) was added and stirred at 82° c. for 20 hours or until done by LCMS. The crude reaction mixture was filtered, purified on prep HPLC and lyophilized to give 4-(6-bromo-8-isopropoxyquinazolin-2-ylamino)benzenesulfonamide as TFA salt (57 mg). ES/MS m/z 437.0/439.0 (MH⁺).

The ethynyl compound was prepared according to the general Scheme below

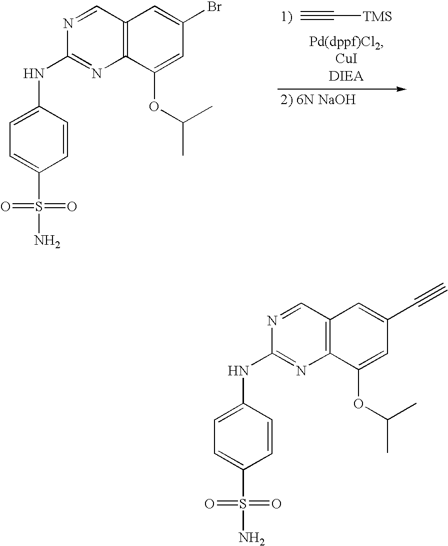

### Step 1: Preparation of 4-(6-ethynyl-8-isopropoxyquinazolin-2-ylamino) benzenesulfonamide

To the reaction mixture of 4-(6-bromo-8-isopropoxyquinazolin-2-ylamino) benzenesulfonamide (57 mg, 130 umole), Pd(dppf)Cl₂ (11 mg, 13 umole), CuI (5 mg, 26 umole) and DIPEA (80 ul, 460 umole) in 0.8 ml of N,N-dimethylformamide, trimethylsilylacetylene (55 ul, 390 umole) was added. The reaction mixture was stirred at 55° c. for 2 hour or until done by LCMS. The crude product was deprotected with 50 ul of 6N NaOH solution for 5 minutes and then neutralized with 60 ul of acetic acid. The crude reaction mixture was filtered, purified on prep HPLC and lyophilized to give 4-(6-ethynyl-8-isopropoxyquinazolin-2-ylamino)benzenesulfonamide as TFA salt (22 mg). ES/MS m/z 383.1 (MH⁺).

### Example 606

### (Table 2): (R)-2-(dimethylamino)-N-(1-(3-(6-ethynylquinazolin-2-ylamino)-5-(1-methyl-1H-pyrazol-4-yl)phenoxy)propan-2-yl)acetamide

### 3-(1-methyl-1H-pyrazol-4-yl)-5-nitrophenol

The subject compound was prepared according to the general Scheme below:

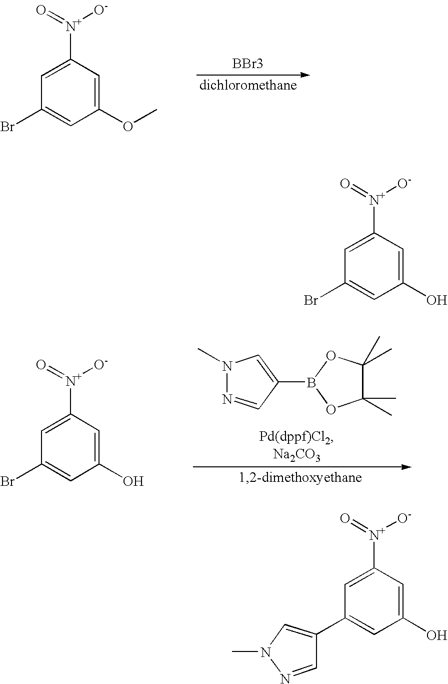

### Step 1: Preparation of 3-bromo-5-nitrophenol

To the solution of 1-bromo-3-methoxy-5-nitrobenzene (13.5 g, 58.2 mmole) in 50 ml of dichloromethane was added the solution of 1 M of boron tribromide (163 ml, 163 mmole) in dichloromethane slowly over 10 minutes at 0° c. The reaction mixture was stirred at 0° c. for 20 minutes and then at room temperature for 48 hour. The deprotection of the methyl ether went to completion and was monitored by LC/MS. Removal of all solvent in vaccuo, followed by quenching with water and diluted NaHCO₃ solution at 0° c., and extraction of aqueous phase with ethyl acetate and drying of combined organic extracts over Na₂SO₄ and subsequent removal of ethyl acetate in vaccuo yielded the desired product that was dried under vacuum to give 12.3 g of 3-bromo-5-nitrophenol as purple solid.

### Step 2: Preparation of 3-(1-methyl-1H-pyrazol-4-yl)-5-nitrophenol

To the reaction mixture of 3-bromo-5-nitrophenol (2.2 g, 10 mmole) and 1-methyl-4-(4,4,5,5-tetramethyl-1,3,2-dioxaborolan-2-yl)-1H-pyrazole (2.7 g, 13 mmole) in 37.5 ml of 1,2-dimethoxyethane, Pd(dppf)Cl₂ (660 mg, 0.8 mmole) and aq. 2M Na₂CO_{3 (}12.5 ml, 25 mmole) were added. The reaction mixture was stirred at 85° c. for 24 hours or until done by LC. The reaction mixture was diluted with 250 ml of acetone and filtered through celite and washed with a 1:1 solution of ethyl acetate and methanol. The combined organic filtrate was evaporated in vaccuo to give a brown solid (4 g) that was purified by silica gel column plug eluting with ethyl acetate to give 3-(1-methyl-1H-pyrazol-4-yl)-5-nitrophenol as yellow brown powder (1.87 g). ES/MS m/z 220.0 (MH⁺).

### (S)-tert-butyl 1-(3-amino-5-(1-methyl-1H-pyrazol-4-yl)phenoxy)propan-2-ylcarbamate

The subject compound was prepared according to the general Scheme below:

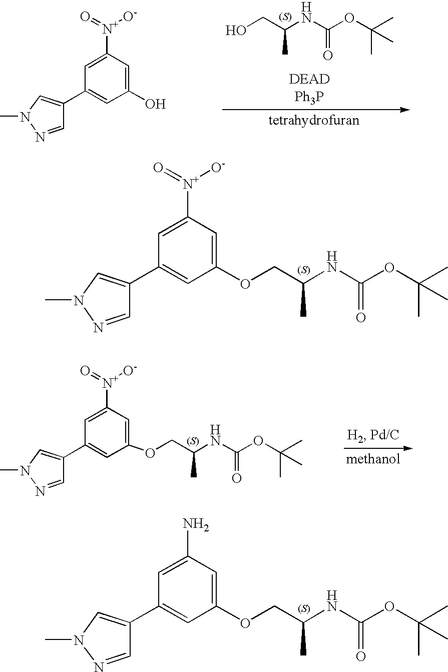

### Step 1: Preparation of (S)-tent-butyl 1-(3-(1-methyl-1H-pyrazol-4-yl)-5-nitrophenoxy)propan-2-ylcarbamate

To the reaction solution of 3-(1-methyl-1H-pyrazol-4-yl)-5-nitrophenol (700 mg, 3.2 mmole) and triphenyl phosphine (1.26 g, 4.84 mmole) in 26 ml of tetrahydrofuran, (S)-tert-butyl 1-hydroxypropan-2-ylcarbamate (848 mg, 4.84 mmole) and diethyl azodicarboxylate (860 ul, 5.46 mmole) were added. The resulting solution was stirred at 65° c. for 20 hours or until done by LCMS. The reaction solution was concentrated under reduced pressure to give dark brown glue (4 g) as a crude product that was purified by column chromatography over silica gel with ethyl acetate:hexane (60:40) to give 1.08 g of (S)-tert-butyl 1-(3-(1-methyl-1H-pyrazol-4-yl)-5-nitrophenoxy)propan-2-ylcarbamate as yellow solid. ES/MS m/z 377.2 (MH⁺).

### Step 2: Preparation of (S)-tert-butyl 1-(3-amino-5-(1-methyl-1H-pyrazol-4-yl)phenoxy) propan-2-ylcarbamate

To the starting crude material (S)-tert-butyl 1-(3-(1-methyl-1H-pyrazol-4-yl)-5-nitrophenoxy)propan-2-ylcarbamate (1.09 g, 2.89 mmole) was added 10% Pd on Carbon (1.02 g, 0.96 mmole, 30% by wt.) under argon. Under argon add 11 ml of methanol with a syringe carefully. To this reaction mixture was added a balloon of hydrogen and was evacuated and refilled 6 times. The reaction mixture was stirred at room temperature for 22 hours or until done by LC. To the reaction mixture add ethyl acetate and under argon filtered through celite and washed with a 1:1 solution of ethyl acetate and methanol. The filtrate was concentrated under reduced pressure to give (S)-tert-butyl 1-(3-amino-5-(1-methyl-1H-pyrazol-4-yl)phenoxy)propan-2-ylcarbamate as powder (945 mg). ES/MS m/z 347.1 (MH⁺).

### (S)—N-(3-(2-aminopropoxy)-5-(1-methyl-1H-pyrazol-4-yl)phenyl)-6-((trimethylsilyl)ethynyl)quinazolin-2-amine

The subject compound was prepared according to the general Scheme below

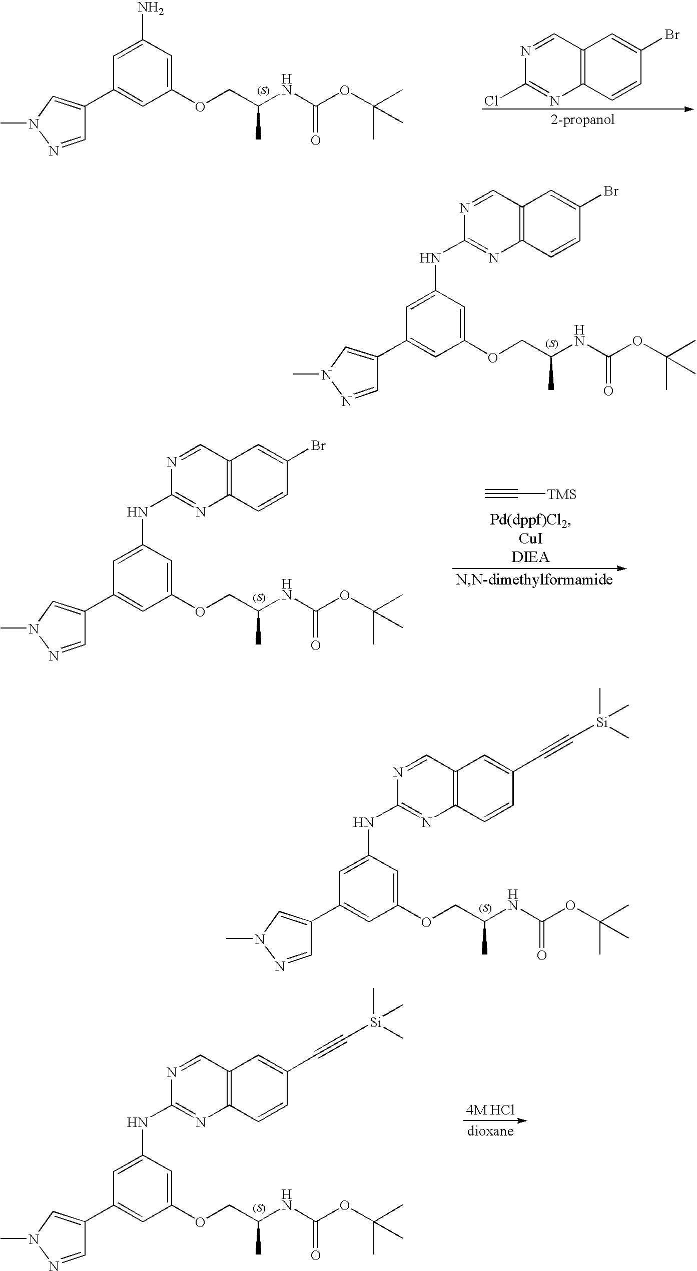

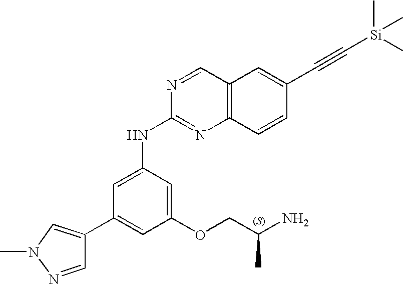

### Step 1: Preparation of (S)-tert-butyl 1-(3-(6-bromoquinazolin-2-ylamino)-5-(1-methyl-1H-pyrazol-4-yl)phenoxy)propan-2-ylcarbamate

To the reaction mixture give (S)-tert-butyl 1-(3-amino-5-(1-methyl-1H-pyrazol-4-yl)phenoxy)propan-2-ylcarbamate (945 mg, 2.73 mmole) in 25 ml of isopropyl alcohol in a glass bomb was added 6-bromo-2-chloroquinazoline (665 mg, 2.73 mmole) and sealed. The reaction solution was stirred at 95° c. for 22 hours or until done by LCMS. The reaction solution was concentrated under reduced pressure to give a reddish brown oil that was then diluted with 160 ml of ethyl acetate. The organic phase was washed with saturated NaHCO₃ solution (2×60 ml), water (30 ml) and brine (50 ml), then dried over MgSO₄ and evaporated in vacuo to give a reddish brown solid. The crude solid was purified by column chromatography over silica gel with ethyl acetate:hexane (70:30) to give (S)-tert-butyl 1-(3-(6-bromoquinazolin-2-ylamino)-5-(1-methyl-1H-pyrazol-4-yl)phenoxy)propan-2-ylcarbamate (131 mg) as yellow solid. ES/MS m/z 553.1/555.1

(MH⁺).

### Step 2: Preparation of (S)-tert-butyl 1-(3-(1-methyl-1H-pyrazol-4-yl)-5-(6-((trimethylsilyl)ethynyl)quinazolin-2-ylamino)phenoxy)propan-2-ylcarbamate

To the reaction mixture of (S)-tert-butyl 1-(3-(6-bromoquinazolin-2-ylamino)-5-(1-methyl-1H-pyrazol-4-yl)phenoxy)propan-2-ylcarbamate (131 mg, 237 umole), Pd(dppf)Cl₂ (20 mg, 24 umole), CuI (10 mg, 47.4 umole) and DIPEA (200 ul, 1.15 mmole) in 2 ml of N,N-dimethylformamide, trimethylsilylacetylene (99 ul, 710 umole) was added. The reaction mixture was stirred at 60° c. for 2 hour. The crude reaction mixture was partitioned between 80 ml of ethyl acetate and 20 ml of saturated NaHCO₃ solution. The organic layer was washed with water (30 ml) and brine (50 ml), then dried over Na₂SO₄ and evaporated in vacuo to give a brown solid that was purified by column chromatography over silica gel with ethyl acetate:hexane (50:50) to give (S)-tert-butyl 1-(3-(1-methyl-1H-pyrazol-4-yl)-5-(6-((trimethyl silyl)ethynyl)quinazolin-2-ylamino)phenoxy)propan-2-ylcarbamate (73 mg) as yellow solid. ES/MS m/z 571.3 (MH⁺).

### Step 3: Preparation of (S)—N-(3-(2-aminopropoxy)-5-(1-methyl-1H-pyrazol-4-yl)phenyl)-6-((trimethylsilyl-ethynyl)quinazolin-2-amine

To the reaction mixture (S)-tert-butyl 1-(3-(1-methyl-1H-pyrazol-4-yl)-5-(6-((trimethylsilyl)ethynyl)quinazolin-2-ylamino)phenoxy)propan-2-ylcarbamate (73 mg, 0.13 mmole) add excess 4 M HCl in dioxane (4 ml, 16 mmole). This reaction mixture was stirred at room temperature for 1 hour or until done by LCMS and concentrated in vaccuo to give a yellow solid that was dissolved with 60 ml of ethyl acetate. The organic phase was washed with 1N NaOH solution (10 ml), water (10 ml) and brine solution (20 ml), then dried over Na₂SO₄ and evaporated in vacuo to give (S)—N-(3-(2-aminopropoxy)-5-(1-methyl-1H-pyrazol-4-yl)phenyl)-6-((trimethylsilyl)ethynyl)quinazolin-2-amine as a yellow brown powder (60 mg). ES/MS m/z 471.2 (MH⁺).

### (R)-tert-butyl 1-(3-amino-5-(1-methyl-1H-pyrazol-4-yl)phenoxy) propan-2-ylcarbamate

The subject compound was prepared according to the general Scheme below:

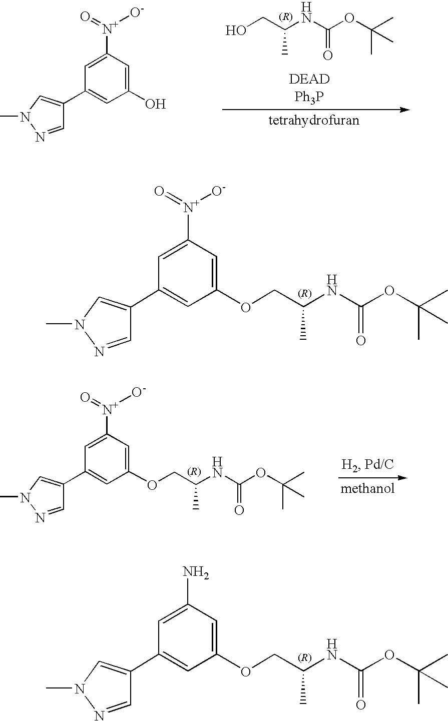

### Step 1: Preparation of (R)-tert-butyl 1-(3-(1-methyl-1H-pyrazol-4-yl)-5-nitrophenoxy)propan-2-ylcarbamate

To the reaction solution of 3-(1-methyl-1H-pyrazol-4-yl)-5-nitrophenol (700 mg, 3.2 mmole) and triphenyl phosphine (1.26 g, 4.84 mmole) in 26 ml of tetrahydrofuran, (R)-tert-butyl 1-hydroxypropan-2-ylcarbamate (848 mg, 4.84 mmole) and diethyl azodicarboxylate (790 ul, 5 mmole) were added. The resulting solution was stirred at 65° c. for 20 hours or until done by LCMS. The reaction solution was concentrated under reduced pressure to give brown glue (4 g) as a crude product that was purified by column chromatography over silica gel with ethyl acetate:hexane (60:40) to give 1.03 g of (R)-tert-butyl 1-(3-(1-methyl-1H-pyrazol-4-yl)-5-nitrophenoxy)propan-2-ylcarbamate as yellow glue. ES/MS m/z 377.1 (MH⁴).

### Step 2: Preparation of (R)-tert-butyl 1-(3-amino-5-(1-methyl-1H-pyrazol-4-yl)phenoxy)propan-2-ylcarbamate

To the starting crude material (R)-tert-butyl 1-(3-(1-methyl-1H-pyrazol-4-yl)-5-nitrophenoxy)propan-2-ylcarbamate (1.03 g, 2.73 mmole) was added 10% Pd on Carbon (968 mg, 0.91 mmole, 30% by wt.) under argon. Under argon add 11 ml of methanol with a syringe carefully. To this reaction mixture was added a balloon of hydrogen and was evacuated and refilled 6 times. The reaction mixture was stirred at room temperature for 22 hours or until done by LC. To the reaction mixture add ethyl acetate and under argon filtered through celite and washed with a 1:1 solution of ethyl acetate and methanol. The filtrate was concentrated under reduced pressure to give (R)-tert-butyl 1-(3-amino-5-(1-methyl-1H-pyrazol-4-yl)phenoxy)propan-2-ylcarbamate as powder (905 mg). ES/MS m/z 347.2 (MH⁺).

### (R)—N-(3-(2-aminopropoxy)-5-(1-methyl-1H-pyrazol-4-yl)phenyl)-6-((trimethylsilyl)ethynyl)quinazolin-2-amine

The subject compound was prepared according to the general Scheme below

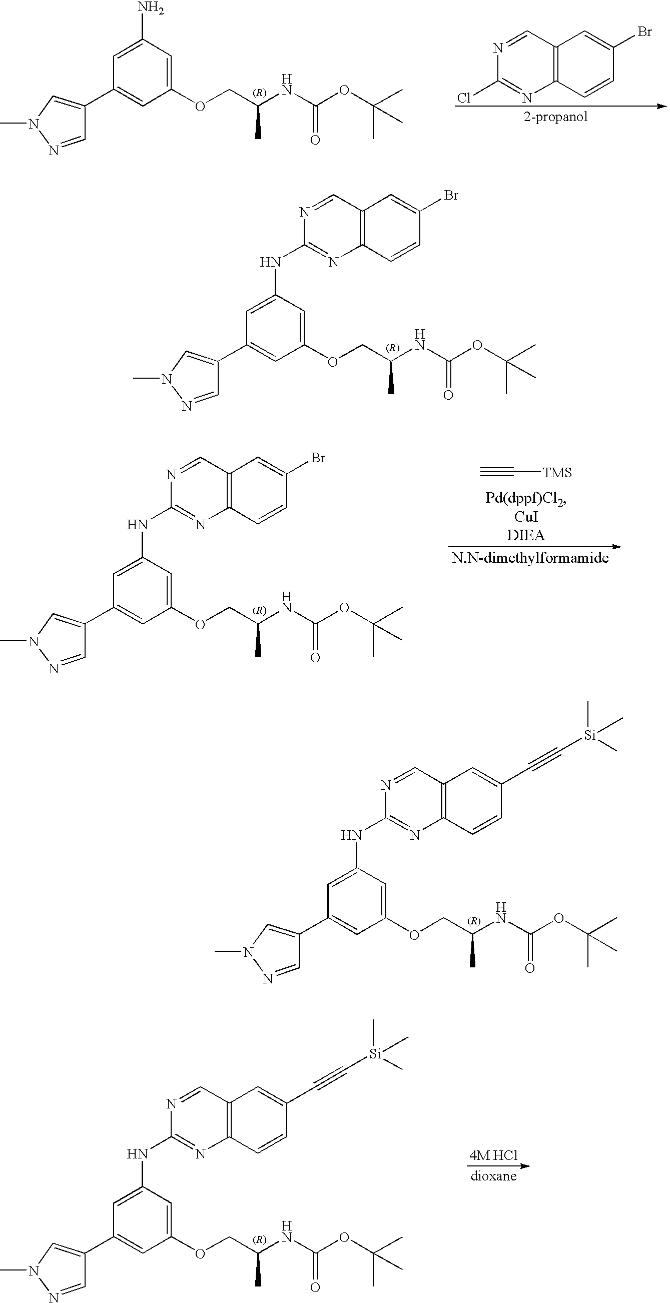

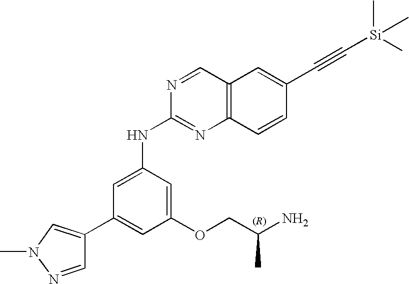

### Step 1: Preparation of (R)-tert-butyl 1-(3-(6-bromoquinazolin-2-ylamino)-5-(1-methyl-1H-pyrazol-4-yl)phenoxy)propan-2-ylcarbamate

To the reaction mixture of (R)-tert-butyl 1-(3-amino-5-(1-methyl-1H-pyrazol-4-yl)phenoxy)propan-2-ylcarbamate (905 mg, 2.35 mmole) and 6-bromo-2-chloroquinazoline (572 mg, 2.35 mmole) in 11 ml of dioxane in a glass bomb, acetic acid (350 ul) was added and then sealed. The reaction solution was stirred at 92° c. for 22 hours or until done by LCMS. The reaction solution was concentrated under reduced pressure to give a brown solid that was then diluted with 180 ml of ethyl acetate. The organic phase was washed with saturated NaHCO₃ solution (2×60 ml), water (30 ml) and brine (50 ml), then dried over Na₂SO₄ and evaporated in vacuo to give a brown solid. The crude solid was purified by column chromatography over silica gel with ethyl acetate:hexane (70:30) to give (R)-tert-butyl 1-(3-(6-bromoquinazolin-2-ylamino)-5-(1-methyl-1H-pyrazol-4-yl)phenoxy)propan-2-ylcarbamate (405 mg) as yellow solid. ES/MS m/z 553.1/555.1 (MH⁺).

### Step 2: Preparation of (R)-tert-butyl 1-(3-(1-methyl-1H-pyrazol-4-yl)-5-(6-((trimethylsilyl)ethynyl)quinazolin-2-ylamino)phenoxy)propan-2-ylcarbamate

To the reaction mixture of (R)-tert-butyl 1-(3-(6-bromoquinazolin-2-ylamino)-5-(1-methyl-1H-pyrazol-4-yl)phenoxy)propan-2-ylcarbamate (408 mg, 737 umole), Pd(dppf)Cl₂ (90 mg, 111 umole), CuI (43 mg, 222 umole) and DIPEA (500 ul, 2.87 mmole) in 5 ml of N,N-dimethylformamide, trimethylsilylacetylene (307 ul, 2.21 mmole) was added. The reaction mixture was stirred at 80° c. for 1.5 hour. The crude reaction mixture was partitioned between 100 ml of ethyl acetate and 40 ml of saturated NaHCO₃ solution. The organic layer was washed with water (30 ml) and brine (50 ml), then dried over Na₂SO₄ and evaporated in vacuo to give a brown solid that was purified by column chromatography over silica gel with ethyl acetate:hexane (60:40) to give (R)-tert-butyl 1-(3-(1-methyl-1H-pyrazol-4-yl)-5-(6-((trimethylsilyl)ethynyl)quinazolin-2-ylamino) phenoxy)propan-2-ylcarbamate (370 mg) as yellow solid . ES/MS m/z 571.3 (MH⁺).

### Step 3: Preparation of (R)—N-(3-(2-aminopropoxy)-5-(1-methyl-1H-pyrazol-4-yl)phenyl)-6-((trimethylsilyl)ethynyl)quinazolin-2-amine

To the reaction mixture (R)-tert-butyl 1-(3-(1-methyl-1H-pyrazol-4-yl)-5-(6-((trimethylsilyl)ethynyl)quinazolin-2-ylamino)phenoxy)propan-2-ylcarbamate (370 mg, 0.65 mmole) add excess 4 M HCl in dioxane (16 ml, 64 mmole). This reaction mixture was stirred at room temperature for 1 hour or until done by LCMS and concentrated in vaccuo to give a yellow solid that was dissolved with 100 ml of ethyl acetate. The organic phase was washed with 1N NaOH solution (20 ml), water (30 ml) and brine solution (40 ml), then dried over Na₂SO₄ and evaporated in vacuo to give (R)—N-(3-(2-aminopropoxy)-5-(1-methyl-1H-pyrazol-4-yl)phenyl)-6-((trimethylsilyl)ethynyl) quinazolin-2-amine as a yellow brown powder (304 mg). ES/MS m/z 471.2 (MH⁺).

### (R)-2-(dimethylamino)-N-(1-(3-(6-ethynylquinazolin-2-ylamino)-5-(1-methyl-1H-pyrazol-4-yl)phenoxy)propan-2-yl)acetamide

The subject compound was prepared according to the general Scheme below

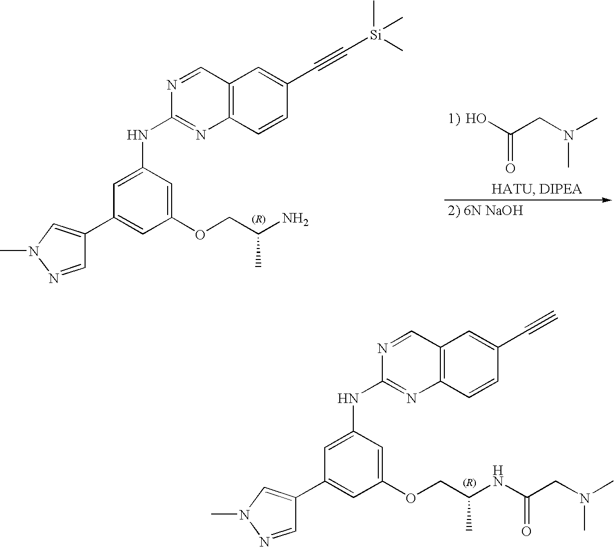

### Step 1: Preparation of (R)-2-(dimethylamino)-N-(1-(3-(6-ethynylquinazolin-2-ylamino)-5-(1-methyl-1H-pyrazol-4-yl)phenoxy)propan-2-yl)acetamide

To the reaction mixture of 2-(dimethylamino)acetic acid (11 mg, 100 umole) in 0.7 ml of N,N-dimethylformamide add HATU (38 mg, 100 umole), DIPEA (45 ul, 250 umole) and stir at room temperature for about 3 minutes. To the above reaction mixture add (R)—N-(3-(2-aminopropoxy)-5-(1-methyl-1H-pyrazol-4-yl)phenyl)-6-((trimethylsilyl)ethynyl) quinazolin-2-amine (19 mg, 32 umole) and stir at room temperature for 1 hours or until done by LCMS. The crude product was deprotected with 60 ul of 6N NaOH solution for 5 minutes and then neutralized with 60 ul of acetic acid. The crude reaction mixture was filtered, purified on prep HPLC and lyophilized to give (R)-2-(dimethylamino)-N-(1-(3-(6-ethynylquinazolin-2-ylamino)-5-(1-methyl-1H-pyrazol-4-yl)phenoxy)propan-2-yl)acetamide as TFA salt (10.3 mg). ES/MS m/z 483.2 (MH⁺).

### Example 607

### (Table 2): (R)—N-(3-(2-(dimethylamino)propoxy)-5-(1-methyl-1H-pyrazol-4-yl)phenyl)-6-ethynylquinazolin-2-amine

The subject compound was prepared according to the general Scheme below

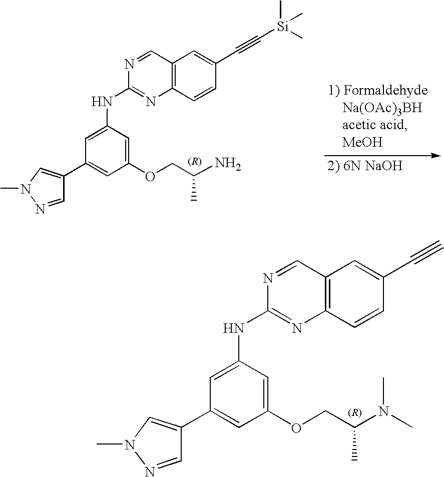

### Step 1: Preparation of (R)—N-(3-(2-(dimethylamino)propoxy)-5-(1-methyl-1H-pyrazol-4-yl)phenyl)-6-ethynylquinazolin-2-amine

To the reaction mixture of (R)—N-(3-(2-aminopropoxy)-5-(1-methyl-1H-pyrazol-4-yl)phenyl)-6-((trimethylsilyl)ethynyl)quinazolin-2-amine (18.5 mg, 32 umole) in 0.6 ml of methanol, add acetic acid (36 ul, 600 umole) and 37% formaldehyde solution in water (26 ul, 300 umole). The reaction mixture was stirred at room temperature for about 30 minutes. To this reaction solution was added sodium triacetoxy borohydride (51 mg, 240 umole) and stirred at room temperature 1-2 hours or until done by LCMS. The crude product was deprotected with 60 ul of 6N NaOH solution for 5 minutes and then neutralized with 60 ul of acetic acid. The crude reaction mixture was concentrated, 1 ml of DMF added, filtered, purified on prep HPLC and lyophilized to give (R)—N-(3-(2-(dimethylamino)propoxy)-5-(1-methyl-1H-pyrazol-4-yl)phenyl)-6-ethynylquinazolin-2-amine as TFA salt (8 mg). ES/MS m/z 426.2 (MH⁺).

### Example 618

### (Table 2): (R)—N-(1-(3-(6-ethynylquinazolin-2-ylamino)-5-(1-methyl-1H-pyrazol-4-yl)phenoxy)propan-2-yl)acetamide

The subject compound was prepared according to the general Scheme below

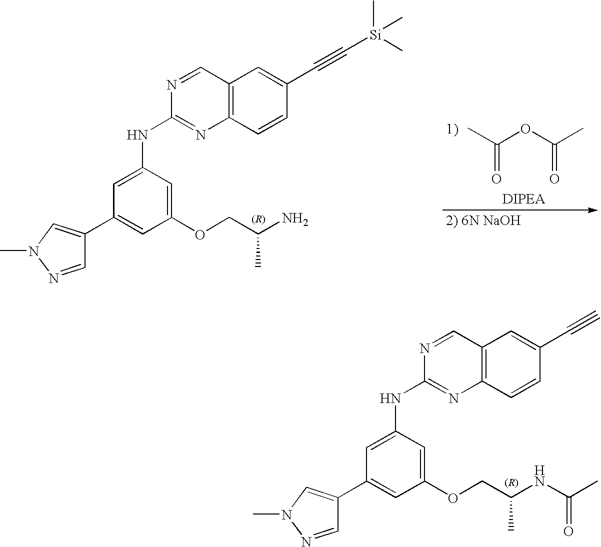

### Step 1: Preparation of (R)—N-(1-(3-(6-ethynylquinazolin-2-ylamino)-5-(1-methyl-1H-pyrazol-4-yl)phenoxy)propan-2-yl)acetamide

To the reaction mixture of (R)—N-(3-(2-aminopropoxy)-5-(1-methyl-1H-pyrazol-4-yl)phenyl)-6-((trimethylsilyl)ethynyl)quinazolin-2-amine (15 mg, 30 umole) in 0.5 ml of N,N-dimethylformamide, acetic anhydride (9 ul , 90 umole) and DIPEA (17 ul, 100 umole) were added. The reaction solution was stirred at room temperature for about 90 minutes or until done by LCMS. The crude product was deprotected with 60 ul of 6N NaOH solution for 5 minutes and then neutralized with 60 ul of acetic acid. The crude reaction mixture was filtered, purified on prep HPLC and lyophilized to give (R)—N-(1-(3-(6-ethynylquinazolin-2-ylamino)-5-(1-methyl-1H-pyrazol-4-yl)phenoxy)propan-2-yl)acetamide as TFA salt (4.2 mg). ES/MS m/z 441.2 (MH⁺).

### Example 619

### (Table 2): (R)—N-(1-(3-(6-ethynylquinazolin-2-ylamino)-5-(1-methyl-1H-pyrazol-4-yl)phenoxy)propan-2-yl)cyclopropanesulfonamide

The subject compound was prepared according to the general Scheme below

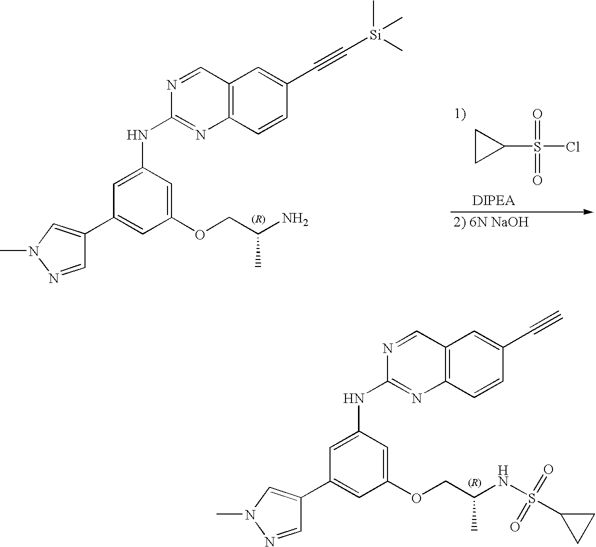

### Step 1: Preparation of (R)—N-(1-(3-(6-ethynylquinazolin-2-ylamino)-5-(1-methyl-1H -pyrazol-4-yl)-phenoxy)propan-2-yl)cyclopropanesulfonamide

To the reaction mixture of (R)—N-(3-(2-aminopropoxy)-5-(1-methyl-1H-pyrazol-4-yl)phenyl)-6-((trimethylsilyl)ethynyl)quinazolin-2-amine (15 mg, 30 umole) in 0.5 ml of N,N-dimethylformamide, cyclopropanesulfonyl chloride (10 ul , 98 umole) and DIPEA (35 ul, 200 umole) were added. The reaction solution was stirred at room temperature for 16 hours minutes or until done by LCMS. The crude product was deprotected with 60 ul of 6N NaOH solution for 5 minutes and then neutralized with 60 ul of acetic acid. The crude reaction mixture was filtered, purified on prep HPLC and lyophilized to give (R)—N-(1-(3-(6-ethynylquinazolin-2-ylamino)-5-(1-methyl-1H-pyrazol-4-yl)phenoxy)propan-2-yl)cyclopropanesulfonamideas TFA salt (4.3 mg). ES/MS m/z 503.2 (MH⁺).

### Example 724

### (Table 3): Preparation of N-methyl-4-(7-(piperidin-4-ylmethoxy)-6-(thiazol-2-yl)quinazolin-2-ylamino)benzamide

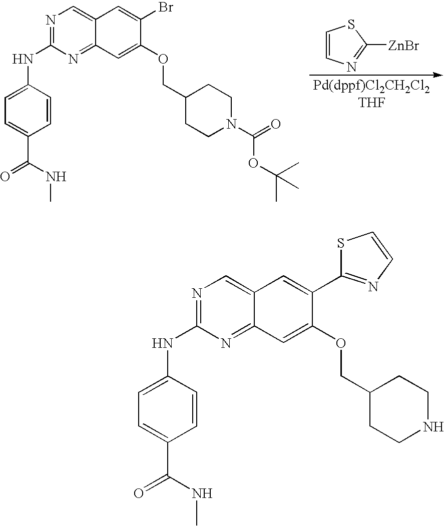

To a 0.05M solution of tert-butyl 4-((6-bromo-2-(4-(methylcarbamoyl)phenylamino) quinazolin-7-yloxy)methyl)piperidine-1-carboxylate (0.12 mmol) in THF was added 0.5M 2-thiazolylzincbromide (3.0 eq), Pd(dppf)Cl2CH2Cl2 (0.2 eq). The mixture was microwaved at 120° C. for 15 mins. Complete conversion to the product was seen by LC/MS. The mixture was concentrated and partitioned between ethyl acetate and water. The organic layer was dried (Na₂SO₄) and was concentrated. The residue was treated with 50% TFA/DCM and stirred for 10 min to remove tert-butylcarboxylate group. The crude compound was then purified by RP HPLC to give desired product N-methyl-4-(7-(piperidin-4-ylmethoxy)-6-(thiazol-2-yl)quinazolin-2-ylamino)benzamide. ES/MS m/z 475 (MH⁺).

### Example 729

### (Table 3): Preparation of N-methyl-4-(7-(piperidin-4-ylmethoxy)-6-(thiazol-2-yl)quinazolin-2-ylamino)benzamide

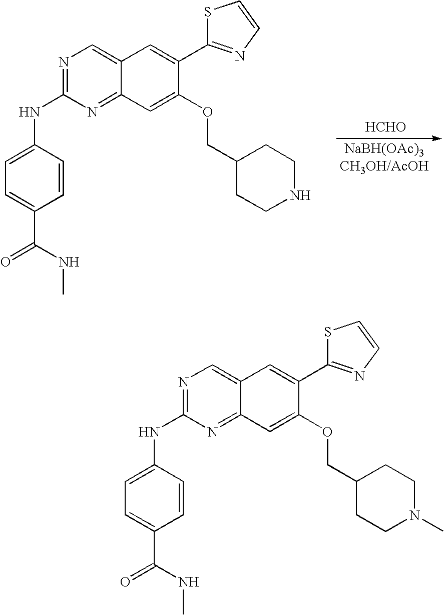

A solution of N-methyl-4-(7-(piperidin-4-ylmethoxy)-6-(thiazol-2-yl)quinazolin-2-ylamino)benzamide (0.05 mmol), formaldehyde (10 eq), catalytic amount acetic acid in methanol (2.0 ml) was stirred at ambient temperature for 1 h. Then sodium triacetoxyborohydride (2.0 eq) was added and reaction mixture was stirred at ambient temperature for 2 h. LCMS data showed reaction was complete. Solvent was removed under reduced pressure. The residue was taken into ethyl acetate and was washed with water, brine, dried and concentrated. Purification by RP HPLC gave desired product N-methyl -4-(7-(piperidin-4-ylmethoxy)-6-(thiazol-2-yl)quinazolin-2-ylamino)benzamide. ES/MS m/z 489 (MH⁺).

### Example 764

### (Table 3): Preparation of (S)—N-(3-((1H-1,2,4-triazol-1-yl)methyl)phenyl)-6-cyclopropyl-7-(pyrrolidin-3-yloxy)quinazolin-2-amine

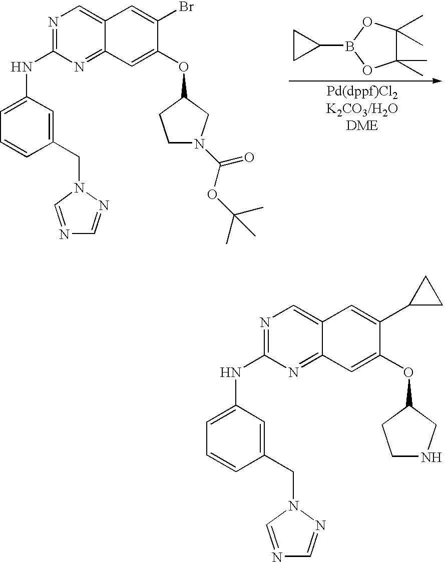

To a 0.06 M solution of (S)-tert-butyl 3-(2-(3-((1H-1,2,4-triazol-1-yl)methyl)phenylamino)-6-bromoquinazolin-7-yloxy)pyrrolidine-1-carboxylate (0.13 mmol) in DME was added 2-cyclopropyl-4,4,5,5-tetramethyl-1,3,2-dioxaborolane (10 eq), Pd(dppf)Cl2CH2Cl2 (0.2 eq), and 2.0M potassium carbonate aqueous solution (4.0 eq). The mixture was microwaved at 120° C. for 20 mins. Complete conversion to the product was confirmed by LC/MS. The mixture was partitioned between ethyl acetate and water. The organic layer was dried (Na₂SO₄) and was concentrated. The residue was treated with 50% TFA/DCM and stirred for 10 min to remove tert-butylcarboxylate group. The crude compound was then purified by RP HPLC to give desired product (S)—N-(3-((1H-1,2,4-triazol-1-yl)methyl)phenyl)-6-cyclopropyl-7-(pyrrolidin-3-yloxy)quinazolin-2-amine. ES/MS m/z 428 (MH⁺).

### Example 765

### (Table 3): Preparation of (S)—N-(3-((1H-1,2,4-triazol-1-yl)methyl)phenyl)-6-methyl-7-(pyrrolidin-3-yloxy)quinazolin-2-amine

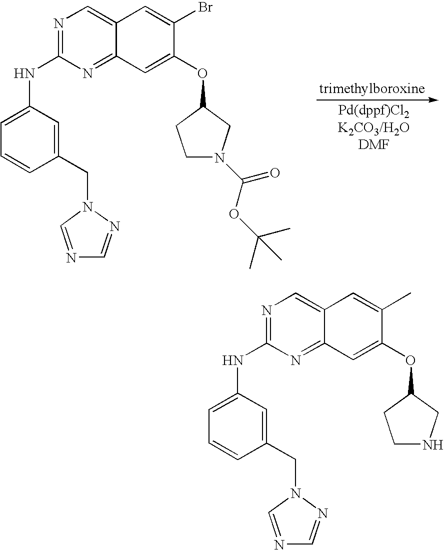

To a 0.06 M solution of (S)-tert-butyl 3-(2-(3-((1H-1,2,4-triazol-1-yl)methyl)phenylamino)-6-bromoquinazolin-7-yloxy)pyrrolidine-1-carboxylate (0.11 mmol) in DMF was added trimethylboroxine (4.0 eq), Pd(dppf)Cl2CH2Cl2 (0.1 eq), and 2.0M potassium carbonate aqueous solution (4.0 eq). The mixture was microwaved at 120° C. for 20 mins. Complete conversion to the product was seen by LC/MS. The mixture was partitioned between ethyl acetate and water. The organic layer was dried (Na₂SO₄) and was concentrated. The residue was treated with 50% TFA/DCM and stirred for 10 min to remove tert-butylcarboxylate group. The crude compound was then purified by R^{P} HPLC to give desired product (S)—N-(3-((1H-1,2,4-triazol-1-yl)methyl)phenyl)-6-methyl-7-(pyrrolidin-3-yloxy)quinazolin-2-amine. ES/MS m/z 402 (MH⁺).

### Example 767

### (Table 3): Preparation of (S)-2-(3-((1H-1,2,4-triazol-1-yl)methyl)phenylamino)-7-(pyrrolidin-3-yloxy)quinazoline-6-carbonitrile

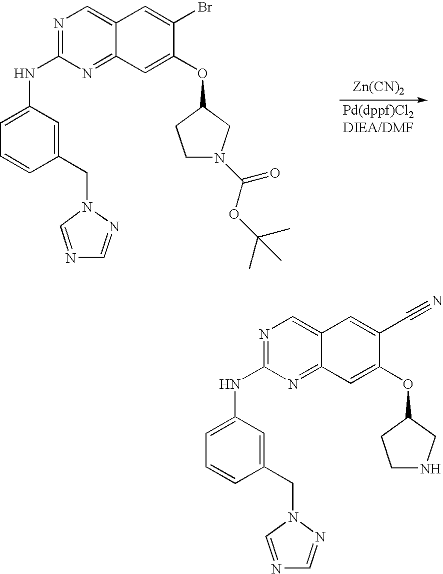

To a 0.1M solution of (S)-tert-butyl 3-(2-(3-((1H-1,2,4-triazol-1-yl)methyl)phenylamino)-6-bromoquinazolin-7-yloxy)pyrrolidine-1-carboxylate (0.18 mmol) in DMF was added zinc cyanide (5.0 eq), Pd(dppf)Cl2CH2Cl2 (0.2 eq), and diisopropylethyl amine (1.5 eq). The mixture was microwaved at 150° C. for 20 mins. Complete conversion to the product was seen by LC/MS. The mixture was partitioned between ethyl acetate and water. The organic layer was dried (Na₂SO₄) and was concentrated. The residue was treated with 50% TFA/DCM and stirred for 10 min to remove tert-butylcarboxylate group. The crude compound was then purified by RP HPLC to give desired product (S)-2-(3-((1H-1,2,4-triazol-1-yl)methyl)phenylamino)-7-(pyrrolidin-3-yloxy)quinazoline-6-carbonitrile. ES/MS m/z 413 (MH⁺).

### Example 797

### (Table 3): Preparation of 7-(2-chloropyridin-4-yloxy)-N-(3-fluorophenyl)-6-(thiazol-2-yl)quinazolin-2-amine

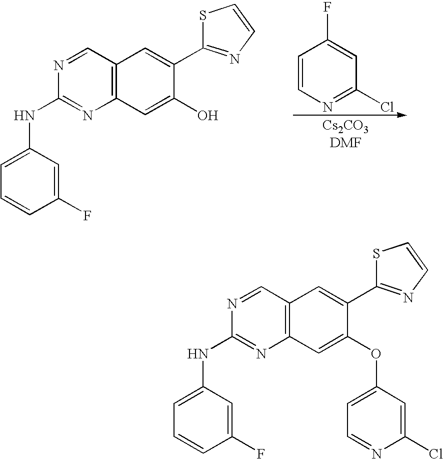

A 0.1 M suspension of 2-(3-fluorophenylamino)-6-(thiazol-2-yl)quinazolin-7-ol (0.15 mmol), 2-chloro-4-fluoropyridine (1.5 eq), Cesium carbonate (3.0 eq) in DMF was heated to 95° C. in an oil bath for 18 h. LCMS data indicated desired product formation. The mixture was partitioned between ethyl acetate and water. The organic layer was dried (Na₂SO₄) and was concentrated. The crude compound was then purified by RP HPLC to give desired product 7-(2-chloropyridin-4-yloxy)-N-(3-fluorophenyl)-6-(thiazol-2-yl)quinazolin-2-amine. ES/MS m/z 450 (MH⁺).

### Example 804

### (Table 3): Preparation of 6-ethynyl-N-(3-(morpholinomethyl)phenyl)-7-(pyridin-3-ylmethoxy)quinazolin-2-amine

The subject compound was prepared according to the general Scheme below:

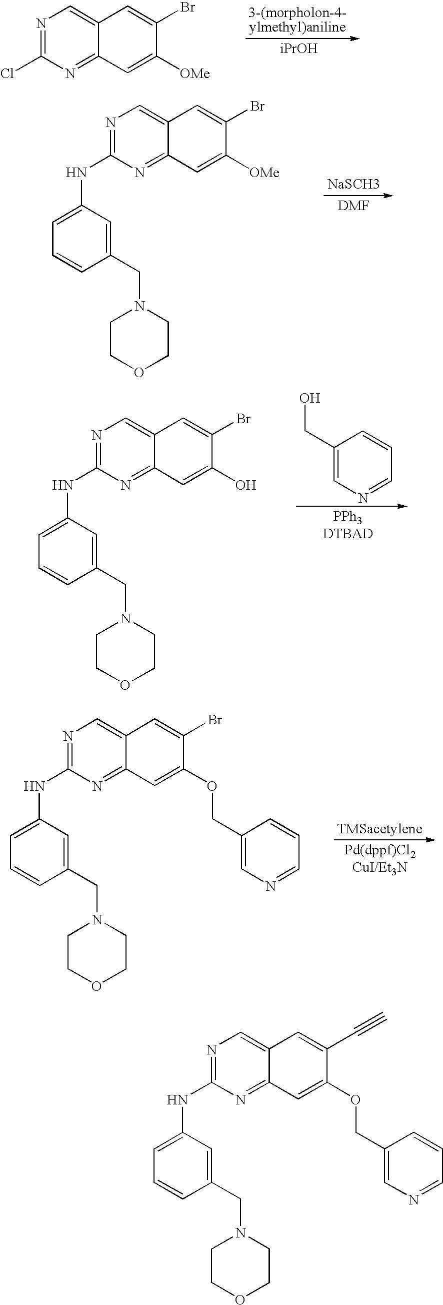

### A. Preparation of 6-bromo-7-methoxy-N-(3-(morpholinomethyl)phenyl)quinazolin-2-amine

To a 0.25M suspension of 6-bromo-2-chloro-7-methoxyquinazoline in isopropanol was added 3-(morpholin-4-ylmethyl)aniline (1.1 eq), and 4.0M hydrogen chloride in 1,4-dioxane (0.5 eq). The reaction mixture was heated to 95° C. in an oil bath for 15 h. Reaction mixture was diluted with ethyl acetate and filtered to collect desired product. ES/MS m/z 429/431 (MH⁺).

### B. Preparation of 6-bromo-2-(3-(morpholinomethyl)phenylamino)quinazolin-7-ol

A 0.2M suspension of 6-bromo-7-methoxy-N-(3-(morpholinomethyl)phenyl)quinazolin-2-amine (1.0 eq) and sodium thiomethoxide (4.0 eq) in DMF was heated to 90° C. in an oil bath for 10 h. The mixture was partitioned between ethyl acetate and water. The pH of aqueous phase was adjusted to 4 by adding saturated ammonium hydrochloride. Aqueous phase was extracted with ethyl acetate, and combined organic phase was washed with brine, dried over sodium sulfate, concentrated to give desired product. ES/MS m/z 415/417 (MH⁺).

### C. Preparation of 6-bromo-N-(3-(morpholinomethyl)phenyl)-7-(pyridin-3-ylmethoxy)quinazolin-2-amine

To a 0.55M solution of triphenylphosphine (3.0 eq) in THF was added di-tert-butylazodicarboxylate (3.0 eq). The mixture was stirred for 20 min at ambient temperature. 3-pyridylcarbinol (3.0 eq) was added, and reaction mixture was stirred at ambient temperature for another 30 min. Then, 6-bromo-2-(3-(morpholinomethyl)phenylamino) quinazolin-7-ol (1.0 eq) was added to reaction flask. The mixture was stirred at ambient temperature for additional 14 h. Solvent was removed under reduced pressure and the residue was triturated with ethyl acetate and filtered. Filter cake was rinsed with cold ethyl acetate and was air dried to give 6-bromo-N-(3-(morpholinomethyl)phenyl)-7-(pyridin-3-ylmethoxy)quinazolin-2-amine. ES/MS m/z 506/508 (MH⁺).

### D. Preparation of 6-ethynyl-N-(3-(morpholinomethyl)phenyl)-7-(pyridin-3-ylmethoxy)quinazolin-2-amine

To a 0.05M mixture of 6-bromo-N-(3-(morpholinomethyl)phenyl)-7-(pyridin-3-ylmethoxy)quinazolin-2-amine (0.1 mmol), triethylamine (0.4 ml), Pd(dppf)Cl2CH2Cl2 (0.1 eq), Copper(I) iodide (0.1 eq) in DMF was added trimethylsilylacetylene (10 eq). The mixture was microwaved at 120° C. for 15 min. Reaction mixture was diluted with ethyl acetate and was washed with water, brine, dried and concentrated. Without further purification, the crude compound was treated with tetramethylammonium fluoride (2.0 eq) in THF/isopropanol (10:1, 0.02M) at ambient temperature for 15 min. Solvent was removed under reduced pressure. The residue was taken into ethyl acetate and was washed with water, brine, dried and concentrated. The crude product was purified by R^{P} HPLC. Lyophilization gave desired product 6-ethynyl-N-(3-(morpholinomethyl)phenyl)-7-(pyridin-3-ylmethoxy)quinazolin-2-amine. ES/MS m/z 452 (MH⁺).

### Example 889

### (Table 3): Preparation of N-(3-fluorophenyl)-7-(1-methyl-1H-pyrazol-4-yl)-6-(thiazol-2-yl)quinazolin-2-amine

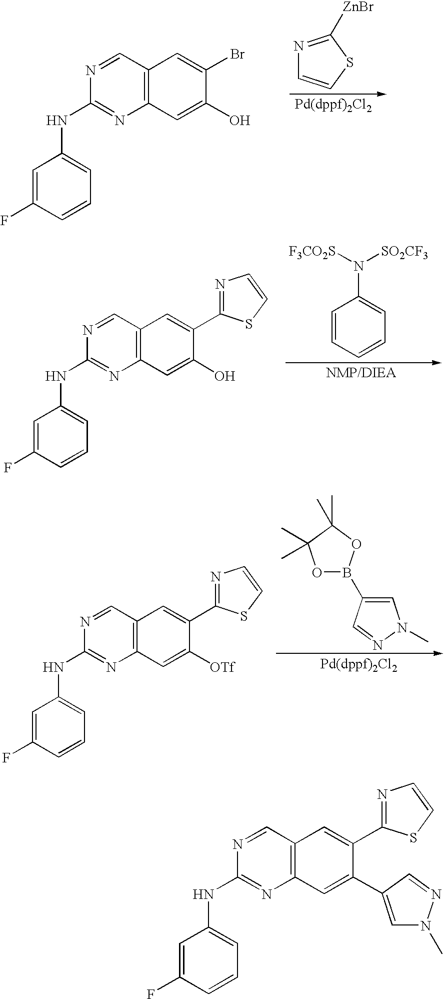

### A. Preparation of 2-(3-fluorophenylamino)-6-(thiazol-2-yl)quinazolin-7-ol

To 6-bromo-2-(3-fluorophenylamino)quinazolin-7-ol was added 0.5M 2-thiazolylzincbromide and the mixture was micro waved at 120° C. for 10 mins. Complete conversion to the product was seen by LC/MS. The mixture was concentrated and partitioned between ethyl acetate and water. The organic layer was dried (Na₂SO₄) and the product was isolated. ES/MS m/z 330 (MH⁺).

### B. Preparation of 2-(3-fluorophenylamino)-6-(thiazol-2-yl)quinazolin-7-yl trifluoromethanesulfonate

To a solution of 2-(3-fluorophenylamino)-6-(thiazol-2-yl)quinazolin-7-ol (1 eq) in NMP was added phenyltrifluoromethanesulfonate (1.2 eq) and DIEA (2.5 eq) and the reaction mixture was stirred over night at ambient temperature. The reaction mixture was then partitioned between ethyl acetate and water. The organic layers were washed with saturated sodium chloride and dried and concentrated. To the crude was added methylene chloride and few drops of methanol. The white solid hence formed was filtered to give 2-(3-fluorophenylamino)-6-(thiazol-2-yl)quinazolin-7-yl trifluoromethanesulfonate in 80% yield. ES/MS m/z 471 (MH⁺).

### C. Preparation of N-(3-fluorophenyl)-7-(1-methyl-1H-pyrazol-4-yl)-6-(thiazol-2-yl)quinazolin-2-amine

To a solution of 2-(3-fluorophenylamino)-6-(thiazol-2-yl)quinazolin-7-yl trifluoromethanesulfonate (1 eq) in DME was added 2M sodium carbonate solution and 4-(4,4,5,5,-tetramethyl-1,3,2-dioxaborolan-2-yl)-1methyl-pyrazole (3 eq) and Pd (dppf)₂Cl₂.CH₂Cl₂ (0.05 eq) and the mixture was micro waved for 10 min at 120° C. The reaction mixture was then partitioned between ethyl acetate and water. The organic layer was washed with brine, dried, concentrated and purified by semi-preparative HPLC to provide N-(3-fluorophenyl)-7-(1-methyl-1H-pyrazol-4-yl)-6-(thiazol-2-yl)quinazolin-2-amine ES/MS m/z 402 (MH⁺).

### Example 902

### (Table 3): N-(3-(6-(thiazol-2-yl)quinazolin-2-ylamino)phenyl)acetamide

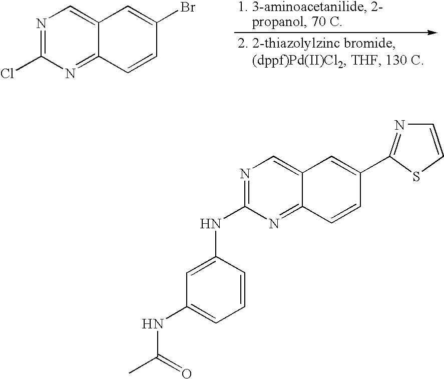

### Step 1. Displacement

A mixture of 6-bromo-2-chloroquinazoline (1 eq) and 3-aminoacetanilide (0.9 eq) in 2-propanol was heated to 70° C. for 16 hr and concentrated to give the crude product which was used without further purification.

### Step 2. Negishi

To the product of step 1 was added a 0.5 M THF solution of 2-thiazolylzinc bromide (4.0 eq) and [1,1′-bis(diphenylphosphino)ferrocene]dichloropalladium(II) complex with DCM (0.10 eq). The reaction was microwaved at 130° C. for 10 min. The mixture was diluted with ethyl acetate and washed with aqueous EDTA pH-9 buffer. The organic phase was dried over sodium sulfate, and concentrated. Purification by reverse-phase HPLC and lyophilization gave the desired product as its TFA salt. ES/MS m/z 392 (MH⁺).

### 6-Bromo-2,7-dichloroquinazoline

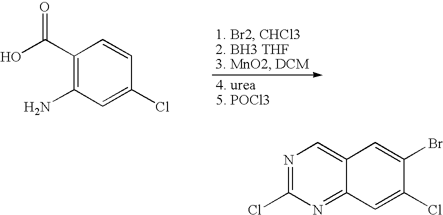

Step 1: Bromination

To a suspension of 2-amino-4-chlorobenzoic acid (2 g, 11.6 mmol) in chloroform (120 mL) was added dropwise bromine (1.1 equiv.) in chloroform (12 mL) solution. The mixture was stirred at RT for 16 hrs. The resulting white solid was collected by filtration and washed thoroughly with DCM until the filtrate was colorless. The solid was air-dried to give 3.35 g of white powder as HBr salt of 2-amino-5-bromo-4-chlorobenzoic acid (87% yield). ES/MS m/z 250/252 (MH⁺).

Step 2: Reduction

To the above intermediate (3.35 g, 10.1 mmol) in THF (40 mL) at 0° C. was added borane-THF complex solution (1 M in THF, 40 mL, 4 equiv.). The mixture was stirred at RT for 18 hrs. Excess reagent was quenched by addition of ethanol (20 mL) slowly. Water was added and the pH (˜3) was adjusted by adding sodium bicarbonate (sat. aq.) to pH 7. Volatiles were removed under reduced pressure. The resulting mixture was extracted with DCM. The organic extracts were combined, washed with brine, dried with sodium sulfate and concentrated to give the crude product as a white solid. ES/MS m/z 236/238 (MH⁺)

Step 3: Oxidation

To the above intermediate (10.1 mmol) in DCM (80 mL) was added manganese (IV) oxide (MnO₂, 6 g, 70 mmol). The mixture was stirred at RT under argon for 40 hrs. The mixture was filtered through diatomaceous earth and washed thoroughly with DCM. The filtrate was concentrated in vacuo to give crude 2-amino-5-bromo-4-chlorobenzyl alcohol (3.3 g, orange solid) which was used for the next step without further purification. ES/MS m/z 234/236 (MH⁺).

Step 4: Cyclization

A mixture of crude 2-amino-5-bromo-4-chlorobenzyl alcohol (3.3 g, obtained from Step 3) and urea (10.5 g, 15 equiv.) was heated to 170° C. with vigorous stirring for 1 hr. The reaction was cooled to RT and water was added. The solid was collected by filtration. The filtered solid was air-dried to give the crude product as a yellow powder (2.18 g, crude). ES/MS m/z 259/261 (MH⁺).

Step 5: Chlorination

To the above crude material was added phosphorus oxychloride (POCl₃, 25 mL) and heated to 110° C. for 30 min. The resulting mixture was cooled to RT and concentrated in vacuo to nearly dryness. Ice water was added and pH was adjusted to ˜8 using sodium bicarbonate. The mixture was extracted with DCM and the extract was dried with sodium sulfate and concentrated in vacuo. Crude 6-bromo-2,7-dichloro-quinazoline was purified by flash chromatography over silica gel eluting with 2:1 hexanes:ethyl acetate. ES/MS m/z 279 (MH⁺).

### Example 904

### (Table 3): 3-(7-chloro-6-ethynylquinazolin-2-ylamino)-N-methyl-5-(1-methyl-1H-pyrazol-4-yl)benzamide

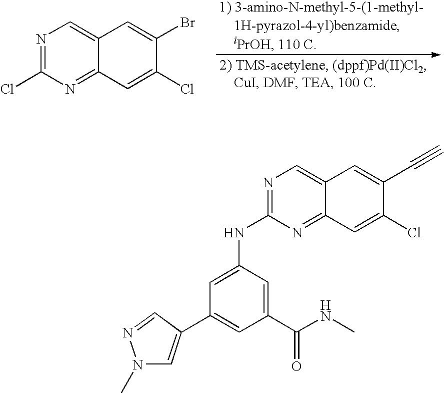

### Step 1. Displacement

A mixture of 6-bromo-2,7-dichloroquinazoline (1 eq) and 3-amino-N-methyl-5-(1-methyl-1H-pyrazol-4-yl)benzamide (1 eq) in 2-propanol was heated to 110° C. for 16 hr and concentrated to give the crude product which was used without further purification.

### Step 2. Sonogashira and Desilylation

To a 0.15 M solution of the product of step 1 in de-gassed 1:1 DMF:TEA was added TMS-acetylene (4.0 eq); copper(I) iodide (0.10 eq); and [1,1′-bis(diphenylphosphino)ferrocene]dichloropalladium(II) complex with DCM (0.050 eq). The reaction was stirred at 100° C. for 40 min. The mixture was concentrated and re-dissolved in 3:2 tetrahydrofuran:methanol to make a 0.15 M solution. A 6M aqueous solution of sodium hydroxide (2.5 eq) was added, and the mixture was stirred for 20 min. Volatiles were removed under reduced pressure. The residue was purified by reverse-phase HPLC and lyophilized to give the desired product as its TFA salt. ES/MS m/z 417.1 (MH⁺).

### Example 905

### (Table 3): 3-(6-ethynyl-7-(trifluoromethyl)quinazolin-2-ylamino)-N-methyl-5-(1-methyl-1H-pyrazol-4-yl)benzamide

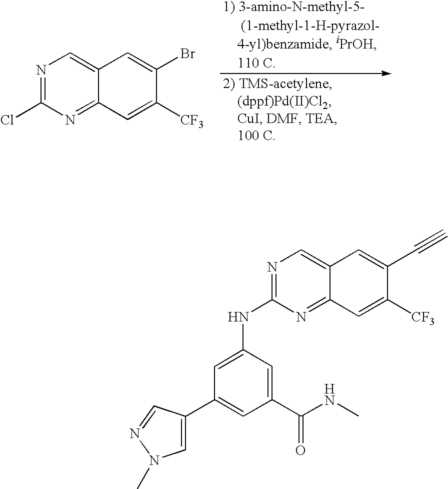

3-(6-Ethynyl-7-(trifluoromethyl)quinazolin-2-ylamino)-N-methyl-5-(1-methyl-1H-pyrazol-4-yl)benzamide was prepared following Examples 62 and 63. ES/MS m/z 451.0 (MH⁺).

### Example 907

### (Table 3): 2-(3-fluorophenylamino)-6-(thiazol-2-yl)quinazoline-7-carbaldehyde

The subject compound was prepared according to the general Scheme below:

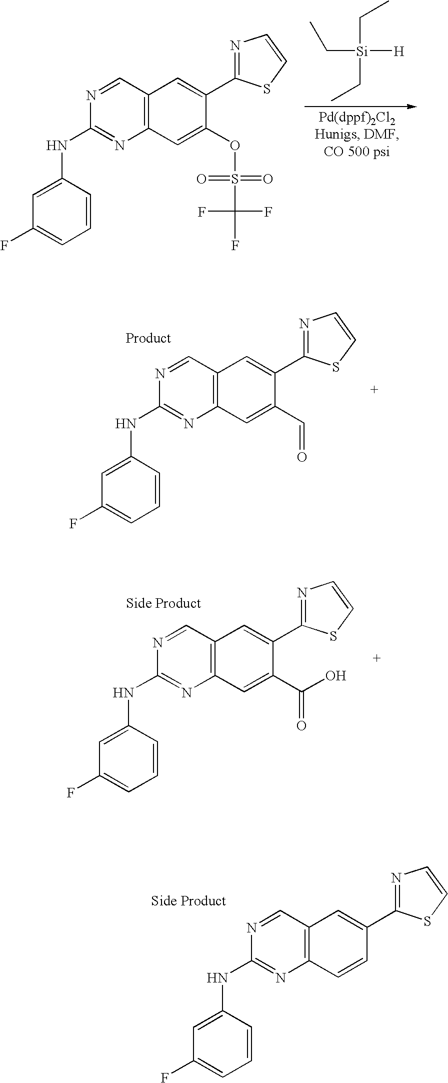

### 2-(3-fluorophenylamino)-6-(thiazol-2-yl)quinazoline-7-carbaldehyde

To the reaction mixture of 2-(3-fluorophenylamino)-6-(thiazol-2-yl)quinazolin-7-yl trifluoromethanesulfonate (50 mg, 0.106 mmol) in 1.4 ml of DMF in a steel bomb was added Pd(dppf)₂Cl₂ (8.7 mg, 0.0106 mmol), triethylsilane (37 mg , 0.318 mmol) and TEA (0.037 ml, 0.265 mmol). The steel bomb was sealed and carefully CO was added to 500 psi. and slowly released (3× in a Hood) then recharged to 500 psi. and stirred at 50-55° C. for 18 hours. This reaction mixture was cooled and vented in a hood. The crude reaction mixture was filtered, purified on prep HPLC and lyophilized to give, 2-(3-fluorophenylamino)-6-(thiazol-2-yl)quinazoline-7-carbaldehyde as TFA salt (6.0 mg). ES/MS m/z 351 (MH⁺).

### Side Products

In addition two side products were also collect on prep HPLC and lyophilized to give, 2-(3-fluorophenylamino)-6-(thiazol-2-yl)quinazoline-7-carboxylic acid as TFA salt (8.0 mg) ES/MS m/z 367 (MH⁺) and N-(3-fluorophenyl)-6-(thiazol-2-yl)quinazolin-2-amine as TFA salt (3.3 mg) ES/MS m/z 323 (MH⁺).

### Example 908

### (Table 3): N-(3-fluorophenyl)-7-(piperazin-1-ylmethyl)-6-(thiazol-2-yl)quinazolin-2-amine

The subject compound was prepared according to the general Scheme below:

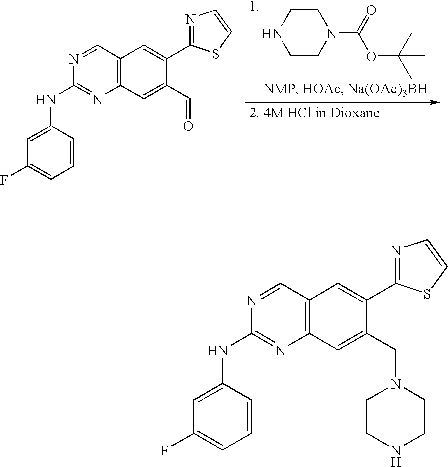

### Step 1: tert-butyl 4-((2-(3-fluorophenylamino)-6-(thiazol-2-yl)quinazolin-7-yl)methyl)piperazine-1-carboxylate

To the reaction mixture of 2-(3-fluorophenylamino)-6-(thiazol-2-yl)quinazoline-7-carbaldehyde (12 mg, 0.034 mmol) in 0.75 ml of NMP add acetic acid (0.070 ml , 1.02 mmol) and tert-butyl piperazine-1-carboxylate (65 mg, 0.35 mmol). This reaction mixture was stirred at room temperature for about 16 hours. To this reaction solution was added sodium triacetoxy borohydride (18 mg, 0.085 mmol) and stirred at room temperature 2 hours or until done by LCMS. The crude reaction mixture with product, tert-butyl 4-((2-(3-fluorophenylamino)-6-(thiazol-2-yl)quinazolin-7-yl)methyl)piperazine-1-carboxylate was use in the next step without purification. ES/MS m/z 521 (MH⁺).

### Step 2: N-(3-fluorophenyl)-7-(piperazin-1-ylmethyl)-6-(thiazol-2-yl)quinazolin-2-amine

To the crude reaction mixture of tert-butyl 4-((2-(3-fluorophenylamino)-6-(thiazol-2-yl)quinazolin-7-yl)methyl)piperazine-1-carboxylate (0.034 mmol) was added 4M HCl in Dioxane (4.0 ml, 16.0 mmol). The reaction mixture was stirred at room temperature for 1 hours or until done by LCMS. The crude reaction mixture was concentrate, about 1 ml of DMF was added, filtered, purified on prep HPLC and lyophilized to give, N-(3-fluorophenyl)-7-(piperazin-1-ylmethyl)-6-(thiazol-2-yl)quinazolin-2-amine as TFA salt (1.7 mg). ES/MS m/z 421 (MH⁺).

### Example 909

### (Table 3) (2-(3-fluorophenylamino)-6-(thiazol-2-yl)quinazolin-7-yl)(piperazin-1-yl)methanone

The subject compound was prepared according to the general Scheme below:

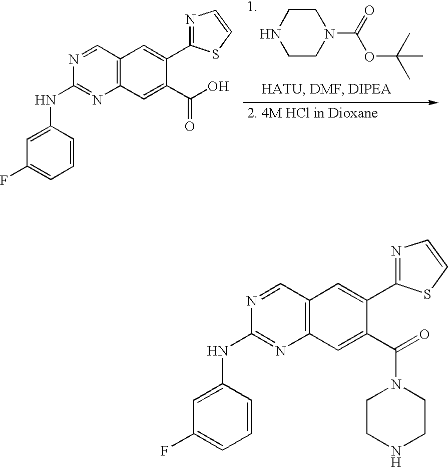

### Step 1: tert-butyl 4-(2-(3-fluorophenylamino)-6-(thiazol-2-ylquinazoline-7-carbonyl)piperazine-1-carboxylate

To the reaction mixture of 2-(3-fluorophenylamino)-6-(thiazol-2-yl)quinazoline-7-carboxylic acid (7 mg, 0.019 mmol) in 0.4 ml of DMF add HATU (18.2 mg, 0.048 mmol), DIPEA (0.014 ml, 0.076 mmol) and stir at room temperature for about 3-5 minutes. To the above reaction mixture add tert-butyl piperazine-1-carboxylate (14 mg, 0.076 mmol) and stir at room temperature for 2 hours or until done by LCMS. The crude reaction mixture with product, tert-butyl 4-(2-(3-fluorophenylamino)-6-(thiazol-2-yl)quinazoline-7-carbonyl)piperazine-1-carboxylate was use in the next step without purification. ES/MS m/z 535 (MH⁺).

### Step 2: (2-(3-fluorophenylamino)-6-(thiazol-2-yl)quinazolin-7-yl)(piperazin-1-yl)methanone

To the crude reaction mixture of tert-butyl 4-(2-(3-fluorophenylamino)-6-(thiazol-2-yl)quinazoline-7-carbonyl)piperazine-1-carboxylate (0.019 mmol) was added 4M HCl in Dioxane (2.0 ml, 8.0 mmol). The reaction mixture was stirred at room temperature for 1 hours or until done by LCMS. The crude reaction mixture was concentrate, about 1 ml of DMF was added, filtered, purified on prep HPLC and lyophilized to give, (2-(3-fluorophenylamino)-6-(thiazol-2-yl)quinazolin-7-yl)(piperazin-1-yl)methanone as TFA salt (2.1 mg). ES/MS m/z 435 (MH⁺).

### Example 913

### (Table 3): N-(3-fluorophenyl)-7-(piperidin-4-yloxy)-6-(pyridin-2-yl)quinazolin-2-amine

The subject compound was prepared according to the general Scheme below:

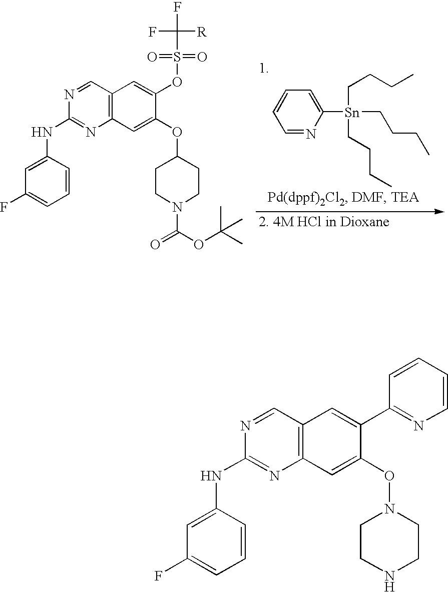

### Step 1: tert-butyl 4-(2-(3-fluorophenylamino)-6-(pyridin-2-yl)quinazolin-7-yloxy)piperidine-1-carboxylate

To the reaction mixture of tert-butyl 4-(2-(3-fluorophenylamino)-6-(trifluoromethylsulfonyloxy)quinazolin-7-yloxy)piperidine-1-carboxylate (30 mg, 0.058 mmol) in 0.5 ml of DMF was added Pd(dppf)₂Cl₂ (9.5 mg, 0.0116 mmol), 2-(tributylstannyl)pyridine (66 mg, 0.174 mmol) and last add TEA (0.021 ml, 0.145 mmol). This reaction mixture was stirred at 105° C. for 1 hours or microwaved at 120° C. for 800 seconds until done by LCMS. The crude reaction mixture with product, tert-butyl 4-(2-(3-fluorophenylamino)-6-(pyridin-2-yl)quinazolin-7-yloxy)piperidine-1-carboxylate was use in the next step without purification. ES/MS m/z 516 (MH⁺).

### Step 2: N-(3-fluorophenyl)-7-(piperidin-4-yloxy)-6-(pyridin-2-yl)quinazolin-2-amine

To the crude reaction mixture of, tert-butyl 4-(2-(3-fluorophenylamino)-6-(pyridin-3-yl)quinazolin-7-yloxy)piperidine-1-carboxylate (0.058 mmol) was added 4M HCl in Dioxane (3.0 ml, 12.0 mmol). The reaction mixture was stirred at room temperature for 1 hours or until done by LCMS. The crude reaction mixture was concentrate, about 1 ml of DMF was added, filtered, purified on prep HPLC and lyophilized to give, N-(3-fluorophenyl)-7-(piperidin-4-yloxy)-6-(pyridin-2-yl)quinazolin-2-amine as TFA salt (4.6 mg). ES/MS m/z 416 (MH⁺).

### Example 916

### (Table 3): N-(3-fluorophenyl)-7-(piperidin-4-yloxy)-6-(pyridin-3-yl)quinazolin-2-amine

The subject compound was prepared according to the general Scheme below:

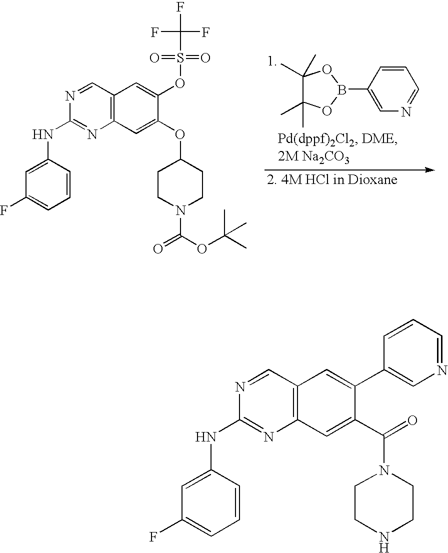

### Step 1: tert-butyl 4-(2-(3-fluorophenylamino)-6-pyridin-3-yl)quinazolin-7-yloxy)piperidine-1-carboxylate

To the reaction mixture of, tert-butyl 4-(2-(3-fluorophenylamino)-6-(trifluoromethylsulfonyloxy)quinazolin-7-yloxy)piperidine-1-carboxylate (30 mg, 0.058 mmol) in 0.7 ml of DME was added Pd(dppf)₂Cl₂ (9.5 mg, 0.0116 mmol), 3-(4,4,5,5-tetramethyl-1,3,2-dioxaborolan-2-yl)pyridine (36 mg, 0.174 mmol) and last add 2M Na₂CO₃ (0.2 ml, 0.4 mmol). This reaction mixture was stirred at 100° C. for 3 hours or until done by LCMS. To the crude reaction mixture add 1.5 ml of methanol, transfer the reaction and concentrate to solid to give a crude product, tert-butyl 4-(2-(3-fluorophenylamino)-6-(pyridin-3-yl)quinazolin-7-yloxy)piperidine-1-carboxylate used in next step without purification. ES/MS m/z 516 (MH⁺).

### Step 2: N-(3-fluorophenyl)-7-(piperidin-4-yloxy)-6-(pyridin-3-yl)quinazolin-2-amine

To the crude reaction mixture of tert-butyl 4-(2-(3-fluorophenylamino)-6-(pyridin-3-yl)quinazolin-7-yloxy)piperidine-1-carboxylate (0.058 mmol) was added 4M HCl in Dioxane (4.0 ml, 16.0 mmol). The reaction mixture was stirred at room temperature for 1 hours or until done by LCMS. The crude reaction mixture was concentrate, about 1 ml of DMF was added, filtered, purified on prep HPLC and lyophilized to give N-(3-fluorophenyl)-7-(piperidin-4-yloxy)-6-(pyridin-3-yl)quinazolin-2-amine as TFA salt (8.3 ing). ES/MS m/z 416 (MH⁺).

### Example 920

### (Table 3): (R)-1-(2-(3-fluorophenylamino)-7-(piperidin-4-yloxy)quinazolin-6-yl)pyrrolidin-3-ol

The subject compound was prepared according to the general Scheme below:

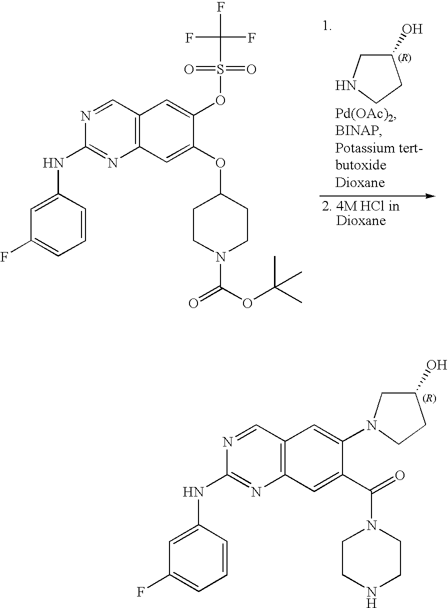

### Step 1: (R)-tert-butyl 4-(2-(3-fluorophenylamino)-6-(3-hydroxypyrrolidin-1-yl)quinazolin-7-yloxy)piperidine-1-carboxylate

To the reaction mixture of Pd(OAc)₂ (9.5 mg, 0.0116 mmol) and BINAP (10.8 mg, 0.0174 mmol) add 0.5 ml of dioxane and stir for 3-5 minutes at room temperature. To this reaction mixture add, tert-butyl 4-(2-(3-fluorophenylamino)-6-(trifluoromethylsulfonyloxy)quinazolin-7-yloxy)piperidine-1-carboxylate (30 mg, 0.058 mmol), (R)-pyrrolidin-3-ol (20 mg, 0.232 mmol) and then last add potassium tertbutoxide (19.5 mg, 0.174 mmol). This reaction mixture was stirred at 90° C. for 5 hours or until done by LCMS. The crude reaction mixture with product, (R)-tert-butyl 4-(2-(3-fluorophenylamino)-6-(3-hydroxypyrrolidin-1-yl)quinazolin-7-yloxy)piperidine-1-carboxylate was use in the next step without purification. ES/MS m/z 524 (MH⁺).

### Step 2: (R)-1-(2-(3-fluorophenylamino)-7-(piperidin-4-yloxy)quinazolin-6-yl)pyrrolidin-3-ol

To the crude reaction mixture of (R)-tert-butyl 4-(2-(3-fluorophenylamino)-6-(3-hydroxypyrrolidin-1-yl)quinazolin-7-yloxy)piperidine-1-carboxylate (0.058 mmol) was added 4M HCl in Dioxane (3.0 ml, 12.0 mmol). The reaction mixture was stirred at room temperature for 1 hours or until done by LCMS. The crude reaction mixture was concentrate, about 1 ml of DMF was added (if needed add about 0.2 ml of water), filter, purified on prep HPLC and lyophilized to give, (R)-1-(2-(3-fluorophenylamino)-7-(piperidin-4-yloxy)quinazolin-6-yl)pyrrolidin-3-ol as TFA salt (1.1 mg). ES/MS m/z 424 (MH⁺).

### Example 997

### (Table 3): Preparation of N-(3-fluorophenyl)-7-(piperidin-4-yloxy)-6-(thiazol-2-yl)quinazolin-2-amine

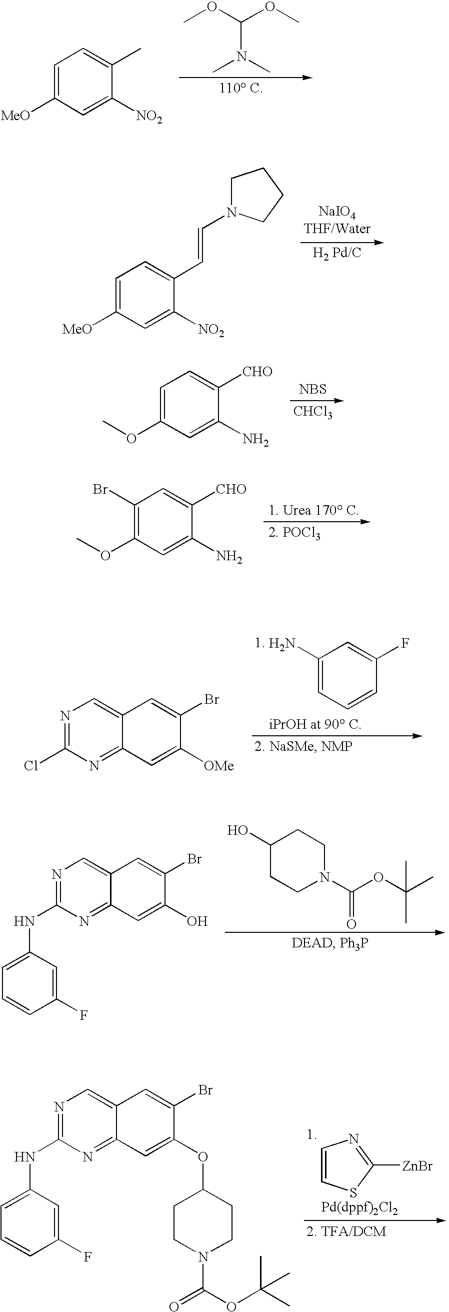

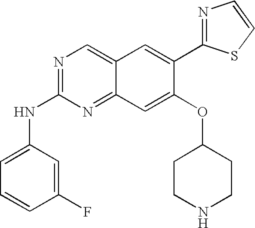

### A. Preparation of 4-methoxy-2-aminobenzaldehyde

To a mixture of 4-methyl-3-nitroanisole in pyrrolidine (1.9 eq) was added N,Ndimethylformamidedimethylacetal and the mixture was heated to 120° C. or 16 h. The formation of the enaminone was confirmed by TLC. The mixture was cooled to RT and poured in to ice and was then extracted with ethyl acetate, dried (Na₂SO₄) and concentrated. To the crude enaminone in THF was added NaIO₄ (2.5 eq) dissolved in water while maintaining the reaction temperature at 40° C. with an ice/water bath. After the addition the pale yellow suspension was stirred at 35° C. for 16 h. The reaction mixture was then concentrated and the aqueous phase was then extracted with ethyl acetate , dried (Na₂SO₄) and the crude 4-methoxy-2-nitrobenzaldehyde was obtained as a brown solid. The crude brown solid was purified on silica gel to get the product. The hence obtained 4-methoxy-2-nitrobenzaldehyde was hydrogenated in methanol with catalytic amounts of 10% Pd/C to give the 4-methoxy-2-aminobenzaldehyde in quantitative yield. ES/MS m/z 152 (MH⁺).

### B. Preparation of 5-bromo-4-methoxy-2-nitrobenzaldehyde

To 4-methoxy-2-aminobenzaldehyde in chloroform was added NBS(1 eq) and the reaction goes to completion by LC/MS and ¹H NMR. To the reaction mixture was added dichloromethane and water and the separated organic layer was dried (Na₂SO₄) and concentrated to give the 5-bromo-4-methoxy-2-nitrobenzaldehyde. ES/MS m/z 229/231 (MH^{÷}).

### C. Preparation of 2-chloro-6-bromo-7-methoxyquinazoline

5-bromo-4-methoxy-2-aminobenzaldehyde (1.0 eq) and urea (8.0 eq) were stirred at 170° C. for 1 h. The resulting solid was returned to ambient temperature, stirred in water for 20 min, and filtered. This was repeated for a total of three washes. The solid was dried in a desiccator to give the 6-bromo-7-methoxyquinazolin-2-ol as the desired product. A 0.50M solution of 6-bromo-7-methoxyquinazolin-2-ol in phosphorus oxychloride was stirred at 110° C. for 1.5 h. Volatiles were removed under reduced pressure. Ice water was added and the precipitate was filtered off, rinsed with water, and dried under high vacuum to yield the 2-chloro-6-bromo-7-methoxyquinazoline as a yellow solid. ES/MS m/z 272/274 (MH⁺).

### D. Preparation of 6-bromo-2-(3-fluorophenylamino)quinazolin-7-ol

To a solution of 2-chloro-6-bromo-7-methoxyquinazoline in isopropanol was added 3-fluoroaniline (1.0 eq). The reaction was stirred at 90° C. for 18 hours. The hydrochloride was collected by vacuum filtration and air dried to give a crude material which can be used for further chemical modifications. The pure material was obtained by HPLC purification. To the crude product was added NMP and NaSMe(4 eq) and the mixture was heated to 80° C. for 2 h. The conversion in to the phenol was observed by LC/MS. The reaction mixture was cooled and to it was added ethyl acetate and 1N HCl to bring it to neutral pH. The 6-bromo-2-(3-fluorophenylamino)quinazolin-7-ol crashed out as a solid which was filtered and dried on high vacuum to give the product in quantitative yield. ES/MS m/z 333/335 (MH⁺).

### E. Preparation of tert-butyl 4-(6-bromo-2-(3-fluorophenylamino)quinazolin-7-yloxy)piperidine-1-carboxylate

To a mixture of DEAD (2 eq) and triphenylphosphine (2 eq) was added tert-butyl 4-hydroxypiperidine-1-carboxylate (4 eq) and to the resulting mixture was added 6-bromo-2-(3-fluorophenylamino)quinazolin-7-ol and was stirred for 16 h at RT. The formation of the product was observed on LC/MS. The mixture was then concentrated and purified on silica gel to obtain tert-butyl 4-(6-bromo-2-(3-fluorophenylamino)quinazolin-7-yloxy)piperidine-1-carboxylate as the product. ES/MS m/z 517/519 (MH⁺).

### F. Preparation of N-(3-fluorophenyl)-7-(piperidin-4-yloxy)-6-(thiazol-2-yl)quinazolin-2-amine

To tert-butyl 4-(6-bromo-2-(3-fluorophenylamino)quinazolin-7-yloxy)piperidine-1-carboxylate (50 mgs) was added 0.5M 2-thiazolylzincbromide and the mixture was micro waved at 120° C. for 10 mins. Complete conversion to the product was seen by LC/MS. The mixture was concentrated and partitioned between ethyl acetate and water. The organic layer was dried (Na₂SO₄) and the product was isolated. To the crude product was added 30% TFA/DCM and stirred for 2 h for the deprotection of the tert-butyl group. The formation of N-(3-fluorophenyl)-7-(piperidin-4-yloxy)-6-(thiazol-2-yl)quinazolin-2-amine was observed by LC/MS. It was then purified by preparative chromatography to give the product. ES/MS m/z 421 (MH⁺).

### INTERMEDIATE for 5-Chloro Compounds: 5-chloro-2-(methylsulfonyl)-6-((trimethylsilyl)ethynyl)quinazoline

The subject compound was prepared according to the general Scheme below

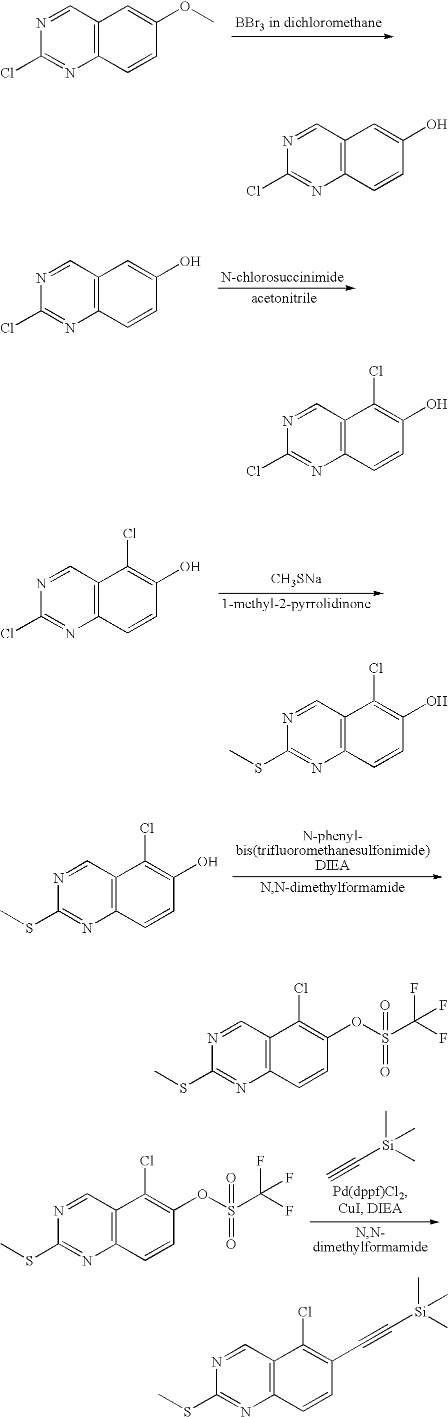

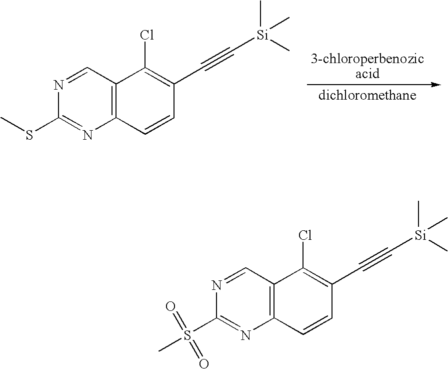

### Step 1: Preparation of 2-chloroquinazolin-6-ol

To the solution of 2-chloro-6-methoxy quinazoline (1 eq) in dichloromethane was added the solution of 1 M of boron tribromide (2 eq) in dichloromethane slowly over 3 minutes at 0° c. The reaction mixture was stirred at 0° c. for 20 minutes and heated at 40° c. for 16 hours. The deprotection of the methyl ether went to completion and was monitored by LC/MS. Removal of all solvent in vaccuo, followed by quenching with water and diluted NaHCO₃ solution at 0° c., and extraction of aqueous phase with ethyl acetate and drying of combined organic extracts over Na₂SO₄ and subsequent removal of ethyl acetate in vaccuo yielded the desired product that was dried under vacuum to give 2-chloroquinazolin-6-ol as brown solid. ES/MS m/z 181.0 (MH⁺).

### Step 2: Preparation of 2,5-dichloroquinazolin-6-ol

To the solution of 2-chloroquinazolin-6-ol (3.61 g, 20 mmole) in acetonitrile was added N-chlorosuccinimide (2.67 g, 20 mmole) and the mixture was stirred at room temperature and became a deep brown reaction solution. The reaction went to completion in 30 minutes to give 2,5-dichloroquinazolin-6-ol (85% of the desired isomer) that was observed by LC/MS and the structure was confirmed by ¹HNMR. The reaction solution was concentrated and then dissolved into 300 ml of ethyl acetate. The organic phase was washed with diluted hydrochloric acid (2×70 ml), water (2×50 ml) and brine (50 ml), then dried over Na₂SO₄ and evaporated in vacuo to give a brown solid (3.1 g) that was purified by column chromatography over silica gel with ethyl acetate:hexane (30:70) to give yellow powder as 2,5-dichloroquinazolin-6-ol (1.92 g). ES/MS m/z 215.0 (MH⁺).

### Step 3: Preparation of 5-chloro-2-(methylthio) quinazolin-6-ol

To the solution of 2,5-dichloroquinazolin-6-ol (1.75 g, 7.41 mmole) in 23 ml of N-methyl-2-pyrrolidone, sodium thiomethoxide (1.5 g, 22.23 mmole) was added. The reaction mixture was stirred at 60° c. for overnight. The reaction mixture was poured into 100 ml of saturated NaHCO₃ solution and extracted with ethyl acetate (2×150 ml). The combined organic layers were washed with diluted NaHCO₃ solution (80 ml), water (2×30 ml) and brine (40 ml), then dried over Na₂SO₄ and evaporated in vacuo to give a brown glue that was recrystallized in hexane/dichloromethane to give 5-chloro-2-(methylthio) quinazolin-6-ol as brown solid. ES/MS m/z 227.0 (MH⁺).

### Step 4: Preparation of 5-chloro-2-(methylthio) quinazolin-6-yl trifluoromethanesulfonate

To the solution of 5-chloro-2-(methylthio) quinazolin-6-ol (1.98 g, 7.93 mmole) in 30 ml of N,N-dimethylformamide at 0° c. was added N-phenyl-bis (trifluoromethanesulfonimide) (2.55 g, 7.13 mmole) and DIPEA (2.76 ml, 15.86 mmole). The resulting solution was stirred at room temperature for 2.5 hour and became a deep brown reaction solution. The reaction solution was partitioned between 250 ml of ethyl acetate and 80 ml of diluted hydrochloric acid. The organic layer was washed with diluted hydrochloric acid (60 ml), water (50 ml) and brine (50 ml), then dried over Na₂SO₄ and evaporated in vacuo to give a brown solid (4.72 g) that was purified by column chromatography over silica gel with ethyl acetate:hexane (10:90) to give beige color solid as 5-chloro-2-(methylthio) quinazolin-6-yl trifluoromethanesulfonate (2.8 g). ES/MS m/z 358.8 (MH⁴).

### Step 5: Preparation of 5-chloro-2-(methylthio)-6-((trimethylsilyl)ethynyl) quinazoline

To the reaction mixture of 5-chloro-2-(methylthio) quinazolin-6-yl trifluoromethanesulfonate (2.05 g, 5.39 mmole), Pd(dppf)Cl₂ (440 mg, 0.539 mmole), CuI (220 mg, 1.15 mmole) and DIPEA (3.6 ml, 20.7 mmole) in 36 ml of N,N-dimethylformamide was added trimethylsilylacetylene (2.2 ml, 16.2 mmole). The reaction mixture was stirred at 100° c. for 1.5 hour. The crude reaction mixture was partitioned between 300 ml of ethyl acetate and 80 ml of diluted hydrochloric acid. The organic layer was washed with diluted hydrochloric acid (60 ml), water (50 ml) and brine (50 ml), then dried over Na₂SO₄ and evaporated in vacuo to give a brown glue (−2.5 g) that was purified by column chromatography over silica gel with ethyl acetate:hexane (10:90) to give 5-chloro-2-(methylthio)-6-((trimethylsilyl)ethynyl)quinazoline (723 mg) as yellow solid . ES/MS m/z 351.0 (MH⁺).

### Step 6: Preparation of 5-chloro-2-(methylsulfonyl)-6-((trimethylsilyl)ethynyl)quinazoline

To the solution of 5-chloro-2-(methylthio)-6-((trimethylsilyl)ethynyl)quinazoline (723 mg, 2.2 mmole) in 20 ml of dichloromethane at 0° c. was added the solution of 3-chloroperbenozic acid (−77% pure, 926 mg, 4.13 mmole) in 10 ml of dichloromethane.

The reaction solution was stirred at room temperature for overnight and was monitored by LC/MS. After completion, the reaction solution was diluted with 200 ml of dichloromethane. The organic phase was washed with saturated NaHCO₃ solution (2×60 ml), water (30 ml) and brine (50 ml), then dried over Na₂SO₄ and evaporated in vacuo to give 5-chloro-2-(methylsulfonyl)-6-((trimethylsilyl)ethynyl)quinazoline as yellowish brown powder (732 mg) that was used in next step without further purification. ES/MS m/z 339.0 (MH⁺).

### Example 1020

### (Table 3): N-(3-methoxyphenyl)-7-(1-methylpiperidin-4-yloxy)-6-(thiazol-2-yl) quinazolin-2-amine

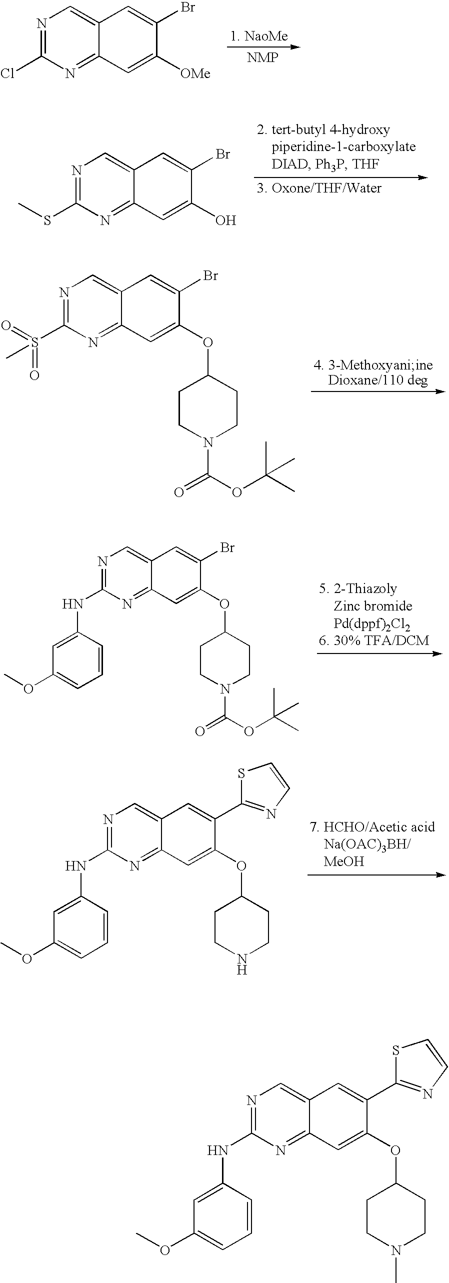

### Step 1. Preparation of 6-Bromo-2-(methylthio) quinazolin-7-ol

To a solution of 6-bromo-2-chloro-7-methoxyquinazoline (1 eq) (See example 11) in NMP (5 ml) was added NaOMe (2 eq). The reaction mixture was heated at 80° C. for 2 h. The reaction mixture was diluted with water and pH was adjusted to 5.5 with 1NHCl. The precipitate was filtered, washed and dried under vacuum to provide title product as a yellow solid (yield, 60%). ES/MS m/z 271.0/273.0 (MH⁺).

2,6-bis(methylthio) quinazolin-7-ol was formed as a side product (40%)

### Step 2. Preparation of tert-butyl-4-(6-bromo-2-(methylthio) quinazolin-7-yloxy)piperidine-1-carboxylate

To a solution of triphenylphosphine (2 eq) in THF was added di-ethylazodicarboxylate (2 eq). The mixture was stirred 15 minutes at ambient temperature under nitrogen atmosphere. To that was added tert-butyl -4-hydroxypiperidine-1-carboxylate (3 eq). The mixture was stirred 15 minutes at ambient temperature followed by addition of 6-bromo-2-(methylthio) quinazolin-7-ol (1 eq). The mixture was stirred overnight at ambient temperature. The reaction mixture was concentrated and purified by flash column chromatography (10% EtOAc/Hexane) to provide product as a white solid in 70% yield. ES/MS m/z 454.0/456.0 (MH⁺).

### Step 3. Preparation of tert-butyl 4-(6-bromo-2-(methyl sulfonyl) quinazolin-7-yloxy)piperidine-1-carboxylate

To a solution of tert-butyl-4-(6-bromo-2-(methylthio) quinazolin-7-yloxy)piperidine-1-carboxylate (1 eq) in THF (10 ml) was added a solution of oxone in water (10 ml) at 0° C. The reaction mixture was stirred for 30 min at 0° C. then warmed to room temperature and stirred overnight. The reaction was cooled to 0° C. and quenched with satd. sodium thiosulfate solution. The product was extracted in ethylacetate. The ethylacetate extracts were combined together, washed with brine and dried over sodium sulfate. Filtered, evaporated and dried under vacuum to provide product in 90% yield. ES/MS m/z 486.0/488.0 (MH⁺). Used for next step without further purification.

### Step 4. Preparation of tert-butyl 4-(6-bromo-2-(3-methoxyphenylamino) quinazolin-7-yloxy)piperidine-1-carboxylate

A solution of tert-butyl 4-(6-bromo-2-(methyl sulfonyl) quinazolin-7-yloxy)piperidine-1-carboxylate (1 eq) and 3-methoxyaniline (2 eq) in dioxane was heated in sealed tube at 110° C. for 24 h. The product was purified by semi-prep HPLC and lyophilyzed to provide pure product as a yellow solid in 50% yield. ES/MS m/z 529.0 /531.0 (MW).

### Step 5. Preparation of tert-butyl 4-(2-(3-methoxyphenylamino)-6-(thiazol-2-yl) quinazolin-7-yloxy)piperidine-1-carboxylate

A mixture of tert-butyl 4-(6-bromo-2-(3-methoxyphenylamino) quinazolin-7-yloxy)piperidine-1-carboxylate (1 eq), 2-thiazolylzincbromide (5 eq, 0.5M solution in THF) and Pd(dppf)₂Cl₂ (0.1 eq) was heated in microwave at 120° C. for 10 min. The reaction mixture was diluted with ethylacetate and washed with water and brine. Dried over sodium sulfate, filtered, evaporated to provide crude product in quantitative yield.

ES/MS m/z 534.2 (MH⁺).

### Step 6. Preparation of N-(3-methoxyphenyl)-7-(piperidin-4-yloxy)-6-(thiazol-2-yl) quinazolin-2-amine

A solution of crude tert-butyl 4-(2-(3-methoxyphenylamino)-6-(thiazol-2-yl) quinazolin-7-yloxy)piperidine-1-carboxylate in 30% TFA/DCM was stirred at room temperature for 30 min. The solvent was evaporated and crude was purified by semi-prep HPLC to provide pure product in 40% yield. ES/MS m/z 434.2 (MH⁺).

### Step 7. Preparation of N-(3-methoxyphenyl)-7-(1-methylpiperidin-4-yloxy)-6-(thiazol-2-yl) quinazolin-2-amine

To a solution of N-(3-methoxyphenyl)-7-(piperidin-4-yloxy)-6-(thiazol-2-yl) quinazolin-2-amine (1 eq) in methanol was added HCHO (10 eq) and a drop of acetic acid. Stirred 10 min at room temperature, followed by the addition of Na(OAc)₃BH (4 eq). The reaction mixture was stirred 1 h/rt. Purified by semi-prep HPLC to provide product as a yellow solid in 70% yield. ES/MS m/z 448.2 (MH⁺).

### Example 1075

### (Table 3): Synthesis of 7-(piperidin-4-yloxy)-N-(3-propoxyphenyl)-6-(thiazol-2-yl) quinazolin-2-amine

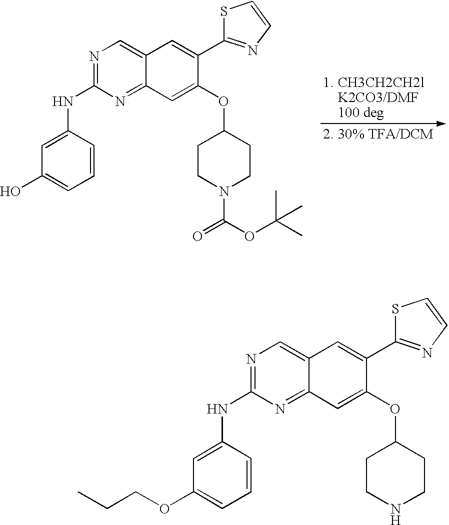

### Step 1. Preparation of tert-butyl-4-(2-(3-propoxyphenylamino)-6-(thiazol-2-yl) quinazolin-7-yloxy)piperidine-1-carboxylate

To tert-butyl-4-(2-(3-hydroxyphenylamino)-6-(thiazol-2-yl) quinazolin-7-yloxy)piperidine-1-carboxylate (1 eq) (For synthesis, see example 21) in DMF (2 ml) was added K₂CO₃ (5 eq) and iodopropane (3 eq). The reaction mixture was heated at 100° C. for 48 h.

The reaction mixture was partitioned between ethylacetate and water. Ethylacetate layer was separated and washed with water, brine and dried over sodium sulfate. Filtered and evaporated to provide crude product. ES/MS m/z 562.2 (MH⁺).

### Step 2. Preparation 7-(piperidin-4-yloxy)-N-(3-propoxyphenyl)-6-(thiazol-2-yl) quinazolin-2-amine

For synthesis, see example 1020, step 6. ES/MS m/z 462.2 (MH⁺).

### Example 1082

### (Table 3): 7-(1-(2-fluoromethylpiperidin-4-yloxy)-N-(3-fluorophenyl)-6-(thiazol-2-yl) quinazolin-2-amine

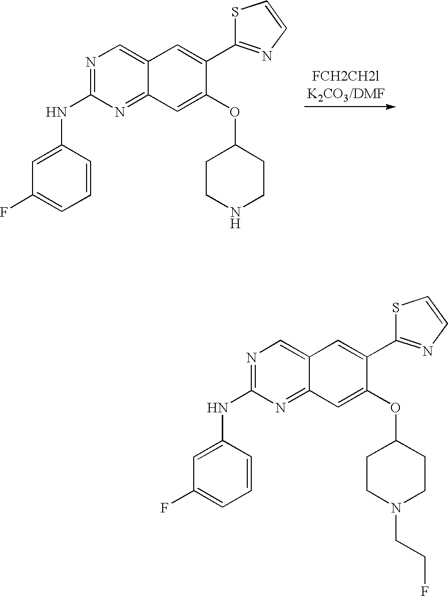

To N-(3-fluorophenyl)-7-(piperidin-4-yloxy)-6-(thiazol-2-yl) quinazolin-2-amine (1 eq) (For synthesis, see example 11) in DMF was added K₂CO₃ (5 eq) and 1-fluoro-3-iodoethane (1.2 eq). The reaction mixture was stirred overnight at rt. Product was purified by semi-prep HPLC. ES/MS m/z 468.2 (MH⁺).

### Example 1084

### (Table 3) Synthesis of N-(3-fluorophenyl)-7-(1-methylpiperidin-4-yloxy)-6-(thiazol-4-yl) quinazolin-2-amine

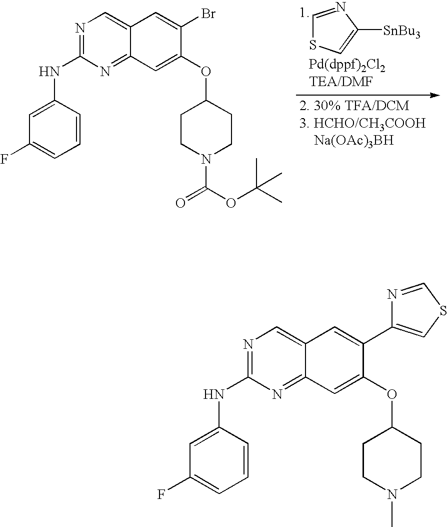

### Step 1. Preparation of tert-butyl 4-(2-(3-fluorophenylamino)-6-(thiazol-4-yl) quinazolin-7-yloxy)piperidin-1-carboxylate

A mixture of tert-butyl-4-(6-bromo-2-(3-fluorophenylamino) quinazolin-7-yloxy)piperidine-1-carboxylate (1 eq, see example 11 for synthesis), 4-tributyltin thiazole (3 eq), TEA (3.5 eq) and Pd (dppf)₂Cl₂ (0.1 eq) in DMF (1.5 ml) was heated in microwave at 120° C. for 10 min. The reaction mixture was partitioned between ethylacetate and water. Ethylacetate layer was separated and washed with water, brine and dried over sodium sulfate. Filtered, evaporated and purified by semi-prep HPLC to provide pure product in 50% yield ES/MS m/z 522.1 (MH⁺).

### Step 2. Preparation of N-(3-fluorophenyl)-7-(piperidin-4-yloxy)-6-(thiazol-4-yl) quinazolin-2-amine

For synthesis see example 21, step 6. ES/MS m/z 422.1 (MH⁺).

### Step 3. Preparation of N-(3-fluorophenyl)-7-(1-methylpiperidin-4-yloxy)-6-(thiazol-4-yl) quinazolin-2-amine

For synthesis see example 1020, step 7. ES/MS m/z 436.1 (MH⁺).

### Example 1066

### (Table 3)

### Synthesis of N-(3-fluorophenyl)-6-(isoxazol-4-yl)-7-(piperidin-4-yloxy) quinazolin-2-amine

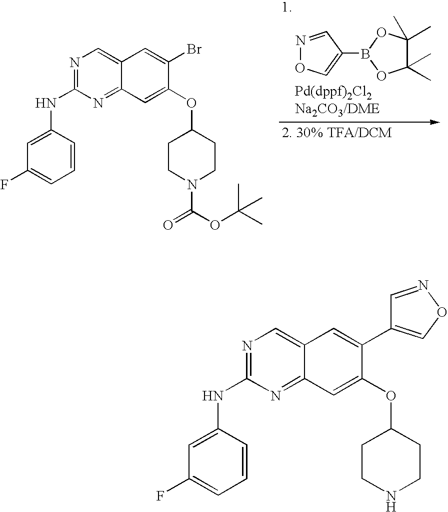

### Step 1. Preparation of tert-butyl 4-(2-(3-fluorophenylamino)-6-(isoxazol-4-yl) quinazolin-7-yloxy)piperidin-1-carboxylate

A mixture of tert-butyl-4-(6-bromo-2-(3-fluorophenylamino) quinazolin-7-yloxy)piperidine-1-carboxylate (1 eq, see example 11 for synthesis), 4-isoxazole boronic ester (3 eq), 1,1′-bis(diphenylphosphino)ferrocenedichloro palladium(II) (0.1 eq), sodium carbonate (0.5 ml of 2M aqueous solution) in DME (2 ml) was heated in microwave at 120° C. for 10 min. The reaction mixture was partitioned between ethylacetate and water. Ethylacetate layer was separated and washed with water, brine and dried over sodium sulfate. Filtered, evaporated and purified by semi-prep HPLC to provide pure product in 50% yield ES/MS m/z 506.1 (MH⁺).

Note: Some debrominated product was also observed in above reaction

### Step 2. Preparation of N-(3-fluorophenyl)-6-(isoxazol-4-yl) quinazolin-2-amine

For synthesis see example 1020, step 6. ES/MS m/z 406.1 (MH⁺).

### Example 1135

### (Table 3)

### Synthesis of 7-(piperidin-4-yloxy)-N-(3-(pyridine-3-yl)phenyl)-6-(thiazol-2-yl) quinazolin-2-amine

The subject compound was prepared according to the general Scheme below:

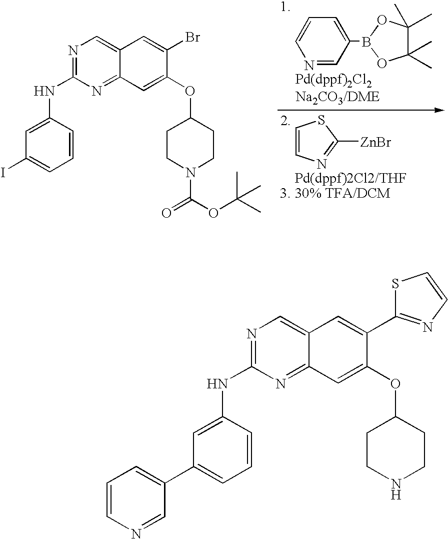

### Step 1. Preparation of tent-butyl 4-(6-bromo-2-(3-(pyridine-3-yl) phenylamino) quinazolin-7-yloxy)piperidin-1-carboxylate

A mixture of tert-butyl-4-(6-bromo-2-(3-iodophenylamino) quinazolin-7-yloxy)piperidine-1-carboxylate (1 eq, see example 11 for synthesis), 3-pyridine boronic ester (1.2 eq), 1,1′-bis(diphenylphosphino)ferrocenedichloro palladium(II) (0.1 eq), sodium carbonate (0.5 ml of 2M aqueous solution) in DME (2 ml) was heated in microwave at 120° C. for 10 min. The reaction mixture was partitioned between ethylacetate and water. Ethylacetate layer was separated and washed with water, brine and dried over sodium sulfate. Filtered, evaporated and purified by semi-prep HPLC to provide pure product in 50% yield ES/MS m/z 576.2/578.2.1 (MH⁺).

Note: Some bis-substituted product was also isolated in above reaction

### Step 2. Preparation of tert-butyl 4-(2-(3-(pyridine-3-yl)phenylamino)-6-(thiazol-2-yl) quinazolin-7-yloxy)piperidin-1-carboxylate

For Synthesis, see example 1020, step 5. ES/MS m/z 581.2 (MH⁺).

### Step 3. Preparation of 7-(piperidin-4-yloxy)-N-(3-(pyridine-3-yl) phenyl)-6-(thiazol-2-yl) quinazolin-2-amine

For Synthesis, see example 1020, step 6. ES/MS m/z 481.2 (MH⁺).

### Example 1117

### (Table 3)

### Synthesis of (5-(2-(3-(oxazol-5-yl)phenylamino (7-(piperidin-4-yloxy) quinazolin-6-yl)-1H-1,2,3-triazol-4-yl)methanol

The subject compound was prepared according to the general Scheme below:

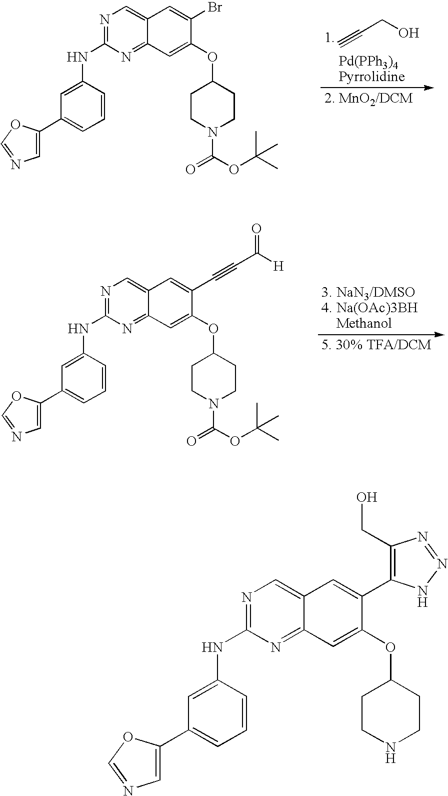

### Step 1. Preparation of tert-butyl 4-(6-(3-hydroxyprop-1-ynyl)-2-(3-(oxazol-5-yl) phenylamino)quinazolin-7-yloxy)piperidin-1-carboxylate

To tert-butyl 4-(6-bromo-2-(3-(oxazol-5-yl)phenylamino)quinazolin-7-yloxy)piperidin-1-carboxylate (1 eq) and tetrakis (PPH₃)Pd(0) (0.02 eq) in pyrrolidine (1.5 ml) at room temperature was added propargyl alcohol (2 eq, in 1 ml of pyrrolidine). The reaction mixture was heated in sealed tube at 80° C. for 1 h. The reaction was quenched with satd. NH₄Cl and product was extracted with diethyl ether. Ether extracts were washed with brine, dried over sodium sulfate, filtered and evaporated. Purified by semi-prep HPLC to provide pure product as a yellow solid in 40% yield. ES/MS m/z 542.2 (MH⁺).

### Step 2. Preparation of tert-butyl 4-(2-(3-(oxazol-5-yl) phenylamino)-6-(3-oxoprop-1-ynyl) quinazolin-7-yloxy)piperidin-1-carboxylate

A mixture of tert-butyl 4-(6-(3-hydroxyprop-1-ynyl)-2-(3-(oxazol-5-yl) phenylamino) quinazolin-7-yloxy)piperidin-1-carboxylate (1 eq) and MnO₂ (5 eq) in DCM (5 ml) was stirred overnight at rt. The product was filtered through celite and solvent was evaporated to provide pure product in 90% yield. ES/MS m/z 540.3 (MH⁺).

### Step 3. Preparation of tent-butyl 4-(6-(4-formyl-1H-1,2,3-triazol-5-yl)-2-(3-(oxazol-5-yl)phenylamino) quinazolin-7-yloxy)piperidin-1-carboxylate

To a stirred solution of sodium azide (1.2 eq) in DMSO (2 ml) in ice-water bath was added tert-butyl 4-(2-(3-(oxazol-5-yl) phenylamino)-6-(3-oxoprop-1-ynyl) quinazolin-7-yloxy)piperidin-1-carboxylate in DMSO (1 ml). The reaction mixture was stirred 30 min at room temperature then poured to vigorously stirred biphasic solution of 5% KH₂PO₄ and diethyl ether. Ether layer was separated, washed with brine and dried over sodium sulfate. Filtered, evaporated and dried under vacuum to provide product as a white solid in 70% yield. ES/MS m/z 583.31 (MH⁺).

### Step 4. Preparation of tert-butyl 4-(6-(4-(hydroxymethyl)-1H-1,2,3-triazol-5-yl)-2-(3-(oxazol-5-yl)phenylamino) quinazolin-7-yloxy)piperidin-1-carboxylate

To tert-butyl 4-(6-(4-formyl-1H-1,2,3-triazol-5-yl)-2-(3-(oxazol-5-yl) phenylamino) quinazolin-7-yloxy)piperidin-1-carboxylate (1 eq) in methanol was added Na(OAc)₃BH (5 eq). The reaction mixture was stirred 1 h at room temperature. Purified by semi-prep HPLC to provide pure product as a yellow solid in 40% yield. ES/MS m/z 584.2 (MH⁺).

### Step 5. Preparation of (5-(2-(3-(oxazol-5-yl)phenylamino (7-(piperidin-4-yloxy) quinazolin-6-yl)-1H-1,2,3-triazol-4-yl)methanol

For Synthesis, see example 1020, step 6. ES/MS m/z 484.2 (MH⁺).

### Example 1140

### (Table 3): (2R,4S)-4-(2-(3-fluorophenylamino)-6-(thiazol-2-yl)quinazolin-7-yloxy)piperidine-2-carboxamide

### Synthesis of 4-((tert-butyldimethylsilyloxy)methyl)-2-(tributylstannyl)thiazole

The subject compound was prepared according to the general Scheme below:

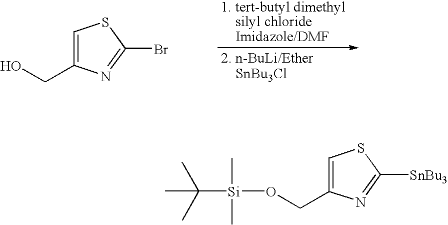

### Step 1. Preparation of 2-bromo-4-(tert-butylsilyloxy)methyl)thiazole

A mixture of 2-bromothiazol-4-yl)methanol (1 eq), imidazole (4 eq) and tert-butyl-dimethylsilylchloride (2 eq) in DMF(10 ml) was stirred 2 h at room temperature. The reaction mixture was partitioned between ethylacetate and water. Ethylacetate layer was separated and washed with water, brine and dried over sodium sulfate. Filtered, evaporated and purified by flash chromatography (10% EtOAc/Hexane) to provide pure product as a colorless liquid in 95% yield ES/MS m/z 307.9/309.9 (MH⁺).

### Step 2. Preparation of 4-((tert-butyldimethylsilyloxy)methyl)-2-(tributylstannyl)thiazole

To flame dried flask under nitrogen at −78° C. was added anhydrous ether (10 ml) and n-butyl lithium (1.5 eq, 2.5M solution in hexane) followed by addition of 2-bromo-4-(tert-butylsilyloxy)methyl) thiazole solution in ether (1 eq, 3 ml). Stirred at −78° C. for 1 h. Then a solution of tributyltinchloride in ether (1.5 eq, 1 ml) was added dropwise. Stirred at this temperature additional 1 h. The reaction mixture was quenched with satd. sodium bicarbonate and compound was extracted in ether. Ether extracts were combined, washed with brine and dried over sodium sulfate. Filtered, evaporated and dried under vacuum to provide product as a light yellow liquid. Used without further purification.

### Synthesis of (2R,4S)-4-(2-(3-fluorophenylamino)-6-(thiazol-2-yl)quinazolin-7-yloxy)piperidine-2-carboxamide

The subject compound was prepared according to the general Scheme below:

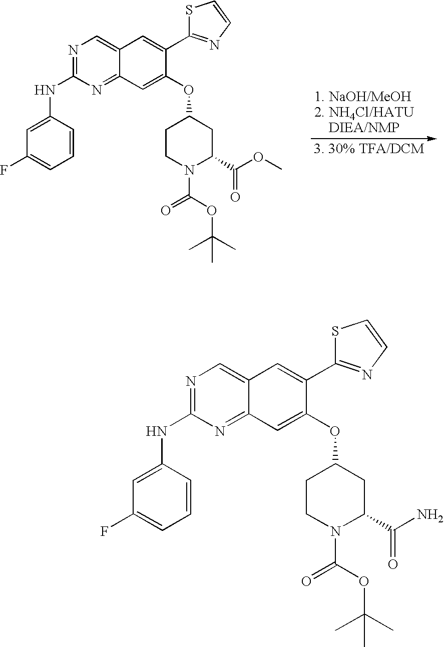

### Step 1. Preparation of (2R,4S)-1-(tert-butoxycarbonyl)-4-(2-(3-fluorophenylamino)-6-(thiazol-2-yl)quinazolin-7-yloxy)piperidine-2-carboxylic acid

To (2R,4S)-1-tert-butyl -2-methyl-4-(2-(3-fluorophenylamino)-6-(thiazol-2-yl)quinazolin-7-yloxy)piperidine-1,2-dicarboxylate (1 eq) in methanol (3 ml) was added aqueous solution of sodium hydroxide (5 eq). The reaction mixture was stirred overnight at rt. The solvent was evaporated; residue was taken in water and made acidic with 1NHCl. Product was extracted in ethylacetate. The extracts were combined, washed with brine and dried over sodium sulfate. Filtered, evaporated and dried under vacuum to provide product as a light brown solid in 70% yield. ES/MS m/z 566.1 (MH⁺).

### Step 2. Preparation of (2R,4S)-tert-butyl-2-carbamoyl-4-(2-(3-fluorophenylamino)-6-(thiazol-2-yl)quinazolin-7-yloxy)piperidine-1-carboxylate

A mixture of (2R,4S)-1-(tert-butoxycarbonyl)-4-(2-(3-fluorophenylamino)-6-(thiazol-2-yl)quinazolin-7-yloxy)piperidine-2-carboxylic acid (leg), NH₄Cl (10 eq), HATU(1.75 eq) and DIEA (5 eq) in NMP (2 ml) was stirred overnight at rt. The product was purified by semi-prep HPLC. ES/MS m/z 565.1 (MH⁺).

### Step 3. Preparation of (2R,4S)-4-(2-(3-fluorophenylamino)-6-(thiazol-2-yl)quinazolin-7-yloxy)piperidine-2-carboxamide

For Synthesis, see example 21, step 6. ES/MS m/z 465.1 (MH⁺).

### Examples 1161, 1165-1171, and 1174

The subject compounds were prepared according to the general Scheme below:

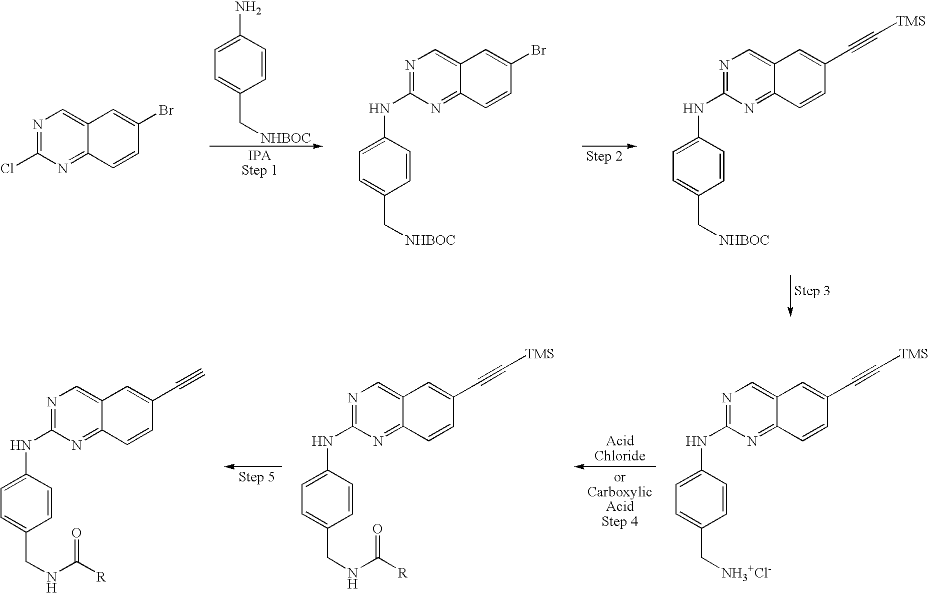

### Step 1:

This reaction was conducted in a similar manner to that described in Example 9, step 5.

### Step 2:

This reaction was conducted in a similar manner to that described in Example 10, step 1.

### Step 3:

Boc Amine was dissolved in Dioxane and treated with 4 M HCl/Dioxane (20 eq). Reaction stirred at room temperature for 2 hours. Reaction was concentrated to a white solid identified as desired product.

### Step 4:

### Method A

Amine was dissolved in DMF and Et₃N (3 eq) at room temperature and treated with appropriate acid chloride (1.5 eq). Reaction stirred at room temperature for 10 hours and was directly purified by RPHPLC to yield desired compounds.

### Method B

Amine, HOAT (1.5 eq), HATU (1.5 eq) and Et₃N (3.0 eq) were combined in DMF. Corresponding carboxylic acid (1.2 eq) was added and reactions stirred at room temperature for 10 hours and was directly purified by RPHPLC to yield desired compounds

### Step 5:

This reaction was conducted in a similar manner to that described in Example 10, step 2.

### Examples 1153-1155, 1157, 1158, 1160, 1162-1164, 1172, and 1173

The subject compounds were prepared according to the general Scheme below:

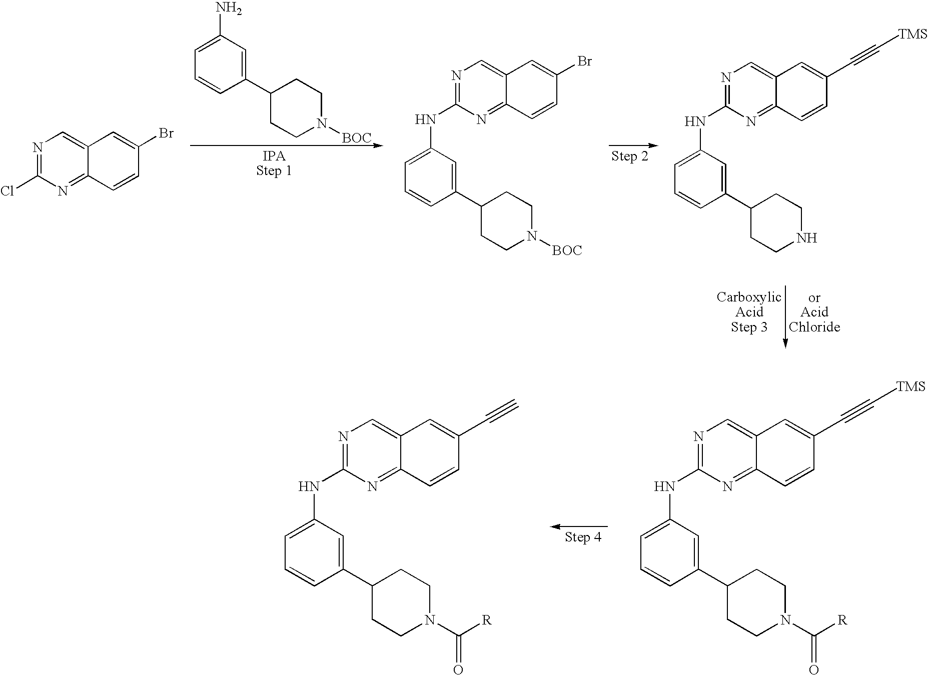

### Step 1:

This reaction was conducted in a similar manner to that described in Example 9, step 5.

### Step 2:

This reaction was conducted in a similar manner to that described in Example 10, step 1.

### Step 3:

### Method A

Amine was dissolved in DMF and Et₃N (3 eq) at room temperature and treated with appropriate acid chloride (1.5 eq). Reactions stirred at room temperature for 10 hours and was directly purified by RPHPLC to yield desired compounds

### Method B

Amine, HOAT (1.5 eq), HATU (1.5 eq) and Et₃N (3.0 eq) were combined in DMF. Corresponding carboxylic acid (1.2 eq) was added and reactions stirred at room temp for 10 hours and was directly purified by RPHPLC to yield desired compounds.

### Step 4:

This reaction was conducted in a similar manner to that described in Example 10, step 2.

### Examples 1156 and 1159

The subject compounds were prepared according to the general Scheme below:

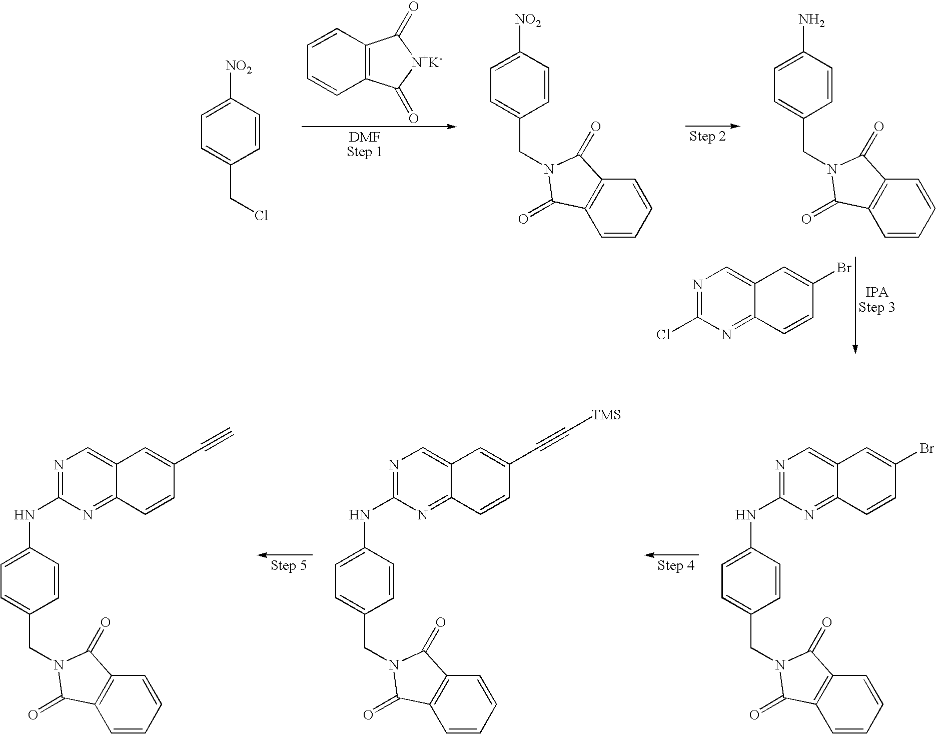

### Step 1:

Chloride and potassium phthalamide (1.5 eq) were combined in DMF and heated at 120° C. for 10 minutes in the microwave. Reaction was concentrated and purified on silica with a 0 to 100% EtOAc/Hexane gradient to provide desired compound as a white solid.

### Step 2:

This reaction was conducted in a similar manner to that described in Example 459, step 1.

### Step 3:

This reaction was conducted in a similar manner to that described in Example 9, step 5.

### Step 4:

This reaction was conducted in a similar manner to that described in Example 10, step 1.

### Step 5:

This reaction was conducted in a similar manner to that described in Example 10, step 2.

### Example 1175

The subject compound was prepared according to the general Scheme below:

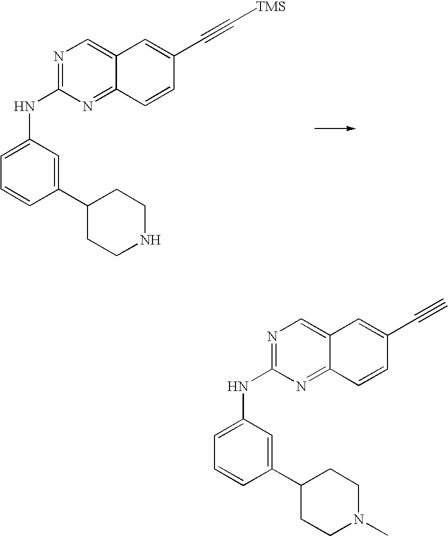

Amine (from Example 1153 or 1154) and formaldehyde (5.0 eq of 37% aq. Solution) were suspended in CH₂Cl₂ at room temperature. Sodium triacetoxyborohydride was added at once and reaction stirred for 10 hours. After this time, reaction was quenched by addition of water followed by concentration and purification by RPHPLC. Purified fractions were combined and treated as per Example 10, step 2 to yield desired compound.

### Example 1176

The subject compound was prepared according to the general Scheme below:

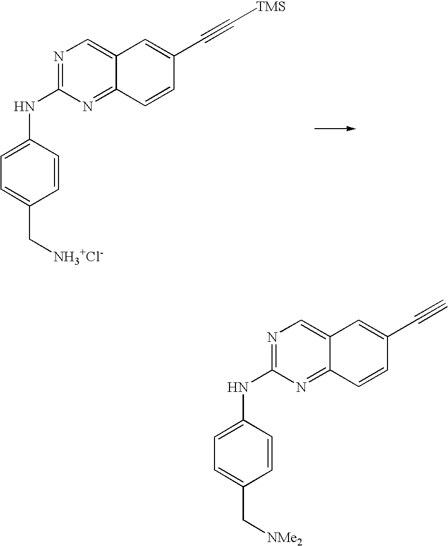

Amine (from Example 1161 or 1165) and formaldehyde (5.0 eq of 37% aq. Solution) were suspended in CH₂Cl₂ at room temperature. Sodium triacetoxyborohydride was added at once and reaction stirred for 10 hours. After this time, reaction was quenched by addition of water followed by concentration and purification by RPHPLC. Purified fractions were combined and treated as per Example 10, step 2 to yield desired compound.

### Example 1151

The subject compound was prepared according to the general Scheme below:

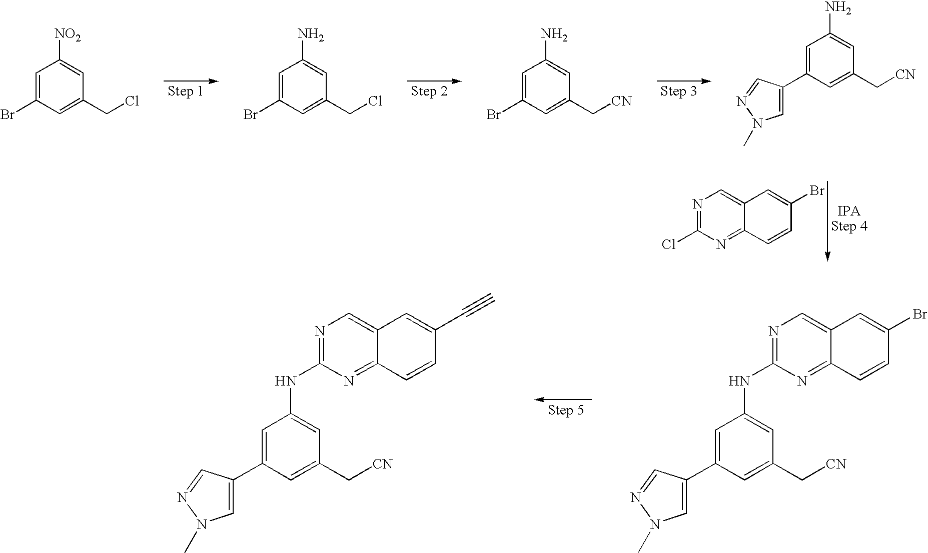

### Step 1:

Nitro and SnCl₂—(H₂O)₂ (10 eq) were combined in a solution of isopropanol and 12 N HCl. Reaction was heated at 110° C. for 2 hours and was concentrated to 25% original volume. Resulting suspension was dissolved in a 3:1 mix of EtOAc:CH₂Cl₂ and slowly neutralized with NaHCO₃ (sat. aq.) to pH 7. Resulting mixture was filtered through a Celite plug and the organic layers were separated. The aqueous layer was extracted 3× with CH₂Cl₂ and the combined organics were dried and concentrated to provide 5.7 g of yellow solid which was used without further purification.

### Step 2:

To a solution of amine in DMSO was added NaCN (2.0 eq) at room temperature. Reaction stirred for 1 hour and was quenched with water and extracted 2× with EtOAc. Combined organics were dried over MgSO₄ and concentrated to a yellow oil. Silica gel purification with a 0 to 80% gradient provided the desired product as a white solid.

### Step 3:

This reaction was conducted in a similar manner to that described in Example 469, step 2.

### Step 4:

This reaction was conducted in a similar manner to that described in Example 9, step 5.

### Step 5:

This conversion was achieved in a similar manner to that described in Example 10, step 1 and step 2.

### Example 1177

The subject compound was prepared according to the general Scheme below:

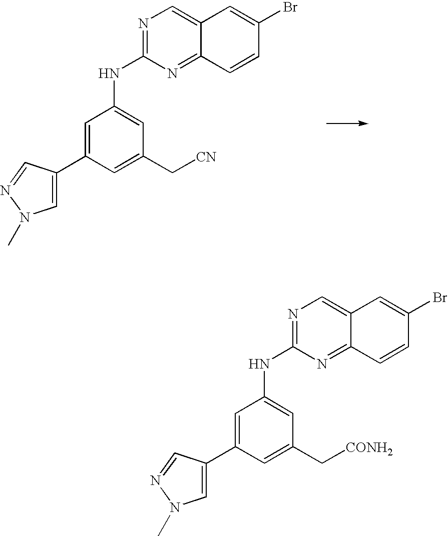

Nitrile (from Example 1151) was treated with 1N NaOH (10 eq) in microwave at 120° C. for 10 minutes. Reaction was purified directly by RPHPLC to provide desired product.

### Example 1178

The subject compound was prepared according to the general Scheme below:

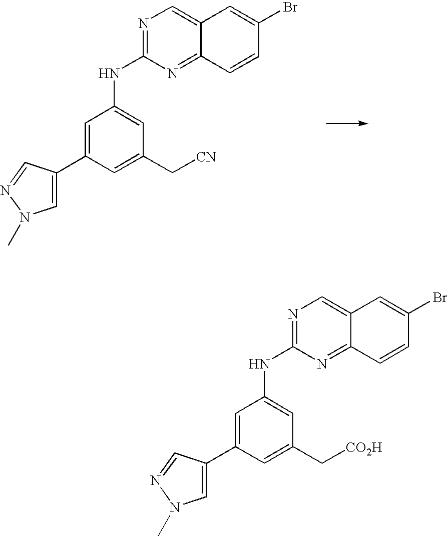

Nitrile (from Example 1151) was treated with 1N NaOH (10 eq) in sealed glass bomb at 110° C. for 12 hours. Reaction was cooled to room temperature and concentrated to 20% original volume. 1N HCl was added to pH 7 and resulting yellow solid was collected and dried to provide desired product.

### Examples 1179-1184

The subject compound was prepared according to the general Scheme below:

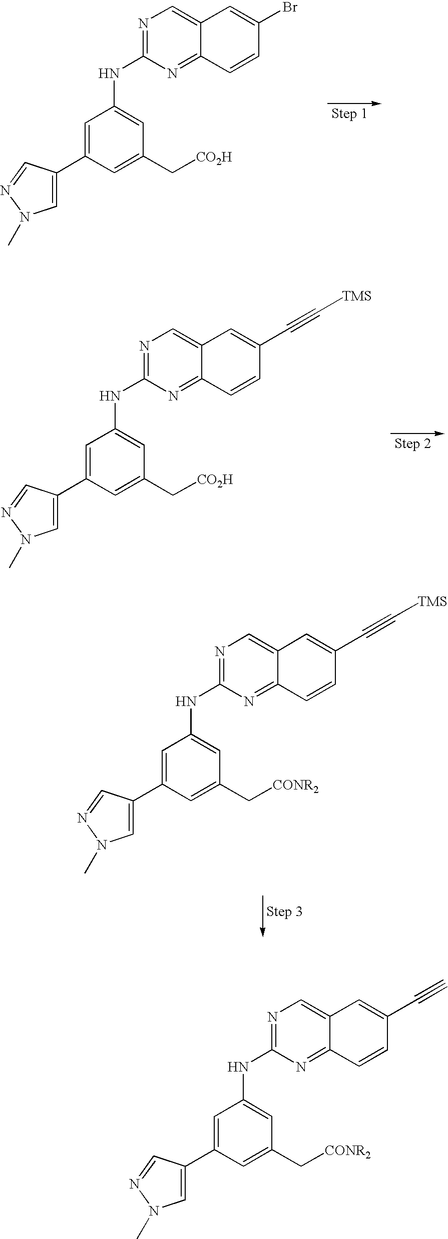

### Step 1:

This reaction was conducted in a similar manner to that described in Example 10, step 1 using acid from Example 1178.

### Step 2:

Acid, HOAT (1.5 eq), HATU (1.5 eq) and Et₃N (3.0 eq) were combined in DMF. Corresponding amine was added and reactions stirred at room temp for 10 hours and was directly purified by RPHPLC to yield desired compounds.

### Step 3:

This reaction was conducted in a similar manner to that described in Example 10, step 2.

### Example 1150

The subject compound was prepared according to the general Scheme below:

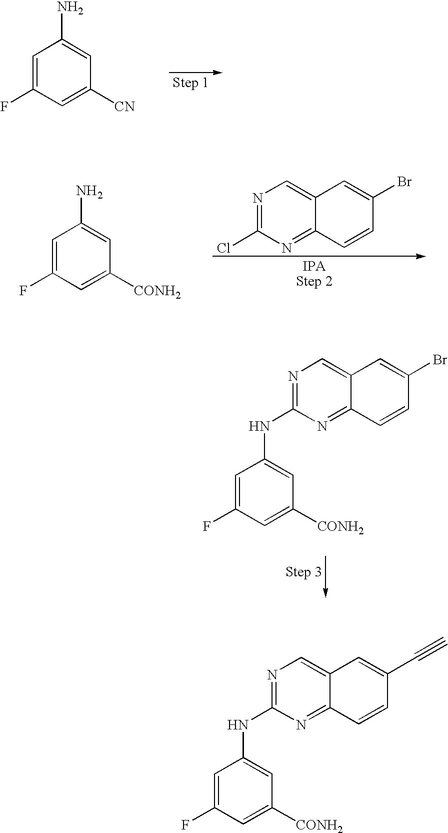

### Step 1:

Amine was suspended in NH₄OH and heated at 130° C. for 15 minutes in microwave. Reaction was concentrated to a yellow solid which was used without further purification.

### Step 2:

This reaction was conducted in a similar manner to that described in Example 9, step 5.

### Step 3:

This conversion was achieved in a similar manner to that described in Example 10, step 1 and step 2.

A number of embodiments of the invention have been described. Nevertheless, it will be understood that various modifications may be made without departing from the spirit and scope of the invention. Such modifications are also intended to fall within the scope of the appended claims. Each reference, including all patents, patent applications, publications, and gene bank sequences cited in the present application, is incorporated herein by reference in its entirety.

What is claimed is:

1. A compound of Formula (Ia): 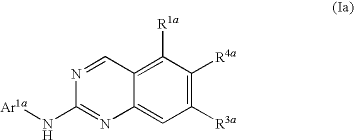
or pharmaceutically acceptable salt thereof, wherein:
R^{1a} is selected from H and halogen;
R^{3a} is selected from C₂₋₆ heterocycloalkyloxy and C₁₋₆ heteroaryl-C₁₋₄-alkoxy; wherein said C₂₋₆ heterocycloalkyloxy and C₁₋₆ heteroaryl-C₁₋₄-alkoxy are each optionally substituted by a R^{w} group;
R^{4a} is selected from H, a thiazole ring, a pyrazole ring, a triazole ring, a tetrazole ring, a pyridine ring, C₃₋₆ cycloalkyl, cyano, halogen, C₂₋₆ alkynyl, C₁₋₆ heteroaryl-C₁₋₄-alkyl, C₁₋₆ heteroaryl-C₁₋₄-alkynyl, —C(═O)R^{a}, and —C(═O)NR^{b}R^{c}; wherein said thiazole ring, pyrazole ring, triazole ring, tetrazole ring, pyridine ring, C₁₋₆ heteroaryl-C₁₋₄-alkyl and C₁₋₆ heteroaryl-C₁₋₄-alkynyl are each optionally substituted by 1 or 2 independently selected R^{x″} groups;
Ar^{1a} is selected from phenyl optionally substituted at the meta position by one R^{y′} or, alternatively, at the para position by one R^{y″} group;
each R^{w} is independently selected from halogen, C₁₋₆ alkyl, amino, C₁₋₆ alkylamino, di-C₁₋₄-alkylamino, C₁₋₆ alkoxycarbonyl, and C₁₋₆ alkylcarbamyl; wherein said C₁₋₆ alkyl and C₁₋₆ alkylamino are each optionally substituted by a group selected from hydroxyl, C₁₋₆ alkoxy, amino, C₁₋₆ alkylamino, and di-C₁₋₄-alkylamino;
each R^{x″} is independently selected from halogen, hydroxyl, C₁₋₆ alkyl, C₁₋₆ alkoxycarbonyl, and carbamyl; wherein said C₁₋₆ alkyl and C₁₋₆ alkoxycarbonyl are each optionally substituted by a group selected from hydroxyl, amino, C₁₋₄ alkylamino, and aminosulfonyl;
R^{y′} is selected from halogen, C₁₋₆ alkoxy, C₁₋₆ alkyl, carbamyl, aminosulfonyl, and C₁₋₆ alkylsulfonylamino; wherein said C₁₋₆ alkyl and C₁₋₆ alkoxy are each substituted by 1 or 2 groups independently selected from hydroxyl, amino, C₁₋₄ alkylamino, and aminosulfonyl;
R^{y″} is selected from C₁₋₆ haloalkyl;
R^{a} is selected from H, C₁₋₆ alkoxy and C₂₋₆ heterocycloalkyl;
R^{b} is selected from H and C₁₋₆ alkyl; and
R^{c} is selected from C₁₋₆ alkyl, C₃₋₆ cycloalkyl, C₁₋₆ heteroaryl, C₂₋₆ heterocycloalkyl, C₂₋₆ heterocycloalkyl-C₁₋₄-alkyl, and C₁₋₆ heteroaryl-C₁₋₄-alkyl; wherein said C₁₋₆ alkyl is optionally substituted by a group selected from hydroxyl and C₁₋₄ alkoxy;
provided that:
(a) when R^{3a} is piperidin-4-yloxy or N-methylpiperidin-4-yloxy, then R⁴ is not selected from thiazol-2-yl, thiazol-4-yl, thiazol-5-yl, 5-methylthiazol-2-yl, 5-(hydroxymethyl)thiazol-2-yl, 5-(hydroxymethyl)thiazol-4-yl, pyridin-2-yl, pyridin-4-yl, pyridin-3-yl, 4-methylpyridin-3-yl, 5-chloropyridin-4-yl, 3-methylpyridin-2-yl, and 1-methylpyrazol-5-yl;
(b) when R^{4a} is ethynyl and Ar^{1a} is 3-fluorophenyl, then R³ is not selected from pyridin-3-ylmethoxy, 6-chloropyridin-3-ylmethoxy, pyridin-2-ylmethoxy, pyridin-4-ylmethoxy, 1-(pyridin-4-yl)ethoxy, 6-methoxypyridin-2-ylmethoxy, thiazol-5-ylmethoxy, pyrazin-2-ylmethoxy, 5-methylisooxazol-3-yl, azetidin-3-yloxy, N-methylazetidin-3-yloxy, N-isopropylazetidin-3-yloxy, pyrrolidin-3-yloxy, N-methyl, and pyrrolidin-3-yloxy;
(c) when R^{4a} is ethynyl, then Ar^{1a} is not selected from 3-(2-hydroxypropan-2-yl)phenyl, 3-(1-hydroxyethyl)phenyl, 3-amino sulfonylphenyl, phenyl, 3-(methylsulfonylamino)phenyl, 3-(N,N-dimethylaminomethyl)phenyl, 3-aminosulfonylphenyl, and 3-carbamylphenyl;
(d) when R^{4a} is thiazol-2-yl, then Ar^{1a} is not selected from 3-(methylaminosulfonyl)phenyl, 3-chlorophenyl, 3-carbamylphenyl, 3-fluorophenyl, 2-(hydroxypropan-2-yl)phenyl, 1-hydroxyethyl, 3-aminosulfonylphenyl, and phenyl;
(e) when R^{4a} is thiazol-2-yl and Ar^{1a} is 3-fluorophenyl, then R³ is not selected from pyridin-3-ylmethoxy, 6-chloropyridin-3-ylmethoxy, 2-chloropyridin-4-ylmethoxy, 5-methylisooxazol-3-yl, azetidin-3-yloxy, N-methylazetidin-3-yloxy, N-isopropylazetidin-3-yloxy, pyrrolidin-3-yloxy, N-methyl, pyrrolidin-3-yloxy, piperidin-4-yloxy, and N-methylpiperidin-4-yloxy;
(f) when R^{4a} is bromo, then Ar^{1a} is not selected from phenyl, 3-fluorophenyl, 2-(hydroxypropan-2-yl)phenyl, 1-hydroxyethyl, and 3-amino sulfonylphenyl ;
(g) when R^{4a} is cyano, then R^{3a} is not selected from pyridin-4-ylmethoxy, pyridin-2-ylmethoxy, and 6-methoxypyridin-2-ylmethoxy;
(h) when R^{4a} is cyclopropyl, then R^{3a} is not selected from azetidin-3-yloxy; and
(i) when R^{4a} is hydrogen, then Ar^{1a} is 3-(aminosulfonyl)phenyl.

2. The compound according to claim 1, or pharmaceutically acceptable salt thereof, wherein Ar^{1a} is selected from 3-fluorophenyl, 3-methylaminosulfonyl, 3-carbamylphenyl, 3-chlorophenyl, 4-difluoromethylphenyl, -(2-amino-n-propyl)methylphenyl, 3-(1-amino-3-hydroxy-n-propyl)phenyl, 3-(2-aminoethyl)phenyl, 3-(aminomethyl)phenyl, 3-(3-amino-1-hydroxy)phenyl, 3-(2-hydroxyethyl)phenyl, 3-(hydroxymethyl)phenyl, 3-methylaminosulfonyl, 3-(aminosulfonylmethyl)phenyl, 3-carbamylphenyl and 3-chlorophenyl.

3. The compound according to any one of claims 1 and 2, or pharmaceutically acceptable salt thereof, wherein R^{3a} is selected from piperidin-4-yloxy, N-methylpiperidin-4-yloxy, N-(tert-butoxycarbonyl)piperidin-4-yloxy, 3-fluoropiperidin-4-yloxy, 2-(hydroxymethyl)piperidin-4-yloxy, N-(tert-butoxycarbonyl)piperidin-4-yl, 2-(N-methylcarbamyl)piperidin-4-yloxy, 6-methoxypyridin-3-ylmethoxy, 6-(N-methylamino)pyridin-3-ylmethoxy, 6-(N,N-dimethylamino)pyridin-3-ylmethoxy, 6-(N-(2-hydroxyethyl)amino)pyridin-3-ylmethoxy, 6-(N-(2-methoxyethyl)amino)pyridin-3-ylmethoxy, 6-(N-(2-aminoethyl)amino)pyridin-3-ylmethoxy, and pyridin-3-ylmethoxy; and
R^{4a} is selected from ethynyl, bromo, cyano, cyclopropyl, thiazol-2-yl, pyridin-3-yl, 4-(hydroxymethyl)thiazol-2-yl, 1,2,3-triazol-5-yl, tetrazol-5-yl, pyrazol-2-yl, 5-methylpyrazol-2-yl, 2-(pyridin-2-yl)ethynyl, 2-(pyridin-2-yl)ethyl, 2-(pyridin-3-yl)ethynyl, 2-(pyridin-3-yl)ethyl, 4-(methoxycarbonyl)thiazol-2-yl, 4-carbamylthiazol-2-yl, —C(═O)R^{a}, and —C(═O)NR^{b}R^{c}.

4. The compound according to claim 1, or pharmaceutically acceptable salt thereof, wherein:
R^{1a} is selected from H;
Ar^{1a} is 3-fluorophenyl;
R^{3a} is selected from 3-fluoropiperidin-4-yloxy, 2-(hydroxymethyl)piperidin-4-yloxy, N-(tert-butoxycarbonyl)piperidin-4-yl, and 2-(N-methylcarbamyl)piperidin-4-yloxy; and
R^{4a} is selected from thiazol-2-yl and pyridin-3-yl.

5. The compound according to claim 1, or pharmaceutically acceptable salt thereof, wherein:
R^{1a} is selected from H;
Ar^{1a} is 3-fluorophenyl;
R^{3a} is selected from 6-methoxypyridin-3-ylmethoxy, 6-(N-methylamino)pyridin-3-ylmethoxy, 6-(N,N-dimethylamino)pyridin-3-ylmethoxy, 6-(N-(2-hydroxyethyl)amino)pyridin-3-ylmethoxy, 6-(N-(2-methoxyethyl)amino)pyridin-3-ylmethoxy, and 6-(N-(2-aminoethyl)amino)pyridin-3-ylmethoxy; and
R^{4a} is ethynyl.

6. The compound according to claim 1, or pharmaceutically acceptable salt thereof, wherein:
R^{1a} is selected from H;
Ar^{1a} is selected from 3-(2-amino-n-propyl)methylphenyl, 3-(1-amino-3-hydroxy-n-propyl)phenyl, 3-(2-aminoethyl)phenyl, 3-(aminomethyl)phenyl, 3-(3-amino-1-hydroxy)phenyl, 3-(2-hydroxyethyl)phenyl, and 3-(hydroxymethyl)phenyl;
R^{3a} is selected from pyridin-3-ylmethoxy; and
R^{4a} is selected from ethynyl and bromo.

7. The compound according to claim 1, or pharmaceutically acceptable salt thereof, wherein:
R^{1a} is selected from H;
Ar^{1a} is selected from 3-methylaminosulfonyl, 3-(aminosulfonylmethyl)phenyl, and phenyl;
R^{3a} is selected from piperidin-4-yloxy; and
R^{4a} is thiazol-2-yl and 4-(hydroxymethyl)thiazol-2-yl.

8. The compound according to claim 1, or pharmaceutically acceptable salt thereof, wherein:
R^{1a} is selected from H;
Ar^{1a} is 3-fluorophenyl
R^{3a} is selected from piperidin-4-yloxy and N-(tert-butoxycarbonyl)piperidin-4-yloxy;
R^{4a} is selected from —C(═O)R^{a}, and —C(═O)NR^{b}R^{c};
R^{a} is selected from H, methoxy and morpholin-4-yl;
R^{b} is selected from H and methyl; and
R^{c} is selected from methyl, 2-hydroxyethyl, 2-methoxyethyl, tetrahydro-2H-pyran, tetrahydropyran-2H-methyl, 2-oxopyrrolidinylethyl, and pyridin-3-ylmethyl;

9. The compound according to claim 1, or pharmaceutically acceptable salt thereof, wherein:
R^{1a} is selected from H;
Ar^{1a} is 3-carbamylphenyl and 3-chlorophenyl;
R^{3a} is selected from pyridin-4-ylmethoxy; and
R^{4a} is selected from ethynyl and bromo.

10. A compound of Formula (IIa): 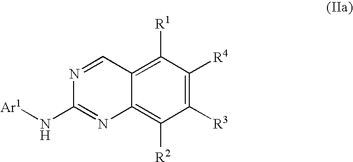
or pharmaceutically acceptable salt thereof, wherein:
R¹ is selected from H and halogen
R² is selected from H, halogen, and C₁₋₆ alkoxy;
R³ is selected from H, —OCH₂-phenyl, —O—CH₂-Het, —OCH₂—CH₂-Het, and —O—Hy; wherein Het is 6-membered heteroaryl which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; Hy is 6-membered heterocycloalkyl, which is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl; and phenyl is optionally substituted by 1 or 2 groups independently selected from hydroxyl, C₁₋₆ alkyl, C₁₋₄ alkoxy, amino, C₁₋₄ alkylamino, di-C₁₋₄ alkylamino, and C₁₋₆ alkylcarbamyl;
R⁴ is selected from cyano, halogen, C₁₋₆ alkyl, C₂₋₆ alkynyl, C₃₋₆ cycloalkyl, a thiazole ring, a pyrazole ring, and a pyridine ring; each of which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino; wherein said C₁₋₆ alkyl is further optionally substituted by 1 or 2 hydroxyl groups;
Ar¹ is a moiety of Group (A), (B), or (C): 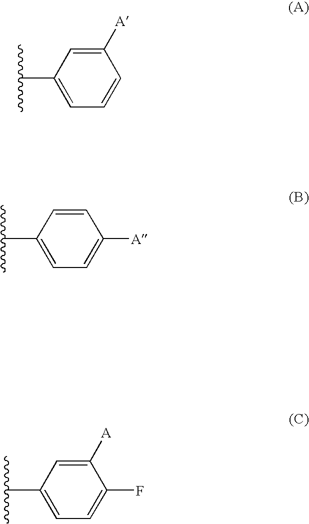
A is a pyrazole ring; which is optionally substituted by 1 or 2 groups independently selected from hydroxyl, C₁₋₆ alkyl, C₁₋₄ alkoxy, amino, C₁₋₄ alkylamino, and di-C₁₋₄ alkylamino;
A′ is selected from -L²-Ar² and -L¹-Cy¹;
A″ is selected from L^{2a}-Ar^{2a} and Cy^{1a};
L¹ is selected from a bond, —O—, —C(═O)—, —CH₂C(═O)—, and —CH₂—; wherein the left end of the linker is attached to the phenyl ring and the right end of the linker is attached to Cy¹;
Cy¹ is selected from a morpholine ring, a tetrahydro-2H-pyran ring, a pyrrolidine ring, a 2-oxopyrrolidine ring, and a piperidine ring, each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
Cy^{1a} is selected from a morpholine ring, a 2-oxopyrrolidine ring and a piperidine ring; each of which is optionally substituted by 1 or 2 groups independently selected from halogen, hydroxyl, C₁₋₆ alkyl, C₁₋₄ alkoxy, amino, C₁₋₄ alkylamino, di-C₁₋₄ alkylamino, C₁₋₆ alkylcarbonyl, and C₁₋₆ alkoxycarbonyl;
L² and L^{2a} are each independently selected from a bond, —O—, and —CH₂—; and
Ar² is selected from a pyrazole ring, an oxazole ring, an imidazole ring, a triazole ring, a thiadiazole ring, a pyridine ring, and a pyrimidine ring; each of which is optionally substituted with 1, 2, or 3 groups independently selected from hydroxyl, C₁₋₆ alkyl, C₁₋₄ alkoxy, amino, C₁₋₄ alkylamino, and di-C₁₋₄ alkylamino;
Ar^{2a} is selected from a pyrazole ring, a triazole ring, a pyridine ring, and a pyrimidine ring; each of which is optionally substituted with 1 or 2 groups independently selected from hydroxyl, C₁₋₆ alkyl, C₁₋₄ alkoxy, amino, C₁₋₄ alkylamino, and di-C₁₋₄ alkylamino;
provided that:
(i) when R¹ and R² are each H, R⁴ is thiazol-2-yl, R³ is optionally substituted piperidin-4-yloxy, and Ar¹ is a moiety of Group (A), then A′ is not selected from 1,2,4-triazol-1-ylmethyl, 1-methylpyrazol-3-yl, 2-oxopyrrolidinyl, oxazol-5-yl, pyrazol-1-yl, 1-methyl-1,2,4-triazol-2-yl, morpholin-4-ylcarbonylmethyl, imidazol-2-yl, 2-methylthiazol-4-yl, 1,3,5-trimethylpyrazol-4-yl, pyrimidin-5-yl, 1,2,4-triazol-1-yl, 4,5-dimethyloxazol-2-yl, pyrimidin-5-yl, 2-methoxypyrimidin-5-yl, 6-methoxypyridin-3-yl, and pyridin-3-yl;
(ii) when R¹ and R² are each H, Ar¹ is a moiety of Group (A), A′ is selected from 1-methylpyrazol-3-yl, and R³ is selected from pyridin-3-ylmethoxy and pyrazin-2-ylmethoxy, then R⁴ is not thiazol-2-yl;
(iii) when R¹ and R² are each H, R⁴ is thiazol-2-yl, R³ is optionally substituted piperidin-4-yloxy, and Ar¹ is a moiety of Group (B), then A″ is not selected from morpholin-4-yl, morpholin-4-ylcarbonylmethyl, pyrimidin-5-yl, and pyrazol-1-ylmethyl;
(iv) when R¹ and R² are each H, R⁴ is bromo, R³ is pyridin-3-ylmethyl, and Ar¹ is a moiety of Group (A), then A′ is not piperidin-4-yl;
(v) when R¹ and R³ are each H, R⁴ is bromo or ethynyl, R² is isopropoxy, and Ar¹ is a moiety of Group (A), then A′ is not selected from 1-methyl-1,2,4-triazol-2-ylmethyl and morpholin-4-ylcarbonylmethyl;
(vi) when R¹ and R² are each H, R⁴ is cyclopropyl, bromo or ethynyl, R³ is chloro, and Ar¹ is a moiety of Group (A), then A′ is not 1,2,4-triazol-1-ylmethyl;
(vii) when R¹ and R² are each H, R⁴ is ethynyl, R³ is chloro, and Ar¹ is a moiety of Group (A), then A′ is not imidazol-2-yl, pyrazol-1-yl, and morpholin-4-ylmethyl;
(viii) when R¹ and R³ are each H, R⁴ is ethynyl, R¹ is chloro, and Ar¹ is a moiety of Group (A), then A′ is not selected from pyrazol-1-yl, oxazol-5-yl, imidazol-2-yl, morpholin-4-ylcarbonylmethyl, and 2-oxopyrrolidinyl;
(ix) when R¹ and R² are each H, R⁴ is ethynyl, R³ is H, and Ar¹ is a moiety of Group (A), then A′ is not selected from 1,2,4-triazol-1-ylmethyl, pyrazol-1-yl, oxazol-5-yl, imidazol-2-yl, 5-trifluoromethylimidazol-2-yl, imidazol-1-ylmethyl, 1,2,4-triazol-1yl-methyl, N-(ethoxycarbonyl)piperidin-4-yl, N-(methoxycarbonyl)piperidin-4-yl, N-(acetyl)piperidin-4-yl, N-methylpiperidin-4-yl, piperidin-4-yl, and 2-oxopyrrolidinyl;
(x) when R¹ and R² are each H, R⁴ is ethynyl, Ar¹ is a moiety of Group (A), and A′ is 1-methylpyrazol-3-yl, then R³ is not selected from pyridin-3-ylmethoxy, 2-pyridin-3-ylethoxy, thiazol-5-ylmethoxy, and pyrazin-2-ylmethoxy;
(xi) when R¹ and R² are each H, R⁴ is ethynyl, Ar¹ is a moiety of Group (A), and A′ is morpholin-4-ylmethyl, then R³ is not selected from pyridin-3-ylmethoxy and pyrazin-2-ylmethoxy;
(xii) when R¹ and R² are each H, R⁴ is ethynyl, Ar¹ is a moiety of Group (A), and A′ is oxazol-5-yl, then R³ is not selected from pyridin-3-ylmethoxy and pyrazin-2-ylmethoxy;
(xiii) when R¹ and R² are each H, R⁴ is ethynyl, R³ is H, and Ar¹ is a moiety of Group (B), then A″ is not selected from morpholin-4-ylmethyl and 2-oxopyrrolidinyl;
(xiv) when R¹ and R² are each H, R⁴ is ethynyl, R³ is chloro, and Ar¹ is a moiety of Group (B), then A″ is not selected from 2-oxopyrrolidinyl and morpholin-4-ylmethyl;
(xv) when R¹ and R² are each H, R³ is pyrazin-2-ylmethoxy, R⁴ is pyrazol-4-yl, and Ar¹ is a moiety of Group (A), then A′ is not 1-methylpyrazol-3-yl;
(xvi) when R¹ and R² are each H, R³ is piperidin-4-yloxy or N-methylpiperidin-4-yloxy, R⁴ is pyrazol-4-yl, and Ar¹ is a moiety of Group (B), then A″ is not morpholin-4-yl;
(xvii) when Ar¹ is 3-morpholin-4-ylphenyl, then R³ is not H;
(xviii) when Ar¹ is 4-morpholin-4-ylphenyl, then R⁴ is not selected from bromo, cyano, ethynyl, thiazol-2-yl, and 1H-pyrazol-4-yl;
(xix) when R^{1′} is H, R^{2′} is H, R^{3′} is piperidin-4-yloxy, and R^{4′} is 6-methoxypyridin-3-yl, and Ar¹ is a moiety of Group (A), then A′ is not 6-methoxypyridin-3-yl; and
(xx) when R^{1′} is H, R^{2′} is H, R^{3′} is piperidin-4-yloxy, and R^{4′} is 5-methoxypyridin-3-yl, and Ar¹ is a moiety of Group (B), then A″ is not 5-methoxypyridin-3-yl.

11. The compound according to claim 14, or pharmaceutically acceptable salt thereof, wherein Ar¹ is a moiety of Group (A).

12. The compound according to claim 11, or pharmaceutically acceptable salt thereof, wherein A′ is -L²-Ar².

13. The compound according to claim 11, or pharmaceutically acceptable salt thereof, wherein A′ is -L¹-Cy¹.

14. The compound according to claim 13, or pharmaceutically acceptable salt thereof, wherein:
A′ is -L²-Ar²;
L² is a bond;
Ar² is selected from a pyridine ring, a pyrimidine ring, 1H-pyrazole ring, an oxazole ring, a 1,2,4-triazole ring, and a thiadiazole ring; each of which is optionally substituted by 1 or 2 groups independently selected from C₁₋₆ alkyl and C₁₋₆ alkoxy;
R¹ is H;
R² is H;
R³ is selected from —O-phenyl, —O—CH₂-Het, —OCH₂—CH₂-Het, and —O—Hy; wherein Het is a pyrazine ring, a pyridine ring, a pyrimidine ring, or a pyridazine ring; which is optionally substituted by a C₁₋₆ alkyl group; Hy is piperidine ring, which is optionally substituted by a C₁₋₆ alkyl group; and phenyl is optionally substituted by a C₁₋₆ alkylcarbamyl group; and
R⁴ is selected from C₂₋₆ alkynyl, thiazol-2-yl, 1H-pyrazol-4-yl, 1-methyl-1H-pyrazol-4-yl, and 1H-pyrazol-3-yl.

16. The compound according to claim 13, or pharmaceutically acceptable salt thereof, wherein:
A′ is -L²-Ar²;
L² is a bond;
Ar² is selected from 5-methoxypyridin-3-yl, pyrimidin-5-yl, 1H-pyrazol-3-yl, 1H-pyrazol-5-yl, oxazol-5-yl, 1,2,4-triazol-5-yl, thiadiazol-4-yl, and 1-methyl-1H-pyrazol-3-yl;
R¹ is H;
R² is H;
R³ is selected from piperidin-4-yloxy, N-methylpiperidin-4-yloxy, 3-(N-methylcarbamyl)benzyloxy, pyrazin-2-ylmethoxy, pyridin-3-ylmethoxy, pyridimidin-5-ylmethoxy, pyridazin-3-ylmethoxy, and pyrazin-2-ylethoxy; and
R⁴ is selected from ethynyl, thiazol-2-yl, 1H-pyrazol-4-yl, 1-methyl-1H-pyrazol-4-yl, and 1H-pyrazol-3-yl.

17. The compound according to claim 13, or pharmaceutically acceptable salt thereof, wherein:
A′ is -L²-Ar²;
L² is —CH₂—;
Ar² is selected from 1,2,4-triazol-1-yl, 1H-pyrazol-1-yl, and 1H-imidazol-1-yl ; each of which is optionally substituted by 1 or 2 independently selected C₁₋₆ alkyl groups;
R¹ is H;
R² is H;
R³ is selected from pyrazin-2-ylmethoxy, pyridin-3-ylmethoxy, pyrazin-2-ylethoxy, and pyrimidin-5-ylmethoxy; and
R³ is selected from —O—CH₂-Het and —OCH₂—CH₂-Het; wherein Het is a pyrazine ring, a pyridine ring, or a pyrimidine ring; which is optionally substituted by a C₁₋₆ alkyl group; and
R⁴ is selected from C₂₋₆ alkynyl, 1H-pyrazol-3-yl, 1H-pyrazol-4-yl, 1-methyl-1H-pyrazol-3-yl, 1-methyl-1H-pyrazol-4-yl, and 1-methyl-1H-pyrazol-5-yl.

18. The compound according to claim 13, or pharmaceutically acceptable salt thereof, wherein:
A′ is -L¹-Cy¹;
L¹ is selected from —CH₂— and —CH₂C(═O)—;
Cy¹ is selected from morpholin-4-yl; which is optionally substituted by 1 or 2 independently selected C₁₋₆ alkyl groups;
R¹ is H;
R² is H;
R³ is selected from —O—CH₂-Het and —OCH₂—CH₂-Het; wherein Het is a pyrazine ring, a pyridine ring, or a pyrimidine ring; which is optionally substituted by a C₁₋₆ alkyl group; and
R⁴ is selected from C₂₋₆ alkynyl, 1H-pyrazol-4-yl, 1-methyl-1H-pyrazol-4-yl, and 1H-pyrazol-3-yl.

19. The compound according to claim 13, or pharmaceutically acceptable salt thereof, wherein:
A′ is -L¹-Cy¹;
L¹ is selected from —CH₂— and —CH₂C(═O)—;
Cy¹ is morpholin-4-yl;
R¹ is H;
R² is H;
R³ is selected from pyrazin-2-ylmethoxy, pyridin-3-ylmethoxy, pyrimidin-5-ylmethoxy, and pyrazin-2-ylethoxy; and
R⁴ is selected from ethynyl, 1H-pyrazol-4-yl, 1-methyl-1H-pyrazol-4-yl, and 1H-pyrazol-3-yl.

20. The compound according to claim 13, or pharmaceutically acceptable salt thereof, wherein:
A′ is -L¹-Cy¹;
L¹ is —C(═O)—;
Cy¹ is selected from a morpholine ring; which is optionally substituted by 1 or 2 independently selected C₁₋₆ alkyl groups;
R¹ is H;
R² is H;
R³ is selected from —O—CH₂-Het and —OCH₂—CH₂-Het; wherein Het is a pyrazine ring; which is optionally substituted by a C₁₋₆ alkyl group; and
R⁴ is selected from cyano, methyl, C₂₋₆ alkynyl, C₃₋₆ cyclopropyl, 1-methyl-1H-pyrazol-4-yl, 1H-pyrazol-4-yl, and 1H-pyrazol-3-yl.

21. Use of a compound according to any one of claims 1 to 20, for the preparation of medicament for use in a method of treating a cancer selected from lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, pharyngeal cancer, laryngeal cancer, small intestinal cancer, non-Hodgkin lymphoma, and villous colon adenoma.

22. Use of a compound according to any one of claims 1 to 20, for the preparation of medicament for use in a method of inhibiting PDK1 or a PDK1 variant in an individual.

23. A compound according to any one of claims 1 to 20, for use in a method Of treating a cancer selected from lung cancer, bronchial cancer, prostate cancer, breast cancer, pancreatic cancer, colon cancer, rectal cancer, colorectal cancer, thyroid cancer, liver cancer, intrahepatic bile duct cancer, hepatocellular cancer, gastric cancer, glioma/glioblastoma, endometrial cancer, melanoma, kidney cancer, renal pelvic cancer, urinary bladder cancer; uterine corpus cancer; uterine cervical cancer, ovarian cancer, multiple myeloma, esophageal cancer, acute myelogenous leukemia, chronic myelogenous leukemia, lymphocytic leukemia, myeloid leukemia, brain cancer, oral cavity cancer, pharyngeal cancer, laryngeal cancer, small intestinal cancer, non-Hodgkin lymphoma, and villous colon adenoma.

24. A compound according to any one of claims 1 to 20, for use in a method of a treating a disease selected from neuro-fibromatosis, atherosclerosis, pulmonary fibrosis, arthritis, psoriasis, glomerulonephritis, restenosis, proliferative diabetic retinopathy, hypertrophic scar formation, inflammatory bowel disease, transplantation rejection, angiogenesis and endotoxic shock.

25. A compound according to any one of claims 1 to 20, for use in a method of inhibiting the tumor growth in an individual.

26. A compound according to any one of claims 1 to 20, for use in a method of inhibiting PDK1 or a PDK1 variant in an individual.
